(19) United States
(12) Patent Application Publication
(10) Pub. No.: US 2026/0217687 A1
(43) Pub. Date: Jul. 30, 2026

(54) MLH1 SELECTIVE (SERIES 1B) COMPOUNDS

(71) Applicant: NeoPhore Limited, Altrincham (GB)

(72) Inventors: Julian Blagg, Altrincham (GB); Martin Drysdale, Altrincham (GB); Paul Winship, Saffron Walden (GB); Stuart Rankin, Saffron Walden (GB); Alastair Rae, Saffron Walden (GB); Jacob Bestwick, Saffron Walden (GB)

(21) Appl. No.: 19/148,966

(22) PCT Filed: Jan. 19, 2024
(86) PCT No.: PCT/GB2024/050147

§ 371 Date: Jul. 17, 2025

(30) Foreign Application Priority Data
Jan. 20, 2023 (GB) 2300881.6

(51) Int. Cl. C07D 403/12; A61K 31/4035; A61K 31/404; A61K 31/4178; A61K 31/422; A61K 31/437; A61K 31/4439; A61K 31/454; A61K 31/4545; A61K 31/4709; A61K 31/4725; A61K 31/496; A61K 31/506; A61K 31/519; A61K 31/5377; A61K 31/55; A61K 45/06; C07D 209/08; C07D 209/44; C07D 217/06; C07D 401/06; C07D 401/12; C07D 401/14; C07D 403/06; C07D 405/12; C07D 405/14; C07D 413/12; C07D 471/04

(52) U.S. Cl. CPC C07D 403/12; A61K 31/4035; A61K 31/404; A61K 31/4178; A61K 31/422; A61K 31/437; A61K 31/4439; A61K 31/454; A61K 31/4545; A61K 31/4709; A61K 31/4725; A61K 31/496; A61K 31/506; A61K 31/519; A61K 31/5377; A61K 31/55; A61K 45/06; C07D 209/08; C07D 209/44; C07D 217/06; C07D 401/06; C07D 401/12; C07D 401/14; C07D 403/06; C07D 405/12; C07D 405/14; C07D 413/12; C07D 471/04

(57) ABSTRACT

The present invention relates to compounds of Formula (I) that target MLH1, a component of the DNA Mismatch Repair (MMR) process:

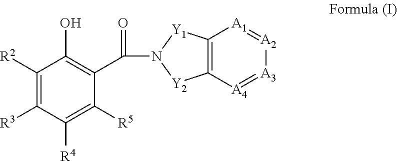

wherein R², R³, R⁴, R⁵, Y₁, Y₂, A₁, A₂, A₃, and A₄ are each as defined herein. The present invention also relates to processes for the preparation of these compounds, to pharmaceutical compositions comprising them, and to their use in the treatment of proliferative disorders, such as cancer, as well as other diseases or conditions in which MLH1 activity is implicated (e.g. Triplet Repeat Disorders).

### INTRODUCTION

The present invention relates to certain compounds that function as selective inhibitors of MLH1. The compounds of the present invention may be used to treat disease or conditions mediated, at least in part, by inappropriate MLH1 activity, for example cancer. The invention furthermore relates to the use of the compounds as pharmaceuticals, processes for making them and pharmaceutical compositions comprising them.

### BACKGROUND OF THE INVENTION

Cancer is caused by altered cellular proliferation. Precisely what causes a cell to become malignant and proliferate in an uncontrolled and unregulated manner has been the focus of intense research over recent decades. This research has led to the identification of molecular targets associated with key pathways that enable such malignancies.

Mismatch repair (MMR) is a highly conserved DNA repair pathway that plays a major role during DNA replication, repair, and recombination, as well as during meiosis in eukaryotes and immunoglobulin maturation/diversification in mammals. MMR promotes genome stability in all organisms by correcting DNA base mismatches and insertion/deletion (indel) loops that can occasionally arise during normal DNA replication process. Base pair mismatches occur when incorrect nucleotides are inserted into the newly synthesized DNA strand and escape the proofreading function of DNA polymerases. Indel loops commonly arise in the context of microsatellites—highly polymorphic short repetitive DNA sequences distributed throughout both prokaryotic and eukaryotic genomes. Typically, at microsatellites, the template and primer strands are prone to slippage (dissociation and reannealing) during replication, which can generate loop structures and a discordant number of repeat units between the template and newly synthesized strand.

DNA mismatch repair is a bidirectional excision and re-synthesis system that initiates at a defined strand scission 3′- or 5′- to a mismatch; the excision tract extends just past the mismatch. MMR can be divided into four steps: 1) mismatch recognition by MSH proteins; 2) recruitment of MLH proteins that connect the mismatch recognition signal to where the distant DNA strand scission begins; 3) excision of the errant DNA strand, and 4) re-synthesis of the excision gap using the remaining DNA strand as a template [1]. MMR is a highly conserved biological pathway. In humans, mismatch recognition by hMutSα (MSH2-MSH6) or hMutSβ (MSH2-MSH3) initiates the MMR pathway. Binding of hMutSα or hMutSβ to the mismatch site results in the recruitment of MutLα (MLH1-PMS2) to form a ternary complex whose protein-protein and protein-DNA interactions are modulated by ATP/ADP cofactors. Proliferating cell nuclear antigen (PCNA) may play a role in the recruitment of MMR proteins to the vicinity of the replication fork [1]. PCNA may also activate a latent endonuclease activity in eukaryotic MutLα proteins. After DNA incision, exonuclease 1 (EXO1) is recruited which excises the newly synthesized DNA strand and the DNA excision gap is re-synthesized by DNA polymerase 6 (Pol δ). When DNA re-synthesis is complete, the remaining nick is ligated by DNA ligase to restore the integrity of the duplex [2]. Consistent with this function, MMR is an important tumor suppressor pathway that is lost in up to 40% of sporadic cancers. Moreover, individuals with germline mutations in MMR genes develop cancer predisposition conditions.

Lynch Syndrome (LS, formerly designated as hereditary non-polyposis colorectal cancer) is the most common cause of hereditary colorectal cancer (CRC), accounting for 2-5% of all cases. LS is also characterized by an increased risk of malignancies at certain extracolonic sites such as the endometrium, ovary, stomach and small bowel, among others [3]. LS has an autosomal dominant inheritance pattern and is caused by germline mutations in MMR genes MLH1, MSH2, MSH6 or PMS2. Gene expression from the one wild-type allele is sufficient for adequate MMR activity until a second hit inactivates the wild-type allele leading to MMR deficiency.

Constitutional mismatch repair deficiency (CMMRD) syndrome is a distinct childhood cancer predisposition syndrome that results from biallelic germline mutations in one of the four MMR genes, MLH1, MSH2, MSH6 or PMS2. Patients may have either homozygous biallelic alterations or heterozygous alterations of MMR genes.

MMR-deficient cancers are commonly and typically characterized by the accumulation of DNA mutations at higher rates than normal cells and other tumours; for example, CMMRD tumours commonly have an ultra-hypermutated phenotype (>250 substitution mutations/Mb) [4]. MMR deficiency also results in gains or losses in the repeat length of microsatellites, referred to as microsatellite instability (MSI). Cancers that possess more than 40% microsatellite variations (positive for two or more of five microsatellite markers routinely tested) are described as high frequency MSI (MSI-H). Tumours that have no MSI are microsatellite stable (MSS) and those that possess less than 40% microsatellite variations (one out of the five markers showing microsatellite instability) are low frequency MSI (MSI-L) [5]. MSI analysis is a widely used diagnostic biomarker of MMR-deficient tumours and MSI status is linked with a high prevalence of frameshift (FS) mutations that can occur because of insertion/deletion within coding microsatellites. In addition to altering downstream functions of the protein, the FS creates a new amino acid sequence that serves as a substrate for antigen processing and presentation [6], stimulating the activation of CD8+ T cells (class I) and the “helper” function of CD4+ T cells (class II).

Cancers with a greater number of neoantigens are more prone to immune surveillance and have an increased likelihood of responding to immunotherapy [7]; higher neoantigen load is associated with overall lymphocytic infiltration, TILs, memory T cells, and survival in colorectal cancer [8, 9]. This feature supports a rationale for immunotherapy-based treatment strategies [6]. Consistent with this notion, immune checkpoint inhibitors now offer a significant therapeutic advance in the treatment of MMR-deficient (MMR-D) cancers. Inhibitors of PD-1; for example, pembrolizumab (Keytruda) and nivolumab (Opdivo), have been approved by the Food and Drug Administration (FDA) for patients with MMR-D or MSI-H metastatic CRC based upon the significant survival benefit they provide. The CTLA-4 inhibitor ipilimumab (Yervoy), has been approved for use in combination with nivolumab for the treatment of MMR-D or MSI-H CRC patients who were previously treated with chemotherapy. Importantly, the FDA has approved the use of pembrolizumab in MMR-D/MSI-H cancers regardless of histological tumour type [10].

It is now accepted that clinical responses to immune checkpoint inhibitors require the existence of tumour neoantigens and infiltration of T cells that recognize such neoantigens. Higher neoantigen load is associated with response to CTLA-4 and PD-1 blockade in patients with melanoma and non-small-cell lung cancer [11, 12, 13]. The number of neoantigens is linked to TMB, and several large studies have confirmed that high TMB correlates with enhanced checkpoint inhibitor responses and improved overall survival in certain tumour types, such as urothelial carcinoma [14], non-small cell lung cancer [15-18] and small cell lung cancer [19].

Germano et al. recently proposed that MMR inactivation through silencing of MLH1 increases TMB and leads to “dynamic mutational profiles”, resulting in persistent renewal of neoantigens both in vitro and in vivo. This triggers immune surveillance and leads to the control of tumour growth, particularly in combination with immune checkpoint inhibition, in mouse models [20]. Similar results are observed upon silencing of MSH2 [21].

Guan et al. and Lu et al. report that MLH1 deficiency leads to cytosolic DNA release, activation of the cGAS-STING pathway and IFN-3 production. Guan et al. demonstrate that MLH1 loss leads to DNA hyperexcision, RPA exhaustion, chromosomal instability and accumulation of cytosolic DNA [22]. Lu et al. report that the sensing of cytosolic DNA by the cGAS STING pathway contributes to the clinical benefit of immunotherapy in patients harboring MMR deficient tumours [23]. Together these reports suggest that abrogation of MMR activity may elicit beneficial immune activation through activation of the cGAS-STING pathway.

There is therefore a robust biological and clinical rationale highlighting the need for inhibitors that target the MLH1 proteins, key components of DNA MMR to reawaken an anti-tumour immune response.

Thus, the present invention provides methods for the treatment of cancer by binding to and modulating the function of the DNA MMR component MLH1 using small molecules as single agents and in combination with immunotherapy agents, other DNA damage response pathway modulators and/or standard-of-care chemotherapeutic agents.

Outside of the cancer field, triplet repeat disorders comprise over 30 human neurodegenerative and neuromuscular inherited diseases such as Huntington's disease (HD), myotonic dystrophy type 1 (DM1), fragile X syndrome type A (FRAXA), Friedreich's ataxia (FRDA), and spinocerebellar ataxias (SCAs). Such disorders are characterized by the expansion of simple repeats in genomic DNA. These unstable repeats are commonly found at different regions of several genes and their expansion can cause disease by a variety of both loss- and gain-of-function pathways, for instance through interfering with the expression or properties of the gene products, or by affecting splicing or antisense regulation. Several mechanisms including errors during DNA replication, meiotic recombination, transcription, DNA repair, and chromatin remodeling have been proposed to contribute to repeat instability, which can occur at various stages of the cell cycle. There is evidence that a functional MMR pathway is required for maintaining the stability of microsatellite sequences: for example, Msh2−/− transgenic mice bearing a copy of the human HD exon 1 (containing the CAG repeats) showed reduced expansion of the introduced (CAG)n repeats when compared with Msh2+/+HD exon 1 mice counterparts [24].

Thus, there is a further need for compounds that selectively target MLH1 components of the DNA MMR process for treating triplet repeat disorders. The present invention was devised with the foregoing in mind.

### REFERENCES

1. Martin-Lopez, J. V. and R. Fishel, The mechanism of mismatch repair and the functional analysis of mismatch repair defects in Lynch syndrome. Fam Cancer, 2013. 12(2): p 159-68.
2. Liu, D., G. Keijzers, and L. J. Rasmussen, DNA mismatch repair and its many roles in eukaryotic cells. Mutat Res, 2017. 773: p. 174-187.
3. Lynch, H. T., et al., Review of the Lynch syndrome: history, molecular genetics, screening, differential diagnosis, and medicolegal ramifications. Clin Genet, 2009. 76(1): p. 1-18.
4. Shlien, A., et al., Combined hereditary and somatic mutations of replication error repair genes result in rapid onset of ultra-hypermutated cancers. Nat Genet, 2015. 47(3): p 257-62
5. Sehgal, R., et al., Lynch syndrome: an updated review. Genes (Basel), 2014. 5(3): p. 497-507
6. Willis, J. A., et al., Immune Activation in Mismatch Repair-Deficient Carcinogenesis: More Than Just Mutational Rate. Clin Cancer Res, 2019.
7. Gubin, M. M. and R. D. Schreiber, CANCER. The odds of immunotherapy success. Science, 2015. 350(6257): p. 158-9.
8. Kloor, M. and M. von Knebel Doeberitz, The Immune Biology of Microsatellite-Unstable Cancer. Trends Cancer, 2016. 2(3): p. 121-133.
9. Giannakis, M., et al., Genomic Correlates of Immune-Cell Infiltrates in Colorectal Carcinoma. Cell Rep, 2016. 17(4): p. 1206.
10. Lemery, S., P. Keegan, and R. Pazdur, First FDA Approval Agnostic of Cancer Site—When a Biomarker Defines the Indication. N Engl J Med, 2017. 377(15): p. 1409-1412.
11. Le, D. T., et al., PD-1 Blockade in Tumors with Mismatch-Repair Deficiency. N Eng J Med, 2015. 372(26): p. 2509-20.
12. Rizvi, N. A., et al., Cancer immunology. Mutational landscape determines sensitivity to PD—1 blockade in non-small cell lung cancer. Science, 2015. 348(6230): p. 124-8.
13. Van Allen, E. M., et al., Genomic correlates of response to CTLA-4 blockade in metastatic melanoma. Science, 2015. 350(6257): p. 207-211.
14. Rosenberg, J E., et al., Atezolizumab in patients with locally advanced and metastatic urothelial carcinoma who have progressed following treatment with platinum-based chemotherapy: a single-arm, multicentre, phase 2 trial. Lancet, 2016. 387(10031): p. 1909-20.
15. Hellmann, M. D., et al., Genomic Features of Response to Combination Immunotherapy in Patients with Advanced Non-Small-Cell Lung Cancer. Cancer Cell, 2018. 33(5): p. 843-852 e4.
16. Rizvi, H., et al., Molecular Determinants of Response to Anti-Programmed Cell Death (PD)-1 and Anti-Programmed Death-Ligand 1 (PD-L1) Blockade in Patients With Non-Small-Cell Lung Cancer Profiled With Targeted Next-Generation Sequencing. J Clin Oncol, 2018. 36(7): p. 633-641.
17. Carbone, D. P., et al., First-Line Nivolumab in Stage IV or Recurrent Non-Small-Cell Lung Cancer. N Engl J Med, 2017. 376(25): p. 2415-2426.
18. Hellmann, M. D., et al., Nivolumab plus Ipilimumab in Lung Cancer with a High Tumor Mutational Burden. N Engl J Med, 2018. 378(22): p. 2093-2104.
19. Hellmann, M. D., et al., Tumor Mutational Burden and Efficacy of Nivolumab Monotherapy and in Combination with Ipilimumab in Small-Cell Lung Cancer. Cancer Cell, 2018. 33(5): p. 853-861 e4.
20. Germano, G., et al., Inactivation of DNA repair triggers neoantigen generation and impairs tumour growth. Nature, 2017. 552(7683): p. 116-120.
21. Mandal, R., et al., Genetic diversity of tumors with mismatch repair deficiency influences anti-PD-1 immunotherapy response. Science, 2019. 364(6439): p. 485-491.
22. Guan J., et al., MLH1 deficiency-triggered DNA hyperexcision by exonuclease 1 activates the cGAS-STING pathway. Cancer Cell. 2021, 39 (1), 109-121,
23. Lu, C., et al. DNA sensing in mismatch repair-deficient tumor cells is essential for anti—tumor immunity. Cancer Cell. 2021, 39 (1), 96-108.
24. Manley, K., et al., Msh2 deficiency prevents in vivo somatic instability of the CAG repeat in Huntington disease transgenic mice. Nat Genet, 1999. 23(4): p. 471-3.

### SUMMARY OF THE INVENTION

According to a first aspect of the present invention there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of inhibiting MLH1 activity, in vitro or in vivo, said method comprising contacting a cell with an effective amount of a compound or a pharmaceutically acceptable salt, hydrate or solvate thereof as defined herein.

According to a further aspect of the present invention, there is provided a method of treating a disease or disorder in which MLH1 activity is implicated in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a triplet disorder (e.g. Huntington's disease (HD), myotonic dystrophy type 1 (DM1), fragile X syndrome type A (FRAXA), Friedreich's ataxia (FRDA), and spinocerebellar ataxias (SCAs)) in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein for use in therapy.

According to a further aspect of the present invention, there is provided a compound, or a pharmaceutically acceptable salt, hydrate or solvate thereof, or a pharmaceutical composition as defined herein for use as a medicament.

According to a further aspect of the present invention, there is provided a compound or a pharmaceutically acceptable salt, hydrate or solvate thereof as defined herein, or a pharmaceutical composition as defined herein, for use in the treatment of a proliferative disorder.

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer.

According to a further aspect of the present invention, there is provided a compound, or a pharmaceutically acceptable salt, hydrate or solvate thereof, or a pharmaceutical composition as defined herein for use in the treatment of a triplet disorder. In a particular embodiment, the triplet disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided a compound, or a pharmaceutically acceptable salt, hydrate or solvate thereof, as defined herein for use in the inhibition of MLH1 activity.

According to a further aspect of the present invention, there is provided a compound, or a pharmaceutically acceptable salt, hydrate or solvate thereof, as defined herein for use in the treatment of a disease or disorder in which MLH1 activity is implicated.

According to a further aspect of the present invention, there is provided the use of a compound, or a pharmaceutically acceptable salt, hydrate or solvate thereof, as defined herein in the manufacture of a medicament for the treatment of a proliferative disorder.

According to a further aspect of the present invention, there is provided the use of a compound, or a pharmaceutically acceptable salt, hydrate or solvate thereof, as defined herein in the manufacture of a medicament for the treatment of cancer.

According to a further aspect of the present invention, there is provided the use of a compound, or a pharmaceutically acceptable salt, hydrate or solvate thereof, as defined herein in the manufacture of a medicament for the treatment of a triplet disorder. In a particular embodiment, the triplet disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided a use of a compound, or a pharmaceutically acceptable salt, hydrate or solvate thereof, as defined herein in the manufacture of a medicament for the inhibition of MLH1 activity.

According to a further aspect of the present invention, there is provided a use of a compound, or a pharmaceutically acceptable salt, hydrate or solvate thereof, as defined herein in the manufacture of a medicament for the treatment of a disease or disorder in which MLH1 activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

In the above-outlined aspects of the invention, the proliferative disorder is suitably cancer, and the cancer is suitably a human cancer. In particular, the compounds of the present invention will be useful for the treatment of any cancer in which mis-match repair inhibition and/or cGAS/STING pathway activation is beneficial. Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dube Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

### Probe Compounds

The present invention also resides in the discovery of a new series of probe compounds that effectively bind to the ATP-binding site of MLH1. These probe compounds can be used in assays and methods for assessing the binding affinity of test compounds to the ATP-binding site of MLH1. In addition, these probe compounds can be used to determine the location and/or quantity of MLH1 in a biological sample.

Thus, in another aspect, the present invention provides a probe compound of formula (I), or a salt thereof, as defined herein, wherein one of R¹² or R¹³ is a group L-Q or Lₓ-X as defined herein.

In another aspect, the present invention provides a method of synthesising a probe compound of formula (I), or a salt thereof, as defined herein.

In another aspect, the present invention provides the use of a probe compound of formula (I), or a salt thereof, in a displacement assay to determine the binding affinity of a test molecule to the ATP-binding site of MLH1.

In another aspect, the present invention provides a probe compound of formula (I), or a salt thereof, for use in a displacement assay to determine the binding affinity of a test molecule for the ATP-binding site of MLH1.

In another aspect, the present invention provides an assay for determining the binding affinity of a test molecule for the ATP-binding site of MLH1, the assay comprising:
(i) incubating a test molecule with the target protein in the presence of a probe compound of formula (I), or a salt thereof, as defined herein; and
(ii) determining whether a probe compound of the formula (I) is displaced from the ATP-binding site of the target protein.

In another aspect, the present invention provides a method for determining the binding affinity of a test molecule for the ATP-binding site of MLH1, the assay comprising:
(i) incubating a test molecule with the target protein in the presence of a probe compound of formula (I), or a salt thereof, as defined herein; and
(ii) determining whether a probe compound of the formula (I) is displaced from the ATP-binding site of the target protein.

In another aspect, the present invention provides an assay for determining the location and/or quantity of MLH1 present within a biological sample, the assay comprising:
(i) contacting the biological sample with a probe compound of formula (I), or a salt thereof, as defined herein; and
(ii) determining location and/or quantity of the compound of formula (I) present within the sample by detecting the location and/or intensity of the detection moieties of the compounds of formula (I) present within the biological sample.

In another aspect, the present invention provides an assay for determining the location and/or quantity of MLH1 present within a biological sample, the assay comprising:
(i) contacting the biological sample with a probe compound of formula (I), or a salt thereof, wherein one of R¹² or R¹³ is a group Lₓ-X as defined herein; and
(ii) contacting the biological sample with a detection moiety Q, or a compound of the formula Q-L₂-Y as defined herein, that is capable of reacting with the functional group X present on the compound of formula (I) to form a compound of formula (I) wherein one of R¹² or R¹³ is a group L-Q as defined herein in situ within the biological sample;
(iii) determining location and/or quantity of the compound of formula (I) present within the sample by detecting the location and/or intensity of the detection moieties Q of the probe compounds of formula (I) present within the biological sample.

In another aspect, the present invention provides a method for determining the location and/or quantity of MLH1 present within a biological sample, the method comprising:
(i) contacting the biological sample with a probe compound of formula (I), or a salt thereof, as defined herein; and
(ii) determining location and/or quantity of the compound of formula (I) present within the sample by detecting the location and/or intensity of the detection moieties Q of the probe compounds of formula (I) present within the biological sample.

In another aspect, the present invention provides a method for determining the location and/or quantity of MLH1 present within a biological sample, the method comprising:
(i) contacting the biological sample with a probe compound of formula (I), or a salt thereof, wherein one of R¹² or R¹³ is a group Lₓ-X as defined herein; and
(ii) contacting the biological sample with a detection moiety Q, or a compound of the formula Q-L₂-Y as defined herein, that is capable of reacting with the functional group X present on the compound of formula (I) to form a probe compound of formula (I) wherein one of R¹² or R¹³ is a group L-Q as defined herein in situ within the biological sample;
(iii) determining location and/or quantity of the compound of formula (I) present within the sample by detecting the location and/or intensity of the detection moieties Q of the compounds of formula (I) present within the biological sample.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. It should be understood that in, for example, a human or other mammal, a therapeutically effective amount can be determined experimentally in a laboratory or clinical setting, or a therapeutically effective amount may be the amount required by the guidelines of the United States Food and Drug Administration (FDA) or equivalent foreign regulatory body, for the particular disease and subject being treated. It should be appreciated that determination of proper dosage forms, dosage amounts, and routes of administration is within the level of ordinary skill in the pharmaceutical and medical arts.

As used herein by themselves or in conjunction with another term or terms, “subject(s)” and “patient(s)”, refer to animals (e.g. mammals), particularly humans. Suitably, the “subject(s)” and “patient(s)” may be a non-human animal (e.g. livestock and domestic pets) or a human.

As used herein by itself or in conjunction with another term or terms, “pharmaceutically acceptable” refers to materials that are generally chemically and/or physically compatible with other ingredients (such as, for example, with reference to a formulation), and/or is generally physiologically compatible with the recipient (such as, for example, a subject) thereof.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl.

The term “(m-nC)” or “(m-nC) group” used alone or as a prefix, refers to any group having m to n carbon atoms.

An “alkylene” group is an alkyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene (—CH₂—), the ethylene isomers (—CH(CH₃)— and —CH₂CH₂—), the propylene isomers (—CH(CH₃)CH₂—, —CH(CH₂CH₃)—, —C(CH₃)₂—, and —CH₂CH₂CH₂—), pentylene (—CH₂CH₂CH₂CH₂CH₂—), and the like.

The term “alkenyl” refers to straight and branched chain alkyl groups comprising 2 or more carbon atoms, wherein at least one carbon-carbon double bond is present within the group. Examples of alkenyl groups include ethenyl, propenyl and but-2,3-enyl and includes all possible geometric (E/Z) isomers.

The term “alkynyl” refers to straight and branched chain alkyl groups comprising 2 or more carbon atoms, wherein at least one carbon-carbon triple bond is present within the group. Examples of alkynyl groups include acetylenyl and propynyl.

“(m-nC)cycloalkyl” means a saturated hydrocarbon ring system containing from m to n number of carbon atoms. Exemplary cycloalkyl groups include, for example, cyclopropyl, cyclobutyl, cyclopentyl, cyclohexyl, cycloheptyl and bicyclo[2.2.1]heptyl.

The term “alkoxy” refers to O-linked straight and branched chain alkyl groups. Examples of alkoxy groups include methoxy, ethoxy and t-butoxy.

The term “haloalkyl” is used herein to refer to an alkyl group in which one or more hydrogen atoms have been replaced by halogen (e.g. fluorine) atoms. Examples of haloalkyl groups include —CH₂F, —CHF₂ and —CF₃.

The term “halo” or “halogeno” refers to fluoro, chloro, bromo and iodo, suitably fluoro, chloro and bromo, more suitably, fluoro and chloro.

The term “carbocyclyl”, “carbocyclic” or “carbocycle” means a non-aromatic saturated or partially saturated monocyclic, fused, bridged, or spiro bicyclic carbon-containing ring system(s). Monocyclic carbocyclic rings contain from about 3 to 12 (suitably from 3 to 7) ring atoms. Bicyclic carbocycles contain from 6 to 17 member atoms, suitably 7 to 12 member atoms, in the ring. Bicyclic carbocyclic(s) rings may be fused, spiro, or bridged ring systems. Examples of carbocyclic groups include cyclopropyl, cyclobutyl, cyclohexyl, cyclohexenyl and spiro[3.3]heptanyl.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydro-oxathiolyl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. Heterocycles may comprise 1 or 2 oxo (═O) or thioxo (═S) substituents. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

As used herein by itself or in conjunction with another term or terms, “aromatic” refers to monocyclic and polycyclic ring systems containing 4n+2 pi electrons, where n is an integer. Aromatic should be understood as referring to and including ring systems that contain only carbon atoms (i.e. “aryl”) as well as ring systems that contain at least one heteroatom selected from N, O or S (i.e. “heteroaromatic” or “heteroaryl”). An aromatic ring system can be substituted or unsubstituted.

As used herein by itself or in conjunction with another term or terms, “non-aromatic” refers to a monocyclic or polycyclic ring system having at least one double bond that is not part of an extended conjugated pi system. As used herein, non-aromatic refers to and includes ring systems that contain only carbon atoms as well as ring systems that contain at least one heteroatom selected from N, O or S. A non-aromatic ring system can be substituted or unsubstituted.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In a particular embodiment, an aryl is phenyl.

This specification also makes use of several composite terms to describe groups comprising more than one functionality. Such terms will be understood by a person skilled in the art. For example (3-6C)cycloalkyl(m-nC)alkyl comprises (m-nC)alkyl substituted by (3-6C)cycloalkyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups. In some embodiments, one or more refers to one, two or three. In another embodiment, one or more refers to one or two. In a particular embodiment, one or more refers to one.

The phrase “compound of the invention” means those compounds which are disclosed herein, both generically and specifically.

“About” when used herein in conjunction with a measurable value such as, for example, an amount or a period of time and the like, is meant to encompass reasonable variations of the value, for instance, to allow for experimental error in the measurement of said value.

### Compounds

In one aspect, the present invention relates to a compound, or a pharmaceutically acceptable salt thereof, having the structural Formula (I) shown below:

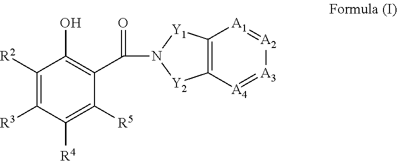

wherein
R² is hydrogen or fluoro;
R³ is selected from halo, cyano, (1-2C)alkyl, (1-2C)haloalkyl, (1-2C)hydroxyalkyl, (1-2C)alkoxy, (1-2C)haloalkoxy;
R⁴ is selected from hydrogen, halo or cyano;
or R³ and R⁴ are linked to form:
   a fused 5- or 6-membered carbocyclic or heterocyclic ring;
   a fused phenyl ring; or
   a 5- or 6-membered heteroaryl ring;
   each of which is optionally further substituted by halo, hydroxy, cyano, (1-2C)alkyl, (1-2C)haloalkyl, (1-2C)alkoxy or (1-2C)haloalkoxy;
R⁵ is R⁶ or —OR⁶;
   wherein R⁶ is (1-6C)alkyl, (3-8C)cycloalkyl, a 4- to 7-membered heterocyclyl ring comprising one heteroatom selected from N, O or S, phenyl, or a 5- or 6-membered heteroaryl; each of which is optionally substituted by one or more substituents independently selected from the group consisting of hydroxy, cyano, halogen, (1-3C)alkyl, (1-3C)alkoxy, (1-3C)haloalkyl, or (1-3C)haloalkoxy;
   or a group having a structure according to formula (A) shown below:

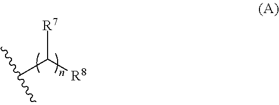

wherein
   R⁷ is hydrogen or (1-3C)alkyl;
   n is 1 or 2;
   R⁸ is (3-8C)cycloalkyl, aryl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
Y₁ is a bond, —CH₂—, —CH₂—CH₂— or —C(═O)—;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl, provided that when Y₁ is a bond, Y₂ is —CH₂—CH₂—, —CH₂—CHR_{y2b}—, or —CHR_{y2a}—CH₂—;
A₁ is selected from N, CH or CR¹¹;
A₂ is selected from N, CH or CR¹²;
A₃ is selected from N, CH or CR¹³;
A₄ is selected from N, CH or CR¹⁴
   with the proviso that only one or two of A₁, A₂, A₃ or A₄ can be N;
R¹¹ is cyano, halo, (1-2C)alkyl, (1-2C)alkoxy, wherein any (1-2C)alkyl or (1-2C)alkoxy is optionally substituted by one or more halo or (1-2C)alkoxy;
   or R¹¹ is —(CHR_{z})ₐ—Z¹¹,
      wherein:
      R_{z} is hydrogen or methyl;
      a is 0 or 1; and
      Z¹¹ is —OR¹⁵, R^{15a}, —NR¹⁶R¹⁷, —C(O)NR¹⁶R¹⁷ or —NR¹⁸C(O)R¹⁹;
         wherein
         R¹⁵ is (1-4C)alkyl, (3-7C)cycloalkyl, phenyl, a carbon-linked 4 to 8-membered heterocyclyl, a 5 or 6 membered heteroaryl, —(CHR_{y})_{b}-(3-7C)cycloalkyl, —(CHR_{y})_{b}-phenyl, —(CHR_{y})_{b}-[4 to 8-membered heterocyclyl] or —(CHR_{y})_{b}-[5 or 6 membered heteroaryl], wherein R_{y} is hydrogen or methyl and b is 0, 1 or 2;
         R^{15a} is (1-4C)alkyl, (3-7C)cycloalkyl, phenyl, 4 to 8-membered heterocyclyl, a 5 or 6 membered heteroaryl, —(CHR_{y})_{b}-(3-7C)cycloalkyl, —(CHR_{y})_{b}-phenyl, —(CHR_{y})_{b}-[4 to 8-membered heterocyclyl] or —(CHR_{y})_{b}-[5 or 6 membered heteroaryl], wherein R_{y} is hydrogen or methyl and b is 0, 1 or 2;
         R¹⁶ and R¹⁷ are each independently selected from hydrogen, (1-6C)alkyl, (3-7C)cycloalkyl, phenyl, a carbon-linked 4 to 8-membered heterocyclyl, a 5 or 6 membered heteroaryl, —(CHR_{w})_{c}-(3-7C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{w} is hydrogen or methyl and c is 0, 1 or 2;
         or R¹⁶ and R¹⁷ are linked, such that, together with the nitrogen atom to which they are attached, they form a 4-8 membered heterocyclic ring;
         R¹⁸ is hydrogen or (1-2C)alkyl;
         R¹⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₓ)_{d}-(3-7C)cycloalkyl, —(CHRₓ)_{d}-phenyl, —(CHRₓ)_{d}-[4 to 8-membered heterocyclyl] or —(CHRₓ)_{d}-[5 or 6 membered heteroaryl];
         wherein Rₓ is hydrogen or methyl and d is 0 or 1;
   wherein each of R¹¹, R¹⁵, R^{15a}, R¹⁶, R¹⁷, R¹⁸ or R¹⁹, or any ring formed when R¹⁶ and R¹⁷ are linked, is optionally substituted with one or more R^{a};
R¹² is selected from one of the following options:
   (i) when R¹¹ is a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²,
         wherein:
         Rᵥ is hydrogen or methyl;
         d is 0 or 1; and
         Z¹² is —OR²⁰, R^{20a}, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rᵤ is hydrogen or methyl and e is 0, 1, 2 or 3;
         R^{20a} is (1-4C)alkyl, (3-7C)cycloalkyl, phenyl, 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ4-[4 to 8-membered heterocyclyl] or —(CHRᵤ)ₑ-[5 or 6 membered heteroaryl], wherein Rᵤ is hydrogen or methyl and e is 0, 1, 2 or 3;
         R²¹ and R²² are each independently selected from hydrogen, (1-6C)alkyl, (2-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[⁴ to 8-membered heterocyclyl] or —(CHR_{q})_{f}-[5 or 6 membered heteroaryl], wherein R_{q} is hydrogen or methyl and f is 0 or 1;
         or R²¹ and R²² are linked, such that, together with the nitrogen atom to which they are attached, they form a 4-8 membered heterocyclic ring;
         R²³ is hydrogen or (1-2C)alkyl;
         R²⁴ is (1-6C)alkyl, (2-6C)alkynyl, (3-7)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rp is hydrogen or methyl and g is 0 or 1;
   wherein each of R¹², R²⁰, R^{20a}, R²¹, R²², R²³ or R²⁴, or any ring formed when R²¹ and R²² are linked, is optionally substituted with one or more R^{a};
R¹³ is selected from cyano, halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein:
      Rₒ is hydrogen or methyl;
      h is 0 or 1; and
      Z¹³ is —OR²⁵, R^{25a}, —NR²⁶R²⁷, —C(O)NR²⁶R²⁷ or —NR²⁸C(O)R²⁹;
         wherein R²⁵ is (1-4C)alkyl, (3-7C)cycloalkyl, phenyl, 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0, 1 or 2;
         R^{25a} is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ and i are as defined above;
         R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
         or R²⁶ and R²⁷ are linked, such that, together with the nitrogen atom to which they are attached, they form a 4-8 membered heterocyclic ring;
         R²⁸ is hydrogen or (1-2C)alkyl;
         R²⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
   wherein each of R¹³, R²⁵, R^{25a}, R²⁶, R²⁷, R²⁸ or R²⁹ or any ring formed when R²⁶ and R²⁷ are linked, is optionally substituted with one or more R^{a};
R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen or methyl;
      wherein m is 0 or 1; and
      Z¹⁴ is —OR³⁰, R^{30a}, —NR³¹R³², —C(O)NR³¹R³² or —NR³³C(O)R³⁴;
         wherein R³⁰ is (1-4C)alkyl, (3-7C)cycloalkyl, a carbon-linked 4 to 8-membered heterocyclyl, a 5 or 6 membered heteroaryl, —(CHRⱼ)ₒ-(3-7C)cycloalkyl, —(CHRⱼ)ₒ-[4 to 8-membered heterocyclyl] or —(CHRⱼ)ₒ-[5 or 6 membered heteroaryl], wherein R; is hydrogen or methyl and o is 0 or 1;
         R^{30a} is (1-4C)alkyl, (3-7C)cycloalkyl, 4 to 8-membered heterocyclyl, a 5 or 6 membered heteroaryl, —(CHRⱼ)ₒ-(3-7C)cycloalkyl, —(CHRⱼ)ₒ-[⁴ to 8-membered heterocyclyl] or —(CHRⱼ)ₒ-[5 or 6 membered heteroaryl], wherein Rⱼ is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 8-membered heterocyclyl, a 5 or 6 membered heteroaryl, —(CHRᵢ)ₚ-(3-7C)cycloalkyl, —(CHRᵢ)ₚ-[4 to 8-membered heterocyclyl] or —(CHRᵢ)ₚ-[5 or 6 membered heteroaryl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen or (1-2C)alkyl;
         R³⁴ is (1-6C)alkyl, (2-6C)alkynyl, (3-7C)cycloalkyl, a carbon-linked 4 to 8-membered heterocyclyl, a 5 or 6 membered heteroaryl, —(CHRₕ)_{q}-(3-7C)cycloalkyl, —(CHRₕ)_{q}-[⁴ to 8-membered heterocyclyl] or —(CHRₕ)_{q}-[5 or 6 membered heteroaryl], wherein Rₕ is hydrogen or methyl and q is 0 or 1;
   wherein R¹⁴, R³⁰, R^{30a}, R³¹, R³², R³³ or R³⁴, or any ring formed when R³¹ and R³² are linked, is optionally substituted with one or more R^{a};
and wherein each R^{a} is independently selected from the group consisting of oxo, halogen, cyano, hydroxy, (1-4C)alkyl, or a group

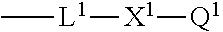

wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —N(R¹⁴)—C(O)—NR¹⁴—, —SO₂N(R¹)—, or —N(R¹⁴)SO₂—, where R¹⁴ is hydrogen or (1-2C)alkyl;
   Q¹ is selected from the group consisting of hydrogen, (1-4C)alkyl, (2-4C)alkenyl, (2-4C)alkynyl, or (3-6C)cycloalkyl;
wherein when Q¹ is (1-4C)alkyl, (2-4C)alkenyl, (2-4C)alkynyl, or (3-6C)cycloalkyl, Q¹ is optionally substituted with one or more Q^{a}, where each Q^{a} is independently selected from the group consisting of hydroxy, cyano, halogen, (1-3C)alkyl, (1-3C)alkoxy, (1-3C)haloalkyl, or (1-3C)haloalkoxy;
and wherein one of R¹² or R¹³ is optionally a group:

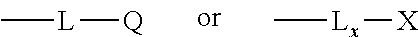

wherein L is a linker;
Lₓ is a linker;
X is a functional group; and
Q is a detection moiety.

In a further aspect, the present invention provides a probe compound of formula (I) defined herein, wherein R², R³, R⁴, R⁵, R⁶, Y₁, Y₂, A₁, A₂, A₃, A₄, R¹¹, R¹², R¹³ and R¹⁴ each have any one of the definitions provided hereinbefore, with the proviso that one of R¹² or R¹³ is a group:

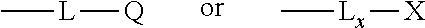

wherein L is a linker;
Lₓ is a linker;
X is a functional group; and
Q is a detection moiety.

In a particular group of compounds of the invention, R² is hydrogen.

In a further group of compounds of the invention, the compound is a compound of the formula (I-I) to (I-XLV) shown below, or pharmaceutically acceptable salt thereof:

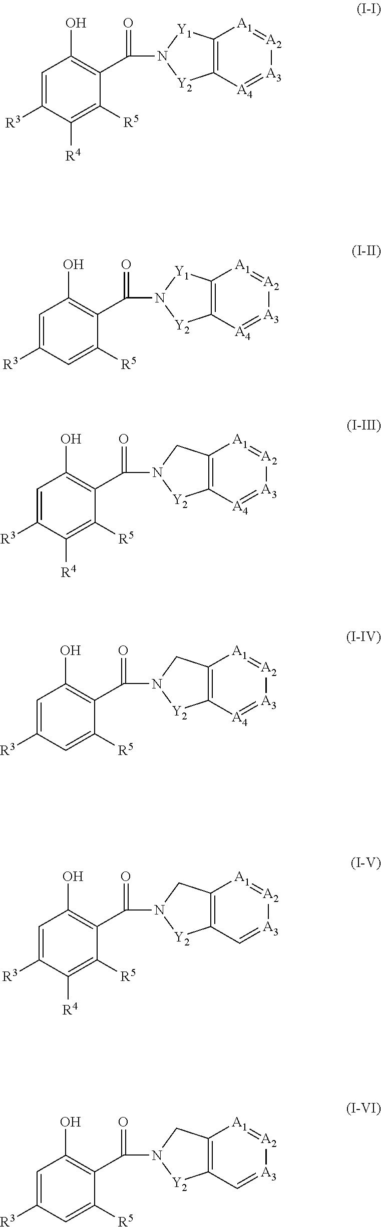

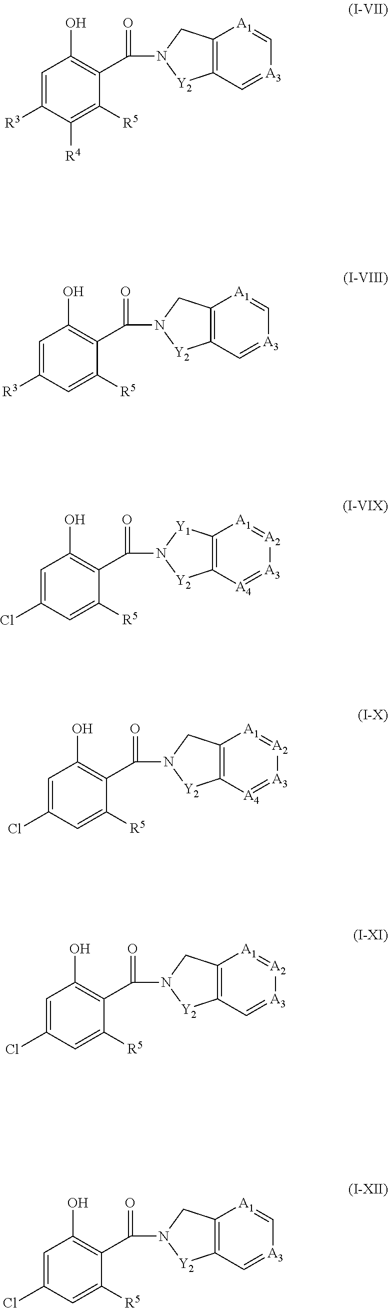

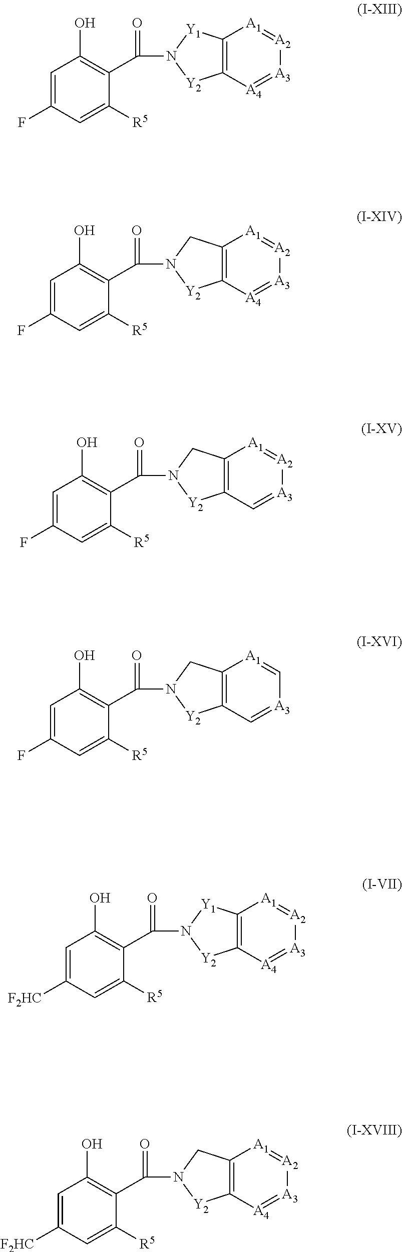

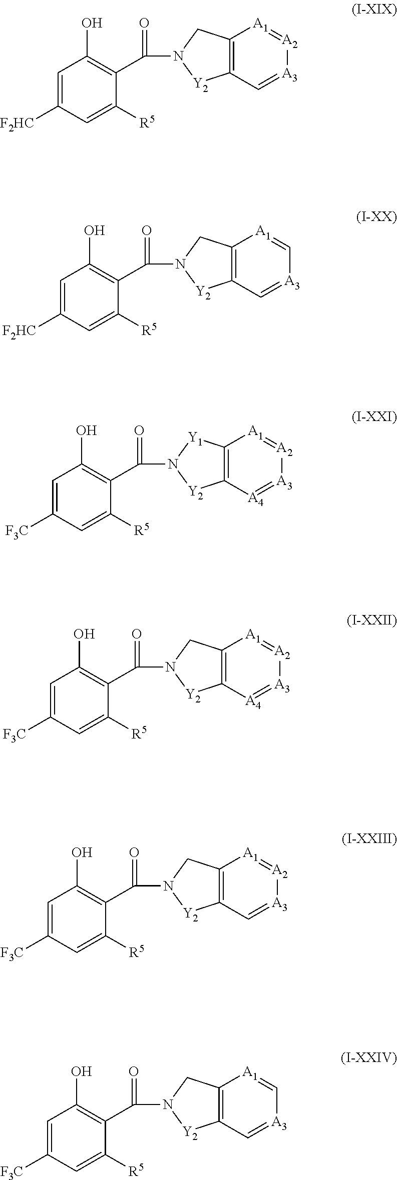

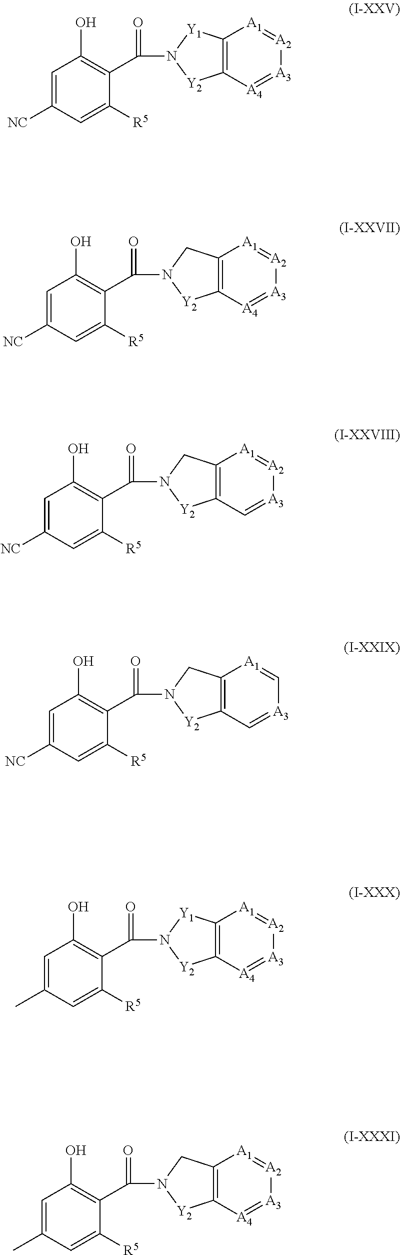

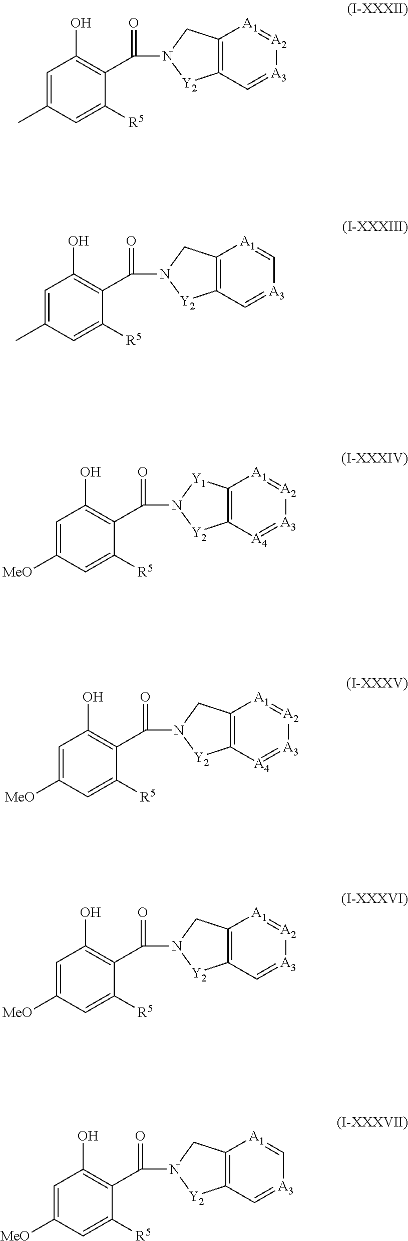

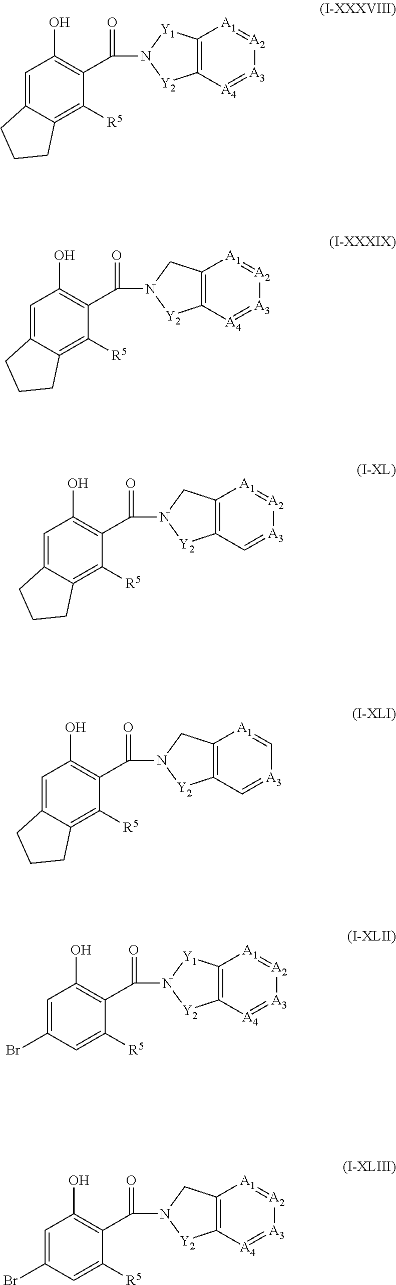

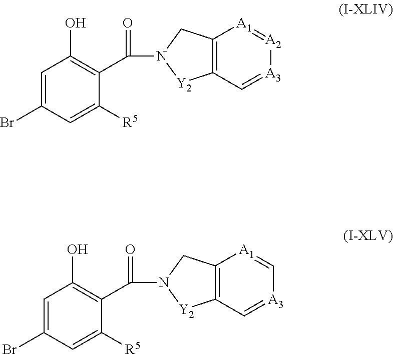

wherein R³, R⁴, R⁵, Y₁, Y₂, A₁, A₂, A₃, A₄, and any associated substituent groups each have any one of the definitions set out herein.

Particular compounds of the invention include, for example, compounds of the Formula (I), or any one of formulae (I-I) to (I-XLV) above, or pharmaceutically acceptable salts, hydrates and/or solvates thereof, wherein, unless otherwise stated, and where present, each of R², R³, R⁴, R⁵, Y₁, Y₂, A₁, A₂, A₃, A₄, L, Lₓ, X, Q and any associated substituent groups has any of the meanings defined hereinbefore or in any of paragraphs (1) to (161) hereinafter:
(1) R² is hydrogen;
(2) R² is fluoro;
(3) R⁴ is selected from hydrogen, fluoro, chloro or cyano;
(4) R⁴ is selected from hydrogen, fluoro or chloro;
(5) R⁴ is selected from hydrogen or fluoro;
(6) R⁴ is hydrogen;
(7) R⁴ is fluoro;
(8) R³ is selected from halo, cyano, (1-2C)alkyl, (1-2C)haloalkyl, (1-2C)hydroxyalkyl, (1-2C)alkoxy, (1-2C)haloalkoxy; or R³ and R⁴ are linked to form a fused 5- or 6-membered carbocyclic or heterocyclic ring, which is optionally further substituted by halo, hydroxy, cyano, (1-2C)alkyl, (1-2C)haloalkyl, (1-2C)alkoxy or (1-2C)haloalkoxy;
(9) R³ is selected from halo, cyano, (1-2C)alkyl, (1-2C)haloalkyl, (1-2C)hydroxyalkyl, (1-2C)alkoxy, (1-2C)haloalkoxy; or R³ and R⁴ are linked to form a fused 5- or 6-membered carbocyclic or heterocyclic ring, which is optionally further substituted by halo, hydroxy, cyano, (1-2C)alkyl, (1-2C)haloalkyl, (1-2C)alkoxy or (1-2C)haloalkoxy;
(10) R³ is selected from halo, cyano, (1-2C)alkyl, (1-2C)fluoroalkyl, (1-2C)alkoxy, (1-2C)fluoroalkoxy; or R³ and R⁴ are linked to form a fused 5- or 6-membered carbocyclic or heterocyclic ring, which is optionally further substituted by halo, hydroxy, cyano, (1-2C)alkyl, (1-2C)fluoroalkyl, (1-2C)alkoxy or (1-2C)fluoroalkoxy;
(11) R³ is selected from halo, cyano, methyl, fluoromethyl, di-fluoromethyl, trifluoromethyl, methoxy, di-fluoromethoxy or trifluoromethoxy; or R³ and R⁴ are linked to form a fused 5- or 6-membered carbocyclic or heterocyclic ring, which is optionally further substituted by halo, cyano, methyl, fluoromethyl, di-fluoromethyl, trifluoromethyl, methoxy, di-fluoromethoxy or trifluoromethoxy;
(12) R³ is selected from halo, cyano, methyl, di-fluoromethyl, trifluoromethyl, or methoxy; or R³ and R⁴ are linked to form a fused 5- or 6-membered carbocyclic or heterocyclic ring, which is optionally further substituted by halo, cyano, methyl, di-fluoromethyl, trifluoromethyl or methoxy;
(13) R³ is selected from halo, cyano, methyl, di-fluoromethyl, trifluoromethyl, or methoxy; or R³ and R⁴ are linked to form a fused 5- or 6-membered carbocyclic or heterocyclic ring;
(14) R³ is selected from halo, cyano, methyl, di-fluoromethyl, trifluoromethyl, or methoxy; or R³ and R⁴ are linked to form a fused 5- or 6-membered cycloalkyl ring;
(15) R⁴ is hydrogen and R³ is selected from halo, cyano, (1-2C)alkyl, (1-2C)haloalkyl, (1-2C)hydroxyalkyl, (1-2C)alkoxy, (1-2C)haloalkoxy; or R³ and R⁴ are linked to form a fused 5- or 6-membered carbocyclic or heterocyclic ring, which is optionally further substituted by halo, hydroxy, cyano, (1-2C)alkyl, (1-2C)haloalkyl, (1-2C)alkoxy or (1-2C)haloalkoxy;
(16) R⁴ is hydrogen and R³ is selected from halo, cyano, (1-2C)alkyl, (1-2C)haloalkyl, (1-2C)hydroxyalkyl, (1-2C)alkoxy, (1-2C)haloalkoxy; or R³ and R⁴ are linked to form a fused 5- or 6-membered carbocyclic or heterocyclic ring, which is optionally further substituted by halo, hydroxy, cyano, (1-2C)alkyl, (1-2C)haloalkyl, (1-2C)alkoxy or (1-2C)haloalkoxy;
(17) R⁴ is hydrogen and R³ is selected from halo, cyano, (1-2C)alkyl, (1-2C)fluoroalkyl, (1-2C)alkoxy, (1-2C)fluoroalkoxy; or R³ and R⁴ are linked to form a fused 5- or 6-membered carbocyclic or heterocyclic ring, which is optionally further substituted by halo, hydroxy, cyano, (1-2C)alkyl, (1-2C)fluoroalkyl, (1-2C)alkoxy or (1-2C)fluoroalkoxy;
(18) R⁴ is hydrogen and R³ is selected from halo, cyano, methyl, fluoromethyl, di-fluoromethyl, trifluoromethyl, methoxy, di-fluoromethoxy or trifluoromethoxy; or R³ and R⁴ are linked to form a fused 5- or 6-membered carbocyclic or heterocyclic ring, which is optionally further substituted by halo, cyano, methyl, fluoromethyl, di-fluoromethyl, trifluoromethyl, methoxy, di-fluoromethoxy or trifluoromethoxy;
(19) R⁴ is hydrogen and R³ is selected from halo, cyano, methyl, di-fluoromethyl, trifluoromethyl, or methoxy; or R³ and R⁴ are linked to form a fused 5- or 6-membered carbocyclic or heterocyclic ring, which is optionally further substituted by halo, cyano, methyl, di-fluoromethyl, trifluoromethyl or methoxy;
(20) R⁴ is hydrogen and R³ is selected from halo, cyano, methyl, di-fluoromethyl, trifluoromethyl, or methoxy; or R³ and R⁴ are linked to form a fused 5- or 6-membered carbocyclic or heterocyclic ring;
(21) R⁴ is hydrogen and R³ is selected from halo, cyano, methyl, di-fluoromethyl, trifluoromethyl, or methoxy; or R³ and R⁴ are linked to form a fused 5- or 6-membered cycloalkyl ring;
(22) R⁵ is —OR⁶, wherein R⁶ is as defined above or in any one of paragraphs (25) to (36) hereinafter;
(23) R⁵ is —OR⁶, wherein R⁶ is as defined above or in any one of paragraphs (33), (34), (35) or (36) hereinafter;
(24) R⁵ is —OR⁶, wherein R⁶ is as defined above or in paragraph (36) hereinafter;
(25) R⁶ is (1-6C)alkyl, (3-6C)cycloalkyl, or a 4- to 6-membered heterocyclyl ring comprising one heteroatom selected from N, O or S, each of which is optionally substituted by a substituent selected from the group consisting of hydroxy, cyano, halogen, amino, amido (1-2C)alkyl, (1-2C)alkoxy, (1-2C)haloalkyl, or (1-2C)haloalkoxy;
   or a group having a structure according to formula (A) shown below:

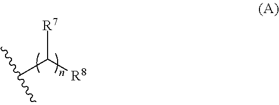

wherein
   R⁷ is hydrogen or (1-3C)alkyl;
   n is 1 or 2;
   R⁸ is (3-7C)cycloalkyl, phenyl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
(26) R⁶ is (1-6C)alkyl, (3-6C)cycloalkyl, or a 4- to 6-membered heterocyclyl ring comprising one heteroatom selected from N, O or S,
   or a group having a structure according to formula (A) shown below:

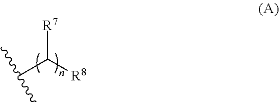

wherein
   R⁷ is hydrogen or (1-3C)alkyl;
   n is 1 or 2;
   R⁸ is (3-8C)cycloalkyl, phenyl, 4- to 6-membered heterocyclyl, 5- or 6-membered heteroaryl, or 9- or 10-membered fused bicyclic heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy, and wherein any 4- to 6-membered heterocyclyl optionally comprises 1 or 2 additional bridging atoms;
(27) R⁶ is (1-6C)alkyl or a group having a structure according to formula (A) shown below:

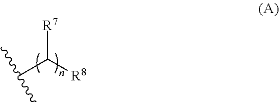

wherein
   R⁷ is hydrogen or (1-3C)alkyl;
   n is 1 or 2;
   R⁸ is (3-6C)cycloalkyl, phenyl, 4- to 6-membered heterocyclyl, 5- or 6-membered heteroaryl, or 9- or 10-membered fused bicyclic heteroaryl, each of which is optionally substituted with one or more R⁹; where each R⁹ is independently selected from the group consisting of hydroxy, cyano, halogen, (1-2C)alkyl, (1-2C)alkoxy, (1-2C)haloalkyl, or (1-2C)haloalkoxy, and wherein any 4- to 6-membered heterocyclyl optionally comprises 1 or 2 additional bridging atoms;
(28) R⁶ is (1-4C)alkyl or a group having a structure according to formula (A) shown below:

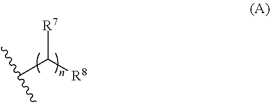

wherein
   R⁷ is hydrogen or (1-2C)alkyl;
   n is 1 or 2;
   R⁸ is (3-6C)cycloalkyl, phenyl, 4- to 6-membered heterocyclyl, 5- or 6-membered heteroaryl, or 9- or 10-membered fused bicyclic heteroaryl, each of which is optionally substituted with one or more R⁹; where each R⁹ is independently selected from the group consisting of hydroxy, cyano, halogen, (1-2C)alkyl, (1-2C)alkoxy, (1-2C)haloalkyl, or (1-2C)haloalkoxy, and wherein any 4- to 6-membered heterocyclyl optionally comprises 1 additional bridging atom;
(29) R⁶ is (1-4C)alkyl or a group having a structure according to formula (A) shown below:

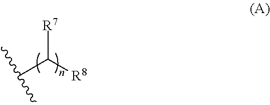

wherein
   R⁷ is hydrogen or methyl;
   n is 1;
   R⁸ is (3-6C)cycloalkyl, phenyl, 4- to 6-membered heterocyclyl, 5- or 6-membered heteroaryl, or 9- or 10-membered fused bicyclic heteroaryl, each of which is optionally substituted with one or more R⁹; where each R⁹ is independently selected from the group consisting of hydroxy, cyano, halogen, (1-2C)alkyl, (1-2C)alkoxy, (1-2C)haloalkyl, or (1-2C)haloalkoxy, and wherein any 4- to 6-membered heterocyclyl optionally comprises 1 additional bridging;
(30) R⁶ is (1-3C)alkyl or a group having a structure according to formula (A) shown below:

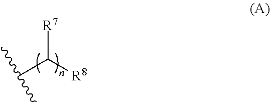

wherein
   R⁷ is hydrogen or methyl;
   n is 1;
   R⁸ is (3-6C)cycloalkyl, phenyl, 6-membered heterocyclyl, 6-membered heteroaryl, or 9- or 10-membered fused bicyclic heteroaryl, each of which is optionally substituted with one or more R⁹; where each R⁹ is independently selected from the group consisting of halogen, methyl, methoxy, trifluoromethyl, or trifluoromethoxy;
(31) R⁶ is methyl, ethyl or a group having a structure according to formula (A) shown below:

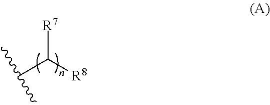

wherein
   R⁷ is hydrogen or methyl;
   n is 1;
   R⁸ is (3-6C)cycloalkyl, phenyl, 6-membered heterocyclyl, 6-membered heteroaryl, or 9- or 10-membered fused bicyclic heteroaryl, each of which is optionally substituted with one or more R⁹; where each R⁹ is independently selected from the group consisting of halogen, methyl, methoxy, trifluoromethyl, or trifluoromethoxy;
(32) R⁶ is methyl or a group having a structure according to formula (A) shown below:

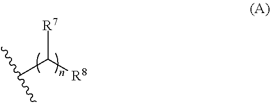

wherein
   R⁷ is hydrogen or methyl;
   n is 1;
   R⁸ is cyclopropyl, cyclohexyl, phenyl, 6-membered heterocyclyl, pyridyl, benzimidazole or quinoline, each of which is optionally substituted with one or more R⁹; where each R⁹ is independently selected from the group consisting of halogen, methyl, methoxy, trifluoromethyl, or trifluoromethoxy;
(33) R⁶ is methyl or a group having a structure according to formula (A) shown below:

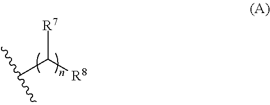

wherein
   R⁷ is hydrogen or methyl;
   n is 1;
   R⁸ is cyclopropyl, cyclohexyl, phenyl, pyridyl, benzimidazole or quinoline, each of which is optionally substituted with one or more R⁹; where each R⁹ is independently selected from the group consisting of fluoro, chloro, methyl, or methoxy;
(34) R⁶ is methyl or a group having a structure according to formula (A) shown below:

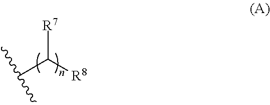

wherein
   R⁷ is hydrogen or methyl;
   n is 1;
   R⁸ is cyclopropyl or phenyl, each of which is optionally substituted with one or more R⁹; where each R⁹ is independently selected from the group consisting of fluoro, chloro, methyl, or methoxy;
(35) R⁶ is methyl or a group having a structure according to formula (A) shown below:

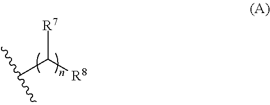

wherein
   R⁷ is hydrogen or methyl;
   n is 1;
   R⁸ is cyclopropyl or phenyl;
(36) R⁶ is a group having a structure according to formula (A) shown below:

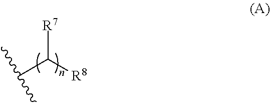

wherein
   R⁷ is hydrogen or methyl;
   n is 1;
   R⁸ is phenyl;
(37) Y₁ is —CH₂—;
(37a) Y₁ is a bond, —CH₂— or —CH₂—CH₂—;
(38) Y₁ is —C(═O)—;
(39) Y₂ is —CH₂—, —CHR_{y2a}—, —CH₂—CH₂—, —CH₂—CHR_{y2b}—, or —CHR_{y2a}—CH₂—; where R_{y2a} is selected from halo, cyano, methyl, CF₃ or hydroxymethyl, and R_{y2b} is selected from halo, cyano, hydroxy, methyl, methoxy, CF₃, —OCF₃ or hydroxymethyl, provided that when Y₁ is a bond, Y₂ is —CH₂—CH₂—, —CH₂—CHR_{y2b}—, or —CHR_{y2a}—CH₂—;
(40) Y₂ is —CH₂—, —CHR_{y2a}—, —CH₂—CH₂—, —CH₂—CHR_{y2b}—, or —CHR_{y2a}—CH₂—; where R_{y2a} is selected from halo, methyl or hydroxymethyl, and R_{y2b} is selected from halo, cyano, hydroxy, methyl, methoxy, CF₃, —OCF₃ or hydroxymethyl, provided that when Y₁ is a bond, Y₂ is —CH₂—CH₂—, —CH₂—CHR_{y2b}—, or —CHR_{y2a}—CH₂—;
(41) Y₂ is —CH₂—, —CH₂—CH₂—, or —CH₂—CHR_{y2b}—; where R_{y2b} is selected from halo, cyano, hydroxy, methyl, methoxy, CF₃, —OCF₃ or hydroxymethyl, provided that when Y₁ is a bond, Y₂ is —CH₂—CH₂— or —CH₂—CHR_{y2b}—;
(42) Y₂ is —CH₂— or —CH₂—CH₂—, provided that when Y₁ is a bond, Y₂ is —CH₂—CH₂—;
(43) Y₂ is —CH₂—;
(44) Y₂ is —CH₂—CH₂—;
(45) A₁ is selected from CH or CR¹¹;
(46) A₁ is CH;
(47) A₁ is CR¹¹;
(48) A₂ is selected from CH or CR¹²
(49) A₂ is N;
(50) A₂ is CH;
(51) A₂ is CR¹²;
(52) A₃ is selected from CH or CR¹³;
(53) A₃ is N;
(54) A₃ is CH;
(55) A₃ is CR¹³;
(56) A₄ is selected from CH or CR¹⁴;
(57) A₄ is N;
(58) A₄ is CH;
(59) A₄ is CR¹⁴;
(60) A₁ is selected from N, CH or CR¹¹, wherein R¹¹ is as defined hereinbefore or in any one of paragraphs (68) to (86) hereinafter;
   A₂ is selected from N, CH or CR¹², wherein R¹² is as defined hereinbefore or in any one of paragraphs (87) to (96) hereinafter;
   A₃ is selected from N, CH or CR¹³, wherein R¹³ is as defined hereinbefore or in any one of paragraphs (97) to (101) hereinafter;
   A₄ is selected from N, CH or CR¹⁴, wherein R¹⁴ is as defined hereinbefore or in any one of paragraphs (102) to (105) hereinafter;
   with the proviso that only one of A₁, A₂, A₃ or A₄ can be N;
(61) A₁ is selected from CH or CR¹¹, wherein R¹¹ is as defined hereinbefore or in paragraph (74) hereinafter;
   A₂ is selected from N, CH or CR¹², wherein R¹² is as defined hereinbefore or in (89) hereinafter;
   A₃ is selected from N, CH or CR¹³, wherein R¹³ is as defined hereinbefore or in paragraph (98) hereinafter;
   A₄ is selected from N, CH or CR¹⁴, wherein R¹⁴ is as defined hereinbefore or in paragraph (102) hereinafter;
   with the proviso that only one of A₂, A₃ or A₄ can be N;
(62) A₁ is CR¹¹, wherein R¹¹ is as defined hereinbefore or in paragraph (80) hereinafter;
   A₂ is selected from N, CH or CR¹², wherein R¹² is as defined hereinbefore or in paragraph (91) hereinafter;
   A₃ is selected from N, CH or CR¹³, wherein R¹³ is as defined hereinbefore or in paragraph (99) hereinafter;
   A₄ is selected from N, CH or CR¹⁴, wherein R¹⁴ is as defined hereinbefore or in paragraph (103) hereinafter;
   with the proviso that only one of A₂, A₃ or A₄ can be N;
(63) A₁ is CR¹¹, wherein R¹¹ is as defined hereinbefore or in paragraph (68) hereinafter;
   A₂ is selected from N or CH;
   A₃ is selected from N, CH or CR¹³; wherein R¹³ is as defined hereinbefore or in paragraph (97) hereinafter;
   A₄ is selected from N or CH;
   with the proviso that only one of A₂, A₃ or A₄ can be N;
(64) A₁ is CR¹¹, wherein R¹¹ is as defined hereinbefore or in paragraph (74) hereinafter;
   A₂ is CH;
   A₃ is CH or CR¹³, wherein R¹³ is as defined hereinbefore or in paragraph (99) hereinafter;
   A₄ is CH;
(65) A₁ is CR¹¹, wherein R¹¹ is as defined hereinbefore or in paragraph (80) hereinafter;
   A₂ is CH;
   A₃ is CH;
   A₄ is CH;
(66) A₁ is CH;
   A₂ is CR¹², wherein R¹² is as defined hereinbefore or in paragraph (87) hereinafter;
   A₃ is CH;
   A₄ is CH;
(67) A₁ is CH;
   A₂ is CH;
   A₃ is CR¹³, wherein R¹³ is as defined hereinbefore or in paragraph (97) hereinafter;
   A₄ is CH;
(68) R¹¹ is cyano, halo, or (1-2C)alkyl optionally substituted by one or more halo or (1-2C)alkoxy;
   or —(CHR_{z})ₐ—Z¹¹,
      wherein:
      R_{z} is hydrogen or methyl;
      a is 0 or 1; and
      Z¹¹ is —OR¹⁵, —NR¹⁶R¹⁷, —C(O)NR¹⁶R¹⁷ or —NR¹⁸C(O)R¹⁹;
         wherein
         R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
         R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1;
         or R¹⁶ and R¹⁷ are linked, such that, together with the nitrogen atom to which they are attached, they form a 4-6 membered heterocyclic ring;
         R¹⁸ is hydrogen or (1-2C)alkyl;
         R¹⁹ is (1-6C)alkyl, (3-6C)cycloalkyl, phenyl, a carbon-linked 4 to 6-membered heterocyclyl, a 5 or 6 membered heteroaryl, —(CHRₓ)_{d}-(3-6C)cycloalkyl, —(CHRₓ)_{d}-phenyl, —(CHRₓ)_{d}-[4 to 6-membered heterocyclyl] or —(CHRₓ)_{d}-[5 or 6 membered heteroaryl];
         wherein Rₓ is hydrogen or methyl and d is 0 or 1;
   wherein each of R¹¹, R¹⁵, R¹⁶, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (106) to (114) hereinafter;
(69) R¹¹ is cyano, halo or (1-2C)alkyl optionally substituted by one or more R^{a}; or

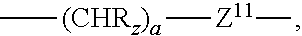

wherein R_{z} is hydrogen or methyl;
wherein a is 0 or 1; and
Z¹¹ is —OR¹⁵, —NR¹⁶R¹⁷, —C(O)NR¹⁶R¹⁷ or —NR¹⁸C(O)R¹⁹;
wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
or R¹⁶ and R¹⁷ are linked, such that, together with the nitrogen atom to which they are attached, they form a 4-6 membered heterocyclic ring;
R¹⁸ is hydrogen or (1-2C)alkyl;
R¹⁹ is (1-6C)alkyl, (3-6C)cycloalkyl, phenyl, a carbon-linked 4 to 6-membered heterocyclyl, a 5 or 6 membered heteroaryl, —(CHRₓ)_{d}-(3-60)cycloalkyl, —(CHRₓ)_{d}-phenyl, —(CHRₓ)_{d}-[4 to 6-membered heterocyclyl] or —(CHRₓ)_{d}-[5 or 6 membered heteroaryl];
wherein Rₓ is hydrogen or methyl and d is 0 or 1;
wherein each of R¹⁵, R¹⁶, R¹⁷, R¹⁸ or R¹⁹, or any ring formed when R¹⁶ and R¹⁷ are linked, is optionally substituted with one or more R^{a};
wherein R^{a} is as defined hereinbefore or in any one of paragraphs (106) to (114) hereinafter;
(70) R¹¹ is cyano, fluoro, chloro or (1-2C)alkyl optionally substituted by one or more fluoro, chloro or (1-2C)alkoxy;
   or —(CHR_{z})ₐ—Z¹¹,
      wherein R_{z} is hydrogen or methyl;
      wherein a is 0 or 1; and
      Z¹¹ is —OR¹⁵, —NR¹⁶R¹⁷, —C(O)NR¹⁶R¹⁷ or —NR¹⁸C(O)R¹⁹;
         wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
         R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl,
         (CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
         or R¹⁶ and R¹⁷ are linked, such that, together with the nitrogen atom to which they are attached, they form a 4-6 membered heterocyclic ring;
         R¹⁸ is hydrogen or (1-2C)alkyl;
         R¹⁹ is (1-6C)alkyl, (3-6C)cycloalkyl, phenyl, a carbon-linked 4 to 6-membered heterocyclyl, a 5 or 6 membered heteroaryl, or —(CHRₓ)_{d}-(3-6C)cycloalkyl;
         wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (106) to (114) hereinafter;
(71) R¹¹ is cyano or —(CHR_{z})ₐ—Z¹¹,
   wherein R_{z} is hydrogen or methyl;
   wherein a is 0 or 1; and
   Z¹¹ is —OR¹⁵, —NR¹⁶R¹⁷ or —NR¹⁸C(O)R¹⁹;
      wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
      or R¹⁶ and R¹⁷ are linked, such that, together with the nitrogen atom to which they are attached, they form a 4-6 membered heterocyclic ring;
      R¹⁸ is hydrogen or (1-2C)alkyl;
      R¹⁹ is (1-6C)alkyl, (3-6C)cycloalkyl, phenyl, a carbon-linked 4 to 6-membered heterocyclyl, a 5 or 6 membered heteroaryl, or —(CHRₓ)_{d}-(3-6C)cycloalkyl;
      wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (106) to (114) hereinafter;
(72) R¹¹ is —(CHR_{z})ₐ—Z¹¹,
   wherein R_{z} is hydrogen;
   wherein a is 0 or 1; and
   Z¹¹ is —OR¹⁵, —NR¹⁶R¹⁷ or —NR¹⁸C(O)R¹⁹;
      wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl,
      (CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
      or R¹⁶ and R¹⁷ are linked, such that, together with the nitrogen atom to which they are attached, they form a 4-6 membered heterocyclic ring;
      R¹⁸ is hydrogen or (1-2C)alkyl;
      R¹⁹ is (1-6C)alkyl, (3-6C)cycloalkyl, phenyl, a carbon-linked 4 to 6-membered heterocyclyl, or a 5 or 6 membered heteroaryl;
      wherein Rₓ is hydrogen or methyl and d is 0 or 1;
   wherein each of R¹⁵, R¹⁶, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (106) to (114) hereinafter;
(73) R¹¹ is —(CHR_{z})ₐ—Z¹¹,
   wherein R_{z} is hydrogen or methyl;
   wherein a is 0 or 1; and
   Z¹¹ is —OR¹⁵, —NR¹⁶R¹⁷ or —NR¹⁸C(O)R¹⁹;
      wherein R¹⁵ is (1-4C)alkyl, (3-6C)cycloalkyl, a carbon-linked 4 to 6-membered heterocyclyl, 5 or 6 membered heteroaryl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      wherein R¹⁶ is hydrogen and R¹⁷ is selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1;
      or R¹⁶ and R¹⁷ are linked, such that, together with the nitrogen atom to which they are attached, they form a 4-6 membered heterocyclic ring;
      R¹⁸ is hydrogen or (1-2C)alkyl;
      R¹⁹ is (1-6C)alkyl, (3-6C)cycloalkyl, phenyl, a carbon-linked 4 to 6-membered heterocyclyl, or a 5 or 6 membered heteroaryl;
      wherein Rₓ is hydrogen or methyl and d is 0 or 1;
   wherein each of R¹⁵, R¹⁷, R¹⁸ or R¹⁹, or any ring formed when R¹⁶ and R¹⁷ are linked, is optionally substituted with one or more R^{a};
   wherein R^{a} is as defined hereinbefore or in any one of paragraphs (106) to (114) hereinafter;
(74) R¹¹ is —(CHR_{z})ₐ—Z¹¹,
   wherein R_{z} is hydrogen or methyl;
   wherein a is 0 or 1; and
   Z¹¹ is —OR¹⁵ or —NR¹⁶R¹⁷;
      wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      wherein R¹⁶ is hydrogen and R¹⁷ is selected from hydrogen, (1-6C)alkyl, (3-6C)cycloalkyl, phenyl, a carbon-linked 4 to 8-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{w} is hydrogen or methyl and c is 0 or 1;
   wherein each of R¹⁵ or R¹⁷ is optionally substituted with one or more R^{a};
   wherein R^{a} is as defined hereinbefore or in any one of paragraphs (106) to (114) hereinafter;
(75) R¹¹ is —(CHR_{z})ₐ—Z¹¹,
   wherein R_{z} is hydrogen or methyl;
   wherein a is 0 or 1; and
   Z¹¹ is —OR¹⁵ or —NR¹⁶R¹⁷
      wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      wherein R¹⁶ is hydrogen and R¹⁷ is selected from (3-6C)cycloalkyl, phenyl, a carbon-linked 4 to 8-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{w} is hydrogen or methyl and c is 0 or 1;
   wherein each of R¹⁵ or R¹⁷ is optionally substituted with one or more R^{a};
   wherein R^{a} is as defined hereinbefore or in any one of paragraphs (106) to (114) hereinafter;
(76) R¹¹ is —(CHR_{z})ₐ—Z¹¹,
   wherein R_{z} is hydrogen or methyl;
   wherein a is 0 or 1; and
   Z¹¹ is —OR¹⁵ or —NR¹⁶R¹⁷;
      wherein R¹⁵ is (1-4C)alkyl, (3-6C)cycloalkyl, a carbon-linked 4 to 6-membered heterocyclyl, 5 or 6 membered heteroaryl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
      wherein R¹⁶ is hydrogen and R¹⁷ is selected from (3-6C)cycloalkyl, phenyl, a carbon-linked 4 to 6-membered heterocyclyl, a 5 or 6 membered heteroaryl;
   wherein each of R¹⁵ or R¹⁷ is optionally substituted with one or more R^{a};
   wherein R^{a} is as defined hereinbefore or in any one of paragraphs (106) to (114) hereinafter;
(77) R¹¹ is —(CHR_{z})ₐ—Z¹¹,
   wherein R_{z} is hydrogen or methyl;
   wherein a is 0 or 1; and
   Z¹¹ is —OR¹⁵ or —NR¹⁶R¹⁷;
      wherein R¹⁵ is (1-4C)alkyl, (3-6C)cycloalkyl, a carbon-linked 4 to 6-membered heterocyclyl or a 5 or 6 membered heteroaryl;
      wherein R¹⁶ is hydrogen and R¹⁷ is selected from (3-6C)cycloalkyl, phenyl, a carbon-linked 4 to 6-membered heterocyclyl, a 5 or 6 membered heteroaryl, —CH₂-(3-6C)cycloalkyl, —CH₂-phenyl, —CH₂-[4 to 8-membered heterocyclyl] or —CH₂-[5 or 6 membered heteroaryl];
   wherein each of R¹⁵ or R¹⁷ is optionally substituted with one or more R^{a};
   wherein R^{a} is as defined hereinbefore or in any one of paragraphs (106) to (114) hereinafter;
(78) R¹¹ is —(CHR_{z})ₐ—Z¹¹,
   wherein R_{z} is hydrogen or methyl;
   wherein a is 0 or 1; and
   Z¹¹ is —OR¹⁵ or —NR¹⁶R¹⁷;
      wherein R¹⁵ is (1-4C)alkyl, (4-6C)cycloalkyl, a carbon-linked 5 or 6-membered heterocyclyl or a 5 or 6 membered heteroaryl;
      wherein R¹⁶ is hydrogen and R¹⁷ is selected from (4-6C)cycloalkyl, phenyl, a carbon-linked 5 or 6-membered heterocyclyl, a 5 or 6 membered heteroaryl, —CH₂-(4-6C)cycloalkyl, —CH₂-phenyl, —CH₂-[4 to 6-membered heterocyclyl] or CH₂-[5 or 6 membered heteroaryl];
   wherein each of R¹⁵ or R¹⁷ is optionally substituted with one or more R^{a};
   wherein R^{a} is as defined hereinbefore or in any one of paragraphs (106) to (114) hereinafter;
(79) R¹¹ is —(CHR_{z})ₐ—Z¹¹,
   wherein R_{z} is hydrogen;
   wherein a is 0 or 1; and
   Z¹¹ is —NR¹⁶R¹⁷;
      wherein R¹⁶ is hydrogen and R¹⁷ is selected from (3-6C)cycloalkyl, phenyl, a carbon-linked 4 to 8-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1;
   wherein R¹⁷ is optionally substituted with one or more R^{a};
   wherein R^{a} is as defined hereinbefore or in any one of paragraphs (106) to (114) hereinafter;
(80) R¹¹ is —(CHR_{z})ₐ—Z¹¹,
   wherein R_{z} is hydrogen;
   wherein a is 0 or 1; and
   Z¹¹ is: —OR¹⁵, wherein R¹⁵ is (1-4C)alkyl, optionally substituted by one or more R^{a};
      wherein R^{a} is as defined hereinbefore or in any one of paragraphs (106) to (114) hereinafter; or
   —NHR¹⁷, wherein R¹⁷ is selected from:
   (i) a group of the formula (A), (B), (C) or (D):

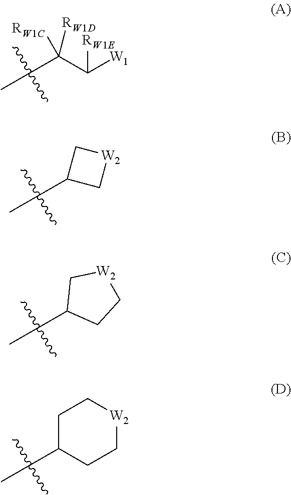

wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen or methyl, or R_{W1C} and R_{W1D} or R_{W1D} and R_{W1E} or R_{W1E} and W₁ are linked such that, together with the carbon atoms to which they are attached, they form a (4-6C)cycloalkyl ring, a 5 or 6 membered heterocyclyl ring, or a 5 or 6-membered heteroaryl, each of which is optionally substituted by one or more R^{a};
W₂ is selected from O, NR_{w2}, S or S(O)₂ where R_{w2} is selected from hydrogen, (1-4C)alkyl or (2-4C)alkanoyl;
and each of formulae (B), (C) and (D) is optionally substituted by one or more R^{a};
wherein R^{a} is as defined hereinbefore or in any one of paragraphs (106) to (114) hereinafter;
(ii) a bridged version of formulae (C) and (D) above, optionally selected from one of formulae (E) to (J) below:

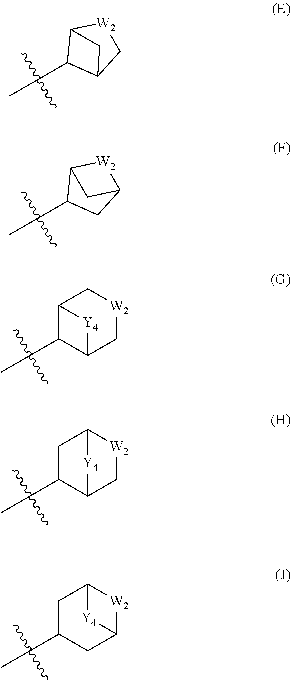

wherein Y₄ is selected from —CH₂—, —CH₂CH₂—,
and each of formulae (E), (F), (G), (H) and (J) is optionally substituted by one or more R^{a};
wherein R^{a} is as defined hereinbefore or in any one of paragraphs (106) to (114) hereinafter;
(iii) a group of the formula (K), (L) or (M) below:

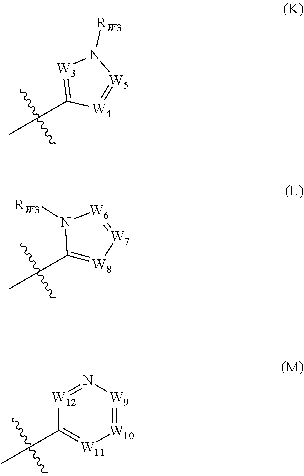

wherein:
R_{W3} is selected from hydrogen or (1-2C)alkyl;
W₃, W₄ and W₅ are selected from N or CR_{w4} where R_{w4} is hydrogen, halo, (1-2C)alkyl or (1-2C)alkoxy;
one of W₆ and W₇ is N and the other is CR_{w5} and W₈ is N or CR_{w5}, where R_{w5} is hydrogen, halo, (1-2C)alkyl or (1-2C)alkoxy;
W₉, W₁₀, W₁₁ and W₁₂ are selected from N or CR_{w6} where R_{w6} is hydrogen, halo, (1-2C)alkyl or (1-2C)alkoxy; with the proviso that only one or two of W₉, W₁₀, W₁₁ and W₁₂ are N;
(81) R¹¹ is —(CHR_{z})ₐ—Z¹¹,
   wherein R_{z} is hydrogen;
   wherein a is 0 or 1; and
   Z¹¹ is: —OR¹⁵, wherein R¹⁵ is (1-4C)alkyl; or
      —NHR¹⁷, wherein R¹⁷ is selected from:
      (i) a group of the formula (A), (B), (C) or (D):

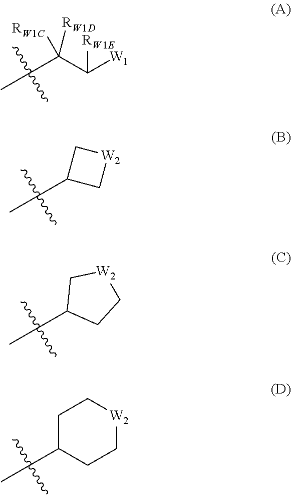

wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen or methyl, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2}, S or S(O)₂ where R_{w2} is selected from hydrogen, (1-4C)alkyl or (2-4C)alkanoyl;
and each of formulae (B), (C) and (D) is optionally substituted by one or more R^{a};
wherein R^{a} is as defined hereinbefore or in any one of paragraphs (106) to (114) hereinafter;
(ii) a bridged version of formulae (C) and (D) above, optionally selected from one of formulae (E) to (J) below:

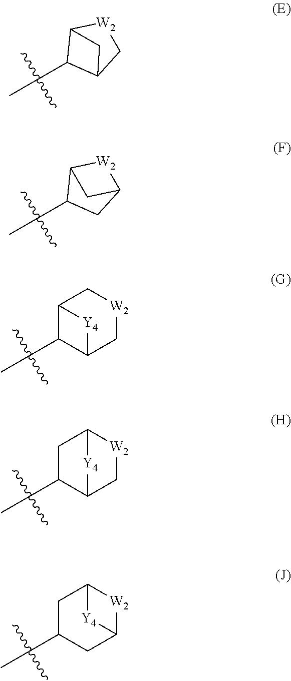

wherein Y₄ is selected from —CH₂—, —CH₂CH₂—,
and each of formulae (E), (F), (G), (H) and (J) is optionally substituted by one or more R^{a};
wherein R^{a} is as defined hereinbefore or in any one of paragraphs (106) to (114) hereinafter;
(iii) a group of the formula (K), (L) or (M) below:

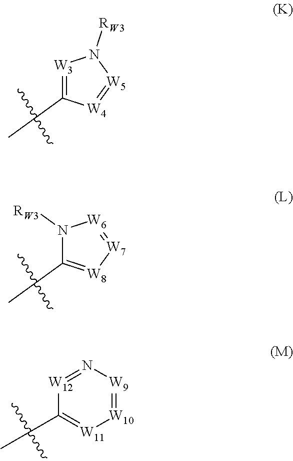

wherein:
R_{W3} is selected from hydrogen or (1-2C)alkyl;
W₃, W₄ and W₅ are selected from N or CR_{w4} where R_{w4} is hydrogen, halo, (1-2C)alkyl or (1-2C)alkoxy;
one of W₆ and W₇ is N and the other is CR_{w5} and W₈ is N or CR_{w5}, where R_{w5} is hydrogen, halo, (1-2C)alkyl or (1-2C)alkoxy;
W₉, W₁₀, W₁₁ and W₁₂ are selected from N or CR_{w6} where R_{w6} is hydrogen, halo, (1-2C)alkyl or (1-2C)alkoxy; with the proviso that only one or two of W₉, W₁₀, W₁₁ and W₁₂ are N;
(82) R¹¹ is —(CHR_{z})ₐ—Z¹¹,
   wherein R_{z} is hydrogen;
   wherein a is 0 or 1; and
   Z¹¹ is: —OR¹⁵, wherein R¹⁵ is (1-3C)alkyl; or
      —NHR¹⁷, wherein R¹⁷ is selected from:
         (i) a group of the formula (A), (B), (C) or (D):

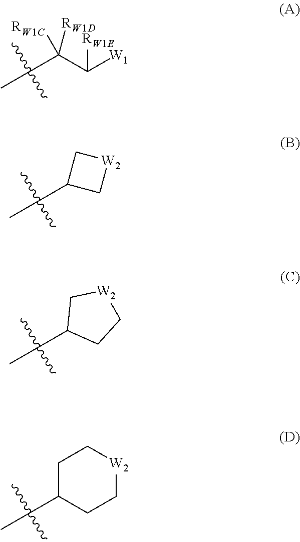

wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined hereinbefore or in any one of paragraphs (106) to (114) hereinafter;
(ii) a bridged version of formulae (C) and (D) above, optionally selected from one of formulae (E) to (J) below:

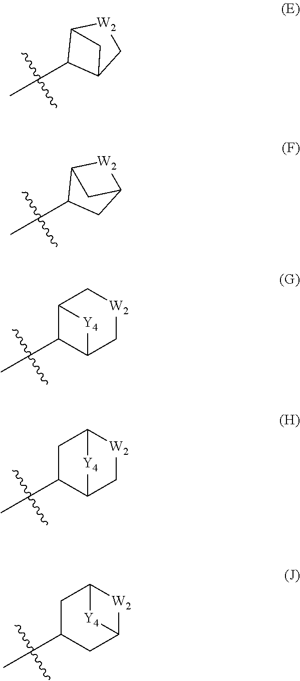

wherein Y₄ is selected from —CH₂—, —CH₂CH₂—,
and each of formulae (E), (F), (G), (H) and (J) is optionally substituted by one or more R^{a};
wherein R^{a} is as defined hereinbefore or in any one of paragraphs (106) to (114) hereinafter;
(iii) a group of the formula (K), (L) or (M) below:

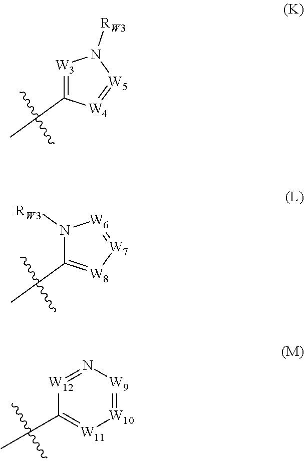

wherein:
R_{W3} is selected from hydrogen or (1-2C)alkyl;
W₃, W₄ and W₅ are selected from N or CH;
one of W₆ and W₇ is N and the other is CH and Wₐ is N or CH;
W₉, W₁₀, W₁₁ and W₁₂ are selected from N or CR_{w6} where R_{w6} is hydrogen, halo, (1-2C)alkyl or (1-2C)alkoxy; with the proviso that only one of W₉, W₁₀, W₁₁ and W₁₂ is N
(83) R¹¹ is —(CHR_{z})ₐ—Z¹¹,
   wherein R_{z} is hydrogen;
   wherein a is 0 or 1; and
   Z¹¹ is: —OR¹⁵, wherein R¹⁵ is (1-2C)alkyl; or
      —NHR¹⁷, wherein R¹⁷ is a group of the formula (A), (B), (C) or (D):

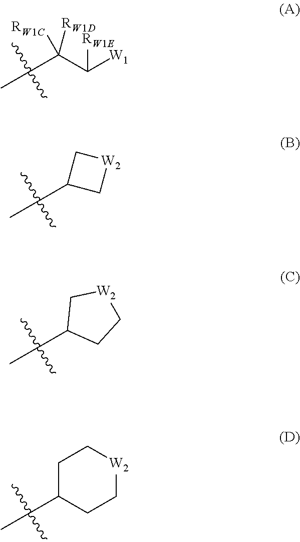

wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined hereinbefore or in any one of paragraphs (106) to (114) hereinafter; or
a group of the formula (K), (L) or (M) below:

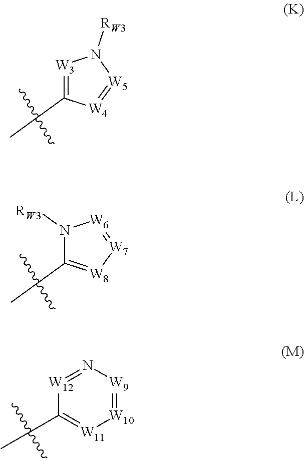

wherein:
R_{W3} is selected from hydrogen or methyl;
W₃, W₄ and W₅ are selected from N or CH;
one of W₆ and W₇ is N and the other is CH and Wₐ is N or CH;
W₉, W₁₀, W₁₁ and W₁₂ are selected from N or CR_{w6} where R_{w6} is hydrogen, halo, (1-2C)alkyl or (1-2C)alkoxy; with the proviso that only one of W₉, W₁₀, W₁₁ and W₁₂ is N
(84) R¹¹ is Z¹¹,
   Z¹¹ is: —OR¹⁵, wherein R¹⁵ is (1-2C)alkyl; or
      —NHR¹⁷, wherein R¹⁷ is selected from:
      a group of the formula (A), (B), (C), (D) or (M):

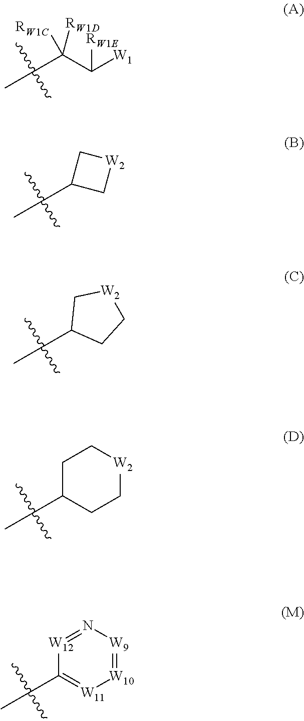

wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined hereinbefore or in any one of paragraphs (106) to (114) hereinafter;
W₉, W₁₀, W₁₁ and W₁₂ are selected from N or CR_{w6} where R_{w6} is hydrogen, halo, (1-2C)alkyl or (1-2C)alkoxy; with the proviso that only one of W₉, W₁₀, W₁₁ and W₁₂ is N;
(85) R¹¹ is Z¹¹,
   Z¹¹ is: —OR¹⁵, wherein R¹⁵ is methyl; or
      —NHR¹⁷, wherein R¹⁷ is selected from:
      a group of the formula (B), (C), (D) or (M):

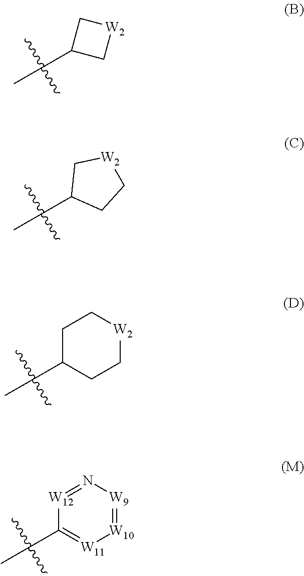

wherein
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by oxo (═O);
W₉, W₁₀, W₁₁ and W₁₂ are selected from N or CH; with the proviso that only one of W₉, W₁₀, W₁₁ and W₁₂ is N;
(86) R¹¹ is Z¹¹,
   Z¹¹ is: —OR¹⁵, wherein R¹⁵ is methyl; or
      —NHR¹⁷, wherein R¹⁷ is selected from:
      a group of the formula (B), (C) or (D):

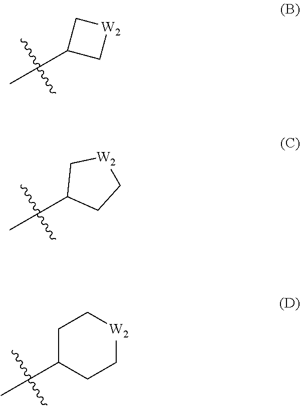

wherein
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by oxo (═O);
W₉, W₁₀, W₁₁ and W₁₂ are selected from N or CH; with the proviso that only one of W₉, W₁₀, W₁₁ and W₁₂ is N;
(87) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²,
         wherein Rᵥ is hydrogen or methyl;
         wherein d is 0 or 1; and
         Z¹² is —OR²⁰, R^{20a}, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rᵤ is hydrogen or methyl and e is 0, 1 or 2;
         R^{20a} is (1-4C)alkyl, (3-7C)cycloalkyl, phenyl, 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rᵤ is hydrogen or methyl and e is 0, 1 or 2;
         R²¹ and R²² are each independently selected from hydrogen, (1-6C)alkyl, (2-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[⁴ to 8-membered heterocyclyl] or —(CHR_{q})_{f}-[5 or 6 membered heteroaryl], wherein R_{q} is hydrogen or methyl and f is 0 or 1;
         or R²¹ and R²² are linked, such that, together with the nitrogen atom to which they are attached, they form a 4-8 membered heterocyclic ring;
         R²³ is hydrogen or (1-2C)alkyl;
         R²⁴ is (1-6C)alkyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
   wherein each of R²⁰, R^{20a}, R²¹, R²², R²³ or or any ring formed when R²¹ and R²² are linked, is optionally substituted with one or more R^{a};
   wherein R^{a} is as defined hereinbefore or in any one of paragraphs (106) to (114) hereinafter;
(88) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²
         wherein R_{y} is hydrogen or methyl;
         wherein d is 0 or 1; and
         Z¹² is —OR²⁰, R^{20a}, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rₐ is hydrogen and e is 0, 1 or 2;
         R^{20a} is (1-4C)alkyl, (4-6C)cycloalkyl, phenyl, 4 to 6-membered heterocyclyl, a 5 or 6 membered heteroaryl,
         R²¹ and R²² are each independently selected from hydrogen, (1-6C)alkyl, (3-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[4 to 8-membered heterocyclyl] or —(CHR_{q})_{f}-[5 or 6 membered heteroaryl], wherein R_{q} is hydrogen or methyl and f is 0 or 1;
         or R²¹ and R²² are linked, such that, together with the nitrogen atom to which they are attached, they form a 4-8 membered heterocyclic ring;
         R²³ is hydrogen or methyl;
         R²⁴ is (1-6C)alkyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
   wherein each of R²⁰, R^{20a}, R²¹, R²², R²³ or R²⁴, or any ring formed when R²¹ and R²² are linked, is optionally substituted with one or more R^{a};
   wherein R^{a} is as defined hereinbefore or in any one of paragraphs (106) to (114) hereinafter;
(89) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²
         wherein R_{y} is hydrogen;
         wherein d is 0 or 1; and
         Z¹² is —OR²⁰, R^{20a}, —NR²¹R²², —C(O)NR²¹R²² or —NR²³C(O)R²⁴;
         wherein R²⁰ is (1-4C)alkyl, (3-7C)cycloalkyl, phenyl, a carbon-linked 4 to 8-membered heterocyclyl, or a 5 or 6 membered heteroaryl;
         R^{20a} is (1-4C)alkyl, (4-6C)cycloalkyl, phenyl, 4 to 6-membered heterocyclyl, a 5 or 6 membered heteroaryl,
         R²¹ and R²² are each independently selected from hydrogen, (1-6C)alkyl, (3-7C)cycloalkyl, phenyl, a carbon-linked 4 to 8-membered heterocyclyl, or a 5 or 6 membered heteroaryl;
         or R²¹ and R²² are linked, such that, together with the nitrogen atom to which they are attached, they form a 4-8 membered heterocyclic ring;
         R²³ is hydrogen;
         R²⁴ is (1-6C)alkyl, (3-7C)cycloalkyl, phenyl, a carbon-linked 4 to 8-membered heterocyclyl, or a 5 or 6 membered heteroaryl;
   wherein each of R²⁰, R^{20a}, R²¹, R²² or or any ring formed when R²¹ and R²² are linked, is optionally substituted with one or more R^{a};
   wherein R^{a} is as defined hereinbefore or in any one of paragraphs (106) to (114) hereinafter;
(90) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²
         wherein R_{y} is hydrogen;
         wherein d is 0 or 1; and
         Z¹² is —OR²⁰, —NR²¹R²² or —C(O)NR²¹R²²;
      wherein R²⁰ is (1-4C)alkyl;
      R²¹ and R²² are each independently selected from hydrogen, (1-6C)alkyl, (3-7C)cycloalkyl, phenyl, a carbon-linked 4 to 8-membered heterocyclyl, or a 5 or 6 membered heteroaryl;
      or R²¹ and R²² are linked, such that, together with the nitrogen atom to which they are attached, they form a 4-8 membered heterocyclic ring;
   wherein each of R²⁰, R²¹ or R²², or any ring formed when R²¹ and R²² are linked, is optionally substituted with one or more R^{a};
   wherein R^{a} is as defined hereinbefore or in any one of paragraphs (106) to (114) hereinafter;
(91) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²
         wherein R_{y} is hydrogen;
         wherein d is 0 or 1; and
         Z¹² is —NR²¹R²²
         wherein R²¹ and R²² are each independently selected from hydrogen, (1-3C)alkyl, (4-6C)cycloalkyl, phenyl, a carbon-linked 4 to 6-membered heterocyclyl, or a 5 or 6 membered heteroaryl;
         or R²¹ and R²² are linked, such that, together with the nitrogen atom to which they are attached, they form a 4-8 membered heterocyclic ring;
   wherein each of R²¹ or R²², or any ring formed when R²¹ and R²² are linked, is optionally substituted with one or more R^{a};
   wherein R^{a} is as defined hereinbefore or in any one of paragraphs (106) to (114) hereinafter;
(92) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
      R¹² is —(CHRᵥ)_{d}—Z¹²
         wherein R_{y} is hydrogen;
         wherein d is 1; and
         Z¹² is —NR²¹R²², wherein R²¹ and R²² are each independently selected from
         hydrogen or methyl;
(93) R¹² is selected from one of the following options:
   (i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
   (ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy;
(94) R¹² is selected from halo, (1-2C)alkyl or (1-2C)alkoxy;
(95) R¹² is selected from chloro, fluoro, (1-2C)alkyl or (1-2C)alkoxy;
(96) R¹² is (1-2C)alkoxy;
(97) R¹³ is selected from cyano, halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein Rₒ is hydrogen or methyl;
      wherein h is 0 or 1; and
      Z¹² is —OR²⁵, R^{25a}, —NR²⁶R²⁷, —C(O)NR²⁶R²⁷ or —NR²⁸C(O)R²⁹;
         wherein R²⁵ is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0 or 1;
         R^{25a} is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ and i are as defined above;
         R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
         or R²⁶ and R²⁷ are linked, such that, together with the nitrogen atom to which they are attached, they form a 4-8 membered heterocyclic ring;
         R²⁸ is hydrogen or methyl;
         R²⁹ is (1-6C)alkyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
   wherein each of R²⁵, R^{25a}, R²⁶, R²⁷ or R²⁹ or any ring formed when R²⁶ and R²⁷ are linked, is optionally substituted with one or more R^{a};
   wherein R^{a} is as defined hereinbefore or in any one of paragraphs (106) to (114) hereinafter;
(98) R¹³ is selected from cyano, halo, (1-2C)alkyl, (1-2C)alkoxy, wherein any (1-2C)alkyl moiety is optionally substituted by one or more halo or (1-2C)alkoxy, or
   R¹³ is —(CHRₒ)ₕ—Z¹³,
      wherein Rₒ is hydrogen or methyl;
   wherein h is 0 or 1; and
   Z¹² is —OR²⁵, R^{25a}, —NR²⁶R²⁷, —C(O)NR²⁶R²⁷ or —NR²⁸C(O)R²⁹;
      wherein R²⁵ is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0 or 1;
      R^{25a} is (1-4C)alkyl, (4-6C)cycloalkyl, phenyl, a carbon-linked 4 to 8-membered heterocyclyl, a 5 or 6 membered heteroaryl, —(CHRₙ)ᵢ-(4-6C)cycloalkyl, —(CHRₙ)ᵢ-phenyl, —(CHRₙ)ᵢ-[4 to 6-membered heterocyclyl] or —(CHRₙ)ᵢ-[5 or 6 membered heteroaryl], wherein Rₙ and i are as defined above;
      R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
      or R²⁶ and R²⁷ are linked, such that, together with the nitrogen atom to which they are attached, they form a 4-8 membered heterocyclic ring;
      R²⁸ is hydrogen or methyl;
      R²¹ is (1-6C)alkyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
   wherein each of R²⁵, R^{25a}, R²⁶, R²⁷ or R²⁹ or any ring formed when R²⁶ and R²⁷ are linked, is optionally substituted with one or more R^{a};
   wherein R^{a} is as defined hereinbefore or in any one of paragraphs (106) to (114) hereinafter; or
(99) R¹³ is selected from halo, (1-2C)alkyl, (1-2C)alkoxy, wherein any (1-2C)alkyl moiety is optionally substituted by one or more halo or (1-2C)alkoxy, or
   R¹³ is —(CHR₀)ₕ—Z¹³,
      wherein Rₒ is hydrogen or methyl;
   wherein h is 0 or 1; and
   Z¹² is —OR²⁵, R²⁵, —NR²⁶R²⁷, or —C(O)NR²⁶R²⁷
   wherein R²⁵ is (1-4C)alkyl, a carbon-linked 4 to 8-membered heterocyclyl, or —(CHRₙ)ᵢ-[4 to 8-membered heterocyclyl], wherein Rₙ is hydrogen or methyl and i is 0 or 1;
   R^{25a} is (1-3C)alkyl, a 5 or 6 membered heteroaryl, —(CHRₙ)ᵢ-(4-6C)cycloalkyl, —(CHRₙ)ᵢ-phenyl, —(CHRₙ)ᵢ-[4 to 6-membered heterocyclyl] or —(CHRₙ)ᵢ-[5 or 6 membered heteroaryl], wherein Rₙ and i are as defined above;
   R²⁶ and R²⁷ are each independently selected from hydrogen, (1-6C)alkyl, a carbon-linked 4 to 8-membered heterocyclyl, or —(CHRₘ)ⱼ-[4 to 8-membered heterocyclyl], wherein Rₘ is hydrogen or methyl and j is 0 or 1;
   or R²⁶ and R²⁷ are linked, such that, together with the nitrogen atom to which they are attached, they form a 4-8 membered heterocyclic ring;
   wherein each of R²⁵, R^{25a}, R²⁶, R²⁷ or any ring formed when R²⁶ and R²⁷ are linked, is optionally substituted with one or more R^{a};
   wherein R^{a} is as defined hereinbefore or in any one of paragraphs (106) to (114) hereinafter;
(100) R¹³ is —(CHRₒ)ₕ—Z¹³,
   wherein Rₒ is hydrogen or methyl;
   wherein h is 0 or 1; and
   Z¹² is —OR²⁵, R²⁵ or —NR²⁶R²⁷
   wherein R²⁵ is (1-4C)alkyl;
   R^{25a} is (1-2C)alkyl, a 5 membered heteroaryl, —(CHRₙ)ᵢ-[6-membered heterocyclyl] or —(CHRₙ)ᵢ-[5 membered heteroaryl], wherein Rₙ and i are as defined above;
   R²⁶ and R²⁷ are each independently selected from hydrogen or (1-6C)alkyl;
   or R²⁶ and R²⁷ are linked, such that, together with the nitrogen atom to which they are attached, they form a 4-8 membered heterocyclic ring;
   wherein each of R²⁵, R²⁵, R²⁶, R²⁷ or any ring formed when R²⁶ and R²⁷ are linked, is optionally substituted with one or more R^{a};
   wherein R^{a} is as defined hereinbefore or in any one of paragraphs (106) to (114) hereinafter;
(101) R¹³ is —(CHRₒ)ₕ—Z¹³,
   wherein Rₒ is hydrogen;
   wherein h is 0 or 1; and
   Z¹² is —OR²⁵ or —NR²⁶R²⁷
   wherein R²⁵ is (1-2C)alkyl;
   R²⁶ and R²⁷ are each independently selected from hydrogen or (1-2C)alkyl;
   wherein each of R²⁵, R²⁶, R²⁷ or any ring formed when R²⁶ and R²⁷ are linked, is optionally substituted with one or more R^{a};
   wherein R^{a} is as defined hereinbefore or in any one of paragraphs (106) to (114) hereinafter;
(102) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen or methyl;
      wherein m is 0 or 1; and
      Z¹² is —OR³⁰—NR³¹R³², —C(O)NR³¹R³² or —NR³³C(O)R³⁴;
         wherein R³⁰ is (1-4C)alkyl, (3-7C)cycloalkyl, a carbon-linked 4 to 8-membered heterocyclyl, a 5 or 6 membered heteroaryl, —(CHRⱼ)ₒ-(3-7C)cycloalkyl, —(CHRⱼ)ₒ-[⁴ to 8-membered heterocyclyl] or —(CHRⱼ)ₒ-[⁵ or 6 membered heteroaryl], wherein R; is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 8-membered heterocyclyl, a 5 or 6 membered heteroaryl, —(CHRᵢ)ₚ-(3-7C)cycloalkyl, —(CHRᵢ)ₚ-[4 to 8-membered heterocyclyl] or —(CHRᵢ)ₚ-[5 or 6 membered heteroaryl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen or methyl;
         R³⁴ is (1-6C)alkyl, (3-7C)cycloalkyl, a carbon-linked 4 to 8-membered heterocyclyl, a 5 or 6 membered heteroaryl, —(CHRₕ)_{q}-(3-7C)cycloalkyl, —(CHRₕ)_{q}-[⁴ to 8-membered heterocyclyl] or —(CHRₕ)_{q}-[5 or 6 membered heteroaryl], wherein Rₕ is hydrogen or methyl and q is 0 or 1;
   wherein R³⁰, R³¹, R³² or R³⁴, or any ring formed when R³¹ and R³² are linked, is optionally substituted with one or more R^{a};
   wherein R^{a} is as defined hereinbefore or in any one of paragraphs (106) to (114) hereinafter;
(103) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen;
      wherein m is 0 or 1; and
      Z¹² is —OR³⁰, —NR³¹R³², —C(O)NR³¹R³² or —NR³³C(O)R³⁴;
         wherein R³⁰ is (1-4C)alkyl, (3-7C)cycloalkyl, a carbon-linked 4 to 8-membered heterocyclyl, —(CHRⱼ)ₒ-(3-7C)cycloalkyl, or —(CHRⱼ)ₒ-[4 to 8-membered heterocyclyl], wherein R; is hydrogen or methyl and o is 0 or 1;
         R³¹ and R³² are each independently selected from hydrogen, (1-6C)alkyl, (2-6C)alkanoyl, (3-7C)cycloalkyl, a carbon-linked 4 to 8-membered heterocyclyl, —(CHRᵢ)ₚ-(3-7C)cycloalkyl, or —(CHRᵢ)ₚ-[4 to 8-membered heterocyclyl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen or methyl;
         R³⁴ is (1-6C)alkyl, (3-7C)cycloalkyl, a carbon-linked 4 to 8-membered heterocyclyl, —(CHRₕ)_{q}-(3-7C)cycloalkyl, or —(CHRₕ)_{q}-[4 to 8-membered heterocyclyl], wherein Rₕ is hydrogen or methyl and q is 0 or 1;
   wherein R³⁰, R³¹, R³² or R³⁴, or any ring formed when R³¹ and R³² are linked, is optionally substituted with one or more R^{a};
   wherein R^{a} is as defined hereinbefore or in any one of paragraphs (106) to (114) hereinafter;
(104) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
   R¹⁴ is —(CHRₖ)ₘ—Z¹⁴,
      wherein Rₖ is hydrogen;
      wherein m is 0 or 1; and
      Z¹² is —OR³⁰, —NR³¹R³², —C(O)NR³¹R³² or —NR³³C(O)R³⁴;
         wherein R³⁰ is (1-4C)alkyl, a carbon-linked 4 to 8-membered heterocyclyl, or —(CHRⱼ)ₒ-[⁴ to 8-membered heterocyclyl], wherein R; is hydrogen or methyl and o is 0 or 1;
         R³¹ and R³² are each independently selected from hydrogen, (1-6C)alkyl, a carbon-linked 4 to 8-membered heterocyclyl, or —(CHRᵢ)ₚ-[4 to 8-membered heterocyclyl], wherein Rᵢ is hydrogen or methyl and p is 0 or 1
         or R³¹ and R³² are linked, such that, together with the nitrogen atom to which they are attached, they form a 4-8 membered heterocyclic ring;
         R³³ is hydrogen;
         R³⁴ is (1-6C)alkyl, a carbon-linked 4 to 8-membered heterocyclyl, or —(CHRₕ)_{q}-[⁴ to 8-membered heterocyclyl], wherein Rₕ is hydrogen or methyl and q is 0 or 1;
   wherein R³⁰, R³¹, R³² or R³⁴, or any ring formed when R³¹ and R³² are linked, is optionally substituted with one or more R^{a};
   wherein R^{a} is as defined hereinbefore or in any one of paragraphs (106) to (114) hereinafter;
(105) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy;
(106) each R^{a} is independently selected from the group consisting of oxo, halogen, cyano, hydroxy, (1-4C)alkyl, or a group

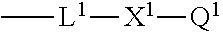

wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-4C)alkyl, or (3-6C)cycloalkyl;
(107) each R^{a} is independently selected from the group consisting of oxo, halogen, cyano, hydroxy, (1-2C)alkyl, or a group

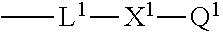

wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-2C)alkyl, or (3-6C)cycloalkyl;
(108) each R^{a} is independently selected from the group consisting of oxo, halogen, cyano, hydroxy, (1-2C)alkyl, or a group

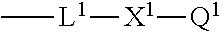

wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
   Q¹ is selected from the group consisting of hydrogen or (1-2C)alkyl;
(109) each R^{a} is independently selected from the group consisting of oxo, halogen, cyano, hydroxy, (1-2C)alkyl, or a group

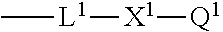

wherein:
   L¹ is absent;
   X¹ is absent or is selected from the group consisting of —O—, —C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
   Q¹ is selected from the group consisting of hydrogen or (1-2C)alkyl;
(110) each R^{a} is independently selected from the group consisting of oxo, halogen, cyano, hydroxy, (1-2C)alkyl, or a group

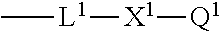

wherein:
   L¹ is absent;
   X¹ is absent or is selected from the group consisting of —O—, —C(O)—, —C(O)—N(R¹)—, or —NR¹⁴—, where R¹⁴ is hydrogen or (1-2C)alkyl;
   Q¹ is selected from the group consisting of hydrogen or (1-2C)alkyl;
   each R^{a} is independently selected from the group consisting of oxo, halogen, cyano, hydroxy, (1-4C)alkyl, or a group

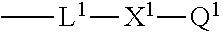

wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
   Q¹ is selected from the group consisting of hydrogen, (1-4C)alkyl, or (3-6C)cycloalkyl;
   wherein when Q¹ is (1-4C)alkyl or (3-6C)cycloalkyl, Q¹ is optionally substituted with one or more Q^{a}, where each Q^{a} is independently selected from the group consisting of (1-3C)alkoxy, (1-3C)haloalkyl, or (1-3C)haloalkoxy;
(111) each R^{a} is independently selected from the group consisting of oxo, halogen, cyano, hydroxy, (1-2C)alkyl, or a group

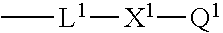

wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
Q¹ is selected from the group consisting of hydrogen, (1-3C)alkyl, or (3-6C)cycloalkyl;
wherein when Q¹ is (1-3C)alkyl or (3-6C)cycloalkyl, Q¹ is optionally substituted with one or more Q^{a}, where each Q^{a} is independently selected from the group consisting of (1-2C)alkoxy, (1-2C)haloalkyl, or (1-2C)haloalkoxy
(112) each R^{a} is independently selected from the group consisting of oxo, halogen, cyano, hydroxy, (1-2C)alkyl, or a group

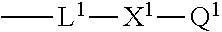

wherein:
   L¹ is absent or (1-2C)alkylene;
   X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
   Q¹ is selected from the group consisting of hydrogen or (1-3C)alkyl;
   wherein when Q¹ is (1-3C)alkyl, Q¹ is optionally substituted with one or more Q^{a}, where each Q^{a} is independently selected from the group consisting of (1-3C)alkoxy
(113) each R^{a} is independently selected from the group consisting of oxo, halogen, cyano, hydroxy, (1-2C)alkyl, or a group

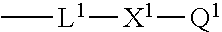

wherein:
L¹ is absent;
X¹ is absent or is selected from the group consisting of —O—, —C(O)—, —S(O)₀₂—, —C(O)—N(R¹⁴)—, —N(R¹⁴)—C(O)—, or —NR¹⁴—, where R¹⁴ is hydrogen or (1-2C)alkyl;
Q¹ is selected from the group consisting of hydrogen or (1-3C)alkyl;
wherein when Q¹ is (1-3C)alkyl, Q¹ is optionally substituted with one or more Q^{a}, where each Q^{a} is independently selected from the group consisting of (1-2C)alkoxy
(114) each R^{a} is independently selected from the group consisting of oxo, halogen, cyano, hydroxy, (1-2C)alkyl, or a group

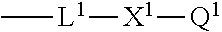

wherein:
L¹ is absent;
X¹ is absent or is selected from the group consisting of —O—, —C(O)—, —C(O)—N(R¹)—, or —NR¹⁴—, where R¹⁴ is hydrogen or (1-2C)alkyl;
Q¹ is selected from the group consisting of hydrogen or (1-3C)alkyl;
wherein when Q¹ is (1-3C)alkyl, Q¹ is substituted with one or more Q^{a}, where each Q^{a} is methoxy.

### Probe Compounds

(115) L is a (2-20C)alkylene linker, wherein the alkylene chain optionally further comprises one or more —O—, —C(O)—, —C(O)NR₁₀—, —NR₁₀C(O)—, —C(O)O—, —OC(O)—, piperazine or triazole ring linkages positioned either within the alkylene chain and/or at one of its termini, wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(116) L is a (3-20C)alkylene linker, wherein the alkylene chain optionally further comprises one or more —O—, —C(O)—, —C(O)NR₁₀—, —NR₁₀C(O)—, —C(O)O—, —OC(O)—, piperazine or triazole ring linkages positioned either within the alkylene chain and/or at one of its termini, wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(117) L is a (2-20C)alkylene linker, wherein the alkylene chain further comprises one to eight —O— linkages and/or one to four —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(118) L is a (3-20C)alkylene linker, wherein the alkylene chain further comprises one to eight —O— linkages and/or one to four —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(119) L is a (2-18C)alkylene linker, wherein the alkylene chain optionally further comprises one to five —O— linkages; and/or one to four —C(O)—, —C(O)NR₁₀—, —NR₁₀C(O)—, —C(O)O—, —OC(O)— linkages or one piperazine or triazole ring linkage positioned either within the alkylene chain and/or at one of its termini,
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(120) L is a (3-12C)alkylene linker, wherein the alkylene chain optionally further comprises one to five —O— linkages; and/or one to four —C(O)—, —C(O)NR₁₀—, —NR₁₀C(O)—, —C(O)O—, —OC(O)— or one piperazine or triazole ring linkages positioned either within the alkylene chain and/or at one of its termini,
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(121) L is a (2-18C)alkylene linker, wherein the alkylene chain further comprises one to five —O— linkages, and/or one to three —C(O)—, —C(O)NR₁₀— or —NR₁₀C(O)— linkages or one piperazine or triazole ring linkage positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(122) L is a (3-12C)alkylene linker, wherein the alkylene chain further comprises one to five —O— linkages and/or one to three —C(O)—, —C(O)NR₁₀— or —NR₁₀C(O)— linkages or one piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(123) L is a (2-18C)alkylene linker, wherein the alkylene chain further comprises one to five —O— linkages and/or one to three —C(O)—, —C(O)NR₁₀— or —NR₁₀C(O)— linkages or one piperazine or triazole ring linkage positioned either within the alkylene chain and/or at one of its termini and wherein R₁₀ is hydrogen or (1-2C)alkyl.
(124) L is a (3-12C)alkylene linker, wherein the alkylene chain further comprises one to five —O— linkages and/or one to three —C(O)—, —C(O)NR₁₀— or —NR₁₀C(O)— linkages or one piperazine or triazole ring linkage positioned either within the alkylene chain and/or at one of its termini and wherein R₁₀ is hydrogen or (1-2C)alkyl.
(125) L is a (2-18C)alkylene linker, wherein the alkylene chain further comprises one to three —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(126) L is a (3-12C)alkylene linker, wherein the alkylene chain further comprises one to three —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(127) L is a (2-18C)alkylene linker, wherein the alkylene chain further comprises one —O— linkage and/or one —C(O)—, —C(O)NR₁₀— or —NR₁₀C(O)— linkage or one piperazine or triazole ring linkage positioned either within the alkylene chain and/or at one of its termini, and wherein R₁₀ is hydrogen or (1-2C)alkyl.
(128) L is a (3-12C)alkylene linker, wherein the alkylene chain further comprises one —O— linkage and/or one —C(O)—, —C(O)NR₁₀— or —NR₁₀C(O)— linkage or one piperazine or triazole ring linkage positioned either within the alkylene chain and/or at one of its termini, and wherein R₁₀ is hydrogen or (1-2C)alkyl.
(129) L is a (2-17C)alkylene linker, wherein the alkylene chain optionally further comprises one to four-O— linkages and/or one to three —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(130) L is a (3-10C)alkylene linker, wherein the alkylene chain optionally further comprises one to four-O— linkages and/or one to three —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(131) L is a (2-17C)alkylene linker, wherein the alkylene chain further comprises one to four —O— linkages; and/or one to three —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(132) L is a (3-10C)alkylene linker, wherein the alkylene chain further comprises one to four —O— linkages; and/or one to three —C(O)—, —C(O)NR₁₀—, —NR₁₀C(O)—, —C(O)O—, —OC(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(133) L is a (2-17C)alkylene linker, wherein the alkylene chain further comprises one to four —O— linkages and/or one to three —C(O)—, —C(O)NR₁₀—, —NR₁₀C(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(134) L is a (3-10C)alkylene linker, wherein the alkylene chain further comprises one to four —O— linkages and/or one to three —C(O)—, —C(O)NR₁₀—, —NR₁₀C(O)—, piperazine or triazole ring linkages positioned either within the alkylene chain and/or at one of its termini;
   wherein R₁₀ is hydrogen or (1-2C)alkyl and wherein the alkylene chain is optionally substituted by one or more substituents independently selected from halo, hydroxy, cyano, amino, (1-2C)alkyl, (1-2C)hydroxyalkyl, (1-2C)haloalkyl or (1-2C)alkoxy.
(135) L is a (2-17C)alkylene linker, wherein the alkylene chain further comprises one to four —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(136) L is a (3-10C)alkylene linker, wherein the alkylene chain further comprises one to four —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(137) L is a (2-17C)alkylene linker, wherein the alkylene chain further comprises one to three —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(138) L is a (3-10C)alkylene linker, wherein the alkylene chain further comprises one to three —O— linkages and/or one to three —C(O)—, —C(O)NR₁₀— or —NR₁₀C(O)— linkages or one piperazine or triazole ring linkage positioned either within the alkylene chain and/or at one of its termini, and wherein R₁₀ is hydrogen or (1-2C)alkyl.
(139) L is a (2-17C)alkylene linker, wherein the alkylene chain further comprises one —O— linkage and/or one —C(O)—, —C(O)NR₁₀— or —NR₁₀C(O)— linkage or one triazole ring linkage positioned either within the alkylene chain and/or at one of its termini, and wherein R₁₀ is hydrogen or (1-2C)alkyl.
(140) L is a (3-10C)alkylene linker, wherein the alkylene chain further comprises one —O— linkage and/or one —C(O)—, —C(O)NR₁₀— or —NR₁₀C(O)— linkage or one piperazine or triazole ring linkage positioned either within the alkylene chain and/or at one of its termini, and wherein R₁₀ is hydrogen or (1-2C)alkyl.
(141) L is a linker having the formula:

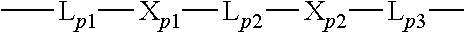

wherein
   Lₚ₁ is absent or a (1-8C)alkylene;
   Xₚ₁ is absent or selected from —O—, —C(O)—, —C(O)NRₚ₁—, —NRₚ₁C(O)—, —C(O)O—, —OC(O)— or a piperazine or triazole ring;
   Lₚ₂ is a (1-10C)alkylene or a group of the formula —[CH₂CH₂—O]ₐ—[CH₂]_{b}— or —[CH₂]_{b}—[O—CH₂CH₂]ₐ—, wherein a is 1 to 10 and b is 2 to 4;
   Xₚ₂ is absent or selected from —O—, —C(O)—, —C(O)NRₚ₁—, —NRₚ₁C(O)—, —C(O)O—, —OC(O)— or a piperazine or triazole ring;
   Lₚ₃ is absent or a (1-8C)alkylene;
   wherein Rₚ₁ is hydrogen or (1-2C)alkyl; and
   with the proviso that at least one of Xₚ₁ or Xₚ₂ is present;
(142) L is a linker having the formula:

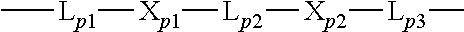

wherein
Lₚ₁ is absent or a (1-6C)alkylene;
Xₚ₁ is absent or selected from —O—, —C(O)—, —C(O)NRₚ₁—, —NRₚ₁C(O)—, —C(O)O—, —OC(O)— or piperazine or a triazole ring;
Lₚ₂ is a (1-10C)alkylene or a group of the formula —[CH₂CH₂O]ₐ—[CH₂]_{b}— or —[CH₂]_{b}—[O—CH₂CH₂]ₐ—, wherein a is 2 to 10 and b is 2 to 4;
Xₚ₂ is absent or selected from —O—, —C(O)—, —C(O)NRₚ₁—, —NRₚ₁C(O)—, —C(O)O—, —OC(O)— or a piperazine or triazole ring;
Lₚ₃ is absent or a (1-6C)alkylene;
wherein Rₚ₁ is hydrogen or methyl; and
with the proviso that at least one of Xₚ₁ or Xₚ₂ is present
(143) L is a linker having the formula:

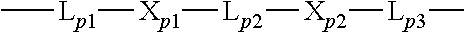

wherein
   Lₚ₁ is absent or a (2-6C)alkylene;
   Xₚ₁ is absent or selected from —O—, —C(O)—, —C(O)NRₚ₁—, —NRₚ₁C(O)— or a piperazine or triazole ring;
   Lₚ₂ is a (2-10C)alkylene or a group of the formula —[CH₂CH₂O]ₐ—[CH₂]_{b}— or —[CH₂]_{b}—[O—CH₂CH₂]ₐ—, wherein a is 2 to 10 and b is 2 to 4;
   Xₚ₂ is absent or selected from —O—, —C(O)—, —C(O)NRₚ₁—, —NRₚ₁C(O)— or a piperazine or triazole ring;
   Lₚ₃ is absent or a (1-6C)alkylene;
   wherein Rₚ₁ is hydrogen or methyl; and
   with the proviso that at least one of Xₚ₁ or Xₚ₂ is present;
(144) L is a linker having the formula:

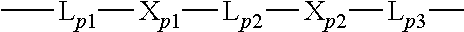

wherein
Lₚ₁ is absent or a (2-6C)alkylene;
Xₚ₁ is absent or selected from —O—, —C(O)—, —C(O)NRₚ₁—, —NRₚ₁C(O)— or a piperazine or triazole ring;
Lₚ₂ is a (2-6C)alkylene or a group of the formula —[CH₂CH₂—O]ₐ—[CH₂]_{b}— or —[CH₂]_{b}—[O—CH₂CH₂]ₐ—, wherein a is 2 to 10 and b is 2 to 4;
Xₚ₂ is absent or selected from —O—, —C(O)—, —C(O)NRₚ₁—, —NRₚ₁C(O)— or a piperazine or triazole ring;
Lₚ₃ is absent or a (2-6C)alkylene;
wherein Rₚ₁ is hydrogen or methyl; and
with the proviso that at least one of Xₚ₁ or Xₚ₁ is present;
(145) L is a linker having the formula:

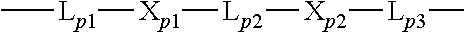

wherein
   Lₚ₁ is absent;
   Xₚ₁ is absent;
   Lₚ₂ is absent or a (2-6C)alkylene or a group of the formula —[CH₂CH₂-]ₐ-[CH₂]_{b}— or —[CH₂]_{b}—[O—CH₂CH₂]ₐ—, wherein a is 2 to 10 and b is 2 to 4;
   Xₚ₂ is selected from —O—, —C(O)—, —C(O)NRₚ₁—, —NRₚ₁C(O)— or a triazole ring;
   Lₚ₃ is absent or a (2-6C)alkylene;
   wherein Rₚ₁ is hydrogen or methyl; and
with the proviso that at least one of Xₚ₁ or Xₚ₂ is present;
(146) L is a linker having the formula:

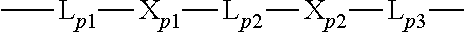

wherein
   Lₚ₁ is a (2-6C)alkylene;
   Xₚ₁ is selected from —O—, —C(O)NRₚ₁—, —NRₚ₁C(O)— or a piperazine or triazole ring;
   Lₚ₁ is a (2-6C)alkylene or a group of the formula —[CH₂CH₂—O]ₐ—[CH₂]_{b}— or —[CH₂]_{b}—[O—CH₂CH₂]ₐ—, wherein a is 2 to 10 and b is 2 to 4;
   Xₚ₂ is selected from —O—, —C(O)NRₚ₁—, —NRₚ₁C(O)— or a triazole ring;
   Lₚ₃ is a (2-6C)alkylene;
   wherein Rₚ₁ is hydrogen or methyl; and
   with the proviso that at least one of Xₚ₁ or Xₚ₂ is present;
(147) Q is a detection moiety selected from the group consisting of a fluorophore, an oligonucleotide, a biomolecule, a molecular sensor, a protein, or a peptide;
(148) Q is a fluorophore;
(149) Q is a fluorophore selected from the group consisting of AlexaFluor dyes, Cyanine dyes, fluorescein, BODIPY or BODIPY derivatives (e.g. BODIPY TMR), TAMRA, Oregon Green dyes, FITC, Ru(bpy)3, Rhodamine dyes, Acridine orange, and Texas Red;
(150) Q is a fluorophore selected from the group consisting of AlexaFluor-647, AlexaFluor-633, AlexaFluor-594, AlexaFluor-488, Cyanine-5B, Cyanine-3B, Fluorescein, BODIPY or BODIPY derivatives (e.g. BODIPY TMR), TAMRA, Oregon Green 488, Oregon Green 514, FITC, Ru(bpy)3, Rhodamine dyes, Acridine orange, and Texas Red;
(151) X is a functional group that is capable of reacting with a functional group present on a detection moiety so as to covalently bind the detection moiety to the compound of formula (I);
(152) X is a functional group that is capable of reacting with a functional group present on a detection moiety so as to covalently bind the detection moiety to the compound of formula (I), wherein the detection moiety selected from the group consisting of a fluorophore, an oligonucleotide, a biomolecule, a molecular sensor, a protein, or a peptide;
(153) X is a functional group X that is capable of reacting with a functional group present on a fluorophore so as to covalently bind the fluorophore to the compound of formula (I);
(154) X is a functional group that is capable of reacting with a functional group present on a fluorophore so as to covalently bind the fluorophore to the compound of formula (I), and wherein the fluorophore is as defined in paragraph (149) or (150) above;
(155) X is a functional group selected from halo, N₃, or ethynyl (—C≡CH);
(156) X is ethynyl (—C≡CH) or N₃;
(157) Lₓ is a linker group L as defined in any one of paragraphs (115) to (146) above
(158) Lₓ is a (1-8C)alkylene linker, wherein the alkylene chain optionally further comprises one or more —O—, —C(O)NR₁₀—, —NR₁₀C(O)—, —C(O)O—, —OC(O)— or triazole ring linkages positioned either within the alkylene chain and/or at one of its termini, wherein R₁₀ is hydrogen or (1-2C)alkyl;
(159) Lₓ is a (1-8C)alkylene linker, wherein the alkylene chain optionally further comprises one —O—, —C(O)NR₁₀—, —NR₁₀C(O)—, —C(O)O—, —OC(O)— or triazole ring linkage positioned either within the alkylene chain and/or at one of its termini, wherein R₁₀ is hydrogen or (1-2C)alkyl;
(160) Lₓ is a (1-5C)alkylene linker; or
(161) Lₓ is a (1-3C)alkylene linker.

Suitably, R² is as defined in numbered paragraph (1) or (2) above. Most suitably, R² is as defined in numbered paragraph (1) above.

Suitably, R⁴ is as defined in any one of numbered paragraphs (4) to (7) above. More suitably, R⁴ is as defined in any one of numbered paragraphs (5) to (7) above. Most suitably, R⁴ is as defined in numbered paragraph (6) above.

Suitably, R³ is as defined in any one of numbered paragraphs (10) to (14) above. More suitably, R³ is as defined in any one of numbered paragraphs (12) to (14) above. Most suitably, R³ is as defined in numbered paragraph (14) above.

Suitably, R³ and R⁴ are as defined in any one of numbered paragraphs (8) to (21) above. More suitably, R³ and R⁴ are as defined in any one of numbered paragraphs (12) to (21) above. Even more suitably, R³ and R⁴ are as defined in any one of numbered paragraphs (16) to (21) above. Yet even more suitably, R³ and R⁴ are as defined in any one of numbered paragraphs (18) to (21) above. Most suitably, R³ and R⁴ are as defined in numbered paragraph (21) above.

Suitably, R⁵ is as defined in any one of numbered paragraphs (22) to (24) above. More suitably, R⁵ is as defined in any one of numbered paragraphs (23) or (24) above. Most suitably, R⁵ is as defined numbered paragraph (24) above.

Suitably, R⁶ is as defined in any one of numbered paragraphs (28) to (36) above. More suitably, R⁶ is as defined in any one of numbered paragraphs (32) to (36) above. Even more suitably, R⁶ is as defined in any one of numbered paragraphs (34) to (36) above. Most suitably, R⁶ is as defined numbered paragraph (36) above.

Suitably, Y₁ is as defined in numbered paragraphs (37), (37a) or (38) above. Most suitably, Y₁ is as defined in numbered paragraph (37) above.

Suitably, Y₂ is as defined in any one of numbered paragraphs (39) to (44) above. More suitably, Y₂ is as defined in any one of numbered paragraphs (41) to (44) above. Even more suitably, Y₂ is as defined in numbered paragraphs (42), (43) or (44) above. Most suitably, Y₂ is as defined in numbered paragraph (43) above.

Suitably, A₁ is as defined in any one of numbered paragraphs (45) to (47) above. Most suitably, A₁ is as defined in numbered paragraph (47) above.

Suitably, A₂ is as defined in any one of numbered paragraphs (48) to (51) above. Most suitably, A₂ is as defined in numbered paragraph (48) above.

Suitably, A₃ is as defined in any one of numbered paragraphs (52) to (55) above. Most suitably, A₃ is as defined in numbered paragraph (52) above.

Suitably, A₄ is as defined in any one of numbered paragraphs (56) to (59) above. Most suitably, A₄ is as defined in numbered paragraphs (58) above.

Suitably, A₁, A₂, A₃ and A₄ are as defined in any one of numbered paragraphs (60) to (67) above. More suitably, A₁, A₂, A₃ and A₄ are as defined in any one of numbered paragraphs (63) to (67) above. Even more suitably, A₁, A₂, A₃ and A₄ are as defined in any one of numbered paragraphs (64) to (67) above. Most suitably, A₁, A₂, A₃ and A₄ are as defined in numbered paragraph (64) above.

Suitably, R¹¹ is as defined in any one of numbered paragraphs (74) to (86) above. More suitably, R¹¹ is as defined in any one of numbered paragraphs (80) to (86) above. Even more suitably, R¹¹ is as defined in any one of numbered paragraphs (84) to (86) above. Most suitably, R¹¹ is as defined numbered paragraph (85) or (86) above.

Suitably, R¹² is as defined in any one of numbered paragraphs (89) to (96) above. More suitably, R¹² is as defined in any one of numbered paragraphs (91) to (96) above. Most suitably, R¹² is as defined numbered paragraphs (94), (95) or (96) above.

Suitably, R¹³ is as defined in any one of numbered paragraphs (98) to (101) above. More suitably, R¹³ is as defined in numbered paragraph (99) to (101) above. Most suitably, R¹³ is as defined numbered paragraph (101) above.

Suitably, R¹⁴ is as defined in any one of numbered paragraphs (102) to (105) above. More suitably, R¹⁴ is as defined in numbered paragraph (103), (104) or (105) above. Most suitably, R¹⁴ is as defined numbered paragraph (105) above.

Suitably, R^{a} is as defined in any one of numbered paragraphs (106) to (114) above. More suitably, R^{a} is as defined in any one of numbered paragraphs (109) to (114) above. Even more suitably, R^{a} is as defined in any one of numbered paragraphs (111) to (114) above. Most suitably, R^{a} is as defined numbered paragraphs (112), (113) or (114) above.

Suitably, L is as defined in any one of numbered paragraphs (115) to (146) above. More suitably, L is as defined in any one of numbered paragraphs (129) to (146) above. Even more suitably, L is as defined in any one of numbered paragraphs (139) to (146) above. Most suitably, L is as defined numbered paragraphs (144), (145) or (146) above.

Suitably, Q is as defined in any one of numbered paragraphs (147) to (150) above. More suitably, Q is as defined in any one of numbered paragraphs (148) to (150) above. Most suitably, Q is as defined numbered paragraph (149) or (150) above.

Suitably, X is as defined in any one of numbered paragraphs (151) to (156) above. More suitably, X is as defined in any one of numbered paragraphs (153) to (156) above. More suitably, X is as defined in any one of numbered paragraphs (154) to (156) above. Most suitably, X is as defined numbered paragraph (156) above.

Suitably, Lₓ is as defined in any one of numbered paragraphs (157) to (161) above. More suitably, Lₓ is as defined in any one of numbered paragraphs (159) to (161) above. Most suitably, Lₓ is as defined numbered paragraph (161) above.

In a particular group of compounds of the invention, compounds have a structure according to sub-formulae (I-I) to (I-XLV), or a pharmaceutically acceptable salt, hydrate and/or solvate thereof, as shown herein. It will be understood that in the following embodiments, definitions of R², R³, R⁴, R⁵, Y₁, Y₂, A₁, A₂, A₃, A₄, L, Lₓ, X, Q and any associated substituent groups are only applicable when present in sub-formulae (I-I) to (I-XLV).

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (3) above; R³ is as defined in numbered paragraph (8) above; R⁵ is as defined in numbered paragraph (22) above; Y₁ is as defined in numbered paragraph (37) above; Y₂ is as defined in numbered paragraph (39) above; A₁, A₂, A₃ and A₄ are as defined in numbered paragraph (60) above; R¹¹ is as defined in numbered paragraph (68) above; R¹² is as defined in numbered paragraph (87) above; R¹³ is as defined in numbered paragraph (97) above; R¹⁴ is as defined in numbered paragraph (102) above; R^{a} is as defined in numbered paragraph (106) above; L is as defined in numbered paragraph (115) above; Q is as defined in numbered paragraphs (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (6) above; R³ is as defined in numbered paragraph (8) above; R⁵ is as defined in numbered paragraph (22) above; Y₁ is as defined in numbered paragraph (37) above; Y₂ is as defined in numbered paragraph (39) above; A₁, A₂, A₃ and A₄ are as defined in numbered paragraph (61) above; R¹¹ is as defined in numbered paragraph (68) above; R¹² is as defined in numbered paragraph (87) above; R¹³ is as defined in numbered paragraph (97) above; R¹⁴ is as defined in numbered paragraph (102) above; R^{a} is as defined in numbered paragraph (106) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (3) above; R³ is as defined in numbered paragraph (14) above; R⁵ is as defined in numbered paragraph (22) above; Y₁ is as defined in numbered paragraph (37) above; Y₂ is as defined in numbered paragraph (39) above; A₁, A₂, A₃ and A₄ are as defined in numbered paragraph (61) above; R¹¹ is as defined in numbered paragraph (68) above; R¹² is as defined in numbered paragraph (87) above; R¹³ is as defined in numbered paragraph (97) above; R¹⁴ is as defined in numbered paragraph (102) above; R^{a} is as defined in numbered paragraph (106) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R³ and R⁴ are as defined in numbered paragraph (18) above; R⁵ is as defined in numbered paragraph (22) above; Y₁ is as defined in numbered paragraph (37) above; Y₂ is as defined in numbered paragraph (39) above; A₁, A₂, A₃ and A₄ are as defined in numbered paragraph (61) above; R¹¹ is as defined in numbered paragraph (68) above; R¹² is as defined in numbered paragraph (87) above; R¹³ is as defined in numbered paragraph (97) above; R¹⁴ is as defined in numbered paragraph (102) above; R^{a} is as defined in numbered paragraphs (106) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraphs (1) above; R⁴ is as defined in numbered paragraph (3) above; R³ is as defined in numbered paragraph (8) above; R⁵ is as defined in numbered paragraph (23) above; R⁶ is as defined in numbered paragraph (34) above; Y₁ is as defined in numbered paragraph (37) above; Y₂ is as defined in numbered paragraph (39) above; A₁, A₂, A₃ and A₄ are as defined in numbered paragraph (61) above; R¹¹ is as defined in numbered paragraph (68) above; R¹² is as defined in numbered paragraph (87) above; R¹³ is as defined in numbered paragraph (97) above; R¹⁴ is as defined in numbered paragraph (102) above; R^{a} is as defined in numbered paragraph (106) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (3) above; R³ is as defined in numbered paragraph (8) above; R⁵ is as defined in numbered paragraph (22) above; Y₁ is as defined in numbered paragraph (37) above; Y₂ is as defined in numbered paragraph (43) above; A₁, A₂, A₃ and A₄ are as defined in numbered paragraph (64) above; R¹¹ is as defined in numbered paragraph (68) above; R¹² is as defined in numbered paragraph (87) above; R¹³ is as defined in numbered paragraph (97) above; R¹⁴ is as defined in numbered paragraph (102) above; R^{a} is as defined in numbered paragraph (106) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraphs (3) above; R³ is as defined in numbered paragraph (8) above; R⁵ is as defined in numbered paragraph (22) above; Y₁ is as defined in numbered paragraph (37) above; Y₂ is as defined in numbered paragraph (39) above; A₁, A₂, A₃ and A₄ are as defined in numbered paragraph (61) above; R¹¹ is as defined in numbered paragraph (84) above; R¹² is as defined in numbered paragraph (88) above; R¹³ is as defined in numbered paragraph (97) above; R¹⁴ is as defined in numbered paragraph (102) above; R^{a} is as defined in numbered paragraph (106) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (3) above; R³ is as defined in numbered paragraph (8) above; R⁵ is as defined in numbered paragraph (22) above; Y₁ is as defined in numbered paragraph (37) above; Y₂ is as defined in numbered paragraph (39) above; A₁, A₂, A₃ and A₄ are as defined in numbered paragraph (61) above; R¹¹ is as defined in numbered paragraph (86) above; R¹² is as defined in numbered paragraph (87) above; R¹³ is as defined in numbered paragraph (97) above; R¹⁴ is as defined in numbered paragraph (102) above; R^{a} is as defined in numbered paragraph (106) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (3) above; R³ is as defined in numbered paragraph (8) above; R⁵ is as defined in numbered paragraph (22) above; Y₁ is as defined in numbered paragraph (37) above; Y₂ is as defined in numbered paragraph (39); A₁, A₂, A₃ and A₄ are as defined in numbered paragraph (61) above; R¹¹ is as defined in numbered paragraph (68) above; R¹² is as defined in numbered paragraph (87) above; R¹³ is as defined in numbered paragraph (101) above; R¹⁴ is as defined in numbered paragraph (102) above; R^{a} is as defined in numbered paragraph (106) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (3) above; R³ is as defined in numbered paragraph (8) above; R⁵ is as defined in numbered paragraph (22) above; Y₁ is as defined in numbered paragraph (37) above; Y₂ is as defined in numbered paragraph (39) above; A₁, A₂, A₃ and A₄ are as defined in numbered paragraph (61) above; R¹¹ is as defined in numbered paragraph (68) above; R¹² is as defined in numbered paragraph (87) above; R¹³ is as defined in numbered paragraph (97) above; R¹⁴ is as defined in numbered paragraphs (102); R^{a} is as defined in numbered paragraph (113) above; L is as defined in numbered paragraph (115) above; Q is as defined in numbered paragraph (147) above; X is as defined in numbered paragraph (151); and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (4) above; R³ is as defined in numbered paragraph (10) above; R⁵ is as defined in numbered paragraph (22) above; Y₁ is as defined in numbered paragraph (37) above; Y₂ is as defined in numbered paragraph (39) above; A₁, A₂, A₃ and A₄ are as defined in numbered paragraph (63) above; R¹¹ is as defined in numbered paragraph (74) above; R¹² is as defined in numbered paragraph (89) above; R¹³ is as defined in numbered paragraph (98) above; R¹⁴ is as defined in numbered paragraph (102) above; R^{a} is as defined in numbered paragraph (109) above; L is as defined in numbered paragraph (115) above; Q is as defined in numbered paragraph (147) above; X is as defined in numbered paragraph (151) above; and Lₓ is as defined in numbered paragraph (157) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (5) above; R³ is as defined in numbered paragraph (12) above; R⁵ is as defined in numbered paragraph (23) above; Y₁ is as defined in numbered paragraph (37) above; Y₂ is as defined in numbered paragraph (41) above; A₁, A₂, A₃ and A₄ are as defined in numbered paragraph (64) above; R¹¹ is as defined in numbered paragraphs (80) above; R¹² is as defined in numbered paragraph (92) above; R¹³ is as defined in numbered paragraph (99) above; R¹⁴ is as defined in numbered paragraph (103) above; R^{a} is as defined in numbered paragraph (111) above; L is as defined in numbered paragraph (129) to above; Q is as defined in numbered paragraph (148) above; X is as defined in numbered paragraph (153) above; and Lₓ is as defined in numbered paragraph (159) above.

In an embodiment of the compounds of sub-formulae (I-I) to (I-XLV), or a pharmaceutically acceptable salt, hydrate and/or solvate thereof: R² is as defined in numbered paragraph (1) above; R⁴ is as defined in numbered paragraph (5) above; R³ is as defined in numbered paragraph (12) above; R⁵ is as defined in numbered paragraph (23) above; Y₁ is as defined in numbered paragraph (37) above; Y₂ is as defined in numbered paragraph (41) above; A₁, A₂, A₃ and A₄ are as defined in numbered paragraph (64) above; R¹¹ is as defined in numbered paragraph (84) above; R¹² is as defined in numbered paragraph (94) above; R¹³ is as defined in numbered paragraph (101) above; R¹⁴ is as defined in numbered paragraph (105) above; R^{a} is as defined in numbered paragraph (112) above; L is as defined in numbered paragraph (139) above; Q is as defined in numbered paragraph (149) above; X is as defined in numbered paragraph (156) above; and Lₓ is as defined in numbered paragraph (161) above.

In an embodiment of the compounds of sub-formulae (I-I) to (I-XLV), or a pharmaceutically acceptable salt, hydrate and/or solvate thereof: R² is as defined in numbered paragraph (1) above; R³ and R⁴ are as defined in numbered paragraph (12) above; R⁵ is as defined in numbered paragraph (22) above; R⁶ is as defined in numbered paragraph (25) above; Y₁ and Y₂ are as defined in numbered paragraph (42) above; A₁, A₂, A₃ and A₄ are as defined in any one of numbered paragraphs (64) to (67) above; R¹¹ is as defined in numbered paragraph (70) above; R¹² is as defined in numbered paragraph (90) above; R¹³ is as defined in numbered paragraph (99) above; R¹⁴ is as defined in numbered paragraph (105) above; R^{a} is as defined in any one of numbered paragraphs (112) to (114) above; L is as defined in numbered paragraph (139) above; Q is as defined in numbered paragraph (149) above; X is as defined in numbered paragraph (154) above; and Lₓ is as defined in numbered paragraph (161) above.

In an embodiment of the compounds of sub-formulae (I-I) to (I-XLV), or a pharmaceutically acceptable salt, hydrate and/or solvate thereof: R² is as defined in numbered paragraph (1) above; R³ and R⁴ are as defined in numbered paragraph (15) above; R⁵ is as defined in numbered paragraph (22) above; R⁶ is as defined in numbered paragraph (29) above; Y₁ and Y₂ are as defined in numbered paragraph (42) above; A₁ is as defined in numbered paragraph (45) above; A₂ is as defined in numbered paragraph (48) above; A₃ is as defined in numbered paragraph (52) above; A₄ is CH; R¹¹ is as defined in numbered paragraph (80) above; R¹² is as defined in numbered paragraph (95) above; R¹³ is as defined in numbered paragraph (100) above; and R^{a} is as defined in any one of numbered paragraphs (112) to (114) above.

In an embodiment of the compounds of sub-formulae (I-I) to (I-XLV), or a pharmaceutically acceptable salt, hydrate and/or solvate thereof: R² is as defined in numbered paragraph (1) above; R³ and R⁴ are as defined in numbered paragraph (15) above; R⁵ is as defined in numbered paragraph (22) above; R⁶ is as defined in numbered paragraph (36) above; Y₁ and Y₂ are as defined in numbered paragraph (42) above; A₁ is as defined in numbered paragraph (45) above; A₂ is as defined in numbered paragraph (48) above; A₃ is as defined in numbered paragraph (52) above; A₄ is CH; R¹¹ is as defined in numbered paragraph (84) above; R¹² is as defined in numbered paragraph (95) above; R¹³ is as defined in numbered paragraph (101) above; and R^{a} is as defined in any one of numbered paragraphs (112) to (114) above.

In an embodiment of the compounds of sub-formulae (I-I) to (I-XLV), or a pharmaceutically acceptable salt, hydrate and/or solvate thereof: R² is as defined in numbered paragraph (1) above; R³ and R⁴ are as defined in numbered paragraph (15) above; R⁵ is as defined in numbered paragraph (22) above; R⁶ is as defined in numbered paragraph (36) above; Y₁ and Y₂ are as defined in numbered paragraph (42) above; A₁ is as defined in numbered paragraph (45) above; A₂ is as defined in numbered paragraph (48) above; A₃ is as defined in numbered paragraph (52) above; A₄ is CH; R¹¹ is as defined in numbered paragraph (86) above; R¹² is as defined in numbered paragraph (95) above; R¹³ is as defined in numbered paragraph (101) above; R^{a} is as defined in any one of numbered paragraphs (112) to (114) above.

In an aspect of the invention, the compounds of formula I or sub-formulae (I-I) to (I-XLV), or a pharmaceutically acceptable salt, hydrate and/or solvate thereof, R², R³, R⁴, R⁵, Y₁, Y₂, A₁, A₂, A₃, A₄, L, Lₓ, X, Q and any associated substituent groups are as defined above, with the proviso that one of R¹² or R¹³ is not a group:

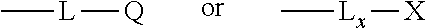

wherein L is a linker;
Lₓ is a linker;
X is a functional group; and
Q is a detection moiety.

In an embodiment of the compounds of sub-formulae (I-I) to (I-XLV), or a pharmaceutically acceptable salt, hydrate and/or solvate thereof, R², R³, R⁴, R⁵, Y₁, Y₂, A₁, A₂, A₃, A₄, L, Lₓ, X, Q and any associated substituent groups are as defined above, with the proviso that one of R¹² or R¹³ is a group:

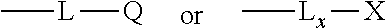

wherein L is a linker;
Lₓ is a linker;
X is a functional group; and
Q is a detection moiety.

In an embodiment of the compounds of sub-formulae (I-I) to (I-XLV), or a pharmaceutically acceptable salt, hydrate and/or solvate thereof, R², R³, R⁴, R⁵, Y₁, Y₂, A₁, A₂, A₃, A₄, L, Lₓ, X, Q and any associated substituent groups are as defined above, with the proviso that one of R¹² or R¹³ is a group:

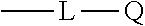

wherein L is a linker as defined in any one of numbered paragraphs (139) to (148) above; and
Q is a detection moiety as defined in any one of numbered paragraphs (151) to (156) above.

In an embodiment of the compounds of sub-formulae (I-I) to (I-XLV), or a pharmaceutically acceptable salt, hydrate and/or solvate thereof, R², R³, R⁴, R⁵, Y₁, Y₂, A₁, A₂, A₃, A₄, L, Lₓ, X, Q and any associated substituent groups are as defined above, with the proviso that one of R¹² or R¹³ is a group:

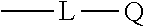

wherein L is a linker as defined in any one of numbered paragraphs (144), (145) or (146) above; and
Q is a detection moiety as defined in numbered paragraph (149) or (150) above.

In a particular group of compounds of Formula (I), or sub-formulae (I-I) to (I-XLV) defined herein, R² is as defined in paragraph (1) above.

In a particular group of compounds of Formula (I), or sub-formulae (I-I) to (I-XLV) defined herein, R⁴ is as defined in numbered paragraph (5) above.

In a particular group of compounds of Formula (I), or sub-formulae (I-I) to (I-XLV) defined herein, R⁴ is as defined in numbered paragraph (6) above.

In a particular group of compounds of Formula (I), or sub-formulae (I-I) to (I-XLV) defined herein, R⁴ is as defined in numbered paragraph (7) above.

In a particular group of compounds of Formula (I), or sub-formulae (I-I) to (I-XLV) defined herein, R³ is as defined in numbered paragraph (12) above.

In a particular group of compounds of Formula (I), or sub-formulae (I-I) to (I-XLV) defined herein, R³ is as defined in numbered paragraph (13) above.

In a particular group of compounds of Formula (I), or sub-formulae (I-I) to (I-XLV) defined herein, R³ is as defined in numbered paragraph (14) above.

In a particular group of compounds of Formula (I), or sub-formulae (I-I) to (I-XLV) defined herein, R³ and R⁴ are as defined in numbered paragraph (18) above.

In a particular group of compounds of Formula (I), or sub-formulae (I-I) to (I-XLV) defined herein, R³ and R⁴ are as defined in numbered paragraph (19) above.

In a particular group of compounds of Formula (I), or sub-formulae (I-I) to (I-XLV) defined herein, R³ and R⁴ are as defined in numbered paragraph (20) above.

In a particular group of compounds of Formula (I), or sub-formulae (I-I) to (I-XLV) defined herein, R³ and R⁴ are as defined in numbered paragraph (21) above.

In a particular group of compounds of Formula (I), or sub-formulae (I-I) to (I-XLV) defined herein, R⁵ is as defined in numbered paragraph (23) above.

In a particular group of compounds of Formula (I), or sub-formulae (I-I) to (I-XLV) defined herein, R⁵ is as defined in numbered paragraph (24) above.

In a particular group of compounds of Formula (I), or sub-formulae (I-I) to (I-XLV) defined herein, R⁶ is as defined in numbered paragraph (34) above.

In a particular group of compounds of Formula (I), or sub-formulae (I-I) to (I-XLV) defined herein, R⁶ is as defined in numbered paragraph (35) above.

In a particular group of compounds of Formula (I), or sub-formulae (I-I) to (I-XLV) defined herein, R⁶ is as defined in numbered paragraph (36) above.

In a particular group of compounds of Formula (I), or sub-formulae (I-I) to (I-XLV) defined herein, A₁, A₂, A₃ and A₄ are as defined in numbered paragraph (64) above.

In a particular group of compounds of Formula (I), or sub-formulae (I-I) to (I-XLV) defined herein, R¹¹ is as defined in numbered paragraph (84) above.

In a particular group of compounds of Formula (I), or sub-formulae (I-I) to (I-XLV) defined herein, R¹¹ is as defined in numbered paragraph (85) above.

In a particular group of compounds of Formula (I), or sub-formulae (I-I) to (I-XLV) defined herein, R¹¹ is as defined in numbered paragraph (86) above.

In a particular group of compounds of Formula (I), or sub-formulae (I-I) to (I-XLV) defined herein, R¹² is as defined in numbered paragraph (94) above.

In a particular group of compounds of Formula (I), or sub-formulae (I-I) to (I-XLV) defined herein, R¹² is as defined in numbered paragraph (95) above.

In a particular group of compounds of Formula (I), or sub-formulae (I-I) to (I-XLV) defined herein, R¹² is as defined in numbered paragraph (96) above.

In a particular group of compounds of Formula (I), or sub-formulae (I-I) to (I-XLV) defined herein, R¹³ is as defined in numbered paragraph (99) above.

In a particular group of compounds of Formula (I), or sub-formulae (I-I) to (I-XLV) defined herein, R¹³ is as defined in numbered paragraph (100) above.

In a particular group of compounds of Formula (I), or sub-formulae (I-I) to (I-XLV) defined herein, R¹³ is as defined in numbered paragraph (101) above.

In a particular group of compounds of Formula (I), or sub-formulae (I-I) to (I-XLV) defined herein, R¹⁴ is as defined in numbered paragraph (103) above.

In a particular group of compounds of Formula (I), or sub-formulae (I-I) to (I-XLV) defined herein, R¹⁴ is as defined in numbered paragraph (104) above.

In a particular group of compounds of Formula (I), or sub-formulae (I-I) to (I-XLV) defined herein, R¹⁴ is as defined in numbered paragraph (105) above.

In a particular group of compounds of Formula (I), or sub-formulae (I-I) to (I-XLV) defined herein, R^{a} is as defined in numbered paragraph (112) above.

In a particular group of compounds of Formula (I), or sub-formulae (I-I) to (I-XLV) defined herein, R^{a} is as defined in numbered paragraph (113) above.

In a particular group of compounds of Formula (I), or sub-formulae (I-I) to (I-XLV) defined herein, R^{a} is as defined in numbered paragraph (114) above.

Particular compounds of the present invention include any of the compounds exemplified in the present application, or a pharmaceutically acceptable salt or solvate thereof, and, in particular, any of the following:
(S)-(2-(Benzyloxy)-6-hydroxy-4-methoxyphenyl)(4-((tetrahydrofuran-3-yl)amino)isoindolin-2-yl)methanone
(R)-(2-(Benzyloxy)-6-hydroxy-4-methoxyphenyl)(4-((tetrahydrofuran-3-yl)amino)isoindolin-2-yl)methanone
(R)-(2-(Benzyloxy)-4-(difluoromethyl)-6-hydroxyphenyl)(4-((tetrahydrofuran-3-yl)amino)isoindolin-2-yl)methanone
(S)-(2-(Benzyloxy)-4-(difluoromethyl)-6-hydroxyphenyl)(4-((tetrahydrofuran-3-yl)amino)isoindolin-2-yl)methanone
(S)-(4-(Difluoromethyl)-2-hydroxy-6-((3-methoxybenzyl)oxy)phenyl)(4-((tetrahydrofuran-3-yl)amino)isoindolin-2-yl)methanone
(S)-(2-(Benzyloxy)-4-(difluoromethyl)-6-hydroxyphenyl)(6-((dimethylamino)methyl)-4-((tetrahydrofuran-3-yl)amino)isoindolin-2-yl)methanone
1-(3-((2-(2-(Benzyloxy)-4-(difluoromethyl)-6-hydroxybenzoyl)isoindolin-4-yl)amino)azetidin-1-yl)ethan-1-one
(2-(Benzyloxy)-4-(difluoromethyl)-6-hydroxyphenyl)(4-(oxetan-3-ylamino)isoindolin-2-yl)methanone
(4-(Difluoro methyl)-2-hydroxy-6-(pyridin-2-ylmethoxy) phenyl) (4-(oxetan-3-ylamino) isoindolin-2-yl) methanone
(S)-(2-(Benzyloxy)-4-(difluoromethyl)-6-hydroxyphenyl)(8-((tetrahydrofuran-3-yl)amino)-3,4-dihydroisoquinolin-2(1H)-yl)methanone
(2-(Benzyloxy)-4-(difluoromethyl)-6-hydroxyphenyl)(8-(oxetan-3-ylamino)-3,4-dihydroisoquinolin-2(1H)-yl) methanone
(S)-(4-(Difluoromethyl)-2-hydroxy-6-((4-methoxybenzyl)oxy)phenyl)(4-((tetrahydrofuran-3-yl)amino)isoindolin-2-yl)methanone
(S)-(4-(Difluoro methyl)-2-hydroxy-6-(pyridin-2-ylmethoxy) phenyl) (4-((tetrahydrofuran-3-yl) amino) isoindolin-2-yl) methanone
(S)-(4-(Difluoromethyl)-2-hydroxy-6-(pyridin-2-ylmethoxy)phenyl)(8-((tetrahydrofuran-3-yl)amino)-3,4-dihydroisoquinolin-2(1H)-yl)methanone
(S)-(4-(Difluoromethyl)-2-hydroxy-6-((4-methoxybenzyl)oxy)phenyl)(8-((tetrahydrofuran-3-yl)amino)-3,4-dihydroisoquinolin-2(1H)-yl)methanone
(S)-3-((2-(2-(Benzyloxy)-4-(difluoromethyl)-6-hydroxybenzoyl)isoindolin-4-yl)amino)-1-methylpyrrolidin-2-one
(S)-3-((2-(2-(benzyloxy)-4-(difluoromethyl)-6-hydroxybenzoyl)-1,2,3,4-tetrahydroisoquinolin-8-yl)amino)-1-methylpyrrolidin-2-one
(R)-3-((2-(2-(Benzyloxy)-4-(difluoromethyl)-6-hydroxybenzoyl)isoindolin-4-yl)amino)-1-methylpyrrolidin-2-one
(R)-3-((2-(2-(Benzyloxy)-4-(difluoromethyl)-6-hydroxybenzoyl)-1,2,3,4-tetrahydroisoquinolin-8-yl)amino)-1-methylpyrrolidin-2-one
3-((2-(2-(Benzyloxy)-4-(difluoromethyl)-6-hydroxybenzoyl)isoindolin-4-yl)amino)dihydrofuran-2(3H)-one
3-((2-(2-(Benzyloxy)-4-(difluoromethyl)-6-hydroxybenzoyl)-1,2,3,4-tetrahydroisoquinolin-8-yl) amino) dihydrofuran-2(3H)-one
(S)-(2-(Benzyloxy)-4-(difluoromethyl)-6-hydroxyphenyl)(6-methoxy-4-((tetrahydrofuran-3-yl)amino)isoindolin-2-yl)methanone
(S)-(6-((1H-Imidazol-1-yl) methyl)-4-((tetrahydrofuran-3-yl) amino) isoindolin-2-yl) (2-(benzyloxy)-4-(difluoromethyl)-6-hydroxyphenyl)methanone
(S)-(2-(Benzyloxy)-4-(difluoromethyl)-6-hydroxyphenyl)(6-methoxy-8-((tetrahydrofuran-3-yl)amino)-3,4-dihydroisoquinolin-2(1H)-yl)methanone
(S)-(2-(Benzyloxy)-4-(difluoromethyl)-6-hydroxyphenyl)(6-((dimethylamino)methyl)-8-((tetrahydrofuran-3-yl)amino)-3,4-dihydroisoquinolin-2(1H)-yl)methanone
(S)-(6-((1H-Imidazol-1-yl)methyl)-8-((tetrahydrofuran-3-yl)amino)-3,4-dihydroisoquinolin-2(1H)-yl)(2-(benzyloxy)-4-(difluoromethyl)-6-hydroxyphenyl)methanone
(S)-(2-(Benzyloxy)-4-(difluoromethyl)-6-hydroxyphenyl)(7-methoxy-8-((tetrahydrofuran-3-yl)amino)-3,4-dihydroisoquinolin-2(1H)-yl)methanone
(S)-(2-(Benzyloxy)-4-(difluoromethyl)-6-hydroxyphenyl)(6-(1-methyl-1H-imidazol-5-yl)-4-((tetrahydrofuran-3-yl)amino)isoindolin-2-yl)methanone
(S)-(2-(Benzyloxy)-4-(difluoromethyl)-6-hydroxyphenyl)(6-(1-methyl-1H-imidazol-4-yl)-4-((tetrahydrofuran-3-yl)amino)isoindolin-2-yl)methanone
(S)-(2-(Benzyloxy)-4-(difluoromethyl)-6-hydroxyphenyl) (6-(1-methyl-1H-imidazol-5-yl)-8-((tetrahydrofuran-3-yl) amino)-3,4-dihydroisoquinolin-2(1H)-yl)methanone
(S)-(2-(Benzyloxy)-4-(difluoromethyl)-6-hydroxyphenyl) (6-(1-methyl-1H-imidazol-4-yl)-8-((tetrahydrofuran-3-yl) amino)-3,4-dihydroisoquinolin-2(1H)-yl)methanone
2-(Benzyloxy)-4-(difluoromethyl)-6-hydroxyphenyl)(4-((2-methoxyethyl)amino)isoindolin-2-yl)methanone
2-((2-(2-(Benzyloxy)-4-(difluoro methyl)-6-hydroxybenzoyl) isoindolin-4-yl) amino)-N, N-dimethylacetamide
(2-(Benzyloxy)-4-(difluoromethyl)-6-hydroxyphenyl) (5-((4-methylpiperazin-1-yl) methyl) isoindolin-2-yl) methanone
(2-(Benzyloxy)-4-(difluoromethyl)-6-hydroxyphenyl)(5-(2-(dimethylamino)ethoxy)isoindolin-2-yl)methanone
(2-(Benzyloxy)-4-(difluoromethyl)-6-hydroxyphenyl)(5-((1-methylpiperidin-4-yl)oxy)isoindolin-2-yl)methanone
((2-(benzyloxy)-4-(difluoromethyl)-6-hydroxyphenyl)(4-((1-methylazetidin-3-yl)amino)isoindolin-2-yl)methanone
(2-(Benzyloxy)-4-(difluoro methyl)-6-hydroxyphenyl) (4-(methylamino) isoindolin-2-yl) methanone
(2-(Benzyloxy)-4-(difluoromethyl)-6-hydroxyphenyl)(3,4-dihydroisoquinolin-2(1H)-yl)methanone
(2-(Benzyloxy)-4-(difluoromethyl)-6-hydroxyphenyl)(8-(methylamino)-3,4-dihydroisoquinolin-2(1H)-yl)methanone
(2-(Benzyloxy)-4-(difluoromethyl)-6-hydroxyphenyl)(8-((2-methoxyethyl)amino)-3,4-dihydroisoquinolin-2(1H)-yl)methanone
(2-(Benzyloxy)-4-(difluoromethyl)-6-hydroxyphenyl)(7-methoxy-3,4-dihydroisoquinolin-2(1H)-yl)methanone
(2-(Benzyloxy)-4-(difluoromethyl)-6-hydroxyphenyl)(6-methoxy-3,4-dihydroisoquinolin-2(1H)-yl)methanone
(2-(Benzyloxy)-4-(difluoromethyl)-6-hydroxyphenyl)(7-(2-(dimethylamino)ethoxy)-3,4-dihydroisoquinolin-2(1H)-yl)methanone
(2-(Benzyloxy)-4-(difluoromethyl)-6-hydroxyphenyl)(6-(2-(dimethylamino)ethoxy)-3,4-dihydroisoquinolin-2(1H)-yl)methanone
(2-(Benzyloxy)-4-(difluoromethyl)-6-hydroxyphenyl)(7-((dimethylamino)methyl)-3,4-dihydroisoquinolin-2(1H)-yl)methanone
(2-(Benzyloxy)-4-(difluoromethyl)-6-hydroxyphenyl)(6-((dimethylamino)methyl)-3,4-dihydroisoquinolin-2(1H)-yl)methanone
(2-(Benzyloxy)-4-(difluoromethyl)-6-hydroxyphenyl)(7-((1-methylpiperidin-4-yl)oxy)-3,4-dihydroisoquinolin-2(1H)-yl)methanone
(2-(Benzyloxy)-4-(difluoromethyl)-6-hydroxyphenyl)(6-((1-methylpiperidin-4-yl)oxy)-3,4-dihydroisoquinolin-2(1H)-yl)methanone
(2-(Benzyloxy)-4-(difluoromethyl)-6-hydroxyphenyl)(7-((4-methylpiperazin-1-yl)methyl)-3,4-dihydroisoquinolin-2(1H)-yl)methanone
(2-(Benzyloxy)-4-(difluoromethyl)-6-hydroxyphenyl)(6-((4-methylpiperazin-1-yl)methyl)-3,4-dihydroisoquinolin-2(1H)-yl)methanone
(7-((1H-Imidazol-1-yl)methyl)-3,4-dihydroisoquinolin-2(1H)-yl)(2-(benzyloxy)-4-(difluoromethyl)-6-hydroxyphenyl)methanone
(6-((1H-Imidazol-1-yl)methyl)-3,4-dihydroisoquinolin-2(1H)-yl)(2-(benzyloxy)-4-(difluoromethyl)-6-hydroxyphenyl)methanone
(5-((1H-Imidazol-1-yl)methyl)isoindolin-2-yl)(2-(benzyloxy)-4-(difluoromethyl)-6-hydroxyphenyl)methanone
(2-(Benzyloxy)-4-(difluoromethyl)-6-hydroxyphenyl)(5-((dimethylamino)methyl)isoindolin-2-yl)methanone
(2-(Benzyloxy)-4-(difluoromethyl)-6-hydroxyphenyl)(2-((dimethylamino)methyl)-5,7-dihydro-6H-pyrrolo[3,4-b]pyridin-6-yl)methanone
(2-(Benzyloxy)-4-(difluoromethyl)-6-hydroxyphenyl)(3-((dimethylamino)methyl)-5,7-dihydro-6H-pyrrolo[3,4-b]pyridin-6-yl)methanone
(2-(Benzyloxy)-4-(difluoromethyl)-6-hydroxyphenyl)(isoindolin-2-yl)methanone
(R)-(2-(Benzyloxy)-4-(difluoromethyl)-6-hydroxyphenyl)(5-((1-methylpyrrolidin-3-yl)oxy)isoindolin-2-yl)methanone
(S)-(2-(Benzyloxy)-4-(difluoromethyl)-6-hydroxyphenyl)(5-((1-methylpyrrolidin-3-yl)oxy)isoindolin-2-yl)methanone
2-(Benzyloxy)-4-(difluoromethyl)-6-hydroxyphenyl)(5-((1-methylazetidin-3-yl)oxy)isoindolin-2-yl)methanone
(4-(Difluoromethyl)-2-hydroxy-6-(pyridin-2-ylmethoxy)phenyl)(5-((1-methylpiperidin-4-yl)oxy)isoindolin-2-yl)methanone
(2-(Cyclopropylmethoxy)-4-(difluoromethyl)-6-hydroxyphenyl)(5-((1-methylpiperidin-4-yl)oxy)isoindolin-2-yl)methanone
(2-(Benzyloxy)-4-(difluoromethyl)-6-hydroxyphenyl)(5-(pyridin-4-yloxy)isoindolin-2-yl)methanone
(2-(Cyclopropylmethoxy)-4-(difluoromethyl)-6-hydroxyphenyl)(5-((1-methylpiperidin-4-yl)oxy)isoindolin-2-yl)methanone
(S)-(2-(Benzyloxy)-4-(difluoromethyl)-6-hydroxyphenyl)(6-((1-methylpiperidin-4-yl)oxy)-4-((tetrahydrofuran-3-yl)amino)isoindolin-2-yl)methanone
(R)-(2-(Benzyloxy)-4-(difluoromethyl)-6-hydroxyphenyl)(6-((1-methylpiperidin-4-yl)oxy)-4-((tetrahydrofuran-3-yl)amino)isoindolin-2-yl)methanone
(S)-(2-(Benzyloxy)-4-(difluoromethyl)-6-hydroxyphenyl)(6-((1-methylpyrrolidin-3-yl)oxy)-3,4-dihydroisoquinolin-2(1H) yl)methanone
(R)-(2-(Benzyloxy)-4-(difluoromethyl)-6-hydroxyphenyl)(6-((1-methylpyrrolidin-3-yl)oxy)-3,4-dihydroisoquinolin-2(1H) yl)methanone
(2-(Benzyloxy)-4-(difluoromethyl)-6-hydroxyphenyl)(6-((1-methylazetidin-3-yl)oxy)-3,4-dihydroisoquinolin-2(1H)-yl)methanone
(R)-(2-(Benzyloxy)-4-(difluoromethyl)-6-hydroxyphenyl)(7-((1-methylpyrrolidin-3-yl)oxy)-3,4-dihydroisoquinolin-2(1H)-yl)methanone
(S)-(2-(Benzyloxy)-4-(difluoromethyl)-6-hydroxyphenyl)(7-((1-methylpyrrolidin-3-yl)oxy)-3,4-dihydroisoquinolin-2(1H)-yl)methanone
(2-(Benzyloxy)-4-(difluoromethyl)-6-hydroxyphenyl)(7-((1-methylazetidin-3-yl)oxy)-3,4-dihydroisoquinolin-2(1H)-yl)methanone
(2-(Benzyloxy)-4-(difluoro methyl)-6-hydroxyphenyl) (5-(2-morpholinoethoxy) isoindolin-2-yl) methanone
(2-(Benzyloxy)-4-(difluoromethyl)-6-hydroxyphenyl) (5-(2-(4-methylpiperazin-1-yl) ethoxy) isoindolin-2-yl) methanone
(2-(Benzyloxy)-4-(difluoromethyl)-6-hydroxyphenyl)(8-(cyclopentylamino)-6-(2-(dimethylamino)ethoxy)-3,4-dihydroisoquinolin-2(1H)-yl)methanone
(2-(Benzyloxy)-4-(difluoromethyl)-6-hydroxyphenyl)(6-(2-(dimethylamino)ethoxy)-8-(methylamino)-3,4-dihydroisoquinolin-2(1H)-yl)methanone
(2-(Benzyloxy)-4-(difluoromethyl)-6-hydroxyphenyl)(8-(difluoromethyl)-6-(2-(dimethylamino)ethoxy)-3,4-dihydroisoquinolin-2(1H)-yl)methanone
(R)-(2-(Benzyloxy)-4-(difluoromethyl)-6-hydroxyphenyl)(6-(2-(dimethylamino)ethoxy)-4-((tetrahydrofuran-3-yl)amino)isoindolin-2-yl)methanone
((S)-(2-(Benzyloxy)-4-(difluoromethyl)-6-hydroxyphenyl)(6-(2-(dimethylamino)ethoxy)-4-((tetrahydrofuran-3-yl)amino)isoindolin-2-yl)methanone
(2-(Benzyloxy)-4-(difluoromethyl)-6-hydroxyphenyl)(5-(2-methoxyethoxy)isoindolin-2-yl)methanone
(2-(Benzyloxy)-4-(difluoromethyl)-6-hydroxyphenyl)(5-((tetrahydro-2H-pyran-4-yl)oxy)isoindolin-2-yl)methanone
(2-(Benzyloxy)-4-(difluoromethyl)-6-hydroxyphenyl)(4-((tetrahydrofuran-3-yl)oxy)isoindolin-2-yl)methanone 2-(Benzyloxy)-4-(difluoromethyl)-6-hydroxyphenyl)(8-((tetrahydrofuran-3-yl)oxy)-3,4-dihydroisoquinolin-2(1H)-yl)methanone
(S)-(2-(Benzyloxy)-4-fluoro-6-hydroxyphenyl)(4-((tetrahydrofuran-3-yl)amino)isoindolin-2-yl)methanone
(R)-(2-(Benzyloxy)-4-fluoro-6-hydroxyphenyl)(4-((tetrahydrofuran-3-yl)amino)isoindolin-2-yl)methanone
(R)-(2-(Benzyloxy)-4-chloro-6-hydroxyphenyl)(4-((tetrahydrofuran-3-yl)amino)isoindolin-2-yl)methanone
(S)-(2-(Benzyloxy)-4-chloro-6-hydroxyphenyl)(4-((tetrahydrofuran-3-yl)amino)isoindolin-2-yl)methanone
(R)-(2-(Benzyloxy)-4-chloro-6-hydroxyphenyl)(6-(2-(dimethylamino)ethoxy)-4-((tetrahydrofuran-3-yl)amino)isoindolin-2-yl)methanone
(S)-(2-(Benzyloxy)-4-chloro-6-hydroxyphenyl)(6-(2-(dimethylamino)ethoxy)-4-((tetrahydrofuran-3-yl)amino)isoindolin-2-yl)methanone
(R)-(2-(Benzyloxy)-4-chloro-6-hydroxyphenyl)(6-methoxy-4-((tetrahydrofuran-3-yl)amino)isoindolin-2-yl)methanone
(S)-(2-(benzyloxy)-4-chloro-6-hydroxyphenyl)(6-methoxy-4-((tetrahydrofuran-3-yl)amino)isoindolin-2-yl)methanone
3-(Benzyloxy)-5-hydroxy-4-(4-((1-methyl-5-oxopyrrolidin-3-yl)amino)isoindoline-2-carbonyl)benzonitrile
3-(Benzyloxy)-5-hydroxy-4-(4-(pyrimidin-5-ylamino)isoindoline-2-carbonyl)benzonitrile
(R)-4-(4-((1-Acetylpyrrolidin-3-yl)amino)isoindoline-2-carbonyl)-3-(benzyloxy)-5-hydroxybenzonitrile
(S)-4-(4-((1-Acetylpyrrolidin-3-yl)amino)isoindoline-2-carbonyl)-3-(benzyloxy)-5-hydroxybenzonitrile
(R)-3-(Benzyloxy)-5-hydroxy-4-(4-((1-methyl-5-oxopyrrolidin-3-yl)amino)isoindoline-2-carbonyl)benzonitrile or (S)-3-(benzyloxy)-5-hydroxy-4-(4-((1-methyl-5-oxopyrrolidin-3-yl)amino)isoindoline-2-carbonyl)benzonitrile
3-(Benzyloxy)-5-hydroxy-4-(4-((oxazol-4-ylmethyl)amino)isoindoline-2-carbonyl)benzonitrile
3-(Benzyloxy)-5-hydroxy-4-(4-(((tetrahydrofuran-3-yl)methyl)amino)isoindoline-2-carbonyl)benzonitrile
3-(Benzyloxy)-5-hydroxy-4-(4-methoxyisoindoline-2-carbonyl)benzonitrile
(R)-3-(Benzyloxy)-5-hydroxy-4-(8-((tetrahydrofuran-3-yl)amino)-1,2,3,4-tetrahydroisoquinoline-2-carbonyl)benzonitrile
(R)-3-(benzyloxy)-5-hydroxy-4-(6-methoxy-4-((tetrahydrofuran-3-yl)amino)isoindoline-2-carbonyl)benzonitrile
(S)-3-(Benzyloxy)-5-hydroxy-4-(6-methoxy-4-((tetrahydrofuran-3-yl)amino)isoindoline-2-carbonyl)benzonitrile
3-(Benzyloxy)-4-(5-((dimethylamino)methyl)isoindoline-2-carbonyl)-5-hydroxybenzonitrile
4-(4-((1-Acetylpiperidin-4-yl)amino)isoindoline-2-carbonyl)-3-(benzyloxy)-5-hydroxybenzonitrile
(R)-4-(4-((1-Acetylpiperidin-3-yl)amino)isoindoline-2-carbonyl)-3-(benzyloxy)-5-hydroxybenzonitrile
(S)-4-(4-((1-Acetylpiperidin-3-yl)amino)isoindoline-2-carbonyl)-3-(benzyloxy)-5-hydroxybenzonitrile
3-(Benzyloxy)-5-hydroxy-4-(4-((5-oxotetrahydrofuran-3-yl)amino)isoindoline-2-carbonyl)benzonitrile
(2-(Benzyloxy)-4-chloro-6-hydroxyphenyl)(4-((1-methylazetidin-3-yl)amino)isoindolin-2-yl)methanone
2-(Benzyloxy)-4-chloro-6-hydroxyphenyl)(2-((dimethylamino)methyl)-5,7-dihydro-6H-pyrrolo[3,4-b]pyridin-6-yl)methanone
(2-(Benzyloxy)-4-chloro-6-hydroxyphenyl)(6-(2-(dimethylamino)ethoxy)-3,4-dihydroisoquinolin-2(1H)-yl)methanone
1-(3-((2-(2-(Benzyloxy)-4-fluoro-6-hydroxybenzoyl)isoindolin-4-yl)amino)azetidin-1-yl)ethan-1-one
(2-(Benzyloxy)-4-fluoro-6-hydroxyphenyl)(6-(2-(dimethylamino)ethoxy)-3,4-dihydroisoquinolin-2(1H)-yl)methanone
(2-(Benzyloxy)-4-fluoro-6-hydroxyphenyl) (5-((1-methylpiperidin-4-yl) oxy) isoindolin-2-yl) methadone
3-(Benzyloxy)-5-hydroxy-4-(4-(oxetan-3-ylamino)isoindoline-2-carbonyl)benzonitrile
(S)-3-(Benzyloxy)-5-hydroxy-4-(4-((1-methyl-2-oxopyrrolidin-3-yl)amino)isoindoline-2-carbonyl)benzonitrile
(R)-3-(benzyloxy)-5-hydroxy-4-(4-((1-methyl-2-oxopyrrolidin-3-yl)amino)isoindoline-2-carbonyl)benzonitrile
3-(Benzyloxy)-4-(6-(2-(dimethyl amino) ethoxy)-1,2,3,4-tetrahydroisoquinoline-2-carbonyl)-5-hydroxybenzonitrile
(R)-3-(Benzyloxy)-5-hydroxy-4-(5-((1-methylpyrrolidin-3-yl)oxy)isoindoline-2-carbonyl)benzonitrile
(S)-3-(Benzyloxy)-5-hydroxy-4-(5-((1-methylpyrrolidin-3-yl)oxy)isoindoline-2-carbonyl)benzonitrile
3-(Benzyloxy)-5-hydroxy-4-(5-((1-methylazetidin-3-yl)oxy)isoindoline-2-carbonyl)benzonitrile
3-(Benzyloxy)-5-hydroxy-4-(5-((1-methylpiperidin-4-yl)oxy)isoindoline-2-carbonyl)benzonitrile
3-(Benzyloxy)-5-hydroxy-4-(5-((4-methylpiperazin-1-yl)methyl)isoindoline-2-carbonyl)benzonitrile
3-(Benzyloxy)-5-hydroxy-4-(5-(2-methoxyethoxy)isoindoline-2-carbonyl)benzonitrile
3-(Benzyloxy)-5-hydroxy-4-(5-((tetrahydro-2H-pyran-4-yl)oxy)isoindoline-2-carbonyl)benzonitrile
3-Hydroxy-4-(5-((1-methylazetidin-3-yl)oxy)isoindoline-2-carbonyl)-5-(pyridin-2-ylmethoxy)benzonitrile
3-Hydroxy-4-(4-(oxetan-3-ylamino)isoindoline-2-carbonyl)-5-(pyridin-2-ylmethoxy)benzonitrile
(S)-3-Hydroxy-5-(pyridin-2-ylmethoxy)-4-(4-((tetrahydrofuran-3-yl)amino)isoindoline-2-carbonyl)benzonitrile
(S)-3-(Benzyloxy)-5-hydroxy-4-(4-((tetrahydrofuran-3-yl)amino)isoindoline-2-carbonyl)benzonitrile
(S)-3-Hydroxy-5-methoxy-4-(4-((tetrahydrofuran-3-yl)amino)isoindoline-2-carbonyl)benzonitrile
(S)-3-(Cyclopropylmethoxy)-5-hydroxy-4-(4-((tetrahydrofuran-3-yl)amino)isoindoline-2-carbonyl)benzonitrile
(R)-3-(Benzyloxy)-5-hydroxy-4-(4-((tetrahydrofuran-3-yl)amino)isoindoline-2-carbonyl)benzonitrile
3-(Benzyloxy)-5-hydroxy-4-(isoindoline-2-carbonyl)benzonitrile
3-(Benzyloxy)-5-hydroxy-4-(5-methoxyisoindoline-2-carbonyl)benzonitrile
(R)-3-(Benzyloxy)-4-(6-(2-(dimethylamino)ethoxy)-8-((tetrahydrofuran-3-yl)amino)-1,2,3,4-tetrahydroisoquinoline-2-carbonyl)-5-hydroxybenzonitrile
(S)-3-(Benzyloxy)-4-(6-(2-(dimethylamino)ethoxy)-8-((tetrahydrofuran-3-yl)amino)-1,2,3,4-tetrahydroisoquinoline-2-carbonyl)-5-hydroxybenzonitrile
4-(4-((1-Acetylazetidin-3-yl)amino)isoindoline-2-carbonyl)-3-(benzyloxy)-5-hydroxybenzonitrile
(S)-3-(Benzyloxy)-5-hydroxy-4-(8-((tetrahydrofuran-3-yl)amino)-1,2,3,4-tetrahydroisoquinoline-2-carbonyl)benzonitrile
(R)-3-(Benzyloxy)-4-(6-(2-(dimethylamino)ethoxy)-4-((tetrahydrofuran-3-yl)amino)isoindoline-2-carbonyl)-5-hydroxybenzonitrile
(S)-3-(Benzyloxy)-4-(6-(2-(dimethylamino)ethoxy)-4-((tetrahydrofuran-3-yl)amino)isoindoline-2-carbonyl)-5-hydroxybenzonitrile
(R)-(2-(Benzyloxy)-4-chloro-6-hydroxyphenyl)(6-(2-(dimethylamino)ethoxy)-8-((tetrahydrofuran-3-yl)amino)-3,4-dihydroisoquinolin-2(1H)-yl)methanone
(S)-(2-(Benzyloxy)-4-chloro-6-hydroxyphenyl)(6-(2-(dimethylamino)ethoxy)-8-((tetrahydrofuran-3-yl)amino)-3,4-dihydroisoquinolin-2(1H)-yl)methanone
(S)-(2-(Benzyloxy)-6-hydroxy-4-(trifluoromethyl) phenyl) (4-((tetrahydrofuran-3-yl) amino) isoindolin-2-yl) methanone
(S)-(2-(Benzyloxy)-4-(difluoromethyl)-6-hydroxyphenyl)(6-(2-(dimethylamino)ethoxy)-8-((tetrahydrofuran-3-yl)amino)-3,4-dihydroisoquinolin-2(1H)-yl)methanone
(R)-(2-(Benzyloxy)-4-(difluoromethyl)-6-hydroxyphenyl)(6-(2-(dimethylamino)ethoxy)-8-((tetrahydrofuran-3-yl)amino)-3,4-dihydroisoquinolin-2(1H)-yl)methanone
(2-(Benzyloxy)-4-(difluoromethyl)-6-hydroxyphenyl)(5-(piperidin-4-yloxy)isoindolin-2-yl)methanone
3-(Benzyloxy)-4-(6-((dimethylamino)methyl)-2,3-dihydro-1H-pyrrolo[3,4-c]pyridine-2-carbonyl)-5-hydroxybenzonitrile
3-(Benzyloxy)-5-hydroxy-4-(6-(4-methylpiperazin-1-yl)-2,3-dihydro-1H-pyrrolo[3,4-c]pyridine-2-carbonyl)benzonitrile
(2-(Benzyloxy)-4-(difluoromethyl)-6-hydroxyphenyl)(6-((tetrahydrofuran-3-yl)amino)indolin-1-yl)methanone
(S or R)-(2-(Benzyloxy)-4-(difluoromethyl)-6-hydroxyphenyl)(6-((tetrahydrofuran-3-yl)amino)indolin-1-yl)methanone
3-(Benzyloxy)-5-hydroxy-4-(6-((tetrahydrofuran-3-yl)amino)indoline-1-carbonyl)benzonitrile
3-(Benzyloxy)-5-hydroxy-4-(5-((tetrahydrofuran-3-yl)amino)indoline-1-carbonyl)benzonitrile
(2-(Benzyloxy)-4-(difluoromethyl)-6-hydroxyphenyl)(5-((tetrahydrofuran-3-yl)amino)indolin-1-yl)methanone
(S or R)-(2-(Benzyloxy)-4-(difluoromethyl)-6-hydroxyphenyl)(5-((tetrahydrofuran-3-yl)amino)indolin-1-yl)methanone
3-(Benzyloxy)-5-hydroxy-4-(7-((1-methylpiperidin-4-yl)oxy)-2,3,4,5-tetrahydro-1H-benzo[d]azepine-3-carbonyl)benzonitrile hydroformate
(2-(Benzyloxy)-4-(difluoromethyl)-6-hydroxyphenyl)(7-((1-methylpiperidin-4-yl)oxy)-1,2,4,5-tetrahydro-3H-benzo[d]azepin-3-yl)methanone
3-(Benzyloxy)-5-hydroxy-4-(6-morpholino-2,3-dihydro-1H-pyrrolo[3,4-c]pyridine-2-carbonyl)benzonitrile
(2-(Benzyloxy)-4-(difluoromethyl)-6-hydroxyphenyl)(6-morpholino-1,3-dihydro-2H-pyrrolo[3,4-c]pyridin-2-yl)methanone
4-(6-Amino-2,3-dihydro-1H-pyrrolo[3,4-c]pyridine-2-carbonyl)-3-(benzyloxy)-5-hydroxybenzonitrile hydroformate
(6-Amino-1,3-dihydro-2H-pyrrolo[3,4-c]pyridin-2-yl)(2-(benzyloxy)-4-(difluoromethyl)-6-hydroxyphenyl)methanone hydroformate
3-(Benzyloxy)-4-(4-(2-(dimethylamino)ethoxy)indoline-1-carbonyl)-5-hydroxybenzonitrile
(2-(Benzyloxy)-4-(difluoromethyl)-6-hydroxyphenyl)(4-(2-(dimethylamino)ethoxy)indolin-1-yl)methanone
3-(Benzyloxy)-4-(2,3-dihydro-1H-pyrrolo[2,3-b]pyridine-1-carbonyl)-5-hydroxybenzonitrile
(2-(Benzyloxy)-4-(difluoromethyl)-6-hydroxyphenyl)(2,3-dihydro-1H-pyrrolo[2,3-b]pyridin-1-yl)methanone
3-(Benzyloxy)-4-(2-(2-(dimethylamino)ethoxy)-4-((tetrahydrofuran-3-yl)amino)-5,6,7,8-tetrahydropyrido[4,3-d]pyrimidine-6-carbonyl)-5-hydroxybenzonitrile
(2-(Benzyloxy)-4-(difluoromethyl)-6-hydroxyphenyl)(2-(2-(dimethylamino)ethoxy)-4-((tetrahydrofuran-3-yl)amino)-7,8-dihydropyrido[4,3-d]pyrimidin-6(5H)-yl)methanone
3-(Benzyloxy)-5-hydroxy-4-(6-((tetrahydrofuran-3-yl)amino)-1,2,3,4-tetrahydroquinoline-1-carbonyl)benzonitrile
(2-(Benzyloxy)-4-(difluoromethyl)-6-hydroxyphenyl)(6-((tetrahydrofuran-3-yl)amino)-3,4-dihydroquinolin-1(2H)-yl)methanone
(2-(Benzyloxy)-4-(difluoromethyl)-6-hydroxyphenyl)(4-((tetrahydrofuran-3-yl)amino)-7,8-dihydropyrido[4,3-d]pyrimidin-6(5H)-yl)methanone
(2-(Benzyloxy)-4-(difluoromethyl)-6-hydroxyphenyl)(2,3-dihydro-1H-pyrrolo[3,2-c]pyridin-1-yl)methanone
(2-(Benzyloxy)-4-(difluoromethyl)-6-hydroxyphenyl)(2,3-dihydro-1H-pyrrolo[3,2-b]pyridin-1-yl)methanone
(2-(Benzyloxy)-4-(difluoromethyl)-6-hydroxyphenyl)(2,3-dihydro-1H-pyrrolo[2,3-c]pyridin-1-yl)methanone
(2-(Benzyloxy)-4-(difluoromethyl)-6-hydroxyphenyl)(4-((tetrahydrofuran-3-yl)amino)indolin-1-yl)methanone
(S)-(4-(Difluoromethyl)-2-hydroxy-6-((1-methyl-1H-indazol-6-yl)methoxy)phenyl)(8-((tetrahydrofuran-3-yl)amino)-3,4-dihydroisoquinolin-2(1H)-yl)methanone
(S)-(2-((2-Oxabicyclo[2.1.1]hexan-1-yl)methoxy)-4-(difluoromethyl)-6-hydroxyphenyl)(8-((tetrahydrofuran-3-yl)amino)-3,4-dihydroisoquinolin-2(1H)-yl)methanone
(S)-(4-(Difluoromethyl)-2-hydroxy-6-(isoquinolin-6-ylmethoxy)phenyl)(8-((tetrahydrofuran-3-yl)amino)-3,4-dihydroisoquinolin-2(1H)-yl)methanone
(S)-(4-(Difluoromethyl)-2-hydroxy-6-(isoquinolin-7-ylmethoxy)phenyl)(8-((tetrahydrofuran-3-yl)amino)-3,4-dihydroisoquinolin-2(1H)-yl)methanone
(S)-(2-((1H-Benzo[d]imidazol-2-yl)methoxy)-4-(difluoromethyl)-6-hydroxyphenyl)(8-((tetrahydrofuran-3-yl)amino)-3,4-dihydroisoquinolin-2(1H)-yl)methanone.

1-(4-((2-(2-(Benzyloxy)-4-(difluoro methyl)-6-hydroxybenzoyl) isoindolin-5-yl) oxy) piperidin-1-yl) ethan-1-one The various functional groups and substituents making up the compounds of the Formula (I), or sub-formulae (I-I) to (I-XLV), are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers).

It is to be understood that the present invention encompasses all optical, diastereoisomers and geometric isomers and mixtures thereof that possess activity.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 16O and 18O; and the like.

It is also to be understood that certain compounds of the Formula (I), or sub-formulae (I-I) to (I-XLV), may exist in solvated as well as unsolvated forms such as, for example, hydrated forms. It is to be understood that the invention encompasses all such solvated forms that possess activity.

It is also to be understood that certain compounds of the Formula (I), or sub-formulae (I-I) to (I-XLV), may exhibit polymorphism, and that the invention encompasses all such forms that possess activity.

Compounds of the Formula (I), or sub-formulae (I-I) to (I-XLV), may exist in a number of different tautomeric forms and references to compounds of the Formula (I), or sub-formulae (I-I) to (I-XLV), include all such forms. For the avoidance of doubt, where a compound can exist in one of several tautomeric forms, and only one is specifically described or shown, all others are nevertheless embraced by Formula (I), or sub-formulae (I-I) to (I-XLV). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

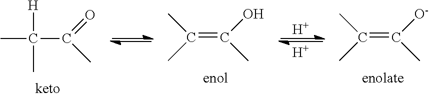

Compounds of the Formula (I), or sub-formulae (I-I) to (I-XLV), containing an amine function may also form N-oxides. A reference herein to a compound of the Formula (I), or sub-formulae (I-I) to (I-XLV), that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of Formula (I), or sub-formulae (I-I) to (I-XLV), may be administered in the form of a pro-drug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula (I), or sub-formulae (I-I) to (I-XLV), and in-vivo cleavable amide derivatives that may be formed at a carboxy group or an amino group in a compound of the Formula (I), or sub-formulae (I-I) to (I-XLV).

Accordingly, the present invention includes those compounds of the Formula (I), or sub-formulae (I-I) to (I-XLV), as defined hereinbefore, when made available by organic synthesis and when made available within the human or animal body by way of cleavage of a pro-drug thereof. Accordingly, the present invention includes those compounds of the Formula (I), or sub-formulae (I-I) to (I-XLV), that are produced by organic synthetic means and also such compounds that are produced in the human or animal body by way of metabolism of a precursor compound, that is a compound of the Formula (I), or sub-formulae (I-I) to (I-XLV), may be a synthetically-produced compound or a metabolically-produced compound.

A suitable pharmaceutically acceptable pro-drug of a compound of the Formula (I), or sub-formulae (I-I) to (I-XLV), is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the Formula (I), or sub-formulae (I-I) to (I-XLV), that possesses a carboxy group is, for example, an in vivo cleavable ester thereof. An in vivo cleavable ester of a compound of the Formula (I), or sub-formulae (I-I) to (I-XLV), containing a carboxy group is, for example, a pharmaceutically acceptable ester which is cleaved in the human or animal body to produce the parent acid or parent alcohol. Suitable pharmaceutically acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically acceptable pro-drug of a compound of the Formula (I), or sub-formulae (I-I) to (I-XLV), that possesses a hydroxy group is, for example, an in vivo cleavable ester or ether thereof. An in vivo cleavable ester or ether of a compound of the Formula (I), or sub-formulae (I-I) to (I-XLV), containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-(1-6C)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the Formula (I), or sub-formulae (I-I) to (I-XLV), that possesses a carboxy group is, for example, an in vivo cleavable amide thereof, for example an amide formed with an amine such as ammonia, a (1-4C)alkylamine such as methylamine, a [(1-4C)alkyl]₂amine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically acceptable pro-drug of a compound of the Formula (I), or sub-formulae (I-I) to (I-XLV), that possesses an amino group is, for example, an in vivo cleavable amide derivative thereof. Suitable pharmaceutically acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkyl)piperazin-1-ylmethyl.

The in vivo effects of a compound of the Formula (I), or sub-formulae (I-I) to (I-XLV), may be exerted in part by one or more metabolites that are formed within the human or animal body after administration of a compound of the Formula (I), or sub-formulae (I-I) to (I-XLV). As stated hereinbefore, the in vivo effects of a compound of the Formula (I), or sub-formulae (I-I) to (I-XLV), may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The methodology employed to synthesise a compound of Formula (I), or sub-formulae (I-I) to (I-XLV), will vary depending on the nature of any substituent groups or subgroups associated therewith. Suitable processes for their preparation are described further in the accompanying Examples.

Once a compound of Formula (I), or sub-formulae (I-I) to (I-XLV), has been synthesised by any one of the processes defined herein, the processes may then further comprise the additional steps of:
(i) removing any protecting groups present;
(ii) converting the compound Formula (I) into another compound of Formula (I);
(iii) forming a pharmaceutically acceptable salt, hydrate or solvate thereof; and/or
(iv) forming a prodrug thereof.

An example of (ii) above is when a compound of Formula (I) is synthesised and then one or more of the groups R², R³, R⁴, R⁵, R¹¹, R¹², R¹³ or R¹⁴ may be further reacted to change the nature of the group and provide an alternative compound of Formula (I).

The resultant compounds of Formula (I), or sub-formulae (I-I) to (I-XLV), can be isolated and purified using techniques well known in the art.

The compounds of Formula (I) may be synthesised by the synthetic routes shown in the Examples section below.

### Biological Activity

The biological assays described in the Examples section herein may be used to measure the pharmacological effects of the compounds of the present invention.

Although the pharmacological properties of the compounds of Formula (I) vary with structural change, as expected, the compounds of the invention were found to be active in the MLH1 in vitro assay described in the Example section.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula (I) will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as inhibitors of MLH1 activity.

The compounds of Formula (I), or a pharmaceutically acceptable salt thereof, therefore have potential therapeutic uses in a variety of disease states in which the inhibition of MLH1 activity is beneficial.

The present invention therefore provides a method of treating a disease or disorder in which the inhibition MLH1 activity is beneficial in a patient in need of such treatment, said method comprising administering to said patient a therapeutically effective amount of a compound, or a pharmaceutically acceptable salt, hydrate or solvate thereof, or a pharmaceutical composition as defined herein.

The present invention provides a method of inhibiting MLH1 activity, in vitro or in vivo, said method comprising contacting a cell with an effective amount of a compound or a pharmaceutically acceptable salt, hydrate or solvate thereof as defined herein.

The present invention provides a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein.

The present invention provides a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein.

The present invention provides a compound, or a pharmaceutically acceptable salt, hydrate or solvate thereof, or a pharmaceutical composition as defined herein for use in therapy.

The present invention provides a compound, or a pharmaceutically acceptable salt, hydrate or solvate thereof, or a pharmaceutical composition as defined herein for use as a medicament.

The present invention provides a compound or a pharmaceutically acceptable salt, hydrate or solvate thereof as defined herein, or a pharmaceutical composition as defined herein, for use in the treatment of a proliferative disorder.

The present invention provides a compound, or a pharmaceutically acceptable salt, hydrate or solvate thereof, or a pharmaceutical composition as defined herein for use in the treatment of cancer. In a particular embodiment, the cancer is human cancer. In a particular embodiment, the cancer is human cancer, in particular oestrogen positive cancers, such as breast cancer, or androgen receptor positive cancers, such as prostate cancer.

The present invention provides a compound, or a pharmaceutically acceptable salt, hydrate or solvate thereof, as defined herein for use in the inhibition of MLH1 activity.

The present invention provides a compound, or a pharmaceutically acceptable salt, hydrate or solvate thereof, as defined herein for use in the treatment of a disease or disorder in which the inhibition of MLH1 activity is beneficial.

The present invention provides a use of a compound, or a pharmaceutically acceptable salt, hydrate or solvate thereof, as defined herein in the manufacture of a medicament for the treatment of a proliferative disorder.

The present invention provides a use of a compound, or a pharmaceutically acceptable salt, hydrate or solvate thereof, as defined herein in the manufacture of a medicament for the treatment of cancer.

The present invention provides a use of a compound, or a pharmaceutically acceptable salt, hydrate or solvate thereof, as defined herein in the manufacture of a medicament for the inhibition of MLH1 activity.

The present invention provides a use of a compound, or a pharmaceutically acceptable salt, hydrate or solvate thereof, as defined herein in the manufacture of a medicament for the treatment of a disease or disorder in which the inhibition of MLH1 activity is beneficial.

The term “proliferative disorder”, “proliferative condition” and “proliferative disease” are used interchangeably herein and pertain to an unwanted or uncontrolled cellular proliferation of excessive or abnormal cells which is undesired, such as, neoplastic or hyperplastic growth, whether in vitro or in vivo.

In the above-outlined aspects of the invention, the proliferative disorder is suitably cancer, and the cancer is suitably a human cancer. In the aspects of the invention outlined herein, the proliferative disorder is suitably cancer, and the cancer is suitably a human cancer. In particular, the compounds of the present invention will be useful for the treatment of any cancer in which mis-match repair inhibition and/or cGAS/STING pathway activation is beneficial. Any suitable cancer may be targeted (e.g. adenoid cystic carcinoma, adrenal gland tumor, amyloidosis, anal cancer, appendix cancer, astrocytoma, ataxia-telangiectasia, Beckwith-Wiedemann Syndrome, bile duct cancer (cholangiocarcinoma), Birt-Hogg-Dube Syndrome, bladder cancer, bone cancer, brain stem glioma, brain tumor, breast cancer, Carney Complex, central nervous system tumors, cervical cancer, colorectal cancer, Cowden Syndrome, craniopharyngioma, desmoplastic infantile ganglioglioma, ependymoma, esophageal cancer, Ewing sarcoma, eye cancer, eyelid cancer, familial adenomatous polyposis, familial GIST, familial malignant melanoma, familial non-VHL clear cell renal cell carcinoma, familial pancreatic cancer, gallbladder cancer, gastrointestinal stromal tumor—GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, juvenile polyposis syndrome, kidney cancer, lacrimal gland tumor, laryngeal and hypopharyngeal cancer, leukemia (acute lymphoblastic leukamia (ALL), acute myeloid leukemia (AML), B-cell prolymphocytic leukemia, hairy cell leukemia, chronic lymphocytic leukemia (CLL), chronic myeloid leukemia (CML), chronic T-cell lymphocytic leukemia, eosinophilic leukemia), Li-Fraumeni Syndrome, liver cancer, lung cancer (non-small cell lung cancer, small cell lung cancer), Lymphoma (Hodgkin, non-Hodgkin), Lynch Syndrome, mastocytosis, medulloblastoma, melanoma, meningioma, mesothelioma, multiple endocrine neoplasia Type 1 & 2, multiple myeloma, MUTYH (or MYH)-associated polyposis, myelodysplastic syndromes (MDS), nasal cavity and paranasal sinus Cancer, nasopharyngeal Cancer, neuroblastoma, neuroendocrine tumors (e.g. of the gastrointestinal tract, lung or pancreas), neurofibromatosis Type 1 & 2, nevoid basal cell carcinoma syndrome, oral and oropharyngeal cancer, osteosarcoma, ovarian/fallopian tube/peritoneal cancer, pancreatic cancer, parathyroid cancer, penile cancer, Peutz-Jeghers Syndrome, pheochromocytoma, paraganglioma, pituitary gland tumor, pleuropulmonary blastoma, prostate cancer, retinoblastoma, rhabdomyosarcoma, salivary gland cancer, sarcoma (e.g. Kaposi or soft tissue), skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom's macroglobulinemia, Werner syndrome, Wilms Tumor and xeroderma pigmentosum). Particular cancers of interest include haematological cancers such as lymphomas (including diffuse large B-cell lymphoma (DLBCL), follicular lymphoma (FL), Burkitt lymphoma (BL) and angioimmunoblastic T-cell lymphoma (AITL)), leukaemias (including acute lymphoblastic leukaemia (ALL) and chronic myeloid leukaemia (CML)), multiple myeloma, breast cancer, non-small cell lung cancer (NSCLC), colorectal cancer, endometrial cancer, gastro-oesophageal cancer, neuroendocrine cancers, osteosarcomas, prostate cancer, pancreatic cancer, small intestine cancer, bladder cancer, rectal cancer, cholangiocarcinoma, CNS cancer, thyroid cancer, head and neck cancer, oesophageal cancer, and ovarian cancer.

The compounds of the present invention may also be used to treat triplet diseases.

Thus, a further aspect of the present invention provides a method of treating a triplet disorder (e.g. Huntington's disease (HD), myotonic dystrophy type 1 (DM1), fragile X syndrome type A (FRAXA), Friedreich's ataxia (FRDA), and spinocerebellar ataxias (SCAs)) in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a compound, or a pharmaceutically acceptable salt, hydrate or solvate thereof, or a pharmaceutical composition as defined herein for use in the treatment of a triplet disorder. In a particular embodiment, the triplet disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

According to a further aspect of the present invention, there is provided the use of a compound, or a pharmaceutically acceptable salt, hydrate or solvate thereof, as defined herein in the manufacture of a medicament for the treatment of a triplet disorder. In a particular embodiment, the triplet disorder is selected from the group consisting of Huntington's disease (HD), myotonic dystrophy type 1 (DM1), fragile X syndrome type A (FRAXA), Friedreich's ataxia (FRDA), and spinocerebellar ataxias (SCAs).

### Routes of Administration

The compounds of the invention or pharmaceutical compositions comprising these compounds may be administered to a subject by any convenient route of administration, whether systemically, peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including intratumoral, subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of the present invention may be administered as a sole therapy or may involve, in addition to a compound of the invention, conventional surgery or radiotherapy or chemotherapy or a targeted agent. Such chemotherapy or targeted agent may include one or more of the following categories:
(i) Antiproliferative/antineoplastic drugs and combinations thereof, as used in medical oncology, such as, but not limited to, alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);
(ii) cytostatic agents such as, but not limited to, antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), steroid hormones, including progestogens (for example megestrol acetate) and corticosteroids (for example dexamethasone, prednisone and prednisolone), aromatase inhibitors (for example as anastrozole, letrozole, vorazole and exemestane) and inhibitors of 5α-reductase such as finasteride;
(iii) anti-invasion agents such as, but not limited to, c-Src kinase family inhibitors 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), bosutinib (SKI-606), and metalloproteinase inhibitors such as marimastat, inhibitors of urokinase plasminogen activator receptor function or antibodies to Heparanase;
(iv) inhibitors of growth factor function such as, but not limited to, growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors and cyclin dependent kinase inhibitors such as CDK2 and/or CDK4 inhibitors;
(v) antiangiogenic agents such as, but not limited to, those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU11248), axitinib (AG-013736) and pazopanib (GW 786034).
(vi) vascular damaging agents such as, but not limited to, Combretastatin A₄ and compounds disclosed in International Patent Applications WO 99/02166, WO 00/40529, WO 00/41669, WO 01/92224, WO 02/04434 and WO 02/08213;
(vii) an endothelin receptor antagonist, for example zibotentan (ZD4054) or atrasentan;
(viii) antisense therapies, such as, but not limited to, those directed to targets listed above, such as ISIS 2503, an anti-ras antisense;
(ix) immunotherapy approaches, including for example cancer vaccines, antibody, viral (oncolytic viruses) and small molecule or cell therapy approaches to increase the immunogenicity of patient tumour cells and/or facilitate a cell mediated anti-tumour response. Such therapies could include, but are not limited to, OX40 agonists, cGAS-STING agonists, A₂a receptor antagonists, PI3 kinase inhibitors, TLR7/8 agonists, IDO inhibitors, Arginase inhibitors, BTK inhibitors and Bromodomain inhibitors; transduction with microbial vectors of cancer antigens, direct transduction of cancer antigens into antigen presenting cells, treatment with immune cells specific for cancer antigens (e.g. CAR-T), treatment with antibodies, antibody fragments and antibody drug conjugates that enable the immune system to recognise tumour cells.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt or solvate thereof, and an anti-tumour agent.

According to this aspect of the invention there is provided a combination for use in the treatment of a proliferative condition, such as cancer (for example a cancer involving a solid tumour), comprising a compound of the invention as defined hereinbefore, or a pharmaceutically acceptable salt or solvate thereof, and any one of the anti-tumour agents listed herein above.

In a further aspect of the invention there is provided a compound of the invention or a pharmaceutically acceptable salt or solvate thereof, for use in the treatment of cancer in combination with another anti-tumour agent, optionally selected from one listed herein above.

In a further aspect of the invention there is provided a compound of the invention or a pharmaceutically acceptable salt or solvate thereof, for use in the treatment of cancer in combination with a tyrosine kinase inhibitor, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt or solvate thereof, in combination with an anti-tumour agent (optionally selected from one listed herein above), in association with a pharmaceutically acceptable diluent or carrier.

Combination Therapy with Immune Modulating Treatments

### Immune Checkpoint Inhibitors

Immune checkpoint proteins present on immune cells and/or cancer cells [e.g. CTLA4 (also known as cytotoxic T-lymphocyte-associated protein 4 and CD152), LAG3 (also known as lymphocyte-activation gene 3 and CD223), PD1 (also known as programmed cell death protein 1 and CD279), PD-L1 (also known as programmed death-ligand 1 and CD274), TIM-3 (also known as T-cell immunoglobulin mucin-3) and TIGIT (also known as T-cell Immunoreceptor with Ig and ITIM domains) are molecular targets that have been found to play an important role in regulating anti-tumour immune responses. Inhibitors of these immune checkpoint proteins (e.g. CTLA4, LAG3, PD1, PD-L1, TIM-3 and/or TIGIT inhibitors) promote an anti-tumour immune response that can be utilised to effectively treat certain forms of cancer.

### Immune Stimulators

Monoclonal antibodies, bispecific antibodies, recombinant ligands and small molecule therapeutics that bind to stimulatory receptors on immune cells can facilitate an effective anti-tumour response. Such receptors may be involved in cell-to-cell contact for example contact between tumour cell and immune cell or between two types of immunce cells, other receptors may bind to soluble factors that stimulate an immune response. In one such embodiment antibodies, bispecifics, recombinant proteins or small molecule therapeutics can activate stimulatory receptors, including, but not limited to, 4-1BB, OX40, cGAS-STING, CD27, CD40, and DR3 that enhance anti-tumour immunity.

Modulators of antigen processing may facilitate the presentation of neoantigenic peptides on the cell surface to enhance an effective anti-tumour response. In one such embodiment inhibitors of the endoplasmic reticulum aminopeptidases ERAP1 and ERAP2 may stimulate anti-tumour immunity.

In one aspect, the present invention relates to a combination comprising a compound as defined herein, or a pharmaceutically acceptable salt thereof, and an immune checkpoint inhibitor or immune stimulator as defined herein, or a pharmaceutically acceptable salt thereof, for use in the treatment of a proliferative disorder.

In another aspect, the present invention relates to a use of a combination comprising a compound as defined herein, or a pharmaceutically acceptable salt thereof, and an immune checkpoint inhibitor or immune stimulator as defined herein, or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and an immune checkpoint inhibitor or immune stimulator as defined herein, or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administration with an immune checkpoint inhibitor, or immune stimulator, or a pharmaceutically acceptable salt thereof.

In another aspect, the present invention relates to an immune checkpoint inhibitor or immune stimulator, or a pharmaceutically acceptable salt thereof, for use in the treatment of a proliferative disorder, wherein the immune checkpoint inhibitor is for simultaneous, separate or sequential administration with a compound as defined herein, or a pharmaceutically acceptable salt thereof, as defined herein.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administration with an immune checkpoint inhibitor or immune stimulator, or a pharmaceutically acceptable salt thereof.

In another aspect, the present invention relates to a use of an immune checkpoint inhibitor or immune stimulator, or a pharmaceutically acceptable salt thereof, in the manufacture of a medicament for treating a proliferative disorder, wherein the medicament is for simultaneous, separate or sequential administration with a compound as defined herein, or a pharmaceutically acceptable salt thereof.

In another aspect, the present invention relates to a method of treating a proliferative disorder comprising administering to a subject in need thereof a therapeutically effective amount of a compound as defined herein, or a pharmaceutically acceptable salt thereof, as defined herein and an immune checkpoint inhibitor or immune stimulator as defined herein, or a pharmaceutically acceptable salt thereof, either sequentially, separately or simultaneously.

Any immune checkpoint inhibitor or immune stimulator may be used in the combination therapy defined herein.

In one embodiment, the immune stimulator is selected from a 4-1BB stimulator, a OX40 stimulator, a CD27 stimulator, a CD40 stimulator, and a DR3 stimulator. In another embodiment the immune checkpoint inhibitor is selected from a PD1-inhibitor, a PD-L1 inhibitor, a LAG3 inhibitor, CTLA-4 inhibitor, a TIM-3 inhibitor and/or a TIGIT inhibitor. In a particular embodiment, the immune checkpoint inhibitor is a PD1 or PD-L1 inhibitor.

PD-1 is a cell surface receptor protein present on immune cells such as T cells. PD-1 plays an important role in down-regulating the immune system and promoting self-tolerance by suppressing T cell activation. The PD-1 protein is an immune checkpoint that guards against autoimmunity through a dual mechanism of promoting apoptosis (programmed cell death) in antigen specific T cells in lymph nodes, while simultaneously reducing apoptosis in regulatory T cells (anti-inflammatory suppressive T cells).

PD-1 therefore inhibits the immune system. This prevents autoimmune diseases, but it can also prevent the immune system from killing cancer cells.

PD1 binds two ligands, PD-L1 and PD-L2. PD-L1 is of particular interest as it is highly expressed in several cancers and hence the role of PD1 in cancer immune evasion is well established. Monoclonal antibodies targeting PD-1 that boost the immune system are approved or are being developed for the treatment of cancer. Many tumour cells express PD-L1, an immunosuppressive PD-1 ligand; inhibition of the interaction between PD-1 and PD-L1 can enhance T-cell responses in vitro and mediate preclinical antitumour activity. This is known as immune checkpoint blockade.

Examples of drugs that target PD-1 include pembrolizumab (Keytruda) and nivolumab (Opdivo). These drugs have been shown to be effective in treating several types of cancer, including melanoma of the skin, non-small cell lung cancer, kidney cancer, bladder cancer, head and neck cancers, and Hodgkin lymphoma. They are also being studied for use against many other types of cancer. Examples of drugs in development include BMS-936559 (Bristol Myers Squibb), MGA012 (MacroGenics) and MEDI-0680 (MedImmune).

Examples of drugs that inhibit PD-L1 include atezolizumab (Tecentriq), avelumab (Bavencio) and durvalumab (Imfinzi). These drugs have also been shown to be helpful in treating different types of cancer, including bladder cancer, non-small cell lung cancer, and Merkel cell skin cancer (Merkel cell carcinoma). They are also being studied for use against other types of cancer.

Examples of LAG3 inhibitors include BMS-986016/Relatlimab, TSR-033, REGN3767, MGD013 (bispecific DART binding PD-1 and LAG-3), GSK2831781 and LAG525.

Examples of CTLA-4 inhibitors include MDX-010/Ipilimumab, AGEN1884, and CP-675,206/Tremelimumab.

Examples of TIM-3 inhibitors include MBG453 (Novartis), TSR-022 (Tesaro), and LY3321367 (Lilly).

Examples of TIGIT inhibitors include Tiragolumab (MTIG7192A; RG6058; Genentech/Roche), AB154 (Arcus Bioscience), MK-7684 (Merck), BMS-986207 (Bristol-Myers Squibb), ASP8374 (Astellas Pharma; Potenza Therapeutics).

In one embodiment, the immune checkpoint inhibitor is selected from BMS-986016/Relatlimab, TSR-033, REGN3767, MGD013 (bispecific DART binding PD-1 and LAG-3), GSK2831781, LAG525, MDX-010/Ipilimumab, AGEN1884, and CP-675,206/Tremelimumab, pembrolizumab, nivolumab, atezolizumab, avelumab, durvalumab, MBG453, TSR-022, LY3321367, Tiragolumab (MTIG7192A; RG6058), AB154, MK-7684, BMS-986207, and/or ASP8374 or a pharmaceutically acceptable salt or solvate thereof.

Combination Therapy with DNA Damage Response Modulators

The compounds of the present invention are particularly suited to use in combination with agents that act as DNA damage response modulators, e.g. PARP inhibitors, ATM inhibitors and ATR inhibitors.

In one aspect, the present invention relates to a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, for use in the treatment of a proliferative disorder.

In another aspect, the present invention relates to a use of a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administration with a DNA damage response modulator (e.g. a PARP inhibitor, an ATM inhibitor and/or an ATR inhibitor), or a pharmaceutically acceptable salt thereof.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administration with a DNA damage response modulator (e.g. a PARP inhibitor, an ATM inhibitor and/or an ATR inhibitor), or a pharmaceutically acceptable salt thereof.

In another aspect, the present invention relates to a method of treating a proliferative disorder comprising administering to a subject in need thereof a therapeutically effective amount of a compound as defined herein, or a pharmaceutically acceptable salt thereof, as defined herein and a DNA damage response modulator (e.g. a PARP inhibitor, an ATM inhibitor and/or an ATR inhibitor), or a pharmaceutically acceptable salt thereof, either sequentially, separately or simultaneously.

Any DNA damage response modulator (e.g. a PARP inhibitor, an ATM inhibitor and/or an ATR inhibitor) may be used in the combination therapy defined herein.

Combination Therapy with DNA Damage Response Modulators

The compounds of the present invention are particularly suited to use in combination with agents that act as DNA damage response modulators, e.g. PARP inhibitors, ATM inhibitors and ATR inhibitors.

In one aspect, the present invention relates to a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, for use in the treatment of a proliferative disorder.

In another aspect, the present invention relates to a use of a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, in the manufacture of a medicament for treating of a proliferative disorder.

In another aspect, the present invention relates to a method of treating of a proliferative disorder in a subject in need thereof comprising administering to said subject a combination comprising a compound as defined herein, or a pharmaceutically acceptable salt thereof, and a DNA damage response modulator (e.g. a PARP inhibitor, an ATM inhibitor and/or an ATR inhibitor), or a pharmaceutically acceptable salt thereof, as defined herein.

In another aspect, the present invention relates to a compound as defined herein, or a pharmaceutically acceptable salt thereof, as defined herein for use in the treatment of a proliferative disorder, wherein the compound, or a pharmaceutically acceptable salt thereof, is for simultaneous, separate or sequential administration with a DNA damage response modulator (e.g. a PARP inhibitor, an ATM inhibitor and/or an ATR inhibitor), or a pharmaceutically acceptable salt thereof.

In another aspect, the present invention relates to a use of a compound as defined herein, or a pharmaceutically acceptable salt thereof, as defined herein in the manufacture of a medicament for treating a proliferative disorder, wherein the medicament is for simultaneous, separate or sequential administration with a DNA damage response modulator (e.g. a PARP inhibitor, an ATM inhibitor and/or an ATR inhibitor), or a pharmaceutically acceptable salt thereof.

In another aspect, the present invention relates to a method of treating a proliferative disorder comprising administering to a subject in need thereof a therapeutically effective amount of a compound as defined herein, or a pharmaceutically acceptable salt thereof, as defined herein and a DNA damage response modulator (e.g. a PARP inhibitor, an ATM inhibitor and/or an ATR inhibitor), or a pharmaceutically acceptable salt thereof, either sequentially, separately or simultaneously.

Any DNA damage response modulator (e.g. a PARP inhibitor, an ATM inhibitor and/or an ATR inhibitor) may be used in the combination therapy defined herein.

### Probe Compounds of the Invention

In another aspect, the present invention provides a probe compound of Formula (I), or sub-formulae (I-I) to (I-XLV), or a salt thereof, as defined herein, wherein one of R¹² or R¹³ is a group -L-Q or -Lₓ-X as defined herein.

The probe compounds of the present invention are selective for MLH1.

The linker group L may be any suitable linker moiety that connects the detection moiety Q to the remainder of the probe compound of Formula (I), or sub-formulae (I-I) to (I-XLV), defined herein.

Suitably, the linker group L is 3 to 30 atoms in length, more suitably, 4 to 20 atoms in length and even more suitably 5 to 18 atoms in length. In a particular group of probe compounds of Formula (I), or sub-formulae (I-I) to (I-XLV), defined herein, L is 5 to 12 atoms in length.

Suitably, L is as defined in any one paragraphs (115) to (146) above. More suitably as defined in any one of numbered paragraphs (139) to (146) above. In a particular group of probe compounds of Formula (I), or sub-formulae (I-I) to (I-XLV), L is as defined in paragraph (144), (145) or (146) above.

The detection moiety Q can be any moiety that can enable the probe compound of formula (I) to be detected and quantified. As described further below, the probe compounds of formula (I) defined herein are designed to be used in displacement assays, whereby the ability of a test compound to displace the probe compound of formula (I) from the ATP-binding site of target protein (e.g. MLH1) can be used to determine the binding affinity of that test compound for the ATP-binding site of the target protein. Thus, the detection moiety Q can be any moiety that can be readily detected and quantified. In certain circumstances, the detection moiety Q enables any probe compounds of formula (I) that have been displaced (i.e. is “unbound”) from the ATP-binding site of the target protein to be detected and quantified. In certain embodiments of the invention, this may be achieved by collecting any displaced or “unbound” compound of formula (I) from the test sample and assaying the sample to determine how much unbound probe compound is present. This will in turn give an indication of how much of the probe compound present in the sample has been displaced by the test compound.

It will therefore be appreciated that the nature of detection moiety Q is not critical as long as it can be used to enable the amount of the probe compound of formula (I) present in a sample to be determined. A person skilled in the art will be able to select a suitable detection moiety Q and a suitable methodology for detecting and quantifying the amount of the compound of formula (I) in a sample, in particular to detect the amount of the probe compound of formula (I) that has been displaced from the ATP binding site of a target protein by a test compound.

Suitably, the detection moiety Q is selected from the group consisting of a fluorophore, an oligonucleotide, a biomolecule, a molecular sensor, a protein, or a peptide.

In embodiments where the detection moiety Q is an oligonucleotide, a biomolecule, a molecular sensor, a protein, or a peptide, then any suitable technique known in the art for detecting and quantifying the amount of the oligonucleotide, biomolecule, molecular sensor, protein, or peptide present may be utilised. For example, a fluorescently labelled secondary probe may be used that is capable of specifically binding to the detection moiety Q of the probe compound of formula (I) and, once any excess of the secondary probe has been removed, the amount of binding of the secondary probe to detection moiety Q of the compound for formula (I) can be detected and quantified, thereby enabling the amount of the probe compound of formula (I) to be determined.

For example, if Q is an oligonucleotide, then a secondary probe having a suitable detectable label, e.g. a fluorophore or radio-label, and a complimentary oligonucleotide sequence capable of hybridising to Q can be used to detect and quantify the amount of the probe compound of formula (I) present in sample (and suitably the amount of the probe compound displaced from the ATP-binding site of the target protein by a test compound). Similarly, if Q is a protein or peptide, then a secondary probe may be an antibody capable of selectively binding to that protein or peptide and a suitable detectable label, e.g. a fluorophore or radio-label.

More suitably, the detection moiety Q is a fluorophore. In such cases, the compound for formula (I) can be used in a fluorescence polarisation assay. In a particular group of probe compounds of formula (I), the detection moiety Q is a fluorophore selected from the group consisting of AlexaFluor dyes, Cyanine dyes, fluorescein, BODIPY or BODIPY derivatives (e.g. BODIPY TMR), TAMRA, Oregon Green dyes, FITC, Ru(bpy)3, Rhodamine dyes, Acridine orange, and Texas Red. In a further group of probe compounds of formula (I), the detection moiety Q is a fluorophore selected from the group consisting of AlexaFluor-647, AlexaFluor-633, AlexaFluor-594, AlexaFluor-488, Cyanine-5B, Cyanine-3B, Fluorescein, BODIPY TMR, TAMRA, Oregon Green 488, Oregon Green 514, FITC, Ru(bpy)3, Rhodamine dyes, Acridine orange, and Texas Red.

Suitably, Q is as defined in any one of numbered paragraphs (147) to (150) above. More suitably, Q is as defined in any one of numbered paragraphs (148) to (150) above. Most suitably, Q is as defined numbered paragraph (149) or (150) above.

It will be appreciated that X may be any suitable functional group that is capable of reacting with a functional group present on a detection moiety Q, or a compound of the formula Q-L₂-Y as defined herein, to covalently bind the detection moiety to the compound of formula (I) and thereby form a compound of formula (I) as defined herein.

A person skilled in the art will be familiar with appropriate functional groups that can be used to form covalent linkages with a suitable detection moiety. For example, X may be selected from halo, N₃ or ethynyl (—C≡CH).

In a particular embodiment, X is N₃ or ethynyl.

Suitably, X is a functional group that is capable of reacting with a functional group present on the detection moiety to form a bond or triazole linkage between the detection moiety and the compound of formula (I). In a particular embodiment, the linkage formed is a triazole linkage (which can be formed by CLICK chemistry).

Suitably, X is as defined in any one of numbered paragraphs (151) to (156) above. More suitably, X is as defined in any one of numbered paragraphs (153) to (156) above. Most suitably, X is as defined numbered paragraph (156) above.

In certain embodiments of the invention, a compound of Formula (I), or sub-formulae (I-I) to (I-XLV) in which one of R¹² or R¹³ is a group -L-Q is formed by reacting a compound of Formula (I), or sub-formulae (I-I) to (I-XLV) in which one of R¹² or R¹³ is a group -Lₓ-X with a group of the formula:

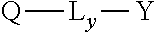

wherein:
Q is a detection moiety as defined hereinbefore:
L_{y} is absent or a linker group which, when covalently bound to the group Lₓ of the compound of formula I forms a linker L of the compound of Formula (I), or sub-formulae (I-I) to (I-XLV);
Y is a functional group capable of reacting with the functional group X present on the compound of Formula (I), or sub-formulae (I-I) to (I-XLV) to covalently link Q-L₂ to Lₓ of the compound of Formula (I), or sub-formulae (I-I) to (I-XLV) and form a group -L-Q.

In an embodiment, L_{y} is a (1-8C)alkylene linker, wherein the alkylene chain optionally further comprises one or more —O—, —C(O)NR_{q1}—, —NR_{q1}C(O)—, —C(O)O—, —OC(O)— or triazole ring linkages positioned either within the alkylene chain and/or at one of its termini, wherein R_{q1} is hydrogen or (1-2C)alkyl.

In another embodiment, L_{y} is a (1-8C)alkylene linker, wherein the alkylene chain optionally further comprises one —O—, —C(O)NR_{q1}—, —NR_{q1}C(O)—, —C(O)O—, —OC(O)— or triazole ring linkage positioned either within the alkylene chain and/or at one of its termini, wherein R_{q1} is hydrogen or (1-2C)alkyl.

In a further embodiment, L_{y} is a (1-5C)alkylene linker.

In another embodiment, L_{y} is a (1-3C)alkylene linker.

It will be appreciated that Y may be any suitable functional group that is capable of reacting with a functional group X present on a probe compound of formula I to covalently bind the group Q-L_{y} to the compound of formula I and thereby form a compound of formula I as defined herein.

A person skilled in the art will be familiar with appropriate functional groups that can be used to form covalent linkages with a functional group X on the compound of Formula (I), or sub-formulae (I-I) to (I-XLV). For example, Y may be selected from halo, N₃ or ethynyl.

In a particular embodiment, Y is N₃ when X is ethynyl or Y is ethynyl when X is N₃.

Suitably, Y is a functional group that is capable of reacting with a functional group X present on the probe compound of Formula (I), or sub-formulae (I-I) to (I-XLV) to form a bond or triazole linkage. In a particular embodiment, the linkage formed is a triazole linkage (which can be formed by CLICK chemistry).

The linker group Lₓ may be any suitable linker moiety that connects the functional group X to the remainder of the probe compound of Formula (I), or sub-formulae (I-I) to (I-XLV) defined herein. Suitably, Lₓ is as defined in any one of numbered paragraphs (157) to (161) above. More suitably, Lₓ is as defined in any one of numbered paragraphs (159) to (161) above. Most suitably, Lₓ is as defined numbered paragraph (161) above.

In a particular group of probe compounds of the present invention, the probe compound is a compound of Formula (I), or sub-formulae (I-I) to (I-XLV), wherein R², R³, R⁴, R⁵, Y₁, Y₂, A₁, A₂, A₃, A₄, R¹¹, R¹², R¹³ and R¹⁴ each have any one of the definitions provided hereinbefore, with the proviso that one of R¹² or R¹³ is a group:

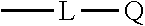

wherein L is a linker; and Q is a detection moiety.

Suitably, in the probe compounds of Formula (I), or sub-formulae (I-I) to (I-XLV), L is a linker as defined in any one of numbered paragraphs (139) to (146) above; and Q is a detection moiety as defined in any one of numbered paragraphs (148) to (150) above. More suitably, L is a linker as defined in any one of numbered paragraphs (144), (145) or (146) above; and Q is a detection moiety as defined in numbered paragraph (149) or (150) above.

In another group of probe compounds of the present invention, the compound is a compound of the Formula (I), or sub-formulae (I-I) to (I-XLV) shown above, or a pharmaceutically acceptable salt, hydrate and/or solvate thereof, wherein wherein R², R³, R⁴, R⁵, Y₁, Y₂, A₁, A₂, A₃, A₄, R¹¹, R¹², R¹³ and R¹⁴ each have any one of the definitions provided hereinbefore, with the proviso that one of R¹² or R¹³ is a group:

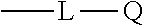

wherein L is a linker and Q is a detection moiety.

Suitably, in the probe compounds of Formula (I), or sub-formulae (I-I) to (I-XLV), L is a linker as defined in any one of numbered paragraphs (139) to (146) above; and Q is a detection moiety as defined in any one of numbered paragraphs (148) to (150) above. More suitably, L is a linker as defined in any one of numbered paragraphs (144), (145) or (146) above; and Q is a detection moiety as defined in numbered paragraph (149) or (150) above.

In a particular group of probe compounds of Formula (I), or sub-formulae (I-I) to (I-XLV) defined herein, R² is as defined in paragraph (1) above.

In a particular group of probe compounds of Formula (I), or sub-formulae (I-I) to (I-XLV) defined herein, R⁴ is as defined in numbered paragraph (5) above.

In a particular group of probe compounds of Formula (I), or sub-formulae (I-I) to (I-XLV) defined herein, R⁴ is as defined in numbered paragraph (6) above.

In a particular group of probe compounds of Formula (I), or sub-formulae (I-I) to (I-XLV) defined herein, R⁴ is as defined in numbered paragraph (7) above.

In a particular group of probe compounds of Formula (I), or sub-formulae (I-I) to (I-XLV) defined herein, R³ is as defined in numbered paragraph (12) above.

In a particular group of probe compounds of Formula (I), or sub-formulae (I-I) to (I-XLV) defined herein, R³ is as defined in numbered paragraph (13) above.

In a particular group of probe compounds of Formula (I), or sub-formulae (I-I) to (I-XLV) defined herein, R³ is as defined in numbered paragraph (14) above.

In a particular group of probe compounds of Formula (I), or sub-formulae (I-I) to (I-XLV) defined herein, R³ and R⁴ are as defined in numbered paragraph (18) above.

In a particular group of probe compounds of Formula (I), or sub-formulae (I-I) to (I-XLV) defined herein, R³ and R⁴ are as defined in numbered paragraph (19) above.

In a particular group of probe compounds of Formula (I), or sub-formulae (I-I) to (I-XLV) defined herein, R³ and R⁴ are as defined in numbered paragraph (20) above.

In a particular group of probe compounds of Formula (I), or sub-formulae (I-I) to (I-XLV) defined herein, R³ and R⁴ are as defined in numbered paragraph (21) above.

In a particular group of probe compounds of Formula (I), or sub-formulae (I-I) to (I-XLV) defined herein, R⁵ is as defined in numbered paragraph (23) above.

In a particular group of probe compounds of Formula (I), or sub-formulae (I-I) to (I-XLV) defined herein, R⁵ is as defined in numbered paragraph (24) above.

In a particular group of probe compounds of Formula (I), or sub-formulae (I-I) to (I-XLV) defined herein, R⁶ is as defined in numbered paragraph (34) above.

In a particular group of probe compounds of Formula (I), or sub-formulae (I-I) to (I-XLV) defined herein, R⁶ is as defined in numbered paragraph (35) above.

In a particular group of probe compounds of Formula (I), or sub-formulae (I-I) to (I-XLV) defined herein, R⁶ is as defined in numbered paragraph (36) above.

In a particular group of probe compounds of Formula (I), or sub-formulae (I-I) to (I-XLV) defined herein, A₁, A₂, A₃ and A₄ are as defined in numbered paragraph (64) above.

In a particular group of probe compounds of Formula (I), or sub-formulae (I-I) to (I-XLV) defined herein, R¹¹ is as defined in numbered paragraph (84) above.

In a particular group of probe compounds of Formula (I), or sub-formulae (I-I) to (I-XLV) defined herein, R¹¹ is as defined in numbered paragraph (85) above.

In a particular group of probe compounds of Formula (I), or sub-formulae (I-I) to (I-XLV) defined herein, R¹¹ is as defined in numbered paragraph (86) above.

In a particular group of probe compounds of Formula (I), or sub-formulae (I-I) to (I-XLV) defined herein, R¹² is as defined in numbered paragraph (94) above.

In a particular group of probe compounds of Formula (I), or sub-formulae (I-I) to (I-XLV) defined herein, R¹² is as defined in numbered paragraph (95) above.

In a particular group of probe compounds of Formula (I), or sub-formulae (I-I) to (I-XLV) defined herein, R¹² is as defined in numbered paragraph (96) above.

In a particular group of probe compounds of Formula (I), or sub-formulae (I-I) to (I-XLV) defined herein, R¹³ is as defined in numbered paragraph (99) above.

In a particular group of probe compounds of Formula (I), or sub-formulae (I-I) to (I-XLV) defined herein, R¹³ is as defined in numbered paragraph (100) above.

In a particular group of probe compounds of Formula (I), or sub-formulae (I-I) to (I-XLV) defined herein, R¹³ is as defined in numbered paragraph (101) above.

In a particular group of probe compounds of Formula (I), or sub-formulae (I-I) to (I-XLV) defined herein, R¹⁴ is as defined in numbered paragraph (103) above.

In a particular group of probe compounds of Formula (I), or sub-formulae (I-I) to (I-XLV) defined herein, R¹⁴ is as defined in numbered paragraph (104) above.

In a particular group of probe compounds of Formula (I), or sub-formulae (I-I) to (I-XLV) defined herein, R¹⁴ is as defined in numbered paragraph (105) above.

In a particular group of probe compounds of Formula (I), or sub-formulae (I-I) to (I-XLV) defined herein, R^{a} is as defined in numbered paragraph (112) above.

In a particular group of probe compounds of Formula (I), or sub-formulae (I-I) to (I-XLV) defined herein, R^{a} is as defined in numbered paragraph (113) above.

In a particular group of probe compounds of Formula (I), or sub-formulae (I-I) to (I-XLV) defined herein, R^{a} is as defined in numbered paragraph (114) above.

A suitable salt of a probe compound of the invention is, for example, an acid-addition salt of a compound of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable salt of a compound of the invention which is sufficiently acidic is an alkali metal salt, for example a sodium or potassium salt, an alkaline earth metal salt, for example a calcium or magnesium salt, an ammonium salt or a salt with an organic base.

The present invention also encompasses probe compounds of the invention as defined herein which comprise one or more isotopic substitutions. For example, H may be in any isotopic form, including ¹H, ²H (D) and ³H (T); C may be in any isotopic form, including ¹²C, ¹³C and ¹⁴C; and O may be in any isotopic form, including ¹⁶O and ¹⁸O; and the like.

It is also to be understood that certain probe compounds of the formula (I) may exist in solvated as well as unsolvated forms such as, for example, hydrated forms.

It is also to be understood that certain probe compounds of the formula (I) may exhibit polymorphism, and that the invention encompasses all such polymorphic forms.

Certain probe compounds of formula (I) may also exist in a number of different tautomeric forms and references to compounds of the formula (I) include all such forms.

### Synthesis of the Probe Compounds

In another aspect, the present invention provides a method of synthesising a probe compound of Formula (I), or sub-formulae (I-I) to (I-XLV), or a salt thereof, as defined herein.

The probe compounds of the present invention can be prepared by any suitable technique known in the art. Particular processes for the preparation of these compounds are described further in the accompanying examples.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green; 4^{th} Edition; 2006 (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

In one aspect, the present invention provides a process for synthesising a compound of formula (I) defined herein, the process comprising reacting a compound of Formula (I), or sub-formulae (I-I) to (I-XLV), in which one of R¹² or R¹³ is a group —Lₓ-X as defined herein, with a compound of the formula Q-L₂-Y as defined herein to form a compound of Formula (I), or sub-formulae (I-I) to (I-XLV); and optionally thereafter:
(i) removing any protecting groups present;
(ii) forming a salt of the compound of Formula (I), or sub-formulae (I-I) to (I-XLV); and/or
(iii) isolating and purifying the compound of Formula (I), or sub-formulae (I-I) to (I-XLV).

### Applications of the Probe Compounds

In one aspect, the present invention provides the use of a probe compound of Formula (I), or sub-formulae (I-I) to (I-XLV), or a salt thereof, as defined herein in a displacement assay to determine the binding affinity of a test molecule for the ATP-binding site of a target protein.

In another aspect, the present invention provides a probe compound of Formula (I), or sub-formulae (I-I) to (I-XLV), or a salt thereof, for use in a displacement assay to determine the binding affinity of a test molecule for the ATP-binding site of a target protein.

In an embodiment, the target protein is MLH1.

Displacement assays are well known in the art. In embodiments of the invention in which Q is a fluorophore, the displacement assay is a fluorescence polarisation assay. Fluorescence polarisation assays are well known in the art.

The test compound may be any biologic or small molecule compound that is to be screened for binding to the ATP-binding site of the target protein (e.g. MLH1).

In another aspect, the present invention provides an assay for determining the binding affinity of a test molecule for the ATP-binding site of a target protein, the assay comprising:
(i) incubating a test molecule with the target protein in the presence of a probe compound of Formula (I), or sub-formulae (I-I) to (I-XLV), or a salt thereof, as defined herein; and
(ii) determining whether any probe compound is displaced from the ATP-binding site of the target protein.

In an embodiment, the target protein is MLH1.

Suitably, the assay is a displacement assay. Displacement assays are well known in the art. In embodiments of the invention in which Q is a fluorophore, the displacement assay is a fluorescence polarisation assay. Fluorescence polarisation assays are well known in the art.

The step of determining whether any probe compound is displaced from the ATP-binding site of the target protein suitably comprises detecting whether the test compound results in an increase in the proportion of probe compound of Formula (I), or sub-formulae (I-I) to (I-XLV) that is unbound to the ATP-binding site. This can be achieved by either detecting whether there is an increase in the amount of unbound probe compound (relative to a control) or, if the amount of bound probe compound can be readily detected (e.g. by fluorescence polarisation) then detecting whether there is a decrease in the amount of bound probe compound (again relative to a control).

The determination of the proportion of the probe compound that is bound to the ATP-binding site of the target protein relative to the amount that is unbound can be achieved by techniques known in the art. The particular techniques utilised will depend on the nature of the detection moiety Q in the compound of Formula (I), or sub-formulae (I-I) to (I-XLV). Suitably, Q is a fluorophore and the assay is a fluorescence polarisation assay. In such assays, the difference in the fluorescence caused by the displacement of probe compound from the ATP-binding site of the target protein can be detected and used to quantify the amount of any displaced probe compound—and thereby determine the binding affinity of the test compound for the ATP binding site of the target protein.

In another aspect, the present invention provides a method for determining the binding affinity of a test molecule for the ATP-binding site of a target protein, the assay comprising:
(i) incubating a test molecule with the target protein in the presence of a probe compound of Formula (I), or sub-formulae (I-I) to (I-XLV), or a salt thereof, as defined herein; and
(ii) determining whether any probe compound is displaced from the ATP-binding site of the target protein.

In an embodiment, the target protein is MLH1.

Suitably, the method is a displacement assay. Displacement assays are well known in the art. In embodiments of the invention in which Q is a fluorophore, the displacement assay is a fluorescence polarisation assay. Fluorescence polarisation assays are well known in the art.

The step of determining whether any probe compound is displaced from the ATP-binding site of the target protein can be conducted in the manner described above.

In another aspect, the present invention provides an assay for determining the location and/or quantity of a target protein present within a biological sample, the assay comprising:
(i) contacting the biological sample with a probe compound of Formula (I), or sub-formulae (I-I) to (I-XLV), or a salt thereof, as defined herein; and
(ii) determining location and/or quantity of the compound of Formula (I), or sub-formulae (I-I) to (I-XLV) present within the sample by detecting the location and/or intensity of the detection moieties of the compounds of Formula (I), or sub-formulae (I-I) to (I-XLV) present within the biological sample.

In another aspect, the present invention provides an assay for determining the location and/or quantity of a target protein present within a biological sample, the assay comprising:
(i) contacting the biological sample with a probe compound of Formula (I), or sub-formulae (I-I) to (I-XLV) in which one of R¹² or R¹³ is a group -Lₓ-X as defined herein, or a salt thereof, as defined herein; and
(ii) contacting the biological sample with a detection moiety Q, or a compound of the formula Q-L₂-Y as defined herein, that is capable of reacting with the functional group X present on the compound of Formula (I), or sub-formulae (I-I) to (I-XLV) to form a compound of Formula (I), or sub-formulae (I-I) to (I-XLV) in which one of R¹² or R¹³ is a group -L-Q as defined herein in situ within the biological sample;
(iii) determining location and/or quantity of the compound of Formula (I), or sub-formulae (I-I) to (I-XLV) present within the sample by detecting the location and/or intensity of the detection moieties of the compounds of Formula (I), or sub-formulae (I-I) to (I-XLV) present within the biological sample.

In another aspect, the present invention provides a method for determining the location and/or quantity of a target protein present within a biological sample, the method comprising:
(i) contacting the biological sample with a probe compound of Formula (I), or sub-formulae (I-I) to (I-XLV), or a salt thereof, as defined herein; and
(ii) determining location and/or quantity of the compound of Formula (I), or sub-formulae (I-I) to (I-XLV) present within the sample by detecting the location and/or intensity of the detection moieties of the compounds of Formula (I), or sub-formulae (I-I) to (I-XLV) present within the biological sample.

In another aspect, the present invention provides a method for determining the location and/or quantity of a target protein present within a biological sample, the method comprising:
(i) contacting the biological sample with a detection moiety Q, or a compound of the formula Q-L₂-Y as defined herein, that is capable of reacting with the functional group X present on the compound of Formula (I), or sub-formulae (I-I) to (I-XLV) in which one of R¹² or R¹³ is a group —Lₓ-X as defined herein, to form a compound of Formula (I), or sub-formulae (I-I) to (I-XLV) in which one of R¹² or R¹³ is a group -L-Q as defined herein in situ within the biological sample;
(ii) determining location and/or quantity of the compound of Formula (I), or sub-formulae (I-I) to (I-XLV) present within the sample by detecting the location and/or intensity of the detection moieties of the compounds of Formula (I), or sub-formulae (I-I) to (I-XLV) present within the biological sample.

In an embodiment, the target protein is MLH1.

Suitably the step of contacting the biological sample with a probe compound of Formula (I), or sub-formulae (I-I) to (I-XLV) (step (i) in the above assays and methods), involves incubating the biological sample with the probe compound under conditions that enable it to bind to the ATP-binding site of a target protein (e.g. MLH1).

The determination of the distribution and quantity of the compound of formula (I) within the biological sample can be achieved by techniques known in the art. The particular techniques utilised will depend on the nature of the detection moiety Q in the compound of formula (I). Suitably, Q is a fluorophore and the determination of location and quantity of the compound of formula (I) within the sample can be achieved by fluorescence microscopy/imaging. Appropriate calibration of the fluorescence intensity can be used to determine the quantity of the compound of formula (I) at various locations within the sample.

The assays and methods of the invention defined herein may optionally further comprise one or more washing steps conducted after step (i) to remove excess probe compound of Formula (I), or sub-formulae (I-I) to (I-XLV) from the biological sample.

### EXAMPLES

### Abbreviations

ACN for acetonitrile
Boc for tert-butyloxycarbonyl
CPME for cyclopentyl methyl ether
DAST for diethylaminosulfur trifluoride
DCM for dichloromethane
DIPEA for N,N-diisopropylethylamine, Hünig's base
DMAP for 4-(dimethylamino) pyridine
DME for dimethyl ether
DMF for N,N-dimethylformamide
DMSO for dimethylsulfoxide
DPPF for 1,1′-Bis(diphenylphosphino)ferrocene
EDC for 1-ethyl-3-(3-dimethylaminopropyl)carbodiimide
EtOAc for ethyl acetate
FA for formic acid
h for hours
HATU for (1-[bis(dimethylamino)methylene]-1H-1,2,3-triazolo[4,5-b]pyridinium 3-oxide
hexafluorophosphate N
HOBT for N-hydroxybenzotriazole
HPLC for High Pressure Liquid Chromatography
IPA for isopropyl alcohol
LAH for Lithium aluminium hydride
LCMS for Liquid Chromatography-Mass Spectrometry
MI for Molecular Ion
min for minutes
MW for microwave
NMM for N-methylmorpholine
NMR for Nuclear Magnetic Resonance
PdCl₂(PPh₃)₂ for Bis(triphenylphosphine)palladium chloride
Pd(dppf)₂Cl₂ for [1,1′-bis(diphenylphosphino)ferrocene]dichloropalladium(II)
(Pd(dba)₂) for bis(dibenzylideneacetone)palladium
RT for room temperature
SFC for supercritical fluid chromatography
TFAA for trifluoroacetic anhydride
TFA for trifluoroacetic acid
THF for tetrahydrofuran

### Analytical Methods

Commercially available starting materials, reagents and dry solvents were used as supplied. Flash chromatography or glass column chromatography was performed using Merck silica gel 230-400 mesh size or Interchim PuriFlash HC silica columns. Flash chromatography was also performed on combi-flash RF Teledyne Isco or Biotage Isolera machines. Preparative TLC was performed on Merck plates.

¹H Nuclear magnetic resonance (NMR) spectroscopy was carried out using a Bruker instrument operating at 400 MHz using the stated solvent at around room temperature unless otherwise stated. In all cases, NMR data were consistent with the proposed structures. Characteristic chemical shifts (5) are given in parts-per-million using conventional abbreviations for designation of major peaks: e.g. s, singlet; d, doublet; t, triplet; q, quartet; dd, doublet of doublets; dt, doublet of triplets; m, multiplet; br, broad.

### Liquid Chromatography-Mass Spectrometry Methods

### Method-A

Waters Acquity UPLC with binary solvent manager, PDA detector and Acquity QDA performance mass detector, column: X-Bridge BEH C18, 50×2.1 mm, 2.5 micron, column temperature: 35° C., auto sampler temperature: 5° C., mobile phase A: 0.1% (v/v) formic acid in water (pH=2.70), Mobile Phase B: 0.1% formic acid (v/v) in water:acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 mins. Mass detector parameter: ionization mode was cycled through positive and negative modes with cone voltage 10 V and 30 V and 0.8 kV capillary voltage, temperature of source and probe were 120° C. and 600° C. respectively.

### Method-B

Waters Acquity with PDA detector and SQ Detector, column: X-Bridge BEH C18, 50×2.1 mm, 2.5 micron, column temperature: 35° C., auto sampler temperature: 5° C., mobile phase A: 5 mM ammonium bicarbonate in water (pH=7.35), mobile phase B: acetonitrile; mobile phase gradient details: t=0 min (97% A, 3% B) flow: 0.5 mL/min; t=0.2 min (97% A, 3% B) flow: 0.5 mL/min; gradient to t=2.7 min (2% A, 98% B) flow: 0.5 mL/min; gradient to t=3 min (0% A, 100% B) flow: 0.7 mL/min; t=3.5 min (0% A, 100% B) flow: 0.7 mL/min; gradient to t=3.51 min (97% A, 3% B) flow: 0.5 mL/min; end of run at t=4 min (97% A, 3% B), flow rate: 0.5 mL/min, analysis time 4 mins. Mass detection parameter: ionization mode was cycled through positive and negative mode with cone voltage 10 V and 30 V and 3.25 kV capillary voltage, temperature of source and probe were 120° C. and 400° C. respectively.

### Method-C

Waters Acquity UPLC with binary solvent manager, PDA detector and Acquity QDA performance mass detector, column: YMC Tri-art C18, 50×2 mm, 1.9 micron, column temperature: 35° C., auto sampler temperature: 5° C., mobile phase A: 0.1% (v/v) formic acid in water (pH=2.70), Mobile Phase B: 0.1% formic acid (v/v) in water:acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 mins. Mass detector parameter: ionization mode was cycled through positive and negative modes with cone voltage 10 V and 30 V and 0.8 kV capillary voltage, temperature of source and probe were 120° C. and 600° C. respectively.

### Method-CA

Waters Acquity UPLC-MS with Waters DAD and Waters SQD2, column: Acquity UPLC HSS C18 1.8 um 100×2.1 mm (plus guard cartridge), mobile phase A: Water (High purity via PureLab Option unit) with 0.1% formic acid, mobile phase B: Acetonitrile (Far UV grade) with 0.1% (V/V) formic acid, flow: 0.4 ml/min, mobile phase gradient details: t=0 min (95% A, 5% B); t=0.4 min (95% A, 5% B); gradient to t=6.0 min (5% A, 95% B); t=6.8 min (5% A, 95% B); gradient to t=7 min (95% A, 5% B); end of run at t=8 min (95% A, 5% B), sample: 0.5-2 ul (concentration ~0.2-1 mg/ml), DAD detector parameters: UV, diode array 210 nm-400 nm, Resolution 1.2 nm, mass detector parameters: MS, mass 100-700 in ES+ & ES− (300 μl/min split to MS).

### Method-D

Waters Acquity UPLC with quaternary solvent manager, with PDA detector and SQ detector, column: X-Bridge BEH C18, 50*2.1 mm, 2.5 micron, column temperature: 35° C., auto sampler temperature: 5° C., mobile phase A: 0.1% (v/v) Formic acid in water (pH=2.70), mobile phase B: 0.1% (v/v) formic acid in water:acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 mins. Mass detector parameter: ESI capillary probe, ionization mode cycled through positive and negative modes with cone voltage 10 V and 30V and 0.8 kV capillary voltage, temperature of source and probe were 120° C. and 400° C. respectively.

### Method-E

Waters Acquity UPLC with binary solvent manager, PDA detector and Acquity QDA performance mass detector, column: Welch Xtimate C18, 50*2.1 mm, 1.8 micron, column temperature: 35° C., auto sampler temperature: 5° C., mobile phase A: 0.1% (v/v) formic acid in water (pH=2.70), Mobile Phase B: 0.1% formic acid (v/v) in water:acetonitrile (10:90), mobile phase gradient details: t=0 min (97% A, 3% B) flow: 0.8 mL/min; t=0.75 min (97% A, 3% B) flow: 0.8 mL/min; gradient to t=2.7 min (2% A, 98% B) flow: 0.8 mL/min; gradient to t=3 min (0% A, 100% B) flow: 1 mL/min; t=3.5 min (0% A, 100% B) flow: 1 mL/min; gradient to t=3.51 min (97% A, 3% B) flow: 0.8 mL/min; end of run at t=4 min (97% A, 3% B), Flow rate: 0.8 mL/min, analysis time 4 mins. Mass detector parameter: ionization mode was cycled through positive and negative modes with cone voltage 10 V and 30 V and 0.8 kV capillary voltage, temperature of source and probe were 120° C. and 600° C. respectively.

### Method-F

Waters Acquity UPLC with PDA detector and SQ Detector, column: Welch-Xtimate, C18 4.6*50 mm, 5 micron, column temperature: 35° C., auto sampler temperature: 5° C., mobile phase A: 5 mM ammonium bicarbonate in water (pH=7.35), mobile phase B: acetonitrile; mobile phase gradient details: t=0 min (97% A, 3% B) flow: 0.5 mL/min; t=0.2 min (97% A, 3% B) flow: 0.5 mL/min; gradient to t=2.7 min (2% A, 98% B) flow: 0.5 mL/min; gradient to t=3 min (0% A, 100% B) flow: 0.7 mL/min; t=3.5 min (0% A, 100% B) flow: 0.7 mL/min; gradient to t=3.51 min (97% A, 3% B) flow: 0.5 mL/min; end of run at t=4 min (97% A, 3% B), flow rate: 0.5 mL/min, analysis time 4 min. Mass detection parameter: ionization mode was cycled through positive and negative mode with cone voltage 10 V and 30 V and 3.25 kV capillary voltage, temperature of source and probe were 120° C. and 400° C. respectively.

### Method-G

Waters Acquity UPLC with binary solvent manager, PDA detector and Acquity QDA performance mass detector, column: X-Bridge C18 2.1*50 mm3.5 micron, column temperature: 35° C., auto sampler temperature: 5° C., mobile phase A: 5 mM ammonium bicarbonate in water (pH=7.35), mobile phase B: acetonitrile; mobile phase gradient details: t=0 min (97% A, 3% B) flow: 0.5 mL/min; t=0.2 min (97% A, 3% B) flow: 0.5 mL/min; gradient to t=2.7 min (2% A, 98% B) flow: 0.5 mL/min; gradient to t=3 min (0% A, 100% B) flow: 0.7 mL/min; t=3.5 min (0% A, 100% B) flow: 0.7 mL/min; gradient to t=3.51 min (97% A, 3% B) flow: 0.5 mL/min; end of run at t=4 min (97% A, 3% B), flow rate: 0.5 mL/min, analysis time 4 mins. Mass detector parameter: ionization mode was cycled through positive and negative modes with cone voltage 10 V and 30 V and 0.8 kV capillary voltage, temperature of source and probe were 120° C. and 600° C. respectively.

### Method-H

Waters 996 Photodiode Array Detector equipped with Waters Micromass ZQ detector, column: XTIMATE C18 5 μm 4.6*150 mm, Column temperature: 35° C., Auto sampler temperature: 15° C., Mobile Phase A: 5 mM Ammonium Acetate and 0.1% Formic acid (pH=3.50) in Milli Q water, Mobile Phase B: Methanol. Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—PDA. Mass parameter: Probe: ESI, Mode of Ionisation: Positive and Negative, Cone voltage:—30 and 10 V, capillary voltage:—3.0 KV, Extractor Voltage:—2 V, Rf Lens:—0.1 V, Temperature of source:—120° C., Temperature of Probe:—400° C., Cone Gas Flow:—100 L/Hr, Desolvation Gas flow:—800 L/Hr.

### Method-I

Waters Acquity UPLC-H Class equipped with PDA and attached with QDa detector, column: X-Bridge BEH C18, 50×2.1 mm, 2.5 micron, Column temperature: 30° C., Auto sampler temperature: 15° C., Mobile Phase A: 0.1% Formic acid in Milli Q water (pH=2.70), Mobile Phase B: 0.1% Formic acid in Milli Q water:Acetonitrile (10:90), Mobile phase gradient details: T=0 min (97% A, 3% B) flow: 0.8 mL/min; T=0.75 min (97% A, 3% B) flow: 0.8 mL/min; gradient to T=2.7 min (2% A, 98% B) flow: 0.8 mL/min; gradient to T=3 min (0% A, 100% B) flow: 1 mL/min; T=3.5 min (0% A, 100% B) flow: 1 mL/min; gradient to T=3.51 min (97% A, 3% B) flow: 0.8 mL/min; end of run at T=4 min (97% A, 3% B), Flow rate: 0.8 mL/min, Run Time:—4 min. UV Detection Method:—PDA Mass parameter: Probe:—ESI, Mode of Ionisation:—positive and negative, Cone voltage:—10V and 30V, capillary voltage:—0.8 KV, Extractor Voltage:—1 KV, Rf Lens:—0.1, Temperature of source:—120° C., Temperature of Probe:—600° C., Cone Gas Flow:—Default, Desolvation Gas flow:—Default.

### Method-J

Waters 996 Photodiode Array Detector equipped with Waters Micromass ZQ detector, column: XTIMATE C18 5 μm 4.6*150 mm, Column temperature: 60° C., Auto sampler temperature: 15° C., Mobile Phase A: 5 mM Ammonium Acetate and 0.1% Formic acid (pH=3.50) in Milli Q water, Mobile Phase B: Methanol. Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—PDA. Mass parameter: Probe: ESI, Mode of Ionisation: Positive and Negative, Cone voltage:—30 and 10 V, capillary voltage:—3.0 KV, Extractor Voltage:—2 V, Rf Lens:—0.1 V, Temperature of source:—120° C., Temperature of Probe:—400° C., Cone Gas Flow:—100 L/Hr, Desolvation Gas flow:—800 L/Hr.

### Method-K

Agilent 1260 Infinity-II DAD Detector equipped with MASS detector Agilent G6125C (LC/MSD), column: XTIMATE C18 5 μm 4.6*150 mm, Column temperature: 35° C., Auto sampler temperature: 15° C., Mobile Phase A: 5 mM Ammonium Acetate and 0.1% Formic acid (pH=3.50) in Milli Q water, Mobile Phase B: Methanol. Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—DAD. Mass parameter: Probe: MMI, Mode of Ionisation: (ESI) Positive and Negative, Fragment voltage:—30 and 70 V, capillary voltage:—3000 V, Gas temperature of source:—325° C., Temperature of Vaporizer:—225° C., Gas Flow:—12 L/min, Nebulizer:—50.

### Method-L

Agilent 1260 Infinity-II DAD Detector equipped with MASS detector Agilent G6125C (LC/MSD), column: XTIMATE C18 5 μm 4.6*150 mm, Column temperature: 60° C., Auto sampler temperature: 15° C., Mobile Phase A: 5 mM Ammonium Acetate and 0.1% Formic acid (pH=3.50) in Milli Q water, Mobile Phase B: Methanol. Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—DAD. Mass parameter: Probe: MMI, Mode of Ionisation: (ESI) Positive and Negative, Fragment voltage:—30 and 70 V, capillary voltage:—3000 V, Gas temperature of source:—325° C., Temperature of Vaporizer:—225° C., Gas Flow:—12 L/min, Nebulizer:—50.

### Method-M

Waters 996 Photodiode Array Detector equipped with Waters Micromass ZQ detector, column: XTIMATE C18 5 μm 4.6*150 mm, Column temperature: 35° C., Auto sampler temperature: 15° C., Mobile Phase A: 0.05% Trifluoroacetic acid (pH=3.50) in Milli Q water, Mobile Phase B: Acetonitrile. Mobile phase gradient details: T=0 min (100% A, 00% B); T=7.0 min (50% A, 50% B); gradient to T=9.0 min (00% A, 100% B); gradient to T=14.00 min (0% A, 100% B); T=14.01 min (100% A, 00% B); end of run at T=17 min (100% A, 00% B), Flow rate:—1.0 mL/min, Run Time:—17 min, UV Detection Method:—PDA. Mass parameter: Probe: ESI, Mode of Ionisation: Positive and Negative, Cone voltage:—30 and 10 V, capillary voltage:—3.0 KV, Extractor Voltage:—2 V, Rf Lens:—0.1 V, Temperature of source:—120° C., Temperature of Probe:—400° C., Cone Gas Flow:—100 L/Hr, Desolvation Gas flow:—800 L/Hr.

### Method-N

Waters Acquity UPLC-H Class equipped with PDA and attached with QDa detector, column: X-Bridge BEH C18, 50×2.1 mm, 2.5 micron, Column temperature: Ambient, Auto sampler temperature: 15° C., Mobile Phase A: 2 mM ammonium acetate followed by 0.1% Formic acid in water, Mobile Phase B: 0.1% Formic Acid in Acetonitrile, Mobile phase gradient details: T=0 min (95% A, 5% B) flow; T=0.4 min (95% A, 5% B); gradient to T=0.8 min (65% A, 35% B); gradient to T=1.20 min (45% A, 55% B); T=2.5 min (0% A, 100% B); gradient to T=3.30 min (0% A, 100% B); gradient to T=3.31 min to end of run at T=4 min (95% A, 5% B), Flow rate: 0.55 mL/min, Run Time: 4 min. UV Detection Method: PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default.

### Method-O

Waters Acquity UPLC-H Class equipped with PDA and attached with QDa detector, column: X-Bridge BEH C18, 50×2.1 mm, 2.5 micron, Column temperature: Ambient, Auto sampler temperature: 15° C., Mobile Phase A: 5 mM Ammonium Bicarbonate in Milli Q water, Mobile Phase B: Acetonitrile, Mobile phase gradient details: T=0 min (95% A, 5% B) flow; T=0.4 min (95% A, 5% B); gradient to T=0.8 min (65% A, 35% B); gradient to T=1.20 min (45% A, 55% B); T=2.5 min (0% A, 100% B); gradient to T=3.30 min (0% A, 100% B); gradient to T=3.31 min to end of run at T=4 min (95% A, 5% B), Flow rate: 0.55 mL/min, Run Time:—4 min. UV Detection Method: PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default.

### Method-P

Waters Acquity UPLC-H Class equipped with PDA and attached with QDa detector, column: Welch Xtimate C18, 150*4.6 mm, 5 micron, Column temperature: Ambient, Auto sampler temperature: 15° C., Mobile Phase A: 2 mM ammonium acetate followed by 0.1% Formic acid in water, Mobile Phase B: 0.1% Formic acid in Acetonitrile, Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.00 mL/min, Run Time: 17 min. UV Detection Method:—PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default

### Method-Q

Waters Acquity UPLC-H Class equipped with PDA and attached with QDa detector, column: Welch Xtimate C18, 150*4.6 mm, 5 micron, Column temperature: Ambient, Auto sampler temperature: 15° C., Mobile Phase A: 2 mM ammonium acetate followed by 0.1% Formic acid in water, Mobile Phase B: 0.1% Formic acid in Acetonitrile, Mobile phase gradient details: T=0 min (100% A, 0% B); T=7.0 min (50% A, 50% B); gradient to T=9.0 min (0% A, 100% B); gradient to T=14.00 min (0% A, 100% B); T=14.01 min (100% A, 0% B); end of run at T=17 min (100% A, 0% B), Flow rate: 1.00 mL/min, Run Time: 17 min. UV Detection Method:—PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default

### Method-R

Waters Acquity UPLC-H Class equipped with PDA and attached with QDa detector, column: Welch Xtimate C18, 150*4.6 mm, 5 micron, Column temperature: Ambient, Auto sampler temperature: 15° C., Mobile Phase A: 2 mM ammonium acetate followed by 0.1% Formic acid in water, Mobile Phase B: 0.1% Formic acid in Acetonitrile, Mobile phase gradient details: T=0 min (50% A, 50% B); T=7.0 min (10% A, 90% B); gradient to T=9.0 min (0% A, 100% B); gradient to T=14.00 min (0% A, 100% B); T=14.01 min (50% A, 50% B); end of run at T=17 min (50% A, 50% B), Flow rate: 1.00 mL/min, Run Time: 17 min. UV Detection Method:—PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default.

### Method-S

Waters Acquity UPLC-H Class equipped with PDA and attached with QDa detector, column: Welch Xtimate C18, 150*4.6 mm, 5 micron, Column temperature: Ambient, Auto sampler temperature: 15° C., Mobile Phase A: 0.05% Trifluoro Acetic Acid in Milli Q water, Mobile Phase B: Acetonitrile, Mobile phase gradient details: T=0 min (100% A, 0% B); T=7.0 min (50% A, 50% B); gradient to T=9.0 min (0% A, 100% B); gradient to T=14.00 min (0% A, 100% B); T=14.01 min (100% A, 0% B); end of run at T=17 min (100% A, 0% B), Flow rate: 1.00 mL/min, Run Time: 17 min. UV Detection Method:—PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default.

### Method-T

Waters Acquity UPLC with binary solvent manager, PDA detector and Acquity QDa detector, column: X-Bridge BEH C18, 50×2.1 mm, 2.5 micron, Column temperature: Ambient, Auto sampler temperature: 15° C., Mobile Phase A: 2 mM ammonium acetate followed by 0.1% Formic acid in water, Mobile Phase B: 0.1% Formic Acid in Acetonitrile, Mobile phase gradient details: T=0 min (95% A, 5% B) flow; T=0.4 min (95% A, 5% B); gradient to T=0.8 min (65% A, 35% B); gradient to T=1.20 min (45% A, 55% B); T=2.5 min (0% A, 100% B); gradient to T=3.30 min (0% A, 100% B); gradient to T=3.31 min to end of run at T=4 min (95% A, 5% B), Flow rate: 0.55 mL/min, Run Time: 4 min. UV Detection Method: PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default.

### Method-U

Waters Acquity UPLC-H Class equipped with PDA and attached with QDa detector, column: Welch Xtimate C18, 150*4.6 mm, 5 micron, Column temperature: 60° C. Auto sampler temperature: 15° C., Phase A: 5 mM Ammonium Bicarbonate in Milli Q water, Mobile Phase B: Acetonitrile, Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.00 mL/min, Run Time: 17 min. UV Detection Method:—PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default.

### Method-V

Agilent 1260 Infinity-II DAD Detector equipped with MASS detector Agilent G6125C (LC/MSD), column: XTIMATE C18 5 μm 4.6*150 mm, Column temperature: 60° C., Auto sampler temperature: 15° C., Mobile Phase A: 5 mM Ammonium Bicarbonate in Milli Q water, Mobile Phase B: Acetonitrile. Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—DAD. Mass parameter: Probe: MMI, Mode of Ionisation: (ESI) Positive and Negative, Fragment voltage:—30 and 70 V, capillary voltage:—3000 V, Gas temperature of source:—325° C., Temperature of Vaporizer:—225° C., Gas Flow:—12 L/min, Nebulizer:—50

### Method-W

Waters Acquity UPLC-H Class equipped with PDA and attached with QDa detector, column: Welch Xtimate C18, 150*4.6 mm, 5 micron, Column temperature: Ambient Auto sampler temperature: 15° C., Phase A: 5 mM Ammonium Bicarbonate in Milli Q water, Mobile Phase B: Acetonitrile, Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.00 mL/min, Run Time: 17 min. UV Detection Method:—PDA Mass parameter: Probe: ESI, Mode of Ionisation: positive and negative, Cone voltage: 10V and 30V, capillary voltage: 0.8 KV, Extractor Voltage: 1 KV, Rf Lens: 0.1, Temperature of source: 120° C., Temperature of Probe: 600° C., Cone Gas Flow:—Default, Desolvation Gas flow:—Default.

### Method-X

Waters 996 Photodiode Array Detector equipped with Waters Micromass ZQ detector, column: XTIMATE C18 5 μm 4.6*150 mm, Column temperature: 60° C., Auto sampler temperature: 15° C., Mobile Phase A: 0.05% Trifluoro acetic acid (pH=3.50) in Milli Q water, Mobile Phase B: Acetonitrile. Mobile phase gradient details: T=0 min (100% A, 00% B); T=7.0 min (50% A, 50% B); gradient to T=9.0 min (00% A, 100% B); gradient to T=14.00 min (0% A, 100% B); T=14.01 min (100% A, 00% B); end of run at T=17 min (100% A, 00% B), Flow rate:—1.0 mL/min, Run Time:—17 min, UV Detection Method:—PDA. Mass parameter: Probe: ESI, Mode of Ionisation: Positive and Negative, Cone voltage:—30 and 10 V, capillary voltage:—3.0 KV, Extractor Voltage:—2 V, Rf Lens:—0.1 V, Temperature of source:—120° C., Temperature of Probe:—400° C., Cone Gas Flow:—100 L/Hr, Desolvation Gas flow:—800 L/Hr.

### Method-Z

Waters Acquity UPLC-H Class equipped with PDA and attached with SQd detector, column: Welch Xtimate C18, 150*4.6 mm, 5 micron, Column temperature: Ambient, Auto sampler temperature: 15° C., Mobile Phase A: 2 mM ammonium acetate followed by 0.1% Formic acid in water, Mobile Phase B: 0.1% Formic acid in Acetonitrile, Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate: 1.00 mL/min, Run Time: 17 min. UV Detection Method:—PDA Mass parameter: Probe: ESI, Mode of Ionisation: positive and negative, Cone voltage: 10V and 30V, capillary voltage: 3.0 KV, Extractor Voltage: 1 KV, Rf Lens: 0.1, Temperature of source: 120° C., Temperature of Probe: 600° C., Cone Gas Flow:—Default, Desolvation Gas flow:—Default

### LC-Method 1 (Acidic FA)

UPLC-MS was performed on a Waters DAD+Waters SQD2, single quadrupole UPLC-MS spectrometer using an Acquity UPLC HSS Shield RP18 1.7 um 100×2.1 mm (Plus guard cartridge), maintained at 40° C. column being initially held at 5% Acetonitrile (Far UV grade) with 0.1% (V/V) formic acid/Water (High purity via PureLab Option unit) with 0.1% formic acid for 0.4 minutes, followed by a linear gradient of 5-95% within 6.4 minutes and then held at 95% for 1.2 minutes (F=0.4 mL/min).

### LC-Method 2 (Basic FA)

UPLC-MS was performed on a Waters DAD+Waters SQD2, single quadrupole UPLC-MS spectrometer using an Acquity UPLC BEH Shield RP18 1.7 um 100×2.1 mm (Plus guard cartridge), maintained at 40° C. column being initially held at 5% acetonitrile/water (with 10 mM ammonium bicarbonate) for 0.4 minutes, followed by a linear gradient of 5-95% within 6.4 minutes and then held at 95% for 1.2 minutes (F=0.4 mL/min).

### Analytical HPLC Methods

### Method A

Machine Details:—Agilent 1100 Series with PDA detector, Column temperature: 25° C., Auto sampler temperature: 25° C., Mobile Phase A: 0.05% Trifluoroacetic acid in Milli Q water (pH=2.1), Mobile Phase B: Acetonitrile (100%).

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—PDA.

### Method B

Machine Details:—Agilent 1260 Series with PDA detector, Column temperature: 25° C., Auto sampler temperature: 25° C., Mobile Phase A: 0.05% Trifluoroacetic acid in Milli Q water (pH=2.1), Mobile Phase B: Acetonitrile (100%).

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—PDA.

### Method C

Machine Details:—Water alliance e2695 with 2998 PDA detector, Column temperature: 25° C., Auto sampler temperature: 25° C., Mobile Phase A: 0.1% ammonium hydroxide solution in HPLC water Mobile Phase B: Acetonitrile (100%).

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—PDA.

### Method D

Machine Details:—Shimadzu i-series LC-2050C with PDA detector, Column temperature: 25° C., Auto sampler temperature: 25° C., Mobile Phase A: 0.05% Trifluoroacetic acid in Milli Q water (pH=2.1), Mobile Phase B: Acetonitrile (100%).

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—PDA.

### Method E

Machine Details:—Agilent 1260 Infinity-II Series with DAD detector, Column temperature: 25° C., Auto sampler temperature: 25° C., Mobile Phase A: 0.05% Trifluoroacetic acid in Milli Q water (pH=2.1), Mobile Phase B: Acetonitrile (100%).

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—DAD.

### Method F

Machine Details:—Agilent 1260 Infinity-II Series with DAD detector, Column temperature: 35° C., Auto sampler temperature: 25° C., Mobile Phase A: 5 mM Ammonium Acetate and 0.1% Formic acid (pH=3.50) in Milli Q water, Mobile Phase B: Methanol (100%).

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—DAD.

### Method G

Machine Details:—Waters 996 Photodiode Array Detector, Column temperature: 60° C., Auto sampler temperature: 25° C., Mobile Phase A: 5 mM Ammonium Acetate and 0.1% Formic acid (pH=3.50) in Milli Q water, Mobile Phase B: Methanol (100%).

Mobile phase gradient details: T=0 min (90% A, 10% B); T=7.0 min (10% A, 90% B); gradient to T=9.0 min (0% A, 100% B); gradient to T=14.00 min (0% A, 100% B); T=14.01 min (90% A, 10% B); end of run at T=17 min (90% A, 10% B), Flow rate:—1.0 mL/min, Run Time:—17 min, UV Detection Method:—PDA.

### Method H

Machine Details:—Shimadzu LC-2050-C with PDA detector (HP-09), Water alliance e2695 with 2998 PDA detector (HP-11) and Agilent Infinity II G6125C (HP-05), Column temperature: 25° C., Auto sampler temperature: 25° C., Mobile Phase A: 0.1% FA in Water in Milli Q water, Mobile Phase B: Acetonitrile (100%).

Mobile phase gradient details: T=0 min (90% A, 10% B) T=7 min (10% A, 90% B) T=9 min (0% A, 100% B) T=14 min (0% A, 100% B) T=14.01 min (90% A, 10% B) gradient to T=17 min (90% A, 10% B). Flow rate:—1.0 ml/min, Run Time:—17 min, UV Detection Method:—PDA.

### Method I

Machine Details:—Water alliance e2695 with 2998 PDA detector (HP-04), Agilent Infinity II G6125C (HP-05), Column temperature: 25° C., Auto sampler temperature: 25° C., Mobile Phase A: 5 mM Ammonium Bicarbonate in Water in Milli Q water, Mobile Phase B: Acetonitrile (100%).

Mobile phase gradient details: T=0 min (90% A, 10% B) T=7 min (10% A, 90% B) T=9 min (0% A, 100% B) T=14 min (0% A, 100% B) T=14.01 min (90% A, 10% B) gradient to T=17 min (90% A, 10% B), Flow rate:—1.0 ml/min, Run Time:—17 min, UV Detection Method:—PDA.

### Method J

Machine Details:—Shimadzu LC-2050-C with PDA detector (HP-09), Water alliance e2695 with 2998 PDA detector (HP-11) and Agilent Infinity II G6125C (HP-05), Column temperature: 25° C., Auto sampler temperature: 25° C., Mobile Phase A: 0.1% FA in Water in Milli Q water, Mobile Phase B: Acetonitrile (100%).

Mobile phase gradient details: T=0 min (100% A, 0% B) T=7 min (50% A, 50% B) T=9 min (0% A, 100% B) T=14 min (0% A, 100% B) T=14.01 min (100% A, 0% B) gradient to T=17 min (100% A, 0% B). Flow rate:—1.0 ml/min, Run Time:—17 min, UV Detection Method:—PDA.

### Method K

Machine Details:—Water alliance e2695 with 2998 PDA detector (HP-04), Agilent Infinity II G6125C (HP-05), Column temperature: 25° C., Auto sampler temperature: 25° C., Mobile Phase A: 5 mM Ammonium Bicarbonate in Water in Milli Q water, Mobile Phase B: Acetonitrile (100%).

Mobile phase gradient details: T=0 min (100% A, 0% B) T=7 min (50% A, 50% B) T=9 min (0% A, 100% B) T=14 min (0% A, 100% B) T=14.01 min (100% A, 0% B) gradient to T=17 min (100% A, 0% B). Flow rate:—1.0 ml/min, Run Time:—17 min, UV Detection Method:—PDA.

### Method L

Machine Details:—Water Acquity UPLC-H Class equipped with PDA, Column temperature: 60° C., Auto sampler temperature: 15° C., Mobile Phase A: 5 mM Ammonium Bicarbonate in Water in Milli Q water, Mobile Phase B: Acetonitrile (100%).

Mobile phase gradient details: T=0 min (90% A, 10% B) T=7 min (10% A, 90% B) T=9 min (0% A, 100% B) T=14 min (0% A, 100% B) T=14.01 min (90% A, 10% B) gradient to T=17 min (90% A, 10% B). Flow rate:—1.0 ml/min, Run Time:—17 min, UV Detection Method:—PDA.

### Method M

Machine Details:—Agilent 1260 Infinity-II DAD Detector equipped, Column temperature: 60° C., Auto sampler temperature: 15° C., Mobile Phase A: 5 mM Ammonium Bicarbonate in Water in Milli Q water, Mobile Phase B: Acetonitrile (100%).

Mobile phase gradient details: T=0 min (90% A, 10% B) T=7 min (10% A, 90% B) T=9 min (0% A, 100% B) T=14 min (0% A, 100% B) T=14.01 min (90% A, 10% B) gradient to T=17 min (90% A, 10% B). Flow rate:—1.0 ml/min, Run Time:—17 min, UV Detection Method:—PDA.

### Method: N

Machine Details:—Shimadzu LC-2050-C with PDA detector (HP-09), Water alliance e2695 with 2998 PDA detector (HP-11) and Agilent Infinity II G6125C (HP-05), Column temperature: 35° C., Auto sampler temperature: 25° C., Mobile Phase A: 0.05% Trifluoro acetic acid in Milli Q water (pH=2.1), Mobile Phase B: Acetonitrile (100%).

### Purification Methods

### Preparative Reverse-Phase HPLC Conditions

Preparative HPLC purification was performed by reverse phase HPLC using a Waters Fractionlynx preparative HPLC system (2525 pump, 2996/2998 UV/VIS detector, 2767 liquid handler) or an equivalent HPLC system such as a Gilson Trilution UV directed system. The Waters 2767 liquid handler acted as both auto-sampler and fraction collector. The columns used for the preparative purification of the compounds were a Waters Sunfire OBD Phenomenex Luna Phenyl Hexyl or Waters Xbridge Phenyl at 10 μm 19×150 mm or Waters CSH Phenyl Hexyl, 19×150, 5 μm column unless otherwise stated. Appropriate focused gradients were selected based on acetonitrile and methanol solvent systems under either acidic or basic conditions. The modifiers used under acidic/basic conditions were formic acid or TFA (0.1% V/V) and ammonium bicarbonate (10 mM) respectively. The purification was controlled by Waters Fractionlynx software through monitoring at 210-400 nm and triggered a threshold collection value at 260 nm and, when using the Fractionlynx, the presence of target molecular ion as observed under API conditions. Collected fractions were analysed by LCMS (Waters Acquity systems with Waters SQD).

Below is a list of methods and conditions used for preparative reverse phase HPLC purifications.

| Prep HPLC | |
|---|---|
| method | Conditions |
| prep-LC-2 | Luna Phenyl-Hexyl 21.2 × 150 mm, 10 um |
| | 20-80% MeOH/H₂O (0.1% FA), 20 ml/min, RT |
| prep-LC-3 | Sunfire C18 19 × 15 0 mm, 10 um 5-60% |
| | ACN/H₂O (0.1% FA), 20 ml/min, RT |
| prep-LC-5 | Xbridge Phenyl 19 × 150 mm, 10 um 20-80% |
| | MeOH/H₂O (10 mM NH₄CO₃), 20 ml/min, RT |
| prep-LC-9 | Sunfire C18 19 × 150 mm, 10 μm 20-80% |
| | ACN/H₂O (0.1% FA), 20 ml/min, RT |
| prep-LC-12 | Xbridge Phenyl 19 × 150 mm, 10 um 40-100% |
| | MeOH/H₂O (1 mM NH₄CO₃), 20 ml/min, RT |
| prep-LC-13 | Xbridge C18 19 × 150 mm, 10 um 20-80% ACN/H₂O |
| | (10 mM NH₄CO₃), 20 ml/min, RT |
| prep-LC-14 | Xbridge C18 3 × 50 mm, 3 um 5-95% ACN/H₂O |
| | (10 mM NH₄CO₃), 1.7 ml/min, RT |
| Prep-LC-15 | Luna Phenyl-Hexyl 21.2 × 150 mm, 10 um |
| | 40-100% MeOH/H₂O (0.1% FA), 20 ml/min, RT |

### Chiral Supercritical Fluid Chromatography (SFC) Separation Protocol

The enantiomeric separation of compounds was achieved by Supercritical Fluid Chromatography (SFC) using a Waters Thar Prep100 preparative SFC system (P200 CO₂ pump, 2545 modifier pump, 2998 UV/VIS detector, 2767 liquid handler with Stacked Injection Module). The Waters 2767 liquid handler acted as both auto-sampler and fraction collector. Appropriate isocratic methods were selected based on methanol, ethanol or isopropanol solvent systems under un-modified or basic conditions. The standard SFC method used was modifier, CO₂, 100 mL/min, 120 Bar backpressure, 40° C. column temperature. The modifier used under basic conditions was diethylamine (0.1% V/V). The modifier used under acidic conditions was either formic acid (0.1% V/V) or TFA (0.1% V/V). The SFC purification was controlled by Waters Fractionlynx software through monitoring at 210-400 nm and triggered at a threshold collection value, typically 260 nm. Collected fractions were analysed by SFC (Waters/Thar SFC systems with Waters SQD). The fractions that contained the desired product were concentrated by vacuum centrifugation.

Below is a list of SFC methods and conditions used to resolve enantiomers or to determine enantiomeric purity.

| SFC | |
|---|---|
| method | Conditions for chiral resolution |
| SFC-1 | WATERS VIRIDIS 2-EP 20 × 250 mm, 5 um 15-25% |
| | MeOH (0.1% NH₄OH)/CO₂, 100 |
| | ml/min, 120 bar, 40° C., DAD 250 nm |
| SFC-2 | WATERS VIRIDIS 2-EP 20 × 250 mm, 5 um 10-20% |
| | MeOH (0.1% NH₄OH)/CO₂, 100 |
| | ml/min, 120 bar, 40° C., DAD 250 nm |
| SFC-4 | REPROSPHER PEI 100 20 × 150 mm, 5 um 30-40% |
| | MeOH (0.1% NH₄OH)/CO₂, 100 |
| | ml/min, 120 bar, 40° C., DAD 220 nm |
| SFC-5 | LUX Cellulose-IC5 20 × 250 mm, 5 um 55/45 |
| | EtOH (0.1% NH₄OH)/CO₂, 100 ml/min, |
| | 120 bar, 40° C., DAD 260 nm |
| SFC-6 | LUX Cellulose-IC5 20 × 250 mm, 5 um 55/45 |
| | EtOH (0.1% NH₄OH)/CO₂, 100 ml/min, |
| | 120 bar, 40° C., DAD 260 nm |

### Preparation of Compounds

### Example 1: (S)-(2-(Benzyloxy)-6-hydroxy-4-methoxyphenyl)(4-((tetrahydrofuran-3-yl)amino)isoindolin-2-yl)methanone

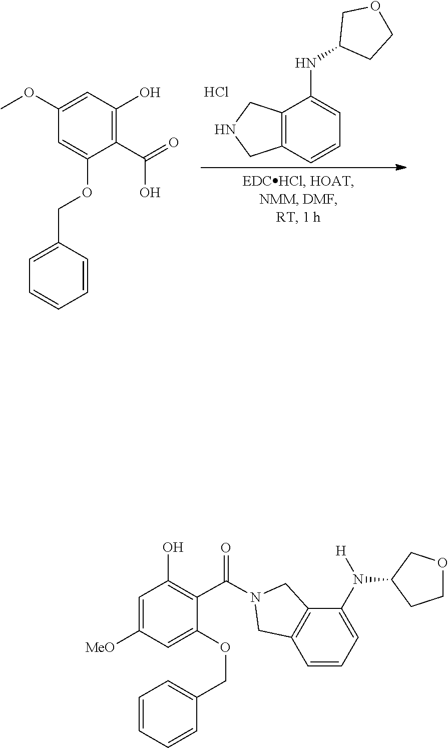

To a stirred solution of 2-(benzyloxy)-6-hydroxy-4-methoxybenzoic acid (Intermediate C) (0.2 g, 0.72 mmol, 1 eq.) in DMF (2 mL) was added EDC·HCl (0.210 g, 1.09 mmol, 1.5 eq.) followed by HOAT (0.1 g, 0.72 mmol, 1 eq.) and the reaction mixture was stirred at room temperature for 30 mins. (S)—N-(Tetrahydrofuran-3-yl)isoindolin-4-amine hydrochloride (Intermediate A) (0.174 g, 0.72 mmol, 1.0 eq.) and NMM (0.4 mL, 3.64 mmol, 5 eq.) were added and stirring continued at room temperature for 1 h. The mixture was poured into cold water (20 mL) and the resulting precipitate was collected by filtration and dried under vacuum. Purification of the crude material by flash chromatography eluting with 50% EtOAc in hexane yielded the title compound as an off-white solid (0.05 g, Yield: 14.0%). ¹H NMR (DMSO-d6, 400 MHz): δ 1.76-1.87 (m, 1H), 2.09-2.20 (m, 1H), 3.49-3.60 (m, 1H), 3.65-3.76 (m, 4H), 3.82-3.04 (m, 3H), 4.37-4.40 (m, 2H), 4.58-4.67 (m, 2H), 5.10 (s, 2H), 5.34-5.46 (m, 1H, D₂O exchangeable), 6.11-6.21 (m, 2H), 6.43-6.62 (m, 2H), 7.05-7.13 (m, 1H), 7.24-7.33 (m, 5H), 9.78 (d, J=8.8 Hz, 1H).

LCMS (Method A): 1.929 min, MS: ES+ 461.1 (M+1).

HPLC (Method D): 7.877 min.

### Example 2: (R)-(2-(Benzyloxy)-6-hydroxy-4-methoxyphenyl)(4-((tetrahydrofuran-3-yl)amino)isoindolin-2-yl)methanone

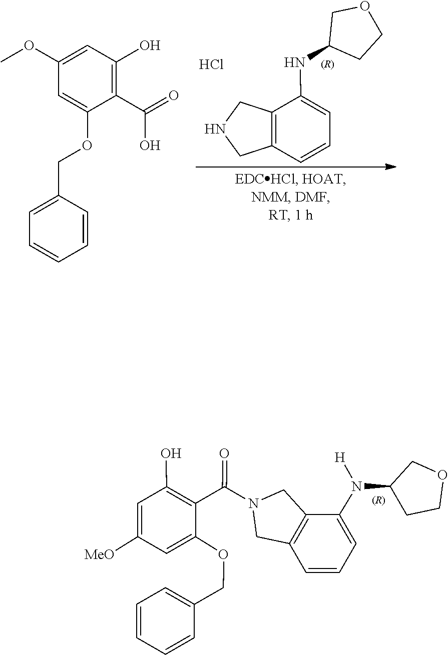

To a stirred solution of 2-(benzyloxy)-6-hydroxy-4-methoxybenzoic acid (Intermediate C) (0.2 g, 0.72 mmol, 1 eq.) in DMF (2 mL) was added EDC·HCl (0.210 g, 1.09 mmol, 1.5 eq.) followed by HOAT (0.1 g, 0.72 mmol, 1 eq.) and the reaction mixture was stirred at room temperature for 30 mins. (R)—N-(Tetrahydrofuran-3-yl)isoindolin-4-amine hydrochloride (Intermediate B) (0.174 g, 0.72 mmol, 1.0 eq.) and NMM (0.4 mL, 3.64 mmol, 5 eq.) were added and stirring continued at room temperature for 1 h. The mixture was poured into cold water (50 mL) and the resulting precipitate collected by filtration and dried under vacuum. Purification of the crude material by flash chromatography eluting with 50% EtOAc in hexane yielded the title compound as an off-white solid (0.015 g, Yield: 5.0%)

¹H NMR (DMSO-d6, 400 MHz): δ 1.76-1.87 (m, 1H), 2.09-2.20 (m, 1H), 3.36-3.60 (m, 1H), 3.65-3.76 (m, 4H), 3.82-3.04 (m, 3H), 4.37-4.40 (m, 2H), 4.58-4.67 (m, 2H), 5.10 (s, 2H), 5.34-5.47 (m, 1H, D₂O exchangeable), 6.12-6.22 (m, 2H), 6.43-6.62 (m, 2H), 7.05-7.13 (m, 1H), 7.24-7.33 (m, 5H), 9.78 (d, J=8.8 Hz, 1H).

LCMS (Method A): 1.931 min, MS: ES+ 461 (M+1).

HPLC (Method D): 7.893 min.

### Example 3: (R)-(2-(Benzyloxy)-4-(difluoromethyl)-6-hydroxyphenyl)(4-((tetrahydrofuran-3-yl)amino)isoindolin-2-yl)methanone

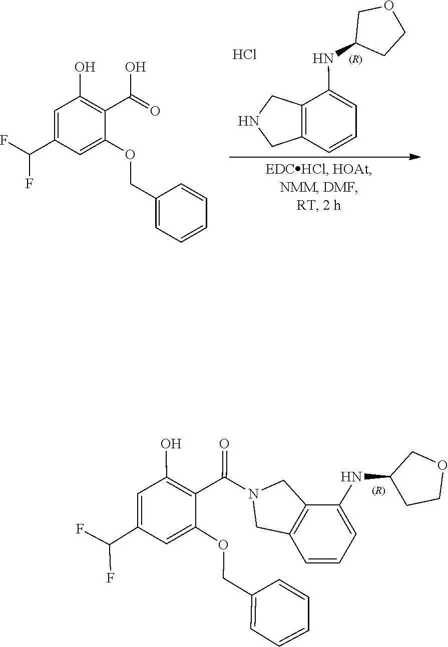

To a stirred solution 2-(benzyloxy)-4-(difluoromethyl)-6-hydroxybenzoic acid (Intermediate D) (0.25 g, 0.85 mmol, 1.0 eq.) in DMF (2.5 mL) was added EDC·HCl (0.245 g, 0.12 mmol, 1.2 eq.) followed by HOAT (0.115 g, 0.85 mmol, 1.0 eq.), NMM (0.430 g, 4.20 mmol, 5.0 eq.) and (R)—N-(tetrahydrofuran-3-yl)isoindolin-4-amine hydrochloride (Intermediate B) (0.245 g, 0.10 mmol, 1.2 eq.). The reaction mixture was stirred at room temperature for 2 h. The resulting mixture was poured into water (100 mL) and extracted with EtOAc (3×100 mL). The combined organic extracts were washed with cold brine solution (3×100 mL), dried over Na₂SO₄, filtered and concentrated under vacuum. The crude material was purified by flash chromatography followed by preparative TLC to yield the title compound (0.160 g, Yield: 15.0%).

High Temperature ¹H NMR (DMSO-d6, 400 MHz): δ 1.80-1.95 (m, 1H), 2.13-2.20 (m, 1H) 3.50-3.53 (m, 1H), 3.60-3.62 (m, 1H), 3.67-3.69 (m, 1H), 3.85-3.89 (m, 1H), 3.94-3.96 (m, 1H), 4.00-4.03 (m, 1H), 4.39-4.42 (m, 2H), 4.65-4.73 (m, 2H), 4.82 (bs, 1H), 5.14-5.23 (m, 3H) 6.48-6.50 (m, 2H) 6.76-6.79 (m, 1H), 6.84-6.90 (m, 1H), 7.04-7.14 (m, 1H), 7.14-7.35 (m 4H), 9.83 (bs, 1H).

LCMS (Method J): 8.499 min, MS: ES+ 481 (M+1).

HPLC (Method G): 8.499 min.

### Example 4: (S)-(2-(Benzyloxy)-4-(difluoromethyl)-6-hydroxyphenyl)(4-((tetrahydrofuran-3-yl)amino)isoindolin-2-yl)methanone

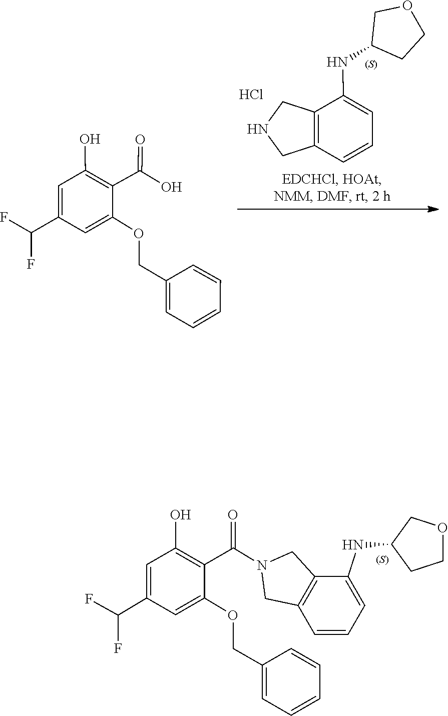

To a stirred solution 2-(benzyloxy)-4-(difluoromethyl)-6-hydroxybenzoic acid (Intermediate D) (0.25 g, 0.85 mmol, 1.0 eq.) in DMF (2.5 mL) were added EDC·HCl (0.245 g, 0.12 mmol, 1.2 eq.), HOAT (0.115 g, 0.85 mmol, 1.0 eq.), NMM (0.430 g, 4.20 mmol, 5.0 eq.) and (S)—N-(tetrahydrofuran-3-yl)isoindolin-4-amine hydrochloride (Intermediate A) (0.245 g, 0.10 mmol, 1.2 eq.) at room temperature. The reaction mixture was stirred at room temperature for 2 h. The resulting mixture was poured into ice cold water (100 mL) and extracted with EtOAc (3×100 mL). The combined organic extracts were washed with cold brine solution (3×100 mL), dried over Na₂SO₄, filtered and concentrated under vacuum. The crude material was purified by preparative TLC (5% MeOH in CM, NH₃ system) to yield the title compound (0.162 g, Yield: 27.6%).

High Temperature ¹H NMR (DMSO-d6, 400 MHz): δ 2.13-2.20 (m, 1H) 3.50-3.53 (m, 1H), 3.60-3.62 (m, 1H), 3.67-3.69 (m, 1H), 3.85-3.89 (m, 1H), 3.94-3.96 (m, 1H), 4.00-4.03 (m, 1H), 4.39-4.42 (m, 2H), 4.65-4.73 (m, 2H), 4.82 (bs, 1H), 5.14-5.23 (m, 2H), 6.48-6.50 (m, 2H), 6.62-6.91 (m, 3H), 7.05-7.14 (m, 1H), 7.24-7.35 (m, 5H), 9.69 (bs, 1H).

LMS (Method J): 8.499 min, MS: ES+ 481.56 (M+1).

HPLC (Method G): 8.505 min.

The compounds of the following tabulated Examples (Table Ex4) were prepared analogously to Example 4 from the indicated intermediates.

TABLE Ex4

| Ex. | Structure and Name/Ref | Analytical Data |
|---|---|---|
| 4.1 | 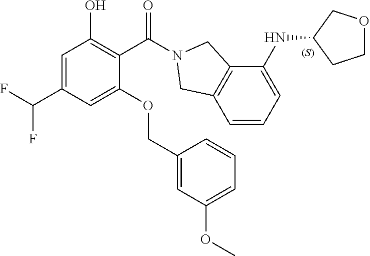 | HT ¹H NMR (DMSO-d6, 400 MHz, 349K): δ 1.70-1.90 (m, 1H), 2.08- 2.19 (m, 1H), 3.48-3.21 (m, 1H), 3.59 (d, J = 9.6 Hz, 3H), 3.66-3.70 (m, 1H), 3.71-3.80 (m, 1H), 3.81- 3.89 (m, 1H), 3.91-3.95 (m, 1H), 3.97-4.05 (m, 2H), 4.40 (d, J = 15.6 Hz, 2H), 4.63 (s, 1H), 4.71-4.72 (m, 1H), 5.15-5.16 (m, 2H), 5.37-5.50 (m, 1H), 6.43-6.49 (m, 1H), 6.62 (d, J = 7.6 Hz, 1H), 6.75-6.76 (m, 1H), 6.79-6.84 (m, 2H), 6.89-6.96 (m, 3H), 7.05-7.14 (m, 1H), 7.17-7.22 (m, 1H), 10.27 (s, 1H). LCMS (Method P): 8.481 min, MS: ES+ 511.2 (M + 1). HPLC (Method C): 5.70 min |
| | (S)-(4-(Difluoromethyl)-2-hydroxy-6-((3- | |
| | methoxybenzyl)oxy)phenyl)(4- | |
| | ((tetrahydrofuran-3-yl)amino)isoindolin-2- | |
| | yl)methanone | |
| | Prepared from 4-(difluoromethyl)-2-hydroxy- | |
| | 6-((3-methoxybenzyl)oxy)benzoic acid | |
| | (Intermediate DA) and (S)-N- | |
| | (tetrahydrofuran-3-yl)isoindolin-4-amine | |
| | hydrochloride (Intermediate A) | |
| 4.2 | 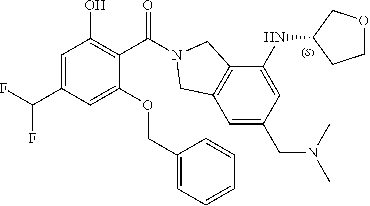 | HT ¹H NMR (DMSO-d6, 400 MHz, 349K): δ 1.77-1.70 (m, 1H), 1.88- 1.89 (m, 1H), 2.12-2.19 (m, 1H), 2.22 (s, 3H), 2.26 (s, 3H), 3.42 (s, 1H), 3.47 (s, 1H), 3.49-3.99 (m, 7H), 4.38 (d, J = 13.2 Hz, 2H), 4.63 (s, 1H), 4.71 (s, 1H), 5.16 (d, J = 2 Hz, 2H), 6.43-6.60 (m, 2H), 6.76- 7.04 (m, 3H), 7.22-7.29 (m, 3H), 7.33-7.35 (m, 2H), 10.0 (s, 1H). LCMS (Method A): 1.357 min, MS ES+ : 538.2 (M + 1). |
| | (S)-(2-(Benzyloxy)-4-(difluoromethyl)-6- | |
| | hydroxyphenyl)(6-((dimethylamino)methyl)- | |
| | 4-((tetrahydrofuran-3-yl)amino)isoindolin-2- | |
| | yl)methanone | |
| | Prepared from 2-(benzyloxy)-4- | |
| | (difluoromethyl)-6-hydroxybenzoic acid | |
| | (Intermediate D) and (S)-6- | |
| | ((dimethylamino)methyl)-N-(tetrahydrofuran- | |
| | 3-yl)isoindolin-4-amine hydrochloride | |
| | (Intermediate ZB). | |
| 4.3 | 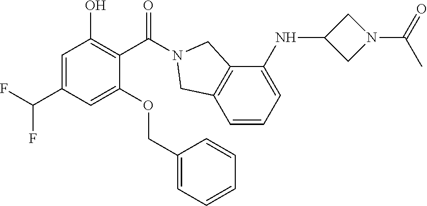 [SMILES: CC(=O)N1CC(Nc2cccc3c2CN(C(=O)c2c(O)cc(C(F)F)cc2OCc2ccccc2)C3)C1] | ¹H NMR (DMSO-d6, 400 MHz): δ 1.72-1.78 (m, 3H), 3.63 (s, 1H), 3.72-3.76 (m, 1H), 3.93-3.95 (m, 1H), 4.08-4.51 (m, 5H), 4.52-4.86 (m, 2H), 5.17 (d, J = 4 Hz, 2H), 5.88 (d, J = 5.6 Hz, 1H), 6.26-6.31 (m, 1H), 6.54 (d, J = 7.2 Hz, 1H), 6.75 (d, J = 4.8 Hz, 1H), 6.82 (t, J = 3.2 Hz, 1H), 7.06-7.14 (m, 1H), 7.23-7.34 (m, 5H), 10.32 (s, 1H). ¹H NMR is complex due to rotamers. LCMS (Method F): 2.29 min, MS: ES+ 508.4 (M + 1). |
| | 1-(3-((2-(2-(Benzyloxy)-4-(difluoromethyl)-6- | |
| | hydroxybenzoyl)isoindolin-4- | |
| | yl)amino)azetidin-1-yl)ethan-1-one | |
| | Prepared from 2-(benzyloxy)-4- | |
| | (difluoromethyl)-6-hydroxybenzoic acid | |
| | (Intermediate D) and 1-(3-(isoindolin-4- | |
| | ylamino)azetidin-1-yl)ethan-1-one TFA salt | |
| | (Intermediate N). | |
| 4.4 | 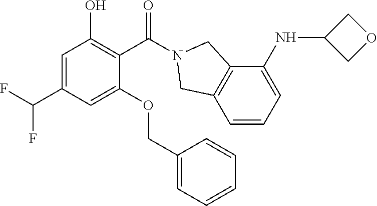 [SMILES: O=C(c1c(O)cc(C(F)F)cc1OCc1ccccc1)N1Cc2cccc(NC3COC3)c2C1] | HT ¹H NMR (DMSO-d6, 400 MHz, 348K): δ 4.43-4.44 (m, 3H), 4.47- 4.63 (m, 2H), 4.69 (s, 2H), 4.74- 4.85 (m, 2H), 5.17 (d, J = 4.8 Hz, 2H), 5.76-5.90 (dd, J = 5.6 Hz, 5.2 Hz, 1H, D₂O exchangeable), 6.18- 6.24 (m, 1H), 6.51-6.67 (dd, J = 7.6 Hz, 1H), 6.77 (m, 1H), 6.85 (d, J = 5.6 Hz, 1H), 6.91-7.11 (m, 2H), 7.24-7.34 (m, 5H), 9.98 (bs, 1H). LCMS (Method F): 2.46 min, MS: ES+ 467.4 (M + 1). |
| | (2-(Benzyloxy)-4-(difluoromethyl)-6- | |
| | hydroxyphenyl)(4-(oxetan-3- | |
| | ylamino)isoindolin-2-yl)methanone | |
| | Prepared from 2-(benzyloxy)-4- | |
| | (difluoromethyl)-6-hydroxybenzoic acid | |
| | (Intermediate D) and N-(oxetan-3- | |
| | yl)isoindolin-4-amine TFA salt (Intermediate | |
| | NB) | |
| 4.5 | 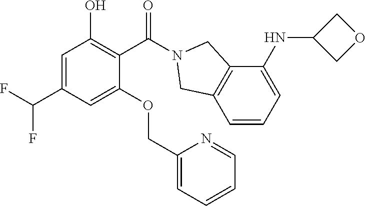 [SMILES: O=C(c1c(O)cc(C(F)F)cc1OCc1ccccn1)N1Cc2cccc(NC3COC3)c2C1] | HT ¹H NMR (DMSO-d6, 400 MHz, 349K): δ 4.63-4.41 (m, 5H), 4.71 (s, 1H), 4.76-4.71 (m, 2H), 4.78- 4.80 (m, 1H), 5.23 (d, J = 4.8 Hz, 2H), 5.90-6.11 (m, 1H, D₂O exchangeable), 6.15-6.21 (m, 1H), 6.52-6.67 (m, 1H), 6.78-6.95 (m, 3H), 7.07-7.11 (m, 1H), 7.27 (dd, J = 6.8 Hz, J = 12.0 Hz, 1H), 7.39 (d, J = 7.2 Hz, 1H), 7.69 (t, J = 7.2 Hz, 1H), 8.50 (t, J = 4.8 Hz, 1H), 9.99 (s, 1H). LCMS (Method X): 4.550 min, MS: ES+ 468.3 (M + 1). |
| | (4-(Difluoro methyl)-2-hydroxy-6-(pyridin-2- | HPLC (Method N): 4.70 min. |
| | ylmethoxy) phenyl) (4-(oxetan-3-ylamino) | |
| | isoindolin-2-yl) methanone | |
| | Prepared from 4-(difluoromethyl)-2-hydroxy- | |
| | 6-(pyridin-2-ylmethoxy)benzoic acid | |
| | (Intermediate DB) and N-(oxetan-3- | |
| | yl)isoindolin-4-amine TFA salt (Intermediate | |
| | NB) | |
| 4.6 | 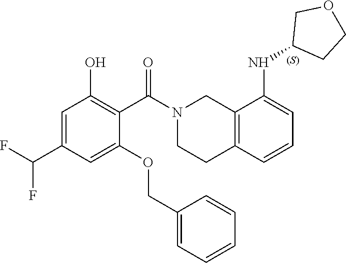 | HT ¹H NMR (DMSO-d6, 400 MHz, 349K): δ 1.90-1.92 (m, 1H), 2.19- 2.22 (m, 1H), 2.67-2.72 (m, 2H), 3.34-3.41 (m, 2H), 3.60-3.66 (m, 1H), 3.75 (t, J = 6 Hz, 1H), 3.85 (t, J = 7.6 Hz, 1H), 3.94 (t, J = 8.4 Hz, 1H), 4.03-4.04 (m, 1H), 4.42-4.49 (m, 1H), 4.65 (s, 1H), 4.99 (d, J = 5.2 Hz, 1H), 5.15 (s, 2H), 6.43 (d, J = 6.8 Hz, 1H), 6.52 (d, J = 8 Hz, 1H), 6.75 (m, 2H), 6.82-6.89 (m, 1H), 6.99-7.03 (m, 1H), 7.10 (bs, 1H), 7.24-7.34 (m, 5H), 9.88 (s, 1H). LCMS (Method A): 2.04 min, MS: ES+ 495.2 (M + 1). HPLC (Method N): 7.68 min. |
| | (S)-(2-(Benzyloxy)-4-(difluoromethyl)-6- | |
| | hydroxyphenyl)(8-((tetrahydrofuran-3- | |
| | yl)amino)-3,4-dihydroisoquinolin-2(1H)- | |
| | yl)methanone | |
| | Prepared from 2-(benzyloxy)-4- | |
| | (difluoromethyl)-6-hydroxybenzoic acid | |
| | (Intermediate D) and (S)-N-(tetrahydrofuran- | |
| | 3-yl)-1,2,3,4-tetrahydroisoquinolin-8-amine | |
| | hydrochloride (Intermediate AA) | |
| 4.7 | 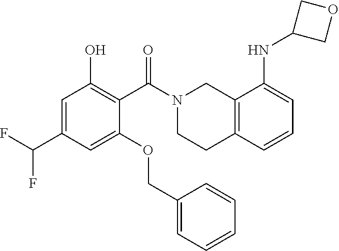 [SMILES: O=C(c1c(O)cc(C(F)F)cc1OCc1ccccc1)N1CCc2cccc(NC3COC3)c2C1] | HT ¹H NMR (DMSO-d6, 400 MHz, 349.6K): δ 2.67-2.73 (m, 2H), 3.38- 3.42 (m, 2H), 4.52-4.56 (m, 4H), 4.70 (s, 1H), 4.87 (s, 2H), 5.15 (s, 2H), 5.51 (bs, 1H), 6.17 (d, J = 8 Hz, 1H), 6.46 (d, J = 7.2 Hz, 1H), 6.74 (d, J = 4.4 Hz, 2H), 6.83-6.90 (m, 1H), 6.98 (t, J = 7.6 Hz, 1H), 7.24-7.34 (m, 5H), 9.87 (bs, 1H). LCMS (Method A): 1.983 min, MS: ES+ 481.08 (M + 1). HPLC (Method N): 7.35 min. |
| | (2-(Benzyloxy)-4-(difluoromethyl)-6- | |
| | hydroxyphenyl)(8-(oxetan-3-ylamino)-3,4- | |
| | dihydroisoquinolin-2(1H)-yl) methanone | |
| | Prepared from 2-(benzyloxy)-4- | |
| | (difluoromethyl)-6-hydroxybenzoic acid | |
| | (Intermediate D) and N-(oxetan-3-yl)-1,2,3,4- | |
| | tetrahydroisoquinolin-8-amine TFA salt | |
| | (Intermediate NC) | |
| 4.8 | 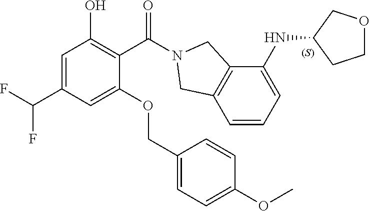 | HT ¹H NMR (DMSO-d6, 400 MHz, 348K): δ 1.70-1.95 (m, 2H), 2.10- 2.30 (m, 2H), 3.50-3.57 (m, 1H), 3.65-3.7 (m, 1H), 3.74-3.8 (m, 1H), 3.85-3.90 (m, 1H), 3.92-4.00 (m, 1H), 4.00-4.15 (m, 1H), 4.38 (s, 2H), 4.64 (s, 1H), 4.72 (s, 1H), 5.08 (s, 2H), 5.09-5.14 (m, 1H, D₂O exchangeable), 6.49-6.21 (m, 2H), 6.75-6.91 (m, 4H), 7.05-7.17 (m, 2H), 7.24-7.27 (m, 2H), 9.91 (m, 1H) LCMS (Method X): 6.413 min, MS: ES+ 511.3 (M + 1). |
| | (S)-(4-(Difluoromethyl)-2-hydroxy-6-((4- | HPLC (Method N): 7.254 min. |
| | methoxybenzyl)oxy)phenyl)(4- | |
| | ((tetrahydrofuran-3-yl)amino)isoindolin-2- | |
| | yl)methanone | |
| | Prepared from (S)-N-(tetrahydrofuran-3- | |
| | yl)isoindolin-4-amine hydrochloride | |
| | (Intermediate A) and 4-(difluoromethyl)-2- | |
| | hydroxy-6-((4-methoxybenzyl)oxy)benzoic | |
| | acid (Intermediate DC) | |
| 4.9 | 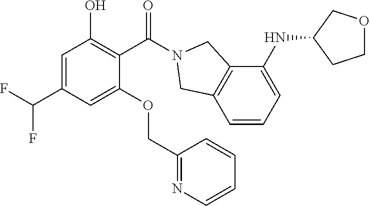 [SMILES: O=C(c1c(O)cc(C(F)F)cc1OCc1ccccn1)N1Cc2cccc(N[C@H]3CCOC3)c2C1] | ¹H NMR (DMSO-d6, 400 MHz): δ 1.75-2.19 (m, 4H), 3.48-3.98 (m, 4H), 4.39 (s, 1H), 4.46 (s, 1H), 4.64 (s, 1H), 4.47 (s, 1H), 5.24 (d, J = 3.2 Hz, 2H), 5.36-5.38 (m, 1H), 6.43- 6.64 (m, 2H), 6.76-6.82 (m, 2H), 6.94-7.14 (m, 2H), 7.27 (dd, J = 2.4 Hz, J = 7.6 Hz, 1H), 7.37 (d, J = 8.0 Hz, 1H), 7.70-7.74 (m, 1H), 8.50- 8.52 (m, 1H), 10.30 (bs, 1H). LCMS (Method X): 4.727 min, MS: ES+ 482.3 (M + 1). HPLC (Method N): 4.913 min. |
| | (S)-(4-(Difluoro methyl)-2-hydroxy-6- | |
| | (pyridin-2-ylmethoxy) phenyl) (4- | |
| | ((tetrahydrofuran-3-yl) amino) isoindolin-2- | |
| | yl) methanone | |
| | 4-(difluoromethyl)-2-hydroxy-6-(pyridin-2- | |
| | ylmethoxy)benzoic acid (Intermediate DB) | |
| | and (S)-N-(tetrahydrofuran-3-yl)isoindolin-4- | |
| | amine hydrochloride (Intermediate A) | |
| 4.10 | 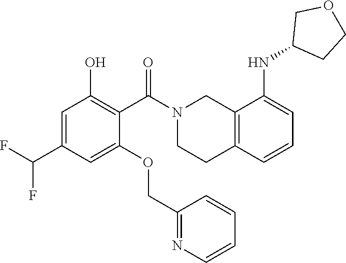 [SMILES: O=C(c1c(O)cc(C(F)F)cc1OCc1ccccn1)N1CCc2cccc(N[C@H]3CCOC3)c2C1] | HT ¹H NMR (DMSO-d6, 400 MHz, 348.9K): δ 1.92-2.23 (m, 2H), 2.76 (s, 2H), 3.31-4.04 (m, 7H), 4.50- 4.67 (m, 2H), 4.67-5.03 (m, 1H), 5.21 (s, 2H), 6.40-7.66 (m, 9H), 8.47-8.52 (m, 1H), 9.90 (bs, 1H). LCMS (Method A): 1.632 min, MS: ES+ 496.03 (M + 1). HPLC (Method N): 4.38 min. |
| | (S)-(4-(Difluoromethyl)-2-hydroxy-6-(pyridin- | |
| | 2-ylmethoxy)phenyl)(8-((tetrahydrofuran-3- | |
| | yl)amino)-3,4-dihydroisoquinolin-2(1H)- | |
| | yl)methanone | |
| | Prepared from 4-(Difluoromethyl)-2-hydroxy- | |
| | 6-(pyridin-2-ylmethoxy)benzoic acid | |
| | (Intermediate DB) and (S)-N- | |
| | (tetrahydrofuran-3-yl)-1,2,3,4- | |
| | tetrahydroisoquinolin-8-amine hydrochloride | |
| | (Intermediate AA) | |
| 4.11 | 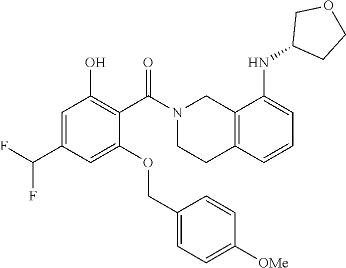 [SMILES: COc1ccc(COc2cc(C(F)F)cc(O)c2C(=O)N2CCc3cccc(N[C@H]4CCOC4)c3C2)cc1] | ¹H NMR (DMSO-d6, 400 MHz): δ 2.17-2.22 (m, 1H), 2.51-2.71 (m, 2H), 3.36-3.40 (m, 2H), 3.60-3.66 (m, 1H), 3.71 (s, 3H), 3.73-3.76 (m, 1H), 3.83-3.88 (m, 1H), 3.95 (t, J = 6.0 Hz, 14.0 Hz, 1H), 4.03-4.04 (m, 1H), 4.40-4.47 (m, 1H), 4.59-4.67 (m, 1H), 4.98 (d, J = 5.2 Hz, 1H), 5.05 (s, 2H), 6.41-6.55 (m, 2H), 6.69-6.75 (m, 2H), 6.81-6.91 (m, 3H), 6.95-7.09 (m, 2H), 7.26 (d, J = 8.4 Hz, 2H), 10.10 (s, 1H). LCMS (Method A): 2.037 min, MS: ES+ 525.0 (M + 1). HPLC (Method N): 8.35 min. |
| | (S)-(4-(Difluoromethyl)-2-hydroxy-6-((4- | |
| | methoxybenzyl)oxy)phenyl)(8- | |
| | ((tetrahydrofuran-3-yl)amino)-3,4- | |
| | dihydroisoquinolin-2(1H)-yl)methanone | |
| | Prepared from 4-(difluoromethyl)-2-hydroxy- | |
| | 6-((4-methoxybenzyl)oxy)benzoic acid | |
| | (Intermediate DC) and (S)-N- | |
| | (tetrahydrofuran-3-yl)-1,2,3,4- | |
| | tetrahydroisoquinolin-8-amine hydrochloride | |
| | (Intermediate AA) | |
| 4.12 | 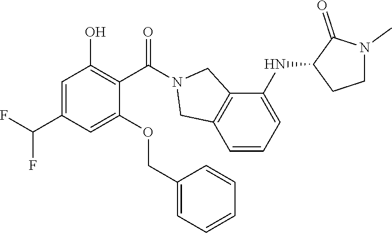 [SMILES: CN1CC[C@H](Nc2cccc3c2CN(C(=O)c2c(O)cc(C(F)F)cc2OCc2ccccc2)C3)C1=O] | ¹H NMR (DMSO-d6, 400 MHz): δ 1.66-1.89 (m, 1H), 2.36-2.51 (m, 1H), 2.79 (s, 3H), 3.26 (t, J = 8.4, 2H), 4.12-4.2 (m, 1H), 4.28-4.41 (m, 2H), 4.59-4.72 (m, 2H), 5.17 (bs, 2H), 5.42-5.54 (dd, J = 7.6 Hz, J = 8 Hz, 1H), 6.47-6.62 (m, 2H), 6.75-6.77 (m, 1H), 6.82-6.95 (m, 2H), 7.03-7.11 (m, 1H), 7.27-7.33 (m, 5H), 10.28 (bs, 1H). LCMS (Method F): 2.38 min, MS: ES+ 508.4 (M + 1). HPLC (Method N): 6.711 min. |
| | (S)-3-((2-(2-(Benzyloxy)-4-(difluoromethyl)- | |
| | 6-hydroxybenzoyl)isoindolin-4-yl)amino)-1- | |
| | methylpyrrolidin-2-one | |
| | Prepared from 2-(benzyloxy)-4- | |
| | (difluoromethyl)-6-hydroxybenzoic acid | |
| | (Intermediate D) and (S)-3-(isoindolin-4- | |
| | ylamino)-1-methylpyrrolidin-2-one | |
| | hydrochloride (Intermediate W) | |
| 4.13 | 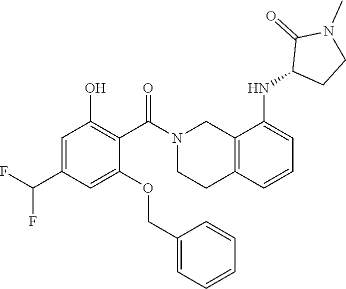 [SMILES: CN1CC[C@H](Nc2cccc3c2CN(C(=O)c2c(O)cc(C(F)F)cc2OCc2ccccc2)CC3)C1=O] | HT ¹H NMR (DMSO-d6, 400 MHz, 349K): δ 1.82-1.87 (m, 1H), 2.64- 2.75 (m, 3H), 2.81 (s, 3H), 3.99 (m, 1H), 4.12-4.20 (m, 1H), 4.61-4.65 (m, 1H), 4.97-5.08 (m, 1H), 5.16 (s, 2H), 6.40-6.61 (m, 2H), 6.70-6.90 (m, 3H), 6.94-7.01 (m, 1H), 7.21- 7.35 (m, 5H), 10.19 (bs, 1H). LCMS (Method A): 1.926 min, MS: ES+ 522 (M + 1). HPLC (Method N): 7.13 min. |
| | (S)-3-((2-(2-(benzyloxy)-4-(difluoromethyl)- | |
| | 6-hydroxybenzoyl)-1,2,3,4- | |
| | tetrahydroisoquinolin-8-yl)amino)-1- | |
| | methylpyrrolidin-2-one | |
| | Prepared from 2-(benzyloxy)-4- | |
| | (difluoromethyl)-6-hydroxybenzoic acid | |
| | (Intermediate D) and (S)-1-methyl-3- | |
| | ((1,2,3,4-tetrahydroisoquinolin-8- | |
| | yl)amino)pyrrolidin-2-one hydrochloride | |
| | (Intermediate ZC) | |
| 4.14 | 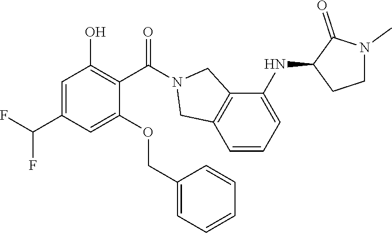 [SMILES: CN1CC[C@@H](Nc2cccc3c2CN(C(=O)c2c(O)cc(C(F)F)cc2OCc2ccccc2)C3)C1=O] | ¹H NMR (DMSO-d6, 400 MHz): δ 1.69-1.72 (m, 2H), 2.36-2.45 (m, 1H), 3.24-3.37 (m, 2H), 4.13-4.19 (m, 1H), 4.28-4.41 (m, 2H), 4.60- 4.72 (m, 2H), 5.18 (bs, 2H), 5.41- 5.54 (dd, J = 7.6 Hz, J = 8 Hz, 1H, D₂O exchangeable), 6.48-6.62 (m, 2H), 6.75-6.84 (m, 2H), 6.95-7.11 (m, 2H), 7.25-7.33 (m, 5H), 10.28 (bs, 1H). LCMS (Method X): 6.057 min, MS: ES+ 508 (M + 1). HPLC (Method N): 6.733 min. |
| | (R)-3-((2-(2-(Benzyloxy)-4-(difluoromethyl)- | |
| | 6-hydroxybenzoyl)isoindolin-4-yl)amino)-1- | |
| | methylpyrrolidin-2-one | |
| | Prepared from 2-(benzyloxy)-4- | |
| | (difluoromethyl)-6-hydroxybenzoic acid | |
| | (Intermediate D) and (R)-3-(isoindolin-4- | |
| | ylamino)-1-methylpyrrolidin-2-one | |
| | hydrochloride (Intermediate X) | |
| 4.15 | 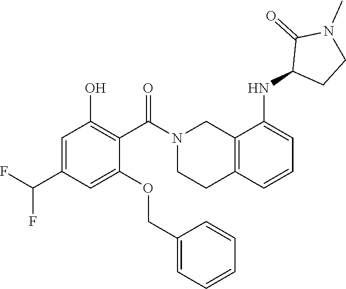 [SMILES: CN1CC[C@@H](Nc2cccc3c2CN(C(=O)c2c(O)cc(C(F)F)cc2OCc2ccccc2)CC3)C1=O] | ¹H NMR (DMSO-d6, 400 MHz): δ 1.82-1.87 (m, 1H), 2.64-2.75 (m, 3H), 2.81 (s, 3H), 3.24-3.40 (m, 1H), 3.99-4.01 (m, 1 H), 4.12-4.20 (m, 1H), 4.43-4.44 (d, J = 3.2 Hz, 1H), 4.55-4.65 (m, 1H), 5.02-5.09 (m, 1H), 5.16 (s, 2H), 6.42-6.60 (m, 2H), 6.70-6.83 (m, 2H), 6.93- 7.02 (m, 2H), 7.11-7.15 (m, 1H), 7.20-7.33 (m, 5H), 10.20 (bs, 1H). LCMS (Method A): 1.921 min, MS: ES+ 522 (M + 1). HPLC (Method N): 7.11 min. |
| | (R)-3-((2-(2-(Benzyloxy)-4-(difluoromethyl)- | |
| | 6-hydroxybenzoyl)-1,2,3,4- | |
| | tetrahydroisoquinolin-8-yl)amino)-1- | |
| | methylpyrrolidin-2-one | |
| | Prepared from 2-(benzyloxy)-4- | |
| | (difluoromethyl)-6-hydroxybenzoic acid | |
| | (Intermediate D) and (R)-1-methyl-3- | |
| | ((1,2,3,4-tetrahydroisoquinolin-8- | |
| | yl)amino)pyrrolidin-2-one hydrochloride | |
| | (Intermediate ZD) | |
| 4.16 | 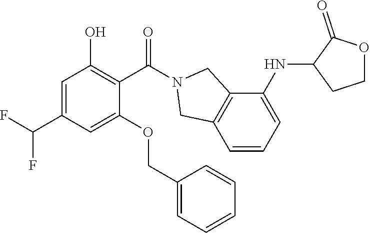 [SMILES: O=C1OCCC1Nc1cccc2c1CN(C(=O)c1c(O)cc(C(F)F)cc1OCc1ccccc1)C2] | ¹H NMR (DMSO-d6, 400 MHz): δ 1.98-2.08 (m, 1H), 2.22-2.33 (m, 1H), 4.18-4.72 (m, 7H), 5.18 (d, J = 4.8 Hz, 2H), 5.58-5.76 (m, 1H, D₂O exchangeable), 6.52-6.67 (m, 2H), 6.75 (d, J = 6 Hz, 1H), 6.83-6.95 (m, 2H), 7.06-7.14 (m, 1H), 7.23- 7.33 (m, 5H), 10.27 (s, 1H). LCMS (Method X): 6.106 min, MS: ES+ 495.3 (M + 1). HPLC (Method N): 6.925 min. |
| | 3-((2-(2-(Benzyloxy)-4-(difluoromethyl)-6- | |
| | hydroxybenzoyl)isoindolin-4- | |
| | yl)amino)dihydrofuran-2(3H)-one | |
| | Prepared from 2-(benzyloxy)-4- | |
| | (difluoromethyl)-6-hydroxybenzoic acid | |
| | (Intermediate D) and 3-(isoindolin-4- | |
| | ylamino)dihydrofuran-2(3H)-one | |
| | hydrochloride (Intermediate ZE) | |
| 4.17 | 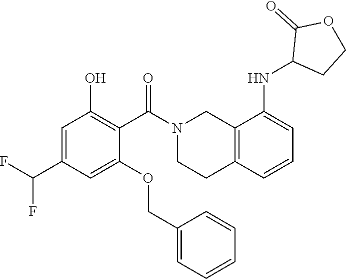 [SMILES: O=C1OCCC1Nc1cccc2c1CN(C(=O)c1c(O)cc(C(F)F)cc1OCc1ccccc1)CC2] | ¹H NMR (DMSO-d6, 400 MHz): δ 2.18-2.29 (m, 1H), 2.60-2.70 (m, 2H), 3.32-3.41 (m, 2H), 4.19-4.29 (m, 2H), 4.38-4.49 (m, 2H), 4.58- 4.69 (m, 2H), 5.15 (s, 2H), 5.42 (d, J = 8 Hz, 1H, D₂O exchangeable), 6.44 (d, J = 7.2 Hz, 1H), 6.53-6.64 (m, 1H), 6.70-6.83 (m, 2H), 6.95- 7.09 (m, 2H), 7.24-7.35 (m, 5H), 10.19 (s, 1H). LCMS (Method A): 1.988 min, MS: ES+ 509 (M + 1). HPLC (Method C): 5.32 min, |
| | 3-((2-(2-(Benzyloxy)-4-(difluoromethyl)-6- | |
| | hydroxybenzoyl)-1,2,3,4- | |
| | tetrahydroisoquinolin-8-yl) amino) | |
| | dihydrofuran-2(3H)-one | |
| | Prepared from of 2-(benzyloxy)-4- | |
| | (difluoromethyl)-6-hydroxybenzoic acid | |
| | (Intermediate D) and 3-((1,2,3,4- | |
| | tetrahydroisoquinolin-8-yl) amino) | |
| | dihydrofuran-2(3H)-one hydrochloride | |
| | (Intermediate ZF) | |
| 4.18 | 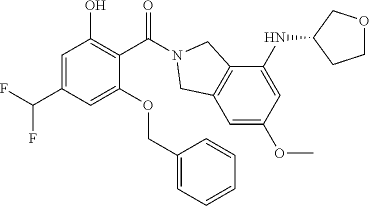 [SMILES: COc1cc2c(c(N[C@H]3CCOC3)c1)CN(C(=O)c1c(O)cc(C(F)F)cc1OCc1ccccc1)C2] | ¹H NMR (DMSO-d6, 400 MHz): δ 1.75-1.86 (m, 1H), 2.10-2.19 (m, 1H), 3.48-3.73 (m, 5H), 3.75-4.03 (m, 3H), 4.23-4.35 (m, 2H), 4.54- 4.70 (m, 2H), 5.17 (s, 2H), 5.53 (d, J = 5.6 Hz, 1H, D₂O exchangeable), 5.97 (d, J = 12.4 Hz, 1H), 6.09-6.22 (m, 1H), 6.74-7.09 (m, 3H), 7.27- 7.32 (m, 5H), 10.25 (d, J = 9.6 Hz, 1H). LCMS (Method A): 1.948 min, MS ES+ : 511 (M + 1). HPLC (Method N): 7.926 min |
| | (S)-(2-(Benzyloxy)-4-(difluoromethyl)-6- | |
| | hydroxyphenyl)(6-methoxy-4- | |
| | ((tetrahydrofuran-3-yl)amino)isoindolin-2- | |
| | yl)methanone | |
| | Prepared from 2-(benzyloxy)-4- | |
| | (difluoromethyl)-6-hydroxybenzoic acid | |
| | (Intermediate D) and (S)-6-methoxy-N- | |
| | (tetrahydrofuran-3-yl)isoindolin-4-amine | |
| | hydrochloride (Intermediate GA) | |
| 4.19 | 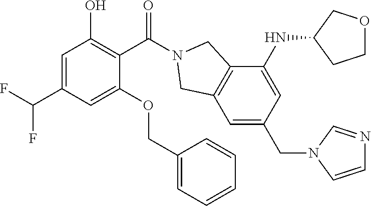 [SMILES: O=C(c1c(O)cc(C(F)F)cc1OCc1ccccc1)N1Cc2cc(Cn3ccnc3)cc(N[C@H]3CCOC3)c2C1] | HT ¹H NMR (DMSO-d6, 400 MHz, 349K): δ 1.74-1.86 (m, 2H), 2.09- 2.20 (m, 1H), 3.45-4.04 (m, 5H), 4.37 (s, 2H), 4.62-4.69 (m, 2H), 5.06-5.15 (m, 4H), 5.29-5.38 (m, 1H, D₂O exchangeable), 6.37-6.54 (m, 2H), 6.76 (d, J = 4 Hz, 1H), 6.84 -6.90 (m, 3H), 7.04-7.14 (m, 1H), 7.24-7.33 (m, 5H), 7.65-7.70 (m, 1H), 9.95 (s, 1H). LCMS (Method A): 1.350 min, MS: ES+ 561.1 (M + 1). HPLC (Method N): 5.400 min |
| | (S)-(6-((1H-Imidazol-1-yl) methyl)-4- | |
| | ((tetrahydrofuran-3-yl) amino) isoindolin-2- | |
| | yl) (2-(benzyloxy)-4-(difluoromethyl)-6- | |
| | hydroxyphenyl)methanone | |
| | Prepared from 2-(benzyloxy)-4- | |
| | (difluoromethyl)-6-hydroxybenzoic acid | |
| | (Intermediate D) and (S)-6-((1H-imidazol-1- | |
| | yl)methyl)-N-(tetrahydrofuran-3-yl)isoindolin- | |
| | 4-amine hydrochloride (Intermediate ZG) | |
| 4.20 | 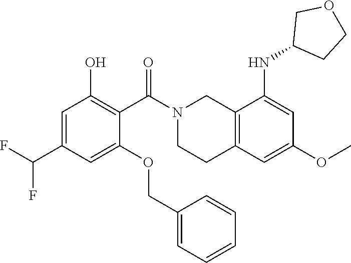 [SMILES: COc1cc2c(c(N[C@H]3CCOC3)c1)CN(C(=O)c1c(O)cc(C(F)F)cc1OCc1ccccc1)CC2] | ¹H NMR (DMSO-d6, 400 MHz): δ 1.27 (s, 1H), 1.94 (bs, 1H), 2.21- 2.25 (m, 1H), 2.68 (m, 2H), 3.39 (d, J = 18.8 Hz, 2H), 3.66-3.95 (m, 7H), 3.97-4.38 (m, 2H), 4.41-4.45 (m, 1H), 4.66-4.64 (m, 3H), 4.88 (bs, 1H), 5.15 (s, 2H), 6.03-6.07 (m, 2H), 6.70-6.82 (m, 2H), 6.95-7.09 (m, 1H), 7.25-7.36 (m, 5H), 9.97 (bs, 1H). LCMS (Method A): 2.008 min, MS: ES+ 525 (M + 1). HPLC (Method N): 8.047 min. |
| | (S)-(2-(Benzyloxy)-4-(difluoromethyl)-6- | |
| | hydroxyphenyl)(6-methoxy-8- | |
| | ((tetrahydrofuran-3-yl)amino)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and (S)-6-Methoxy-N- | |
| | (tetrahydrofuran-3-yl)-1,2,3,4- | |
| | tetrahydroisoquinolin-8-amine hydrochloride | |
| | (Intermediate ZH) | |
| 4.21 | 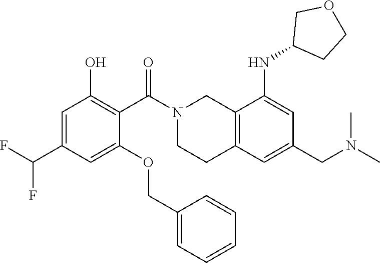 [SMILES: CN(C)Cc1cc2c(c(N[C@H]3CCOC3)c1)CN(C(=O)c1c(O)cc(C(F)F)cc1OCc1ccccc1)CC2] | ¹H NMR (DMSO-d6, 400 MHz): δ 1.90-1.94 (m, 1H), 2.33 (s, 6H), 2.50-2.69 (m, 1H), 3.33-4.04 (m, 8H), 4.41-4.49 (m, 1H), 4.59-4.67 (m, 1H), 4.99 (m, 1H, D₂O exchangeable), 5.14 (s, 2H), 6.35 (s, 1H), 6.44 (s, 1H), 6.73-6.82 (m, 2H), 6.95-7.24 (m, 1H), 7.28-7.34 (m, 5H), 10.25 (bs, 1H). LCMS (Method A): 1.401 min, MS ES+ : 552.0 (M + 1). HPLC (Method B): 5.463 min. |
| | (S)-(2-(Benzyloxy)-4-(difluoromethyl)-6- | |
| | hydroxyphenyl)(6-((dimethylamino)methyl)- | |
| | 8-((tetrahydrofuran-3-yl)amino)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and (S)-6- | |
| | ((dimethylamino)methyl)-N-(tetrahydrofuran- | |
| | 3-yl)-1,2,3,4-tetrahydroisoquinolin-8-amine | |
| | hydrochloride (Intermediate ZI) | |
| 4.22 | 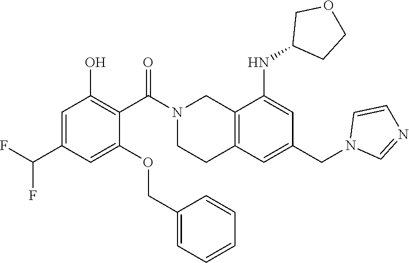 [SMILES: O=C(c1c(O)cc(C(F)F)cc1OCc1ccccc1)N1CCc2cc(Cn3ccnc3)cc(N[C@H]3CCOC3)c2C1] | ¹H NMR (DMSO-d6, 400 MHz): δ 1.88-1.91 (m, 1H), 2.14-2.21 (m, 1H), 2.62-2.67 (m, 2H), 3.55-3.62 (m, 1H), 3.73-3.77 (m, 1H), 3.82- 3.87 (m, 1H), 3.92-4.00 (m, 2H), 4.40-4.47 (m, 1H), 4.56-4.64 (m, 1H), 5.11 (s, 2H), 5.13 (s, 2H), 6.30 (s, 1H), 6.42 (s, 1H), 6.69-6.74 (m, 1H), 6.80 (d, J = 5.6 Hz, 1H), 6.88 (s, 1H), 6.94-7.022 (m, 1H), 7.17 (s, 1H), 7.21-7.26 (m, 3H), 7.30-7.33 (m, 2H), 7.72 (s, 1H), 10.19 (s, 1H). LCMS (Method A): 1.391 min, MS: ES+ 575.1 (M + 1). HPLC (Method N): 5.468 min. |
| | (S)-(6-((1H-Imidazol-1-yl)methyl)-8- | |
| | ((tetrahydrofuran-3-yl)amino)-3,4- | |
| | dihydroisoquinolin-2(1H)-yl)(2-(benzyloxy)- | |
| | 4-(difluoromethyl)-6- | |
| | hydroxyphenyl)methanone | |
| | Prepared from 2-(benzyloxy)-4- | |
| | (difluoromethyl)-6-hydroxybenzoic acid | |
| | (Intermediate D) and (S)-6-((1H-imidazol-1- | |
| | yl)methyl)-N-(tetrahydrofuran-3-yl)-1,2,3,4- | |
| | tetrahydroisoquinolin-8-amine hydrochloride | |
| | (Intermediate ZJ) | |
| 4.23 | 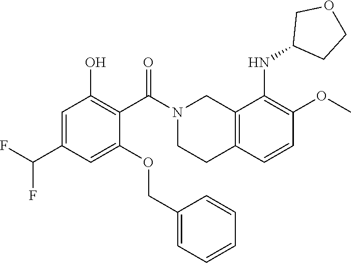 [SMILES: COc1ccc2c(c1N[C@H]1CCOC1)CN(C(=O)c1c(O)cc(C(F)F)cc1OCc1ccccc1)CC2] | ¹H NMR (DMSO-d6, 400 MHz): δ 1.44 (s, 1H), 1.65-1.79 (m, 1H), 2.74 (d, J = 18.8 Hz, 2H), 3.27-4.00 (m, 11H), 4.32 (s, 1H), 4.65-4.79 (m, 1H), 4.96-5.13 (m, 2H), 6.68- 6.88 (m, 4H), 6.95-7.125 (m, 2H), 7.17-7.28 (m, 4H), 9.83 (s, 1H). LCMS (Method A): 1.923 min, MS: ES+ 525.2 (M + 1). HPLC (Method N): 7.247 min. |
| | (S)-(2-(Benzyloxy)-4-(difluoromethyl)-6- | |
| | hydroxyphenyl)(7-methoxy-8- | |
| | ((tetrahydrofuran-3-yl)amino)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and (S)-7-methoxy-N- | |
| | (tetrahydrofuran-3-yl)-1,2,3,4- | |
| | tetrahydroisoquinolin-8-amine hydrochloride | |
| | (Intermediate ZK) | |
| 4.24 | 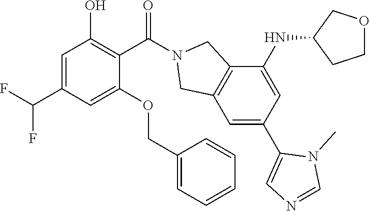 [SMILES: Cn1cncc1-c1cc2c(c(N[C@H]3CCOC3)c1)CN(C(=O)c1c(O)cc(C(F)F)cc1OCc1ccccc1)C2] | HT ¹H NMR (DMSO-d6, 400 MHz, 348K): δ 1.92-1.98 (m, 1H), 2.19- 2.22 (m, 1H), 3.54-3.57 (m, 1H), 3.64-3.70 (m, 4H), 3.75-3. 98 (m, 3H), 4.42-4.43 (m, 1H), 4.45-4.46 (m, 2H), 4.69-4.77 (m, 2H), 5.17 (d, J = 2.8 Hz, 2H), 5.35-5.45 (m, 1H, D₂O exchangeable), 6.49-6.73 (m, 2H), 6.78-7.05 (m, 4H), 7.23-7.37 (m, 5H), 7.64-7.68 (m, 1H), 9.98 (d, J = 4 Hz, 1H). ¹H NMR is complex in the aliphatic region due to rotamers. LCMS (Method A): 1.380 min, MS: ES+ 561.3 (M + 1). |
| | (S)-(2-(Benzyloxy)-4-(difluoromethyl)-6- | HPLC (Method N): 5.38 min. |
| | hydroxyphenyl)(6-(1-methyl-1H-imidazol-5- | |
| | yl)-4-((tetrahydrofuran-3-yl)amino)isoindolin- | |
| | 2-yl)methanone | |
| | Prepared from 2-(benzyloxy)-4- | |
| | (difluoromethyl)-6-hydroxybenzoic acid | |
| | (Intermediate D) and (S)-6-(1-methyl-1H- | |
| | imidazol-5-yl)-N-(tetrahydrofuran-3- | |
| | yl)isoindolin-4-amine hydrochloride | |
| | (Intermediate ZL) | |
| 4.25 | 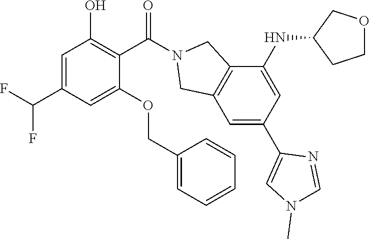 [SMILES: Cn1cnc(-c2cc3c(c(N[C@H]4CCOC4)c2)CN(C(=O)c2c(O)cc(C(F)F)cc2OCc2ccccc2)C3)c1] | HT ¹H NMR (DMSO-d6, 400 MHz, 349K): δ 1.82-1.99 (m, 1H), 2.17- 2.22 (m, 1H), 3.52-3.54 (m, 1H), 3.63-3.72 (m, 3H), 3.74-3.80 (m, 1H), 3.89-4.07 (m, 2H), 4.38-4.43 (m, 2H), 4.65-4.74 (m, 2H), 5.17 (s, 2H), 6.77 (s, 1H), 6.85-6.91 (m, 3H), 7.03-7.05 (m, 1H), 7.24-7.30 (m, 3H), 7.34-7.41 (m, 2H), 7.49- 7.57 (m, 2H), 9.97 (s, 1H). LCMS (Method A): 1.430 min, MS: ES+ 561.1 (M + 1). HPLC (Method N): 4.64 min. |
| | (S)-(2-(Benzyloxy)-4-(difluoromethyl)-6- | |
| | hydroxyphenyl)(6-(1-methyl-1H-imidazol-4- | |
| | yl)-4-((tetrahydrofuran-3-yl)amino)isoindolin- | |
| | 2-yl)methanone | |
| | Prepared from 2-(benzyloxy)-4- | |
| | (difluoromethyl)-6-hydroxybenzoic acid | |
| | (Intermediate D) and (S)-6-(1-methyl-1H- | |
| | imidazol-4-yl)-N-(tetrahydrofuran-3- | |
| | yl)isoindolin-4-amine hydrochloride | |
| | (Intermediate ZM) | |
| 4.26 | 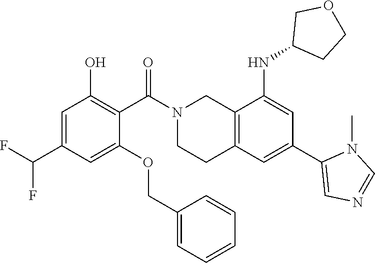 [SMILES: Cn1cncc1-c1cc2c(c(N[C@H]3CCOC3)c1)CN(C(=O)c1c(O)cc(C(F)F)cc1OCc1ccccc1)CC2] | ¹H NMR (DMSO-d6, 400 MHz): δ 1.96 (bs, 1H), 2.21-2.28 (m, 1H), 2.67-2.77 (m, 2H), 3.41-3.48 (m, 2H), 3.66 (s, 3H), 3.74-3.83 (m, 1H), 3.88-3.93 (m, 1H), 3.98 (t, J = 7.6 Hz, J = 6.8 Hz, 1H), 4.15 (bs, 1H), 4.30 (s, 1H), 4.51-4.58 (m, 1H), 4.67-4.74 (m, 1H), 4.94-5.03 (m, 1H), 5.15 (s, 1H), 6.53 (s, br, 2H), 6.75 (s, 1H), 6.83 (s, br, 1H), 6.89-7.03 (s, 2H), 7.13-7.36 (m, 5H), 7.63 (s, 1H), 9.89 (bs, 1H). LCMS (Method A): 1.360 min, MS: ES+ 575.3 (M + 1). HPLC (Method N): 5.50 min. |
| | (S)-(2-(Benzyloxy)-4-(difluoromethyl)-6- | |
| | hydroxyphenyl) (6-(1-methyl-1H-imidazol-5- | |
| | yl)-8-((tetrahydrofuran-3-yl) amino)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and S)-6-(1-methyl-1H- | |
| | imidazol-5-yl)-N-(tetrahydrofuran-3-yl)- | |
| | 1,2,3,4-tetrahydroisoquinolin-8-amine | |
| | hydrochloride (Intermediate ZN) | |
| 4.27 | 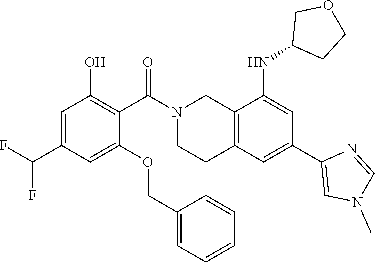 [SMILES: Cn1cnc(-c2cc3c(c(N[C@H]4CCOC4)c2)CN(C(=O)c2c(O)cc(C(F)F)cc2OCc2ccccc2)CC3)c1] | ¹H NMR (DMSO-d6, 400 MHz): δ 1.93-1.95 (m, 2H), 2.21-2.25 (m, 1H), 2.68-2.73 (m, 2H), 3.62-3.67 (m, 5H), 3.76-3.80 (m, 1H), 3.85- 3.89 (m, 1H), 3.97-4.02 (m, 1H), 4.11-4.12 (m, 1H), 4.45-4.49 (m, 1H), 4.63-4.66 (m, 1H), 4.99-5.01 (m, 1H, D₂O exchangeable), 5.15 (s, 2H), 6.84-6.91 (m, 4H), 6.95 (s, 1H), 7.01-7.18 (m, 1H), 7.23-7.35 (m, 5H), 7.56 (s, 1H), 7.63 (s, 1H), 10.21 (s, 1H). LCMS (Method A): 1.372 min, MS: ES+ 575.1 (M + 1). HPLC (Method N): 5.456 min. |
| | (S)-(2-(Benzyloxy)-4-(difluoromethyl)-6- | |
| | hydroxyphenyl) (6-(1-methyl-1H-imidazol-4- | |
| | yl)-8-((tetrahydrofuran-3-yl) amino)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and (S)-6-(1-methyl-1H- | |
| | imidazol-4-yl)-N-(tetrahydrofuran-3-yl)- | |
| | 1,2,3,4-tetrahydroisoquinolin-8-amine | |
| | hydrochloride (Intermediate ZO) | |
| 4.28 | 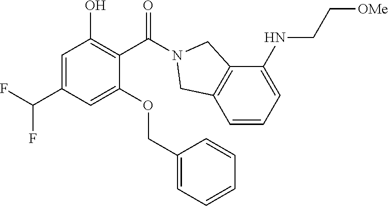 [SMILES: COCCNc1cccc2c1CN(C(=O)c1c(O)cc(C(F)F)cc1OCc1ccccc1)C2] | HT ¹H NMR (DMSO-d6, 400 MHz, 349K): δ 3.09-3.12 (m, 1H), 3.20- 3.24 (m, 1H), 3.31 (t, J = 6 Hz, 2H), 3.44 (t, J = 6.4 Hz, 1H), 3.5 (t, J = 6 Hz, 1H), 4.35-4.42 (m, 2H), 4.63- 4.72 (m, 3H), 5.05 (m, 1H, D₂O exchangeable), 5.17 (s, 2H), 6.45- 6.61 (m, 2H), 6.77 (d, J = 5.2 Hz, 1H), 6.84 (s, 1H), 6.90-7.13 (m, 2H), 7.24-7.35 (m, 5H), 9.95 (s, 1H). LCMS (Method A): 1.990 min, MS: ES+ 469.2 (M + 1). HPLC (Method N): 7.401 min |
| | (2-(Benzyloxy)-4-(difluoromethyl)-6- | |
| | hydroxyphenyl) (4-((2- | |
| | methoxyethyl)amino)isoindolin-2- | |
| | yl)methanone | |
| | Prepared from 2-(benzyloxy)-4- | |
| | (difluoromethyl)-6-hydroxybenzoic acid | |
| | (Intermediate D) and N-(2- | |
| | methoxyethyl)isoindolin-4-amine | |
| | hydrochloride (Intermediate ZP) | |
| 4.29 | 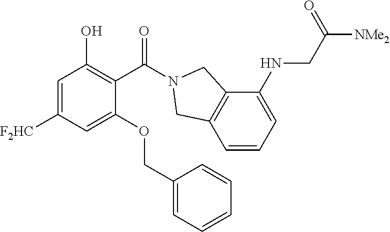 [SMILES: CN(C)C(=O)CNc1cccc2c1CN(C(=O)c1c(O)cc(C(F)F)cc1OCc1ccccc1)C2] | ¹H NMR (DMSO-d6, 400 MHz): δ 2.81-3.04 (m, 6H), 3.81-3.86 (m, 1H), 3.95 (d, J = 5.6 Hz, 1H), 4.31- 4.43 (m, 2H), 4.63 (s, 1H), 4.75 (d, J = 15.6 Hz, 1H), 5.17 (s, br, 2H), 5.16-5.36 (m, 1H, D₂O exchangeable), 6.40-6.76 (m, 3H), 6.81-6.96 (m, 2H), 7.04-7.12 (m, 1H), 7.23-7.35 (m, 5H), 10.30 (s, 1H). LCMS (Method A): 1.866 min, MS: ES+ 495.98 (M + 1). HPLC (Method N): 6.86 min. |
| | 2-((2-(2-(Benzyloxy)-4-(difluoro methyl)-6- | |
| | hydroxybenzoyl) isoindolin-4-yl) amino)-N, | |
| | N-dimethylacetamide | |
| | Prepared from 2-(benzyloxy)-4- | |
| | (difluoromethyl)-6-hydroxybenzoic acid | |
| | (Intermediate D) and 2-(isoindolin-4- | |
| | ylamino)-N,N-dimethylacetamide | |
| | hydrochloride (Intermediate ZQ) | |
| 4.30 | 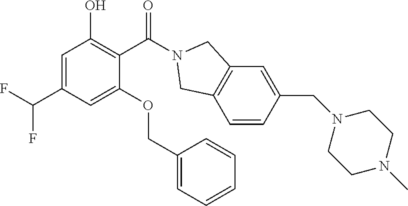 [SMILES: CN1CCN(Cc2ccc3c(c2)CN(C(=O)c2c(O)cc(C(F)F)cc2OCc2ccccc2)C3)CC1] | ¹H NMR (DMSO-d6, 400 MHz): δ 2.60-2.76 (m, 4H), 2.90-3.24 (m, 4H), 3.52 (d, J = 15.2 Hz, 2H), 4.46 (d, J = 12 Hz, 2H), 4.77 (s, 2H), 5.17 (s, 2H), 6.77 (s, 1H), 6.82-7.10 (m, 2H) 7.18-7.38 (m, 7H), 10.33 (bs, 1H), 10.34 (s, 1H). ¹H NMR (CD₃CN-d6, 400 MHz): δ 1.29 (s, 2H), 2.63 (d, J = 7.6 Hz, 4H), 2.88 (bs, 4H), 3.20 (bs, 4H), 3.60 (d, J = 13.2 Hz, 2H), 4.56 (s, 2H), 4.85 (s, 2H), 5.18 (d, J = 1.6 Hz, 2H), 6.60- 6.88 (m, 4H), 7.17 (d, J = 7.2 Hz, 1H), 7.25-7.29 (m, 4H), 7.34-7.35 |
| | (2-(Benzyloxy)-4-(difluoromethyl)-6- | (m, 3H). |
| | hydroxyphenyl) (5-((4-methylpiperazin-1-yl) | LCMS (Method A): 1.313 min, MS: |
| | methyl) isoindolin-2-yl) methanone | ES+ 507.9 (M + 1). |
| | Prepared from 2-(benzyloxy)-4- | HPLC (Method N): 4.121 min |
| | (difluoromethyl)-6-hydroxybenzoic acid | |
| | (Intermediate D) and 5-((4-methylpiperazin- | |
| | 1-yl)methyl)isoindoline hydrochloride | |
| | (Intermediate RA) | |
| 4.31 | 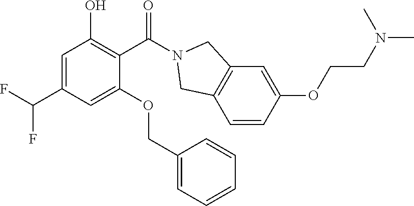 [SMILES: CN(C)CCOc1ccc2c(c1)CN(C(=O)c1c(O)cc(C(F)F)cc1OCc1ccccc1)C2] | HT ¹H NMR (DMSO-d6, 400 MHz, 349.4K): δ 2.30-2.34 (m, 6H), 2.73- 2.77 (m, 2H), 4.04-4.13 (m, 2H), 4.41-4.45 (m, 2H), 4.72-4.76 (m, 2H), 5.17 (s, 2H), 6.77-6.91 (m, 4H), 6.99-7.15 (m, 1H), 7.26-7.34 (m, 6H), 10.01 (bs, 1H). LCMS (Method A): 1.390 min, MS: ES+ 483.1 (M + 1). HPLC (Method N): 4.644 min. |
| | (2-(Benzyloxy)-4-(difluoromethyl)-6- | |
| | hydroxyphenyl)(5-(2- | |
| | (dimethylamino)ethoxy)isoindolin-2- | |
| | yl)methanone | |
| | Prepared from 2-(benzyloxy)-4- | |
| | (difluoromethyl)-6-hydroxybenzoic acid | |
| | (Intermediate D) and 2-(isoindolin-5-yloxy)- | |
| | N,N-dimethylethan-1-amine hydrochloride | |
| | (Intermediate ZR) | |
| 4.32 | 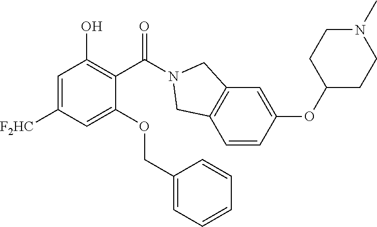 [SMILES: CN1CCC(Oc2ccc3c(c2)CN(C(=O)c2c(O)cc(C(F)F)cc2OCc2ccccc2)C3)CC1] | ¹H NMR (DMSO-d6, 400 MHz): δ 1.61-1.65 (m, 2H), 2.91-2.91 (m, 2H), 2.14-2.25 (m, 5H), 2.63-2.68 (m, 2H), 4.27-4.43 (m, 3H), 4.71 (d, J = 14.8 Hz, 2H), 5.17 (s, 2H), 6.74- 6.75 (m, 1H), 6.81-6.95 (m, 3H), 6.99-7.15 (m, 2H), 7.24-7.33 (m, 5H), 10.10-10.5 (m, 1H). LCMS (Method A): 1.445 min, MS ES+ : 509.1 (M + 1). HPLC (Method B): 5.54 min. |
| | (2-(Benzyloxy)-4-(difluoromethyl)-6- | |
| | hydroxyphenyl)(5-((1-methylpiperidin-4- | |
| | yl)oxy)isoindolin-2-yl)methanone | |
| | Prepared from 2-(benzyloxy)-4- | |
| | (difluoromethyl)-6-hydroxybenzoic acid | |
| | (Intermediate D) and 5-((1-methylpiperidin-4- | |
| | yl)oxy)isoindoline hydrochloride | |
| | (Intermediate V) | |
| 4.33 | 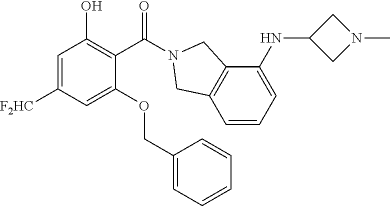 [SMILES: CN1CC(Nc2cccc3c2CN(C(=O)c2c(O)cc(C(F)F)cc2OCc2ccccc2)C3)C1] | ¹H NMR (DMSO-d6, 400 MHz): δ 2.26 (s, 1H), 2.33 (s, 1H), 2.87 (t, J = 5.6 Hz, 1H), 2.99 (t, J = 6.8 Hz, 1H), 3.89-3.94 (m, 1H), 3.99-4.03 (m, 1H), 4.31-4.42 (m, 2H), 4.62 (m, 1H), 4.70-4.71 (m, 1H), 5.16-5.17 (m, 2H), 5.68-5.83 (m, 1H, D₂O exchangeable), 6.27-6.33 (m, 1H), 6.48-6.63 (m, 1H), 6.76-6.94 (m, 2H), 6.94-7.11 (m, 2H), 7.24-7.38 (m, 5H) LCMS (Method A): 6.813 min, MS: ES+ 480.3 (M + 1). HPLC (Method P): 5.683 min. |
| | ((2-(benzyloxy)-4-(difluoromethyl)-6- | |
| | hydroxyphenyl)(4-((1-methylazetidin-3- | |
| | yl)amino)isoindolin-2-yl)methanone | |
| | Prepared from 2-(benzyloxy)-4- | |
| | (difluoromethyl)-6-hydroxybenzoic acid | |
| | (Intermediate D) and N-(1-methylazetidin-3- | |
| | yl) isoindolin-4-amine TFA salt (Intermediate | |
| | NA) | |
| 4.34 | 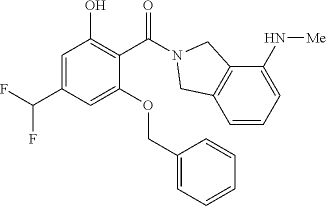 [SMILES: CNc1cccc2c1CN(C(=O)c1c(O)cc(C(F)F)cc1OCc1ccccc1)C2] | HT ¹H NMR (DMSO-d6, 400 MHz, 349K): δ 2.65-2.77 (m, 3H), 4.34- 4.73 (m, 4H), 5.17 (s, 2H), 6.38- 6.61 (m, 2H), 6.77-6.91 (m, 3H), 7.05-7.15 (m, 1H), 7.23-7.35 (m, 5H), 9.94 (s, 1H). LCMS (Method A): 2.003 min, MS: ES+ 425.06 (M + 1). HPLC (Method N): 7.141 min. |
| | (2-(Benzyloxy)-4-(difluoro methyl)-6- | |
| | hydroxyphenyl) (4-(methylamino) isoindolin- | |
| | 2-yl) methanone | |
| | Prepared from 2-(benzyloxy)-4- | |
| | (difluoromethyl)-6-hydroxybenzoic acid | |
| | (Intermediate D) and N-methylisoindolin-4- | |
| | amine hydrochloride (Intermediate ZS) | |
| 4.35 | 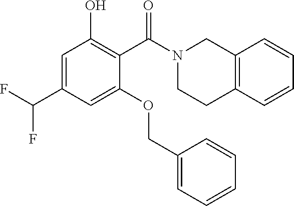 [SMILES: O=C(c1c(O)cc(C(F)F)cc1OCc1ccccc1)N1CCc2ccccc2C1] | ¹H NMR (DMSO-d6, 400 MHz): δ 2.73-2.81 (m, 2H), 3.40-3.59 (m, 2H), 4.37 (d, J = 8 Hz, 1H), 4.78 (s, 1H), 4.96-5.05 (m, 1H), 5.14 (s, 1H), 6.72 (d, J = 5.6 Hz, 1H), 6.78- 7.18 (m, 5H), 7.23 (m, 6H), 10.20 (s, 1H). LCMS (Method A): 2.024 min, MS: ES+ 410.0 (M + 1). HPLC (Method H): 8.610 min. |
| | (2-(Benzyloxy)-4-(difluoromethyl)-6- | |
| | hydroxyphenyl)(3,4-dihydroisoquinolin- | |
| | 2(1H)-yl)methanone | |
| | Prepared from 2-(benzyloxy)-4- | |
| | (difluoromethyl)-6-hydroxybenzoic acid | |
| | (Intermediate D) and 1,2,3,4- | |
| | tetrahydroisoquinoline (CAS: 91-21-4) | |
| 4.36 | 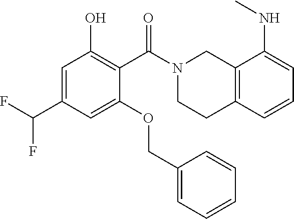 [SMILES: CNc1cccc2c1CN(C(=O)c1c(O)cc(C(F)F)cc1OCc1ccccc1)CC2] | HT ¹H NMR (DMSO-d6, 400 MHz, 348K): δ 2.61 (d, J = 4.8 Hz, 1H), 2.68-2.74 (m, 4H), 3.40-3.44 (m, 1H), 4.15 (s, 1H), 4.43 (d, J = 17.6 Hz, 1H), 4.58 (d, J = 17.2 Hz, 1H), 4.97-5.05 (m, 1H), 5.15 (s, 1H), 6.36-6.48 (m, 2H), 6.69-6.83 (m, 3H), 6.94-7.12 (m, 1H), 7.23-7.34 (m, 5H), 10.20 (s, 1H). LCMS (Method A): 2.089 min, MS: ES+ 439.1 (M + 1). HPLC (Method N): 8.108 min, |
| | (2-(Benzyloxy)-4-(difluoromethyl)-6- | |
| | hydroxyphenyl)(8-(methylamino)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and N-methyl-1,2,3,4- | |
| | tetrahydroisoquinolin-8-amine hydrochloride | |
| | (Intermediate ZT) | |
| 4.37 | 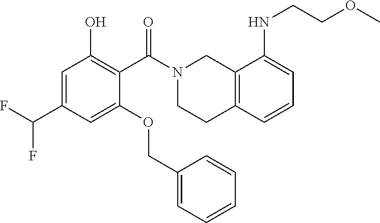 [SMILES: COCCNc1cccc2c1CN(C(=O)c1c(O)cc(C(F)F)cc1OCc1ccccc1)CC2] | ¹H NMR (DMSO-d6, 400 MHz): δ 2.50-2.51 (m, 2H), 2.67-2.73 (m, 2H), 3.21 (s, 1H), 3.30-3.41 (m, 5H), 3.59 (t, J = 5.6 Hz, 1H), 4.15 (s, 1H), 4.43-4.70 (m, 2H), 4.97-5.15 (m, 2H), 6.39-6.54 (m, 2H), 6.74- 6.89 (m, 3H), 7.00 (t, J = 12 Hz, 1H), 7.16-7.35 (m, 5H), 9.86 (s, 1H). LCMS (Method A): 2.101 min, MS: ES+ 483.1 (M + 1). HPLC (Method N): 8.437 min. |
| | (2-(Benzyloxy)-4-(difluoromethyl)-6- | |
| | hydroxyphenyl)(8-((2-methoxyethyl)amino)- | |
| | 3,4-dihydroisoquinolin-2(1H)-yl)methanone | |
| | Prepared from 2-(benzyloxy)-4- | |
| | (difluoromethyl)-6-hydroxybenzoic acid | |
| | (Intermediate D) and N-(2-methoxyethyl)- | |
| | 1,2,3,4-tetrahydroisoquinolin-8-amine | |
| | hydrochloride (Intermediate ZU) | |
| 4.38 | 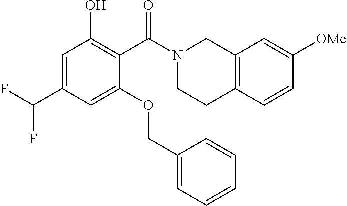 [SMILES: COc1ccc2c(c1)CN(C(=O)c1c(O)cc(C(F)F)cc1OCc1ccccc1)CC2] | ¹H NMR (DMSO-d6, 400 MHz): δ 2.68-2.73 (m, 2H), 3.35-3.40 (m, 1H), 3.66 (s, 1H), 3.75 (s, 2H), 4.36 (d, J = 4.8 Hz, 1H), 4.75 (d, J = 2.4 Hz, 1H), 5.03 (d, J = 9.2 Hz, 1H), 5.14 (bs, 1H), 6.79-6.86 (m, 4H), 6.95-7.03 (m, 1H), 7.08-7.12 (m, 2H), 7.22-7.28 (m, 4H), 10.18 (d, J = 13.2 Hz, 1H). LCMS (Method A): 2.058 min, MS: ES+ 440.1 (M + 1). HPLC (Method N): 8.50 min. |
| | (2-(Benzyloxy)-4-(difluoromethyl)-6- | |
| | hydroxyphenyl)(7-methoxy-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and 7-methoxy-1,2,3,4- | |
| | tetrahydroisoquinoline hydrochloride (CAS | |
| | 1745-05-7) | |
| 4.39 | 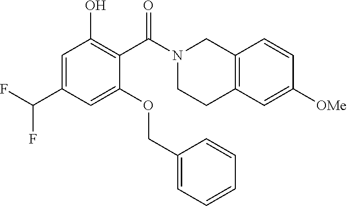 [SMILES: COc1ccc2c(c1)CCN(C(=O)c1c(O)cc(C(F)F)cc1OCc1ccccc1)C2] | ¹H NMR (DMSO-d6, 400 MHz): δ 2.68-2.89 (m, 2H), 3.37-3.43 (m, 1H), 3.53-3.72 (m, 1H), 3.72 (s, 3H), 4.69-4.74 (m, 1H), 5.96-5.14 (m, 1H), 5.36 (s, 1H), 6.68-6.71 (m, 2H), 6.72-6.82 (m, 2H), 6.95-6.96 (m, 1H), 7.09 (d, J = 6 Hz, 1H), 7.16-7.29 (m, 5H), 10.16-10.20 (m,1H). LCMS (Method A): 2.037 min, MS: ES+ 440.1 (M + 1). HPLC (Method N): 8.44 min. |
| | (2-(Benzyloxy)-4-(difluoromethyl)-6- | |
| | hydroxyphenyl)(6-methoxy-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and 6-methoxy-1,2,3,4- | |
| | tetrahydroisoquinoline (CAS: 42923-77-3) | |
| 4.40 | 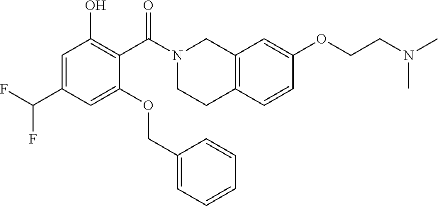 [SMILES: CN(C)CCOc1ccc2c(c1)CN(C(=O)c1c(O)cc(C(F)F)cc1OCc1ccccc1)CC2] | ¹H NMR (DMSO-d6, 400 MHz): δ 2.22-2.28 (m, 6H), 2.61-2.70 (m, 4H), 3.96 (t, J = 6 Hz, 1H), 4.07 (t, J = 5.6 Hz, 2H), 4.35-4.36 (m, 1H), 4.74 (bs, 1H), 4.98-5.07 (m, 1H), 5.14 (s, 1H), 6.74-6.87 (m, 4H), 6.95 (s, 1H), 7.11-7.27 (m, 5H), 10.20 (d, J = 13.6 Hz, 1H). LCMS (Method A): 1.456 min, MS ES+ : 497.3 (M + 1). HPLC (Method N): 5.615 min. |
| | (2-(Benzyloxy)-4-(difluoromethyl)-6- | |
| | hydroxyphenyl)(7-(2- | |
| | (dimethylamino)ethoxy)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and N,N-dimethyl-2- | |
| | ((1,2,3,4-tetrahydroisoquinolin-7- | |
| | yl)oxy)ethan-1-amine hydrochloride | |
| | (Intermediate YA) | |
| 4.41 | 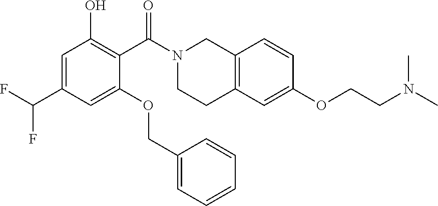 [SMILES: CN(C)CCOc1ccc2c(c1)CCN(C(=O)c1c(O)cc(C(F)F)cc1OCc1ccccc1)C2] | ¹H NMR (DMSO-d6, 400 MHz): δ 2.30 (s, 6H), 2.74 (t, J = 5.6 Hz, 4H), 3.35-3.58 (m, 2H), 4.05 (t, J = 5.6 Hz, 2H), 4.25-4.35 (m, 1H), 4.62- 4.74 (m, 1H), 4.95-5.06 (m, 1H), 5.13 (s, 1H), 6.71-6.84 (m, 4H), 6.95-6.96 (m, 1H), 7.08-7.95 (m, 1H), 7.15-7.31 (m, 5H), 10.2 (s, 1H). LCMS (Method A): 1.382 min, MS ES+ : 497.1 (M + 1). HPLC (Method N): 5.51 min, |
| | (2-(Benzyloxy)-4-s(difluoromethyl)-6- | |
| | hydroxyphenyl)(6-(2- | |
| | (dimethylamino)ethoxy)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and N,N-dimethyl-2- | |
| | ((1,2,3,4-tetrahydroisoquinolin-6- | |
| | yl)oxy)ethan-1-amine hydrochloride | |
| | (Intermediate VF) | |
| 4.42 | 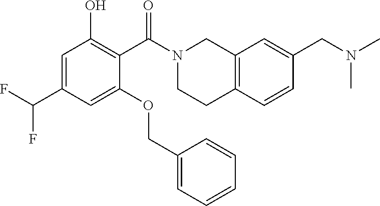 [SMILES: CN(C)Cc1ccc2c(c1)CN(C(=O)c1c(O)cc(C(F)F)cc1OCc1ccccc1)CC2] | ¹H NMR (DMSO-d6, 400 MHz): δ 2.13 (s, 3H), 2.23 (s, 3H), 2.74- 2.78 (m, 2H), 3.37-3.44 (m, 2H), 3.47 (s, 1H), 4.05-4.11 (m, 1H), 4.31-4.41 (m, 1H), 4.72-4.81 (m, 1H), 4.94-5.05 (m, 1H), 5.14 (s, 1H), 6.72 (s, 1H), 6.79-6.82 (m, 1H), 6.95 (s, 1H), 7.05-7.15 (m, 3H), 7.18-7.29 (m, 4H), 10.2 (bs, 1H). ¹H NMR looks complex. LCMS (Method A): 1.440 min, MS ES+ : 467.1 (M + 1). HPLC (Method N): 5.501 min. |
| | (2-(Benzyloxy)-4-(difluoromethyl)-6- | |
| | hydroxyphenyl)(7-((dimethylamino)methyl)- | |
| | 3,4-dihydroisoquinolin-2(1H)-yl)methanone | |
| | Prepared from 2-(benzyloxy)-4- | |
| | (difluoromethyl)-6-hydroxybenzoic acid | |
| | (Intermediate D) and N,N-dimethyl-1- | |
| | (1,2,3,4-tetrahydroisoquinolin-7- | |
| | yl)methanamine (Intermediate YB) | |
| 4.43 | 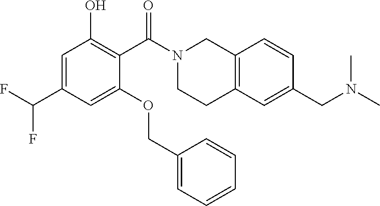 [SMILES: CN(C)Cc1ccc2c(c1)CCN(C(=O)c1c(O)cc(C(F)F)cc1OCc1ccccc1)C2] | ¹H NMR (DMSO-d6, 400 MHz): δ 2.14 (d, J = 3.6 Hz, 6H), 2.74 (s, 2H), 3.36-3.45 (m, 4H), 4.34 (t, J = 12 Hz, 1H), 4.70-4.81 (m, 1H), 4.95- 5.05 (m, 1H), 5.13 (s, 1H), 6.71- 6.81 (m, 2H), 6.94-7.15 (m, 4H), 7.19-7.29 (m, 5H), 10.21 (s, 1H). LCMS (Method A): 1.333 min, MS ES+ : 467 (M + 1). HPLC (Method N): 5.365 min. |
| | (2-(Benzyloxy)-4-(difluoromethyl)-6- | |
| | hydroxyphenyl)(6-((dimethylamino)methyl)- | |
| | 3,4-dihydroisoquinolin-2(1H)-yl)methanone | |
| | Prepared from 2-(benzyloxy)-4- | |
| | (difluoromethyl)-6-hydroxybenzoic acid | |
| | (Intermediate D) and N,N-dimethyl-1- | |
| | (1,2,3,4-tetrahydroisoquinolin-6- | |
| | yl)methanamine (Intermediate YC) | |
| 4.44 | 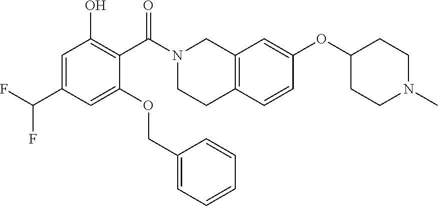 [SMILES: CN1CCC(Oc2ccc3c(c2)CN(C(=O)c2c(O)cc(C(F)F)cc2OCc2ccccc2)CC3)CC1] | ¹H NMR (DMSO-d6, 400 MHz): δ 1.65-1.66 (m, 2H), 1.83-1.96 (m, 2H), 2.18 (s, 3H), 2.24 (s, 1H), 2.33- 2.44 (m, 1H), 2.55-2.67 (m, 3H), 3.35-3.58 (m, 3H), 4.29-4.36 (m, 2H), 4.74 (s, 1H), 5.00-5.07 (m, 2H), 5.14 (s, 1H), 6.67-6.77 (m, 3H), 6.95 (s, 1H), 7.07-7.10 (m, 2H), 7.22-7.27 (m, 5H), 10.20 (bs, 1H). LCMS (Method A): 1.507 min, MS ES+ : 523.03 (M + 1). HPLC (Method N): 5.69 min. |
| | (2-(Benzyloxy)-4-(difluoromethyl)-6- | |
| | hydroxyphenyl)(7-((1-methylpiperidin-4- | |
| | yl)oxy)-3,4-dihydroisoquinolin-2(1H)- | |
| | yl)methanone | |
| | Prepared from 2-(benzyloxy)-4- | |
| | (difluoromethyl)-6-hydroxybenzoic acid | |
| | (Intermediate D) and 7-((1-methylpiperidin-4- | |
| | yl)oxy)-1,2,3,4-tetrahydroisoquinoline | |
| | hydrochloride (Intermediate YD) | |
| 4.45 | 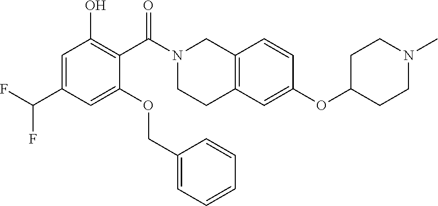 [SMILES: CN1CCC(Oc2ccc3c(c2)CCN(C(=O)c2c(O)cc(C(F)F)cc2OCc2ccccc2)C3)CC1] | ¹H NMR (DMSO-d6, 400 MHz): δ 1.16 (s, 1H), 1.72 (bs, 2H), 1.95 (bs, 2H), 2.44 (s, 3H), 2.70-2.72 (m, 2H), 2.88 (bs, 2H), 3.52-3.58 (m, 1H), 4.25-4.34 (m, 1H), 4.43 (s, 1H), 4.64-4.75 (m, 1H), 4.96-5.06 (m, 1H), 5.13 (s, 2H), 6.71-6.86 (m, 4H), 6.95-6.96 (m, 1H), 7.09-7.16 (m, 1H), 7.18-7.28 (m, 5H), 10.19 (d, J = 16 Hz, 1H) LCMS (Method A): 1.462 min, MS: ES+ 523.1 (M + 1) HPLC (method N): 5.66 min. |
| | (2-(Benzyloxy)-4-(difluoromethyl)-6- | |
| | hydroxyphenyl)(6-((1-methylpiperidin-4- | |
| | yl)oxy)-3,4-dihydroisoquinolin-2(1H)- | |
| | yl)methanone | |
| | Prepared from of 2-(benzyloxy)-4- | |
| | (difluoromethyl)-6-hydroxybenzoic acid | |
| | (Intermediate D) and 6-((1-methylpiperidin-4- | |
| | yl)oxy)-1,2,3,4-tetrahydroisoquinoline | |
| | hydrochloride (Intermediate YE) | |
| 4.46 | 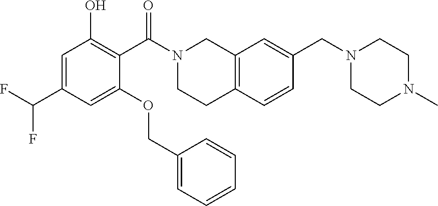 [SMILES: CN1CCN(Cc2ccc3c(c2)CN(C(=O)c2c(O)cc(C(F)F)cc2OCc2ccccc2)CC3)CC1] | ¹H NMR (DMSO-d6, 400 MHz): δ 2.17 (d, J = 16.8 Hz, 3H), 2.29-2.41 (m, 8H), 2.74-2.80 (m, 2H), 3.36- 3.45 (m, 3H), 3.63 (d, J = 12.4 Hz, 1H), 4.01 (s, 1H), 4.35 (d, J = 7.6 Hz, 1H), 4.76 (s, 1H), 4.98-5.13 (m, 2H), 6.75-6.89 (m, 3H), 7.04-7.15 (m, 4H), 7.23-7.29 (m, 4H), 7.29 (s, 1H), 9.86 (s, 1H). LCMS (Method A): 1.400 min, MS: ES+ 522 (M + 1). HPLC (Method C): 5.41 min |
| | (2-(Benzyloxy)-4-(difluoromethyl)-6- | |
| | hydroxyphenyl)(7-((4-methylpiperazin-1- | |
| | yl)methyl)-3,4-dihydroisoquinolin-2(1H)- | |
| | yl)methanone | |
| | Prepared from 2-(benzyloxy)-4- | |
| | (difluoromethyl)-6-hydroxybenzoic acid | |
| | (Intermediate D) and 7-((4-methylpiperazin- | |
| | 1-yl)methyl)-1,2,3,4-tetrahydroisoquinoline | |
| | (Intermediate YF) | |
| 4.47 | 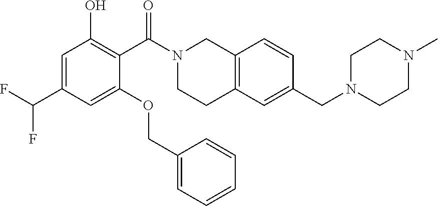 [SMILES: CN1CCN(Cc2ccc3c(c2)CCN(C(=O)c2c(O)cc(C(F)F)cc2OCc2ccccc2)C3)CC1] | ¹H NMR (DMSO-d6, 400 MHz): δ 2.33-2.45 (m, 4H), 2.50-2.60 (m, 3H), 2.67-2.79 (m, 2H), 3.34-3.43 (m, 5H), 4.35-4.37 (m, 1H), 4.74- 4.79 (m, 1H), 4.98-5.05 (m, 1H), 5.14 (s, 1H), 6.55 (s, 1H), 6.78- 6.82 (m, 2H), 6.95-7.29 (m, 8H), 10.18-10.21 (m, 1H). LCMS (Method A): 1.380 min, MS: ES+ 522.1 (M + 1). HPLC (Method N): 5.05 min. |
| | (2-(Benzyloxy)-4-(difluoromethyl)-6- | |
| | hydroxyphenyl)(6-((4-methylpiperazin-1- | |
| | yl)methyl)-3,4-dihydroisoquinolin-2(1H)- | |
| | yl)methanone | |
| | Prepared from 2-(benzyloxy)-4- | |
| | (difluoromethyl)-6-hydroxybenzoic acid | |
| | (Intermediate D) and 6-((4-methylpiperazin- | |
| | 1-yl)methyl)-1,2,3,4-tetrahydroisoquinoline | |
| | hydrochloride (Intermediate YG) | |
| 4.48 | 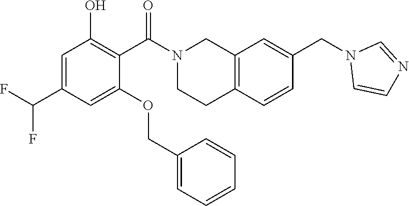 [SMILES: O=C(c1c(O)cc(C(F)F)cc1OCc1ccccc1)N1CCc2ccc(Cn3ccnc3)cc2C1] | ¹H NMR (DMSO-d6, 400 MHz): δ 2.67-2.78 (m, 2H), 3.35-3.59 (m, 2H), 4.28-4.39 (m, 1H), 4.69-4.82 (m, 1H), 4.94-5.12 (m, 4H), 6.32- 6.56 (m, 2H), 6.71 (d, J = 4 Hz, 1H), 6.79 (d, J = 10.8 Hz, 1H), 6.88-7.25 (m, 9H), 7.74-7.83 (m, 1H), 10.22 (s, 1H). LCMS (Method A): 1.440 min, MS: ES+ 490 (M + 1). HPLC (Method N): 5.527 min |
| | (7-((1H-Imidazol-1-yl)methyl)-3,4- | |
| | dihydroisoquinolin-2(1H)-yl)(2-(benzyloxy)- | |
| | 4-(difluoromethyl)-6- | |
| | hydroxyphenyl)methanone | |
| | Prepared from 2-(benzyloxy)-4- | |
| | (difluoromethyl)-6-hydroxybenzoic acid | |
| | (Intermediate D) and 7-((1H-imidazol-1- | |
| | yl)methyl)-1,2,3,4-tetrahydroisoquinoline | |
| | hydrochloride (Intermediate YH) | |
| 4.49 | 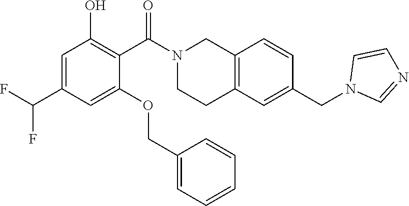 [SMILES: O=C(c1c(O)cc(C(F)F)cc1OCc1ccccc1)N1CCc2cc(Cn3ccnc3)ccc2C1] | ¹H NMR (DMSO-d6, 400 MHz): δ 2.67-2.76 (m, 2H), 3.29 (s, 1H), 3.48-3.55 (m, 1H), 4.29-4.39 (m, 1H), 4.69-4.81 (m, 1H), 4.94-5.04 (m, 1H), 5.13 (s, 3H), 6.71-6.76 (m, 1H), 6.80 (s, 1H), 6.89-6.94 (m, 2H), 7.00-7.13 (m, 3H), 7.16-7.26 (m, 6H), 7.74 (s, 1H), 10.41 (bs, 1H). LCMS (Method A): 1.453 min, MS: ES+ 490 (M + 1). HPLC (Method N): 5.504 min. |
| | (6-((1H-Imidazol-1-yl)methyl)-3,4- | |
| | dihydroisoquinolin-2(1H)-yl)(2-(benzyloxy)- | |
| | 4-(difluoromethyl)-6- | |
| | hydroxyphenyl)methanone | |
| | Prepared from 2-(benzyloxy)-4- | |
| | (difluoromethyl)-6-hydroxybenzoic acid | |
| | (Intermediate D) and 6-((1H-imidazol-1- | |
| | yl)methyl)-1,2,3,4-tetrahydroisoquinoline | |
| | hydrochloride ( Intermediate YI) | |
| 4.50 | 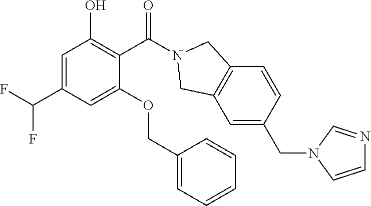 [SMILES: O=C(c1c(O)cc(C(F)F)cc1OCc1ccccc1)N1Cc2ccc(Cn3ccnc3)cc2C1] | ¹H NMR (DMSO-d6, 400 MHz): δ 4.44-4.45 (m, 2H), 4.76 (s, 2H), 5.16-5.17 (m, 3H), 5.20 (s, 1H), 6.74 (s, 1H), 6.81-6.84 (m, 1H), 6.88-6.95 (m, 2H), 7.09-7.21 (m, 3H), 7.22-7.38 (m, 6H), 7.71-7.76 (m, 1H), 10.28 (s, 1H). LCMS (Method A): 1.367 min, MS ES+ : 476.1 (M + 1). HPLC (Method N): 5.437 min. |
| | (5-((1H-Imidazol-1-yl)methyl)isoindolin-2- | |
| | yl)(2-(benzyloxy)-4-(difluoromethyl)-6- | |
| | hydroxyphenyl)methanone | |
| | Prepared from 2-(benzyloxy)-4- | |
| | (difluoromethyl)-6-hydroxybenzoic acid | |
| | (Intermediate D) and 5-((1H-imidazol-1- | |
| | yl)methyl)isoindoline hydrochloride | |
| | (Intermediate YJ) | |
| 4.51 | 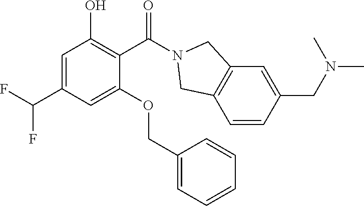 [SMILES: CN(C)Cc1ccc2c(c1)CN(C(=O)c1c(O)cc(C(F)F)cc1OCc1ccccc1)C2] | ¹H NMR (DMSO-d6, 400 MHz): δ 2.14-2.18 (m, 6H), 3.44 (s, 2H), 4.46 (d, J = 8.42 Hz, 2H), 4.77 (s, 2H), 5.17 (s, 2H), 6.75 (s, 1H), 6.82- 7.10 (m, 2H), 7.10-7.20 (m, 2H), 7.23-7.35 (m, 6H), 10.28 (s, 1H). LCMS (Method A): 1.322 min, MS: ES+ 453.17 (M + 1). HPLC (Method N): 5.281 min. |
| | (2-(Benzyloxy)-4-(difluoromethyl)-6- | |
| | hydroxyphenyl)(5- | |
| | ((dimethylamino)methyl)isoindolin-2- | |
| | yl)methanone | |
| | Prepared from 2-(benzyloxy)-4- | |
| | (difluoromethyl)-6-hydroxybenzoic acid | |
| | (Intermediate D) and 1-(isoindolin-5-yl)-N,N- | |
| | dimethylmethanamine HCl (Intermediate R) | |
| 4.52 | 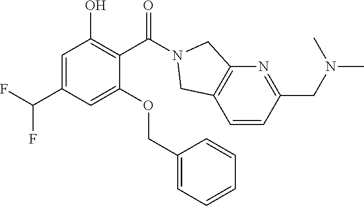 [SMILES: CN(C)Cc1ccc2c(n1)CN(C(=O)c1c(O)cc(C(F)F)cc1OCc1ccccc1)C2] | ¹H NMR (DMSO-d6, 400 MHz): δ 2.17-2.21 (m, 6H), 3.49-3.55 (m, 2H), 4.38-4.49 (m, 2H), 4.72-4.81 (m, 2H), 5.17 (s, 2H), 6.77 (s, 1H), 6.82-6.86 (m, 1H), 6.96-7.10 (m, 1H), 7.22-7.38 (m, 6H), 7.65-7.82 (m, 1H), 10.38 (s, 1H). LCMS (Method X): 4.390 min, MS: ES+ 454.4 (M + 1). HPLC (Method N): 4.94 min. |
| | (2-(Benzyloxy)-4-(difluoromethyl)-6- | |
| | hydroxyphenyl)(2-((dimethylamino)methyl)- | |
| | 5,7-dihydro-6H-pyrrolo[3,4-b]pyridin-6- | |
| | yl)methanone | |
| | Prepared from 2-(benzyloxy)-4- | |
| | (difluoromethyl)-6-hydroxybenzoic acid | |
| | (Intermediate D) and 1-(6,7-dihydro-5H- | |
| | pyrrolo[3,4-b] pyridin-2-yl)-N,N- | |
| | dimethylmethanamine hydrochloride | |
| | (Intermediate T) | |
| 4.53 | 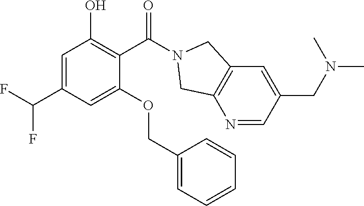 [SMILES: CN(C)Cc1cnc2c(c1)CN(C(=O)c1c(O)cc(C(F)F)cc1OCc1ccccc1)C2] | ¹H NMR (DMSO-d6, 400 MHz): δ 2.13-2.16 (m, 6H), 3.40-3.44 (m, 2H), 4.39-4.51 (m, 2H), 4.73-4.82 (m, 2H), 5.18 (s, 2H), 6.75 (s, 1H), 6.82-7.10 (m, 2H), 7.20-7.32 (m, 5H), 7.59-7.76 (m, 1H), 8.34-8.38 (m, 1H), 10.37 (bs, 1H). LCMS (Method A): 1.286 min, MS: ES+ 454 (M + 1). HPLC (Method N): 5.041 min |
| | (2-(Benzyloxy)-4-(difluoromethyl)-6- | |
| | hydroxyphenyl)(3-((dimethylamino)methyl)- | |
| | 5,7-dihydro-6H-pyrrolo[3,4-b]pyridin-6- | |
| | yl)methanone | |
| | Prepared from 2-(benzyloxy)-4- | |
| | (difluoromethyl)-6-hydroxybenzoic acid | |
| | (Intermediate D) and 1-(6,7-dihydro-5H- | |
| | pyrrolo[3,4-b]pyridin-3-yl)-N,N- | |
| | dimethylmethanamine hydrochloride | |
| | (Intermediate YK) | |
| 4.54 | 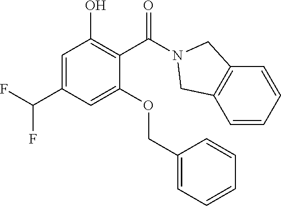 [SMILES: O=C(c1c(O)cc(C(F)F)cc1OCc1ccccc1)N1Cc2ccccc2C1] | ¹H NMR (DMSO-d6, 400 MHz): δ 4.46-4.48 (bs, 2H), 4.79 (s, 2H), 5.17 (s, 2H), 6.75 (s, 1H), 6.81- 7.09 (m, 2H), 7.23-7.33 (m, 7H), 7.39 (s, 1H), 10.28 (s, 1H). LCMS (Method N): 1.730 min, MS: ES+ 396.0 (M + 1). HPLC (Method G): 7.91 min. |
| | (2-(Benzyloxy)-4-(difluoromethyl)-6- | |
| | hydroxyphenyl)(isoindolin-2-yl)methanone | |
| | Prepared from 2-(benzyloxy)-4- | |
| | (difluoromethyl)-6-hydroxybenzoic acid | |
| | (Intermediate D) and Isoindoline | |
| | hydrochloride (CAS 32372-82-0) | |
| 4.55 | 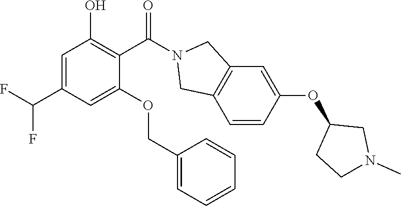 [SMILES: CN1CC[C@@H](Oc2ccc3c(c2)CN(C(=O)c2c(O)cc(C(F)F)cc2OCc2ccccc2)C3)C1] | ¹H NMR (DMSO-d6, 400 MHz): δ 1.71-1.74 (m, 1H), 2.22-2.36 (m, 4H), 2.55-2.79 (m, 3H), 4.39 (t, J = 14.8 Hz, 2H), 4.69-4.84 (m, 3H), 5.17 (s, 2H), 6.74 (s, 1H), 6.77- 6.84 (m, 3H), 6.91-6.95 (m, 1H), 7.09-7.14 (m, 1H), 7.24-7.32 (m, 5H), 10.29 (s, 1H). LCMS (Method N): 1.352 min, MS ES+ : 495.1 (M + 1). HPLC (Method G): 4.86 min. |
| | (R)-(2-(Benzyloxy)-4-(difluoromethyl)-6- | |
| | hydroxyphenyl)(5-((1-methylpyrrolidin-3- | |
| | yl)oxy)isoindolin-2-yl)methanone | |
| | Prepared from 2-(benzyloxy)-4- | |
| | (difluoromethyl)-6-hydroxybenzoic acid | |
| | (Intermediate D) and (R)-5-((1- | |
| | methylpyrrolidin-3-yl)oxy)isoindoline | |
| | hydrochloride (Intermediate VA) | |
| 4.56 | 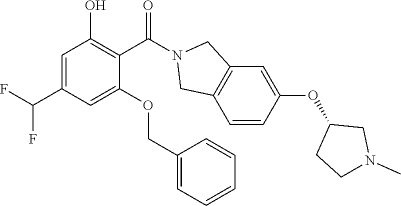 [SMILES: CN1CC[C@H](Oc2ccc3c(c2)CN(C(=O)c2c(O)cc(C(F)F)cc2OCc2ccccc2)C3)C1] | ¹H NMR (DMSO-d6, 400 MHz): δ 1.71-1.76 (m, 1H), 2.22-2.38 (m, 5H), 2.50-2.79 (m, 3H), 4.37-4.42 (m, 2H), 4.69-4.84 (m, 3H), 5.16 (s, 2H), 6.74-6.83 (m, 4H), 6.91-6.95 (m, 1H), 7.08-7.14 (m, 1H), 7.24- 7.32 (m, 5H), 10.30 (s, 1H). LCMS (Method P): 4.881 min, MS: ES+ 495.2 (M + 1). HPLC (Method I): 7.38 min. |
| | (S)-(2-(Benzyloxy)-4-(difluoromethyl)-6- | |
| | hydroxyphenyl)(5-((1-methylpyrrolidin-3- | |
| | yl)oxy)isoindolin-2-yl)methanone | |
| | Prepared from 2-(benzyloxy)-4- | |
| | (difluoromethyl)-6-hydroxybenzoic acid | |
| | (Intermediate D) and (S)-5-((1- | |
| | methylpyrrolidin-3-yl)oxy)isoindoline | |
| | hydrochloride (Intermediate VB) | |
| 4.57 | 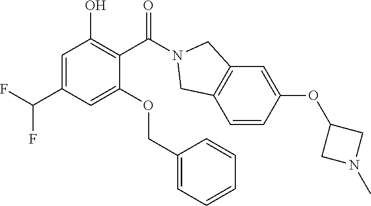 [SMILES: CN1CC(Oc2ccc3c(c2)CN(C(=O)c2c(O)cc(C(F)F)cc2OCc2ccccc2)C3)C1] | ¹H NMR (DMSO-d6, 400 MHz): δ 2.33 (d, J = 9.2 Hz, 3H), 2.94-3.02 (m, 2H), 3.73-3.80 (m, 2H), 4.37- 4.44 (m, 2H), 4.66-4.75 (m, 3H), 5.18 (s, 2H), 6.75-6.79 (m, 2H), 6.82-6.85 (m, 2H), 6.95-6.96 (m, 1H), 7.10-7.17 (m, 1H), 7.26-7.33 (m, 5H), 10.31-10.32 (m, 1H). LCMS (Method N): 1.349 min, MS ES+ : 481.0 (M + 1). HPLC (Method G): 4.798 min |
| | (2-(Benzyloxy)-4-(difluoromethyl)-6- | |
| | hydroxyphenyl)(5-((1-methylazetidin-3- | |
| | yl)oxy)isoindolin-2-yl)methanone | |
| | Prepared from 2-(benzyloxy)-4- | |
| | (difluoromethyl)-6-hydroxybenzoic acid | |
| | (Intermediate D) and 5-((1-methylazetidin-3- | |
| | yl)oxy)isoindoline TFA salt (Intermediate VC) | |
| 4.58 | 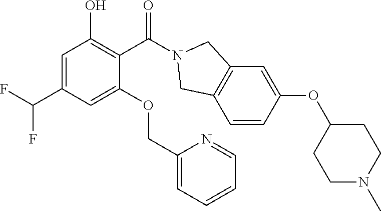 [SMILES: CN1CCC(Oc2ccc3c(c2)CN(C(=O)c2c(O)cc(C(F)F)cc2OCc2ccccn2)C3)CC1] | ¹H NMR (DMSO-d6, 400 MHz): δ 1.60-1.66 (m, 2H), 1.90 (m, 2H), 2.08-2.17 (m, 5H), 2.59 (d, J = 6 Hz, 2H), 4.23-4.36 (m, 1H), 4.45 (d, J = 15.6 Hz, 2H), 4.73 (d, J = 14.8 Hz, 2H), 5.23 (s, 2H), 6.79 (d, J = 1.6 Hz, 1H), 6.81-6.85 (m, 3H), 6.93-7.29 (m, 3H), 7.36 (d, J = 7.6 Hz, 1H), 7.69-7.74 (m, 1H), 8.49-8.51 (m, 1H), 10.33 (s, 1H). LCMS (Method N): 1.17 min, MS: ES+ 510.0 (M + 1). HPLC (Method I): 5.64 min |
| | (4-(Difluoromethyl)-2-hydroxy-6-(pyridin-2- | |
| | ylmethoxy)phenyl)(5-((1-methylpiperidin-4- | |
| | yl)oxy)isoindolin-2-yl)methanone | |
| | Prepared from 4-(difluoromethyl)-2-hydroxy- | |
| | 6-(pyridin-2-ylmethoxy)benzoic acid | |
| | (Intermediate DB) and 5-((1-methylpiperidin- | |
| | 4-yl)oxy)isoindoline hydrochloride | |
| | (Intermediate V) | |
| 4.59 | 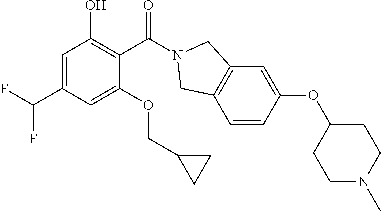 [SMILES: CN1CCC(Oc2ccc3c(c2)CN(C(=O)c2c(O)cc(C(F)F)cc2OCC2CC2)C3)CC1] | ¹H NMR (DMSO-d6, 400 MHz): δ 0.22 (d, J = 3.2 Hz, 2H), 0.43 (d, J = 6.8 Hz, 2H), 1.03 (d, J = 4 Hz, 1H), 1.10 (m, 2H), 1.55-1.65 (m, 2H), 1.90 (bs, 2H), 2.09-2.17 (m, 4H), 2.59 (d, J = 4 Hz, 2H), 3.88 (d, J = 6.8 Hz, 2H), 4.34-4.41 (m, 2H), 4.70 (m, 2H), 6.72 (d, J = 7.6 Hz, 2H), 6.80-6.99 (m, 3H), 7.13-7.27 (m, 1H), 10.17 (s, 1H). LCMS (Method V): 7.63 min, MS: ES+ 473 (M + 1). HPLC (Method I): 7.12 min. |
| | (2-(Cyclopropylmethoxy)-4-(difluoromethyl)- | |
| | 6-hydroxyphenyl)(5-((1-methylpiperidin-4- | |
| | yl)oxy)isoindolin-2-yl)methanone | |
| | Prepared from 2-(cyclopropylmethoxy)-4- | |
| | (difluoromethyl)-6-hydroxybenzoic acid | |
| | (Intermediate YL) and 5-((1-methylpiperidin- | |
| | 4-yl)oxy)isoindoline hydrochloride | |
| | (Intermediate V) | |
| 4.60 | 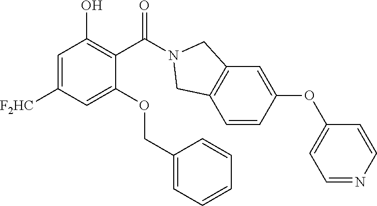 [SMILES: O=C(c1c(O)cc(C(F)F)cc1OCc1ccccc1)N1Cc2ccc(Oc3ccncc3)cc2C1] | ¹H NMR (DMSO-d6, 400 MHz): δ 1.48-1.51 (m, 2H), 4.82 (s, 2H), 5.20 (s, 2H), 6.76-6.97 (m, 4H), 7.10-7.15 (m, 2H), 7.25-7.51 (m, 7H), 8.44-8.48 (m, 2H), 10.33 (s, 1H). LCMS (Method N): 1.373 min, MS ES+ : 489.0 (M + 1). HPLC (Method G): 4.88 min. |
| | (2-(Benzyloxy)-4-(difluoromethyl)-6- | |
| | hydroxyphenyl)(5-(pyridin-4-yloxy)isoindolin- | |
| | 2-yl)methanone | |
| | Prepared from 2-(benzyloxy)-4- | |
| | (difluoromethyl)-6-hydroxybenzoic | |
| | acid(Intermediate D) and 5-(pyridin-4- | |
| | yloxy)isoindoline hydrochloride | |
| | (Intermediate YM) | |
| 4.61 | 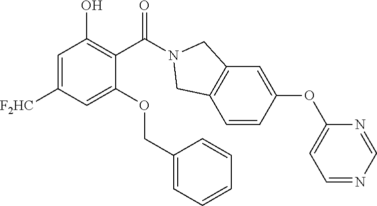 [SMILES: O=C(c1c(O)cc(C(F)F)cc1OCc1ccccc1)N1Cc2ccc(Oc3ccncn3)cc2C1] | ¹H NMR (DMSO-d6, 400 MHz): δ 4.48(m, 2H), 4.80 (s, 2H), 5.18 (s, 1H ), 6.74-6.96 (m, 3H), 7.12- 7.17 (m, 2H), 7.24-7.47 (m, 7H), 8.66-8.69 (m, 1H), 8.75 (d, J = 9.6 Hz, 1H). LCMS (Method N): 1.56min, MS: ES+ 489.9 (M + 1). HPLC (Method G): 6.69 min. |
| | (2-(Cyclopropylmethoxy)-4-(difluoromethyl)- | |
| | 6-hydroxyphenyl)(5-((1-methylpiperidin-4- | |
| | yl)oxy)isoindolin-2-yl)methanone | |
| | Prepared from 2-(benzyloxy)-4- | |
| | (difluoromethyl)-6-hydroxybenzoic acid | |
| | (Intermediate D) and 5-(pyrimidin-4- | |
| | yloxy)isoindoline (Intermediate YN) | |
| 4.62 | 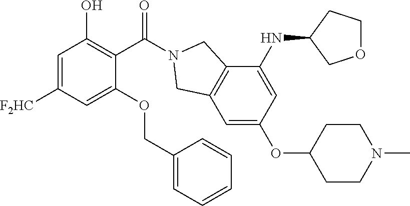 [SMILES: CN1CCC(Oc2cc3c(c(N[C@H]4CCOC4)c2)CN(C(=O)c2c(O)cc(C(F)F)cc2OCc2ccccc2)C3)CC1] | ¹H NMR (DMSO-d6, 400 MHz): δ 1.62-1.67 (m, 3H), 1.87-2.09 (m, 3H), 2.05-2.2 (m, 1H), 2.26 (d, J = 14.8 Hz, 3H), 2.33 (s, 1H), 2.68- 2.71 (m, 1H), 3.48-3.51 (m, 1H), 3.57-3.75 (m, 2H), 3.81-3.83 (m, 2H), 3. 85-3.92 (m, 1H), 4.23-4.34 (m, 3H), 4.54 (s, 1H), 4.64 (s, 1H), 5.17 (s, 2H), 5.41-5.52 (m, 1H, D₂O exchangeable), 5.98 (d, J = 10.4 Hz, 1H), 6.13-6.26 (m, 1H), 6.74-6.83 (m, 2H), 6.95-7.09 (m, 1H), 7.24- 7.35 (m, 5H), 10.3 (s, 1H). LCMS (Method V): 8.146 min, MS |
| | (S)-(2-(Benzyloxy)-4-(difluoromethyl)-6- | ES+ : 594.5 (M + 1). |
| | hydroxyphenyl)(6-((1-methylpiperidin-4- | HPLC (Method M): 8.146 min. |
| | yl)oxy)-4-((tetrahydrofuran-3- | |
| | yl)amino)isoindolin-2-yl)methanone | |
| | Prepared from 2-(benzyloxy)-4- | |
| | (difluoromethyl)-6-hydroxybenzoic acid | |
| | (Intermediate D) and (S)-6-((1- | |
| | methylpiperidin-4-yl)oxy)-N- | |
| | (tetrahydrofuran-3-yl)isoindolin-4-amine | |
| | hydrochloride (Intermediate YO) | |
| 4.63 | 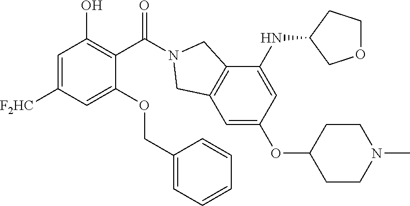 [SMILES: CN1CCC(Oc2cc3c(c(N[C@@H]4CCOC4)c2)CN(C(=O)c2c(O)cc(C(F)F)cc2OCc2ccccc2)C3)CC1] | HT ¹H NMR (DMSO-d6, 400 MHz, 349K): δ 1.60-1.85 (m, 6H), 1.94- 1.99 (m, 7H), 2.22 (d, J = 12.8 Hz, 4H), 2.68 (s, 2H), 3.49-3.62 (m, 1H), 3.67-3.80 (m, 2H), 3.81-3.93 (m, 1H), 3.98-4.1 (m, 1H), 4.15- 4.30 (m, 1H), 4.26-4.33 (m, 2H), 4.57 (s, 1H), 4.65-4.70 (m, 1H), 4.80-4.90 (m, 2H), 5.16 (s, 2H), 5.25-5.30 (m, 1H, D₂O exchangeable), 6.03 (d, J = 12.4 Hz, 1H), 6.12-6.26 (m, 1H), 6.76-6.90 (m, 3H), 7.25-7.36 (m, 5H), 9.95 (s, 1H). |
| | (R)-(2-(Benzyloxy)-4-(difluoromethyl)-6- | LCMS (Method V): 8.279 min, MS |
| | hydroxyphenyl)(6-((1-methylpiperidin-4- | ES+ : 593.3 (M-1). |
| | yl)oxy)-4-((tetrahydrofuran-3- | HPLC (Method M): 8.146 min. |
| | yl)amino)isoindolin-2-yl)methanone | |
| | Prepared from 2-(benzyloxy)-4- | |
| | (difluoromethyl)-6-hydroxybenzoic acid | |
| | (Intermediate D) and (R)-6-((1- | |
| | methylpiperidin-4-yl)oxy)-N- | |
| | (tetrahydrofuran-3-yl)isoindolin-4-amine | |
| | hydrochloride (Intermediate YP) | |
| 4.64 | 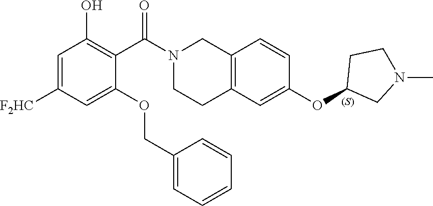 | ¹H NMR (DMSO-d6, 400 MHz): δ 1.71-1.77 (m, 1H), 2.23-2.26 (m, 4H), 2.31-2.34 (m, 1H), 2.53-2.56 (m, 1H), 2.62-2.77 (m, 4H), 3.35- 3.40 (m, 1H), 3.60 (m, 1H), 4.25- 4.39 (m, 1H), 4.69 (d, J = 8.4 Hz, 1H), 4.80-4.81 (m, 1H), 4.99-5.03 (m, 1H), 5.13 (s, 1H), 6.61-6.77 (m, 3H), 6.81 (s, br, 1H), 6.92-6.94 (m, 1H), 7.08-7.15 (dd, J = 6 Hz, 8.4 Hz, 2H), 7.18-7.28 (m, 4H), 10.12 (s, 1H). LCMS (Method N): 1.345 min, MS ES+ : 509.2 (M + 1). |
| | (S)-(2-(Benzyloxy)-4-(difluoromethyl)-6- | HPLC (Method G): 4.067 min. |
| | hydroxyphenyl)(6-((1-methylpyrrolidin-3- | |
| | yl)oxy)-3,4-dihydroisoquinolin-2(1H) | |
| | yl)methanone | |
| | Prepared from 2-(benzyloxy)-4- | |
| | (difluoromethyl)-6-hydroxybenzoic acid | |
| | (Intermediate D) and (S)-6-((1- | |
| | methylpyrrolidin-3-yl)oxy)-1,2,3,4- | |
| | tetrahydroisoquinoline hydrochloride | |
| | (Intermediate YQ) | |
| 4.65 | 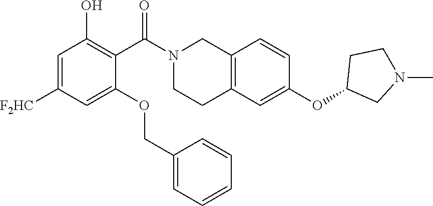 [SMILES: CN1CC[C@@H](Oc2ccc3c(c2)CCN(C(=O)c2c(O)cc(C(F)F)cc2OCc2ccccc2)C3)C1] | ¹H NMR (DMSO-d6, 400 MHz): δ 1.73-1.76 (m, 1H), 2.23-2.34 (m, 5H), 2.56-2.69 (m, 1H), 2.70-2.77 (m, 3H), 3.40 (s, 1H), 3.53-4.20 (m, 2H), 4.28-4.35 (m, 1H), 4.58-4.81 (m, 2H), 4.99-5.03 (m, 1H), 5.13 (s, 1H), 6.61-6.76 (m, 3H), 6.77-6.81 (m, 1H), 6.92-6.94 (m, 1H), 7.08- 7.10 (m, 1H), 7.14 (d, J = 8.4 Hz, 1H), 7.18-7.28 (m, 4H), 10.23 (s, 1H). LCMS (Method A): 1.339 min, MS ES+ : 509.2 (M + 1). HPLC (Method H): 4.046 min. |
| | (R)-(2-(Benzyloxy)-4-(difluoromethyl)-6- | |
| | hydroxyphenyl)(6-((1-methylpyrrolidin-3- | |
| | yl)oxy)-3,4-dihydroisoquinolin-2(1H) | |
| | yl)methanone | |
| | Prepared from 2-(benzyloxy)-4- | |
| | (difluoromethyl)-6-hydroxybenzoic acid | |
| | (Intermediate D) and (R)-6-((1- | |
| | methylpyrrolidin-3-yl)oxy)-1,2,3,4- | |
| | tetrahydroisoquinoline hydrochloride | |
| | (Intermediate YR) | |
| 4.66 | 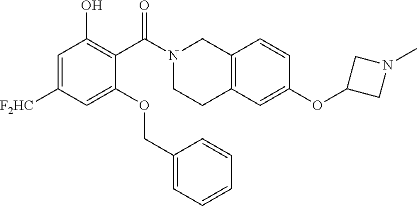 [SMILES: CN1CC(Oc2ccc3c(c2)CCN(C(=O)c2c(O)cc(C(F)F)cc2OCc2ccccc2)C3)C1] | ¹H NMR (DMSO-d6, 400 MHz): δ 2.29 (m, 3H), 2.68-2.78 (m, 2H), 2.92-2.96 (m, 2H), 3.56-4.06 (m, 3H), 4.30 (d, J = 4.8, Hz, 1H), 4.69- 4.73 (m, 1H), 5.01 (q, J = 12 Hz, 1H), 5.14 (s, 1H), 6.34-6.65 (m, 3H), 6.70-6.82 (m, 3H), 6.94-6.95 (m, 1H), 7.08-7.10 (m, 1H), 7.15-7.29 (m, 4H), 9.55 (bs, 1H). LCMS (Method N): 1.335 min, MS: ES+ 495.1 (M + 1). HPLC (Method G): 3.995 min. |
| | (2-(Benzyloxy)-4-(difluoromethyl)-6- | |
| | hydroxyphenyl)(6-((1-methylazetidin-3- | |
| | yl)oxy)-3,4-dihydroisoquinolin-2(1H)- | |
| | yl)methanone | |
| | Prepared from 2-(benzyloxy)-4- | |
| | (difluoromethyl)-6-hydroxybenzoic acid | |
| | (Intermediate D) and 6-((1-methylazetidin-3- | |
| | yl)oxy)-1,2,3,4-tetrahydroisoquinoline TFA | |
| | salt (Intermediate YS) | |
| 4.67 | 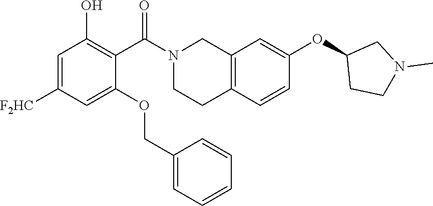 [SMILES: CN1CC[C@@H](Oc2ccc3c(c2)CN(C(=O)c2c(O)cc(C(F)F)cc2OCc2ccccc2)CC3)C1] | ¹H NMR (DMSO-d6, 400 MHz): δ 1.74-1.76 (m, 1H), 2.21-2.33 (m, 4H), 2.72 (bs, 3H), 3.37-3.55 (m, 2H), 4.73-4.76 (m, 1H), 4.73-4.99 (m, 2H), 5.00-5.14 (m, 2H), 6.55- 6.82 (m, 4H), 6.94-7.08 (m, 3H), 7.19-7.27 (m, 4H), 10.20 (bs, 1H). LCMS (Method N): 1.373 min, MS ES+ : 509 (M + 1). HPLC (Method G): 4.68 min. |
| | (R)-(2-(Benzyloxy)-4-(difluoromethyl)-6- | |
| | hydroxyphenyl)(7-((1-methylpyrrolidin-3- | |
| | yl)oxy)-3,4-dihydroisoquinolin-2(1H)- | |
| | yl)methanone | |
| | Prepared from 2-(benzyloxy)-4- | |
| | (difluoromethyl)-6-hydroxybenzoic acid | |
| | (Intermediate D) and (R)-7-((1- | |
| | methylpyrrolidin-3-yl)oxy)-1,2,3,4- | |
| | tetrahydroisoquinoline hydrochloride | |
| | (Intermediate YT) | |
| 4.68 | 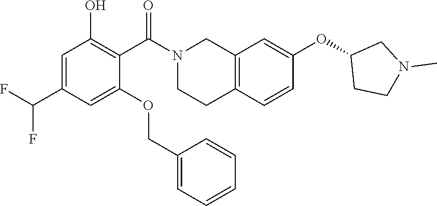 [SMILES: CN1CC[C@H](Oc2ccc3c(c2)CN(C(=O)c2c(O)cc(C(F)F)cc2OCc2ccccc2)CC3)C1] | ¹H NMR (DMSO-d6, 400 MHz): δ 2.20 (s, 1H), 2.26 (s, 2H), 2.45- 2.47 (m, 1H), 2.50-2.59 (m, 3H), 2.62-2.71 (m, 3H), 2.76-2.80 (m, 1H), 3.30 (m, 1H), 3.50-3.56 (m, 1H), 4.33-4.34 (m, 1H), 4.73-4.77 (m, 1H), 4.82-4.99 (m, 1H), 5.03- 5.06 (m, 1H), 5.13 (s, 1H), 6.59- 6.71 (m, 2H), 6.78-6.81 (m, 2H), 6.94-7.011 (m, 1H), 7.07-7.29 (m, 6H), 10.25 (s, 1H). LCMS (Method N): 1.39 min, MS: ES+ 509.0 (M + 1). HPLC (Method I): 7.65 min. |
| | (S)-(2-(Benzyloxy)-4-(difluoromethyl)-6- | |
| | hydroxyphenyl)(7-((1-methylpyrrolidin-3- | |
| | yl)oxy)-3,4-dihydroisoquinolin-2(1H)- | |
| | yl)methanone | |
| | Prepared from 2-(benzyloxy)-4- | |
| | (difluoromethyl)-6-hydroxybenzoic acid | |
| | (Intermediate D) and (S)-7-((1- | |
| | methylpyrrolidin-3-yl) oxy)-1,2,3,4- | |
| | tetrahydroisoquinoline hydrochloride | |
| | (Intermediate YU) | |
| 4.69 | 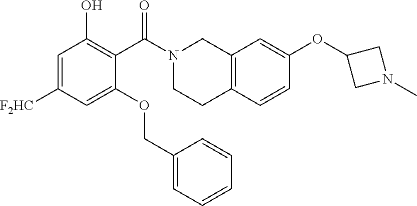 [SMILES: CN1CC(Oc2ccc3c(c2)CN(C(=O)c2c(O)cc(C(F)F)cc2OCc2ccccc2)CC3)C1] | ¹H NMR (DMSO-d6, 400 MHz): δ 2.25-2.34 (m, 3H), 2.67-2.88 (m, 2H), 2.86-2.97 (m, 2H), 3.51-3.62 (m, 1H), 3.67-3.78 (m, 2H), 4.04 (m, 1H), 4.34-4.35 (m, 1H), 4.63- 4.65 (m, 1H), 4.72-4.73 (m, 1H), 4.97-5.00 (m, 1H), 5.14 (s, 1H), 6.55-6.82 (m, 4H), 6.95-7.09 (m, 3H), 7.19-7.30 (m, 4H), 10.20 (d, J = 15.6 Hz, 1H). LCMS (Method N): 1.36 min, MS: ES+ 495.1 (M + 1). HPLC (Method G): 4.64 min. |
| | (2-(Benzyloxy)-4-(difluoromethyl)-6- | |
| | hydroxyphenyl)(7-((1-methylazetidin-3- | |
| | yl)oxy)-3,4-dihydroisoquinolin-2(1H)- | |
| | yl)methanone | |
| | Prepared from 2-(benzyloxy)-4- | |
| | (difluoromethyl)-6-hydroxybenzoic acid | |
| | (Intermediate D) and 7-((1-methylazetidin-3- | |
| | yl)oxy)-1,2,3,4-tetrahydroisoquinoline | |
| | hydrochloride (Intermediate YV) | |
| 4.70 | 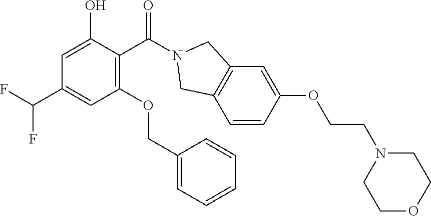 [SMILES: O=C(c1c(O)cc(C(F)F)cc1OCc1ccccc1)N1Cc2ccc(OCCN3CCOCC3)cc2C1] | ¹H NMR (DMSO-d6, 400 MHz): δ 2.26 (s, 6H), 2.46-2.67 (m, 4H), 3.54-3.59 (m, 2H), 4.01-4.09 (m, 2H), 4.38 (d, J = 12.4 Hz, 2H), 4.71 (s, 1H), 5.19 (s, 2H), 6.58-6.94 (m, 4H), 7.08-7.30 (m, 7H), 10.29 (bs, 1H). LCMS (Method N): 1.274 min, MS: ES+ 472.1 (M + 1). HPLC (Method G): 4.33 min. |
| | (2-(Benzyloxy)-4-(difluoro methyl)-6- | |
| | hydroxyphenyl) (5-(2-morpholinoethoxy) | |
| | isoindolin-2-yl) methanone | |
| | Prepared from 2-(benzyloxy)-4- | |
| | (difluoromethyl)-6-hydroxybenzoic acid | |
| | (Intermediate D) and 4-(2-(isoindolin-5- | |
| | yloxy) ethyl) morpholine hydrochloride | |
| | (Intermediate YW) | |
| 4.71 | 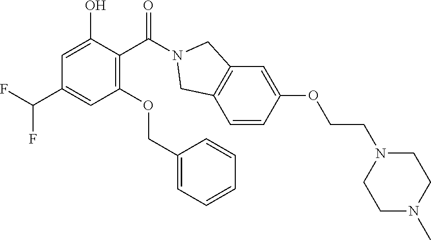 [SMILES: CN1CCN(CCOc2ccc3c(c2)CN(C(=O)c2c(O)cc(C(F)F)cc2OCc2ccccc2)C3)CC1] | ¹H NMR, D₂O exchange (DMSO-d6, 400 MHz): δ 2.20 (d, J = 8.0 Hz, 4H), 2.40-2.71 (m, 7H), 4.01-4.09 (m, 2H), 4.38-4.44 (m, 2H) 4.72 (d, J = 14.4 Hz, 2H), 5.18 (s, 2H), 6.75- 6.67 (m, 4H), 6.91-6.99 (m, 2H), 7.00-7.31 (m, 5H), 10.30 (bs, 1H). LCMS (Method N): 1.347 min, MS: ES+ 538.0 (M + 1). HPLC (Method I): 6.57 min. |
| | (2-(Benzyloxy)-4-(difluoromethyl)-6- | |
| | hydroxyphenyl) (5-(2-(4-methylpiperazin-1- | |
| | yl) ethoxy) isoindolin-2-yl) methanone | |
| | Prepared from 2-(benzyloxy)-4- | |
| | (difluoromethyl)-6-hydroxybenzoic acid | |
| | (Intermediate D) and 5-(2-(4- | |
| | methylpiperazin-1-yl) ethoxy) isoindoline | |
| | hydrochloride (Intermediate YX) | |
| 4.72 | 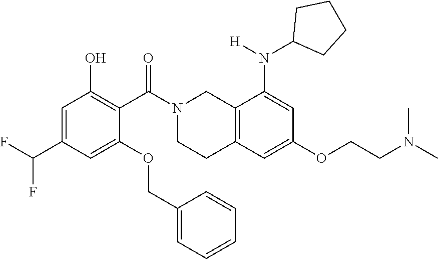 [SMILES: CN(C)CCOc1cc2c(c(NC3CCCC3)c1)CN(C(=O)c1c(O)cc(C(F)F)cc1OCc1ccccc1)CC2] | ¹H NMR (DMSO-d6, 400 MHz): δ 1.70 (bs, 2H), 1.95 (s, 6H), 2.54- 2.68 (m, 4H), 3.35 (s, 2H), 3.72-3.74 (m, 1H), 3.94-3.98 (m, 2H),4.34- 4.57 (m, 2H), 4.76 (d, J = 4 Hz, 1H), 5.14 (s, 2H), 5.96-6.00 (m, 1H) 6.03-6.07 (m, 1H), 6.70-7.08 (m, 5H), 7.21-7.35 (m, 5H), 10.18 (bs, 1H). LCMS (Method N): 1.497 min, MS: ES+ 580.1 (M + 1). HPLC (Method G): 4.831 min. |
| | (2-(Benzyloxy)-4-(difluoromethyl)-6- | |
| | hydroxyphenyl)(8-(cyclopentylamino)-6-(2- | |
| | (dimethylamino)ethoxy)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and N-cyclopentyl-6-(2- | |
| | (dimethyl amino) ethoxy)-1,2,3,4- | |
| | tetrahydroisoquinolin-8-amine hydrochloride | |
| | (Intermediate YY) | |
| 4.73 | 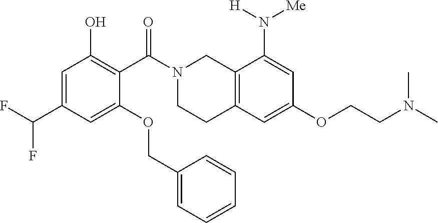 [SMILES: CNc1cc(OCCN(C)C)cc2c1CN(C(=O)c1c(O)cc(C(F)F)cc1OCc1ccccc1)CC2] | ¹H NMR (DMSO-d6, 400 MHz): δ 2.24 (s, 6H), 2.60-2.64 (m, 3H), 2.67 (m, 2H), 2.75 (d, J = 4.8 Hz, 2H), 3.34-3.47 (m, 2H), 3.50-3.53 (m, 1H), 3.63 (m, 1H), 3.98-4.05 (m, 2H), 4.10 (s, 1H), 4.37-4.41 (m, 1H), 4.51-4.56 (m, 1H), 5.00-5.06 (m, 1H), 5.14 (s, 1H), 5.89-6.05 (m, 2H), 6.69-6.78 (m, 3H), 6.81 (s, br, 1H), 7.08-7.15 (m, 1H), 7.22- 7.30 (m, 5H), 9.85 (s, 1H). LCMS (Method N): 1.375 min, MS: ES+ 526.13 (M + 1). HPLC (Method G): 3.93 min. |
| | (2-(Benzyloxy)-4-(difluoromethyl)-6- | |
| | hydroxyphenyl)(6-(2- | |
| | (dimethylamino)ethoxy)-8-(methylamino)- | |
| | 3,4-dihydroisoquinolin-2(1H)-yl)methanone | |
| | Prepared from 2-(benzyloxy)-4- | |
| | (difluoromethyl)-6-hydroxybenzoic acid | |
| | (Intermediate D) and 6-(2- | |
| | (dimethylamino)ethoxy)-N-methyl-1,2,3,4- | |
| | tetrahydroisoquinolin-8-amine hydrochloride | |
| | (Intermediate YZ) | |
| 4.74 | 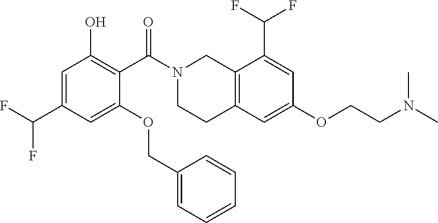 [SMILES: CN(C)CCOc1cc2c(c(C(F)F)c1)CN(C(=O)c1c(O)cc(C(F)F)cc1OCc1ccccc1)CC2] | ¹H NMR (DMSO-d6, 400 MHz): δ 2.24 (s, 6H), 2.64-2.66 (m, 2H), 2.78-2.80 (m, 2H), 4.04-4.11 (m, 2H), 4.44-4.52 (m, 1H), 4.78-4.87 (m, 2H), 4.99-5.15 (m, 2H), 6.71- 6.83 (m, 3H), 6.89-7.09 (m, 4H), 7.17-7.30 (m, 4H), 10.24 (s, 1H). LCMS (Method N): 1.383 min, MS: ES+ 547.2 (M + 1). HPLC (Method G): 4.49 min. |
| | (2-(Benzyloxy)-4-(difluoromethyl)-6- | |
| | hydroxyphenyl)(8-(difluoromethyl)-6-(2- | |
| | (dimethylamino)ethoxy)-3,4- | |
| | dihydroisoquinolin-2(1H)-yl)methanone | |
| | Prepared from 2-(benzyloxy)-4- | |
| | (difluoromethyl)-6-hydroxybenzoic acid | |
| | (Intermediate D) and 2-((8-(difluoromethyl)- | |
| | 1,2,3,4-tetrahydroisoquinolin-6-yl)oxy)-N,N- | |
| | dimethylethan-1-amine hydrochloride | |
| | (Intermediate U) | |
| 4.75 | 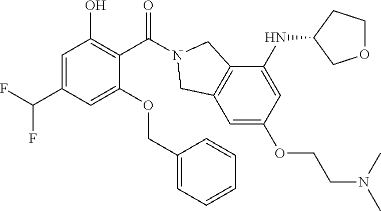 [SMILES: CN(C)CCOc1cc2c(c(N[C@@H]3CCOC3)c1)CN(C(=O)c1c(O)cc(C(F)F)cc1OCc1ccccc1)C2] | ¹H NMR (DMSO-d6, 400 MHz): δ 1.85-1.87 (m, 1H), 2.13-2.20 (m, 6H), 2.50-2.59 (m, 3H), 3.50-4.05 (m, 7H), 4.33 (d, J = 20 Hz, 2H), 4.57 (s, 1H), 4.67 (s, 1H), 5.13 (s, 2H), 5.95-6.24 (m, 2H), 6.75 (s, br, 1H), 6.82-7.09 (m, 2H), 7.25-7.35 (m, 5H), 10.25 (s, 1H). LCMS (Method N): 1.318 min, MS ES+ : 568.1 (M + 1). HPLC (Method I): 6.79 min. |
| | (R)-(2-(Benzyloxy)-4-(difluoromethyl)-6- | |
| | hydroxyphenyl) (6-(2- | |
| | (dimethylamino)ethoxy)-4-((tetrahydrofuran- | |
| | 3-yl)amino)isoindolin-2-yl)methanone | |
| | Prepared from 2-(benzyloxy)-4- | |
| | (difluoromethyl)-6-hydroxybenzoic acid | |
| | (Intermediate D) and (R)-6-(2- | |
| | (dimethylamino)ethoxy)-N-(tetrahydrofuran- | |
| | 3-yl)isoindolin-4-amine hydrochloride | |
| | (Intermediate H) | |
| 4.76 | 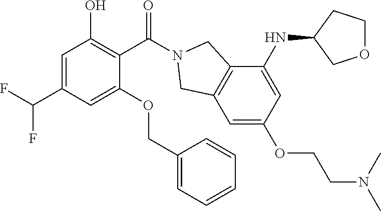 [SMILES: CN(C)CCOc1cc2c(c(N[C@H]3CCOC3)c1)CN(C(=O)c1c(O)cc(C(F)F)cc1OCc1ccccc1)C2] | ¹H NMR (DMSO-d6, 400 MHz): δ 1.85-1.87 (m, 1H), 2.12-2.20 (m, 6H), 2.50-2.60 (m, 3H), 3.48-4.04 (m, 7H), 4.26-4.35 (m, 2H), 4.54 (s, 1H), 4.64 (s, 1H), 5.16 (s, 2H), 5.50 (dd, J = 4 Hz, 1H, D₂O exchangeable), 5.95-6.23 (m, 2H), 6.74-7.08 (m, 3H), 7.24-7.34 (m, 5H), 10.25 (s, 1H). LCMS (Method N): 1.342 min, MS ES+ : 568.1 (M + 1). HPLC (Method F): 4.65 min. |
| | ((S)-(2-(Benzyloxy)-4-(difluoromethyl)-6- | |
| | hydroxyphenyl)(6-(2- | |
| | (dimethylamino)ethoxy)-4-((tetrahydrofuran- | |
| | 3-yl)amino)isoindolin-2-yl)methanone | |
| | Prepared from 2-(benzyloxy)-4- | |
| | (difluoromethyl)-6-hydroxybenzoic acid | |
| | (Intermediate D) and (S)-6-(2- | |
| | (dimethylamino)ethoxy)-N-(tetrahydrofuran- | |
| | 3-yl)isoindolin-4-amine hydrochloride | |
| | (Intermediate I) | |
| 4.77 | 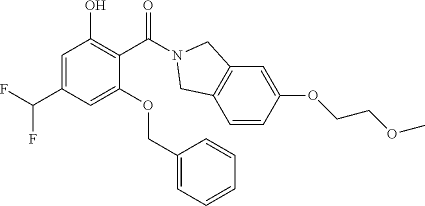 [SMILES: COCCOc1ccc2c(c1)CN(C(=O)c1c(O)cc(C(F)F)cc1OCc1ccccc1)C2] | HT ¹H NMR (DMSO-d6, 400 MHz, 348.2K): δ 3.26 (s, 3H), 3.62-3.67 (m, 2H), 4.02-4.10 (m, 2H), 4.40 (bs, 2H), 4.72 (d, J = 15.2 Hz, 2H), 5.17 (s, 2H), 6.77-6.95 (m, 4H), 6.98-7.13 (m, 2H), 7.25-7.34 (m, 5H), 10.25 (s, 1H). LCMS (Method N): 1.686 min, MS: ES+ 470.1 (M + 1). HPLC (Method F): 7.78 min. |
| | (2-(Benzyloxy)-4-(difluoromethyl)-6- | |
| | hydroxyphenyl)(5-(2- | |
| | methoxyethoxy)isoindolin-2-yl)methanone | |
| | Prepared from 2-(benzyloxy)-4- | |
| | (difluoromethyl)-6-hydroxybenzoic acid | |
| | (Intermediate D) and 5-(2- | |
| | methoxyethoxy)isoindoline hydrochloride | |
| | (Intermediate VD) | |
| 4.78 | 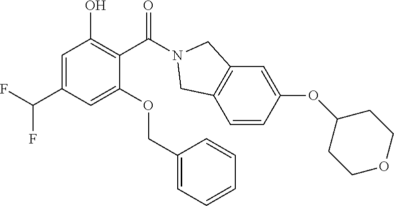 [SMILES: O=C(c1c(O)cc(C(F)F)cc1OCc1ccccc1)N1Cc2ccc(OC3CCOCC3)cc2C1] | ¹H NMR (DMSO-d6, 400 MHz): δ 1.53-1.58 (m, 2H), 1.93-1.95 (m, 2H), 3.46-3.50 (m, 2H), 3.81-3.87 (m, 2H), 4.37-4.56 (m, 3H), 4.72 (d, J = 15.2 Hz, 2H), 5.17 (s, 2H), 6.75 (bs, 1H), 6.82-6.93 (m, 3H), 6.96- 7.16 (m, 2H), 7.13-7.33 (m, 5H), 10.29 (bs, 1H). LCMS (Method N): 1.699 min, MS ES+ : 496.0 (M + 1). HPLC (Method F): 7.953 min. |
| | (2-(Benzyloxy)-4-(difluoromethyl)-6- | |
| | hydroxyphenyl) (5-((tetrahydro-2H-pyran-4- | |
| | yl)oxy)isoindolin-2-yl)methanone | |
| | Prepared from 2-(benzyloxy)-4- | |
| | (difluoromethyl)-6-hydroxybenzoic acid | |
| | (Intermediate D) and 5-((tetrahydro-2H- | |
| | pyran-4-yl)oxy)isoindoline hydrochloride | |
| | (Intermediate VE) | |
| 4.79 | 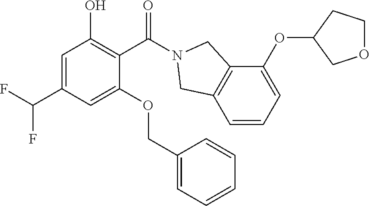 [SMILES: O=C(c1c(O)cc(C(F)F)cc1OCc1ccccc1)N1Cc2cccc(OC3CCOC3)c2C1] | ¹H NMR (DMSO-d6, 400 MHz): δ 1.86-2.03 (m, 1H), 2.16-2.33 (m, 1H), 3.67-3.94 (m, 4H), 4.24-4.38 (m, 1H), 4.45-4.78 (m, 1H), 4.64 (s, 1H), 4.78 (s, 1H), 5.04-5.11 (m, 1H), 5.17-5.31 (m, 2H), 6.75 (s, 1H), 6.85-6.92 (m, 2H), 6.96-7.07 (m, 1H), 7.09-7.10 (m, 1H), 7.25- 7.43 (m, 6H), 10.29 (s, 1H). LCMS (Method W): 6.89 min, MS: ES+ 482.2 (M + 1). HPLC (Method H): 7.66 min. |
| | (2-(Benzyloxy)-4-(difluoromethyl)-6- | |
| | hydroxyphenyl)(4-((tetrahydrofuran-3- | |
| | yl)oxy)isoindolin-2-yl)methanone | |
| | Prepared from 2-(benzyloxy)-4- | |
| | (difluoromethyl)-6-hydroxybenzoic acid | |
| | (Intermediate D) and 4-((tetrahydrofuran-3- | |
| | yl)oxy)isoindoline (Intermediate ZV) | |
| 4.80 | 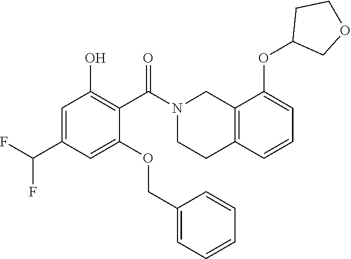 [SMILES: O=C(c1c(O)cc(C(F)F)cc1OCc1ccccc1)N1CCc2cccc(OC3CCOC3)c2C1] | HT ¹H NMR (DMSO-d6, 400 MHz, 348K): δ 1.98 (m, 1H), 2.33 (m, 1H), 2.62-2.67 (m, 2H), 3.42 (m, 1H), 3.69-3.80 (m, 3H), 3.88-3.90 (m, 2H), 4.33-4.35 (m, 1H), 4.76 (m, 1H), 4.97-5.03 (m, 2H), 5.13 (s, 1H), 6.59-6.90 (m, 5H), 7.04-7.28 (m, 6H), 9.88 (s, 1H). LCMS (Method N): 1.73 min, MS: ES+ 496.1 (M + 1). HPLC (Method H): 8.04 min. |
| | 2-(Benzyloxy)-4-(difluoromethyl)-6- | |
| | hydroxyphenyl)(8-((tetrahydrofuran-3- | |
| | yl)oxy)-3,4-dihydroisoquinolin-2(1H)- | |
| | yl)methanone | |
| | Prepared from 2-(benzyloxy)-4- | |
| | (difluoromethyl)-6-hydroxybenzoic acid and | |
| | 8-((tetrahydrofuran-3-yl)oxy)-1,2,3,4- | |
| | tetrahydroisoquinoline (Intermediate ZW) | |

### Example 5: (S)-(2-(Benzyloxy)-4-fluoro-6-hydroxyphenyl)(4-((tetrahydrofuran-3-yl)amino)isoindolin-2-yl)methanone

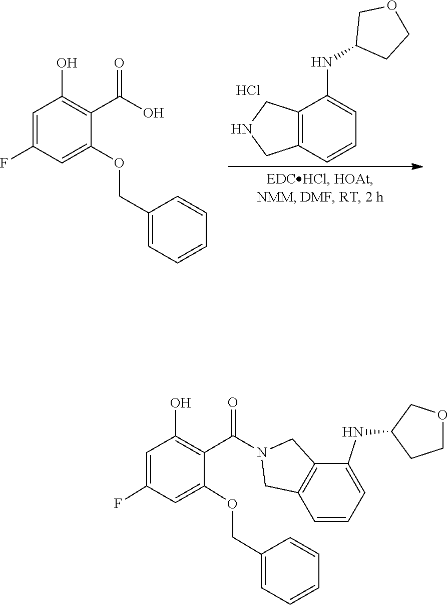 [SMILES: O=C(O)c1c(O)cc(F)cc1OCc1ccccc1 ; Cl ; c1cc2c(c(N[C@H]3CCOC3)c1)CNC2 ; O=C(c1c(O)cc(F)cc1OCc1ccccc1)N1Cc2cccc(N[C@H]3CCOC3)c2C1]

To a stirred solution of 2-(benzyloxy)-4-fluoro-6-hydroxybenzoic acid (Intermediate E) (0.15 g, 0.57 mmol, 1 eq.) in DMF (1.5 mL) were added EDC·HCl (0.164 g, 0.85 mmol, 1.5 eq.) and HOAT (0.077 g, 0.57 mmol, 1 eq.) and the mixture was stirred at room temperature for 15 mins. (S)—N-(Tetrahydrofuran-3-yl)isoindolin-4-amine hydrochloride (Intermediate A) (0.164 g, 0.68 mmol, 1.2 eq.) and NMM (0.3 mL, 2.85 mmol, 5 eq.) were added and stirring continued at room temperature for 2 h. The resulting mixture was diluted with water (20 mL) and extracted with EtOAc (3×15 mL). The combined organic extracts were dried over Na₂SO₄, filtered and concentrated under vacuum. The crude material was purified by chromatography on silica (product eluted at 3.2% MeOH in DCM) to yield the title compound as an off-white solid (0.06 g, Yield: 23.4%).

¹H NMR (DMSO-d6, 400 MHz): δ 1.77-1.86 (m, 1H), 2.12-2.20 (m, 1H), 3.35-3.60 (m, 1H), 3.67-3.76 (m, 2H), 3.81-4.03 (m, 3H), 4.33-4.39 (m, 2H), 4.59 (s, 1H), 4.68 (s, 1H), 5.13 (s, 2H), 5.34-5.47 (m, 1H, D₂O exchangeable), 6.35 (d, J=4 Hz, 1H), 6.43-6.49 (m, 1H), 6.54-6.62 (m, 2H), 7.05-7.13 (m, 1H), 7.26-7.30 (m, 5H), 10.28 (s, 1H).

LCMS (Method A): 1.963 min, MS: ES+ 449.1 (M+1).

HPLC (Method D): 7.97 min.

### Example 6: (R)-(2-(Benzyloxy)-4-fluoro-6-hydroxyphenyl)(4-((tetrahydrofuran-3-yl)amino)isoindolin-2-yl)methanone

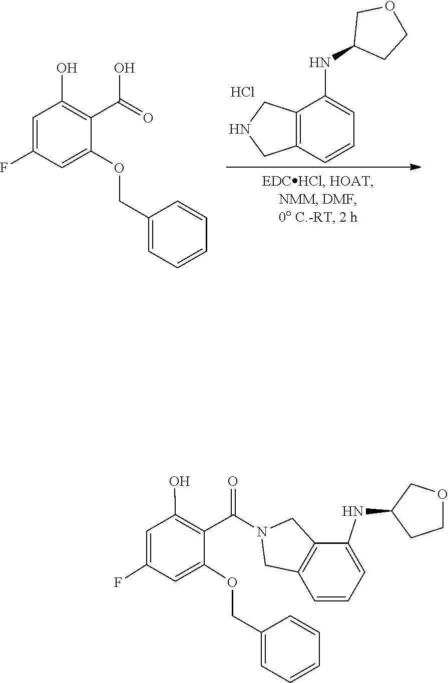 [SMILES: O=C(O)c1c(O)cc(F)cc1OCc1ccccc1 ; Cl ; c1cc2c(c(N[C@@H]3CCOC3)c1)CNC2 ; O=C(c1c(O)cc(F)cc1OCc1ccccc1)N1Cc2cccc(N[C@@H]3CCOC3)c2C1]

To a stirred solution of 2-(benzyloxy)-4-fluoro-6-hydroxybenzoic acid (Intermediate E) (0.1 g, 0.38 mmol, 1 eq.) in DMF (1 mL) were added EDC·HCl (0.1 g, 0.57 mmol, 1.5 eq.) and HOAT (0.052 g, 0.38 mmol, 1 eq.) and the mixture was stirred at room temperature for 15 mins. (R)—N-(Tetrahydrofuran-3-yl)isoindolin-4-amine hydrochloride (Intermediate B) (0.077 g, 0.38 mmol, 1 eq.) and NMM (0.2 mL, 1.90 mmol, 5 eq.) were added and stirring continued at room temperature for 2 h. The resulting mixture diluted with water (20 mL) and extracted with EtOAc (3×20 mL). The combined organic extracts were dried over Na₂SO₄, filtered and concentrated under vacuum. The crude material was purified by chromatography on silica (product eluted at 4% MeOH in DCM) to yield the title compound as a white solid (0.047 g, Yield: 27.5%)

¹H NMR (DMSO-d6, 400 MHz): δ 1.71-1.90 (m, 1H), 2.18-2.11 (m, 1H), 3.52-3.59 (m, 1H), 3.67-3.77 (m, 1H), 3.77-4.04 (m, 3H), 4.39, 4.35 (t, J=16 Hz, 2H), 4.59 (s, 1H), 4.68 (s, 1H), 5.13 (d, J=4 Hz, 2H), 5.47-5.95 (m, 1H, D₂O exchangeable), 6.35-6.31 (m, 1H), 6.49-6.43 (m, 1H), 6.57 (d, J=1.6 Hz, 1H), 6.60 (d, J=4 Hz, 1H), 7.13-7.05 (m, 1H), 7.32-7.23 (m, 5H), 9.97 (s, 1H).

LCMS (Method A): 1.979 min, MS: ES+ 449 (M+1).

HPLC (Method D): 7.96 min.

### Example 7: (R)-(2-(Benzyloxy)-4-chloro-6-hydroxyphenyl)(4-((tetrahydrofuran-3-yl)amino)isoindolin-2-yl)methanone

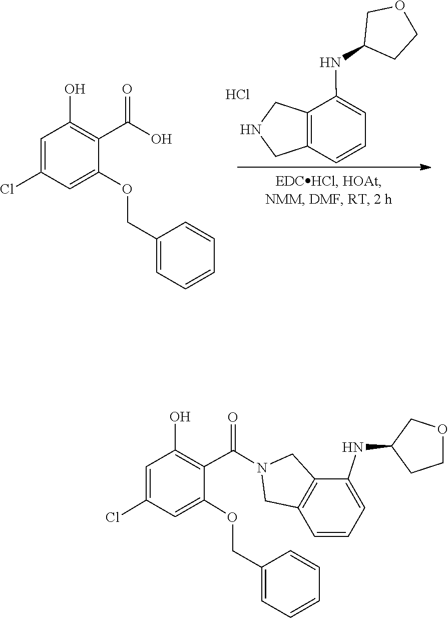 [SMILES: O=C(O)c1c(O)cc(Cl)cc1OCc1ccccc1 ; Cl ; c1cc2c(c(N[C@@H]3CCOC3)c1)CNC2 ; O=C(c1c(O)cc(Cl)cc1OCc1ccccc1)N1Cc2cccc(N[C@@H]3CCOC3)c2C1]

To a stirred solution of 2-(benzyloxy)-4-chloro-6-hydroxybenzoic acid (Intermediate F) (0.15 g, 0.53 mmol, 1 eq.) in DMF (1.5 mL) were added EDC·HCl (0.15 g, 0.80 mmol, 1.5 eq.) and HOAT (0.073 g, 0.53 mmol, 1 eq.) and the mixture was stirred at room temperature for 15 mins. (R)—N-(Tetrahydrofuran-3-yl)isoindolin-4-amine hydrochloride (Intermediate B) (0.11 g, 0.53 mmol, 1.2 eq.) and NMM (0.3 mL, 2.69 mmol, 5 eq.) were added and stirring continued at room temperature for 2 h. The resulting mixture was diluted with water (20 mL) and extracted with EtOAc (3×15 mL). The combined organic extracts were dried over Na₂SO₄, filtered and concentrated under vacuum. The crude material was purified by chromatography on silica (product eluted at 2.7% MeOH in DCM) to yield the title compound as a white solid (0.04 g, Yield: 16.0%)

¹H NMR (DMSO-d6, 400 MHz): δ 1.77-1.86 (m, 1H), 2.12-2.20 (m, 1H), 3.49-3.53 (m, 1H), 3.68-3.77 (m, 1H), 3.81-4.04 (m, 2H), 4.29-4.39 (m, 2H), 4.64-4.68 (m, 2H), 5.16 (s, 2H), 5.35-5.49 (m, 1H, D₂O exchangeable), 6.43-6.49-6.62 (m, 3H), 6.74 (s, 1H), 7.06-7.13 (m, 1H), 7.28-7.30 (m, 4H), 10.30 (s, 1H).

LCMS (Method A): 2.066 min, MS: ES+ 465 (M+1).

HPLC (Method D): 8.323 min.

### Example 8: (S)-(2-(Benzyloxy)-4-chloro-6-hydroxyphenyl)(4-((tetrahydrofuran-3-yl)amino)isoindolin-2-yl)methanone

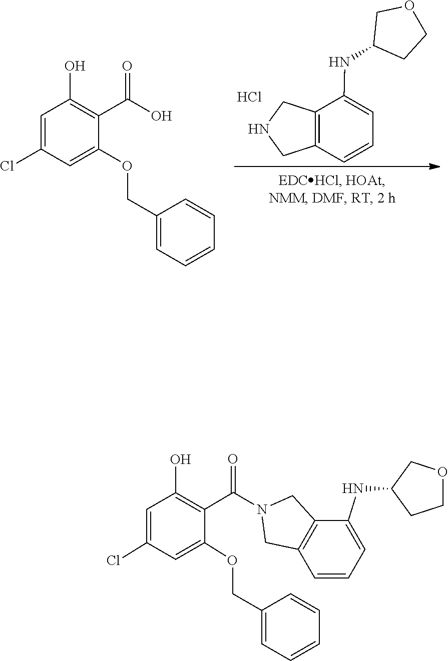 [SMILES: O=C(O)c1c(O)cc(Cl)cc1OCc1ccccc1 ; Cl ; c1cc2c(c(N[C@H]3CCOC3)c1)CNC2 ; O=C(c1c(O)cc(Cl)cc1OCc1ccccc1)N1Cc2cccc(N[C@H]3CCOC3)c2C1]

To a stirred solution of 2-(benzyloxy)-4-chloro-6-hydroxybenzoic acid (Intermediate F) (0.1 g, 0.35 mmol, 1 eq.) in DMF (1 mL) were added EDC·HCl (0.10 g, 0.53 mmol, 1.5 eq.) and HOAT (0.048 g, 0.53 mmol, 1 eq.) and the mixture was stirred at room temperature and for 15 mins. (S)—N-(Tetrahydrofuran-3-yl)isoindolin-4-amine hydrochloride (Intermediate A) (0.073 g, 0.035 mmol, 1.0 eq.) and NMM (0.2 mL, 1.79 mmol, 5 eq.) were added and stirring continued at room temperature for 2 h. The resulting mixture was diluted with water (10 mL) and extracted with EtOAc (3×15 mL). The combined organic extracts were dried over Na₂SO₄, filtered and concentrated under vacuum. The crude material was purified by chromatography on silica (product eluted at 3.7% MeOH in DCM) to yield the title compound as a white solid (0.035 g, Yield: 21.0%)

High temperature 1H NMR (DMSO-d6, 400 MHz): δ 1.78-1.90 (m, 1H), 2.12-2.20 (m, 1H), 3.51-3.54 (m, 1H), 3.62-3.89 (m, 3H), 3.84-4.02 (m, 1H), 4.37-4.42 (m, 2H), 4.63-4.71 (m, 2H), 5.14 (d, J=4 Hz, 2H), 5.34-5.50 (m, 1H, D₂O exchangeable), 6.46-6.50 (m, 1H), 6.61-6.63 (m, 2H), 6.73 (d, J=1.6 Hz, 1H), 7.06-7.13 (m, 1H), 7.24-7.33 (m, 5H), 9.98 (s, 1H).

LCMS (Method A): 2.060 min, MS: ES+ 465 (M+1).

HPLC (Method C): 5.80 min.

### Example 9: (R)-(2-(Benzyloxy)-4-chloro-6-hydroxyphenyl)(6-(2-(dimethylamino)ethoxy)-4-((tetrahydrofuran-3-yl)amino)isoindolin-2-yl)methanone

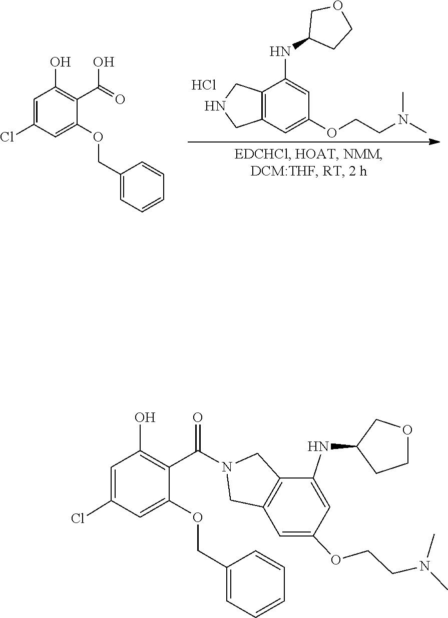 [SMILES: O=C(O)c1c(O)cc(Cl)cc1OCc1ccccc1 ; Cl ; CN(C)CCOc1cc2c(c(N[C@@H]3CCOC3)c1)CNC2 ; CN(C)CCOc1cc2c(c(N[C@@H]3CCOC3)c1)CN(C(=O)c1c(O)cc(Cl)cc1OCc1ccccc1)C2]

To a stirred solution of 2-(benzyloxy)-4-chloro-6-hydroxybenzoic acid (Intermediate F) (0.15 g, 0.53 mmol, 1 eq.) in DMF (4.5 mL) were added EDC·HCl (0.155 g, 0.80 mmol, 1.5 eq.) and HOAT (0.073 g, 0.53 mmol, 1 eq.) and the mixture was stirred at room temperature for 30 mins. (R)-6-(2-(Dimethylamino)ethoxy)-N-(tetrahydrofuran-3-yl)isoindolin-4-amine hydrochloride (Intermediate H) (0.212 g, 0.64 mmol, 1.2 eq.) and NMM (0.272 g, 2.69 mmol, 5 eq.) were added and stirring continued at room temperature for 2 h. The resulting mixture was diluted with water (100 mL) and extracted with EtOAc (3×50 mL). The combined organic extracts were dried over Na₂SO₄, filtered and concentrated under vacuum. The crude material was purified by chromatography on silica (product eluted at 7% MeOH in DCM) to yield the title compound as a white solid (0.050 g, Yield: 17.0%).

¹H NMR (DMSO-d6, 400 MHz): δ 1.75-1.89 (m, 1H), 2.08-2.20 (m, 1H), 2.29-2.32 (m, 6H), 2.67-2.75 (m, 2H), 3.49-3.60 (m, 1H), 3.66-3.75 (m, 2H), 3.82-3.92 (m, 1H), 3.96-4.05 (m, 3H), 4.25-4.34 (m, 2H), 4.52-4.63 (m, 2H), 5.15 (s, 2H), 5.40-5.54 (m, 1H, D₂O exchangeable), 5.98 (d, J=13.2 Hz, 1H), 6.10-6.23 (m, 1H), 6.59-6.74 (m, 2H), 7.24-7.32 (m, 5H), 10.30 (s, 1H).

LCMS (Method A): 1.480 min, MS: ES+ 551.9 & 553.7 (M+1 & M+3).

HPLC (Method D): 5.422 min.

### Example 10: (S)-(2-(Benzyloxy)-4-chloro-6-hydroxyphenyl)(6-(2-(dimethylamino)ethoxy)-4-((tetrahydrofuran-3-yl)amino)isoindolin-2-yl)methanone

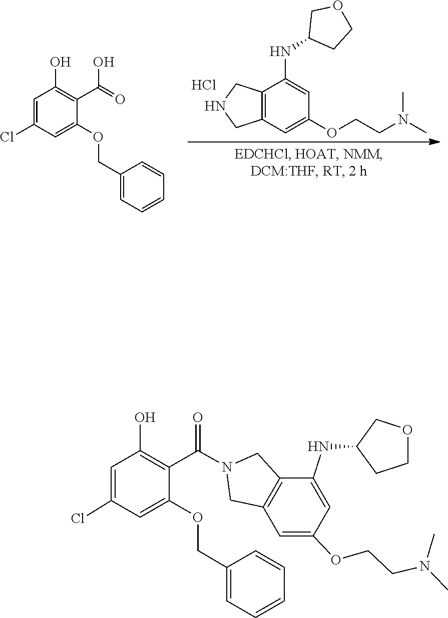 [SMILES: O=C(O)c1c(O)cc(Cl)cc1OCc1ccccc1 ; Cl ; CN(C)CCOc1cc2c(c(N[C@H]3CCOC3)c1)CNC2 ; CN(C)CCOc1cc2c(c(N[C@H]3CCOC3)c1)CN(C(=O)c1c(O)cc(Cl)cc1OCc1ccccc1)C2]

To a stirred solution of 2-(benzyloxy)-4-chloro-6-hydroxybenzoic acid (Intermediate F) (0.15 g, 0.53 mmol, 1 eq.) in DMF (4.5 mL) were added EDC·HCl (0.155 g, 0.80 mmol, 1.5 eq.) and HOAT (0.073 g, 0.53 mmol, 1 eq.) and the mixture was stirred at room temperature for 30 mins. (S)-6-(2-(Dimethylamino)ethoxy)-N-(tetrahydrofuran-3-yl)isoindolin-4-amine hydrochloride (Intermediate I) (0.212 g, 0.64 mmol, 1.2 eq.) and NMM (0.272 g, 2.69 mmol, 5 eq.) were added and stirring continued at room temperature for 2 h. The resulting mixture was diluted with water (100 mL) and extracted with EtOAc (3×50 mL). The combined organic extracts were dried over Na₂SO₄, filtered and concentrated under vacuum. The crude material was purified by chromatography on silica (product eluted at 7.5% MeOH in DCM) to yield the title compound as a white solid (0.080 g, Yield: 27.0%).

¹H NMR (DMSO-d6, 400 MHz): δ 1.72-1.90 (m, 1H), 2.06-2.20 (m, 1H), 2.34-2.37 (m, 6H), 2.78-2.82 (m, 2H), 3.49-3.60 (m, 1H), 3.66-3.75 (m, 1H), 3.82-3.92 (m, 2H), 3.96-4.07 (m, 3H), 4.25-4.34 (m, 2H), 4.52-4.63 (m, 2H), 5.15 (s, 2H), 5.41-5.55 (m, 1H, D₂O exchangeable), 5.98 (d, J=12.8 Hz, 1H), 6.10-6.24 (m, 1H), 6.60-6.73 (m, 2H), 7.24-7.32 (m, 5H), 10.32 (s, 1H).

LCMS (Method M): 8.349 min, MS: ES+ 552.8 & 553.7 (M+1 & M+3).

The compounds of the following tabulated Examples (Table Ex10) were prepared analogously to Example 10 from the indicated intermediates.

TABLE EX 10

| Ex. | Structure and Name /Ref | Analytical Data |
|---|---|---|
| 10.1 | 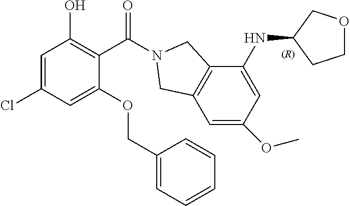 | ¹H NMR (DMSO-d6, 400 MHz): δ 1.75-1.85 (m, 1H), 2.08-2.19 (m, 1H), 3.49-3.60 (m, 1H), 3.65-3.76 (m, 4H), 3.80-4.01 (m, 2H), 4.25- 4.34 (m, 2H), 4.52 (s, 1H), 4.64 (s, 1H), 5.15 (s, 2H), 5.40-5.53 (m, 1H, D₂O exchangeable), 5.97 (d, J = 12.4 Hz, 1H), 6.09-6.22 (m, 1H), 6.58 (s, 1H), 6.73 (s, 1H), 7.24-7.30 (m, 5H), 10.29 (s, 1H). LCMS (Method A): 2.023 min, MS: ES+ 495 & 496.7 (M + 1 & M + 3). HPLC (Method D): 8.178 min. |
| | (R)-(2-(Benzyloxy)-4-chloro-6- | |
| | hydroxyphenyl)(6-methoxy-4- | |
| | ((tetrahydrofuran-3-yl)amino)isoindolin-2- | |
| | yl)methanone | |
| | Prepared from 2-(benzyloxy)-4-chloro-6- | |
| | hydroxybenzoic acid (Intermediate F) and | |
| | (R)-6-methoxy-N-(tetrahydrofuran-3- | |
| | yl)isoindolin-4-amine hydrochloride | |
| | (Intermediate G). | |
| 10.2 | 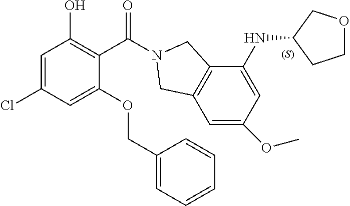 | ¹H NMR (DMSO-d6, 400 MHz): δ 1.71-1.89 (m, 1H), 2.08-2.24 (m, 1H), 3.47-3.52 (m, 1H), 3.55-3.80 (m, 5H), 3.82-4.07 (m, 3H), 4.25- 4.34 (m, 2H), 4.52 (s, 1H), 4.63 (s, 1H), 5.15-5.53 (d, J = 2 Hz, 1H, D₂O exchangeable), 5.97 (dd, J = 2 Hz, 12.0 Hz, 1H), 6.09-6.22 (m, 1H), 6.58 (t, J = 1.6 Hz, 1H), 6.73 (s, 1H), 7.24-7.32 (m, 5H), 10.30 (s, 1H). LCMS (Method A): 2.023 min, MS: ES+ 495 & 496.8 (M + 1 & M + 3). HPLC (Method D): 8.169 min. |
| | (S)-(2-(Benzyloxy)-4-chloro-6- | |
| | hydroxyphenyl)(6-methoxy-4- | |
| | ((tetrahydrofuran-3-yl)amino)isoindolin-2- | |
| | yl)methanone | |
| | Prepared from 2-(benzyloxy)-4-chloro-6- | |
| | hydroxybenzoic acid (Intermediate F) and | |
| | (S)-6-methoxy-N-(tetrahydrofuran-3- | |
| | yl)isoindolin-4-amine hydrochloride | |
| | (Intermediate GA) | |
| 10.3 | 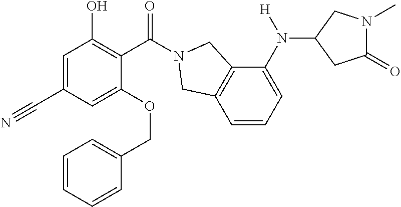 [SMILES: CN1CC(Nc2cccc3c2CN(C(=O)c2c(O)cc(C#N)cc2OCc2ccccc2)C3)CC1=O] | ¹H NMR (DMSO-d6, 400 MHz): δ 2.55-2.76 (m, 4H), 3.62-3.77 (m, 3H), 4.12-4.19 (m, 1H), 4.32-4.40 (m, 2H), 4.64 (s, 1H), 4.73 (s, 1H), 5.20 (d, J = 3.2 Hz, 2H), 5.30-5.50 (m, 1H), 6.45-6.51 (m, 2H), 6.66 (d, J = 7.6 Hz, 1H), 6.92 (d, J = 4.4 Hz, 1H), 7.07-7.15 (m, 2H), 7.26-7.37 (m, 4H). LCMS (Method A): 7.947 min, MS: ES+ 483.5 (M + 1). |
| | 3-(Benzyloxy)-5-hydroxy-4-(4-((1-methyl- | |
| | 5-oxopyrrolidin-3-yl)amino)isoindoline-2- | |
| | carbonyl)benzonitrile | |
| | Prepared from 2-(benzyloxy)-4-cyano-6- | |
| | hydroxybenzoic acid (Intermediate K) and | |
| | 4-(isoindolin-4-ylamino)-1-methylpyrrolidin- | |
| | 2-one hydrochloride (Intermediate L) | |
| 10.4 | 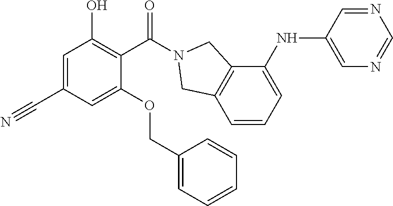 [SMILES: N#Cc1cc(O)c(C(=O)N2Cc3cccc(Nc4cncnc4)c3C2)c(OCc2ccccc2)c1] | ¹H NMR (DMSO-d6, 400 MHz): δ 4.38-4.49 (m, 2H), 4.63-4.81 (m, 2H), 5.32 (bs, 2H), 6.89-7.02 (m, 2H), 7.20-7.30 (m, 8H), 7.95-8.22 (m, 1H, D₂O exchangeable), 8.47- 8.53 (m, 2H), 8.65-8.68 (m, 1H), 10.71 (bs, 1H). LCMS (Method J): 7.999 min. MS: ES+ 464.44 (M + 1). |
| | 3-(Benzyloxy)-5-hydroxy-4-(4-(pyrimidin-5- | |
| | ylamino)isoindoline-2-carbonyl)benzonitrile | |
| | Prepared from 2-(benzyloxy)-4-cyano-6- | |
| | hydroxybenzoic acid (Intermediate K) and | |
| | N-(Pyrimidin-5-yl)isoindolin-4-amine | |
| | hydrochloride (Intermediate OA) | |
| 10.5 | 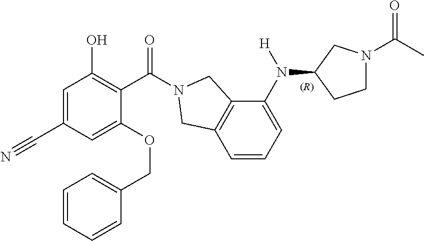 | High Temperature ¹H NMR (DMSO- d6, 400 MHz): δ 1.89-1.97 (m, 3H), 2.05-2.44 (m, 2H), 3.20-3.85 (m, 5H), 4.07-4.17 (m, 1H), 4.35 (bs, 1H), 4.41 (s, 1H), 4.65 (s, 1H), 4.71 (s, 1H), 5.2 (d, J = 2.4 Hz, 2H), 6.49-6.66 (m, 2H), 6.93 (d, J = 5.2, 1H), 7.08- 7.16 (m, 2H), 7.27-7.34 (m, 5H), 10.3 (bs, 1H). LCMS (Method L): 8.499 min, MS: ES+ 497.3 (M + 1). HPLC (Method E): 6.223 min, |
| | (R)-4-(4-((1-Acetylpyrrolidin-3- | |
| | yl)amino)isoindoline-2-carbonyl)-3- | |
| | (benzyloxy)-5-hydroxybenzonitrile | |
| | Prepared from 2-(benzyloxy)-4-cyano-6- | |
| | hydroxybenzoic acid (Intermediate K) and | |
| | (R)-1-(3-(Isoindolin-4-ylamino)pyrrolidin-1- | |
| | yl)ethan-1-one hydrochloride (Intermediate Q) | |
| 10.6 | 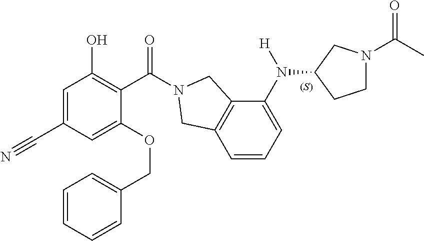 | ¹H NMR (DMSO-d6, 400 MHz): δ 1.87-1.99 (m, 3H), 2.19-2.07 (m, 1H), 3.17-3.18 (m, 1H), 3.40-3.50 (m, 1H), 3.51-3.58 (m, 1H), 3.79- 3.81 (m, 1H), 4.02-4.15 (m, 1H), 4.29-4.39 (m, 2H), 4.61-4.71 (m, 2H), 5.20 (s, 1H), 5.36 (dd, J = 4 Hz, 1H), 5.54 (dd, J = 8 Hz, 1H), 6.47- 6.63 (m, 1H), 6.63 (d, J = 4 Hz, 1H), 6.92 (d, J = 4 Hz, 1H), 7.07-7.15 (m, 1H), 7.18 (s, 1H), 7.52-7.57 (m, 5H), 10.67 (s, 1H). LCMS (Method J): 8.106 min, MS: ES+ 497.57 (M + 1). |
| | (S)-4-(4-((1-Acetylpyrrolidin-3- | |
| | yl)amino)isoindoline-2-carbonyl)-3- | |
| | (benzyloxy)-5-hydroxybenzonitrile | |
| | Prepared from 2-(benzyloxy)-4-cyano-6- | |
| | hydroxybenzoic acid (Intermediate K) and | |
| | (S)-1-(3-(isoindolin-4-ylamino)pyrrolidin-1- | |
| | yl)ethan-1-one (Intermediate QA) | |
| 10.7 | 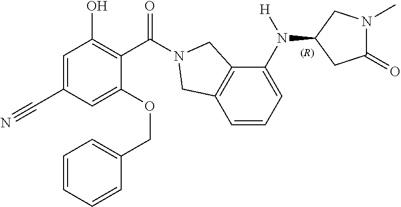 | ¹H NMR (DMSO-d6, 400 MHz): δ 2.18-2.39 (m, 2H), 2.66-2.76 (m, 3H), 3.64-3.77 (m, 2H), 4.12-4.18 (m, 1H), 4.32-4.40 (m, 2H), 4.64- 4.73 (m, 2H), 5.20 (d, J = 3.2 Hz, 2H), 5.32-5.52 (m, 1H, D₂O exchangeable), 6.45-6.71 (m, 2H), 6.93 (d, J = 4.0 Hz, 1H), 7.07-7.14 (m, 2H), 7.26-7.31 (m, 5H), 10.43 (s, 1H). LCMS (Method L): 8.365 min, MS: ES+ 483.4 (M + 1). |
| | or | |
| | 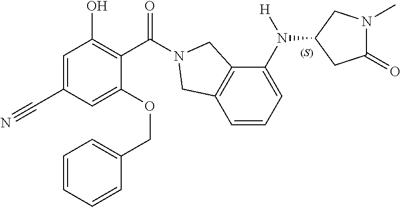 | |
| | (R)-3-(Benzyloxy)-5-hydroxy-4-(4-((1- | |
| | methyl-5-oxopyrrolidin-3- | |
| | yl)amino)isoindoline-2- | |
| | carbonyl)benzonitrile or (S)-3-(benzyloxy)- | |
| | 5-hydroxy-4-(4-((1-methyl-5-oxopyrrolidin- | |
| | 3-yl)amino)isoindoline-2- | |
| | carbonyl)benzonitrile | |
| | Prepared from 2-(benzyloxy)-4-cyano-6- | |
| | hydroxybenzoic acid (Intermediate K) and | |
| | (S)-4-(Isoindolin-4-ylamino)-1- | |
| | methylpyrrolidin-2-one or (R)-4-(isoindolin- | |
| | 4-ylamino)-1-methylpyrrolidin-2-one | |
| | (Intermediate LA). | |
| 10.8 | 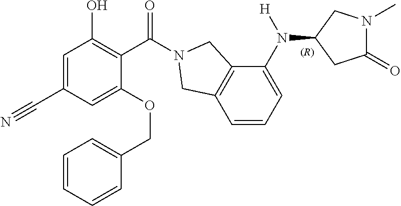 | ¹H NMR (DMSO-d6, 400 MHz): δ 2.19-2.31 (m, 2H), 2.70-2.76 (m, 3H), 3.60-3.75 (m, 2H), 4.13-4.18 (m, 1H), 4.34-4.40 (m, 2H), 4.64- 4.79 (m, 2H), 5.20 (bs, 2H), 5.29- 5.51 (m, 1H, D₂O exchangeable), 6.50 (bs, 1H), 6.65-6.67 (m, 1H), 6.93 (bs, 1H), 7.09-7.13 (m, 2H), 7.27-7.31 (m, 5H), 10.38 (s, 1H). LCMS (Method L): 8.371 min, MS: ES+ 483.3 (M + 1). |
| | or | |
| | 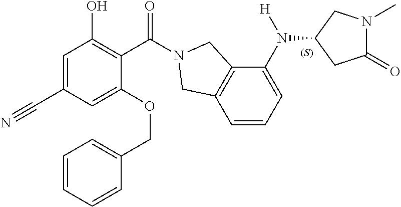 | |
| | (R)-3-(Benzyloxy)-5-hydroxy-4-(4-((1- | |
| | methyl-5-oxopyrrolidin-3- | |
| | yl)amino)isoindoline-2- | |
| | carbonyl)benzonitrile or (S)-3-(benzyloxy)- | |
| | 5-hydroxy-4-(4-((1-methyl-5-oxopyrrolidin- | |
| | 3-yl) amino)isoindoline-2- | |
| | carbonyl)benzonitrile | |
| | Prepared from 2-(benzyloxy)-4-cyano-6- | |
| | hydroxybenzoic acid (Intermediate K) and | |
| | (S)-4-(Isoindolin-4-ylamino)-1- | |
| | methylpyrrolidin-2-one or (R)-4-(isoindolin- | |
| | 4-ylamino)-1-methylpyrrolidin-2-one | |
| | (Intermediate LB). | |
| 10.9 | 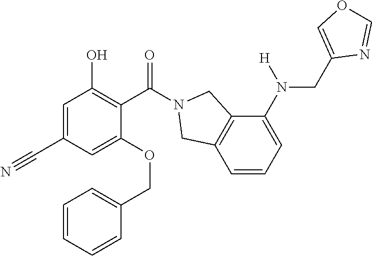 [SMILES: N#Cc1cc(O)c(C(=O)N2Cc3cccc(NCc4cocn4)c3C2)c(OCc2ccccc2)c1] | ¹H NMR (DMSO-d6, 400 MHz): δ 4.13 (d, J = 5.6 Hz, 1H), 4.24 (d, J = 5.6 Hz, 1H), 4.32 (d, J = 7.2 Hz, 1H), 4.38 (d, J = 12 Hz, 1H) 4.63-4.70 (m, 2H), 5.21 (s, 2H), 5.80-5.97 (m, 1H, D₂O exchangeable), 6.45-6.60 (m, 2H), 6.92 (d, J = 7.2 Hz, 2H), 7.02- 7.09 (m, 1H), 7.17-7.18 (m, 1H), 7.24-7.31 (m, 5H), 7.87-7.94 (m, 1H), 8.28-8.33 (m, 1H), 10.67 (s, br, 1H) LCMS (Method J): 8.183 min, MS: ES+ 467.53 (M + 1). HPLC (Method B): 7.47 min. |
| | 3-(Benzyloxy)-5-hydroxy-4-(4-((oxazol-4- | |
| | ylmethyl)amino)isoindoline-2- | |
| | carbonyl)benzonitrile | |
| | Prepared from 2-(benzyloxy)-4-cyano-6- | |
| | hydroxybenzoic acid (Intermediate K) and | |
| | N-(oxazol-4-ylmethyl)isoindolin-4-amine | |
| | hydrochloride (Intermediate IA) | |
| 10.10 | 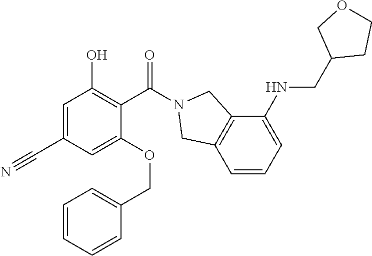 [SMILES: N#Cc1cc(O)c(C(=O)N2Cc3cccc(NCC4CCOC4)c3C2)c(OCc2ccccc2)c1] | ¹H NMR (DMSO-d6, 400 MHz): δ 1.50-1.65 (m, 2H), 1.91-2.01 (m, 2H), 2.95-3.07 (m, 2H), 3.35-3.77 (m, 3H), 4.32-4.39 (m, 2H), 4.63- 4.72 (m, 2H), 5.30 (s, 2H), 5.43-5.55 (m, 1H, D₂O exchangeable), 6.43- 6.58 (m, 2H), 6.93 (d, J = 7.2 Hz, 1H), 7.05-7.14 (m, 1H), 7.17-7.19 (m, 1H), 7.26-7.31 (m, 5H), 10.43 (s, 1H). LCMS (Method J): 8.67 min, MS ES+ : 470.5 (M + 1). |
| | 3-(Benzyloxy)-5-hydroxy-4-(4- | |
| | (((tetrahydrofuran-3- | |
| | yl)methyl)amino)isoindoline-2- | |
| | carbonyl)benzonitrile | |
| | Prepared from 2-(benzyloxy)-4-cyano-6- | |
| | hydroxybenzoic acid (Intermediate K) and | |
| | N-((tetrahydrofuran-3-yl)methyl)isoindolin- | |
| | 4-amine (Intermediate OB) | |
| 10.11 | 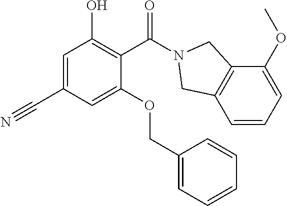 [SMILES: COc1cccc2c1CN(C(=O)c1c(O)cc(C#N)cc1OCc1ccccc1)C2] | ¹H NMR (DMSO-d6, 400 MHz): Mixture of rotamers: δ 3.73 and 3.83 (2 singlets, 3H), 4.21-4.45 (m, 2H), 4.65-4.78 (m, 2H), 5.17-5.24 (m, 2H), 6.82-6.98 (m, 3H), 7.19 (s, 1H), 7.25-7.34 (m, 6H), 10.69 (s, 1H). LCMS (Method A): 2.00 min, MS: ES+ 400.9 (M + 1). HPLC (Method G): 8.493 min. |
| | 3-(Benzyloxy)-5-hydroxy-4-(4- | |
| | methoxyisoindoline-2-carbonyl)benzonitrile | |
| | Prepared from 2-(benzyloxy)-4-cyano-6- | |
| | hydroxybenzoic acid (Intermediate K) and | |
| | 4-methoxyisoindoline hydrochloride (CAS: | |
| | 1203682-51-2) | |
| 10.12 | 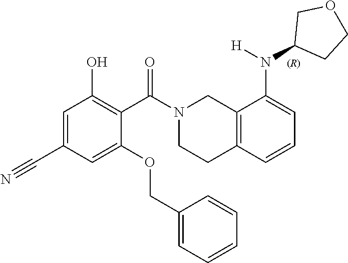 | ¹H NMR (DMSO-d6, 400 MHz): Mixture of rotamers; δ 1.91 (bs 1H), 2.20-2.50 (m, 1H), 2.51-2.75 (m, 1H), 3.37 (t, J = 7.6 Hz, 2H), 3.59- 4.03 (m, 4H), 4.48-4.69 (m, 2H), 4.99- 5.01 (m, 1H), 5.18 (s, 2H), 6.41- 6.52 (m, 2H), 6.89 (s, br, 1H), 6.97- 7.09 (m, 2H), 7.15 (s, 1H), 7.21-7.32 (m, 4H), 10.59 (s, br, 1H). LCMS (Method A): 2.050 min, MS: ES+ 469.9 (M + 1). HPLC (Method B): 8.06 min. |
| | (R)-3-(Benzyloxy)-5-hydroxy-4-(8- | |
| | ((tetrahydrofuran-3-yl)amino)-1,2,3,4- | |
| | tetrahydroisoquinoline-2- | |
| | carbonyl)benzonitrile | |
| | Prepared from 2-(benzyloxy)-4-cyano-6- | |
| | hydroxybenzoic acid (Intermediate K) and | |
| | (R)-N-(tetrahydrofuran-3-yl)-1,2,3,4- | |
| | tetrahydroisoquinolin-8-amine | |
| | hydrochloride (Intermediate OC) | |
| 10.13 | 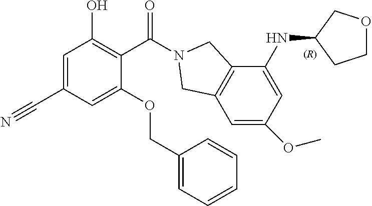 | ¹H NMR (DMSO-d6, 400 MHz): δ 1.71-1.89 (m, 1H), 2.10-2.16 (m, 1H), 3.48-3.61 (m, 1H), 3.65-3.76 (m, 5H), 3.82-4.05 (m, 2H), 4.22- 4.34 (m, 2H), 4.54-4.65 (m, 2H), 5.20 (s, 2H), 5.35-5.53 (m, 1H, D₂O exchangeable), 5.96-6.22 (m, 2H), 6.91 (d, J = 2.4 Hz, 1H), 7.17 (s, 1H), 7.25-7.31 (m, 5H), 10.65 (bs, 1H). LCMS (Method J): 8.051 min, MS: ES+ 486.5 (M + 1). HPLC (Method G): 8.051 min. |
| | (R)-3-(Benzyloxy)-5-hydroxy-4-(6- | |
| | methoxy-4-((tetrahydrofuran-3- | |
| | yl) amino)isoindoline-2- | |
| | carbonyl)benzonitrile | |
| | Prepared from 2-(benzyloxy)-4-cyano-6- | |
| | hydroxybenzoic acid (Intermediate K) and | |
| | (R)-6-methoxy-N-(tetrahydrofuran-3- | |
| | yl)isoindolin-4-amine hydrochloride | |
| | (Intermediate G) | |
| 10.14 | 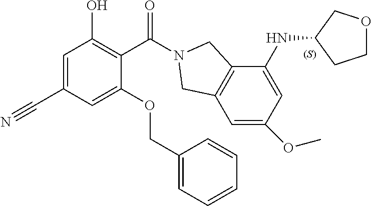 | ¹H NMR (DMSO-d6, 400 MHz): δ 1.75-1.90 (m, 1H), 2.12-2.12 (m, 1H), 3.50-3.61 (m, 1H), 3.65-3.80 (m, 5H), 3.84-3.88 (m, 1H), 3.97- 4.06 (m, 1H), 4.23-4.34 (m, 2H), 4.57 (s, 1H), 4.67 (s, 1H), 5.20 (s, 2H), 5.36-5.54 (s, 1H, D₂O exchangeable), 6.00-6.23 (m, 2H), 6.92 (d, J = 3.6 Hz, 1H), 7.13 (s, 1H), 7.24-7.34 (m, 5H), 10.65 (s, 1H). LCMS (Method J): 8.024 min, MS: ES+ 486.5 (M + 1). HPLC (Method G): 8.024 min. |
| | (S)-3-(Benzyloxy)-5-hydroxy-4-(6- | |
| | methoxy-4-((tetrahydrofuran-3- | |
| | yl) amino)isoindoline-2- | |
| | carbonyl)benzonitrile | |
| | Prepared from 2-(benzyloxy)-4-cyano-6- | |
| | hydroxybenzoic acid (Intermediate K) and | |
| | (S)-6-methoxy-N-(tetrahydrofuran-3- | |
| | yl)isoindolin-4-amine hydrochloride | |
| | (Intermediate GA) | |
| 10.15 | 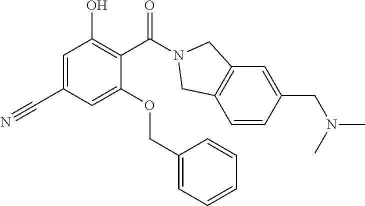 [SMILES: CN(C)Cc1ccc2c(c1)CN(C(=O)c1c(O)cc(C#N)cc1OCc1ccccc1)C2] | ¹H NMR (DMSO-d6, 400 MHz): δ 2.20 (d, J = 4 Hz, 6H), 3.36-3.51 (m, 2H), 4.41-4.50 (m, 2H), 4.77 (s, 2H), 5.20 (s, 2H), 6.94 (s, 1H), 7.19-7.36 (m, 9H), 10.82 (bs, 1H). LCMS (Method K): 5.445 min. MS: ES+ 428.2 (M + 1). HPLC (Method B): 4.76 min. |
| | 3-(Benzyloxy)-4-(5- | |
| | ((dimethylamino)methyl)isoindoline-2- | |
| | carbonyl)-5-hydroxybenzonitrile | |
| | Prepared from 2-(benzyloxy)-4-cyano-6- | |
| | hydroxybenzoic acid (Intermediate K) and | |
| | 1-(Isoindolin-5-yl)-N,N- | |
| | dimethylmethanamine hydrochloride | |
| | (Intermediate R) | |
| 10.16 | 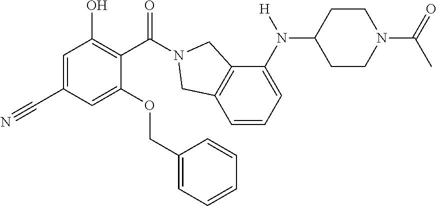 [SMILES: CC(=O)N1CCC(Nc2cccc3c2CN(C(=O)c2c(O)cc(C#N)cc2OCc2ccccc2)C3)CC1] | High temperature ¹H NMR (DMSO- d6, 400 MHz, 349K): δ 1.27-1.52 (m, 2H), 1.87-2.06 (m, 5H), 2.67- 2.82 (m, 1H), 3.51-3.57 (m, 1H), 3.80 (bs, 1H), 4.33 (bs, 2H), 4.40 (s, 1H), 4.64 (s, 1H), 4.72 (s, 1H), 4.78- 4.80 (m, 2H), 5.20 (bs, 2H), 6.44- 6.60 (m, 2H), 6.93 (d, J = 8 Hz, 1H), 7.07-7.14 (m, 2H), 7.26-7.34 (m, 5H), 10.38 (bs, 1H). LCMS (Method J): 8.066 min, MS: ES+ 511.6 (M + 1). HPLC (Method B): 7.016 min, |
| | 4-(4-((1-Acetylpiperidin-4- | |
| | yl)amino)isoindoline-2-carbonyl)-3- | |
| | (benzyloxy)-5-hydroxybenzonitrile | |
| | Prepared from 2-(benzyloxy)-4-cyano-6- | |
| | hydroxybenzoic acid (Intermediate K) and | |
| | 1-(4-(isoindolin-4-ylamino)piperidin-1- | |
| | yl)ethan-1-one hydrochloride (Intermediate | |
| | QB) | |
| 10.17 | 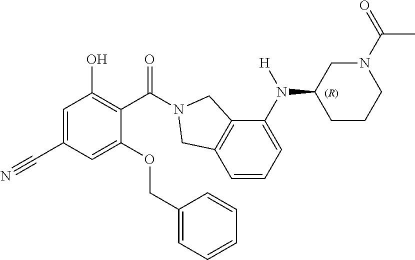 | High temperature ¹H NMR (DMSO- d6, 400 MHz, 349K): δ 1.44-1.45 (m, 1H), 1.56 (m, 1H), 1.60-1.85 (m, 1H), 1.97-2.05 (m, 3H), 2.16 (, Br, 1H), 2.96-3.02 (m, 1H), 3.05-3.30 (m, 1H), 3.74-3.77 (m, 1H), 4.29- 4.39 (m, 2H) 4.50-4.70 (m, 2H), 5.06 (s, br, 1H), 5.20 (d, J = 3.2 Hz, 2H), 6.43-6.62 (m, 2H), 6.93 (d, J = 6 Hz, 1H), 7.08-7.10 (m, 1H), 7.13-7.14 (m, 1H), 7.26-7.45 (m, 5H), 10.36 (s, 1H). ¹H NMR is complex in the aliphatic region due to rotamers. LCMS (Method J): 7.940 min, MS: ES+ 511.54 (M + 1). HPLC (Method G): 7.940 min, |
| | (R)-4-(4-((1-Acetylpiperidin-3- | |
| | yl)amino)isoindoline-2-carbonyl)-3- | |
| | (benzyloxy)-5-hydroxybenzonitrile | |
| | Prepared from 2-(benzyloxy)-4-cyano-6- | |
| | hydroxybenzoic acid (Intermediate K) and | |
| | (R)-1-(3-(isoindolin-4-ylamino)piperidin-1- | |
| | yl)ethan-1-one hydrochloride (Intermediate | |
| | QC) | |
| 10.18 | 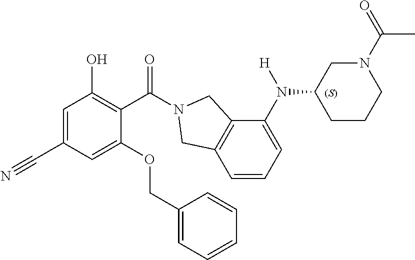 | High temperature ¹H NMR (DMSO- d6, 400 MHz, 349K): δ 1.46- 1.55 (m, 2H), 1.68-1.79 (m, 2H), 1.98 (s, 3H), 2.02 (m, 1H), 3.79 (s, br, 1H), 4.33 (m, 1H), 4.33-4.40 (m, 2H), 4.63 (s, 2H), 4.72-4.79 (m, 1H), 5.20 (s, 2H), 6.46-6.62 (m, 3H), 6.93 (d, J = 6.8 Hz, 1H), 7.08-7.13 (m, 2H), 7.26-7.33 (m, 4H), 10.36 (s, 1H). 1H NMR is complex in the aliphatic region due to rotamers. LCMS (Method J): 8.003 min, MS: ES+ 511.54 (M + 1). |
| | (S)-4-(4-((1-Acetylpiperidin-3- | |
| | yl)amino)isoindoline-2-carbonyl)-3- | |
| | (benzyloxy)-5-hydroxybenzonitrile | |
| | Prepared from 2-(benzyloxy)-4-cyano-6- | |
| | hydroxybenzoic acid (Intermediate K) and | |
| | (S)-1-(3-(Isoindolin-4-ylamino)piperidin-1- | |
| | yl)ethan-1-one hydrochloride (Intermediate QD) | |
| 10.19 | 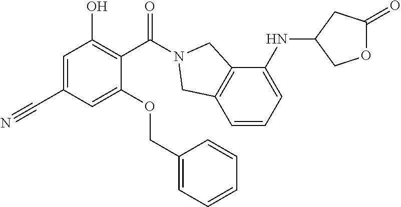 [SMILES: N#Cc1cc(O)c(C(=O)N2Cc3cccc(NC4COC(=O)C4)c3C2)c(OCc2ccccc2)c1] | HPLC (Method G): 8.003 min, ¹H NMR (DMSO-d6, 400 MHz): δ 2.43-2.37 (m, 1H), 3.03-2.91 (m, 1H), 4.17-4.09 (m, 1H), 4.40-4.27 (m, 3H), 4.51-4.47 (m, 1H), 4.72 (s, 2H), 5.21 (d, J = 4 Hz, 2H), 5.87 (dd, J = 4 Hz, 1H), 6.44-6.49 (m, 1H), 6.54 (dd, J = 4 Hz, 1H), 6.93 (d, J = 3.2 Hz, 1H), 7.08-7.18 (m, 2H), 7.30-7.27 (m, 5H), 10.68 (s, 1H). LCMS (Method A): 7.642 min, MS: ES + 470.4 (M + 1). HPLC (Method C): 7.642 min. |
| | 3-(Benzyloxy)-5-hydroxy-4-(4-((5- | |
| | oxotetrahydrofuran-3-yl)amino)isoindoline- | |
| | 2-carbonyl)benzonitrile | |
| | Prepared from 2-(benzyloxy)-4-cyano-6- | |
| | hydroxybenzoic acid (Intermediate K) and | |
| | 4-(isoindolin-4-ylamino)dihydrofuran- | |
| | 2(3H)-one hydrochloride (Intermediate S) | |
| 10.20 | 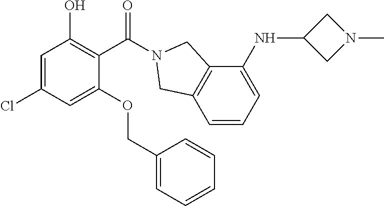 [SMILES: CN1CC(Nc2cccc3c2CN(C(=O)c2c(O)cc(Cl)cc2OCc2ccccc2)C3)C1] | ¹H NMR (DMSO-d6, 400 MHz): δ 2.86 (s, 3H), 3.77-4.03 (m, 2H), 4.25- 4.55 (m, 4H), 4.67 (s, 2H), 5.15 (d, J = 4.4 Hz, 2H), 5.85-6.39 (m, 1H, D₂O exchangeable), 6.34 (s, br 1H), 6.58-6.77 (m, 3H), 7.08-7.17 (m, 1H), 7.26-7.39 (m, 5H), 9.68 (bs, 1H), 10.09 (d, J = 12.8 Hz, 1H). LCMS (Method A): 1.875 min, MS: ES+ 464.2 (M + 1). HPLC (Method D): 5.758 min. |
| | (2-(Benzyloxy)-4-chloro-6- | |
| | hydroxyphenyl)(4-((1-methylazetidin-3- | |
| | yl)amino)isoindolin-2-yl)methanone | |
| | Prepared from 2-(benzyloxy)-4-chloro-6- | |
| | hydroxybenzoic acid (Intermediate F) and | |
| | N-(1-methylazetidin-3-yl) isoindolin-4- | |
| | amine TFA salt (Intermediate NA) | |
| 10.21 | 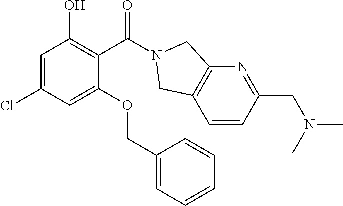 [SMILES: CN(C)Cc1ccc2c(n1)CN(C(=O)c1c(O)cc(Cl)cc1OCc1ccccc1)C2] | ¹H NMR (DMSO-d6, 400 MHz): δ 2.19 (d, J = 15.2 Hz, 6H),3.51-3.47 (m, 2H), 4.39-4.46 (m, 2H), 4.70- 4.78 (m, 2H), 5.16 (s, 2H), 6.60-6.77 (m, 2H), 7.24-7.38 (m, 6H), 7.65- 7.81 (m, 1H), 10.37 (s, 1H). LCMS (Method A): 1.284 min, MS: ES+ 438.0 (M + 1). HPLC (Method D): 5.066 min |
| | 2-(Benzyloxy)-4-chloro-6- | |
| | hydroxyphenyl)(2- | |
| | ((dimethylamino)methyl)-5,7-dihydro-6H- | |
| | pyrrolo[3,4-b]pyridin-6-yl)methanone | |
| | Prepared from 2-(benzyloxy)-4-chloro-6- | |
| | hydroxybenzoic acid (Intermediate F) and | |
| | 1-(6,7-dihydro-5H-pyrrolo[3,4-b]pyridin-2- | |
| | yl)-N,N-dimethylmethanamine | |
| | hydrochloride (Intermediate T) | |
| 10.22 | 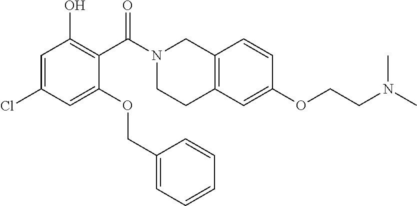 [SMILES: CN(C)CCOc1ccc2c(c1)CCN(C(=O)c1c(O)cc(Cl)cc1OCc1ccccc1)C2] | ¹H NMR (DMSO-d6, 400 MHz): δ 2.28 (s, 6H), 2.67 (t, J = 6 Hz, 2H), 2.80 (s, 2H), 3.45 (s, 1H), 3.65 (s, 1H), 4.07 (t, J = 6 Hz, 2H), 4.34 (s, 1H), 4.72 (s, 1H), 5.01-5.02 (m, 1H), 5.15 (s, 1H), 6.56-6.58 (m, 1H), 6.69- 6.74 (m, 2H), 6.77-6.83 (m, 1H) 6.95-7.17 (m, 2H), 7.21-7.26 (m, 4H), 9.64 (s, 1H). LCMS (Method A): 1.356 min, MS: ES+ 481.1 & 482.8 (M, M + 2). HPLC (Method D): 4.060 min. |
| | (2-(Benzyloxy)-4-chloro-6- | |
| | hydroxyphenyl)(6-(2- | |
| | (dimethylamino)ethoxy)-3,4- | |
| | dihydroisoquinolin-2(1H)-yl)methanone | |
| | Prepared from 2-(benzyloxy)-4-chloro-6- | |
| | hydroxybenzoic acid (Intermediate F) and | |
| | N,N-dimethyl-2-((1,2,3,4- | |
| | tetrahydroisoquinolin-6-yl)oxy)ethan-1- | |
| | amine hydrochloride (Intermediate VF) | |
| 10.23 | 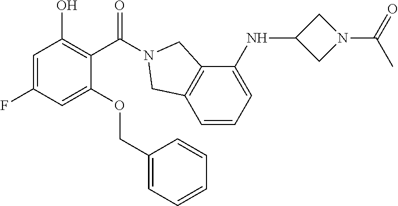 [SMILES: CC(=O)N1CC(Nc2cccc3c2CN(C(=O)c2c(O)cc(F)cc2OCc2ccccc2)C3)C1] | ¹H NMR (DMSO-d6, 400 MHz): δ 1.75 (d, J = 17.6 Hz, 3H), 3.63-3.75 (m, 1H), 3.91-3.94 (m, 1H), 4.09- 4.36 (m, 2H), 4.38-4.44 (m, 3H), 4.62-4.65 (m, 2H), 5.12 (d, J = 4.4 Hz, 2H), 5.84-6.28 (m, 1H, D₂O exchangeable), 6.29-6.36 (m, 2H), 6.53-6.68 (m, 2H), 7.06-7.25 (m, 1H), 7.26-7.31 (m, 5H), 10.29 (s, 1H). LCMS (Method A): 1.725 min, MS: ES+ 476 (M + 1). HPLC (Method C): 5.23 min. |
| | 1-(3-((2-(2-(Benzyloxy)-4-fluoro-6- | |
| | hydroxybenzoyl)isoindolin-4- | |
| | yl)amino)azetidin-1-yl)ethan-1-one | |
| | Prepared from 2-(benzyloxy)-4-fluoro-6- | |
| | hydroxybenzoic acid (Intermediate E) and | |
| | 1-(3-(isoindolin-4-ylamino)azetidin-1- | |
| | yl)ethan-1-one TFA salt (Intermediate N) | |
| 10.24 | 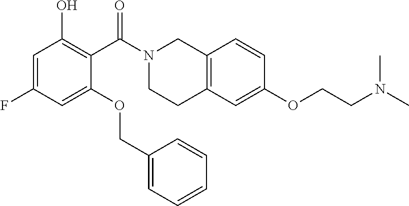 [SMILES: CN(C)CCOc1ccc2c(c1)CCN(C(=O)c1c(O)cc(F)cc1OCc1ccccc1)C2] | ¹H NMR (DMSO-d6, 400 MHz): δ 2.19 (s, 6H), 2.59 (t, J = 5.6 Hz, 2H), 2.75-2.70 (m, 2H), 3.42-3.55 (m, 2H), 4.00 (t, J = 5.6 Hz, 2H), 4.29 (s, 1H), 4.67 (s, 1H), 4.91-5.01 (m, 1H), 5.09 (s, 1H), 6.28-6.31 (m, 1H), 6.50 (t, J = 12.4 Hz, 1H), 6.69-6.80 (m, 3H), 6.91-7.14 (m, 1H), 7.22-7.43 (m, 4H), 10.20 (s, 1H). LCMS (Method A): 1.819 min, MS ES+ : 465 (M + 1). HPLC (Method A): 4.27 min, |
| | (2-(Benzyloxy)-4-fluoro-6- | |
| | hydroxyphenyl)(6-(2- | |
| | (dimethylamino)ethoxy)-3,4- | |
| | dihydroisoquinolin-2(1H)-yl)methanone | |
| | Prepared from 2-(benzyloxy)-4-fluoro-6- | |
| | hydroxybenzoic acid (Intermediate E) and | |
| | N,N-dimethyl-2-((1,2,3,4- | |
| | tetrahydroisoquinolin-6-yl)oxy)ethan-1- | |
| | amine hydrochloride (Intermediate VF) | |
| 10.25 | 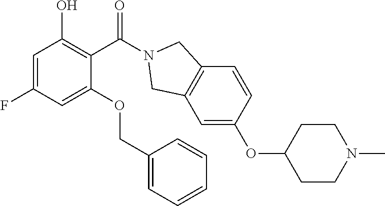 [SMILES: CN1CCC(Oc2ccc3c(c2)CN(C(=O)c2c(O)cc(F)cc2OCc2ccccc2)C3)CC1] | ¹H NMR (DMSO-d6, 400 MHz): δ 1.70-1.66 (m, 2H), 1.91 (s, 1H), 2.67- 2.41 (m, 2H), 4.41-4.26 (m, 3H), 4.68 (d, J = 14.8 Hz, 1H), 5.13 (s, 2H), 6.31-6.34 (m, 1H), 6.54-6.57 (m, 1H), 6.83-6.98(m, 2H), 7.12-7.32 (m, 6H), 10.40 (bs, 1H). LCMS (Method A): 1.857 min, MS: ES+ 477.02 (M + 1). HPLC (Method A): 4.40 min. |
| | (2-(Benzyloxy)-4-fluoro-6-hydroxyphenyl) | |
| | (5-((1-methylpiperidin-4-yl) oxy) isoindolin- | |
| | 2-yl) methadone | |
| | Prepared from 2-(benzyloxy)-4-fluoro-6- | |
| | hydroxybenzoic acid (Intermediate E) and | |
| | 5-((1-methylpiperidin-4-yl)oxy)isoindoline | |
| | hydrochloride (Intermediate V). | |
| 10.26 | 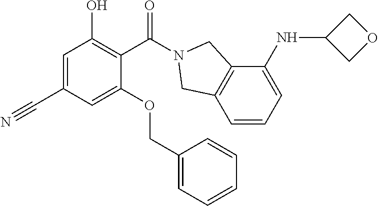 [SMILES: N#Cc1cc(O)c(C(=O)N2Cc3cccc(NC4COC4)c3C2)c(OCc2ccccc2)c1] | ¹H NMR (DMSO-d6, 400 MHz): δ 4.30-4.42 (m, 3H), 4.49-4.59 (m, 2H), 4.65-4.80 (m, 4H), 5.20-5.22 (d, J = 6.0 Hz, 2H), 5.91-6.11 (m, 1H, D₂O exchangeable), 6.16-6.21 (m, 1H), 6.50-6.66 (m, 1H), 6.93 (d, J = 9.2 Hz, 1H), 7.03-7.11 (m, 1H), 7.19 (d, J = 7.6 Hz, 1H), 7.24-7.31 (m, 5H), 10.69 (bs, 1H). LCMS (Method V): 7.019 min, MS: ES+ 442.2 (M + 1). HPLC (Method M): 7.019 min |
| | 3-(Benzyloxy)-5-hydroxy-4-(4-(oxetan-3- | |
| | ylamino)isoindoline-2-carbonyl)benzonitrile | |
| | Prepared from 2-(benzyloxy)-4-cyano-6- | |
| | hydroxybenzoic acid (Intermediate K) and | |
| | N-(oxetan-3-yl)isoindolin-4-amine TFA salt | |
| | (Intermediate NB). | |
| 10.27 | 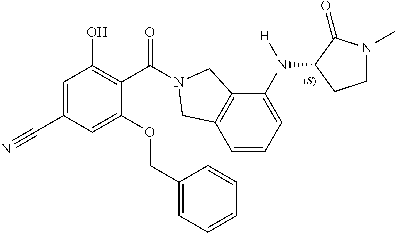 | ¹H NMR (DMSO-d6, 400 MHz): δ 1.74 (bs, 2H), 1.95 (bs, 3H), 2.76 (d, J = 17.6 Hz, 3H), 3.36 (bs, 2H), 4.18- 4.37 (m, 3H), 4.60-4.76 (m, 3H), 5.21 (bs, 2H), 5.38-5.54 (m, 1H), 6.48-6.60 (m, 2H), 6.92 (bs, 1H), 7.07-7.18 (m, 2H), 7.31 (bs, 5H), 10.67 (bs, 1H). LCMS (Method J): 7.715 min, MS: ES+ 483.5 (M + 1). HPLC (Method D): 7.317 min. |
| | (S)-3-(Benzyloxy)-5-hydroxy-4-(4-((1- | |
| | methyl-2-oxopyrrolidin-3- | |
| | yl)amino)isoindoline-2- | |
| | carbonyl)benzonitrile | |
| | Prepared from 2-(benzyloxy)-4-cyano-6- | |
| | hydroxybenzoic acid (Intermediate K) and | |
| | (S)-3-(isoindolin-4-ylamino)-1- | |
| | methylpyrrolidin-2-one hydrochloride | |
| | (Intermediate W). | |
| 10.28 | 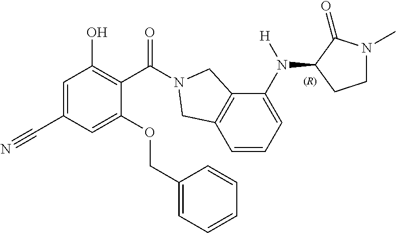 | ¹H NMR (DMSO-d6, 400 MHz): δ 1.69-1.72 (m, 1H), 2.36-2.43 (m, 1H), 2.74 (d, J = 17.6 Hz, 3H), 3.25- 3.30 (m, 1H), 4.14-4.18 (m, 2H), 4.31-4.39 (m, 2H), 4.58-4.61 (m, 1H), 4.71 (bs, 1H), 5.20-5.22 (m, 2H), 5.37-5.54 (m, 1H, D₂O exchangeable), 6.46-6.56 (m, 1H), 6.58-6.61 (m, 1H), 6.91-6.93 (m, 1H), 7.03-7.11 (m, 1H), 7.16 (s, 1H), 7.27-7.31 (m, 5H), 10.19 (bs, 1H). LCMS (Method X): 5.823 min, MS: ES+ 483.3 (M + 1). HPLC (Method D): 6.501 min. |
| | (R)-3-(benzyloxy)-5-hydroxy-4-(4-((1- | |
| | methyl-2-oxopyrrolidin-3- | |
| | yl)amino)isoindoline-2- | |
| | carbonyl)benzonitrile | |
| | Prepared from 2-(benzyloxy)-4-cyano-6- | |
| | hydroxybenzoic acid (Intermediate K) and | |
| | (R)-3-(isoindolin-4-ylamino)-1- | |
| | methylpyrrolidin-2-one hydrochloride | |
| | (Intermediate X) | |
| 10.29 | 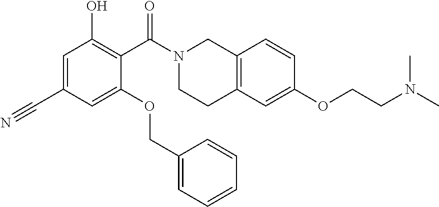 [SMILES: CN(C)CCOc1ccc2c(c1)CCN(C(=O)c1c(O)cc(C#N)cc1OCc1ccccc1)C2] | ¹H NMR (DMSO-d6, 400 MHz): δ 2.20 (s, 6H), 2.65 (t, J = 6.0 Hz, J = 11.6 Hz, 2H), 2.74-2.81 (m, 2H), 3.37-3.42 (m, 2H), 4.06 (t, J = 5.6 Hz, J = 11.6 Hz, 2H), 4.29 (d, J = 8.8 Hz, 1H), 4.71 (s, 1H), 5.07 (d, J = 18.8 Hz, 1H), 5.19 (s, 1H), 6.72-6.94 (m, 4H), 7.08-7.30 (m, 6H), 10.29 (bs, 1H). LCMS (Method N): 1.303 min, MS: ES+ 472.10 (M + 1). HPLC (Method G): 4.33 min. |
| | 3-(Benzyloxy)-4-(6-(2-(dimethyl amino) | |
| | ethoxy)-1,2,3,4-tetrahydroisoquinoline-2- | |
| | carbonyl)-5-hydroxybenzonitrile | |
| | Prepared from 2-(benzyloxy)-4-cyano-6- | |
| | hydroxybenzoic acid (Intermediate K) and | |
| | N, N-dimethyl-2-((1,2,3,4- | |
| | tetrahydroisoquinolin-6-yl) oxy) ethan-1- | |
| | amine (Intermediate VF). | |
| 10.30 | 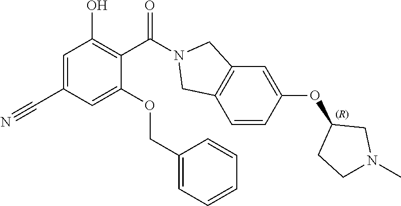 | ¹H NMR (DMSO-d6, 400 MHz): δ 1.74-1.75 (m, 1H), 2.24 (d, J = 9.2 Hz, 3H), 2.28-2.36 (m, 2H), 2.59- 2.67 (m, 2H), 2.71-2.79 (m, 1H), 4.37 (t, J = 14 Hz, 2H), 4.71 (d, J = 14 Hz, 2H), 4.76-4.86 (m, 1H), 5.20 (s, 2H), 6.78-6.91 (m, 3H), 7.12-7.17 (m, 1H), 7.25-7.30 (m, 6H), 10.8 (s, 1H). LCMS (Method V): 7.68 min, MS ES+ : 468.9.(M + 1). HPLC (Method M): 7.68 min, |
| | (R)-3-(Benzyloxy)-5-hydroxy-4-(5-((1- | |
| | methylpyrrolidin-3-yl)oxy)isoindoline-2- | |
| | carbonyl)benzonitrile | |
| | Prepared from 2-(benzyloxy)-4-cyano-6- | |
| | hydroxybenzoic acid (Intermediate K) and | |
| | (R)-5-((1-methylpyrrolidin-3- | |
| | yl)oxy)isoindoline hydrochloride | |
| | (Intermediate VA). | |
| 10.31 | 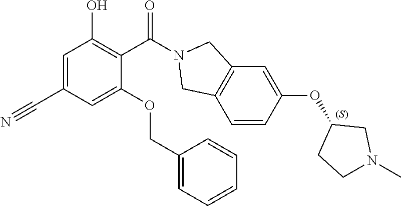 | ¹H NMR (DMSO-d6, 400 MHz): δ 1.70-1.76 (m, 1H), 2.24 (d, J = 9.6, Hz, 3H), 2.28-2.38 (m, 1H), 2.53- 2.67 (m, 3H), 2.71-2.79 (m, 1H), 4.37 (t, J = 16, Hz, 2H), 4.71 (d, J = 14.4, Hz, 1H), 4.76-4.86 (m, 1H), 5.20 (s, 1H), 6.78-6.91 (m, 3H), 7.12-7.37 (m, 7H), 10.76 (bs, 1H). LCMS (Method U): 6.539 min, MS: ES+ 470.3 (M + 1). HPLC (Method I): 6.539 min. |
| | (S)-3-(Benzyloxy)-5-hydroxy-4-(5-((1- | |
| | methylpyrrolidin-3-yl)oxy)isoindoline-2- | |
| | carbonyl)benzonitrile | |
| | Prepared from 2-(benzyloxy)-4-cyano-6- | |
| | hydroxybenzoic acid (Intermediate K) and | |
| | (S)-5-((1-methylpyrrolidin-3- | |
| | yl)oxy)isoindoline hydrochloride | |
| | (Intermediate VB) | |
| 10.32 | 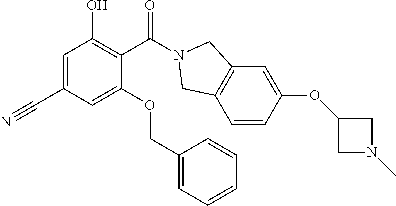 [SMILES: CN1CC(Oc2ccc3c(c2)CN(C(=O)c2c(O)cc(C#N)cc2OCc2ccccc2)C3)C1] | ¹H NMR (DMSO-d6, 400 MHz): δ 2.27 (d, J = 8 Hz, 3H), 2.89-2.96 (m, 2H), 3.68-3.75 (m, 2H), 4.34-4.41 (m, 2H), 4.65-4.74 (m, 3H), 5.20 (s, 2H), 6.72-6.78 (m, 2H), 6.85-6.91 (m, 2H), 7.13-7.17 (m, 1H), 7.26- 7.30 (m, 5H). LCMS (Method U): 1.251 min, 1.222 min, MS ES+ : 456.0.(M + 1). HPLC (Method F): 4.428 min, 4.496 min. |
| | 3-(Benzyloxy)-5-hydroxy-4-(5-((1- | |
| | methylazetidin-3-yl)oxy)isoindoline-2- | |
| | carbonyl)benzonitrile | |
| | Prepared from 2-(benzyloxy)-4-cyano-6- | |
| | hydroxybenzoic acid (Intermediate K) and | |
| | 5-((1-methylazetidin-3-yl)oxy)isoindoline | |
| | TFA salt (Intermediate VC). | |
| 10.33 | 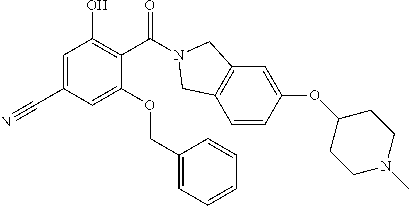 [SMILES: CN1CCC(Oc2ccc3c(c2)CN(C(=O)c2c(O)cc(C#N)cc2OCc2ccccc2)C3)CC1] | ¹H NMR (DMSO-d6, 400 MHz): δ 1.58-1.63 (m, 2H), 1.91 (s, 2H), 2.11- 2.17 (m, 5H), 2.56-2.60 (m, 2H), 4.34-4.41 (m, 3H), 4.71 (d, J = 14 Hz, 2H), 5.20 (s, 2H), 6.84-6.99 (m, 3H), 7.12-7.16 (m, 2H), 7.25-7.31 (m, 5H). LCMS (Method U): 1.267 min, 1.296 min, MS ES+ : 484.1 (M + 1). HPLC (Method L): 4.565 min, 4.633 min. |
| | 3-(Benzyloxy)-5-hydroxy-4-(5-((1- | |
| | methylpiperidin-4-yl)oxy)isoindoline-2- | |
| | carbonyl)benzonitrile | |
| | Prepared from 2-(benzyloxy)-4-cyano-6- | |
| | hydroxybenzoic acid (Intermediate K) and | |
| | 5-((1-methylpiperidin-4-yl)oxy)isoindoline | |
| | TFA salt (Intermediate V). | |
| 10.34 | 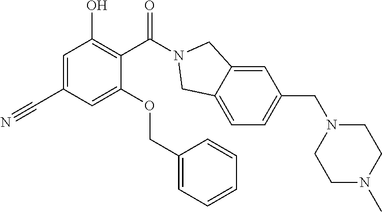 [SMILES: CN1CCN(Cc2ccc3c(c2)CN(C(=O)c2c(O)cc(C#N)cc2OCc2ccccc2)C3)CC1] | ¹H NMR (DMSO-d6, 400 MHz): δ 2.13 (d, J = 7.6 Hz, 4H), 2.18-2.33 (m, 8H), 3.29 (d, J = 12.8 Hz, 2H), 4.42 (d, J = 14.8 Hz, 2H), 4.75 (s, 2H), 5.20 (s, 1H), 6.91 (s, 1H), 7.15-7.19 (m, 3H), 7.22-7.34 (m, 6H), 10.70 (s, 1H) LCMS (Method V): 6.98 min, MS: ES+ 483.4 (M + 1). HPLC (Method M): 6.98 min. |
| | 3-(Benzyloxy)-5-hydroxy-4-(5-((4- | |
| | methylpiperazin-1-yl)methyl)isoindoline-2- | |
| | carbonyl)benzonitrile | |
| | Prepared from 5-((4-Methylpiperazin-1- | |
| | yl)methyl)isoindoline HCl salt (Intermediate | |
| | RA). | |
| 10.35 | 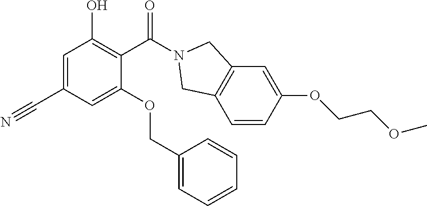 [SMILES: COCCOc1ccc2c(c1)CN(C(=O)c1c(O)cc(C#N)cc1OCc1ccccc1)C2] | HT ¹H NMR (DMSO-d6, 400 MHz, 348K): δ 3.32 (d, J = 9.6 Hz, 3H), 3.63-3.69 (m, 2H), 4.06-4.13 (m, 2H), 4.40 (d, J = 15.2 Hz, 2H), 4.73 (d, J = 15.2 Hz, 2H), 5.20 (s, 2H), 6.84-6.92 (m, 3H), 6.98-7.13 (bs, 2H), 7.26- 7.31 (m, 5H), 10.43 (s, 1H). LCMS (Method N): 1.628 min, MS: ES+ 445 (M + 1) HPLC (Method F): 7.56 min. |
| | 3-(Benzyloxy)-5-hydroxy-4-(5-(2- | |
| | methoxyethoxy)isoindoline-2- | |
| | carbonyl)benzonitrile | |
| | Prepared from 2-(benzyloxy)-4-cyano-6- | |
| | hydroxybenzoic acid (Intermediate K) and | |
| | 5-(2-methoxyethoxy)isoindoline | |
| | hydrochloride (Intermediate VD). | |
| 10.36 | 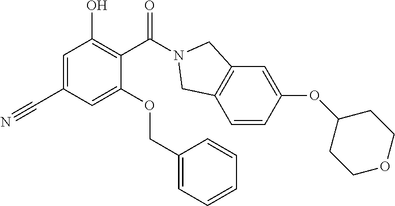 [SMILES: N#Cc1cc(O)c(C(=O)N2Cc3ccc(OC4CCOCC4)cc3C2)c(OCc2ccccc2)c1] | ¹H NMR (DMSO-d6, 400 MHz): δ 1.56 (bs, 2H), 1.95 (bs, 2H), 3.44- 3.51 (m, 2H), 3.84 (bs, 2H), 4.35- 4.42 (m, 3H), 4.72 (d, J = 14 Hz, 2H), 5.21 (s, 2H), 6.89-6.92 (m, 2H), 7.04 (bs, 1H), 7.13-7.18 (m, 2H), 7.27- 7.30 (m, 5H), 10.69 (bs, 1H). LCMS (Method N): 2.220 min, MS ES+ : 470.9 (M + 1), MS ES− 469.0 (M − 1). HPLC (Method F): 7.724 min. |
| | 3-(Benzyloxy)-5-hydroxy-4-(5-((tetrahydro- | |
| | 2H-pyran-4-yl)oxy)isoindoline-2- | |
| | carbonyl)benzonitrile | |
| | Prepared from 2-(benzyloxy)-4-cyano-6- | |
| | hydroxybenzoic acid (Intermediate K) and | |
| | 5-((tetrahydro-2H-pyran-4- | |
| | yl)oxy)isoindoline hydrochloride | |
| | (Intermediate VE). | |
| 10.37 | 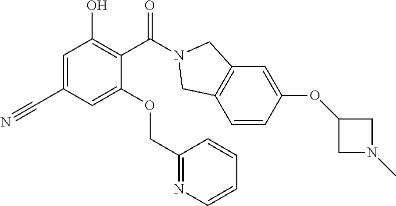 [SMILES: CN1CC(Oc2ccc3c(c2)CN(C(=O)c2c(O)cc(C#N)cc2OCc2ccccn2)C3)C1] | ¹H NMR (DMSO-d6, 400 MHz): δ 2.32 (d, J = 9.6, Hz, 3H), 3.00-3.03 (m, 2H), 3.76-3.80 (m, 2H), 4.45 (t, J = 14 Hz, 2H), 4.67-4.75 (m, 3H), 5.27 (s, 2H), 6.56-6.85 (m, 2H), 6.94 (d, J = 2 Hz, 1H), 7.14-7.36 (m, 4H), 7.73 (t, J = 7.6 Hz, 1H), 8.50 (d, J = 4.4 Hz, 1H), 10.76 (bs, 1H). LCMS (Method V): 5.678 min, MS: ES+ 457.2 (M + 1). HPLC (Method M): 5.678 min. |
| | 3-Hydroxy-4-(5-((1-methylazetidin-3- | |
| | yl)oxy)isoindoline-2-carbonyl)-5-(pyridin-2- | |
| | ylmethoxy)benzonitrile | |
| | Prepared from 4-cyano-2-hydroxy-6- | |
| | (pyridin-2-ylmethoxy)benzoic acid | |
| | (Intermediate KA) and 5-((1- | |
| | methylazetidin-3-yl)oxy)isoindoline | |
| | hydrochloride (hydrochloride salt of | |
| | Intermediate VC). | |
| 10.38 | 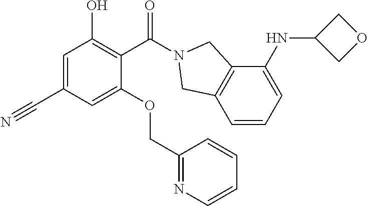 [SMILES: N#Cc1cc(O)c(C(=O)N2Cc3cccc(NC4COC4)c3C2)c(OCc2ccccn2)c1] | ¹H NMR (DMSO-d6, 400 MHz): δ 4.41-4.46 (m, 3H), 4.50-4.61 (m, 2H), 4.70 (m, 1H), 4.75 (m, 1H), 4.79 (t, J = 6.4, Hz, 1H), 4.86 (t, J = 6.4, Hz, 1H), 5.27 (d, J = 4.4 Hz, 1H), 5.71- 6.11 (m, 1H, D₂O exchangeable), 6.16-6.21 (m, 1H), 6.51-6.68 (m, 1H), 6.95 (d, J = 10.4 Hz, 1H), 7.03- 7.17 (m, 2H), 7.27 (t, J = 6.0 Hz, 1H), 7.38 (d, J = 7.6, Hz, 1H), 7.70 (t, J = 7.6 Hz, 1H), 8.50 (t, J = 4.4 Hz, 1H), 10.42 (s, 1H). LCMS (Method V): 5.386 min, MS: |
| | 3-Hydroxy-4-(4-(oxetan-3- | ES+ 443.3 (M + 1). |
| | ylamino)isoindoline-2-carbonyl)-5-(pyridin- | HPLC (Method M): 5.386 min. |
| | 2-ylmethoxy)benzonitrile | |
| | Prepared from 4-cyano-2-hydroxy-6- | |
| | (pyridin-2-ylmethoxy)benzoic acid | |
| | (Intermediate KA) and N-(oxetan-3- | |
| | yl)isoindolin-4-amine TFA salt | |
| | (Intermediate NB). | |
| 10.39 | 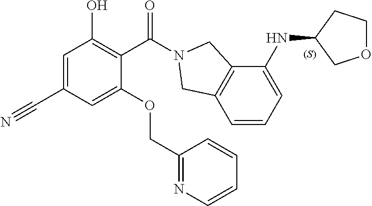 | ¹H NMR (DMSO-d6, 400 MHz): δ 1.76-1.89 (m, 1H), 2.13-2.22 (m, 1H), 3.50-3.62 (m, 1H), 3.68-3.96 (m, 3H), 4.00-4.09 (m, 1H), 4.41 (d, J = 30.8 Hz, 2H), 4.70 (d, J = 31.6 Hz, 2H), 5.08-5.27 (m, 3H), 6.46-6.64 (m, 2H), 6.94 (d, J = 6.8 Hz, 1H), 7.06-7.14 (m, 2H), 7.27 (d, J = 6.0 Hz, 1H), 7.37 (d, J = 7.6 Hz, 1H), 7.68- 7.72 (m, 1H), 8.50 (bs, 1H), 10.35 (bs, 1H). LCMS (Method V): 5.800 min, MS: ES+ 457.3 (M + 1). |
| | (S)-3-Hydroxy-5-(pyridin-2-ylmethoxy)-4- | HPLC (Method M): 5.800 min. |
| | (4-((tetrahydrofuran-3- | |
| | yl)amino)isoindoline-2- | |
| | carbonyl)benzonitrile | |
| | Prepared from 4-cyano-2-hydroxy-6- | |
| | (pyridin-2-ylmethoxy)benzoic acid | |
| | (Intermediate KA) and (S)-N- | |
| | (tetrahydrofuran-3-yl)isoindolin-4-amine | |
| | hydrochloride (Intermediate A). | |

### Example 12: (S)-3-(Benzyloxy)-5-hydroxy-4-(4-((tetrahydrofuran-3-yl)amino)isoindoline-2-carbonyl)benzonitrile as a white solid

### Step 1: 4-(4-Bromoisoindoline-2-carbonyl)-3,5-bis(methoxymethoxy)benzonitrile

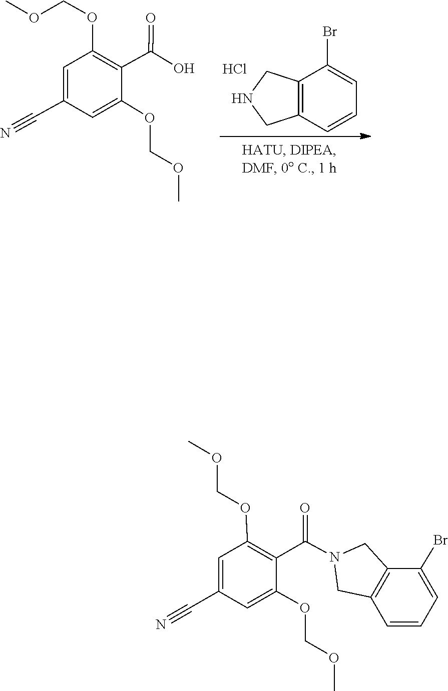 [SMILES: COCOc1cc(C#N)cc(OCOC)c1C(=O)O ; Cl ; Brc1cccc2c1CNC2 ; COCOc1cc(C#N)cc(OCOC)c1C(=O)N1Cc2cccc(Br)c2C1]

To a stirred solution of 4-cyano-2,6-bis(methoxymethoxy)benzoic acid (Intermediate J) (1.5 g, 5.61 mmol, 1 eq.) in DMF (10 mL) were added HATU (3.2 g, 8.4 mmol, 1.5 eq.) and DIPEA (2.2 g, 16.8 mmol, 3 eq.) at 0° C. and the mixture was stirred for 30 mins. 4-Bromoisoindoline hydrochloride (CAS 923590-95-8) (1.6 g, 6.74 mmol, 1.2 eq.) was added and stirring continued at 0° C. for 1 h. The resulting mixture was diluted with ice cold water (200 mL) and extracted with EtOAc (3×50 mL). The combined organic extracts were dried over Na₂SO₄, filtered and concentrated under vacuum. The crude material was purified by chromatography on silica (product eluted at 30% EtOAc in hexane) to yield the title compound as a brown solid (0.750 g, Yield: 30.0%).

¹H NMR (DMSO-d6, 400 MHz): δ 3.30 (s, 6H), 4.43-4.96 (m, 4H), 5.27-5.33 (m, 4H), 7.26-7.55 (m, 5H).

LCMS (Method A): 2.018 min, MS: ES+ 446.8 & 448.8 (M+1 & M+3).

### Step 2: (S)-3,5-bis(Methoxymethoxy)-4-(4-((tetrahydrofuran-3-yl)amino)isoindoline-2-carbonyl)benzonitrile

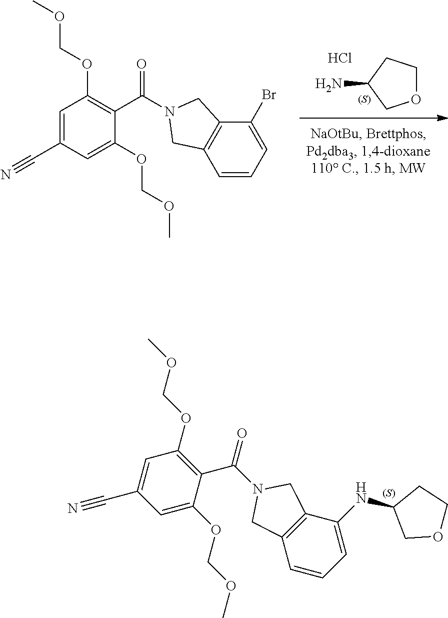

To a stirred solution of 4-(4-bromoisoindoline-2-carbonyl)-3,5-bis(methoxymethoxy)benzonitrile (step 1) (0.75 g, 1.68 mmol, 1 eq.) in 1,4-dioxane (10 mL) were added (S)-tetrahydrofuran-3-amine hydrochloride (CAS 204512-95-8) (0.250 g, 2.0 mmol, 1.2 eq.), NaOtBu (0.403 g, 4.2 mmol, 2.5 eq.) and Brettphos (0.054 g, 0.10 mmol, 0.06 eq.) at room temperature. The mixture was and degassed under N₂ gas for 10 mins. Pd₂(dba)₃ (0.045 g, 0.05 mmol, 0.03 eq.) was added and the resulting mixture was heated to 100° C. using microwave irradiation for 1.5 h. The resulting mixture was diluted with water (50 mL) and extracted with EtOAc (3×30 mL). The combined organic extracts were dried over Na₂SO₄, filtered and concentrated under vacuum. The crude material was purified by chromatography on silica (product eluted at 70% EtOAc in hexane) to yield the title compound as a yellow solid (0.630 g, Yield: 82.0%).

LCMS (Method A): 1.761 min and 1.859, MS: ES+ 454.1 (M+1).

### Step 3: (S)-3,5-Dihydroxy-4-(4-((tetrahydrofuran-3-yl)amino)isoindoline-2-carbonyl)benzonitrile

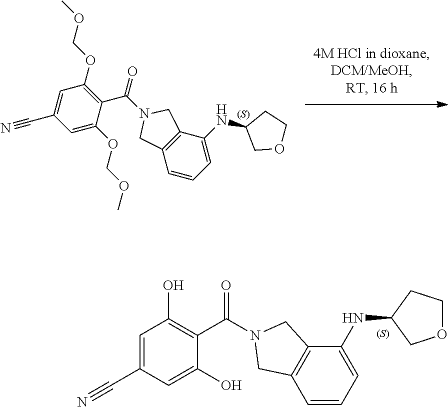

To a stirred solution of (S)-3,5-bis(methoxymethoxy)-4-(4-((tetrahydrofuran-3-yl)amino)isoindoline-2-carbonyl)benzonitrile (step 2) (0.60 g, 1.32 mmol, 1 eq.) in DCM:MeOH (1:1, 1.2 ml) was added dropwise 4M HCl in dioxane (0.6 mL) at 0° C. The reaction mixture was stirred at room temperature for 16 h. The reaction was quenched with saturated solution of NaHCO₃ (30 mL) and the mixture was extracted with 10% MeOH in DCM (7×30 mL). The combined organic extracts were dried over Na₂SO₄, filtered and concentrated under vacuum to yield the title compound as a brown solid (0.5 g, Yield: 82.7%). Further purification

by chromatography on silica (product eluted at 3.5% MeOH in DCM) yielded the title compound as a white solid.

¹H NMR (DMSO-d6, 400 MHz): δ 1.78-1.89 (m, 1H), 2.13-2.21 (m, 1H), 3.50-4.07 (m, 5H), 4.40 (d, J=30.4 Hz, 2H), 4.69 (d, J=30.4 Hz, 2H), 5.12-5.26 (m, 1H), 6.46-6.63 (m, 2H), 6.73 (d, J=4.0 Hz, 2H), 7.06-7.14 (m, 1H), 10.11 (s, 2H).

LCMS (Method A): 1.428 min, MS: ES+ 365.9 (M+1).

HPLC (Method B): 5.97 min.

### Step 4: (S)-3-(Benzyloxy)-5-hydroxy-4-(4-((tetrahydrofuran-3-yl)amino)isoindoline-2-carbonyl)benzonitrile

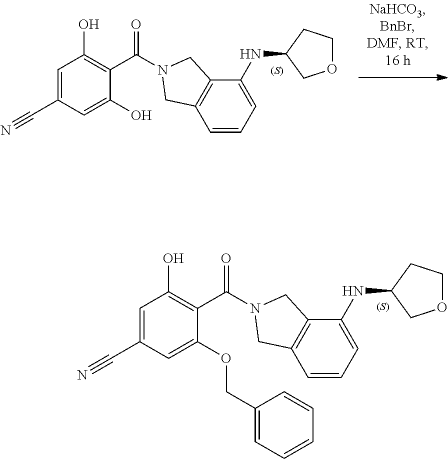

To a stirred solution of (S)-3,5-dihydroxy-4-(4-((tetrahydrofuran-3-yl)amino)isoindoline-2-carbonyl)benzonitrile (step 3) (0.2 g, 0.54 mmol, 1 eq.) in DMF (3 mL) was added NaHCO₃ (0.092 g, 1.09 mmol, 2.0 eq.) and the mixture was stirred at room temperature for 30 mins. The mixture was cooled to 0° C. and treated with benzyl bromide (0.187 g, 1.09 mmol, 2 eq.). The reaction mixture was allowed to warm to room temperature and stirred for 16 h. Purification of the resulting mixture by C18 reverse phase chromatography followed by preparative-HPLC (conditions below) and lyophilization of the pure fractions yielded the title compound as a white solid (0.0055 g, Yield: 1.7%).

¹H NMR (DMSO-d6, 400 MHz): Mixture of rotamers: δ 1.78-1.89 (m, 1H), 2.12-2.20 (m, 1H), 3.51-4.07 (m, 5H), 4.26-4.39 (m, 2H), 4.68 (d, J=29.2 Hz, 1H), 5.21 (s, 3H), 4.30-5.43 (m, 1H, D₂O exchangeable), 6.44-6.62 (m, 3H), 6.93 (d, J=5.2 Hz, 1H), 7.06-7.13 (m, 2H), 7.26-7.32 (m, 4H), 10.35 (bs, 1H).

LCMS (Method J): 8.446 min, MS: ES+ 456.4 (M+1).

HPLC (Method G): 7.83 min

Chromatographic separation and isolation were conducted with a Shimazu NEXERA purification system with UV detector. The column used was SUNFIRE Prep C18 OBD, 19×150 mm, 5 μm and the compounds were eluted with, Mobile Phase A: 0.1% Formic acid in water, Mobile Phase B: Acetonitrile with a gradient of T=0 min (65% A, 35% B); gradient to T=15.00 min (53% A, 47% B); T=15.01 min (45% A, 55% B); gradient to T=18.00 min (45% A, 55% B); T=18.01 min (02% A, 98% B); gradient to T=20.00 min (02% A, 98% B); T=20.01 min (65% A, 35% B) gradient to T=23 min (65% A, 35% B); Flow rate=20 ml/min; analysis time 23 mins.

### Example 12.1: (S)-3-Hydroxy-5-methoxy-4-(4-((tetrahydrofuran-3-yl)amino)isoindoline-2-carbonyl)benzonitrile

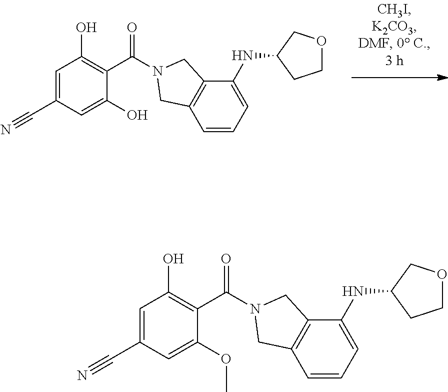 [SMILES: N#Cc1cc(O)c(C(=O)N2Cc3cccc(N[C@H]4CCOC4)c3C2)c(O)c1 ; COc1cc(C#N)cc(O)c1C(=O)N1Cc2cccc(N[C@H]3CCOC3)c2C1]

Performed in 3 parallel batches, each of 1 g scale: To a stirred solution of (S)-3,5-dihydroxy-4-(4-((tetrahydrofuran-3-yl)amino)isoindoline-2-carbonyl)benzonitrile (Example 12 step 3) (0.1 g, 0.27 mmol, 1 eq.) in DMF (4 mL) was added K₂CO₃ (0.038 g, 0.27 mmol, 1 eq.) and the mixture was stirred at room temperature for 30 mins. The mixture was cooled to 0° C., treated dropwise with methyl iodide (0.038 g, 0.27 mmol, 1 eq.) and stirred at 0° C. for 3 h. The resulting mixture was diluted with ice cold water (100 mL) and extracted with EtOAc (6×30 mL). The combined organic extracts were dried over Na₂SO₄, filtered and concentrated under vacuum. The crude material was purified by chromatography on silica (product eluted at 3.8% MeOH in DCM) to yield the title compound as a white solid (0.030 g, Yield: 7.0%).

¹H NMR (DMSO-d6, 400 MHz): δ 1.74-1.93 (m, 1H), 2.15-2.20 (m, 1H), 3.40-3.96 (m, 8H), 4.31-4.39 (m, 2H), 4.64-4.71 (m, 2H), 5.09-5.24 (m, 1H, D₂O exchangeable), 6.46-6.63 (m, 2H), 6.92 (d, J=5.6 Hz, 1H), 7.02-7.14 (m, 2H), 10.35 (s, 1H).

LCMS (Method J): 7.531 min, MS: ES+ 380.3 (M+1).

HPLC (Method G): 7.531 min.

### Example 12.2: (S)-3-(Cyclopropylmethoxy)-5-hydroxy-4-(4-((tetrahydrofuran-3-yl)amino)isoindoline-2-carbonyl)benzonitrile

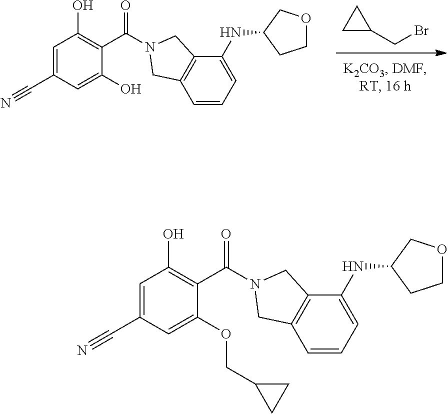

To a stirred solution of (S)-3,5-dihydroxy-4-(4-((tetrahydrofuran-3-yl)amino)isoindoline-2-carbonyl)benzonitrile (Example 12 step 3)) (0.15 g, 0.41 mmol, 1 eq.) in DMF (3 mL) was added K₂CO₃ (0.057 g, 0.41 mmol, 1 eq.) and the mixture was stirred at room temperature for 1 h. Bromomethylcyclopropane (0.070 g, 0.41 mmol, 1 eq.) was dropwise and stirring continued at room temperature for 16 h. The resulting mixture was diluted with ice cold water (50 mL) and extracted with EtOAc (3×30 mL). The combined organic extracts were dried over Na₂SO₄, filtered and concentrated under vacuum. The crude material was purified by chromatography on silica (product eluted at 3.3% MeOH in DCM) to yield the title compound as a white solid (0.024 g, Yield: 7.3%).

¹H NMR (DMSO-d6, 400 MHz): δ 0.21 (d, J=2.8 Hz, 2H), 0.43 (t, J=6.4 Hz, 2H), 1.33-1.35 (m, 1H), 1.74-1.86 (m, 1H), 2.08-2.20 (m, 1H), 3.49-4.03 (m, 7H), 4.29-4.39 (m, 2H), 4.61-4.69 (m, 2H), 5.30-5.49 (m, 1H, D₂O exchangeable), 6.43-6.62 (m, 2H), 6.88 (d, J=4.8 Hz, 1H), 7.06-7.13 (m, 2H), 10.57 (s, 1H).

LCMS (Method J): 8.246 min, MS: ES+ 420.4 (M+1).

HPLC (Method G): 8.246 min.

### Example 12.3: (R)-3-(Benzyloxy)-5-hydroxy-4-(4-((tetrahydrofuran-3-yl)amino)isoindoline-2-carbonyl)benzonitrile

### Step 1: (R)-3,5-Bis(methoxymethoxy)-4-(4-((tetrahydrofuran-3-yl)amino)isoindoline-2-carbonyl)benzonitrile

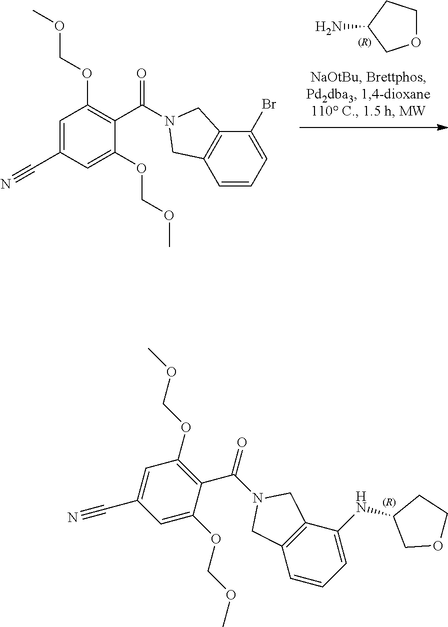

The title compound was prepared from 4-(4-bromoisoindoline-2-carbonyl)-3,5-bis(methoxymethoxy)benzonitrile (Example 12 step 1) and (R)-tetrahydrofuran-3-amine (CAS 111769-26-7) analogously to Example 12 step 2.

LCMS (Method A): 1.791 min, 1.892 min, MS: ES+ 453.9 (M+1).

### Step 2: (R)-3,5-Dihydroxy-4-(4-((tetrahydrofuran-3-yl)amino)isoindoline-2-carbonyl)benzonitrile

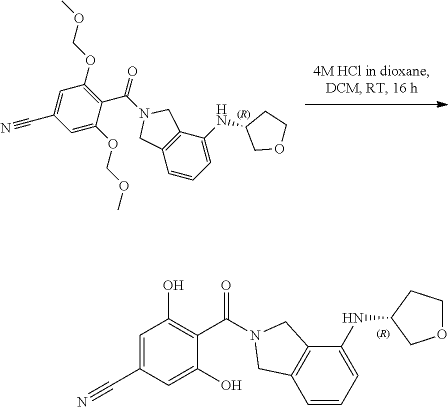

The title compound was prepared from (R)-3,5-bis(methoxymethoxy)-4-(4-((tetrahydrofuran-3-yl)amino)isoindoline-2-carbonyl)benzonitrile (step 1) and 4M HCl in dioxane analogously to Example 12 step 3.

¹H NMR (DMSO-d6, 400 MHz): δ 1.74-1.90 (m, 1H), 2.10-2.18 (m, 1H), 3.48-4.04 (m, 5H), 4.35 (d, J=36.4 Hz, 2H), 4.69 (d, J=36.4 Hz, 2H), 5.33-5.50 (m, 1H), 6.43-6.50 (m, 1H), 6.61 (d, J=7.2 Hz, 1H), 6.72 (d, J=5.2 Hz, 2H), 7.05-7.13 (m, 1H), 10.43 (s, 2H).

LCMS (Method A): 1.425 min, MS: ES+ 365.9 (M+1).

HPLC (Method B): 5.94 min.

### Step 3: (R)-3-(Benzyloxy)-5-hydroxy-4-(4-((tetrahydrofuran-3-yl)amino)isoindoline-2-carbonyl)benzonitrile

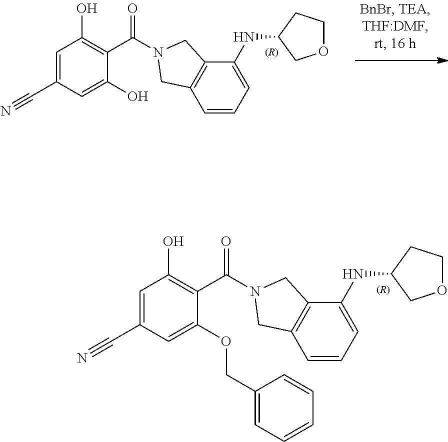

The title compound was prepared from (R)-3,5-dihydroxy-4-(4-((tetrahydrofuran-3-yl)amino)isoindoline-2-carbonyl)benzonitrile (step 2), TEA and benzyl bromide analogously to Example 12 step 4.

¹H NMR (DMSO-d6, 400 MHz): Mixture of rotamers; δ 1.70-1.91 (m, 1H), 2.11-2.18 (m, 1H), 3.48-4.04 (m, 5H), 4.30-4.39 (m, 2H), 4.61-4.70 (m, 2H), 5.21 (d, J=4.4 Hz, 2H), 5.31-5.49 (m, 1H, D₂O exchangeable), 6.43-6.62 (m, 2H), 6.91-6.93 (m, 1H), 7.06-7.18 (m, 2H), 7.24-7.30 (m, 5H), 10.66 (s, 1H).

LCMS (Method J): 8.567 min, MS: ES+ 456.4 (M+1).

HPLC (Method G): 8.567 min.

### Example 12.4: 3-(Benzyloxy)-5-hydroxy-4-(isoindoline-2-carbonyl)benzonitrile

### Step 1: 4-(Isoindoline-2-carbonyl)-3,5-bis(methoxymethoxy)benzonitrile

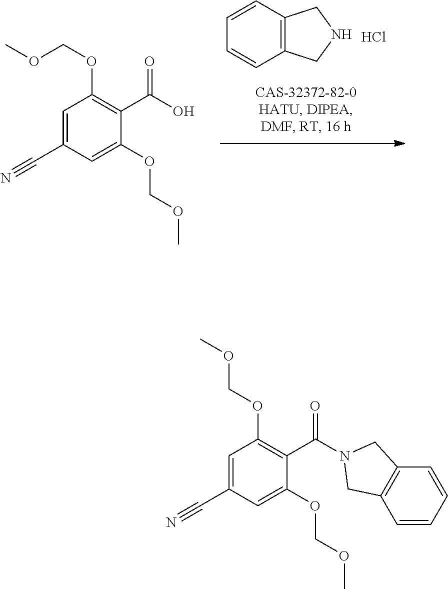 [SMILES: COCOc1cc(C#N)cc(OCOC)c1C(=O)O ; c1ccc2c(c1)CNC2 ; Cl ; COCOc1cc(C#N)cc(OCOC)c1C(=O)N1Cc2ccccc2C1]

The title compound was prepared from 4-cyano-2,6-bis(methoxymethoxy)benzoic acid (Intermediate J) and isoindoline hydrochloride (CAS 32372-82-0) analogously to Example 12 step 1. LCMS (Method A): 1.817 min, MS: ES+ 369.1 (M+1).

### Step 2: 3,5-Dihydroxy-4-(isoindoline-2-carbonyl)benzonitrile

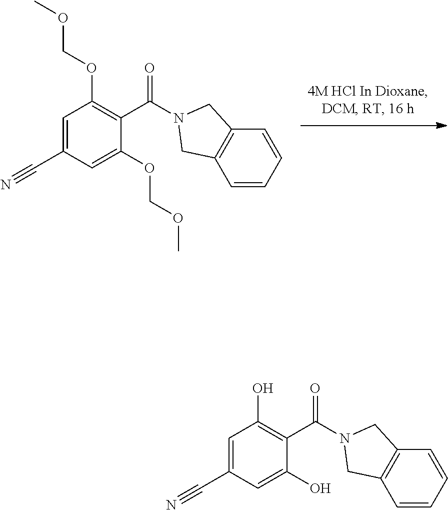 [SMILES: COCOc1cc(C#N)cc(OCOC)c1C(=O)N1Cc2ccccc2C1 ; N#Cc1cc(O)c(C(=O)N2Cc3ccccc3C2)c(O)c1]

The title compound was prepared from 4-(isoindoline-2-carbonyl)-3,5-bis(methoxymethoxy)benzonitrile (step 1) and 4M HCl in dioxane analogously to Example 12 step 3.

¹H NMR (DMSO-d6, 400 MHz): δ 4.49 (s, 2H), 4.77 (s, 2H), 6.73 (s, 2H), 7.28-7.32 (m, 3H), 7.39 (d, J=6.8 Hz, 1H).

LCMS (Method A): 1.399 min, MS: ES+ 280.8 (M+1).

HPLC (Method B): 6.03 min,

### Step 3: 3-(Benzyloxy)-5-hydroxy-4-(isoindoline-2-carbonyl)benzonitrile

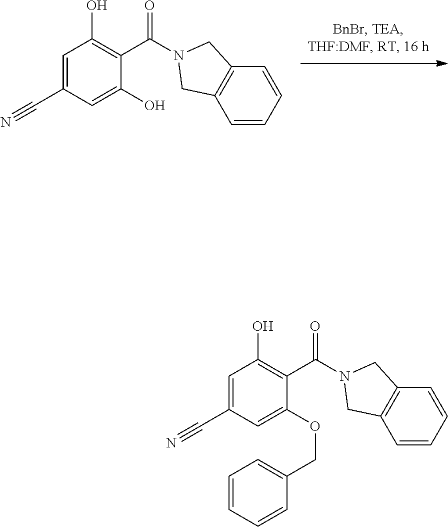 [SMILES: N#Cc1cc(O)c(C(=O)N2Cc3ccccc3C2)c(O)c1 ; N#Cc1cc(O)c(C(=O)N2Cc3ccccc3C2)c(OCc2ccccc2)c1]

The title compound was prepared from 3,5-dihydroxy-4-(isoindoline-2-carbonyl)benzonitrile (step 2), TEA and benzyl bromide analogously to Example 12 step 4.

¹H NMR (DMSO-d6, 400 MHz): δ 4.45 (d, J=14 Hz, 2H), 4.79 (s, 2H), 5.50 (s, 2H), 6.92 (s, 1H), 7.19 (s, 1H), 7.25-7.34 (m, 8H), 7.39 (d, J=7.2 Hz, 1H), 10.69 (s, 1H).

LCMS (Method A): 1.957 min, MS: ES+ 371 (M+1).

HPLC (Method B): 7.93 min.

### Example 13: 3-(Benzyloxy)-5-hydroxy-4-(5-methoxyisoindoline-2-carbonyl)benzonitrile

### Step 1: 4-(5-Methoxyisoindoline-2-carbonyl)-3,5-bis(methoxymethoxy)benzonitrile

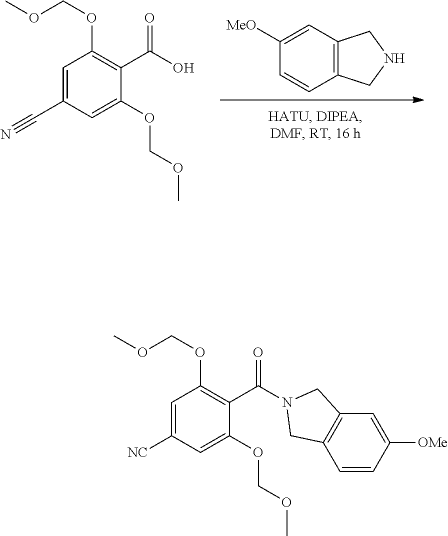 [SMILES: COCOc1cc(C#N)cc(OCOC)c1C(=O)O ; COc1ccc2c(c1)CNC2 ; COCOc1cc(C#N)cc(OCOC)c1C(=O)N1Cc2ccc(OC)cc2C1]

The title compound was prepared from 4-cyano-2,6-bis(methoxymethoxy)benzoic acid (Intermediate J) and 5-methoxyisoindoline (CAS 127168-88-1) analogously to Example 12 Step 1.

¹H NMR (DMSO-d6, 400 MHz): δ 2.94 (s, 3H), 3.30-3.33 (m, 6H), 3.70-3.76 (m, 4H), 4.39-4.43 (m, 2H), 4.66-4.79 (m, 2H), 5.27-5.32 (m, 2H), 6.83-6.90 (m, 2H), 7.38 (d, J=2 Hz, 1H). LCMS (Method A): 1.827 min, MS: ES+ 398.91 (M+1).

### Step 2: 3,5-Dihydroxy-4-(5-methoxyisoindoline-2-carbonyl)benzonitrile

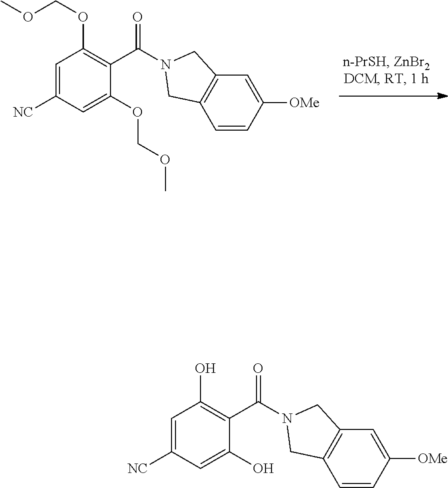 [SMILES: COCOc1cc(C#N)cc(OCOC)c1C(=O)N1Cc2ccc(OC)cc2C1 ; COc1ccc2c(c1)CN(C(=O)c1c(O)cc(C#N)cc1O)C2]

To a stirred solution of 4-(5-methoxyisoindoline-2-carbonyl)-3,5-bis(methoxymethoxy)benzonitrile (step 1) (0.39 g, 0.97 mmol, 1.0 eq.) in DCM (3.9 mL) were added ZnBr (0.99 g, 4.40 mmol, 4.5 eq.) and n-PrSH (0.67 g, 8.81 mmol, 9.0 eq.) and the reaction mixture was stirred at room temperature for 1 h. The resulting mixture was concentrated under vacuum and purification of the crude material by chromatography on silica (product eluted at 10% MeOH in DCM) yielded the title compound. (0.23 g, Yield: 76%).

¹H NMR (DMSO-d6, 400 MHz): δ 3.71-3.75 (m, 3H), 4.40-4.44 (m, 2H), 4.68-4.73 (m, 2H), 6.72 (d, J=1.6 Hz, 2H), 6.83-6.97 (m, 2H), 7.16-7.29 (m, 1H), 10.45 (d, J=3.6 Hz, 2H).

LCMS (Method A): 1.395 min, MS: ES+ 310.74 (M+1).

### Step 3: 3-(Benzyloxy)-5-hydroxy-4-(5-methoxyisoindoline-2-carbonyl)benzonitrile

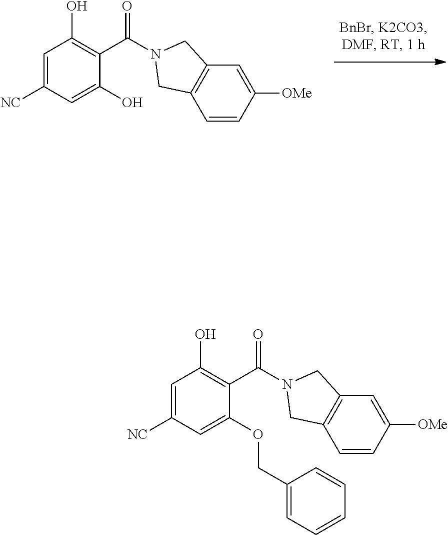 [SMILES: COc1ccc2c(c1)CN(C(=O)c1c(O)cc(C#N)cc1O)C2 ; COc1ccc2c(c1)CN(C(=O)c1c(O)cc(C#N)cc1OCc1ccccc1)C2]

The title compound was prepared from 3,5-dihydroxy-4-(5-methoxyisoindoline-2-carbonyl)benzonitrile (Step 2), NaHCO₃ and benzyl bromide analogously to Example 12 Step 4.

¹H NMR (DMSO-d6, 400 MHz): Mixture of rotamers: δ 3.71 and 3.76 (2 singlets, 3H), 4.34-4.42 (m, 2H), 4.70-4.74 (m, 2H), 5.20 (s, 2H), 6.84-6.98 (m, 3H), 7.14-7.30 (m, 7H), 10.67 (bs, 1H). LCMS (Method A): 1.947 min, MS: ES+ 400.9 (M+1). HPLC (Method B): 7.80 min.

### Example 14: (R)-3-(Benzyloxy)-4-(6-(2-(dimethylamino)ethoxy)-8-((tetrahydrofuran-3-yl)amino)-1,2,3,4-tetrahydroisoquinoline-2-carbonyl)-5-hydroxybenzonitrile

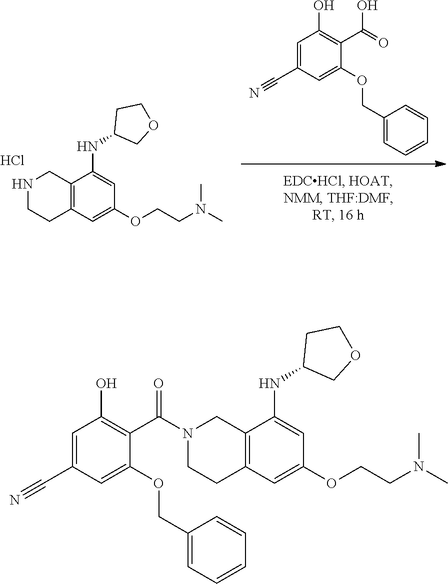 [SMILES: Cl ; CN(C)CCOc1cc2c(c(N[C@@H]3CCOC3)c1)CNCC2 ; N#Cc1cc(O)c(C(=O)O)c(OCc2ccccc2)c1 ; CN(C)CCOc1cc2c(c(N[C@@H]3CCOC3)c1)CN(C(=O)c1c(O)cc(C#N)cc1OCc1ccccc1)CC2]

To a stirred solution 2-(benzyloxy)-4-cyano-6-hydroxybenzoic acid (Intermediate K) (0.15 g, 0.55 mmol, 1 eq.) in THF (0.75 mL) were added EDC·HCl (0.16 g, 0.83 mmol, 1.3 eq.) and HOAT (75 mg, 0.55 mmol, 1 eq.) and the mixture was stirred at room temperature for 5 mins. To this mixture was added (R)-6-(2-(dimethylamino)ethoxy)-N-(tetrahydrofuran-3-yl)-1,2,3,4-tetrahydroisoquinolin-8-amine hydrochloride (Intermediate M) (0.25 g, 0.72 mmol, 1.3 eq.) dissolved in DMF (0.75 mL) and N-methyl morpholine (0.23 g, 1.11 mmol, 2.0 eq.) and stirring continued at room temperature for 16 h. The resulting mixture was poured into ice cold water (100 mL) and extracted with EtOAc (3×50 mL). The combined organic extracts were washed with ice cold water (3×100 mL), dried over Na₂SO₄, filtered and concentrated under vacuum. The crude material was purified by preparative HPLC (conditions below) followed by pure fraction lyophilization to yield the title compound (0.032 g, Yield: 10.3%).

¹H NMR (DMSO-d6, 400 MHz): δ 1.88-1.93 (m, 1H), 2.17-2.20 (m, 1H), 2.21 (s, 6H), 2.59-2.68 (m, 4H), 3.60-3.65 (m, 2H), 3.70-3.76 (m, 1H), 3.82-3.87 (m, 2H), 3.83-3.90 (m, 2H), 3.91-4.01 (m, 5H), 4.33-4.41 (m, 1H), 4.53-4.61 (m, 1H), 5.01-5.05 (m, 1H), 5.18 (s, 2H), 6.01 (s, 2H), 6.88-6.90 (m, 1H), 7.00-7.08 (m, 1H), 7.14 (s, 1H), 7.20-7.23 (m, 1H), 7.26-7.32 (m, 4H), 10.7 (bs, 1H). ¹H NMR is complex in the aliphatic region due to rotamers.

LCMS (Method A): 1.371 min, MS: ES+ 556.99 (M+1).

HPLC (Method B): 4.86 min.

Preparative HPLC: Chromatographic separation and isolation were conducted with a Shimadzu NEXERA purification system with UV detector. The column used was SHIM PACK GIST C18, (250×19) mm, 5 μm and the compounds were eluted with, Mobile Phase A: 0.1% Formic acid in water, Mobile Phase B: Acetonitrile:Methanol:IPA (65:25:10) with a gradient of T=0 min (70% A, 30% B); gradient to T=17.00 min (65% A, 35% B); T=17.01 min (02% A, 98% B); gradient to T=19.00 min (02% A, 98% B); T=19.01 min (70% A, 30% B) gradient to T=21 min (70% A, 30% B); Flow rate=20 ml/min; analysis time 21 mins.

### Example 15: (S)-3-(Benzyloxy)-4-(6-(2-(dimethylamino)ethoxy)-8-((tetrahydrofuran-3-yl)amino)-1,2,3,4-tetrahydroisoquinoline-2-carbonyl)-5-hydroxybenzonitrile

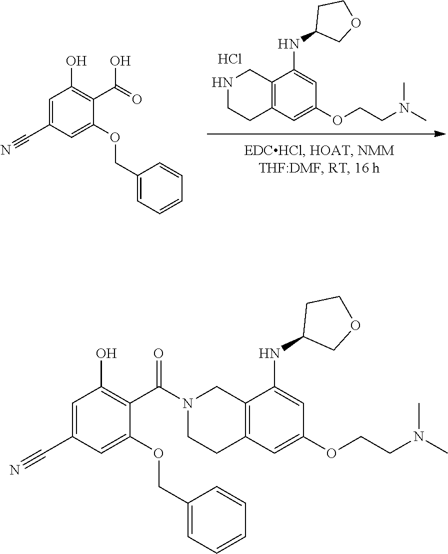 [SMILES: N#Cc1cc(O)c(C(=O)O)c(OCc2ccccc2)c1 ; Cl ; CN(C)CCOc1cc2c(c(N[C@H]3CCOC3)c1)CNCC2 ; CN(C)CCOc1cc2c(c(N[C@H]3CCOC3)c1)CN(C(=O)c1c(O)cc(C#N)cc1OCc1ccccc1)CC2]

To a stirred solution 2-(benzyloxy)-4-cyano-6-hydroxybenzoic acid (Intermediate K) (0.2 g, 0.74 mmol, 1 eq.) in DMF (1 mL) were added EDC·HCl (0.21 g, 1.11 mmol, 1.5 eq.) and HOAT (0.1 g, 0.74 mmol, 1 eq.) and the mixture was stirred at room temperature for 5 mins. To this mixture was added (S)-6-(2-(dimethylamino)ethoxy)-N-(tetrahydrofuran-3-yl)-1,2,3,4-tetrahydroisoquinolin-8-amine hydrochloride (Intermediate MA) (0.33 g, 0.97 mmol, 1.3 eq.) dissolved in DMF (1 mL) and N-methyl morpholine (0.3 g, 0.74 mmol, 2.0 eq.) and the reaction mixture was stirred at room temperature for 16 h. The resulting mixture was poured into ice cold water (80 mL) and extracted with EtOAc (3×40 mL). The combined organic extracts were washed with ice cold water (3×100 mL), dried over Na₂SO₄, filtered and concentrated under vacuum. The crude material was purified by preparative HPLC (conditions below) to yield the title compound (0.033 g, Yield: 11.9%).

¹H NMR (DMSO-d6, 400 MHz): δ 1.89-1.93 (m, 1H), 2.17-2.22 (m, 1H), 2.22 (s, 6H), 2.61-2.68 (m, 4H), 2.62 (s, 2H), 3.37-3.46 (m, 4H), 3.60-3.72 (m, 2H), 3.74-3.82 (m, 2H), 3.83-3.91 (m, 2H), 3.91-3.57 (m, 5H), 4.32-4.41 (m, 1H), 4.53-4.61 (m, 1H), 5.03-5.05 (m, 1H), 5.18 (s, 2H), 6.02 (s, 2H), 6.89-6.90 (m, 1H), 7.00-7.08 (m, 1H), 7.14 (s, 1H), 7.22-7.24 (m, 1H), 7.27-7.33 (m, 4H), 10.7 (bs, 1H). ¹H NMR is complex in the aliphatic region due to rotamers.

Preparative HPLC: Chromatographic separation and isolation were conducted with Waters 2545 purification system with UV detector. The column used was SUNFIRE, C18, OBD 19×250 mm, 5 μm and the compounds were eluted with, Mobile Phase A: 0.05% Formic acid in water, Mobile Phase B: Acetonitrile:Mobile phase-A (80:20) with a gradient of T=0 min (77% A, 23% B); gradient to T=15.00 min (70% A, 30% B); T=15.01 min (02% A, 98% B); gradient to T=17.00 min (02% A, 98% B); T=17.01 min (77% A, 23% B) gradient to T=21 min (77% A, 23% B); Flow rate=17 ml/min; analysis time 21 mins.

### Example 16: 4-(4-((1-Acetylazetidin-3-yl)amino)isoindoline-2-carbonyl)-3-(benzyloxy)-5-hydroxybenzonitrile

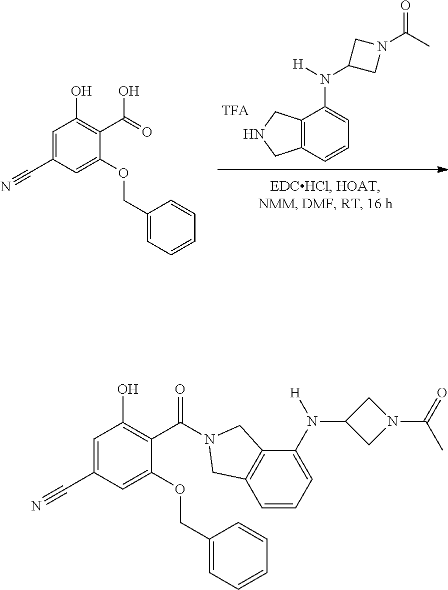 [SMILES: N#Cc1cc(O)c(C(=O)O)c(OCc2ccccc2)c1 ; CC(=O)N1CC(Nc2cccc3c2CNC3)C1 ; "TFA" ; CC(=O)N1CC(Nc2cccc3c2CN(C(=O)c2c(O)cc(C#N)cc2OCc2ccccc2)C3)C1]

To a stirred solution of 2-(benzyloxy)-4-cyano-6-hydroxybenzoic acid (Intermediate K) (0.2 g, 0.74 mmol, 1 eq.) in DCM:THF (5 mL) were added EDC·HCl (0.21 g, 1.11 mmol, 1.5 eq.), HOAT (0.1 g, 0.74 mmol, 1 eq.) followed by 1-(3-(isoindolin-4-ylamino)azetidin-1-yl)ethan-1-one·TFA salt (Intermediate N) (0.23 g, 0.89 mmol, 1.2 eq.) and N-methylmorpholine (0.37 g, 3.71 mmol, 5 eq.) at room temperature. The reaction mixture was stirred at room temperature for 1 h. The resulting mixture was poured into water (50 mL) and extracted with DCM (3×50 mL). The combined organic extracts were dried over Na₂SO₄, filtered and concentrated under vacuum. The crude material was purified using flash chromatography (product eluted at 5% MeOH:DCM) to yield the title compound as a white solid (0.1 g, Yield: 16.7%).

¹H NMR (DMSO-d6, 400 MHz): δ 1.74 (s, 3H), 3.59-3.96 (m, 2H), 4.12-4.28 (m, 2H), 4.38-4.74 (m, 5H), 5.20 (d, J=4.8 Hz, 2H), 5.64, 5.85 (dd, J=6 Hz, 1H), 6.32 (d, J=8 Hz, 1H), 6.53 (d, J=7.6 Hz, 1H), 6.93 (d, J=7.6 Hz, 1H), 7.07-7.15 (m, 2H), 7.25-7.34 (m, 5H), 10.31 (s, 1H).

LCMS (Method J): 7.724 min, MS: ES+ 483.6 (M+1).

HPLC (Method G): 7.724 min,

### Example 17: (S)-3-(Benzyloxy)-5-hydroxy-4-(8-((tetrahydrofuran-3-yl)amino)-1,2,3,4-tetrahydroisoquinoline-2-carbonyl)benzonitrile

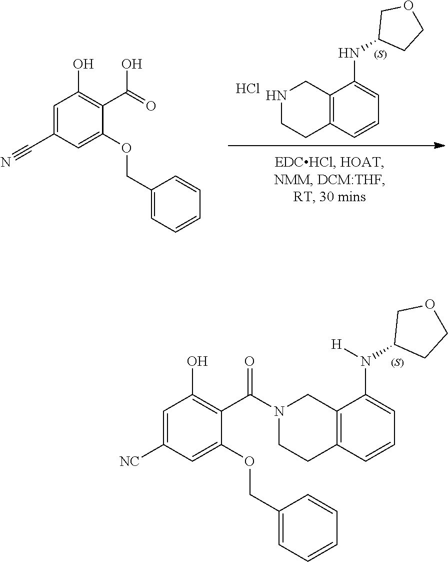

To a stirred solution of 2-(benzyloxy)-4-cyano-6-hydroxybenzoic acid (Intermediate K) (0.2 g, 0.74 mmol, 1 eq.) in DCM:THF (4 mL) were added EDC·HCl (0.21 g, 1.11 mmol, 1.5 eq.), HOAT (0.1 g, 0.74 mmol, 1 eq.) followed by (S)—N-(tetrahydrofuran-3-yl)-1,2,3,4-tetrahydroisoquinolin-8-amine hydrochloride (Intermediate O) (0.245 g, 0.96 mmol, 1.3 eq.) and N-methyl morpholine (0.15 g, 1.48 mmol, 2 eq.) at room temperature. The reaction mixture was stirred at room temperature for 30 mins. The resulting mixture was poured into water (50 mL) and extracted with DCM (3×50 mL). The combined organic extracts were dried over Na₂SO₄ and concentrated under vacuum. The crude material was purified by chromatography on silica (product eluted at 5% MeOH:DCM) to yield the title compound as an off-white solid (0.035 g, Yield: 11.6%).

¹H NMR (DMSO-d6, 400 MHz): Mixture of rotamers; δ 1.91 (bs 1H), 2.20-2.50 (m, 1H), 2.51-2.75 (m, 1H), 3.37-4.02 (m, 6H), 4.40-4.69 (m, 2H), 4.99-5.04 (m, 1H), 5.18 (s, 2H), 6.41-6.52 (m, 2H), 6.87-6.88 (m, 1H), 6.97-7.10 (m, 2H), 7.15 (s, 1H), 7.19-7.32 (m, 4H), 10.59 (s, 1H).

LCMS (Method A): 2.048 min. MS: ES+ 469.9 (M+1).

HPLC (Method B): 8.06 min,

### Example 18: (R)-3-(Benzyloxy)-4-(6-(2-(dimethylamino)ethoxy)-4-((tetrahydrofuran-3-yl)amino)isoindoline-2-carbonyl)-5-hydroxybenzonitrile

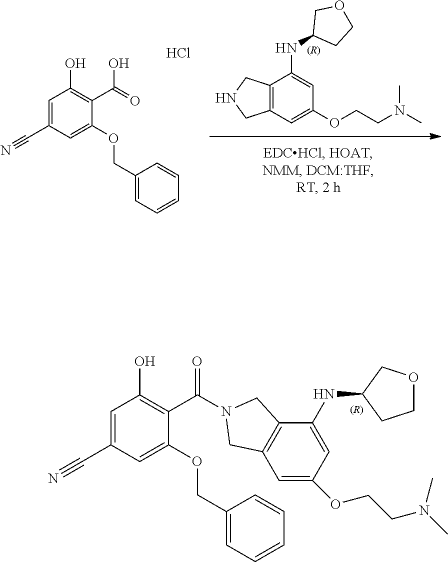

To a stirred solution of 2-(benzyloxy)-4-cyano-6-hydroxybenzoic acid (Intermediate K) (0.15 g, 0.55 mmol, 1 eq.) in DMF (7.5 mL) were added EDC·HCl (0.158 g, 0.82 mmol, 1.5 eq.) and HOAT (0.075 g, 0.55 mmol, 1 eq.) and the mixture was stirred at room temperature for 30 mins. To the mixture were added (R)-6-(2-(dimethylamino)ethoxy)-N-(tetrahydrofuran-3-yl)isoindolin-4-amine hydrochloride (Intermediate H) (0.219 g, 0.66 mmol, 1.2 eq.) and NMM (0.278 mL, 2.75 mmol, 5 eq.) and stirring continued at room temperature for 2 h. The resulting mixture was diluted with water (50 mL) and extracted with EtOAc (5×30 mL). The combined organic extracts were dried over Na₂SO₄, filtered and concentrated under vacuum. The crude material was purified by chromatography on silica (product eluted at 9% MeOH in DCM) to yield the title compound as a white solid (0.050 g, Yield: 17.0%).

¹H NMR (DMSO-d6, 400 MHz): δ 1.76-1.92 (m, 2H), 2.12-2.19 (m, 1H), 2.25 and 2.28 (2 singlets, 6H), 2.68-2.74 (m, 2H), 3.50-3.62 (m, 1H), 3.68-3.95 (m, 3H), 4.00 (t, J=6.0 Hz, 1H), 4.06 (d, J=5.6 Hz, 1H), 4.26 (bs, 1H), 4.34 (s, 1H), 4.57 (s, 1H), 4.67 (s, 1H), 5.20 (s, 2H), 5.35-5.52 (m, 1H, D₂O exchangeable), 5.97-6.24 (m, 2H), 6.93 (d, J=3.2 Hz, 1H), 7.13 (s, 1H), 7.26-7.34 (m, 5H), 10.36 (bs, 1H).

LCMS (Method J): 5.722 min, MS: ES+ 543.6 (M+1).

HPLC (Method G): 5.722 min.

### Example 19: (S)-3-(Benzyloxy)-4-(6-(2-(dimethylamino)ethoxy)-4-((tetrahydrofuran-3-yl)amino)isoindoline-2-carbonyl)-5-hydroxybenzonitrile

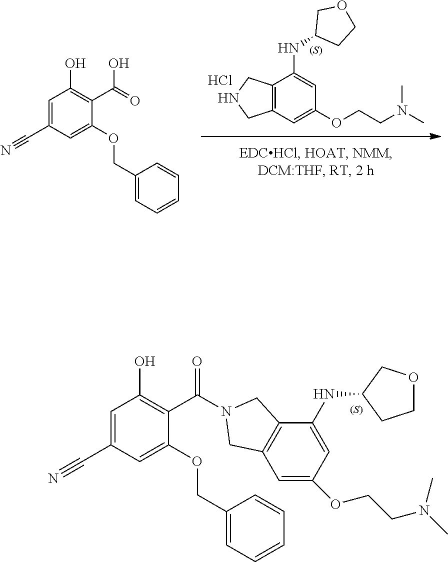

To a stirred solution of 2-(benzyloxy)-4-cyano-6-hydroxybenzoic acid (Intermediate K) (0.15 g, 0.55 mmol, 1 eq.) in DMF (7.5 mL) were added EDC·HCl (0.158 g, 0.82 mmol, 1.5 eq.) and HOAT (0.075 g, 0.55 mmol, 1 eq.) and the mixture was stirred at room temperature for 30 mins. (S)-6-(2-(Dimethylamino)ethoxy)-N-(tetrahydrofuran-3-yl)isoindolin-4-amine hydrochloride (Intermediate I) (0.219 g, 0.66 mmol, 1.2 eq.) and NMM (0.278 mL, 2.75 mmol, 5 eq.) were added and stirring continued at room temperature for 2 h. The resulting mixture was diluted with water (50 mL) and extracted with EtOAc (5×30 mL). The combined organic extracts were dried over Na₂SO₄, filtered and concentrated under vacuum. The crude material was purified by chromatography on silica (product eluted at 8.5% MeOH in DCM) to yield the title compound as a white solid (0.035 g, Yield: 12.0%).

¹H NMR (DMSO-d6, 400 MHz): δ 1.73-1.90 (m, 2H), 2.13-2.20 (m, 1H), 2.24 and 2.28 (2 singlets, 6H), 2.65-2.71 (m, 2H), 3.51-3.64 (m, 1H), 3.67-3.77 (m, 2H), 3.84-4.06 (m, 3H), 4.26 (bs, 1H), 4.34 (s, 1H), 4.57 (s, 1H), 4.67 (s, 1H), 5.20 (s, 3H), 5.36-5.54 (m, 1H, D₂O exchangeable) 5.97-6.23 (m, 2H), 6.93 (dd, J=1.2, 3.6 Hz, 1H), 7.13 (s, 1H), 7.26-7.34 (m, 5H), 10.36 (bs, 1H).

LCMS (Method J): 5.730 min, MS: ES+ 543.6 (M+1).

HPLC (Method G): 5.730 min.

### Example 21: (R)-(2-(Benzyloxy)-4-chloro-6-hydroxyphenyl)(6-(2-(dimethylamino)ethoxy)-8-((tetrahydrofuran-3-yl)amino)-3,4-dihydroisoquinolin-2(1H)-yl)methanone

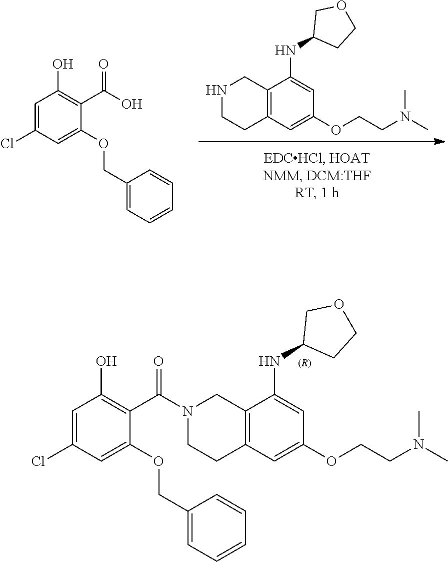

To a stirred solution of 2-(benzyloxy)-4-chloro-6-hydroxybenzoic acid (Intermediate F) (0.3 g, 1.07 mmol, 1 eq.) in DCM:THF (1:1, 8 mL) were added EDC·HCl (0.31 g, 1.61 mmol, 1.5 eq) and HOAT (0.146 g, 1.07 mmol, 1 eq) at room temperature. (R)-6-(2-(Dimethylamino)ethoxy)-N-(tetrahydrofuran-3-yl)-1,2,3,4-tetrahydroisoquinolin-8-amine hydrochloride (Intermediate M) (0.39 g, 1.29 mmol, 1.2 eq) and N-methyl morpholine (0.54 g, 5.39 mmol, 5 eq) were added and the mixture was stirred at room temperature for 1 h. The resulting mixture was poured into water (100 mL) and extracted with DCM (3×100 mL). The combined organic extracts were dried over Na₂SO₄ and concentrated under vacuum. The crude material was purified by preparative HPLC (conditions below) to yield the title compound as a white solid (0.085 g, Yield: 14.76%).

¹H NMR (DMSO-d6, 400 MHz): δ 2.20 (s, 6H), 2.51-2.67 (m, 4H), 3.60-3.76 (m, 4H), 3.82-4.02 (m, 6H), 4.32-4.40 (m, 1H), 4.54 (d, J=16 Hz, 1H), 5.02 (d, J=5.2 Hz, 1H), 5.12 (s, 2H), 6.01 (s, 2H), 6.55-6.57 (m, 1H), 6.72 (d, J=1.6 Hz, 1H), 7.20-7.31 (m, 5H), 10.3 (bs, 1H).

LCMS (Method A): 1.492 min, MS: ES+ 566.0 (M+1).

HPLC (Method D): 5.507 min.

Chromatographic separation and isolation were conducted with a SHIMADZU NEXERA PREP WITH LH-40 AUTO PURIFICATION SYSTEM. The column used was Waters Sunfire C18 OBD (250 mm×19 mm 5 μm) and the compounds were eluted with, Mobile Phase A: 0.05% hydrochloric acid in water, Mobile Phase B: Acetonitrile with a gradient of T=0.01 min (78% A, 22% B); gradient to T=21.00 min (78% A, 22% B); T=21.01 min (2% A, 98% B) gradient to T=23.00 min (2% A, 98% B); T=23.01 min (78% A, 22% B); gradient to T=27 min (78% A, 22% B); Flow rate=12 ml/min; analysis time 27 min.

### Example 22: (S)-(2-(Benzyloxy)-4-chloro-6-hydroxyphenyl)(6-(2-(dimethylamino)ethoxy)-8-((tetrahydrofuran-3-yl)amino)-3,4-dihydroisoquinolin-2(1H)-yl)methanone

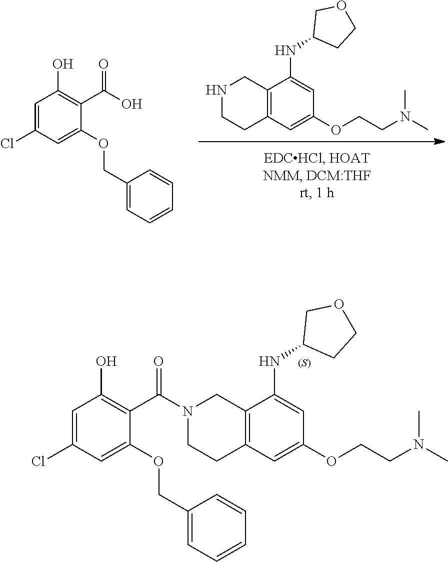

To a stirred solution of 2-(benzyloxy)-4-chloro-6-hydroxybenzoic acid (Intermediate F) (0.3 g, 1.07 mmol, 1 eq.) in DCM:THF (1:1, 8 mL) were added EDC·HCl (0.31 g, 1.61 mmol, 1.5 eq) and HOAT (0.146 g, 1.07 mmol, 1 eq) at room temperature. (S)-6-(2-(Dimethylamino)ethoxy)-N-(tetrahydrofuran-3-yl)-1,2,3,4-tetrahydroisoquinolin-8-amine hydrochloride (Intermediate MA) (0.39 g, 1.29 mmol, 1.2 eq) and N-methyl morpholine (0.54 g, 5.39 mmol, 5 eq) were added and the reaction mixture was stirred at room temperature for 1 h. The resulting mixture was poured into water (100 mL) and extracted with DCM (3×100 mL). The combined organic extracts were dried over Na₂SO₄, filtered and concentrated under vacuum. The crude material was purified by preparative HPLC (conditions below) to yield the title compound as a white solid. (0.075 g, Yield: 14.95%).

¹H NMR (DMSO-d6, 400 MHz): δ 1.98-1.99 (m, 1H), 2.33 (s, 6H), 2.57-2.68 (m, 4H), 3.60-3.74 (m, 3H), 3.82-4.01 (m, 6H), 4.02-4.56 (m, 2H), 5.03 (d, J=5.2 Hz, 1H), 5.12 (s, 2H), 6.02 (s, 2H), 6.55-6.57 (m, 1H), 6.72 (d, J=1.6 Hz, 1H), 7.22-7.33 (m, 5H), 10.3 (bs, 1H).

LCMS (Method A): 1.498 min, MS: ES+ 566.0 (M+1).

HPLC (Method D): 5.502 min.

Preparative HPLC: Chromatographic separation and isolation were conducted with a SHIMADZU NEXERA PREP WITH LH-40 AUTO PURIFICATION SYSTEM. The column used was Waters Sunfire C18 OBD (250 mm×19 mm 5 μm) and the compounds were eluted with, Mobile Phase A: 0.05% hydrochloric acid in water, Mobile Phase B: 30% A line in Acetonitrile with a gradient of T=0.01 min (72% A, 28% B); gradient to T=16.00 min (56% A, 44% B); T=16.01 min (2% A, 98% B) gradient to T=18.00 min (2% A, 98% B); T=18.01 min (72% A, 28% B); gradient to T=21 min (72% A, 28% B); Flow rate=12 mL/min; analysis time 21 mins.

### Example 23 (S)-(2-(Benzyloxy)-6-hydroxy-4-(trifluoromethyl) phenyl) (4-((tetrahydrofuran-3-yl) amino) isoindolin-2-yl) methanone

### Step 1: (S)-(2, 6-Dihydroxy-4-(trifluoromethyl) phenyl) (4-((tetrahydrofuran-3-yl) amino) isoindolin-2-yl) methanone

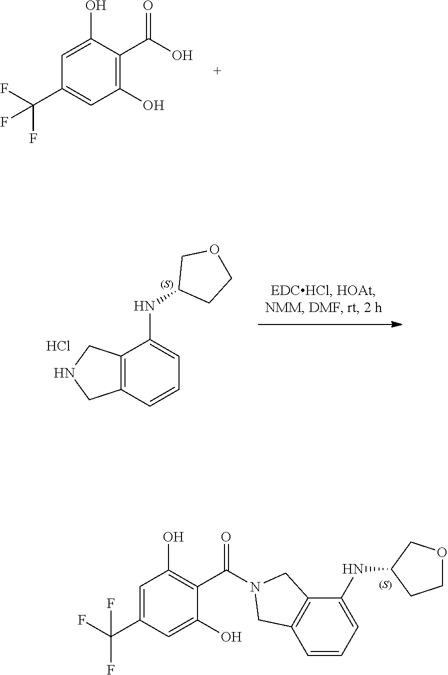

To a stirred solution of 2, 6-dihydroxy-4-(trifluoromethyl) benzoic acid (Intermediate ZA) (0.6 g, 2.70 mmol, 1.0 eq.) in DMF (6.0 mL) were added EDC·HCl (0.77 g, 4.05 mmol, 1.5 eq.), HOBT (0.036 g, 2.70 mmol, 1.0 eq.) (S)—N-(tetrahydrofuran-3-yl)isoindolin-4-amine hydrochloride (Intermediate A) (0.82 g, 4.05 mmol, 1.5 eq) and TEA (1.36 g, 13.5 mmol, 5.0 eq.) at room temperature. The mixture was heated to 120° C. using microwave radiation for 3 h. The resulting mixture was diluted with ice cold water (100 mL) and extracted with EtOAc (3×100 mL). The combined organic extracts were dried over Na₂SO₄, filtered and concentrated under vacuum. The crude material was purified by chromatography on silica (eluting with 3.8% MeOH in DCM) and dried under high vacuum yielding (S)-(2, 6-dihydroxy-4-(trifluoromethyl) phenyl) (4-((tetrahydrofuran-3-yl) amino) isoindolin-2-yl) methanone (0.24 g, Yield: 13.60%).

¹H NMR (DMSO-d6, 400 MHz) δ 1.76-1.87 (m, 1H), 2.12-2.21 (m, 1H), 2.18 (m, 2H), 2.71 (d, J=30.4 Hz, 3H), 3.18 (d, J=5.2 Hz, 2H), 3.42-4.14 (m, 3H), 4.36-4.70 (m, 2H), 6.34 (s, 1H), 6.44-6.67 (m, 3H), 7.08-7.20 (m, 2H), 10.32 (d, J=8.4 Hz, 2H), 15.13 (s, 1H). LCMS (Method N): 1.451 min, MS: ES+ 409.01 (M+1).

### Step 2: (S)-(2-(Benzyloxy)-6-hydroxy-4-(trifluoromethyl) phenyl) (4-((tetrahydrofuran-3-yl) amino) isoindolin-2-yl) methanone

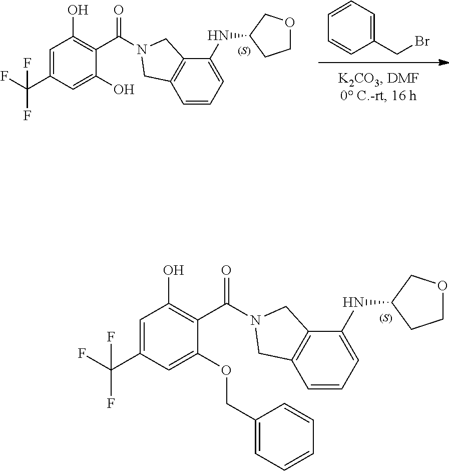

To a stirred solution of (S)-(2, 6-dihydroxy-4-(trifluoromethyl) phenyl) (4-((tetrahydrofuran-3-yl) amino) isoindolin-2-yl) methanone (step 1) (0.13 g, 0.31 mmol, 1.0 eq.) in DMF (1.0 mL) were added K₂CO₃ (0.131 g, 0.95 mmol, 3.0 eq.) followed by dropwise addition of benzyl bromide (0.0544 g, 0.318 mmol, 1.0 eq.) at 0° C. The reaction mixture was allowed to stir at room temperature for 16 h. The resulting mixture was diluted with ice cold water (50 mL) and extracted with EtOAc (3×50 mL). The combined organic extracts were dried over Na₂SO₄, filtered and concentrated under vacuum. The crude material was purified by chromatography on silica (eluting with 2.0% MeOH in DCM) and dried under high vacuum yielding (S)-(2-(benzyloxy)-6-hydroxy-4-(trifluoromethyl) phenyl) (4-((tetrahydrofuran-3-yl) amino) isoindolin-2-yl) methanone as a white solid (0.003 g, Yield: 3.15%).

¹H NMR (DMSO-d6, 400 MHz) δ 1.76-1.87 (m, 1H), 2.12-2.21 (m, 1H), 2.18-3.53 (m, 1H), 3.57-3.7 (m, 1H), 3.71-3.80 (m, 1H), 3.82-3.90 (m, 1H), 3.93-4.10 (m, 2H), 4.35-4.50 (m, 2H), 4.63-4.71 (m, 2H), 5.23 (d, J=3.2 Hz, 2H), 5.36-5.50 (m, 1H), 6.44-6.63 (m, 2H), 6.86 (d, J=4.8 Hz, 1H), 6.97 (s, 1H), 7.06-7.14 (m, 1H), 7.24-7.34 (m, 5H), 10.60 (s, 1H).

LCMS (Method V): 7.853 min, MS: ES+ 499.40 (M+1).

HPLC (Method I): 7.30 min.

Chromatographic separation and isolation were conducted with a WATERS 2545 BINARY PUMP WITH WATERS 2489 UV DETECTOR WITH WATERS ACQUITY QDA. The column (PCH-020) used was Waters Sunfire C18 OBD (250 mm×19 mm 5 μm) and the compounds were eluted with, Mobile Phase A: 0.05% FORMIC ACID IN WATER, Mobile Phase B: Acetonitrile with a gradient of T=0.01 min (50% A, 50% B); gradient to T=16.00 min (25% A, 75% B); T=16.01 min (0% A, 100% B); gradient to T=18.00 min (0% A, 100% B); T=18.01 min (50% A, 50% B); gradient to T=20.00 min (100% A, 50% B); Flow rate=22 ml/min; analysis time 01.27 min.

### Example 24 (S)-(2-(Benzyloxy)-4-(difluoromethyl)-6-hydroxyphenyl)(6-(2-(dimethylamino)ethoxy)-8-((tetrahydrofuran-3-yl)amino)-3,4-dihydroisoquinolin-2(1H)-yl)methanone

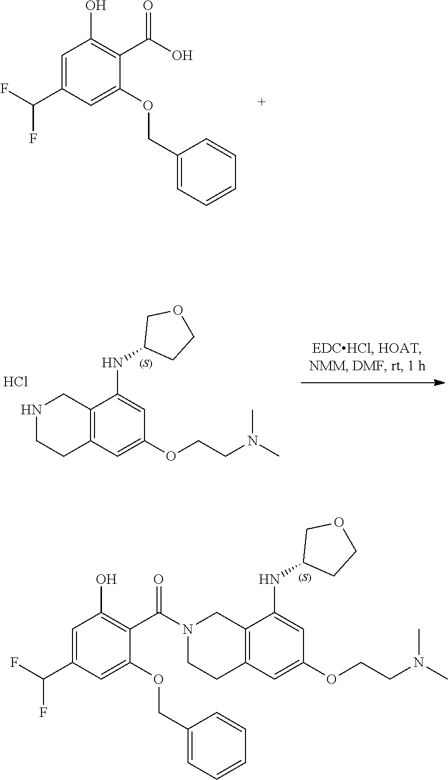

To a stirred solution of 2-(benzyloxy)-4-(difluoromethyl)-6-hydroxybenzoic acid (Intermediate D) (0.12 g, 0.408 mmol, 1 eq.) in DMF (1.2 mL) were added EDC·HCl (0.117 g, 0.612 mmol, 1.5 eq) and HOAT (0.055 g, 0.408 mmol, 1 eq) at room temperature. (S)-6-(2-(Dimethylamino)ethoxy)-N-(tetrahydrofuran-3-yl)-1,2,3,4-tetrahydroisoquinolin-8-amine hydrochloride (Intermediate MA) (0.167 g, 0.48 mmol, 1.2 eq) and NMM (0.20 g, 2.04 mmol, 5 eq) were added and the reaction mixture was stirred at room temperature for 1 h. The resulting mixture was poured into water (50 mL) and extracted into DCM (3×50 mL). The combined organic extracts were dried over Na₂SO₄, filtered and concentrated under vacuum. The crude material was purified by preparative HPLC (conditions below) followed by pure fraction lyophilization yielding (S)-(2-(benzyloxy)-4-(difluoromethyl)-6-hydroxyphenyl)(6-(2-(dimethylamino)ethoxy)-8-((tetrahydrofuran-3-yl)amino)-3,4-dihydroisoquinolin-2(1H)-yl)methanone as a white solid (0.068 g, Yield: 29.5%).

1H NMR (DMSO-d6, 400 MHz): δ 1.98-1.99 (m, 1H), 2.33 (s, 6H), 2.54-2.66 (m, 4H), 3.61-3.75 (m, 3H), 3.85-4.03 (m, 6H), 4.58-5.03 (m, 2H), 5.14 (s, 2H), 6.02 (s, 2H), 6.70-6.77 (m, 2H), 6.81 (d, J=7.2 Hz, 1H), 6.94 (s, br, 1H), 7.21-7.35 (m, 5H), 10.25 (bs, 1H).

LCMS (Method A): 1.392 min, MS ES+: 582.2 (M+1).

HPLC (Method N): 5.482 min.

Chromatographic separation and isolation were conducted with a purification system with a WATERS 2545 QUARTENERY SYSTEM WITH WATERS 2489 UV detector. The column used was Waters Sunfire C18 OBD (250 mm×19 mm 5 μm) and the compounds were eluted with, Mobile Phase A: 0.05% HYDROCHLORIC ACID IN MERCK WATER, Mobile Phase B Acetonitrile:MeOH:IPA (65:25:10) with a gradient of T=0 min (75% A, 25% B); gradient to T=15.00 min (71% A, 29% B); T=24.00 min (71% A, 29% B) gradient to T=24.01 min (0% A, 100% B); T=27.00 min (0% A, 100% B); T=27.01 min (75% A, 25% B); T=30.00 min (75% A, 25% B); Flow rate=9 ml/min; analysis time 30 min.

### Example 25 (R)-(2-(Benzyloxy)-4-(difluoromethyl)-6-hydroxyphenyl)(6-(2-(dimethylamino)ethoxy)-8-((tetrahydrofuran-3-yl)amino)-3,4-dihydroisoquinolin-2(1H)-yl)methanone

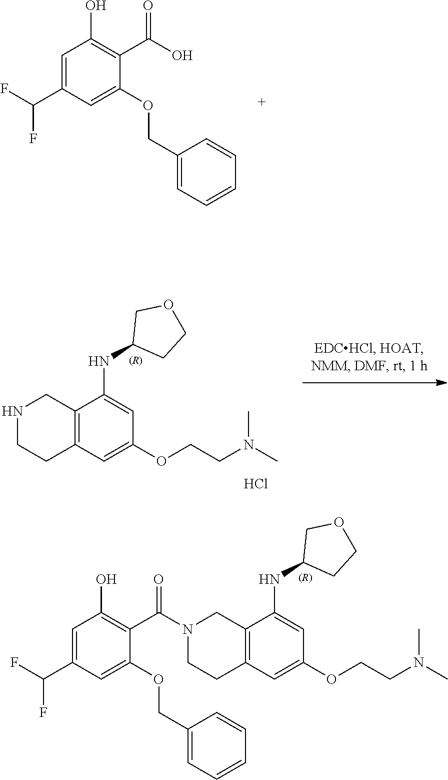

To a stirred solution of 2-(benzyloxy)-4-(difluoromethyl)-6-hydroxybenzoic acid (Intermediate D) (0.12 g, 0.408 mmol, 1 eq.) in DMF (1.2 mL) were added EDC·HCl (0.117 g, 0.61 mmol, 1.5 eq) and HOAT (0.055 g, 0.408 mmol, 1 eq) at room temperature. (R)-6-(2-(Dimethylamino)ethoxy)-N-(tetrahydrofuran-3-yl)-1,2,3,4-tetrahydroisoquinolin-8-amine hydrochloride (Intermediate M) (0.167 g, 0.489 mmol, 1.2 eq) and NMM (0.2 g, 2.04 mmol, 5 eq) were added and the reaction mixture was stirred at room temperature for 1 h. The resulting mixture was poured into water (50 mL) and extracted into DCM (3×50 mL). The combined organic extracts were dried over Na₂SO₄, filtered and concentrated under vacuum. The crude material was purified by preparative HPLC (conditions below) followed by pure fraction lyophilization yielding (R)-(2-(benzyloxy)-4-(difluoromethyl)-6-hydroxyphenyl)(6-(2-(dimethylamino)ethoxy)-8-((tetrahydrofuran-3-yl)amino)-3,4-dihydroisoquinolin-2(1H)-yl)methanone as a white solid (0.095 g, Yield: 37.11%).

¹H NMR (DMSO-d6, 400 MHz): δ 1.98-1.99 (m, 1H), 2.45 (s, 6H), 2.63-2.68 (m, 2H), 2.92 (bs, 2H), 3.31-3.38 (m, 2H), 3.61-3.77 (m, 2H), 3.85-3.95 (m, 2H), 4.01-4.09 (m, 3H), 4.39-4.58 (m, 2H), 5.08 (d, J=5.2 Hz, 1H), 5.14 (s, 2H), 6.04 (s, br, 2H), 6.32 (bs, 1H), 6.56 (bs, 1H), 6.72-6.78 (m, 2H), 6.81 (d, J=7.2 Hz, 1H), 7.08 (s, 1H), 7.20-7.34 (m, 5H), 10.25 (bs, 1H).

LCMS (Method A): 1.403 min, MS ES+: 582.1 (M+1).

HPLC (Method N): 5.491 min.

Chromatographic separation and isolation were conducted with water 2545 purification system with UV detector. The column used was Shim-Pack GIST C18 (250 mm×20 mm×5 μm) and the compounds were eluted with, Mobile Phase A: 0.05% HYDROCHLORIC ACID IN MERCK WATER, Mobile Phase B: Acetonitrile: MeOH:IPA (65:25:10) with a gradient of T=0 min (75% A, 25% B); gradient to T=15.00 min (71% A, 29% B); T=24.00 min (71% A, 29% B) gradient to T=24.01 min (0% A, 100% B); T=27.00 min (0% A, 100% B); T=27.01 min (75% A, 25% B); T=30.00 min (75% A, 25% B); Flow rate=9 ml/min; analysis time 30 min.

### Example 26 (2-(Benzyloxy)-4-(difluoromethyl)-6-hydroxyphenyl)(5-(piperidin-4-yloxy)isoindolin-2-yl)methanone

### Step 1: tert-Butyl 5-((1-benzylpiperidin-4-yl) oxy) isoindoline-2-carboxylate

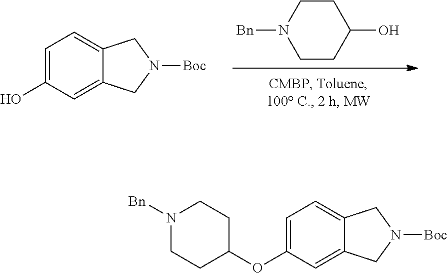 [SMILES: CC(C)(C)OC(=O)N1Cc2ccc(O)cc2C1 ; OC1CCN(Cc2ccccc2)CC1 ; CC(C)(C)OC(=O)N1Cc2ccc(OC3CCN(Cc4ccccc4)CC3)cc2C1]

Performed in 3 parallel batches: To a stirred solution of tert-butyl 5-hydroxyisoindoline-2-carboxylate (Intermediate V step 2) (0.5 g, 2.125 mmol, 1.0 eq.) and 1-benzylpiperidin-4-ol (0.4 g, 2.125 mmol, 1.0 eq) (CAS: 4727-72-4) in toluene (5.0 ml) was added dropwise CMBP (1.024 g, 4.25 mmol, 2.0 eq) at room temperature. The resulting mixture was heated to 120° C. using microwave radiation for 2 h. The resulting mixture was diluted with water (50 mL) and extracted with EtOAc (3×50 mL). The combined organic extracts were dried over Na₂SO₄, filtered and concentrated under vacuum. The crude material was purified by chromatography on silica (eluting with 12% EtOAc in hexane) yielding tert-butyl 5-((1-benzylpiperidin-4-yl) oxy) isoindoline-2-carboxylate as a white solid material. (1.8 g, Yield: 69.11%).

¹H NMR (DMSO-d6, 400 MHz): δ 1.45 (s, 9H), 1.57-1.64 (m, 2H), 1.90-1.93 (m, 2H), 2.19-2.24 (m, 2H), 2.67-2.68 (m, 2H), 3.48 (s, 2H), 4.33-4.34 (m, 1H), 4.50 (t, J=13.2 Hz, 1H), 6.85 (d, J=8.4 Hz, 1H), 6.92 (d, J=6.4 Hz, 1H), 7.17-7.31 (m, 6H),

LCMS (Method A): 1.463 min, MS: ES+ 409.1 (M+1).

### Step 2: tert-Butyl 5-(piperidin-4-yloxy) isoindoline-2-carboxylate

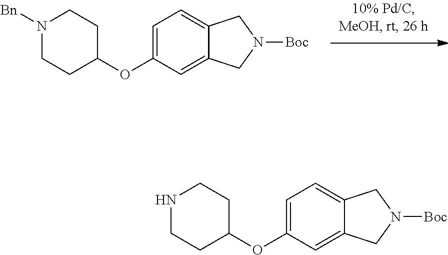 [SMILES: CC(C)(C)OC(=O)N1Cc2ccc(OC3CCN(Cc4ccccc4)CC3)cc2C1 ; CC(C)(C)OC(=O)N1Cc2ccc(OC3CCNCC3)cc2C1]

To a stirred solution of tert-butyl 5-((1-benzylpiperidin-4-yl) oxy) isoindoline-2-carboxylate (step 1) (1.8 g, 4.401 mmol, 1.0 eq) in MeOH (18 mL) was added 10% Pd/C (85% w/w) and the reaction mixture was stirred under an atmosphere of hydrogen at room temperature for 26 h. The resulting mixture was filtered through Celite® (filter material) and washed with MeOH (250 mL). The filtrate was concentrated under vacuum yielding tert-butyl 5-(piperidin-4-yloxy) isoindoline-2-carboxylate as a colourless liquid (1.3 g, Yield: 92.66%).

¹H NMR (DMSO-d6, 400 MHz): δ 1.34-1.43 (m, 2H), 1.45 (s, 9H), 1.88-1.90 (m, 2H), 2.54-2.59 (m, 2H), 2.91-2.94 (m, 2H), 3.43 (s, 1H), 3.57 (s, 1H), 4.32-4.35 (m, 1H), 4.51 (t, J=12.4 Hz, J=14 Hz, 4H), 6.82 (d, J=8.4 Hz, 1H), 6.92 (d, J=4.8 Hz, 1H), 7.17-7.20 (m, 1H).

LCMS (Method A): 1.274 min, MS ES+: 319.1 (M+1).

### Step 3: tert-Butyl 5-((1-(((9H-fluoren-9-yl)methoxy)carbonyl)piperidin-4-yl)oxy)isoindoline-2-carboxylate

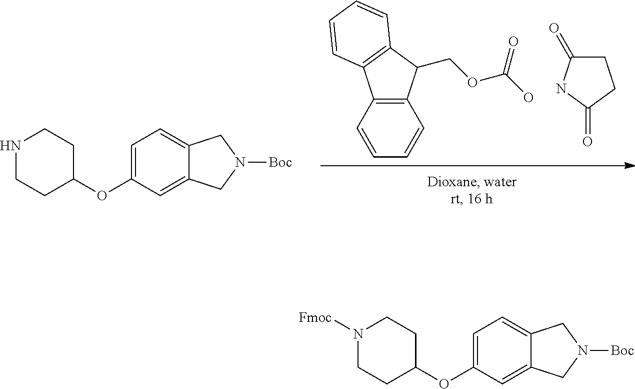

To a stirred solution of tert-butyl 5-(piperidin-4-yloxy) isoindoline-2-carboxylate (step 2) (1.3 g, 4.08 mmol, 1.0 eq) in 1,4-dioxane:water (1:1, 46.8 mL) was added (9H-fluoren-9-yl)methyl(2,5-dioxopyrrolidin-1-yl)carbonate (CAS: 5382-16-1) (1.37 g, 4.08 mmol, 1.0 eq) at room temperature. The reaction mixture was stirred at room temperature for 16 h. The resulting mixture was diluted with water (100 mL) and extracted with EtOAc (3×100 mL). The combined organic extracts were dried over Na₂SO₄, filtered and concentrated under vacuum. The crude material was purified by chromatography on silica (eluting with 15% EtOAc in Hexane) yielding tert-butyl 5-((1-(((9H-fluoren-9-yl)methoxy)carbonyl)piperidin-4-yl)oxy)isoindoline-2-carboxylate as a white solid (1.0 g, Yield: 45.30%).

¹H NMR (DMSO-d6, 400 MHz): δ 1.45 (s, 9H), 1.83 (bs, 2H), 3.15-3.20 (m, 2H), 3.60 (bs, 2H), 4.28 (t, J=6 Hz, J=12.4 Hz, 1H), 4.40 (s, 2H), 4.52 (t, J=13.6 Hz, J=24.8 Hz, 5H), 6.88 (d, J=8 Hz, 1H), 6.97 (d, J=6.4 Hz, 1H), 7.20-7.23 (m, 1H), 7.33-7.44 (m, 4H), 7.64 (d, J=7.6 Hz, 2H), 7.90 (d, J=7.6 Hz, 2H).

LCMS (Method A): 2.553 min, MS ES+: 441 (M−100).

### Step 4: (9H-Fluoren-9-yl) methyl 4-(isoindolin-5-yloxy) piperidine-1-carboxylate hydrochloride

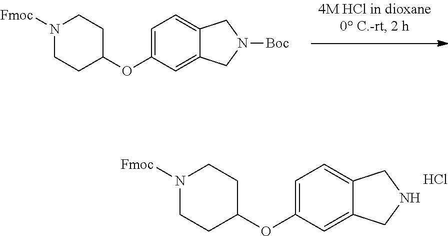

To a stirred solution of tert-butyl 5-((1-(((9H-fluoren-9-yl) methoxy) carbonyl) piperidin-4-yl) oxy) isoindoline-2-carboxylate (step 3) (1.0 g, 1.85 mmol, 1.0 eq) in DCM (10 mL) cooled to 0° C. was added 4M HCl in dioxane (5 mL). The reaction mixture was stirred at room temperature for 2 h. The resulting mixture was concentrated under vacuum and the crude material was triturated with n-pentane (60 mL) yielding (9H-fluoren-9-yl) methyl 4-(isoindolin-5-yloxy) piperidine-1-carboxylate hydrochloride as a light brown solid. (0.99 g, Yield: quantitative).

¹H NMR (DMSO-d6, 400 MHz): 1.43 (bs, 2H), 1.83 (bs, 2H), 3.15-3.20 (m, 2H), 3.57 (s, 6H), 4.27-4.55 (m, 8H), 6.95-6.98 (m, 2H), 7.28-7.44 (m, 5H), 7.63 (d, J=7.6 Hz, 2H), 7.90 (d, J=7.6 Hz, 2H), 9.51 (s, 2H).

LCMS (Method A): 1.624 min, MS ES+: 441.1 (M+1).

### Step 5: (9H-Fluoren-9-yl)methyl4-((2-(2-(benzyloxy)-4-(difluoromethyl)-6-hydroxybenzoyl)isoindolin-5-yl)oxy)piperidine-1-carboxylate

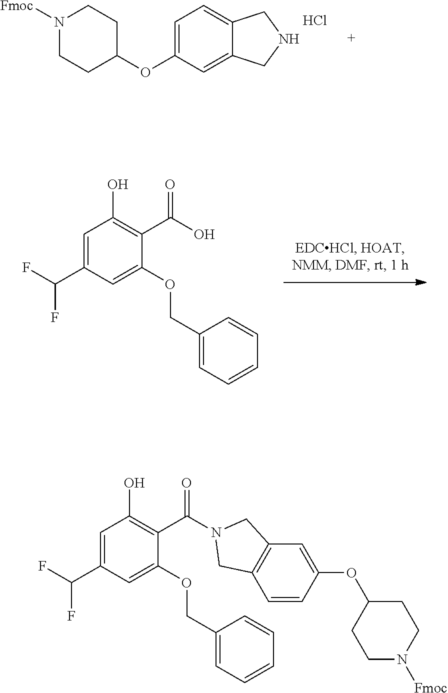

Performed in 5 parallel batches, each of 0.12 g scale: To a stirred solution of 2-(benzyloxy)-4-(difluoromethyl)-6-hydroxybenzoic acid (Intermediate 0) (0.12 g, 0.408 mmol, 1.0 eq) in DMF (1.5 mL) were added EDC·HCl (0.117 g, 0.612 mmol, 1.5 eq) and HOAT (0.055 g, 0.408 mmol, 1.0 eq) at room temperature and stirred for 15 mins. (9H-Fluoren-9-yl) methyl 4-(isoindolin-5-yloxy) piperidine-1-carboxylate hydrochloride (step 4) (0.215 g, 0.489 mmol, 1.2 eq) and NMM (0.206 g, 2.04 mmol, 5.0 eq) were added and the mixture was stirred at room temperature for 1 h. The resulting mixture was diluted with cold water (50 ml) and extracted with EtOAc (3×50 mL). The combined organic extracts were dried over Na₂SO₄, filtered and concentrated under vacuum. The crude material was purified by chromatography on silica (eluting with 3% MeOH in DCM) yielding (9H-fluoren-9-yl)methyl4-((2-(2-(benzyloxy)-4-(difluoromethyl)-6-hydroxybenzoyl)isoindolin-5-yl)oxy)piperidine-1-carboxylate as a light brown solid (0.75 g, Yield: 43.46%).

¹H NMR (DMSO-d6, 400 MHz): δ 1.40-1.60 (m, 2H), 1.83-1.85 (m, 2H), 3.11-3.21 (m, 3H), 3.60-3.61 (m, 2H), 4.11-4.73 (m, 8H), 5.17 (s, 2H), 6.74 (s, 1H), 6.81-7.16 (m, 4H), 7.24-7.65 (m, 11H), 7.84-7.91 (m, 2H), 10.06 (s, 2H).

LCMS (Method A): 2.276 min, MS ES+: 717.2 (M+1).

### Step 6: (2-(Benzyloxy)-4-(difluoromethyl)-6-hydroxyphenyl)(5-(piperidin-4-yloxy)isoindolin-2-yl)methanone

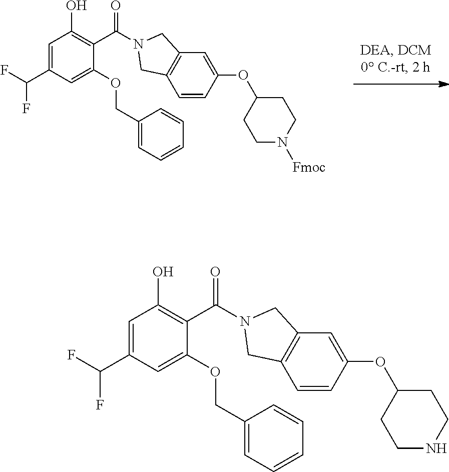

To a stirred solution of (9H-fluoren-9-yl)methyl 4-((2-(2-(benzyloxy)-4-(difluoromethyl)-6-hydroxybenzoyl)isoindolin-5-yl)oxy)piperidine-1-carboxylate (step 5) (0.3 g, 0.481 mmol, 1.0 eq) in DCM (3 mL) cooled to 0° C. was added diethyl amine (1.5 mL) and the reaction mixture was stirred at room temperature for 2 h. The resulting mixture was concentrated under vacuum and the crude material was purified by reverse phase column chromatography (eluting with 20% ACN in 0.05% FA in water) yielding (2-(benzyloxy)-4-(difluoromethyl)-6-hydroxyphenyl)(5-(piperidin-4-yloxy)isoindolin-2-yl)methanone as a white solid. (0.07 g, Yield: 34%).

¹H NMR (DMSO-d6, 400 MHz): δ 1.54-1.62 (m, 2H), 1.92-1.99 (m, 2H), 2.67-2.77 (m, 2H), 2.99-3.08 (m, 2H), 4.35-4.45 (m, 3H), 4.71-4.75 (m, 2H), 5.16 (s, 2H), 6.75-6.94 (m, 4H), 7.03-7.16 (m, 2H), 7.26-7.33 (m, 5H).

LCMS (Method U): 6.420 min, MS ES+: 495.3 (M+1).

HPLC (Method L): 6.420 min.

### Example 27: 3-(Benzyloxy)-4-(6-((dimethylamino)methyl)-2,3-dihydro-1H-pyrrolo[3,4-c]pyridine-2-carbonyl)-5-hydroxybenzonitrile

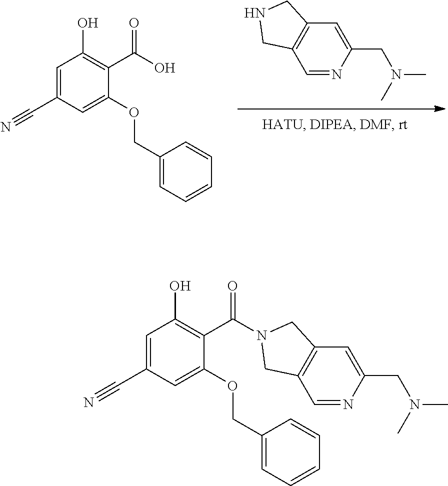 [SMILES: N#Cc1cc(O)c(C(=O)O)c(OCc2ccccc2)c1 ; CN(C)Cc1cc2c(cn1)CNC2 ; CN(C)Cc1cc2c(cn1)CN(C(=O)c1c(O)cc(C#N)cc1OCc1ccccc1)C2]

DIPEA (1.5 mL, 8.61 mmol, 58.0 eq) was added to a stirred mixture of 2-(benzyloxy)-4-cyano-6-hydroxybenzoic acid (Intermediate K) (40 mg, 0.15 mmol, 1.0 eq), 1-(2,3-dihydro-1H-pyrrolo[3,4-c]pyridin-6-yl)-N,N-dimethyl-methanamine dihydrobromide (Intermediate 3) (50 mg, 0.15 mmol, 1.0 eq) and HATU (68 mg, 0.18 mmol, 1.2 eq) in CM (2.0 mL). The mixture was stirred at room temperature overnight. The resulting mixture was diluted with sodium hydrogen carbonate (sat. aq., ca. 5 ml-) and filtered through a phase separator. The organic layer was concentrated under vacuum and the residue was purified by preparative HPLC (Prep-LC-9) to give (benzyloxy)-4-(6-((dimethylamino)methyl)-2,3-dihydro-1H-pyrrolo[3,4-c]pyridine-2-carbonyl)-5-hydroxybenzonitrile (16 mg, 25%) as an off-white solid.

¹H NMR (400 MHz, DMSO) b 2.15 (s, 3H), 2.19 (s, 3H), 3.53-3.48 (m, 2H), 4.54-4.37 (m, 2H), 4.79 (s, 2H), 5.19 (s, 2H), 6.89 (s, 1H), 7.16 (s, 1H), 7.32-7.22 (m, 5.5H), 7.47 (s, 0.5H), 8.36 (3, 0.5H), 8.51 (s, 0.5H). Exchangeable OH too broad to report.

LMS (LC-Method 1): 2.86 mih MS: ES+ 429.5 (M+1).

The following compounds were prepared according to the method described above using the indicated intermediates.

| Ex. | Structure and Name | Analytical Data |
|---|---|---|
| 27.1 | 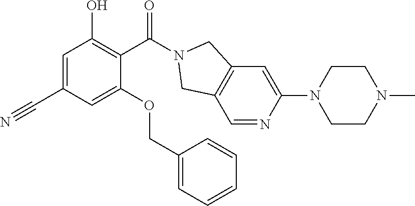 [SMILES: CN1CCN(c2cc3c(cn2)CN(C(=O)c2c(O)cc(C#N)cc2OCc2ccccc2)C3)CC1] | ¹H NMR (400 MHz, DMSO) δ 2.37- 2.28 (m, 3H), 3.52-3.42 (m, 4H), 4.38- 4.29 (m, 2H), 4.71-4.68 (m, 2H), 5.19 (s, 2H), 6.72 (s, 0.5H), 6.91-6.87 (m, 0.5H), 7.18 (s, 2H), 7.32-7.26 (m, 5H), 8.00 (s, 0.5H), 8.15-8.13 (m, 0.5H), 10.67 (br s, 1H). Some CH₂ signals obscured by DMSO signal. Splitting of signals observed due to presence of rotamers. LCMS (LC-Method 1): 2.90 min, MS: ES+ 470.3 (M + 1). |
| | 3-(Benzyloxy)-5-hydroxy-4-(6-(4- | |
| | methylpiperazin-1-yl)-2,3-dihydro-1H- | |
| | pyrrolo[3,4-c]pyridine-2- | |
| | carbonyl)benzonitrile | |
| | Prepared from 2-(benzyloxy)-4-cyano-6- | |
| | hydroxybenzoic acid (Intermediate K) | |
| | and | |
| | 6-(4-Methylpiperazin-1-yl)-2,3- | |
| | dihydro-1H-pyrrolo[3,4-c]pyridine | |
| | dihydrobromide (Intermediate B2-1) and | |
| | purified using prep Method prep-LC-2. | |
| 27.2 | 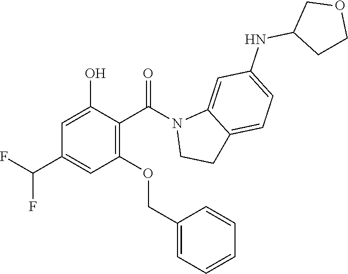 [SMILES: O=C(c1c(O)cc(C(F)F)cc1OCc1ccccc1)N1CCc2ccc(NC3CCOC3)cc21] | ¹H NMR (400 MHz, DMSO) δ 1.80- 1.62 (m, 1H), 2.18-2.07 (m, 1H), 2.92- 2.84 (m, 2H), 3.25-3.19 (m, 1H), 4.10-3.49 (m, 6H), 5.28-4.97 (m, 2H), 5.84-5.57 (m, 1H), 6.27-6.20 (m, 1H), 7.16-6.71 (m, 4H), 7.60- 7.25 (m, 5H), 10.56-10.03 (m, 1H). Splitting of signals observed due to presence of rotamers. LCMS (LC-Method 1): 4.61 min, MS: ES+ 481.6 (M + 1). |
| | (2-(Benzyloxy)-4-(difluoromethyl)-6- | |
| | hydroxyphenyl)(6-((tetrahydrofuran-3- | |
| | yl)amino)indolin-1-yl)methanone | |
| | Prepared from 2-(benzyloxy)-4- | |
| | (difluoromethyl)-6-hydroxybenzoic acid | |
| | (Intermediate D) and N- | |
| | (Tetrahydrofuran-3-yl)indolin-6-amine | |
| | dihydrochloride (Intermediate B1-2) | |
| | and | |
| | purified using prep method prep-LC-13. | |
| | Enantiomers were separated using SFC | |
| | Method SFC-5 to afford Ex 27.2a and | |
| | 27.2b as follows: | |
| 27.2a | 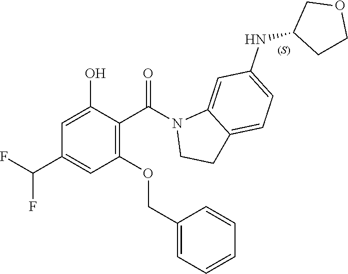 | LCMS (LC-Method 1): 4.62 min, MS: ES+ 481.5 (M + 1). |
| | (S)-(2-(Benzyloxy)-4-(difluoromethyl)-6- | |
| | hydroxyphenyl)(6-((tetrahydrofuran-3- | |
| | yl)amino)indolin-1-yl)methanone | |
| | or | |
| | 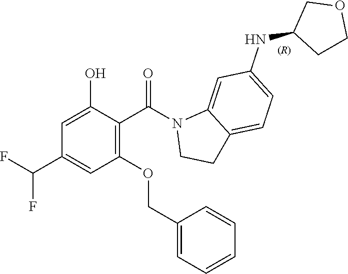 | |
| | (R)-(2-(Benzyloxy)-4-(difluoromethyl)-6- | |
| | hydroxyphenyl)(6-((tetrahydrofuran-3- | |
| | yl)amino)indolin-1-yl)methanone | |
| 27.2b | 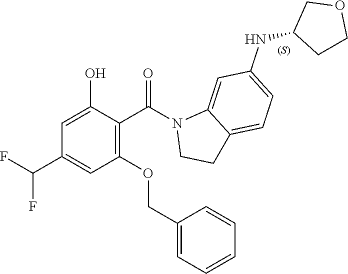 | LCMS (LC-Method 1): 4.62 min, MS: ES+ 481.6 (M + 1). |
| | (S)-(2-(Benzyloxy)-4-(difluoromethyl)-6- | |
| | hydroxyphenyl)(6-((tetrahydrofuran-3- | |
| | yl)amino)indolin-1-yl)methanone | |
| | or | |
| | 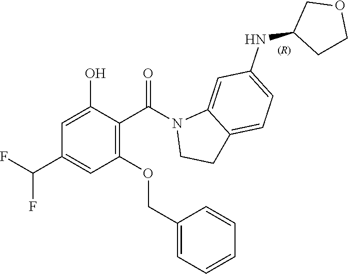 | |
| | (R)-(2-(benzyloxy)-4-(difluoromethyl)-6- | |
| | hydroxyphenyl)(6-((tetrahydrofuran-3- | |
| | yl)amino)indolin-1-yl)methanone | |
| 27.3 | 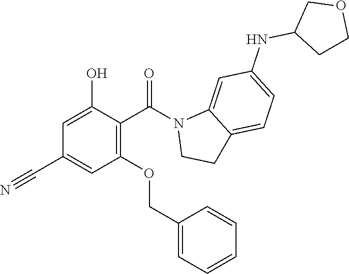 [SMILES: N#Cc1cc(O)c(C(=O)N2CCc3ccc(NC4CCOC4)cc32)c(OCc2ccccc2)c1] | ¹H NMR (400 MHz, DMSO) δ 1.81- 1.64 (m, 1H), 2.20-2.07 (m, 1H), 2.94- 2.85 (m, 2H), 4.11-3.50 (m, 7H), 5.27-5.01 (m, 2H), 5.87-5.62 (m, 1H), 6.29-6.23 (m, 1H), 6.96-6.88 (m, 2H), 7.13 (d, J = 7.2 Hz, 1H), 7.38- 7.24 (m, 4H), 7.57 (d, J = 1.6 Hz, 1H). Splitting of signals observed due to presence of rotamers. LCMS (LC- Method 1): 4.49 min, MS: ES+ 456.5 (M + 1). |
| | 3-(Benzyloxy)-5-hydroxy-4-(6- | |
| | ((tetrahydrofuran-3-yl)amino)indoline-1- | |
| | carbonyl)benzonitrile | |
| | Prepared from 2-(benzyloxy)-4-cyano-6- | |
| | hydroxybenzoic acid (Intermediate K) | |
| | and | |
| | N-(tetrahydrofuran-3-yl)indolin-6- | |
| | amine dihydrochloride | |
| | (Intermediate B1-2) and purified using | |
| | prep method SFC-2. | |
| 27.4 | 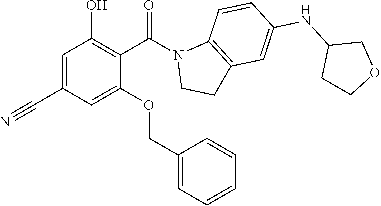 [SMILES: N#Cc1cc(O)c(C(=O)N2CCc3cc(NC4CCOC4)ccc32)c(OCc2ccccc2)c1] | ¹H NMR (400 MHz, DMSO) δ 1.78- 1.67 (m, 1H), 2.20-2.03 (m, 1H), 3.00- 2.94 (m, 2H), 4.11-3.44 (m, 7H), 5.23-4.95 (m, 2H), 5.75-5.60 (m, 1H), 6.51-6.41 (m, 2H), 6.86 (t, J = 15.9 Hz, 1H), 7.15-7.07 (m, 1H), 7.38- 7.24 (m, 4H), 7.91-7.87 (m, 1H), 10.98-10.48 (m, 1H). Splitting of signals observed due to presence of rotamers. LCMS (LC-Method 1): 4.20 min, MS: ES+ 456.6 (M + 1). |
| | 3-(Benzyloxy)-5-hydroxy-4-(5- | |
| | ((tetrahydrofuran-3-yl)amino) indoline-1- | |
| | carbonyl)benzonitrile | |
| | Prepared from 2-(benzyloxy)-4-cyano-6- | |
| | hydroxybenzoic acid (Intermediate K) | |
| | and | |
| | N-(Tetrahydrofuran-3-yl)indolin-5- | |
| | amine dihydrochloride (Intermediate B1- | |
| | 1) and purified using prep method SFC- | |
| | 1. | |
| 27.5 | 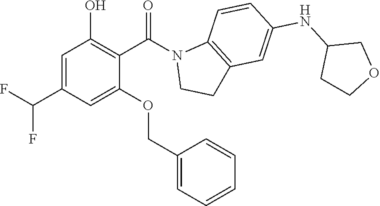 [SMILES: O=C(c1c(O)cc(C(F)F)cc1OCc1ccccc1)N1CCc2cc(NC3CCOC3)ccc21] | ¹H NMR (400 MHz, DMSO) δ 1.78- 1.64 (m, 1H), 2.21-2.06 (m, 1H), 2.97- 2.91 (m, 2H), 4.08-3.44 (m, 7H), 5.19- 4.91 (m, 2H), 5.55-5.52 (m, 1H), 5.75- 5.63 (m, 1H), 7.45-6.39 (m, 9H), 7.93- 7.89 (m, 1H), 10.30-10.18 (m, 1H). Splitting of signals observed due to presence of rotamers. LCMS (LC- Method 1): 4.36 min, MS: ES+ 481.6 (M + 1). |
| | (2-(Benzyloxy)-4-(difluoromethyl)-6- | |
| | hydroxyphenyl)(5-((tetrahydrofuran-3- | |
| | yl)amino)indolin-1-yl)methanone | |
| | Prepared from 2-(benzyloxy)-4- | |
| | (difluoromethyl)-6-hydroxybenzoic acid | |
| | (Intermediate D) and N- | |
| | (Tetrahydrofuran-3-yl)indolin-5-amine | |
| | dihydrochloride (Intermediate B1-1) | |
| | and | |
| | purified using prep method SFC-1 | |
| | Enantiomers were separated using prep | |
| | Method SFC-6 to afford Ex 27.5a and | |
| | 27.5b | |
| 27.5a | 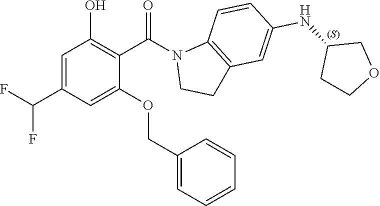 | LCMS (LC-Method 1): 4.35 min, MS: ES+ 481.6 (M + 1). |
| | (S)-(2-(Benzyloxy)-4-(difluoromethyl)-6- | |
| | hydroxyphenyl)(5-((tetrahydrofuran-3- | |
| | yl)amino)indolin-1-yl)methanone | |
| | or | |
| | 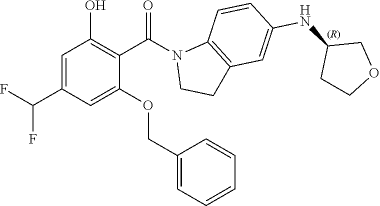 | |
| | (R)-(2-(Benzyloxy)-4-(difluoromethyl)-6- | |
| | hydroxyphenyl)(5-((tetrahydrofuran-3- | |
| | yl)amino)indolin-1-yl)methanone | |
| 27.5b | 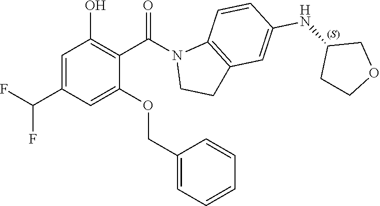 | LCMS (LC-Method 1): 4.36 min, MS: ES+ 481.4 (M + 1) |
| | (S)-(2-(Benzyloxy)-4-(difluoromethyl)-6- | |
| | hydroxyphenyl)(5-((tetrahydrofuran-3- | |
| | yl)amino)indolin-1-yl)methanone | |
| | or | |
| | 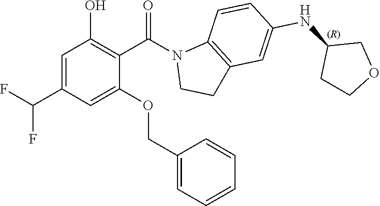 | |
| | (R)-(2-(Benzyloxy)-4-(difluoromethyl)-6- | |
| | hydroxyphenyl)(5-((tetrahydrofuran-3- | |
| | yl)amino)indolin-1-yl)methanone | |
| 27.6 | 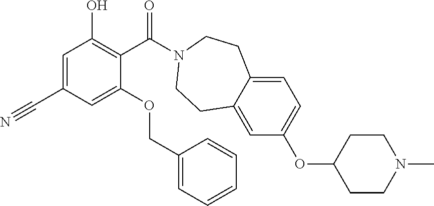 | ¹H NMR (400 MHz, DMSO) δ 1.64- 1.57 (m, 2H), 1.93-1.88 (m, 2H), 2.21- 2.16 (m, 5H), 2.61 (s, 2H), 2.81-2.67 (m, 5H), 3.58-3.48 (m, 2H), 3.88-3.79 (m, 1H), 4.32-4.24 (m, 1H), 5.18-5.16 (m, 2H), 7.15-6.63 (m, 5H), 7.36-7.25 (m, 5H), 8.16 (s, 1H, formate), 10.79- 10.45 (m, 1H). LCMS (LC-Method 1): 3.45 min, MS: ES+ 537.7 (M + 1). |
| | 3-(Benzyloxy)-5-hydroxy-4-(7-((1- | |
| | methylpiperidin-4-yl)oxy)-2,3,4,5- | |
| | tetrahydro-1H-benzo[d]azepine-3- | |
| | carbonyl)benzonitrile hydroformate | |
| | Prepared from 2-(benzyloxy)-4-cyano-6- | |
| | hydroxybenzoic acid (Intermediate K) | |
| | and | |
| | 7-((1-methylpiperidin-4-yl)oxy)- | |
| | 2,3,4,5-tetrahydro-1H-benzo[d]azepine | |
| | ditrifluoroacetate (Intermediate B4) and | |
| | purified using prep Method prep-LC-2 | |
| 27.7 | 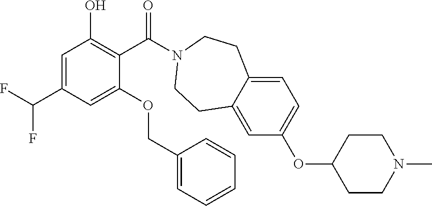 [SMILES: CN1CCC(Oc2ccc3c(c2)CCN(C(=O)c2c(O)cc(C(F)F)cc2OCc2ccccc2)CC3)CC1] | ¹H NMR (400 MHz, DMSO) δ 1.64- 1.54 (m, 2H), 1.92-1.88 (m, 2H), 2.17- 2.13 (m, 5H), 2.63-2.53 (m, 2H), 2.81- 2.66 (m, 4H), 3.27-3.17 (m, 2H), 3.54- 3.47 (m, 1H), 3.90-3.82 (m, 1H), 4.31- 4.23 (m, 1H), 5.11 (s, 2H), 6.77-6.61 (m, 4H), 6.94-6.87 (m, 1H), 7.06-7.01 (m, 1H), 7.38-7.24 (m, 5H). LCMS (LC-Method 1): 3.45 min, MS: ES+ 537.7 (M + 1). |
| | (2-(Benzyloxy)-4-(difluoromethyl)-6- | |
| | hydroxyphenyl)(7-((1-methylpiperidin-4- | |
| | yl)oxy)-1,2,4,5-tetrahydro-3H- | |
| | benzo[d]azepin-3-yl)methanone | |
| | Prepared from 2-(benzyloxy)-4- | |
| | (difluoromethyl)-6-hydroxybenzoic acid | |
| | (Intermediate D) and 7-((1- | |
| | methylpiperidin-4-yl)oxy)-2,3,4,5- | |
| | tetrahydro-1H-benzo[d]azepine | |
| | ditrifluoroacetate (Intermediate B4) and | |
| | purified using prep Method prep-LC-2 | |
| 27.8 | 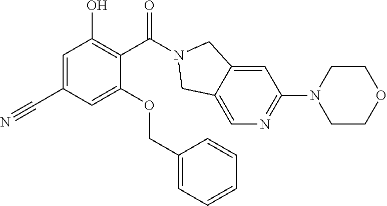 [SMILES: N#Cc1cc(O)c(C(=O)N2Cc3cnc(N4CCOCC4)cc3C2)c(OCc2ccccc2)c1] | ¹H NMR (400 MHz, DMSO) δ 3.45- 3.34 (m, 4H), 3.72-3.65 (m, 4H), 4.38- 4.32 (m, 2H), 4.72-4.69 (m, 2H), 5.21- 5.19 (m, 2H), 6.94-6.70 (m, 2H), 7.17 (s, 1H), 7.32-7.26 (m, 5H), 8.18-8.01 (m, 1H), 11.02-10.66 (m, 1H). LCMS (LC-Method 1): 3.25 min, MS: ES+ 457.3 (M + 1). |
| | 3-(Benzyloxy)-5-hydroxy-4-(6- | |
| | morpholino-2,3-dihydro-1H-pyrrolo[3,4- | |
| | c]pyridine-2-carbonyl)benzonitrile | |
| | Prepared from 2-(benzyloxy)-4-cyano-6- | |
| | hydroxybenzoic acid (Intermediate K) | |
| | and 4-(2,3-dihydro-1H-pyrrolo[3,4- | |
| | c]pyridin-6-yl)morpholine hydrobromide | |
| | (Intermediate B2-2) and purified using | |
| | prep Method prep-LC-3 | |
| 27.9 | 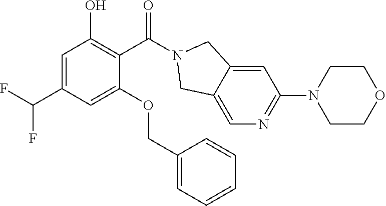 [SMILES: O=C(c1c(O)cc(C(F)F)cc1OCc1ccccc1)N1Cc2cnc(N3CCOCC3)cc2C1] | ¹H NMR (400 MHz, DMSO) δ 3.45- 3.36 (m, 4H), 3.72-3.65 (m, 4H), 4.46- 4.30 (m, 2H), 4.72-4.67 (m, 2H), 5.17 (s, 2H), 7.11-6.72 (m, 4H), 7.33-7.24 (m, 5H), 8.19-8.01 (m, 1H), 10.30- 10.26 (m, 1H). LCMS (LC-Method 1): 3.43 min, MS: ES+ 482.4 (M + 1). |
| | (2-(Benzyloxy)-4-(difluoromethyl)-6- | |
| | hydroxyphenyl)(6-morpholino-1,3- | |
| | dihydro-2H-pyrrolo[3,4-c]pyridin-2- | |
| | yl)methanone | |
| | Prepared from 2-(benzyloxy)-4- | |
| | (difluoromethyl)-6-hydroxybenzoic acid | |
| | (Intermediate D) and 4-(2,3-dihydro-1H- | |
| | pyrrolo[3,4-c]pyridin-6-yl)morpholine | |
| | hydrobromide (Intermediate B2-2) and | |
| | purified using prep Method prep-LC-5 | |
| 27.10 | 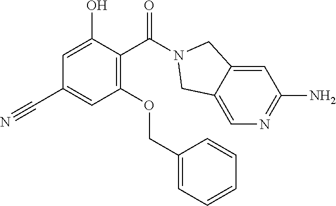 [SMILES: N#Cc1cc(O)c(C(=O)N2Cc3cnc(N)cc3C2)c(OCc2ccccc2)c1] | ¹H NMR (400 MHz, DMSO) δ 4.39- 4.20 (m, 2H), 4.63 (d, J = 8.0 Hz, 2H), 5.20 (s, 2H), 5.90 (d, J = 13.6 Hz, 2H), 6.43-6.29 (m, 1H), 6.72-6.58 (m, 1H), 6.91 (t, J = 1.3 Hz, 1H), 7.16-7.14 (m, 1H), 7.33-7.25 (m, 5H), 7.94-7.79 (m, 1H), 8.31 (s, 1H). LCMS (LC-Method 1): 2.80 min, MS: ES+ 387.4 (M + 1). |
| | 4-(6-Amino-2,3-dihydro-1H-pyrrolo[3,4- | |
| | c]pyridine-2-carbonyl)-3-(benzyloxy)-5- | |
| | hydroxybenzonitrile hydroformate | |
| | Prepared from 2-(benzyloxy)-4-cyano-6- | |
| | hydroxybenzoic acid (Intermediate K) | |
| | and | |
| | 2,3-dihydro-1H-pyrrolo[3,4- | |
| | c]pyridin-6-amine dihydrochloride | |
| | (Intermediate B6) and purified using | |
| | prep Method prep-LC-3 | |
| 27.11 | 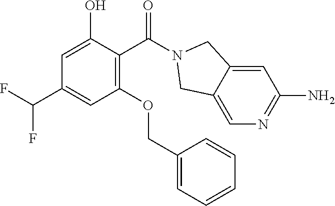 [SMILES: Nc1cc2c(cn1)CN(C(=O)c1c(O)cc(C(F)F)cc1OCc1ccccc1)C2] | ¹H NMR (400 MHz, DMSO) δ 4.36- 4.26 (m, 2H), 4.62 (d, J = 6.1 Hz, 2H), 5.16 (s, 2H), 5.89 (d, J = 13.6 Hz, 2H), 6.44-6.31 (m, 1H), 7.09-6.73 (m, 3H), 7.34-7.23 (m, 5H), 7.94-7.80 (m, 1H), 8.24 (s, 0.5H), 10.49-10.14 (m, 1H). LCMS (LC-Method 1): 3.01 min, MS: ES+ 412.4 (M + 1). |
| | (6-Amino-1,3-dihydro-2H-pyrrolo[3,4- | |
| | c]pyridin-2-yl)(2-(benzyloxy)-4- | |
| | (difluoromethyl)-6- | |
| | hydroxyphenyl)methanone | |
| | hydroformate | |
| | Prepared from 2-(benzyloxy)-4- | |
| | (difluoromethyl)-6-hydroxybenzoic acid | |
| | (Intermediate D) and 2,3-dihydro-1H- | |
| | pyrrolo[3,4-c]pyridin-6-amine | |
| | dihydrochloride (Intermediate B6) and | |
| | purified using Method prep-LC-3 | |
| 27.12 | 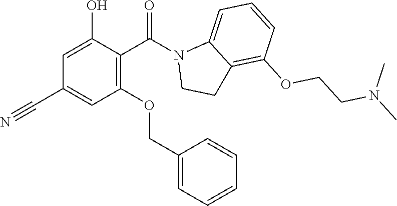 [SMILES: CN(C)CCOc1cccc2c1CCN2C(=O)c1c(O)cc(C#N)cc1OCc1ccccc1] | ¹H NMR (400 MHz, DMSO) δ 2.22 (d, J = 4.1 Hz, 6H), 2.68-2.59 (m, 2H), 2.98-2.86 (m, 2H), 3.79-3.62 (m, 2H), 4.11-4.07 (m, 2H), 5.21-4.94 (m, 2H), 7.35-6.59 (m, 10H), 8.40 (s, 1H). Splitting of signals observed due to presence of rotamers. LCMS (LC-Method 1): 3.27 min, MS: ES+ 458.5 (M + 1). |
| | 3-(Benzyloxy)-4-(4-(2- | |
| | (dimethylamino)ethoxy)indoline-1- | |
| | carbonyl)-5-hydroxybenzonitrile | |
| | Prepared from 2-(benzyloxy)-4-cyano-6- | |
| | hydroxybenzoic acid (Intermediate K) | |
| | and | |
| | 2-(Indolin-4-yloxy)-N,N- | |
| | dimethylethan-1-amine dihydrochloride | |
| | (Intermediate B5) and purified using | |
| | prep Method prep-LC-3 | |
| 27.13 | 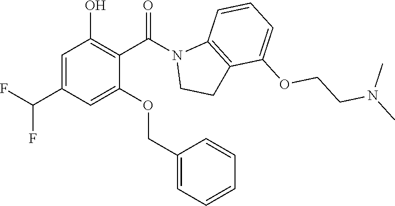 [SMILES: CN(C)CCOc1cccc2c1CCN2C(=O)c1c(O)cc(C(F)F)cc1OCc1ccccc1] | ¹H NMR (400 MHz, DMSO) δ 2.23- 2.21 (m, 6H), 2.67-2.60 (m, 2H), 2.99- 2.83 (m, 2H), 3.80-3.64 (m, 2H), 4.12- 4.07 (m, 2H), 5.18-4.92 (m, 2H), 7.79- 6.58 (m, 10H), 8.29 (s, 1H), 10.73- 10.32 (m, 1H). Splitting of signals observed due to presence of rotamers. LCMS (LC-Method 1): 3.40 min, MS: ES+ 483.5 (M + 1). |
| | (2-(Benzyloxy)-4-(difluoromethyl)-6- | |
| | hydroxyphenyl)(4-(2- | |
| | (dimethylamino)ethoxy)indolin-1- | |
| | yl)methanone | |
| | Prepared from 2-(benzyloxy)-4- | |
| | (difluoromethyl)-6-hydroxybenzoic acid | |
| | (Intermediate D) and 2-(Indolin-4-yloxy)- | |
| | N,N-dimethylethan-1-amine | |
| | dihydrochloride (Intermediate B5) and | |
| | purified using prep method prep-LC-3 | |
| 27.14 | 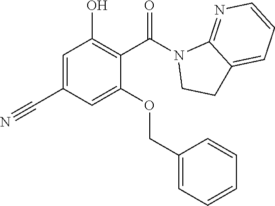 [SMILES: N#Cc1cc(O)c(C(=O)N2CCc3cccnc32)c(OCc2ccccc2)c1] | ¹H NMR (400 MHz, DMSO) δ 3.15- 2.88 (m, 2H), 4.12-4.06 (m, 2H), 4.93 (d, J = 12.5 Hz, 1H), 5.11 (d, J = 12.5 Hz, 1H), 6.91-6.82 (m, 2H), 7.09-7.00 (m, 3H), 7.27-7.23 (m, 3H), 7.62-7.59 (m, 1H), 7.74-7.71 (m, 1H), 10.30 (s, 1H). LCMS (LC-Method 1): 4.25 min, MS: ES+ 372.4 (M + 1). |
| | 3-(Benzyloxy)-4-(2,3-dihydro-1H- | |
| | pyrrolo[2,3-b]pyridine-1-carbonyl)-5- | |
| | hydroxybenzonitrile | |
| | Prepared from 2-(benzyloxy)-4-cyano-6- | |
| | hydroxybenzoic acid (Intermediate K) and | |
| | 2,3-dihydro-7-azaindole (CAS | |
| | 10592-27-5) and purified using prep | |
| | method prep-LC-3 | |
| 27.15 | 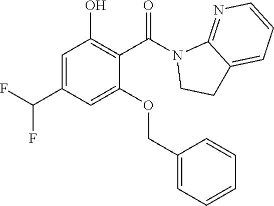 [SMILES: O=C(c1c(O)cc(C(F)F)cc1OCc1ccccc1)N1CCc2cccnc21] | ¹H NMR (400 MHz, DMSO) δ 3.15- 2.74 (m, 2H), 4.15-4.06 (m, 2H), 5.29- 4.84 (m, 2H), 8.18-6.61 (m, 11H), 9.99-9.84 (m, 1H). LCMS (LC-Method 1): 5.75 min, MS: ES+ 397.5 (M + 1). |
| | (2-(Benzyloxy)-4-(difluoromethyl)-6- | |
| | hydroxyphenyl)(2,3-dihydro-1H- | |
| | pyrrolo[2,3-b]pyridin-1-yl)methanone | |
| | Prepared from 2-(benzyloxy)-4- | |
| | (difluoromethyl)-6-hydroxybenzoic acid | |
| | (Intermediate D) and 2,3-dihydro-7- | |
| | azaindole (CAS 10592-27-5) and | |
| | purified using method prep-LC-9 | |
| 27.16 | 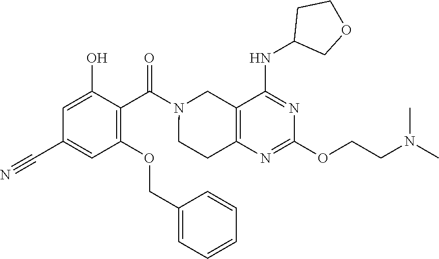 [SMILES: CN(C)CCOc1nc2c(c(NC3CCOC3)n1)CN(C(=O)c1c(O)cc(C#N)cc1OCc1ccccc1)CC2] | ¹H NMR (400 MHz, DMSO) δ 2.23- 1.96 (m, 7H), 4.61-3.55 (m, 10H), 5.20-4.85 (m, 2H), 7.42-6.55 (m, 8H). 6H obscured DMSO and water peaks. Exchangeable OH too broad to report. LCMS (LC-Method 1): 2.48 min, MS: ES+ 559.5 (M + 1). |
| | 3-(Benzyloxy)-4-(2-(2- | |
| | (dimethylamino)ethoxy)-4- | |
| | ((tetrahydrofuran-3-yl)amino)-5,6,7,8- | |
| | tetrahydropyrido[4,3-d]pyrimidine-6- | |
| | carbonyl)-5-hydroxybenzonitrile | |
| | Prepared from 2-(benzyloxy)-4-cyano-6- | |
| | hydroxybenzoic acid (Intermediate K) | |
| | and | |
| | 2-(2-(dimethylamino)ethoxy)-N- | |
| | (tetrahydrofuran-3-yl)-5,6,7,8- | |
| | tetrahydropyrido[4,3-d]pyrimidin-4- | |
| | amine dihydrochloride (Intermediate B8) | |
| | and purified using method prep-LC-5 | |
| 27.17 | 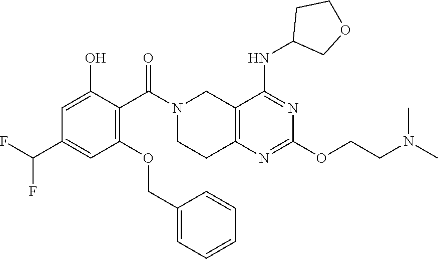 [SMILES: CN(C)CCOc1nc2c(c(NC3CCOC3)n1)CN(C(=O)c1c(O)cc(C(F)F)cc1OCc1ccccc1)CC2] | ¹H NMR (400 MHz, DMSO) δ 2.24- 1.95 (m, 7H), 4.60-3.55 (m, 10H), 5.17-4.84 (m, 2H), 7.35-6.67 (m, 9H). 6H observed to be under water and DMSO by HSQC. LCMS. Exchangeable OH too broad to report. (LC-Method 1): 2.73 min, MS: ES+ 584.4 (M + 1). |
| | (2-(Benzyloxy)-4-(difluoromethyl)-6- | |
| | hydroxyphenyl)(2-(2- | |
| | (dimethylamino)ethoxy)-4- | |
| | ((tetrahydrofuran-3-yl)amino)-7,8- | |
| | dihydropyrido[4,3-d]pyrimidin-6(5H)- | |
| | yl)methanone | |
| | Prepared from 2-(benzyloxy)-4- | |
| | (difluoromethyl)-6-hydroxybenzoic acid | |
| | (Intermediate D) and 2-(2- | |
| | (dimethylamino)ethoxy)-N- | |
| | (tetrahydrofuran-3-yl)-5,6,7,8- | |
| | tetrahydropyrido[4,3-d]pyrimidin-4- | |
| | amine dihydrochloride (Intermediate B8) | |
| | and purified using prep method prep-LC- | |
| | 12 | |
| 27.18 | 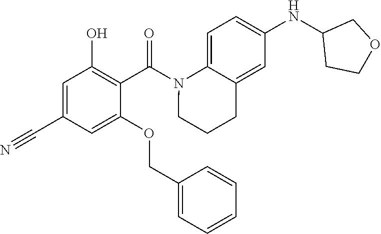 [SMILES: N#Cc1cc(O)c(C(=O)N2CCCc3cc(NC4CCOC4)ccc32)c(OCc2ccccc2)c1] | ¹H NMR (400 MHz, DMSO) δ 1.70- 1.63 (m, 3H), 2.09-1.95 (m, 1H), 2.44- 2.39 (m, 2H), 3.09-3.04 (m, 2H), 3.57- 3.39 (m, 3H), 3.85-3.78 (m, 1H), 5.20- 4.91 (m, 3H), 5.72 (s, 1H), 6.15-6.11 (m, 1H), 6.63-6.60 (m, 3H), 6.82 (s, 1H), 7.46-7.43 (m, 5H), 10.37 (s, 1H). LCMS (LC-Method 1): 4.32 min, MS: ES+ 470.4 (M + 1). |
| | 3-(Benzyloxy)-5-hydroxy-4-(6- | |
| | ((tetrahydrofuran-3-yl)amino)-1,2,3,4- | |
| | tetrahydroquinoline-1- | |
| | carbonyl)benzonitrile | |
| | Prepared from 2-(benzyloxy)-4-cyano-6- | |
| | hydroxybenzoic acid (Intermediate K) | |
| | and | |
| | N-(tetrahydrofuran-3-yl)-1,2,3,4- | |
| | tetrahydroquinolin-6-amine | |
| | dihydrochloride (Intermediate B1-4) and | |
| | purified using prep method prep-LC-13 | |
| 27.19 | 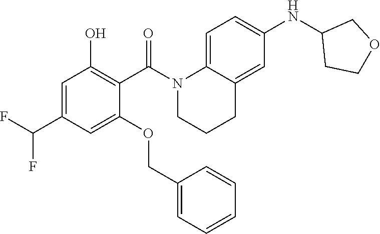 [SMILES: O=C(c1c(O)cc(C(F)F)cc1OCc1ccccc1)N1CCCc2cc(NC3CCOC3)ccc21] | ¹H NMR (400 MHz, DMSO) δ 1.89- 1.65 (m, 3H), 2.21-2.05 (m, 1H), 2.34- 2.26 (m, 1H), 2.72-2.58 (m, 1H), 3.52- 3.41 (m, 2H), 3.99-3.66 (m, 5H), 5.18- 4.82 (m, 2H), 5.65-5.57 (m, 1H), 6.56- 5.98 (m, 4H), 7.08-6.69 (m, 2H), 7.78- 7.22 (m, 5H), 10.08-10.05 (m, 1H). NMR is reported as a mixture of two rotamers. LCMS (LC-Method 2): 4.49 min, MS: ES+ 495.5 (M + 1). |
| | (2-(Benzyloxy)-4-(difluoromethyl)-6- | |
| | hydroxyphenyl)(6-((tetrahydrofuran-3- | |
| | yl)amino)-3,4-dihydroquinolin-1(2H)- | |
| | yl)methanone | |
| | Prepared from 2-(benzyloxy)-4- | |
| | (difluoromethyl)-6-hydroxybenzoic acid | |
| | (Intermediate D) and N- | |
| | (tetrahydrofuran-3-yl)-1,2,3,4- | |
| | tetrahydroquinolin-6-amine | |
| | dihydrochloride (Intermediate B1-4) and | |
| | purified using SFC method SFC-4 | |
| 27.20 | 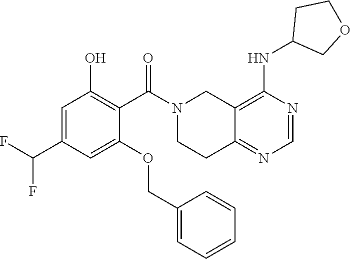 [SMILES: O=C(c1c(O)cc(C(F)F)cc1OCc1ccccc1)N1CCc2ncnc(NC3CCOC3)c2C1] | ¹H NMR (400 MHz, DMSO) δ 2.23- 1.74 (m, 2H), 2.71-2.59 (m, 2H), 4.13- 3.48 (m, 7H), 4.64-4.41 (m, 2H), 5.15- 4.87 (m, 2H), 7.30-6.53 (m, 9H), 8.32- 8.18 (m, 1H), 10.33-10.26 (m, 1H). NMR is reported as a mixture of rotamers. LCMS (LC-Method 1): 3.06 min, MS: ES+ 497.2 (M + 1) |
| | (2-(Benzyloxy)-4-(difluoromethyl)-6- | |
| | hydroxyphenyl)(4-((tetrahydrofuran-3- | |
| | yl)amino)-7,8-dihydropyrido[4,3- | |
| | d]pyrimidin-6(5H)-yl)methanone | |
| | Prepared from 2-(benzyloxy)-4- | |
| | (difluoromethyl)-6-hydroxybenzoic acid | |
| | (Intermediate D) and N- | |
| | (tetrahydrofuran-3-yl)-5,6,7,8- | |
| | tetrahydropyrido[4,3-d]pyrimidin-4- | |
| | amine dihydrochloride (Intermediate B7) | |
| | and purified using prep method prep-LC- | |
| | 2 | |
| 27.21 | 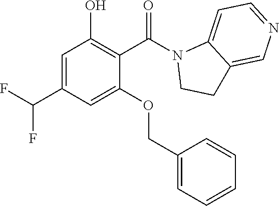 [SMILES: O=C(c1c(O)cc(C(F)F)cc1OCc1ccccc1)N1CCc2cnccc21] | ¹H NMR (400 MHz, DMSO) δ 3.16- 3.06 (m, 2H), 4.17-3.65 (m, 2H), 5.26- 4.95 (m, 2H), 7.39-6.70 (m, 8H), 8.46- 7.92 (m, 3H), 10.67-10.43 (m, 1H). LCMS (LC-Method 1): 3.32 min, MS: ES+ 397.3 (M + 1). |
| | (2-(Benzyloxy)-4-(difluoromethyl)-6- | |
| | hydroxyphenyl)(2,3-dihydro-1H- | |
| | pyrrolo[3,2-c]pyridin-1-yl)methanone | |
| | Prepared from 2-(benzyloxy)-4- | |
| | (difluoromethyl)-6-hydroxybenzoic acid | |
| | (Intermediate D) and 2,3-dihydro-1H- | |
| | pyrrolo[3,2-c]pyridin-5-ium chloride | |
| | (CAS 5912-19-6) and purified using prep | |
| | method prep-LC-3 | |
| 27.22 | 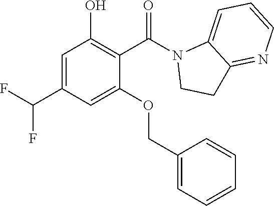 [SMILES: O=C(c1c(O)cc(C(F)F)cc1OCc1ccccc1)N1CCc2ncccc21] | ¹H NMR (400 MHz, DMSO) δ, 3.20- 3.06 (m, 2H), 4.18-3.66 (m, 2H), 5.24- 4.93 (m, 2H), 7.42-6.51 (m, 10H), 8.35-8.00 (m, 1H), 10.51-10.37 (m, 1H). LCMS (LC-Method 1): 4.05 min, MS: ES+ 397.3 (M + 1). |
| | (2-(Benzyloxy)-4-(difluoromethyl)-6- | |
| | hydroxyphenyl)(2,3-dihydro-1H- | |
| | pyrrolo[3,2-b]pyridin-1-yl)methanone | |
| | Prepared from 2-(benzyloxy)-4- | |
| | (difluoromethyl)-6-hydroxybenzoic acid | |
| | (Intermediate D) and 2,3-dihydro-1H- | |
| | pyrrolo[3,2-b]pyridin-4-ium chloride | |
| | (CAS 1443981-64-3) and | |
| | purified using prep Method prep-LC-3 | |
| 27.23 | 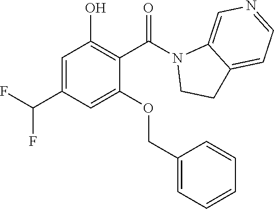 [SMILES: O=C(c1c(O)cc(C(F)F)cc1OCc1ccccc1)N1CCc2ccncc21] | ¹H NMR (400 MHz, DMSO) δ, 3.15- 2.98 (m, 1H), 4.13-3.62 (m, 3H), 5.17- 4.89 (m, 2H), 7.39-6.47 (m, 10H), 8.26-8.03 (m, 1H), 9.30-9.22 (m, 1H). LCMS (LC-Method 1): 3.35 min, MS: ES+ 397.3 (M + 1). |
| | (2-(Benzyloxy)-4-(difluoromethyl)-6- | |
| | hydroxyphenyl)(2,3-dihydro-1H- | |
| | pyrrolo[2,3-c]pyridin-1-yl)methanone | |
| | Prepared from 2-(benzyloxy)-4- | |
| | (difluoromethyl)-6-hydroxybenzoic acid | |
| | (Intermediate D) and 2,3-dihydro-1H- | |
| | pyrrolo[2,3-c]pyridine CAS 760919-39-9 | |
| | and purified using prep Method prep-LC- | |
| | 14 | |
| 27.24 | 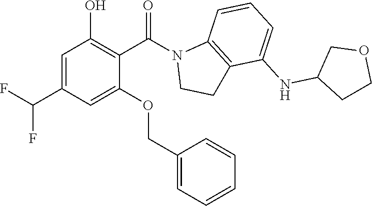 [SMILES: O=C(c1c(O)cc(C(F)F)cc1OCc1ccccc1)N1CCc2c(NC3CCOC3)cccc21] | ¹H NMR (400 MHz, DMSO) δ 1.87- 1.78 (m, 1H), 2.22-2.11 (m, 1H), 2.87- 2.80 (m, 2H), 4.05-3.51 (m, 7H), 5.19- 5.10 (m, 2H), 6.33-6.16 (m, 1H), 7.11- 6.65 (m, 5H), 7.50-7.25 (m, 6H), 10.32-10.17 (m, 1H). LCMS (LC-Method 1): 4.69 min, MS: ES+ 481.5 (M + 1). |
| | (2-(Benzyloxy)-4-(difluoromethyl)-6- | |
| | hydroxyphenyl)(4-((tetrahydrofuran-3- | |
| | yl)amino)indolin-1-yl)methanone | |
| | Prepared from 2-(benzyloxy)-4- | |
| | (difluoromethyl)-6-hydroxybenzoic acid | |
| | (Intermediate D) and N- | |
| | (Tetrahydrofuran-3-yl)indolin-4-amine | |
| | dihydrochloride (Intermediate B1-5) and | |
| | purified using prep-LC-9 | |

### Example 28: (S)-(4-(Difluoromethyl)-2-hydroxy-6-((1-methyl-1H-indazol-6-yl)methoxy)phenyl)(8-((tetrahydrofuran-3-yl)amino)-3,4-dihydroisoquinolin-2(1H)-yl)methanone

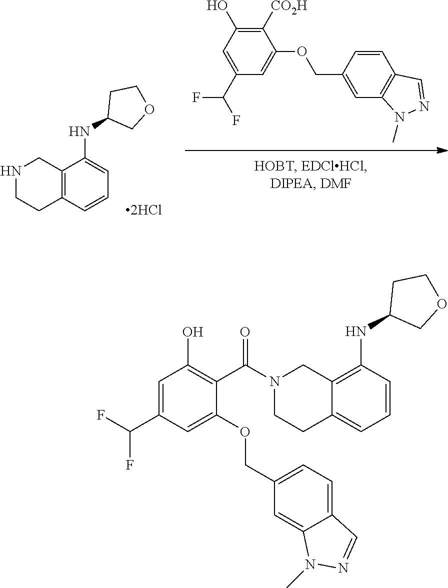 [SMILES: c1cc2c(c(N[C@H]3CCOC3)c1)CNCC2 ; Cn1ncc2ccc(COc3cc(C(F)F)cc(O)c3C(=O)O)cc21 ; Cn1ncc2ccc(COc3cc(C(F)F)cc(O)c3C(=O)N3CCc4cccc(N[C@H]5CCOC5)c4C3)cc21]

To a solution of 4-(difluoromethyl)-2-hydroxy-6-[(1-methylindazol-6-yl)methoxy]benzoic acid (Intermediate B11-1) (48 mg, 0.14 mmol, 1.0 eq), (S)—N-(tetrahydrofuran-3-yl)-1,2,3,4-tetrahydroisoquinolin-8-amine dihydrochloride (Intermediate B1-3) (40 mg, 0.14 mmol, 1.00 eq), EDC (27 mg, 0.14 mmol, 1.0 eq) and HOBT (21 mg, 0.14 mmol, 1.0 eq) in DMF (1.5 mL) was added DIPEA (0.12 mL, 0.69 mmol, 5.0 eq). The reaction mixture was stirred at room temperature for 16 hours then concentrated under vacuum. The residue was purified by prep HPLC (prep-LC-5) to give (S)-(4-(difluoromethyl)-2-hydroxy-6-((1-methyl-1H-indazol-6-yl)methoxy)phenyl)(8-((tetrahydrofuran-3-yl)amino)-3,4-dihydroisoquinolin-2(1H)-yl)methanone (22 mg, 28%).

¹H NMR (400 MHz, DMSO) d 1.94-1.85 (m, 1H), 2.22-2.16 (m, 1H), 2.71-2.66 (m, 2H), 3.44-3.39 (m, 2H), 3.65-3.60 (m, 1H), 3.79-3.71 (m, 1H), 3.82-3.82 (m, 3H), 3.96-3.84 (m, 2H), 4.04-4.04 (m, 1H), 4.60-4.58 (m, 2H), 4.98 (d, J=5.5 Hz, 1H), 5.29 (s, 2H), 6.50-6.37 (m, 2H), 6.74-6.72 (m, 1H), 6.88 (s, 1H), 7.01-6.94 (m, 2H), 7.12-7.08 (m, 1H), 7.56-7.54 (m, 1H), 7.69-7.65 (m, 1H), 7.98 (s, 1H), 10.20 (2, 1H). Rotameric mixture observed by NMR.

LCMS (Method 1): 4.62 mm MS: ES+ 549.5 (M+1).

The following compounds were prepared according to the method described above using the indicated intermediates.

| Ex. | Structure and Name | Analytical Data |
|---|---|---|
| 28.1 | 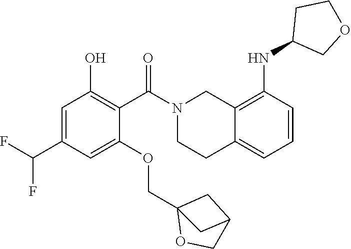 [SMILES: O=C(c1c(O)cc(C(F)F)cc1OCC12CC(CO1)C2)N1CCc2cccc(N[C@H]3CCOC3)c2C1] | ¹H NMR (400 MHz, DMSO) δ 1.29- 1.26 (m, 2H), 1.73-1.66 (m, 2H), 1.96-1.85 (m, 1H), 2.25-2.15 (m, 1H), 2.83-2.64 (m, 4H), 3.61- 3.60 (m, 3H), 3.88-3.72 (m, 2H), 4.13-3.91 (m, 3H), 4.22-4.20 (m, 2H), 4.67-4.43 (m, 2H), 4.98 (d, J = 5.5 Hz, 1H), 6.51-6.40 (m, 2H), 6.79-6.67 (m, 2H), 7.07-6.92 (m, 2H), 10.25-10.17 (m, 1H). Rotameric mixture observed by NMR. LCMS (Method 1): 4.50 min, MS: ES+ 501.6 (M + 1). |
| | (S)-(2-((2-Oxabicyclo[2.1.1]hexan-1- | |
| | yl)methoxy)-4-(difluoromethyl)-6- | |
| | hydroxyphenyl)(8-((tetrahydrofuran-3- | |
| | yl)amino)-3,4-dihydroisoquinolin-2(1H)- | |
| | yl)methanone | |
| | Prepared from 5-((2-oxabicyclo[2.1.1]hexan- | |
| | 1-yl)methoxy)-7-(difluoromethyl)-2,2- | |
| | dimethyl-4H-benzo[d][1,3]dioxin-4-one | |
| | (Intermediate B10-3) and (S)-N- | |
| | (tetrahydrofuran-3-yl)-1,2,3,4- | |
| | tetrahydroisoquinolin-8-amine dihydrochloride | |
| | (Intermediate B1-3) and purified using prep | |
| | method prep-LC-9 | |
| 28.2 | 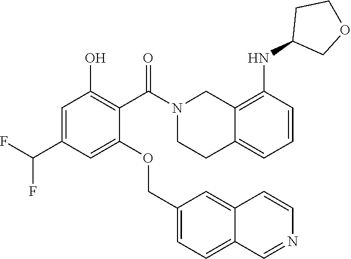 [SMILES: O=C(c1c(O)cc(C(F)F)cc1OCc1ccc2cnccc2c1)N1CCc2cccc(N[C@H]3CCOC3)c2C1] | ¹H NMR (400 MHz, DMSO) δ 1.93- 1.93 (m, 1H), 2.26-2.18 (m, 1H), 2.76-2.70 (m, 2H), 3.45-3.44 (m, 2H), 3.78-3.62 (m, 2H), 4.00- 3.83 (m, 2H), 4.07 (s, 1H), 4.71- 4.54 (m, 2H), 5.05-5.02 (m, 1H), 5.39-5.36 (m, 2H), 6.37 (d, J = 7.5 Hz, 1H), 6.52 (d, J = 7.9 Hz, 1H), 6.74 (s, 1H), 6.88-6.79 (m, 1H), 7.27-6.92 (m, 2H), 7.43 (d, J = 2.6 Hz, 1H), 7.64-7.59 (m, 1H), 7.81 (s, 1H), 8.07-8.03 (m, 1H), 8.44- 8.40 (m, 1H), 9.25 (s, 1H), 10.25 (s, 1H). Rotameric mixture observed by NMR. LCMS (Method 2) 3.43 min, MS: ES+ 546.5 (M + 1). |
| | (S)-(4-(Difluoromethyl)-2-hydroxy-6- | |
| | (isoquinolin-6-ylmethoxy)phenyl)(8- | |
| | ((tetrahydrofuran-3-yl)amino)-3,4- | |
| | dihydroisoquinolin-2(1H)-yl)methanone | |
| | Prepared from 4-(difluoromethyl)-2-hydroxy-6- | |
| | (isoquinolin-6-ylmethoxy)benzoic acid | |
| | (Intermediate B11-2) and (S)-N- | |
| | (tetrahydrofuran-3-yl)-1,2,3,4- | |
| | tetrahydroisoquinolin-8-amine dihydrochloride | |
| | (Intermediate B1-3) and purified using prep | |
| | method prep-LC-5 | |
| 28.3 | 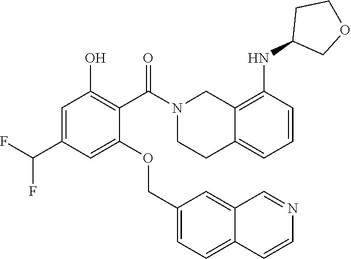 [SMILES: O=C(c1c(O)cc(C(F)F)cc1OCc1ccc2ccncc2c1)N1CCc2cccc(N[C@H]3CCOC3)c2C1] | ¹H NMR (400 MHz, DMSO) δ 2.07- 1.88 (m, 1H), 2.26-2.16 (m, 1H), 2.74-2.68 (m, 2H), 3.44-3.43 (m, 1H), 4.06-3.62 (m, 6H), 4.71- 4.53 (m, 2H), 5.04-5.00 (m, 1H), 5.36 (s, 2H), 6.39-6.35 (m, 1H), 6.52-6.48 (m, 1H), 6.73 (s, 1H), 6.91-6.81 (m, 1H), 7.00-6.94 (m, 2H), 7.79-7.69 (m, 2H), 7.97- 7.88 (m, 2H), 8.48-8.46 (m, 1H), 8.95 (d, J = 4.1 Hz, 1H), 10.26 (s, 1H). Rotameric mixture observed by NMR. LCMS (Method 2): 3.49 min, MS: ES+ 546.5 (M + 1). |
| | (S)-(4-(Difluoromethyl)-2-hydroxy-6- | |
| | (isoquinolin-7-ylmethoxy)phenyl)(8- | |
| | ((tetrahydrofuran-3-yl)amino)-3,4- | |
| | dihydroisoquinolin-2(1H)-yl)methanone | |
| | Prepared from 4-(difluoromethyl)-2-hydroxy-6- | |
| | (isoquinolin-7-ylmethoxy)benzoic acid | |
| | (Intermediate B11-4) and (S)-N- | |
| | (tetrahydrofuran-3-yl)-1,2,3,4- | |
| | tetrahydroisoquinolin-8-amine dihydrochloride | |
| | (Intermediate B1-3) and purified using prep | |
| | method prep-LC-12 | |
| 28.4 | 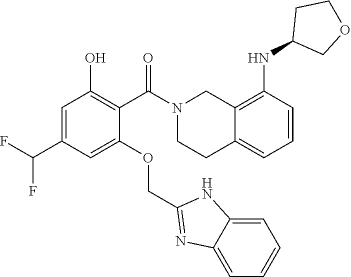 [SMILES: O=C(c1c(O)cc(C(F)F)cc1OCc1nc2ccccc2[nH]1)N1CCc2cccc(N[C@H]3CCOC3)c2C1] | ¹H NMR (400 MHz, DMSO) δ 1.92- 1.83 (m, 1H), 2.23-2.08 (m, 1H), 2.76-2.66 (m, 2H), 3.40-3.39 (m, 1H), 4.04-3.58 (m, 6H), 4.66- 4.40 (m, 2H), 4.93 (d, J = 4.6 Hz, 1H), 5.37-5.31 (m, 2H), 6.48- 6.38 (m, 2H), 6.81-6.65 (m, 2H), 7.03-6.93 (m, 2H), 7.21-7.13 (m, 2H), 7.48-7.44 (m, 1H), 7.60 (d, J = 7.0 Hz, 1H), 10.43-10.35 (m, 1H), 12.58-12.58 (m, 1H). LCMS (Method 2): 3.65 min, MS: ES+ 535.4 (M + 1). |
| | (S)-(2-((1H-Benzo[d]imidazol-2-yl)methoxy)- | |
| | 4-(difluoromethyl)-6-hydroxyphenyl)(8- | |
| | ((tetrahydrofuran-3-yl)amino)-3,4- | |
| | dihydroisoquinolin-2(1H)-yl)methanone | |
| | Prepared from 2-((1H-benzo[d]imidazol-2- | |
| | yl)methoxy)-4-(difluoromethyl)-6- | |
| | hydroxybenzoic acid (Intermediate B11-3) and | |
| | (S)-N-(tetrahydrofuran-3-yl)-1,2,3,4- | |
| | tetrahydroisoquinolin-8-amine dihydrochloride | |
| | (Intermediate B1-3) and purified using prep | |
| | method prep-LC-15 | |

### Example 29 1-(4-((2-(2-(Benzyloxy)-4-(difluoro methyl)-6-hydroxybenzoyl) isoindolin-5-yl) oxy) piperidin-1-yl) ethan-1-one

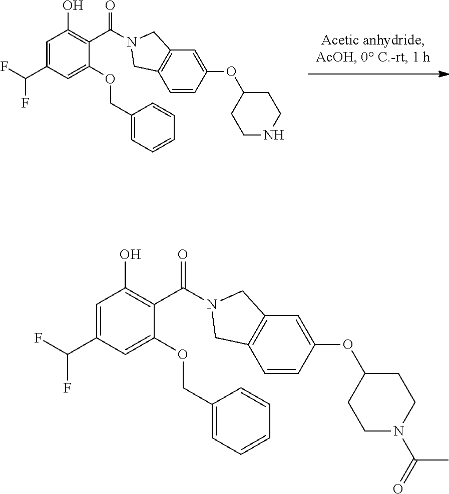 [SMILES: O=C(c1c(O)cc(C(F)F)cc1OCc1ccccc1)N1Cc2ccc(OC3CCNCC3)cc2C1 ; CC(=O)N1CCC(Oc2ccc3c(c2)CN(C(=O)c2c(O)cc(C(F)F)cc2OCc2ccccc2)C3)CC1]

To a stirred solution of (2-(benzyloxy)-4-(difluoromethyl)-6-hydroxy phenyl) (5-(piperidin-4-yloxy) isoindolin-2-yl) methanone (Example 26) (0.04 g, 0.081 mmol, 1. eq.) in acetic acid (0.4 mL) was added dropwise acetic anhydride (0.009 g, 0.088 mmol, 1.1 eq) at 0° C. and the reaction mixture was stirred at room temperature for 1 h. The resulting mixture was neutralized with saturated NaHCO₃ solution (pH~7-8), diluted with water (10 mL) and extracted with EtOAc (30 mL). The organic portion was dried over Na₂SO₄ and concentrated under vacuum. The crude material was purified by Prep TLC (3% MeOH in DCM) followed by pure fraction distillation yielding 1-(4-((2-(2-(benzyloxy)-4-(difluoro methyl)-6-hydroxybenzoyl) isoindolin-5-yl) oxy) piperidin-1-yl) ethan-1-one as an off white solid (0.029 g, Yield: 66.8%).

¹H NMR (DMSO-d6, 400 MHz): δ 1.48-1.60 (m, 2H), 1.87-2.03 (m, 5H), 3.10-3.30 (m, 2H), 3.63-3.67 (m, 1H), 3.83-3.86 (m, 1H), 4.38-4.45 (m, 2H), 4.49-4.67 (m, 3H), 5.20 (s, 2H), 6.75 (s, 1H), 6.85 (s, 1H), 6.88-6.96 (m, 2H), 7.05-7.10 (m, 1H), 7.16 (d, J=8 Hz, 1H), 7.26-7.35 (m, 5H).

LCMS (Method N): 1.547 min. MS: ES+ 537 (M+1).

HPLC (Method G): 6.486 min.

### Preparation of Intermediates

### Intermediate A (S)—N-(Tetrahydrofuran-3-yl)isoindolin-4-amine hydrochloride

### Step 1: tert-Butyl (S)-4-((tetrahydrofuran-3-yl)amino)isoindoline-2-carboxylate

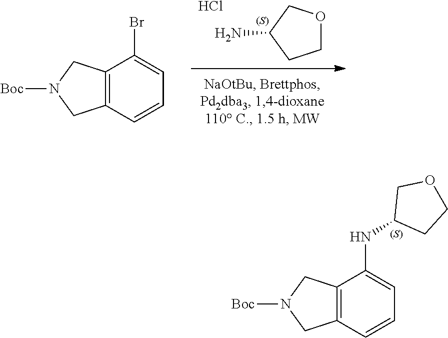

Performed in 2 parallel batches, each of 1 g scale: A mixture of tert-butyl 4-bromoisoindoline-2-carboxylate (Intermediate N step 1) (1.0 g, 3.36 mmol, 1 eq.), (S)-tetrahydrofuran-3-amine hydrochloride (CAS 204512-95-8) (0.54 g, 4.37 mmol, 1.3 eq.), NaOtBu (0.823 g, 8.4 mmol, 2.5 eq.) and Brettphos (0.108 g, 0.20 mmol, 0.06 eq.) in 1, 4-dioxane (10 mL) was degassed under N₂ gas for 30 mins. Pd₂(dba)₃ (0.092 g, 0.10 mmol, 0.03 eq.) was added to the reaction mixture at room temperature. The reaction mixture was heated to 110° C. using microwave radiation for 1.5 h. The resulting mixture was filtered through Celite® (filter material) and the filtrate was concentrated under vacuum. The crude material was purified twice by chromatography on silica (product eluted at 25% EtOAc in hexane) to yield the title compound. (0.650 g, Yield: 32.0%).

¹H NMR (DMSO-d6, 400 MHz): δ 1.42-1.47 (m, 9H), 1.83-1.84 (m, 1H), 2.14-2.16 (m, 1H), 3.56-3.59 (m, 1H), 3.71-3.73 (m, 1H), 3.82-3.84 (m, 1H), 3.89-3.93 (m, 1H), 4.01-4.04 (m, 1H), 4.41-4.52 (m, 4H), 5.36 (q, J=6.0, 12.0 Hz, 1H), 6.43-6.46 (m, 1H), 6.55 (t, J=7.6 Hz, 1H), 7.08 (t, J=7.6 Hz, 1H).

LCMS (Method A): 2.063 min, MS: ES+ 248.9 (M−56).

### Step 2: (S)—N-(Tetrahydrofuran-3-yl)isoindolin-4-amine hydrochloride

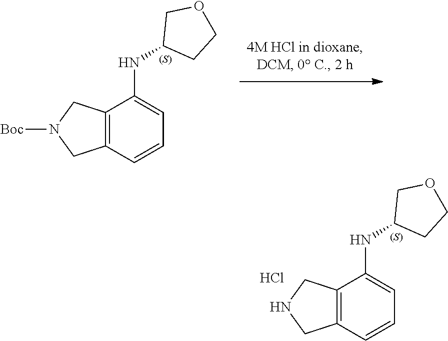

To a stirred solution of tert-butyl (S)-4-((tetrahydrofuran-3-yl)amino)isoindoline-2-carboxylate (step 1) (0.650 g, 2.13 mmol, 1.0 eq.) in DCM (6.5 mL), cooled to 0° C. was added dropwise 4M HCl in dioxane (6.5 mL) and the mixture was stirred at 0° C. for 2 h. The resulting mixture was concentrated under vacuum. The crude material was triturated with diethyl ether (5×10 mL) and material dried under high vacuum to yield the title compound (0.6 g, Yield: quantitative).

¹H NMR (DMSO-d6, 400 MHz): δ 1.81-1.85 (m, 1H), 2.17-2.22 (m, 1H), 3.56-3.59 (m, 1H), 3.69-3.74 (m, 1H), 3.81-3.92 (m, 2H), 4.03-4.06 (bs, 1H), 4.31-4.39 (m, 4H), 6.53-6.64 (m, 2H), 7.16 (t, J=7.6 Hz, 1H), 9.92 (s, 2H).

LCMS (Method A): 0.825 min, MS: ES+ 204.9 (M+1)

### Intermediate AA (S)—N-(Tetrahydrofuran-3-yl)-1,2,3,4-tetrahydroisoquinolin-8-amine hydrochloride

### Step 1: tert-Butyl (S)-8-((tetrahydrofuran-3-yl) amino)-3,4-dihydroisoquinoline-2(1H)-carboxylate

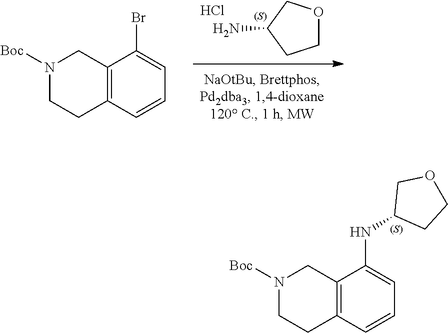

Performed in 2 parallel batches, each of 1 g scale: To a stirred solution of tert-butyl 8-bromo-3,4-dihydroisoquinoline-2(1H)-carboxylate (1.0 g, 3.20 mmol, 1 eq.) in 1,4-dioxane (10 mL) were added (S)-tetrahydrofuran-3-amine hydrochloride (0.33 g, 3.84 mmol, 1.2 eq) and NaOtBu (0.77 g, 8.01 mmol, 2.5 eq.) at room temperature. The reaction mixture was allowed to degas under N₂ gas at room temperature for 15 min. Brettphos (0.103 g, 0.19 mmol, 0.06 eq) and Pd₂(dba)₃ (0.117 g, 0.12 mmol, 0.04 eq) were added and the reaction mixture was heated to 120° C. and stirred for 1 h using microwave radiation. The resulting mixture was filtered through Celite® (filter material) and washed with 10% MeOH:DCM (300 mL). The organic layer was dried over Na₂SO₄, filtered, and concentrated under vacuum. The crude material was purified by chromatography on silica (product eluted at 20% EtOAc in hexane) yielding tert-butyl (S)-8-((tetrahydrofuran-3-yl) amino)-3,4-dihydroisoquinoline-2(1H)-carboxylate as a yellow solid (1.8 g, Yield: 92.4%).

¹H NMR (DMSO-d6, 400 MHz): δ 1.40 (s, 9H), 1.85-1.87 (m, 1H), 2.15-2.20 (m, 1H), 2.68 (t, J=5.6 Hz, 2H), 3.50 (s, 2H), 3.57-3.60 (m, 1H), 3.71-3.75 (m, 1H), 3.80-3.86 (m, 1H), 3.90-4.12 (m, 2H), 4.27 (s, 2H), 4.85 (s, 1H), 6.45 (d, J=8 Hz, 2H), 6.99 (t, J=7.6 Hz, 1H).

LCMS (Method A): 2.139 min, MS: ES+ 318.9 (M+1).

### Step 2: (S)—N-(Tetrahydrofuran-3-yl)-1,2,3,4-tetrahydroisoquinolin-8-amine hydrochloride

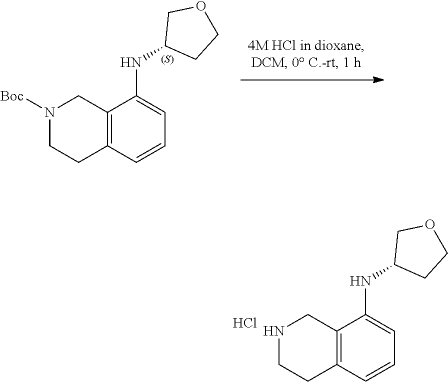

The title compound was prepared from tert-butyl (S)-8-((tetrahydrofuran-3-yl) amino)-3,4-dihydroisoquinoline-2(1H)-carboxylate (step 1) and 4M HCl in dioxane analogously to Intermediate A step 2.

¹H NMR (DMSO-d6, 400 MHz): δ 1.88-1.93 (m, 1H), 2.14-2.22 (m, 1H), 2.94 (t, J=6 Hz, 2H), 3.25 (t, J=4 Hz, 2H), 3.56-3.65 (m, 1H), 3.69-3.74 (m, 1H), 3.83-3.92 (m, 2H), 4.01-4.05 (m, 2H), 6.62 (t, J=5.2 Hz, 2H), 6.75-6.80 (m, 1H), 9.86 (s, 2H). H), 7.12 (t, J=7.6 Hz, 1H).

LCMS (Method A): 0.891 min, MS: ES+ 219.0 (M+1).

### Intermediate B (R)—N-(Tetrahydrofuran-3-yl)isoindolin-4-amine hydrochloride

### Step 1: tert-Butyl (R)-4-((tetrahydrofuran-3-yl)amino)isoindoline-2-carboxylate

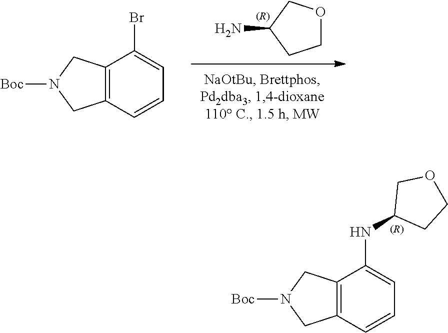

Performed in 3 parallel batches, each of 1 g scale: A mixture of tert-butyl 4-bromoisoindoline-2-carboxylate (Intermediate N step 1) (1.0 g, 3.36 mmol, 1 eq.), (R)-tetrahydrofuran-3-amine hydrochloride (CAS 111769-26-7) (0.38 g, 4.37 mmol, 1.3 eq.), NaOtBu (0.825 g, 8.42 mmol, 2.5 eq.) and Brettphos (0.108 g, 0.20 mmol, 0.06 eq.) in 1, 4-dioxane (10 mL) was degassed under N₂ gas for 30 mins. Pd₂(dba)₃ (0.092 g, 0.10 mmol, 0.03 eq.) was added and the reaction mixture was heated to 110° C. using microwave radiation for 1.5 h. The resulting mixture was filtered through Celite® (filter material) and the filtrate was concentrated under vacuum. The crude material was purified by chromatography on silica (product eluted at 15% EtOAc in hexane) to yield the title compound. (1.5 g, Yield: 28.0%).

¹H NMR (DMSO-d6, 400 MHz): δ 1.45-1.46 (m, 9H), 1.82-1.84 (m, 1H), 2.14-2.18 (m, 1H), 3.54-3.59 (m, 1H), 3.72-3.74 (m, 1H), 3.79-3.83 (m, 1H), 3.90 (t, J=8.4 Hz, 1H), 4.02-4.03 (m, 1H), 4.40-4.41 (m, 2H), 4.49-4.52 (m, 2H), 5.36-5.38 (m, 1H), 6.42-6.45 (m, 1H), 6.55 (t, J=7.6 Hz, 1H), 7.08 (t, J=7.6 Hz, 1H).

LCMS (Method A): 2.080 min, MS: ES+ 248.9 (M−56).

### Step 2: (R)—N-(Tetrahydrofuran-3-yl)isoindolin-4-amine hydrochloride

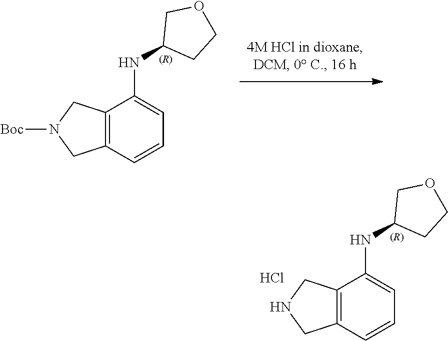

A stirred solution of tert-butyl (R)-4-((tetrahydrofuran-3-yl)amino)isoindoline-2-carboxylates (0.850 g, 2.79 mmol, 1.0 eq.) in DCM (9 mL) was cooled to 0° C. and treated dropwise with 4M HCl in dioxane (8.5 mL). The reaction mixture was allowed to warm to room temperature and stirred for 16 h. The resulting mixture was concentrated under vacuum. The crude material was triturated with n-pentane (7×10 mL) and dried under high vacuum to yield the title compound (0.580 g, Yield: quantitative).

¹H NMR (DMSO-d6, 400 MHz): δ 1.82-1.86 (m, 1H), 2.16-2.21 (m, 1H), 3.54-3.59 (m, 1H), 3.68-3.74 (m, 1H), 3.81-3.91 (m, 2H), 4.04 (bs, 1H), 4.33-4.41 (m, 4H), 6.53-6.65 (m, 1H), 6.94 (bs, 1H), 7.16 (t, J=8.0 Hz, 1H), 9.96 (s, 2H).

LCMS (Method A): 0.830 min, MS: ES+ 204.9 (M+1).

### Intermediate B1-1 N-(Tetrahydrofuran-3-yl)indolin-5-amine dihydrochloride

### Step 1: tert-Butyl 5-((tetrahydrofuran-3-yl)amino)indoline-1-carboxylate

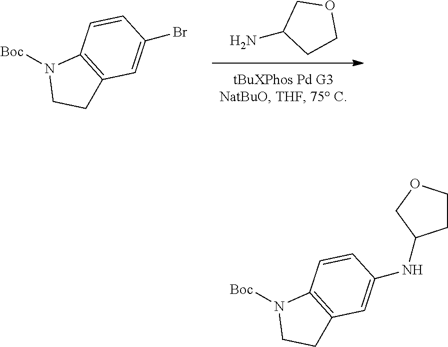 [SMILES: CC(C)(C)OC(=O)N1CCc2cc(Br)ccc21 ; NC1CCOC1 ; CC(C)(C)OC(=O)N1CCc2cc(NC3CCOC3)ccc21]

3-Aminotetrahydrofuran (0.11 mL, 1.3 mmol, 1.3 eq), sodium tert-butoxide (242 mg, 2.5 mmol, 2.5 eq) and 1-Boc-5-bromoindoline (300 mg, 1.0 mmol, 1.0 eq) were dissolved in anhydrous tetrahydrofuran (4.5 mL). The reaction was purged with nitrogen for 10 minutes and then t-BuXPhos Pd G3 (80 mg, 0.10 mmol, 0.1 eq) was added and the reaction was heated to 75° C. overnight. The reaction was quenched with water (40 mL) and extracted with EtOAc (3×50 mL). The combined organic extracts were washed with brine (40 mL) and filtered through phase separator paper. The combined organic extracts were concentrated under vacuum to give tert-butyl 5-((tetrahydrofuran-3-yl)amino)indoline-1-carboxylate (220 mg, 72%).

¹H NMR (400 MHz, DMSO) δ 1.48 (s, 9H), 1.75-1.67 (m, 1H), 2.18-2.08 (m, 1H), 2.95 (t, J=8.4 Hz, 2H), 3.47 (dd, J=3.5, 8.5 Hz, 1H), 3.93-3.67 (m, 6H), 5.49-5.44 (m, 1H), 6.35 (dd, J=2.3, 8.6 Hz, 1H), 6.47 (s, 1H), 7.47-7.41 (m, 1H).

MS: ES+ 305.1 (M+1).

### Step 2: N-(Tetrahydrofuran-3-yl)indolin-5-amine dihydrochloride

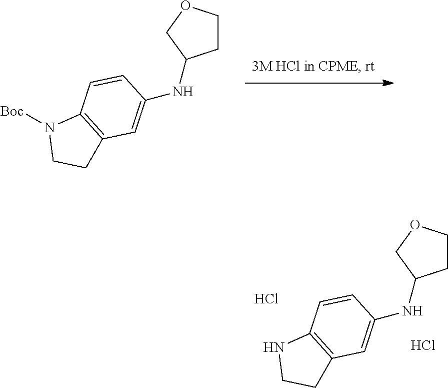 [SMILES: CC(C)(C)OC(=O)N1CCc2cc(NC3CCOC3)ccc21 ; Cl ; c1cc2c(cc1NC1CCOC1)CCN2 ; Cl]

3M HCl (2.0 mL, 6.0 mmol, 8.3 eq) in CPME was added to tert-butyl 5-((tetrahydrofuran-3-yl)amino)indoline-1-carboxylate (step 1) (220 mg, 0.72 mmol, 1.0 eq) in MeOH (0.5 mL) and DCM (0.5 mL). The reaction mixture was stirred at room temperature for 20 h and then the solvent was removed in vacuo to give N-(tetrahydrofuran-3-yl)indolin-5-amine dihydrochloride (200 mg). The compound was directly used in the next steps without further purification.

¹H NMR (400 MHz, MeOD) δ 1.95-1.86 (m, 1H), 2.28-2.18 (m, 1H), 3.27-3.22 (m, 2H), 3.79-3.70 (m, 5H), 3.98-3.91 (m, 1H), 4.17-4.11 (m, 1H), 7.04-6.99 (m, 1H), 7.09 (s, 1H), 7.37-7.34 (m, 1H).

MS: ES+ 205 (M+1).

The compounds of the following tabulated Intermediates were prepared analogously to Intermediate B1-1 steps 1 and 2 from the indicated intermediates.

| Int. | Structure and Name | Analytical Data |
|---|---|---|
| B1-2 | 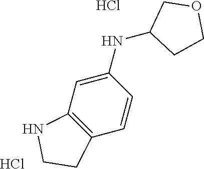 [SMILES: Cl ; c1cc2c(cc1NC1CCOC1)NCC2 ; Cl] | Used directly in next step without further purification MS: ES+ 205.1 (M + 1) |
| | N-(Tetrahydrofuran-3-yl)indolin-6-amine | |
| | dihydrochloride. | |
| | Prepared from tert-butyl 6-bromoindoline-1- | |
| | carboxylate and 3-aminotetrahydrofuran | |
| B1-3 | 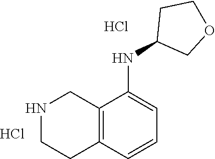 [SMILES: Cl ; c1cc2c(c(N[C@H]3CCOC3)c1)CNCC2 ; Cl] | ¹H NMR (400 MHz, DMSO) δ 1.89- 1.80 (m, 1H), 2.24-2.14 (m, 1H), 2.95-2.90 (m, 2H), 3.30-3.23 (m, 2H), 3.59 (dd, J = 3.9, 8.8 Hz, 1H), 4.06-3.69 (m, 6H), 6.55-6.51 (m, 2H), 7.09 (t, J = 7.8 Hz, 1H), 9.58 (s, 2H). MS: ES+ 219 (M + 1). |
| | (S)-N-(Tetrahydrofuran-3-yl)-1,2,3,4- | |
| | tetrahydroisoquinolin-8-amine | |
| | dihydrochloride. | |
| | Prepared from tert-butyl 8-bromo-3,4- | |
| | dihydroisoquinoline-2(1H)-carboxylate and | |
| | (S)-3-aminotetrahydrofuran tosylate | |
| B1-4 | 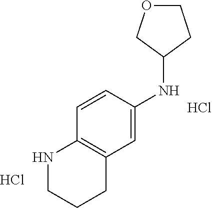 [SMILES: Cl ; c1cc2c(cc1NC1CCOC1)CCCN2 ; Cl] | Used directly in next step without further purification. MS: ES+ 308.2 (M + 1) |
| | N-(Tetrahydrofuran-3-yl)-1,2,3,4- | |
| | tetrahydroquinolin-6-amine dihydrochloride. | |
| | Prepared from tert-butyl 6-bromo-3,4- | |
| | dihydroquinoline-1(2H)-carboxylate and 3- | |
| | aminotetrahydrofuran | |
| B1-5 | 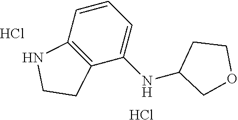 [SMILES: Cl ; c1cc2c(c(NC3CCOC3)c1)CCN2 ; Cl] | ¹H NMR (400 MHz, MeOD) δ 1.61- 1.55 (m, 2H), 1.79-1.72 (m, 2H), 2.03-1.92 (m, 1H), 2.38-2.29 (m, 1H), 3.16 (t, J = 7.3 Hz, 2H), 3.74 (dd, J = 3.3, 9.0 Hz, 1H), 3.89-3.82 (m, 3H), 4.01-3.96 (m, 2H), 4.26-4.18 (m, 1H), 6.76 (dd, J = 8.0, 13.4 Hz, 2H), 7.29 (t, J = 8.0 Hz, 1H). MS: ES+ 308.2 (M + 1). |
| | N-(Tetrahydrofuran-3-yl)indolin-4-amine | |
| | dihydrochloride. | |
| | Prepared from tert-butyl 4-bromoindoline-1- | |
| | carboxylate and 3-aminotetrahydrofuran | |

### Intermediate B2-1 6-(4-Methyl piperazin-1-yl)-2,3-dihydro-1H-pyrrolo[3,4-c]pyridine dihydrobromide

### Step 1: 4-Methyl-N,N-di(prop-2-yn-1-yl)benzenesulfonamide

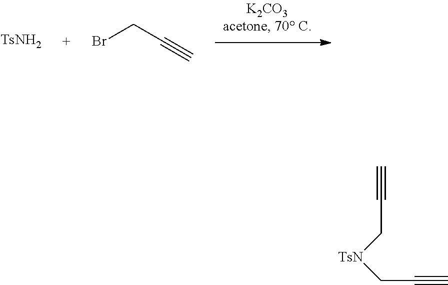

Propargyl bromide [80% (wt.) in toluene, 1.6 mL, 14 mmol, 2.5 eq] was added dropwise to a suspension of p-toluenesulfonamide (1.0 g, 5.8 mmol, 1.0 eq) and K₂CO₃ (2.4 g, 17.5 mmol, 3.0 eq) in acetone (10 mL). The mixture was heated to 70° C. for 6 h. The resulting mixture was cooled to room temperature, diluted with EtOAc (50 mL) then washed with water (30 mL) and brine (20 mL), dried over MgSO₄ and concentrated under vacuum. The residue was purified by chromatography on silica, eluting with a gradient of 50-100% EtOAc in cyclohexane to give 4-methyl-N,N-di(prop-2-yn-1-yl)benzenesulfonamide (1.0 g, 71%)) as a low melting point solid.

¹H NMR (400 MHz, DMSO) δ 2.41-2.39 (m, 3H), 3.24-3.23 (m, 2H), 4.10-4.08 (m, 4H), 7.44-7.40 (m, 2H), 7.75-7.71 (m, 2H).

MS: ES+ 248.0 (M+1).

### Step 2: 6-(4-Methylpiperazin-1-yl)-2-tosyl-2,3-dihydro-1H-pyrrolo[3,4-c]pyridine

Pentamethylcyclopentadienyltris (acetonitrile)ruthenium(II) hexafluorophosphate (21 mg, 0.04 mmol, 0.05 eq) and 4-methylpiperazine-1-carbonitrile (202 mg, 1.6 mmol, 2.0 eq) were dissolved in DCM (4.5 mL) and added to 4-methyl-N,N-di(prop-2-yn-1-yl)benzenesulfonamide (step 1) (200 mg, 0.81 mmol, 1.0 eq) in a degassed solution of DCM (0.5 mL). The reaction mixture was stirred at room temperature for 3 h and then evaporated to dryness to give a fine grey solid. The residue was purified by chromatography on silica, eluting with cyclohexane/EtOAc followed by 0-5% MeOH in DCM to yield 6-(4-methylpiperazin-1-yl)-2-tosyl-2,3-dihydro-1H-pyrrolo[3,4-c]pyridine (216 mg, 72%) as a white solid.

¹H NMR (400 MHz, DMSO) δ 2.19-2.18 (m, 3H), 2.38-2.32 (m, 7H), 3.43-3.36 (m, 4H), 4.45 (s, 4H), 6.71-6.69 (m, 1H), 7.44-7.40 (m, 2H), 7.76-7.72 (m, 2H), 7.98 (s, 1H). MS: ES+ 373.1 (M+1).

### Step 3: 6-(4-Methylpiperazin-1-yl)-2,3-dihydro-1H-pyrrolo[3,4-c]pyridine dihydrobromide

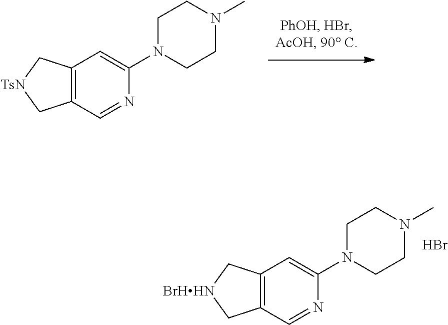

A mixture of phenol (327 mg, 3.5 mmol, 6.0 eq), 6-(4-methylpiperazin-1-yl)-2-tosyl-2,3-dihydro-1H-pyrrolo[3,4-c]pyridine (step 2) (216 mg, 0.58 mmol, 1.0 eq) and HBr (33%, 0.63 mL, 3.5 mmol, 6.0 eq) in acetic acid (1.50 mL) was heated to 90° C. for 6 h. The resulting mixture was allowed to cool to room temperature and stand overnight. The mixture was concentrated under vacuum and the residue was suspended in EtOAc (20 mL). The resulting solid was isolated by filtration and dried under high vacuum to yield 6-(4-methylpiperazin-1-yl)-2,3-dihydro-1H-pyrrolo[3,4-c]pyridine dihydrobromide (267 mg, quant.) as a pink powder.

¹H NMR (400 MHz, DMSO) δ 2.87-2.83 (m, 3H), 3.23-3.04 (m, 4H), 3.57-3.52 (m, 2H), 4.52-4.35 (m, 6H), 7.07-7.05 (m, 1H), 8.21 (s, 1H), 9.50-9.49 (m, 2H), 9.87 (s, 1H).

MS: ES+ 219.1 (M+1).

The following tabulated Intermediates were prepared analogously to Intermediate B2-1 steps 1, 2 and 3 from the indicated intermediates.

| Intermediate. | Structure | Data |
|---|---|---|
| B2-2 | 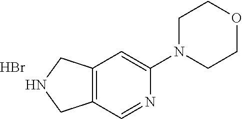 [SMILES: Br ; c1nc(N2CCOCC2)cc2c1CNC2] | ¹H NMR (400 MHz, DMSO) δ 3.56-3.50 (m, 4H), 3.75-3.71 (m, 4H), 4.54-4.43 (m, 4H), 7.15 (s, 1H), 8.17 (s, 1H), 9.51-9.49 (m, 2H). MS: ES+ 360.0 (M + 1). |
| | 4-(2,3-Dihydro-1H-pyrrolo[3,4-c]pyridin- | |
| | 6-yl)morpholine hydrobromide | |
| | Prepared from 4-methyl-N,N-di(prop-2- | |
| | yn-1-yl)benzenesulfonamide and | |
| | morpholine-4-carbonitrile | |

### Intermediate B3 1-(2,3-Dihydro-1H-pyrrolo[3,4-c]pyridin-6-yl)-N,N-dimethylmethanamine dihydrobromide

### Step 1: 4-Methyl-N,N-di(prop-2-yn-1-yl)benzenesulfonamide

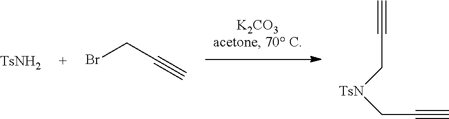

Propargyl bromide (80% (wt.) in toluene, 1.6 mL, 14.6 mmol, 2.5 eq] was added dropwise to a suspension of p-toluenesulfonamide (1.00 g, 5.8 mmol, 1.0 eq) and K₂CO₃ (2.4 g, 17.5 mmol, 3.0 eq) in acetone (10 mL). The mixture was heated to 70° C. for 6 h. The resulting mixture was cooled to room temperature, diluted with EtOAc (50 mL) then washed with water (30 mL) and brine (20 mL), dried over MgSO₄ and concentrated under vacuum. The residue was purified by chromatography on silica, eluting with a gradient of 50-100% EtOAc in cyclohexane to give 4-methyl-N,N-di(prop-2-yn-1-yl)benzenesulfonamide (1.03 g, 71%) as a low melting point solid.

¹H NMR (400 MHz, DMSO) δ 2.41-2.39 (m, 3H), 3.24-3.23 (m, 2H), 4.10-4.08 (m, 4H), 7.44-7.40 (m, 2H), 7.75-7.71 (m, 2H). MS: ES+ 248.0 (M+1).

### Step 2: 6-(Chloromethyl)-2-tosyl-2,3-dihydro-1H-pyrrolo[3,4-c]pyridine

A solution of 4-methyl-N,N-di(prop-2-yn-1-yl)benzenesulfonamide (step 1) (500 mg, 2.0 mmol, 1.0 eq) in DCM (20 mL) was added dropwise to a solution of chloroacetonitrile (0.19 mL, 3.0 mmol, 1.5 eq) and chloro(pentamethylcyclopentadienyl)(cyclooctadiene)ruthenium(II) (16 mg, 0.04 mmol, 0.02 eq) in DCM (5 mL) under nitrogen via syringe pump over 2 hours. The reaction mixture was stirred overnight and then concentrated under vacuum to ca. 5 mL. Purification of the crude material by chromatography on silica eluting with 1:1 EtOAc:cyclohexane yielded 6-(chloromethyl)-2-tosyl-2,3-dihydro-1H-pyrrolo[3,4-c]pyridine (605 mg, 93%) as an off-white solid.

¹H NMR (400 MHz, DMSO) δ 2.37-2.36 (m, 3H), 4.63-4.61 (m, 4H), 4.74 (s, 2H), 7.46-7.40 (m, 3H), 7.78-7.74 (m, 2H), 8.44 (s, 1H).

MS: ES+ 323.0, 324.9 (M+1).

### Step 3: N,N-Dimethyl-1-(2-tosyl-2,3-dihydro-1H-pyrrolo[3,4-c]pyridin-6-yl)methanamine

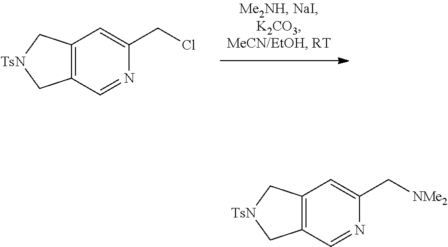 [SMILES: Cc1ccc(S(=O)(=O)N2Cc3cnc(CCl)cc3C2)cc1 ; Cc1ccc(S(=O)(=O)N2Cc3cnc(CN(C)C)cc3C2)cc1]

5.6M Dimethylamine (0.28 mL, 1.6 mmol, 2.5 eq) was added to a mixture of 6-(chloromethyl)-2-tosyl-2,3-dihydro-1H-pyrrolo[3,4-c]pyridine (step 2) (200 mg, 0.62 mmol, 1.0 eq), sodium iodide (9.3 mg, 0.06 mmol, 0.1 eq) and K₂CO₃ (428 mg, 3.1 mmol, 5.0 eq) in acetonitrile (2.0 mL). The mixture was stirred for 10 min then further acetonitrile (4.0 mL) was added to aid stirring of the thick suspension. The reaction mixture was stirred at room temperature overnight. The resulting mixture was filtered through Celite®, washed with EtOAc and the combined organics were concentrated under vacuum to yield N,N-dimethyl-1-(2-tosyl-2,3-dihydro-1H-pyrrolo[3,4-c]pyridin-6-yl)methanamine (220 mg, 100%) as an off-white solid.

¹H NMR (400 MHz, DMSO) δ 2.14 (s, 6H), 2.35 (s, 3H), 3.47 (s, 2H), 4.0-4.58 (m, 4H), 7.32 (s, 1H), 7.2-7.39 (m, 2H), 7.7-7.73 (m, 2H), 8.35 (s, 1H).

MS: ES+ 332.0 (M+1).

### Step 4: 1-(2,3-Dihydro-1H-pyrrolo[3,4-c]pyridin-6-yl)-N,N-dimethylmethanamine dihydrobromide

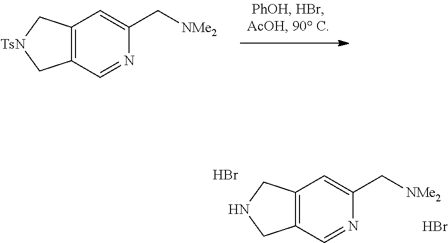 [SMILES: Cc1ccc(S(=O)(=O)N2Cc3cnc(CN(C)C)cc3C2)cc1 ; Br ; CN(C)Cc1cc2c(cn1)CNC2 ; Br]

A mixture of phenol (375 mg, 4.0 mmol, 6.0 eq), N,N-dimethyl-1-(2-tosyl-2,3-dihydro-1H-pyrrolo[3,4-c]pyridin-6-yl)methanamine (step 3) (220 mg, 0.6 mmol, 1.0 eq) and HBr (33%, 0.72 mL, 4.0 mmol, 6.0 eq) in acetic acid (1.5 mL) was heated to 90° C. overnight. After cooling to room temperature, the resulting mixture was concentrated under vacuum and the residue was suspended in EtOAc (20 mL). The solid was isolated by filtration and dried under high vacuum to yield 1-(2,3-dihydro-1H-pyrrolo[3,4-c]pyridin-6-yl)-N,N-dimethylmethanamine dihydrobromide (279 mg, quant.) as a beige powder.

¹H NMR (400 MHz, DMSO) δ 2.84-2.80 (m, 6H), 4.55-4.50 (m, 2H), 4.66-4.60 (m, 4H), 7.61-7.58 (m, 1H), 8.76-8.73 (m, 1H), 9.59 (s, 2H), 9.96 (s, 1H).

MS: ES+ 178.1 (M+1).

### Intermediate B4 7-((1-Methylpiperidin-4-yl)oxy)-2,3,4,5-tetrahydro-1H-benzo[d]azepine ditrifluoroacetate

### Step 1: tert-Butyl 7-((1-methylpiperidin-4-yl)oxy)-1,2,4,5-tetrahydro-3H-benzo[d]azepine-3-carboxylate

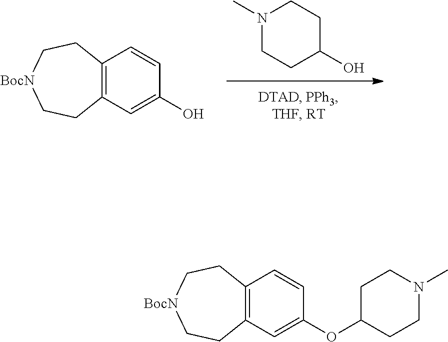

N-Methyl-4-piperidinol (0.27 mL, 2.3 mmol, 1.2 eq) was added to a stirred solution of tert-butyl 7-hydroxy-1,2,4,5-tetrahydro-3-benzazepine-3-carboxylate (500 mg, 1.9 mmol, 1.0 eq), triphenylphosphine (598 mg, 2.3 mmol, 1.2 eq) and di-tert-butyl azodicarboxylate (525 mg, 2.3 mmol, 1.2 eq) in tetrahydrofuran (15 mL) under a nitrogen atmosphere. The mixture was stirred at room temperature overnight. The resulting mixture was diluted with sodium hydrogen carbonate (sat. aq., 100 mL) and EtOAc (100 mL). The mixture was separated and the organic layer was washed with brine (sat. aq., 100 mL), dried over sodium sulfate, filtered and concentrated under vacuum. The crude material was loaded onto a 20 g Isolute® SCX cartridge then eluted with 10% MeOH in DCM followed by 20% (7M NH₃ in MeOH) in DCM. The product fractions were concentrated under vacuum and further purified by chromatography on silica eluting with a gradient of 0-6% (7M NH₃ in MeOH) in DCM to give tert-butyl 7-((1-methylpiperidin-4-yl)oxy)-1,2,4,5-tetrahydro-3H-benzo[d]azepine-3-carboxylate (206 mg, 27%) as a clear oil.

¹H NMR (400 MHz, CDCl₃) δ 1.49 (s, 9H), 1.88-1.76 (m, 2H), 2.04-1.94 (m, 2H), 2.30-2.29 (m, 5H), 2.74-2.64 (m, 2H), 2.82-2.78 (m, 4H), 3.57-3.48 (m, 4H), 4.31-4.22 (m, 1H), 6.70-6.63 (m, 2H), 7.02-6.97 (m, 1H).

MS: ES+ 361.2 (M+1).

### Step 2: 7-((1-Methylpiperidin-4-yl)oxy)-2,3,4,5-tetrahydro-1H-benzo[d]azepine ditrifluoroacetate

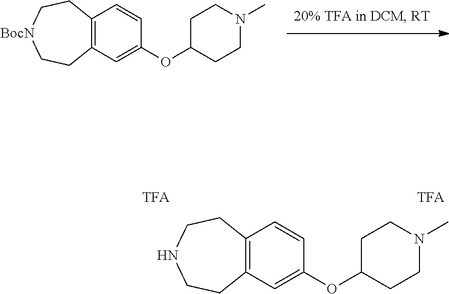 [SMILES: CN1CCC(Oc2ccc3c(c2)CCN(C(=O)OC(C)(C)C)CC3)CC1 ; CN1CCC(Oc2ccc3c(c2)CCNCC3)CC1]

Trifluoroacetic acid (0.52 mL, 6.7 mmol, 24 eq) was added to a solution of tert-butyl 7-((1-methylpiperidin-4-yl)oxy)-1,2,4,5-tetrahydro-3H-benzo[d]azepine-3-carboxylate (step 1) (103 mg, 0.29 mmol, 1.0 eq) in DCM (2.5 mL). The mixture was stirred for 2.5 h then concentrated under vacuum to give 7-((1-methylpiperidin-4-yl)oxy)-2,3,4,5-tetrahydro-1H-benzo[d]azepine ditrifluoroacetate (140 mg, quant.) which was used directly in the next step without further purification.

MS: ES+ 261.2 (M+1).

### Intermediate B5 2-(Indolin-4-yloxy)-N,N-dimethylethan-1-amine dihydrochloride

### Step 1: tert-Butyl 4-hydroxyindoline-1-carboxylate

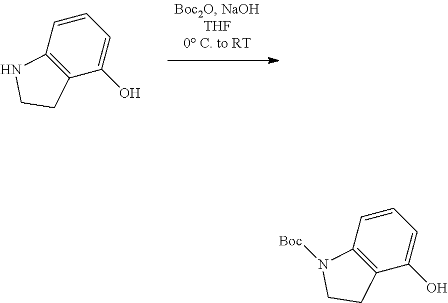 [SMILES: Oc1cccc2c1CCN2 ; CC(C)(C)OC(=O)N1CCc2c(O)cccc21]

Indolin-4-ol (50 mg, 0.37 mmol, 1.0 eq) was dissolved in THF (2.0 mL) and treated with 2M sodium hydroxide (0.43 mL, 0.85 mmol, 2.3 eq). The reaction mixture was cooled to 0° C., Di-tert-butyl dicarbonate (0.09 mL, 0.39 mmol, 1.05 eq) was added and the mixture was stirred overnight at room temperature. The resulting mixture was diluted with water (20 mL) and the phase were separated. The aqueous phase was extracted with EtOAc (3×30 mL) and the combined organic phases were filtered through phase separator paper and concentrated under vacuum to give tert-butyl 4-hydroxyindoline-1-carboxylate (45 mg, 51%).

¹H NMR (400 MHz, DMSO) δ 1.48 (t, J=8.1 Hz, 9H), 2.89 (t, J=8.0 Hz, 2H), 3.87 (t, J=8.4 Hz, 2H), 6.42-6.36 (m, 1H), 6.93 (t, J=7.5 Hz, 1H), 7.14 (broad s, 1H), 9.36 (s, 1H).

MS: ES+ 236.2 (M+1).

### Step 2: tert-Butyl 4-(2-(dimethylamino)ethoxy)indoline-1-carboxylate

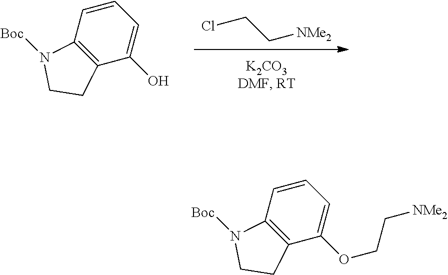 [SMILES: CC(C)(C)OC(=O)N1CCc2c(O)cccc21 ; CN(C)CCCl ; CN(C)CCOc1cccc2c1CCN2C(=O)OC(C)(C)C]

A solution of tert-butyl 4-hydroxyindoline-1-carboxylate (step 1) (97 mg, 0.41 mmol, 1.0 eq) in N,N-dimethylformamide (1.8 mL) was treated with potassium carbonate (114 mg, 0.83 mmol, 2.0 eq) and the mixture was stirred at room temperature for 10 mins. 2-Chloro-N,N-dimethylethylamine hydrochloride (65 mg, 0.45 mmol, 1.1 eq) was added and the reaction mixture was stirred at room temperature overnight. An additional portion of K₂CO₃ (85 mg, 0.62 mmol, 1.5 eq) was added and stirring continued overnight. The resulting mixture was diluted in EtOAc (10 mL) and washed with sat NaHCO₃ (10 mL). The organic phase was filtered through a phase separator paper and concentrated under vacuum. The crude material was dissolved in MeOH and passed through an Isolute® SCX cartridge. The cartridge was washed with MeOH then the product was released with 3M NH₃ in MeOH. The product fractions were concentrated under vacuum to give tert-butyl 4-(2-(dimethylamino)ethoxy)indoline-1-carboxylate (118 mg, 75%)

¹H NMR (400 MHz, DMSO) δ 1.51-1.47 (m, 9H), 2.23-2.22 (m, 6H), 2.62 (t, J=5.8 Hz, 2H), 2.92 (t, J=8.7 Hz, 2H), 3.90 (t, J=8.7 Hz, 2H), 4.07 (t, J=5.8 Hz, 2H), 6.63-6.59 (m, 1H), 7.13-7.07 (m, 1H). Aromatic CH not observed due to restricted rotation.

MS: ES+ 307.2 (M+1).

### Step 3: 2-(Indolin-4-yloxy)-N,N-dimethylethan-1-amine dihydrochloride

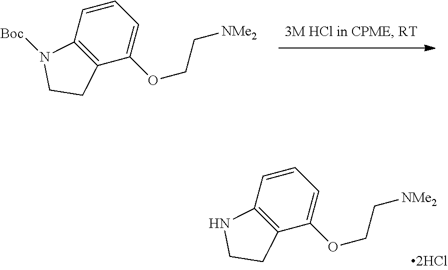 [SMILES: CN(C)CCOc1cccc2c1CCN2C(=O)OC(C)(C)C ; CN(C)CCOc1cccc2c1CCN2]

tert-Butyl 4-[2-(dimethylamino)ethoxy]indoline-1-carboxylate (80%, 108 mg, 0.28 mmol, 1.0 eq) in methyl alcohol (0.50 mL) was treated with 3M HCl in CPME (0.85 mL, 2.5 mmol, 9.0 eq) and the reaction mixture was stirred at room temperature overnight. The solvent was removed under reduced pressure to give 2-(indolin-4-yloxy)-N,N-dimethylethan-1-amine dihydrochloride (78.7 mg, quant.). The product was used directly in the next step without further purification.

MS: ES+ 207.2 (M+1).

### Intermediate B6 2,3-Dihydro-1H-pyrrolo[3,4-c]pyridin-6-amine dihydrochloride

### Step 1: tert-Butyl 6-chloro-1,3-dihydro-2H-pyrrolo[3,4-c]pyridine-2-carboxylate

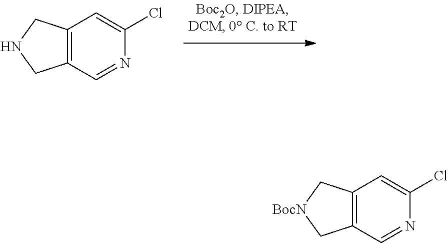 [SMILES: Clc1cc2c(cn1)CNC2 ; CC(C)(C)OC(=O)N1Cc2cnc(Cl)cc2C1]

DIPEA (0.68 mL, 3.9 mmol, 3.0 eq) was added dropwise to a cooled (0° C.) solution of 6-chloro-2,3-dihydro-1H-pyrrolo[3,4-c]pyridine hydrochloride (250 mg, 1.3 mmol, 1.0 eq) and di-tert-butyl dicarbonate (0.39 mL, 1.7 mmol, 1.3 eq) in DCM (5.0 mL). The mixture was stirred for 10 minutes at 0° C. then stirred at room temperature for 3 h. The resulting mixture was diluted with DCM (10 mL) and washed with water (10 mL). The organics were dried over MgSO₄ and concentrated under vacuum. The crude residue was purified by chromatography on silica eluting with 0-50% EtOAc in cyclohexane to give tert-butyl 6-chloro-1,3-dihydro-2H-pyrrolo[3,4-c]pyridine-2-carboxylate (201 mg, 60%) as a white solid.

¹H NMR (400 MHz, DMSO) δ 1.46 (s, 9H), 4.63-4.58 (m, 4H), 7.55-7.52 (m, 1H), 8.40-8.37 (m, 1H).

MS: ES+ 255.0, 257.0, (M+1).

### Step 2: tert-Butyl 6-((2,4-dimethoxybenzyl)amino)-1,3-dihydro-2H-pyrrolo[3,4-c]pyridine-2-carboxylate

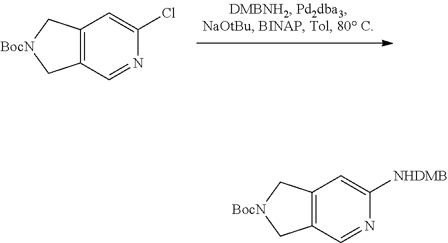

(±)-2,2-Bis(diphenylphosphino)-1,1-binaphthalene (123 mg, 0.2 mmol, 0.25 eq), tris(dibenzylideneacetone)dipalladium(0) (94 mg, 0.10 mmol, 0.13 eq), sodium tert-butoxide (243 mg, 2.53 mmol, 3.2 eq) and tert-butyl 6-chloro-1,3-dihydro-2H-pyrrolo[3,4-c]pyridine-2-carboxylate (step 1) (201 mg, 0.79 mmol, 1.0 eq) were dissolved in degassed toluene (15 mL). 2,4-Dimethoxybenzylamine (0.43 mL, 2.8 mmol, 3.6 eq) was added and the reaction mixture was heated to 80° C. overnight. The resulting mixture was allowed to cool to room temperature and diluted with water (60 mL). The aqueous phase was extracted with EtOAc (3×50 mL) and the combined organic phases were washed with brine, dried over MgSO₄, filtered and concentrated under vacuum. The residue was purified by chromatography on silica eluting with a gradient of 0-100% EtOAc in cyclohexane. The residue was dissolved in Et₂O, concentrated under vacuum and dried to give tert-butyl 6-((2,4-dimethoxybenzyl)amino)-1,3-dihydro-2H-pyrrolo[3,4-c]pyridine-2-carboxylate (257 mg, 84%) as a beige foam.

¹H NMR (400 MHz, CDCl₃) δ 1.50 (s, 9H), 3.79 (s, 3H), 3.83 (s, 3H), 4.38 (dd, J=2.8, 5.8 Hz, 2H), 4.61-4.48 (m, 4H), 4.97-4.91 (m, 1H), 6.32-6.24 (m, 1H), 6.48-6.40 (m, 2H), 7.21-7.18 (m, 1H), 8.03-7.96 (m, 1H).

MS: ES+ 386.1 (M+1).

### Step 3: 2,3-Dihydro-1H-pyrrolo[3,4-c]pyridin-6-amine

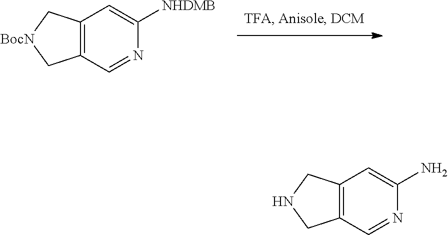

Anisole (0.07 mL, 0.64 mmol, 1.0 eq) was added to a solution of tert-butyl 6-((2,4-dimethoxybenzyl)amino)-1,3-dihydro-2H-pyrrolo[3,4-c]pyridine-2-carboxylate (step 2) (246 mg, 0.64 mmol, 1.0 eq) in trifluoroacetic acid (6.0 mL) and heated under an inert atmosphere to 70° C. for 4 h. The resulting mixture was allowed to cool to room temperature and the solvents were removed under a stream of nitrogen. The residue was applied to an Isolute® SCX column and eluted with DCM, 50% MeOH in DCM then 50% (7M NH₃ in MeOH) in DCM. The basic fraction was concentrated under vacuum to give 2,3-dihydro-1H-pyrrolo[3,4-c]pyridin-6-amine (86.3 mg, quant.) was isolated as a pale brown solid. The compound was used directly in the next step without further purification.

¹H NMR (400 MHz, DMSO) b 3.91 (s, 4H), 4.03-4.01 (m, 1H), 5.65 (s, 2H), 6.33 (s, 1H), 7.79 (s, 1H).

MS: ES+ 135 (M+1).

### Intermediate B7 N-(Tetrahydrofuran-3-yl)-5,6,7,8-tetrahydropyrido[4,3-d]pyrimidin-4-amine dihydrochloride

### Step 1: tert-Butyl 4-((tetrahydrofuran-3-yl)amino)-7,8-dihydropyrido[4,3-d]pyrimidine-6(5H)-carboxylate hydrochloride

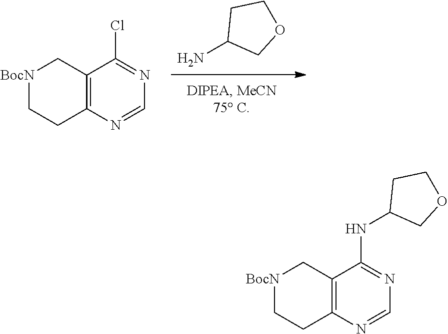 [SMILES: CC(C)(C)OC(=O)N1CCc2ncnc(Cl)c2C1 ; NC1CCOC1 ; CC(C)(C)OC(=O)N1CCc2ncnc(NC3CCOC3)c2C1]

To a solution of tert-butyl 4-chloro-7,8-dihydro-5H-pyrido[4,3-d]pyrimidine-6-carboxylate (150 mg, 0.56 mmol, 1.0 eq) in acetonitrile (2.00 mL) was added DIPEA (0.22 mL, 1.3 mmol, 2.3 eq) and 3-aminotetrahydrofuran (0.059 mL, 0.69 mmol, 1.2 eq). The reaction mixture was stirred at room temperature for 24 h. Additional 3-aminotetrahydrofuran (0.07 mL, 0.83 mmol, 1.5 eq) and DIPEA (0.19 mL, 1.1 mmol, 2.0 eq) were added and the reaction was heated to 75° C. for a further 24 h. Another portion of 3-aminotetrahydrofuran (0.06 mL, 0.67 mmol, 1.2 eq) and DIPEA (0.15 mL, 0.83 mmol, 1.5 eq) were added and stirring continued at 75° C. for a further 6 h. Water (20 mL) was added and the aqueous phase was extracted with EtOAc (3×30 mL). The combined organic phases were filtered through phase separator paper and concentrated under vacuum. The crude residue was purified by chromatography on silica eluting with 0 to 100% EtOAc in cyclohexane followed by 0 to 5% MeOH in DCM to yield tert-butyl 4-((tetrahydrofuran-3-yl)amino)-7,8-dihydropyrido[4,3-d]pyrimidine-6(5H)-carboxylate (84 mg, 47%).

¹H NMR (400 MHz, DMSO) δ 1.44-1.43 (m, 9H), 2.01-1.92 (m, 1H), 2.21-2.09 (m, 1H), 2.62 (t, J=5.8 Hz, 2H), 3.62-3.55 (m, 3H), 3.75-3.68 (m, 1H), 3.92-3.83 (m, 2H), 4.24 (s, 2H), 4.63-4.55 (m, 1H), 6.85 (d, J=3.6 Hz, 1H), 8.29 (s, 1H).

MS: ES+ 321.1 (M+1).

### Step 2: N-(Tetrahydrofuran-3-yl)-5,6,7,8-tetrahydropyrido[4,3-d]pyrimidin-4-amine hydrochloride

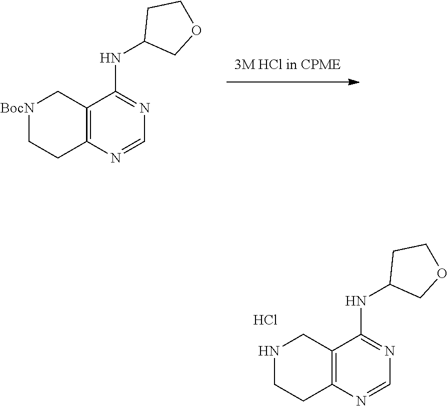 [SMILES: CC(C)(C)OC(=O)N1CCc2ncnc(NC3CCOC3)c2C1 ; Cl ; c1nc2c(c(NC3CCOC3)n1)CNCC2]

tert-Butyl 4-((tetrahydrofuran-3-yl)amino)-7,8-dihydropyrido[4,3-d]pyrimidine-6(5H)-carboxylate (84 mg, 0.26 mmol, 1.0 eq) was dissolved in methyl alcohol (0.50 mL) and 3M HCl in CPME (1.2 mL, 3.5 mmol, 13 eq) was added. The reaction mixture was stirred overnight at room temperature. The solvent was removed under reduced pressure to give N-(tetrahydrofuran-3-yl)-5,6,7,8-tetrahydropyrido[4,3-d]pyrimidin-4-amine hydrochloride (67.5 mg, quant.).

MS: ES+ 221.1 (M+1)

### Intermediate B8 2-(2-(Dimethylamino)ethoxy)-N-(tetrahydrofuran-3-yl)-5,6,7,8-tetrahydropyrido[4,3-d]pyrimidin-4-amine dihydrochloride

### Step 1: tert-Butyl 2-chloro-4-((tetrahydrofuran-3-yl)amino)-7,8-dihydropyrido[4,3-d]pyrimidine-6(5H)-carboxylate

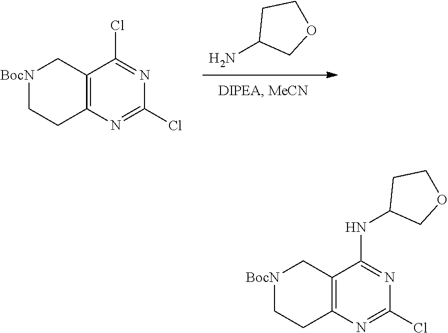 [SMILES: CC(C)(C)OC(=O)N1CCc2nc(Cl)nc(Cl)c2C1 ; NC1CCOC1 ; CC(C)(C)OC(=O)N1CCc2nc(Cl)nc(NC3CCOC3)c2C1]

Tert-butyl 2,4-dichloro-7,8-dihydro-5H-pyrido[4,3-d]pyrimidine-6-carboxylate (150 mg, 0.49 mmol, 1.0 eq) was dissolved in acetonitrile (2.0 mL) and DIPEA (0.19 mL, 1.1 mmol, 2.3 eq) and 3-aminotetrahydrofuran (0.05 mL, 0.61 mmol, 1.2 eq) were added. The reaction mixture was left stirring for 24 h at room temperature after which time water (20 mL) was added. The aqueous phase was extracted with EtOAc (3×30 mL) and the combined organic phases were filtered through phase separator paper and concentrated under vacuum to yield tert-butyl 2-chloro-4-((tetrahydrofuran-3-yl)amino)-7,8-dihydropyrido[4,3-d]pyrimidine-6(5H)-carboxylate (128 mg, 73%).

MS: ES+ 355.0, 358.0 (M+1).

### Step 2: tert-Butyl 2-(2-(dimethylamino)ethoxy)-4-((tetrahydrofuran-3-yl)amino)-7,8-dihydropyrido[4,3-d]pyrimidine-6(5H)-carboxylate

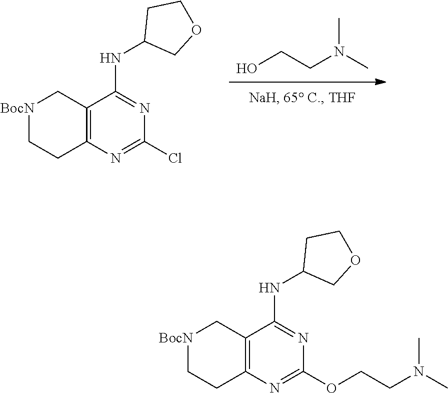

tert-Butyl 2-chloro-4-((tetrahydrofuran-3-yl)amino)-7,8-dihydropyrido[4,3-d]pyrimidine-6(5H)-carboxylate (step 1) (128 mg, 0.36 mmol, 1.0 eq) and 2-dimethylaminoethanol (0.18 mL, 1.8 mmol, 5.0 eq) were dissolved in tetrahydrofuran (10 mL) and treated slowly with sodium hydride (26 mg, 1.1 mmol, 3.0 eq). The reaction was heated at 65° C. for 6 h. Additional 2-dimethylaminoethanol (0.22 mL, 2.2 mmol, 6.0 eq) was added, followed by slow addition of sodium hydride (43 mg, 1.8 mmol, 5.0 eq). The reaction mixture was heated at 65° C. for 5 h. The reaction was quenched with water (25 mL) followed by NaHCO₃ (10 mL). The aqueous phase was extracted with EtOAc (3×40 mL) and the combined organic phases were washed with brine (30 mL), filtered through phase separator paper and dried under vacuum. The crude material was dissolved in MeOH and DCM and purified using an Isolute® SCX cartridge, first washing the cartridge with MeOH and then eluting the product with 7M NH₃:MeOH in DCM. The product fractions were concentrated under vacuum and further purified by chromatography on silica eluting with 0 to 100% [10% (7M NH₃:MeOH) in DCM]:DCM to give tert-butyl 2-(2-(dimethylamino)ethoxy)-4-((tetrahydrofuran-3-yl)amino)-7,8-dihydropyrido[4,3-d]pyrimidine-6(5H)-carboxylate (107 mg, 73%).

¹H NMR (400 MHz, DMSO) δ 1.44 (s, 9H), 2.18 (s, 6H), 2.56-2.52 (m, 2H), 3.19-3.16 (m, 4H), 3.62-3.53 (m, 4H), 3.94-3.82 (m, 2H), 4.28-4.17 (m, 5H), 6.88 (s, 1H). NMR contains rotamers.

MS: ES+ 408 (M+1).

### Step 3: 2-(2-(Dimethylamino)ethoxy)-N-(tetrahydrofuran-3-yl)-5,6,7,8-tetrahydropyrido[4,3-d]pyrimidin-4-amine dihydrochloride

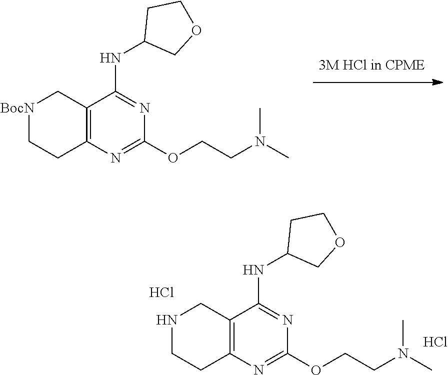 [SMILES: CN(C)CCOc1nc2c(c(NC3CCOC3)n1)CN(C(=O)OC(C)(C)C)CC2 ; Cl ; CN(C)CCOc1nc2c(c(NC3CCOC3)n1)CNCC2 ; Cl]

tert-Butyl 2-(2-(dimethylamino)ethoxy)-4-((tetrahydrofuran-3-yl)amino)-7,8-dihydropyrido[4,3-d]pyrimidine-6(5H)-carboxylate (step 2) (103 mg, 0.25 mmol, 1.0 eq) was dissolved in methyl alcohol (1.0 mL) and treated with 3M HCl in CPME (1.2 mL, 3.5 mmol, 14.0 eq). The reaction mixture was stirred at room temperature for 3 h. The solvent was removed under vacuum to give 2-(2-(dimethylamino)ethoxy)-N-(tetrahydrofuran-3-yl)-5,6,7,8-tetrahydropyrido[4,3-d]pyrimidin-4-amine dihydrochloride (96.2 mg, quant.).

MS: ES+ 308.2 (M+1).

### Intermediate B9-1 7-(Difluoromethyl)-2,2-dimethyl-5-[(1-methylindazol-6-yl)methoxy]-1,3-benzodioxin-4-one

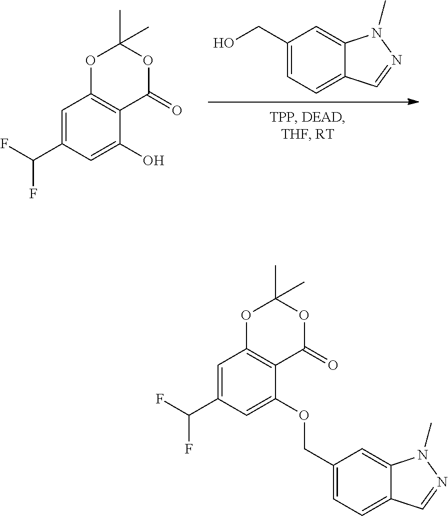 [SMILES: CC1(C)OC(=O)c2c(O)cc(C(F)F)cc2O1 ; Cn1ncc2ccc(CO)cc21 ; Cn1ncc2ccc(COc3cc(C(F)F)cc4c3C(=O)OC(C)(C)O4)cc21]

To a stirred solution of (1-methylindazol-6-yl)methanol (135 mg, 0.8 mmol, 1.0 eq), 7-(difluoromethyl)-5-hydroxy-2,2-dimethyl-4H-benzo[d][1,3]dioxin-4-one (Intermediate D step 8) (200 mg, 0.8 mmol, 1.0 eq) and triphenylphosphine (322 mg, 1.2 mmol, 1.5 eq) in dry tetrahydrofuran (2.0 mL) was added dropwise diethyl azodicarboxylate (0.19 mL, 1.2 mmol, 1.5 eq). The reaction mixture was stirred at room temperature for 16 hours then concentrated under vacuum. The residue was purified by chromatography on silica eluting with a gradient of 0-50% EtOAc in cyclohexane to afford 7-(difluoromethyl)-2,2-dimethyl-5-[(1-methylindazol-6-yl)methoxy]-1,3-benzodioxin-4-one (326 mg, 100%) as a white solid.

¹H NMR (400 MHz, CDCl₃) δ 1.76 (s, 6H), 4.12-4.12 (m, 3H), 5.42 (s, 2H), 6.58 (t, J=56.1 Hz, 1H), 6.75 (s, 1H), 6.86 (s, 1H), 7.20-7.16 (m, 1H), 7.74-7.71 (m, 1H), 7.97 (s, 2H).

MS: ES+ 389.1 (M+1).

The following compound was prepared according to the method described above using the indicated intermediates.

| Int. | Structure and Name | Data |
|---|---|---|
| B9-2 | 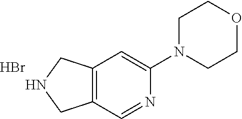 [SMILES: Br ; c1nc(N2CCOCC2)cc2c1CNC2] | ¹H NMR (400 MHz, DMSO) δ 1.70 (s, 6H), 5.55 (s, 2H), 7.18- 6.90 (m, 3H), 7.86-7.80 (m, 2H), 8.20-8.15 (m, 2H), 8.53 (d, J = 5.8 Hz, 1H), 9.33 (s, 1H). MS: ES+ 386.1 (M + 1). |
| | 7-(Difluoromethyl)-5-(isoquinolin-6-ylmethoxy)-2,2- | |
| | dimethyl-4H-benzo[d][1,3]dioxin-4-one | |
| | Prepared from 7-(difluoromethyl)-5-hydroxy-2,2- | |
| | dimethyl-4H-benzo[d][1,3]dioxin-4-one | |
| | (Intermediate D step 8) and 6-isoquinolinemethanol | |

### Intermediate B10-1 5-((1H-Benzo[d]imidazol-2-yl)methoxy)-7-(difluoromethyl)-2,2-dimethyl-4H-benzo[d][1,3]dioxin-4-one

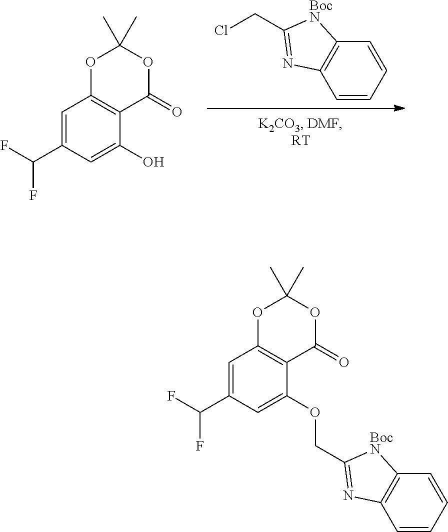 [SMILES: CC1(C)OC(=O)c2c(O)cc(C(F)F)cc2O1 ; CC(C)(C)OC(=O)n1c(CCl)nc2ccccc21 ; CC(C)(C)OC(=O)n1c(COc2cc(C(F)F)cc3c2C(=O)OC(C)(C)O3)nc2ccccc21]

To a stirred solution of 7-(difluoromethyl)-5-hydroxy-2,2-dimethyl-4H-benzo[d][1,3]dioxin-4-one (Intermediate D step 8) (148 mg, 0.6 mmol, 1.0 eq) in DMF (4.5 mL) was added potassium carbonate (95 mg, 0.7 mmol, 1.1 eq). The reaction was stirred for 15 minutes then tert-butyl 2-(chloromethyl)benzimidazole-1-carboxylate (183 mg, 0.7 mmol, 1.1 eq) was added dropwise at 0° C. The reaction mixture was allowed to stir at room temperature for 16 hours then partitioned between EtOAc (30 mL) and water (30 mL). The organic phase was separated, washed with brine, dried over magnesium sulfate, filtered and concentrated under vacuum. The residue was purified by chromatography on silica eluting with a gradient of 0-25% EtOAc in cyclohexane to afford tert-butyl 2-[[7-(difluoromethyl)-2,2-dimethyl-4-oxo-1,3-benzodioxin-5-yl]oxymethyl]benzimidazole-1-carboxylate (230 mg, 80%) as a white solid.

¹H NMR (400 MHz, CDCl₃) δ 1.71-1.70 (m, 15H), 5.70 (s, 2H), 6.57 (t, J=55.7 Hz, 1H), 6.76 (s, 1H), 7.00 (s, 1H), 7.41-7.33 (m, 2H), 7.76 (dd, J=1.6, 7.0 Hz, 1H), 7.98 (dd, J=1.5, 7.3 Hz, 1H).

MS: ES+ 475.0 (M+1).

The following compounds were prepared according to the method described above using the indicated intermediates.

| Intermediate | Structure and Name | Data |
|---|---|---|
| B10-2 | 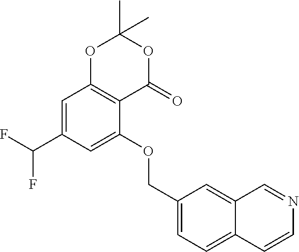 [SMILES: CC1(C)OC(=O)c2c(OCc3ccc4ccncc4c3)cc(C(F)F)cc2O1] | ¹H NMR (400 MHz, CDCl₃) δ 1.58 (s, 6H), 5.45 (s, 2H), 6.58 (t, J = 55.9 Hz, 1H), 6.76 (s, 1H), 6.86 (s, 1H), 7.67 (d, J = 5.8 Hz, 1H), 7.90-7.88 (m, 2H), 8.25 (s, 1H), 8.55 (d, J = 5.8 Hz, 1H), 9.31 (s, 1H). MS: ES+ 386.1 (M + 1). |
| | 7-(Difluoromethyl)-5-(isoquinolin-7- | |
| | ylmethoxy)-2,2-dimethyl-4H- | |
| | benzo[d][1,3]dioxin-4-one | |
| | Prepared from 7-(difluoromethyl)-5- | |
| | hydroxy-2,2-dimethyl-4H- | |
| | benzo[d][1,3]dioxin-4-one (Intermediate | |
| | D step 8) and 7- | |
| | (bromomethyl)isoquinoline | |
| | hydrobromide | |
| B10-3 | 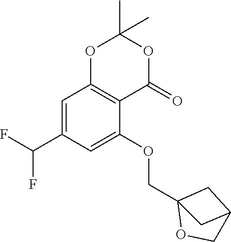 [SMILES: CC1(C)OC(=O)c2c(OCC34CC(CO3)C4)cc(C(F)F)cc2O1] | ¹H NMR (400 MHz, CDCl₃) δ 1.61-1.58 (m, 2H), 1.72 (s, 6H), 2.10-2.04 (m, 2H), 2.98 (t, J = 3.3 Hz, 1H), 3.86 (s, 2H), 4.35 (s, 2H), 6.71-6.41 (m, 2H), 6.76 (s, 1H). MS: ES+ 341.1 (M + 1). |
| | 5-((2-Oxabicyclo[2.1.1]hexan-1- | |
| | yl)methoxy)-7-(difluoromethyl)-2,2- | |
| | dimethyl-4H-benzo[d][1,3]dioxin-4-one | |
| | Prepared from 7-(difluoromethyl)-5- | |
| | hydroxy-2,2-dimethyl-4H- | |
| | benzo[d][1,3]dioxin-4-one (Intermediate | |
| | D step 8) and 1-(iodomethyl)-2- | |
| | oxabicyclo[2.1.1]hexane | |

### Intermediate B11-1 4-(Difluoromethyl)-2-hydroxy-6-[(1-methylindazol-6-yl)methoxy]benzoic acid

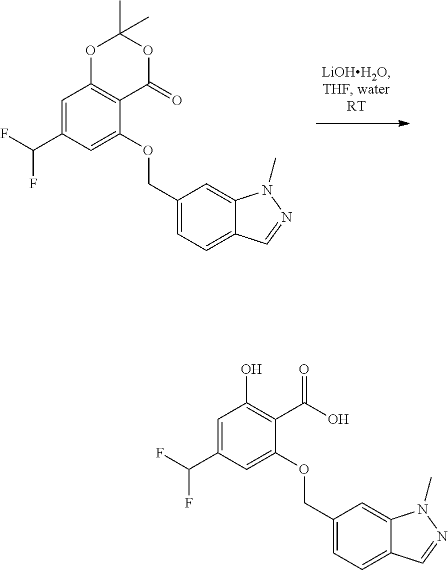 [SMILES: Cn1ncc2ccc(COc3cc(C(F)F)cc4c3C(=O)OC(C)(C)O4)cc21 ; Cn1ncc2ccc(COc3cc(C(F)F)cc(O)c3C(=O)O)cc21]

Lithium hydroxide monohydrate (106 mg, 2.5 mmol, 3.0 eq) was added in one portion to a solution of 7-(difluoromethyl)-2,2-dimethyl-5-[(1-methyl indazol-6-yl)methoxy]-1,3-benzodioxin-4-one (Intermediate B9-1) (326 mg, 0.8 mmol, 1.0 eq) in tetrahydrofuran (3.5 mL) and water (1.8 mL). The mixture was stirred at room temperature for 16 hours. The mixture was cooled and diluted with water (10 mL) then acidified with 1M KHSO₄ (2 mL). The aqueous layer was extracted with DCM (3×10 mL). The combined organic extracts were dried over MgSO₄ and concentrated under vacuum to afford 4-(difluoromethyl)-2-hydroxy-6-[(1-methylindazol-6-yl)methoxy]benzoic acid (191 mg, 65%) as a white solid.

¹H NMR (400 MHz, CDCl₃) δ 4.11 (s, 3H), 5.45 (s, 2H), 6.58 (t, J=55.6 Hz, 1H), 6.79 (s, 1H), 6.88 (s, 1H), 7.23-7.20 (m, 1H), 7.49-7.48 (m, 1H), 7.84-7.82 (m, 1H), 8.04-8.03 (m, 1H), 11.36-11.32 (m, 1H), 12.32 (s, 1H).

MS: ES+ 349.1 (M+1).

The following compounds were prepared according to the method described above using the indicated intermediates.

| Intermediate | Structure and Name | Data |
|---|---|---|
| B11-2 | 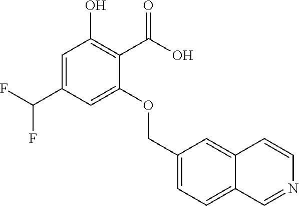 [SMILES: O=C(O)c1c(O)cc(C(F)F)cc1OCc1ccc2cnccc2c1] | ¹H NMR (400 MHz, DMSO) δ 5.39 (s, 2H), 6.69 (s, 1H), 6.81 (s, 1H), 6.92 (t, J = 54.5 Hz, 1H), 7.81-7.71 (m, 2H), 8.02 (s, 1H), 8.18-8.13 (m, 1H), 8.52 (d, J = 5.6 Hz, 1H), 9.32 (s, 1H), 11.90 (s, 1H). 1 exchangeable proton not visible. MS: ES+ 346.1 (M + 1). |
| | 4-(Difluoromethyl)-2-hydroxy-6- | |
| | (isoquinolin-6-ylmethoxy)benzoic acid | |
| | Prepared from 7-(Difluoromethyl)-5- | |
| | (isoquinolin-6-ylmethoxy)-2,2-dimethyl-4H- | |
| | benzo[d][1,3]dioxin-4-one (Intermediate B9-2) | |
| B11-3 | 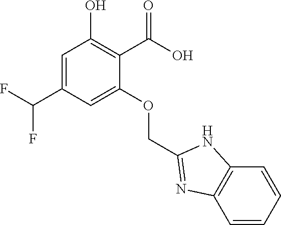 [SMILES: O=C(O)c1c(O)cc(C(F)F)cc1OCc1nc2ccccc2[nH]1] | ¹H NMR (400 MHz, DMSO) δ 5.40-5.39 (m, 2H), 6.71 (s, 1H), 7.07-6.78 (m, 2H), 7.24- 7.20 (m, 2H), 7.59-7.56 (m, 2H). exchangeable protons not visible. MS: ES+ 334.9 (M + 1). |
| | 2-((1H-Benzo[d]imidazol-2-yl)methoxy)-4- | |
| | (difluoromethyl)-6-hydroxybenzoic acid | |
| | Prepared from 5-((1H-Benzo[d]imidazol-2- | |
| | yl)methoxy)-7-(difluoromethyl)-2,2- | |
| | dimethyl-4H-benzo[d][1,3]dioxin-4-one | |
| | (Intermediate B10-1) | |
| B11-4 | 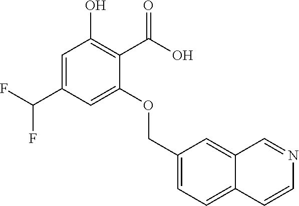 [SMILES: O=C(O)c1c(O)cc(C(F)F)cc1OCc1ccc2ccncc2c1] | ¹H NMR (400 MHz, DMSO) δ 5.37 (s, 2H), 6.69 (s, 1H), 7.07- 6.78 (m, 2H), 7.86-7.82 (m, 2H), 8.01 (d, J = 8.5 Hz, 1H), 8.18 (s, 1H), 8.52 (d, J = 5.8 Hz, 1H), 9.29 (s, 1H), 11.93 (s, 2H). MS: ES+ 349.0 (M + 1). |
| | 4-(Difluoromethyl)-2-hydroxy-6- | |
| | (isoquinolin-7-ylmethoxy)benzoic acid | |
| | Prepared from 7-(Difluoromethyl)-5- | |
| | (isoquinolin-7-ylmethoxy)-2,2-dimethyl-4H- | |
| | benzo[d][1,3]dioxin-4-one (Intermediate | |
| | B10-2) | |
| B11-5 | 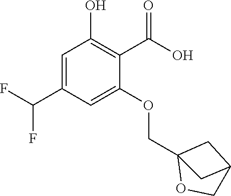 [SMILES: O=C(O)c1c(O)cc(C(F)F)cc1OCC12CC(CO1)C2] | ¹H NMR (400 MHz, DMSO) 1.40-1.37 (m, 2H), 1.84-1.81 (m, 2H), 2.93-2.90 (m, 1H), 3.69 (s, 2H), 4.24-4.23 (m, 2H), 6.67 (s, 1H), 6.73 (s, 1H), 6.91 (t, J = 55.8 Hz, 1H), 11.88 (s, 1H). 1 exchangeable proton not visible. MS: ES+ 301.1 (M + 1). |
| | 2-((2-Oxabicyclo[2.1.1]hexan-1- | |
| | yl)methoxy)-4-(difluoromethyl)-6- | |
| | hydroxybenzoic acid | |
| | Prepared from 5-((2- | |
| | oxabicyclo[2.1.1]hexan-1-yl)methoxy)-7- | |
| | (difluoromethyl)-2,2-dimethyl-4H- | |
| | benzo[d][1,3]dioxin-4-one (Intermediate | |
| | B10-3) | |

### Intermediate C 2-(Benzyloxy)-6-hydroxy-4-methoxybenzoic acid

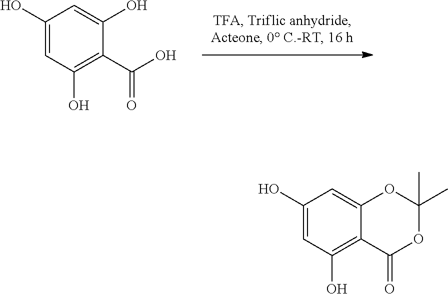

To a stirred solution of 2,4,6-trihydroxybenzoic acid (5 g, 29.41 mmol, 1 eq.) in TFA (38 mL) were added acetone (5 mL) and TFAA (28.5 mL) at 0° C. The reaction mixture was allowed warm to room temperature and stirred for 16 h. The resulting mixture was concentrated under vacuum and the crude material was taken up in saturated NaHCO₃ solution (200 mL). The mixture was extracted with EtOAc (3×170 mL) and the combined organic extracts were dried over Na₂SO₄, filtered and concentrated under vacuum. The crude material was triturated with diethyl ether (50 mL) and dried under high vacuum to yield the title compound as a brown solid (2.5 g, Yield: 40.0%)

¹H NMR (DMSO-d6, 400 MHz): δ 1.66 (s, 6H), 5.94 (d, J=2.0 Hz, 1H), 6.02 (d, J=2.0 Hz, 1H), 10.30 (s, 1H), 10.90 (s, 1H).

### Step 2: 5-Hydroxy-7-methoxy-2,2-dimethyl-4H-benzo[d][1,3]dioxin-4-one

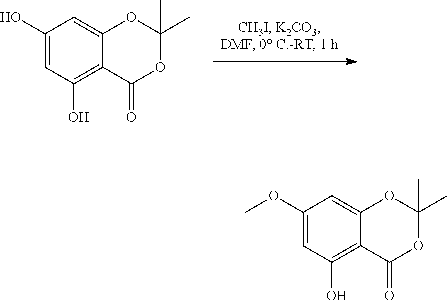

To a stirred solution of 5,7-dihydroxy-2,2-dimethyl-4H-benzo[d][1,3]dioxin-4-one (step 1) (1.9 g, 9.03 mmol, 1 eq.) in DMF (20 mL) were added K₂CO₃ (2.5 g, 18.07 mmol, 2.0 eq.) at 0° C. and the mixture was stirred for 10 mins. Methyl iodide (1.92 g, 13.55 mmol, 1.5 eq.) was added dropwise and the reaction mixture was allowed to warm to room temperature and stirred for 1 h. The resulting mixture was diluted with ice cold water (50 mL) and extracted with EtOAc (2×50 mL). The combined organic extracts were dried over Na₂SO₄, filtered and concentrated under vacuum. The crude material was purified by chromatography on silica (product eluted at 13% EtOAc in hexane) to yield the title compound (0.55 g, Yield: 27.0%).

¹H NMR (DMSO-d6, 400 MHz): δ 1.68 (s, 6H), 3.80 (s, 3H), 6.17 (d, J=2 Hz, 1H), 6.22 (d, J=2.4 Hz, 1H), 10.38 (s, 1H).

LCMS (Method A): 1.948 min, MS: ES+ 224.9 (M+1)

### Step 3: 5-(Benzyloxy)-7-methoxy-2,2-dimethyl-4H-benzo[d][1,3]dioxin-4-one

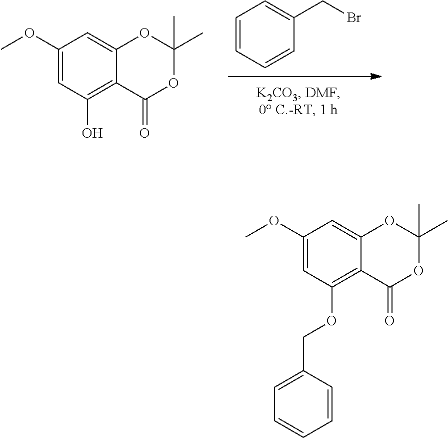

To a stirred solution of 5-hydroxy-7-methoxy-2,2-dimethyl-4H-benzo[d][1,3]dioxin-4-one (step 2)(0.55 g, 2.45 mmol, 1 eq.) in DMF (5.5 mL) were added K₂CO₃ (1.01 g, 7.36 mmol, 3.0 eq.) and the mixture was stirred at room temperature for 30 mins. The mixture was cooled to 0° C. and treated dropwise with benzyl bromide (0.83 g, 4.91 mmol, 2.0 eq.). The reaction mixture was allowed to warm to room temperature and stirred for 45 mins. The resulting mixture was poured into ice cold water (50 mL) and extracted with DCM (3×25 mL). The combined organic extracts were dried over Na₂SO₄, filtered and concentrated under vacuum. The crude material was purified by chromatography on silica (product eluted at 10% EtOAc in hexane) to yield the title compound (0.6 g, Yield: 77.0%).

¹H NMR (DMSO-d6, 400 MHz): δ 1.64 (s, 6H), 3.82 (s, 3H), 5.23 (s, 2H), 6.27 (d, J=2.4 Hz, 1H), 6.44 (d, J=2.4 Hz, 1H), 7.30-7.34 (m, 1H), 7.40 (t, J=7.2 Hz, 2H), 7.54 (d, J=7.2 Hz, 2H).

LCMS (Method A): 2.177 min, MS: ES+ 314.8 (M+1).

### Step 4: 2-(Benzyloxy)-6-hydroxy-4-methoxybenzoic acid

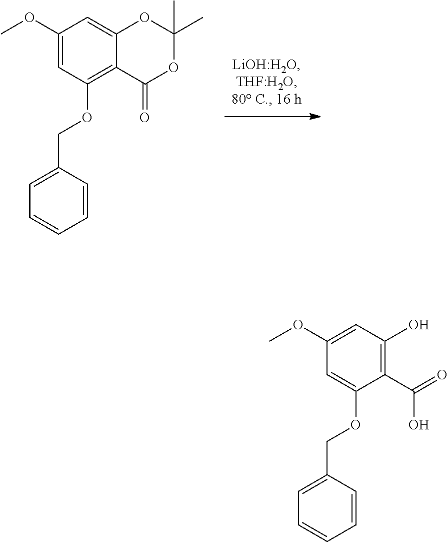

To a stirred solution of 5-(benzyloxy)-7-methoxy-2,2-dimethyl-4H-benzo[d][1,3]dioxin-4-one (step 3) (0.6 g, 1.91 mmol, 1 eq.) in THF:water (12 mL, 1:1) was added LiOH·H₂O (1.12 g, 26.75 mmol, 14 eq.) at room temperature. The reaction mixture was heated to 80° C. for 16 h. The resulting mixture was allowed to cool to room temperature, acidified with saturated KHSO₄ solution (50 mL) and extracted with EtOAc (2×55 mL). The combined organic extracts were dried over Na₂SO₄, filtered and concentrated under vacuum. The crude material was triturated with pentane to yield the title compound (0.4 g, Yield: 76.5%)

¹H NMR (DMSO-d6, 400 MHz): δ 3.75 (s, 3H), 5.15 (s, 2H), 6.09 (d, J=2.4 Hz, 1H), 6.17 (d, J=2 Hz, 1H), 7.31-7.33 (m, 1H), 7.37-7.40 (m, 3H), 7.49 (d, J=7.2 Hz, 2H), 12.63 (bs, 1H).

LCMS (Method A): 2.033 min, MS: ES+ 274.8 (M+1).

### Intermediate D 2-(Benzyloxy)-4-(difluoromethyl)-6-hydroxybenzoic acid

### Step 1: Methyl 4-bromo-3,5-dihydroxybenzoate

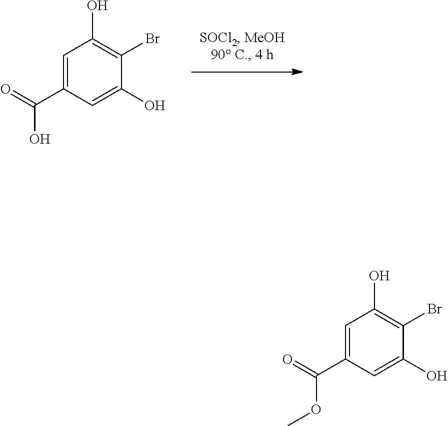

To a solution of 4-bromo-3,5-dihydroxybenzoic acid (50 g, 214.5 mmol, 1.0 eq.) in MeOH (300 mL) was added dropwise thionyl chloride (76.60 g, 644.0 mmol, 3 eq.) at 0° C. The reaction mixture was heated to 90° C. for 4 h. The resulting mixture was concentrated under vacuum. The crude material was triturated with diethyl ether (200 mL) followed by n-pentane (200 mL) and dried under high vacuum to yield the title compound as a white solid (56 g, Yield: Quantitative). The crude material was used in the next step without further purification.

¹H NMR (DMSO-d6, 400 MHz): δ 3.98-4.05 (m, 3H), 7.03 (s, 2H), 10.57 (s, 2H).

LCMS (Method A): 1.170 min, MS: ES− 244.83 (M−2).

### Step 2: Methyl 4-bromo-3,5-bis(tosyloxy)benzoate

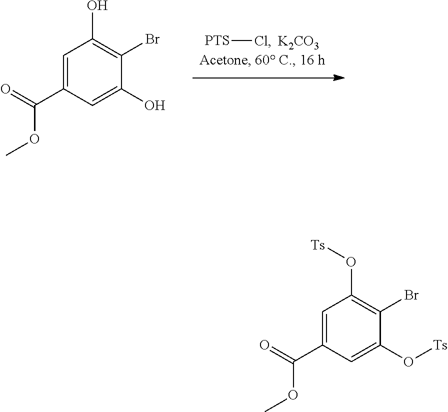

To a stirred solution of methyl 4-bromo-3,5-dihydroxybenzoate (step 1) (56 g, 226.0 mmol, 1.0 eq.) in acetone (500 mL) were added TsCl (130 g, 680.0 mmol, 3 eq.) and K₂CO₃ (94 g, 680 mmol, 3 eq.) at room temperature. The reaction mixture was heated to 60° C. for 16 h. The resulting mixture was concentrated under vacuum and the crude material was diluted with ice cold water (3 L) and extracted with EtOAc (3×3 L). The combined organic extracts were concentrated under vacuum to yield the title compound as an off-white solid (98 g, Yield: 78%). The crude material was used in the next step without further purification.

¹H NMR (DMSO-d6, 400 MHz): δ 2.44-2.51 (m, 6H), 3.86 (s, 3H), 7.49-7.51 (m, 4H), 7.63 (s, 2H), 7.75-7.77 (m, 4H).

LCMS (Method A): 2.636 min, MS: ES+ 554.89 (M−2).

### Step 3: 2-Bromo-5-(hydroxymethyl)-1,3-phenylene bis(4-m ethylbenzenesulfonate)

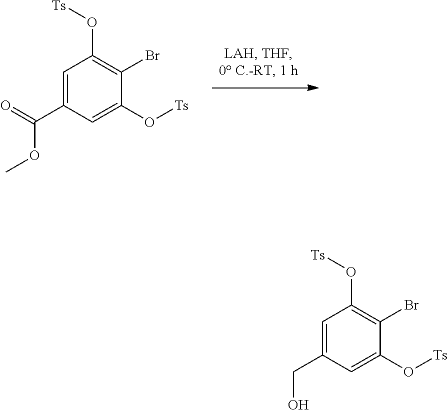 [SMILES: COC(=O)c1cc(OS(=O)(=O)c2ccc(C)cc2)c(Br)c(OS(=O)(=O)c2ccc(C)cc2)c1 ; Cc1ccc(S(=O)(=O)Oc2cc(CO)cc(OS(=O)(=O)c3ccc(C)cc3)c2Br)cc1]

A stirred solution of methyl 4-bromo-3,5-bis(tosyloxy)benzoate (step 2) (60 g, 108.0 mmol, 1.0 eq.) in THF (600 mL) was cooled to 0° C. Lithium aluminium hydride (1M in THF) (162 mL, 162 mmol, 1.5 eq.) was added dropwise at 0° C. and the mixture was allowed to warm to room temperature and stirred for 30 mins. The resulting mixture was poured into ice cold saturated NH₄Cl solution (1000 mL) and filtered through Celite® (filter material), washing through with EtOAc (1000 mL) to remove the gelatinous precipitate. The filtrate was further extracted with EtOAc (3×1000 mL) and the combined organic extracts were dried over Na₂SO₄, filtered and concentrated under vacuum to yield the title compound as a yellow oil (47 g, Yield: 82%). The crude material was used in the next step without further purification.

¹H NMR (DMSO-d6, 400 MHz): δ 2.43-2.56 (m, 6H), 4.37-4.50 (m, 2H), 5.40-5.43 (m, 1H), 7.05 (s, 2H), 7.47-7.52 (m, 4H), 7.68-7.75 (m, 4H).

### Step 4: 2-Bromo-5-formyl-1,3-phenylene bis(4-methyl benzenesulfonate)

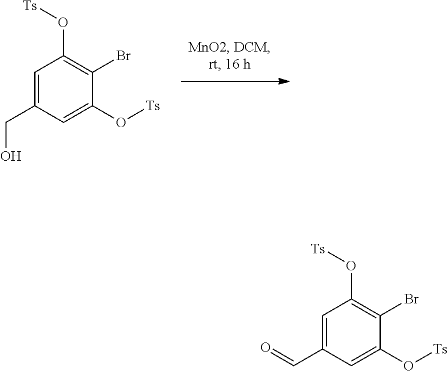 [SMILES: Cc1ccc(S(=O)(=O)Oc2cc(CO)cc(OS(=O)(=O)c3ccc(C)cc3)c2Br)cc1 ; Cc1ccc(S(=O)(=O)Oc2cc(C=O)cc(OS(=O)(=O)c3ccc(C)cc3)c2Br)cc1]

To a stirred solution of 2-bromo-5-(hydroxymethyl)-1,3-phenylene bis(4-methylbenzenesulfonate) (step 3) (47 g, 1.0 eq.) in DCM (470 mL) was added MnO₂ (470 g, 10% W/W) at room temperature. The reaction mixture was stirred at room temperature for 16 h. The resulting mixture was filtered, washing through with DCM (3×1 L). The combined filtrate was concentrated under vacuum and the crude material was purified by chromatography on silica (product was eluted in 10% EtOAc in Hexane) to yield the title compound as a yellow oil (19 g, Yield: 40%).

¹H NMR (DMSO-d6, 400 MHz): 2.43-2.56 (m, 6H), 7.11 (t, J=2 Hz, 1H), 7.48-7.50 (m, 4H), 7.55-7.56 (m, 2H), 7.71-7.78 (m, 4H), 9.87 (s, 1H).

### Step 5: 2-Bromo-5-(difluoromethyl)-1,3-phenylene bis(4-methylbenzenesulfonate)

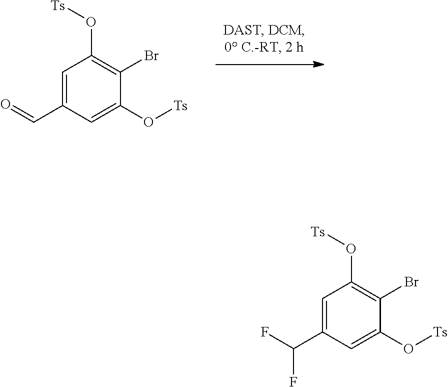 [SMILES: Cc1ccc(S(=O)(=O)Oc2cc(C=O)cc(OS(=O)(=O)c3ccc(C)cc3)c2Br)cc1 ; Cc1ccc(S(=O)(=O)Oc2cc(C(F)F)cc(OS(=O)(=O)c3ccc(C)cc3)c2Br)cc1]

To a stirred solution of 2-bromo-5-formyl-1,3-phenylene bis(4-methylbenzenesulfonate) (step 4) (19 g, 36.19 mol, 1.0 eq.) in DCM (190 mL) was added DAST (29.13 g, 180 mml, 5 eq.) at 0° C. The reaction mixture was allowed to warm to room temperature and stirred for 1.5 h. The resulting mixture was poured into ice cold saturated NaHCO₃ solution (1000 mL) and extracted with DCM (3×1000 mL). The combined organic extracts were dried over Na₂SO₄, filtered and concentrated under vacuum. The crude material was purified by chromatography on silica (product was eluted in 10% EtOAc in Hexane) to yield the title compound (11.5 g, Yield: Quantitative).

¹H NMR (DMSO-d6, 400 MHz): 2.43-2.51 (m, 6H), 6.84-6.96 (m, 1H), 7.11-7.23 (m, 2H), 7.48-7.55 (m, 4H), 7.69-7.76 (m, 4H).

### Step 6: 4-(Difluoromethyl)-2,6-bis(tosyloxy)benzoic acid

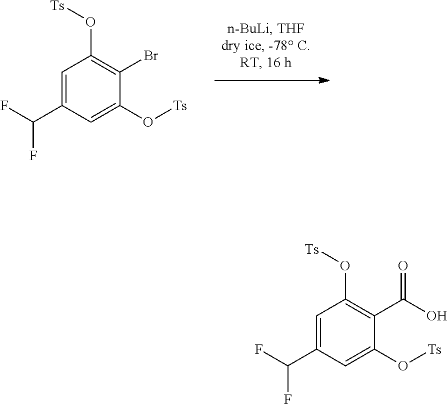 [SMILES: Cc1ccc(S(=O)(=O)Oc2cc(C(F)F)cc(OS(=O)(=O)c3ccc(C)cc3)c2Br)cc1 ; Cc1ccc(S(=O)(=O)Oc2cc(C(F)F)cc(OS(=O)(=O)c3ccc(C)cc3)c2C(=O)O)cc1]

To a stirred solution 2-bromo-5-(difluoromethyl)-1,3-phenylene bis(4-methylbenzenesulfonate) (step 5) (5.5 g, 10.05 mmol, 1.0 eq.) in THF (55 mL), cooled to −78° C. was added dropwise n-BuLi (1.6M in Hexane) (13.13 mL, 2 eq.) and stirred for 30 mins. Dry ice (small piece) was added to the reaction mixture at −78° C. The mixture was allowed to warm to room temperature and stirred for 16 h. The resulting mixture was poured into ice cold NH₄Cl solution (500 mL) and extracted with 10% MeOH in DCM (3×500 mL). The combined organic extracts were dried over Na₂SO₄, filtered, concentrated under vacuum. The crude material was purified by chromatography on silica (product was eluted in 10% MeOH in DCM) material to yield the title compound as a yellow solid (7.5 g, Yield: 40%).

¹H NMR (DMSO-d6, 400 MHz): δ 2.42 (s, 6H), 6.94-7.21 (m, 1H), 7.34 (m, 2H), 7.45-7.50 (m, 4H), 7.72-7.79 (m, 4H).

### Step 7: 4-(Difluoromethyl)-2,6-dihydroxybenzoic acid

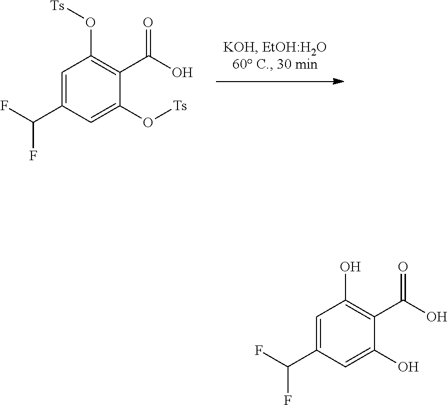 [SMILES: Cc1ccc(S(=O)(=O)Oc2cc(C(F)F)cc(OS(=O)(=O)c3ccc(C)cc3)c2C(=O)O)cc1 ; O=C(O)c1c(O)cc(C(F)F)cc1O]

To a stirred solution 4-(difluoromethyl)-2,6-bis(tosyloxy)benzoic acid (step 6) (7.5 g, 15.0 mmol, 1.0 eq.) in EtOH:H₂O (2:1) (100 mL) was added KOH (32.81 g, 58.5 mmol, 40 eq.) and the mixture was heated to 60° C. for 16 h. The resulting mixture was poured into ice cold water (200 mL), acidified with dilute HCl and neutralized with sat. NaHCO₃ solution. The mixture was extracted with EtOAc (3×200 mL) followed by 10% MeOH:DCM (3×100 mL). The combined organic extracts were dried over Na₂SO₄, filtered, and concentrated under vacuum. The crude material was diluted with EtOAc (300 mL) and washed with saturated NaHCO₃ solution (200 mL). The aqueous layer was acidified with dilute HCl and extracted with EtOAc (3×300 mL). The combined organic extracts were dried over Na₂SO₄, filtered and concentrated under vacuum to yield the title compound as an off-white solid (2.0 g, Yield: 67%). The crude material was used in the next step without further purification.

¹H NMR (DMSO-d6, 400 MHz): δ 6.19-6.33 (m, 2H), 6.77-6.93 (m, 1H), 14.94 (s, 2H).

LCMS (Method A): 1.341 min, MS: ES+ 203.0 (M−1).

### Step 8: 7-(Difluoromethyl)-5-hydroxy-2,2-dimethyl-4H-benzo[d][1,3]dioxin-4-one

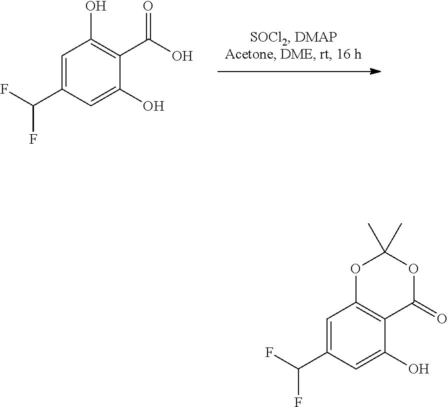 [SMILES: O=C(O)c1c(O)cc(C(F)F)cc1O ; CC1(C)OC(=O)c2c(O)cc(C(F)F)cc2O1]

To a stirred solution 4-(difluoromethyl)-2,6-dihydroxybenzoic acid (step 7) (2 g, 9.80 mmol, 1.0 eq.) in DMF (20 mL) were added acetone (5.68 g, 98.0 mmol, 10 eq.) followed by DMAP (0.12 g, 0.98 mmol, 0.1 eq.). The mixture was cooled to 0° C., treated dropwise with thionyl chloride (3.5 g, 29.41 mmol, 3 eq.) and allowed to warm to room temperature, stirring for 16 h. The resulting mixture was poured into ice-cooled water (100 mL), neutralized with sat. NaHCO₃ and extracted with EtOAc (3×100 mL). The combined organic extracts were dried over Na₂SO₄, filtered and concentrated under vacuum. The crude material was purified by chromatography on silica (product was eluted in 10% EtOAc in Hexane) to yield the title compound as an off-white solid (0.8 g, Yield: 33%).

¹H NMR (DMSO-d6, 400 MHz): δ 1.68-1.71 (m, 6H), 6.71 (s, 1H), 6.83-6.84 (m, 1H), 6.98-7.12 (m, 1H), 10.65 (s, 1H).

### Step 9: 5-(Benzyloxy)-7-(difluoromethyl)-2,2-dimethyl-4H-benzo[d][1,3]dioxin-4-one

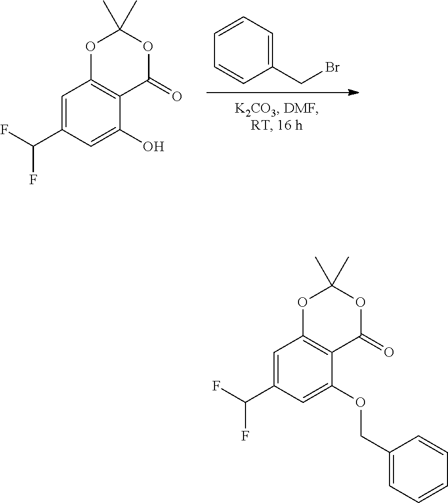 [SMILES: CC1(C)OC(=O)c2c(O)cc(C(F)F)cc2O1 ; BrCc1ccccc1 ; CC1(C)OC(=O)c2c(OCc3ccccc3)cc(C(F)F)cc2O1]

To a stirred solution 7-(difluoromethyl)-5-hydroxy-2,2-dimethyl-4H-benzo[d][1,3]dioxin-4-one (step 8) (0.8 g, 3.27 mmol, 1.0 eq.) in DMF (8 mL) at room temperature was added K₂CO₃ (1.35 g, 9.80 mmol, 3 eq.) and the mixture was stirred for 10 mins. Benzyl bromide (1.12 g, 6.50 mmol, 1.0 eq.) was added and stirring continued at room temperature for 16 h. The resulting mixture was poured into ice cold water (100 mL), neutralized with saturated NaHCO₃ solution and extracted with EtOAc (3×100 mL). The combined organic extracts were dried over Na₂SO₄, filtered and concentrated under vacuum. The crude material was purified by chromatography on silica (product was eluted in 20% EtOAc in hexane) to yield the title compound (0.9 g, Yield: 82%).

¹H NMR (DMSO-d6, 400 MHz): δ 1.68-1.71 (m, 6H), 5.32 (s, 2H), 6.87-6.89 (m, 1H), 7.03 (s, 1H), 7.13-7.16 (m, 1H), 7.31-7.35 (m, 2H), 7.41 (t, J=7.2 Hz, 2H), 7.55-7.57 (m, 2H).

### Step 10: 2-(Benzyloxy)-4-(difluoromethyl)-6-hydroxybenzoic acid

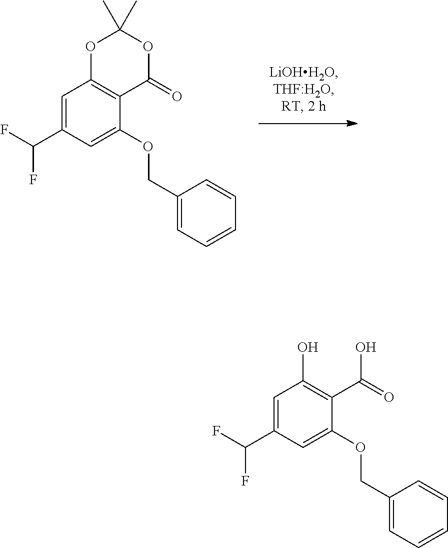 [SMILES: CC1(C)OC(=O)c2c(OCc3ccccc3)cc(C(F)F)cc2O1 ; O=C(O)c1c(O)cc(C(F)F)cc1OCc1ccccc1]

To a stirred solution 5-(benzyloxy)-7-(difluoromethyl)-2,2-dimethyl-4H-benzo[d][1,3]dioxin-4-one (step 9) (0.9 g, 2.69 mmol, 1.0 eq.) in THF:water (2:1) (15 mL) was added LiOH·H₂O (0.33 g, 8.00 mmol, 3 eq.) and the mixture was stirred at room temperature for 2 h. The resulting mixture was poured into ice cold water (100 mL), neutralized with dilute HCl and extracted with EtOAc (3×100 mL). The combined organic extracts were dried over Na₂SO₄, filtered and concentrated under vacuum to yield the title compound (0.9 g, Yield: 76%). The crude material was used in the next step without further purification.

¹H NMR (DMSO-d6, 400 MHz): δ 3.17 (s, 1H), 5.15 (s, 2H), 6.69 (s, 1H), 6.78 (s, 1H), 6.92 (s, 1H), 7.31-7.44 (m, 6H), 10.47-10.52 (m, 1H), 12.95 (bs, 1H).

LCMS (Method A): 2.020 min, MS: ES+ 293.2 (M−1).

### Intermediate DA 4-(Difluoromethyl)-2-hydroxy-6-((3-methoxybenzyl)oxy)benzoic acid

### Step 1: 7-(Difluoromethyl)-5-((3-methoxybenzyl)oxy)-2,2-dimethyl-4H-benzo[d][1,3]dioxin-4-one

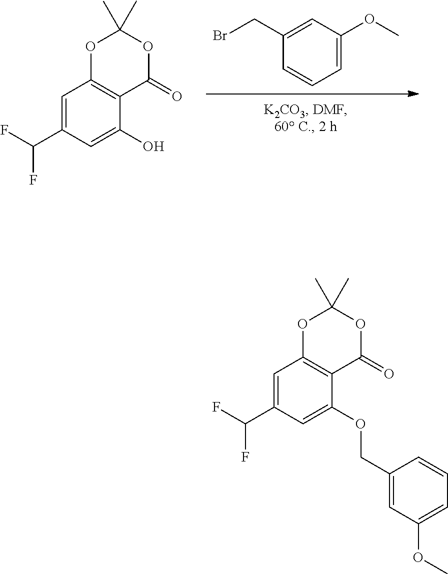

The title compound was prepared from 7-(difluoromethyl)-5-hydroxy-2,2-dimethyl-4H-benzo[d][1,3]dioxin-4-one (Intermediate D step 8) and 1-(bromomethyl)-3-methoxybenzene (CAS: 874-98-6) analogously to Intermediate D step 9.

¹H NMR (DMSO-d6, 400 MHz): δ 1.69 (s, 6H), 3.77 (s, 3H), 5.30 (s, 2H), 6.87-6.91 (m, 2H), 7.03 (s, 1H), 7.09-7.17 (m, 2H), 7.23 (s, 1H), 7.32 (t, J=7.6 Hz, 15.6 Hz, 1H).

### Step 2: 4-(Difluoromethyl)-2-hydroxy-6-((3-methoxybenzyl)oxy)benzoic acid

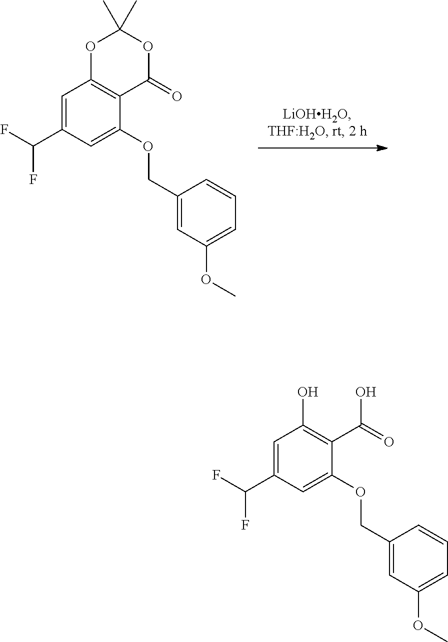 [SMILES: COc1cccc(COc2cc(C(F)F)cc3c2C(=O)OC(C)(C)O3)c1 ; COc1cccc(COc2cc(C(F)F)cc(O)c2C(=O)O)c1]

The title compound was prepared from 7-(difluoromethyl)-5-((3-methoxybenzyl)oxy)-2,2-dimethyl-4H-benzo[d][1,3]dioxin-4-one (step 1) and LiOH·H₂O analogously to Intermediate D step 10.

¹H NMR (DMSO-d6, 400 MHz): δ 3.75 (s, 3H), 5.14 (s, 2H), 6.69 (s, 1H), 6.77-6.79 (m, 1H), 6.82-6.89 (m, 1H), 6.93-7.07 (m, 3H), 7.3 (t, J=8.0 Hz, 16 Hz, 1H), 10.46-10.48 (m, 1H), 12.99 (s, 1H).

### Intermediate DB 4-(Difluoromethyl)-2-hydroxy-6-(pyridin-2-ylmethoxy)benzoic acid

### Step 1: 7-(Difluoromethyl)-2,2-dimethyl-5-(pyridin-2-ylmethoxy)-4H-benzo[d][1,3]dioxin-4-one

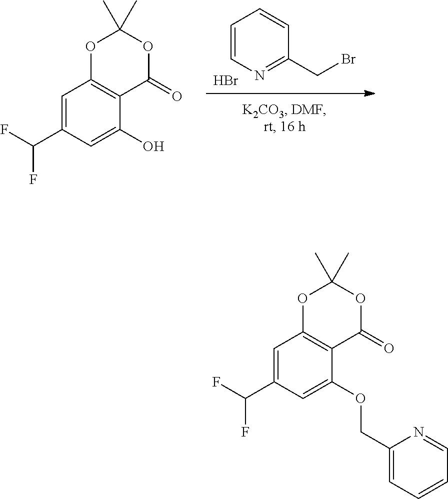 [SMILES: CC1(C)OC(=O)c2c(O)cc(C(F)F)cc2O1 ; Br ; BrCc1ccccn1 ; CC1(C)OC(=O)c2c(OCc3ccccn3)cc(C(F)F)cc2O1]

The title compound was prepared from 7-(difluoromethyl)-5-hydroxy-2,2-dimethyl-4H-benzo[d][1,3]dioxin-4-one (Intermediate D step 8) and 2-(bromomethyl)pyridine hydrobromide (CAS: 31106-82-8) analogously to Intermediate D step 9.

1H NM R (DMSO-d6, 400 MHz): δ 1.70 (s, 6H), 5.36 (s, 2H), 6.89 (s, 1H), 7.03 (s, 1H), 7.16 (d, J=3.6 Hz, 1H), 7.38-7.34 (m, 1H), 7.82 (d, J=7.6 Hz, 1H), 7.90-7.81 (m, 1H), 8.59-8.58 (m, 1H).

LCMS (Method A): 1.714 min, MS: ES+ 335.8 (M+1).

### Step 2: 4-(Difluoromethyl)-2-hydroxy-6-(pyridin-2-ylmethoxy)benzoic acid

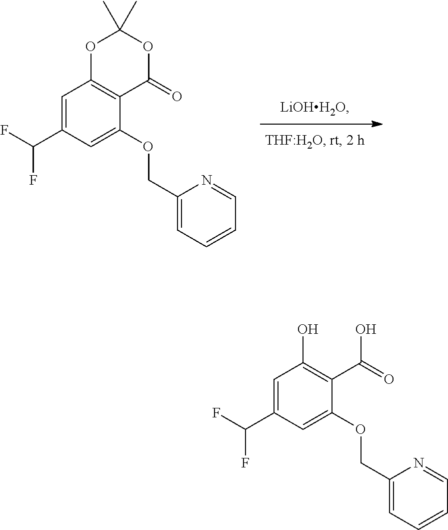 [SMILES: CC1(C)OC(=O)c2c(OCc3ccccn3)cc(C(F)F)cc2O1 ; O=C(O)c1c(O)cc(C(F)F)cc1OCc1ccccn1]

The title compound was prepared from 7-(difluoromethyl)-2,2-dimethyl-5-(pyridin-2-ylmethoxy)-4H-benzo[d][1,3]dioxin-4-one (step 1) and LiOH·H₂O analogously to Intermediate D step 10.

1H NMR (DMSO-d6, 400 MHz): δ 5.20 (s, 2H), 6.63 (d, J=9.6 Hz, 2H), 6.87 (s, 1H), 7.34 (t, J=6.8 Hz, J=12.4 Hz, 1H), 7.71 (d, J=8.0 Hz, 1H), 7.85 (t, J=9.2 Hz, J=16.8 Hz, 1H), 8.56 (d, J=4.4 Hz, 1H).

LCMS (Method A): 1.189 min, MS: ES+ 295.79 (M+1).

### Intermediate DC 4-(Difluoromethyl)-2-hydroxy-6-((4-methoxybenzyl)oxy)benzoic acid

### Step 1: 7-(Difluoromethyl)-5-((4-methoxybenzyl)oxy)-2,2-dimethyl-4H-benzo[d][1,3]dioxin-4-one

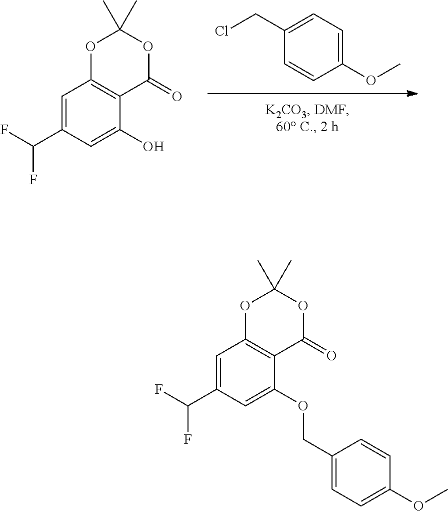

The title compound was prepared from 7-(difluoromethyl)-5-hydroxy-2,2-dimethyl-4H-benzo[d][1,3]dioxin-4-one (Intermediate D step 8) and 1-(chloromethyl)-4-methoxybenzene (CAS: 824-94-2) analogously to Intermediate D step 9.

¹H NMR (DMSO-d6, 400 MHz): δ 1.68 (s, 6H), 3.73-3.77 (m, 4H), 2.23 (s, 2H), 6.85-6.89 (m, 2H), 6.97 (d, J=8.4 Hz, 2H), 7.03 (s, 1H), 7.13-7.16 (m, 1H), 7.22 (d, J=8.8 Hz, 2H).

### Step 2: 4-(Difluoromethyl)-2-hydroxy-6-((4-methoxybenzyl)oxy)benzoic acid

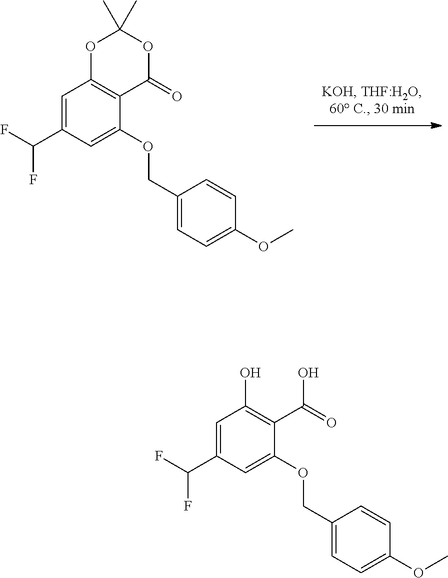 [SMILES: COc1ccc(COc2cc(C(F)F)cc3c2C(=O)OC(C)(C)O3)cc1 ; COc1ccc(COc2cc(C(F)F)cc(O)c2C(=O)O)cc1]

To a stirred solution of 7-(difluoromethyl)-5-((4-methoxybenzyl)oxy)-2,2-dimethyl-4H-benzo[d][1,3]dioxin-4-one (0.3 g, 0.82 mmol, 1.0 eq) in THF:H2O (2 mL: 1 mL) (2:1) was added KOH (0.992 g, 17.71 mmol, 20 eq) at room temperature. The resulting mixture was heated to 60° C. and stirred for 30 min. The reaction mixture was concentrated under vacuum. The crude material diluted with ice cold water (50 mL), pH adjusted ~7 using saturated citric acid solution and extracted with diethyl ether (3×50 mL). The combined organic layers were dried over Na₂SO₄, filtered and concentrated under vacuum at room temperature (without applying heating) yielding 4-(difluoromethyl)-2-hydroxy-6-((4-methoxybenzyl)oxy)benzoic acid as an off white solid (0.25 g, Yield: 93%).

1H NM R (DMSO-d6, 400 MHz): δ 3.76 (s, 3H), 5.07 (s, 2H), 6.68 (s, 1H), 6.79 (s, 1H), 6.93-6.96 (m, 3H), 7.35 (d, J=8.8 Hz, 2H), 10.42 (s, 1H), 12.96 (s, 1H).

LCMS (Method N): 1.742 min, MS:ES− 322.9 (M−1).

### Intermediate E 2-(Benzyloxy)-4-fluoro-6-hydroxybenzoic acid

### Step 1: 4-Fluoro-2,6-dihydroxybenzoic acid

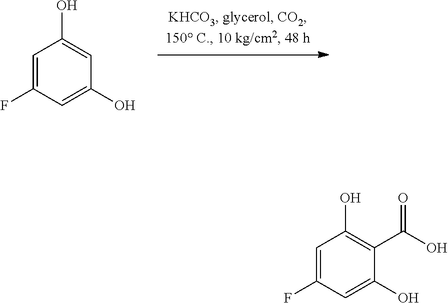 [SMILES: Oc1cc(O)cc(F)c1 ; O=C(O)c1c(O)cc(F)cc1O]

To a stirred solution of 5-fluorobenzene-1,3-diol (CAS 75996-29-1) (1.0 g, 7.80 mmol, 1 eq.) in glycerol (10 mL) at room temperature was added KHCO₃ (3.28 g, 32.78 mmol, 4.2 eq.) and the mixture was cooled to −78° C. Dry ice (10 g, w/w) was added and the mixture was placed under a 10 kg CO₂ pressure in a hydrogenator at room temperature. The reaction mixture was heated to 150° C. and stirred for 48 h. The resulting mixture was diluted with water (50 mL), acidified with 1N HCl and extracted with EtOAc (2×75 mL). The combined organic extracts were dried over Na₂SO₄, filtered and concentrated under vacuum to yield the title compound as an orange liquid (1.1 g, Yield: 81%). The crude material was used in the next step without further purification.

¹H NMR (DMSO-d6, 400 MHz): δ 5.92-5.96 (m, 2H).

LCMS (Method A): 1.259 min, MS: ES− 170.93 (M−1).

### Step 2: 7-Fluoro-5-hydroxy-2,2-dimethyl-4H-benzo[d][1,3]dioxin-4-one

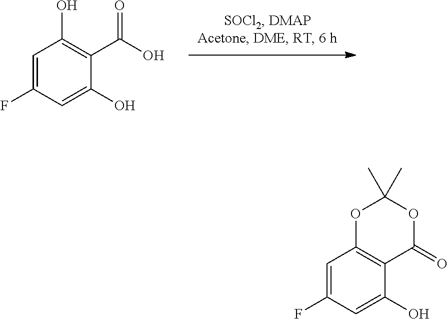

To a stirred solution of 4-fluoro-2,6-dihydroxybenzoic acid (step 1) (1.1 g, 6.39 mmol, 1 eq.) in DME (11 mL) were added acetone (4.8 mL, 63.9 mmol, 10 eq.) and DMAP (0.078 g, 0.63 mmol, 0.1 eq.) at room temperature. The mixture was cooled to 0° C., treated dropwise with thionyl chloride (1.4 mL, 19.2 mmol, 3 eq.) and allowed to warm to room temperature stirring for 6 h. The reaction was quenched with saturated NaHCO₃ solution (50 mL) and the mixture was extracted with EtOAc (3×30 mL). The combined organic extracts were dried over Na₂SO₄, filtered and concentrated under vacuum. The crude material was purified by chromatography on silica (product eluted at 1% EtOAc in hexane) to yield the title compound as a light yellow solid (0.21 g, Yield: 17.0%)

¹H NMR (DMSO-d6, 400 MHz): δ 1.70 (s, 6H), 6.51-6.57 (m, 2H), 10.72 (s, 1H).

### Step 3: 5-(Benzyloxy)-7-fluoro-2,2-dimethyl-4H-benzo[d][1,3]dioxin-4-one

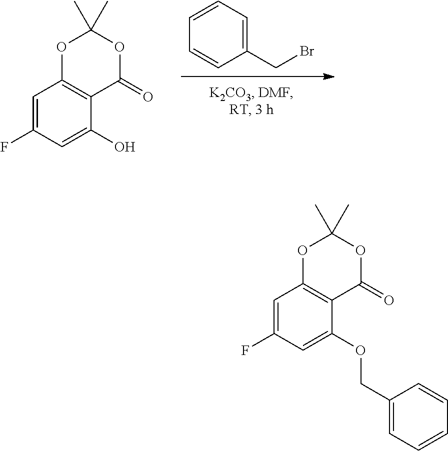 [SMILES: CC1(C)OC(=O)c2c(O)cc(F)cc2O1 ; BrCc1ccccc1 ; CC1(C)OC(=O)c2c(OCc3ccccc3)cc(F)cc2O1]

To a stirred solution of 7-fluoro-5-hydroxy-2,2-dimethyl-4H-benzo[d][1,3]dioxin-4-one (step 2) (0.2 g, 0.94 mmol, 1 eq.) in DMF (2 mL) were added K₂CO₃ (0.39 g, 2.83 mmol, 3.0 eq.) and the mixture was stirred at room temperature for 15 mins. The mixture was cooled to 0° C., treated dropwise with benzyl bromide (0.2 mL, 1.88 mmol, 2.0 eq.) and allowed to warm to room temperature stirring for 3 h. The resulting mixture was poured into ice cold water (20 mL) and a solid precipitated. The solid was collected by filtration and washed with hexane (20 mL). The crude material was triturated with n-pentane (3×10 mL) and dried under vacuum to yield the title compound as a brown solid (0.18 g, Yield: 63.2%).

¹H NMR (DMSO-d6, 400 MHz): δ 1.67 (s, 6H), 5.27 (s, 2H), 6.62, 6.65 (dd, J=4 Hz, 1H), 6.88, 6.91 (dd, J=4 Hz, 1H), 7.34-7.36 (m, 1H), 7.40-7.44 (m, 2H), 7.54 (d, J=4 Hz, 2H).

### Step 4: 2-(Benzyloxy)-4-fluoro-6-hydroxybenzoic acid

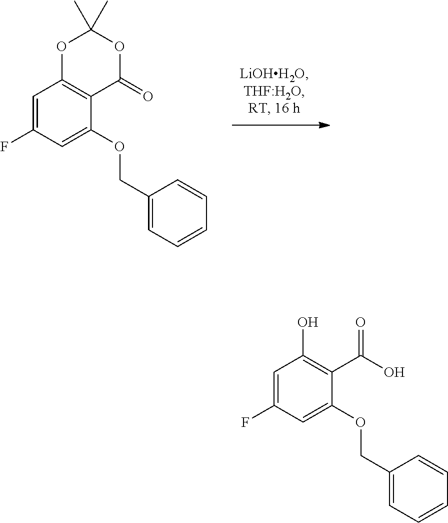 [SMILES: CC1(C)OC(=O)c2c(OCc3ccccc3)cc(F)cc2O1 ; O=C(O)c1c(O)cc(F)cc1OCc1ccccc1]

To a stirred solution of 5-(benzyloxy)-7-fluoro-2,2-dimethyl-4H-benzo[d][1,3]dioxin-4-one (step 3) (0.18 g, 0.59 mmol, 1 eq.) in THE (18 mL):water (9 mL) was added LiOH·H₂O (0.074 g, 1.78 mmol, 3 eq.) and the reaction mixture was stirred at room temperature for 16 h. The resulting mixture was diluted with water (20 mL), acidified with saturated KHSO₄ solution (10 mL) and extracted with EtOAc (2×30 mL). The combined organic extracts were dried over Na₂SO₄, filtered and concentrated under vacuum to yield the title compound as a light brown solid (0.15 g, Yield: 96.1%).

¹H NMR (DMSO-d6, 400 MHz): δ 5.13 (s, 2H), 6.31 (d, J=2 Hz, 1H), 6.48, 6.51 (dd, J=4 Hz, 1H), 7.33 (t, J=8 Hz, 1H), 7.37-7.46 (m, 4H), 12.31 (s, 1H).

LCMS (Method A): 2.033 min, MS: ES− 260.9 (M−1).

### Intermediate F 2-(Benzyloxy)-4-chloro-6-hydroxybenzoic acid

### Step 1: 7-Chloro-5-hydroxy-2,2-dimethyl-4H-benzo[d][1,3]dioxin-4-one

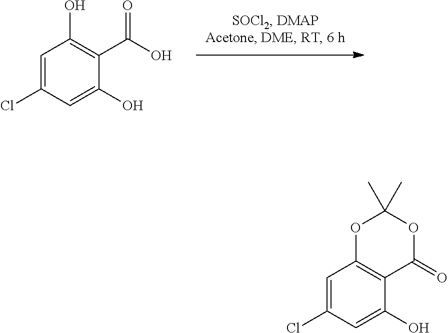

To a stirred solution of 4-chloro-2,6-dihydroxybenzoic acid (CAS 87006-36-8) (7.0 g, 37.23 mmol, 1 eq.) in DME (70 mL) were added acetone (27.58 mL, 372.3 mmol, 10 eq.) and DMAP (0.45 g, 3.72 mmol, 0.1 eq.) at room temperature. The mixture was cooled to 0° C. and treated dropwise with SOCl₂ (8.0 mL, 111.7 mmol, 3 eq.). The mixture was allowed to warm to room temperature and stirred for 6 h. The reaction was quenched with saturated NaHCO₃ solution (100 mL) and extracted with EtOAc (2×100 mL). The combined organic extracts were dried over Na₂SO₄, filtered, and concentrated under vacuum. The crude material was purified by chromatography on silica (product eluted at 4% EtOAc in hexane) to yield the title compound as a light brown solid (3.3 g, Yield: 30.87%)

¹H NMR (DMSO-d6, 400 MHz): δ 1.69 (s, 6H), 6.71 (d, J=4 Hz, 1H), 6.76 (d, J=4 Hz, 1H), 10.69 (s, 1H).

### Step 2: 5-(Benzyloxy)-7-chloro-2,2-dimethyl-4H-benzo[d][1,3]dioxin-4-one

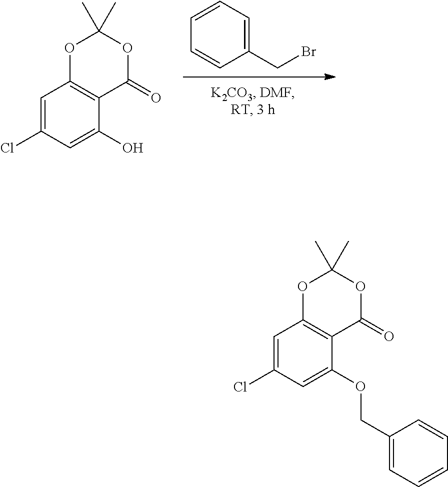 [SMILES: CC1(C)OC(=O)c2c(O)cc(Cl)cc2O1 ; BrCc1ccccc1 ; CC1(C)OC(=O)c2c(OCc3ccccc3)cc(Cl)cc2O1]

To a stirred solution of 7-chloro-5-hydroxy-2,2-dimethyl-4H-benzo[d][1,3]dioxin-4-one (step 1) (2.6 g, 11.4 mmol, 1 eq.) in DMF (26 ml) was added K₂CO₃ (4.72 g, 34.21 mmol, 3.0 eq.) and the mixture was stirred at room temperature for 15 mins. The mixture was cooled to 0° C. and treated dropwise with benzyl bromide (2.7 mL, 22.80 mmol, 2.0 eq.). The mixture was allowed to warm to room temperature and stirred for 3 h. The resulting mixture was poured into ice cold water (50 mL) and a solid precipitated. The solid was collected by filtration and washed with hexane (60 mL). The crude material was triturated with n-pentane (3×10 mL) and concentrated under vacuum to yield the title compound as an off-white solid (5.3 g, Yield: quantitative).

¹H NMR (DMSO-d6, 400 MHz): δ 1.67 (s, 6H), 5.29 (s, 2H), 6.86 (d, J=1.6 Hz, 1H), 7.07 (d, J=4 Hz, 1H), 7.32-7.36 (m, 1H), 7.40-7.44 (m, 2H), 7.54 (d, J=8 Hz, 2H).

### Step 3: 2-(Benzyloxy)-4-chloro-6-hydroxybenzoic acid

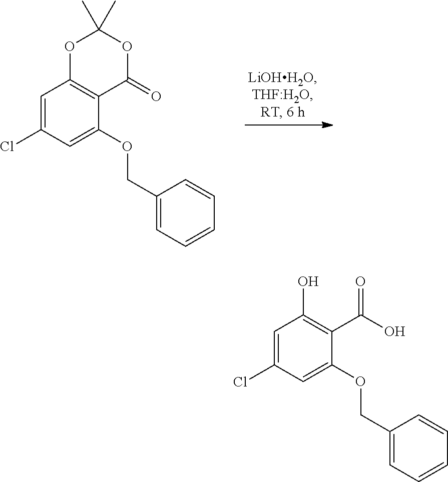 [SMILES: CC1(C)OC(=O)c2c(OCc3ccccc3)cc(Cl)cc2O1 ; O=C(O)c1c(O)cc(Cl)cc1OCc1ccccc1]

To a stirred solution of 5-(benzyloxy)-7-chloro-2,2-dimethyl-4H-benzo[d][1,3]dioxin-4-one (step 2) (5.0 g, 15.72 mmol, 1 eq.) in THE (50 mL):water (25 mL) was added LiOH·H₂O (1.97 g, 47.16 mmol, 3 eq.) and the reaction mixture was stirred at room temperature for 6 h. The resulting mixture was diluted with water (50 mL), acidified by saturated KHSO₄ solution (30 mL) and extracted with EtOAc (3×50 mL). The combined organic extracts were dried over Na₂SO₄, filtered and concentrated under vacuum to yield the title compound as an off-white solid (2.3 g, Yield: 50.3%).

¹H NMR (DMSO-d6, 400 MHz): δ 5.15 (s, 2H), 6.5 (d, J=1.6 Hz, 1H), 6.70 (d, J=1.6 Hz, 1H), 7.32-7.39 (m, 5H), 10.82 (bs, 1H), 12.97 (bs, 1H).

### Intermediate G (R)-6-Methoxy-N-(tetrahydrofuran-3-yl)isoindolin-4-amine hydrochloride

### Step 1: tert-Butyl 4-bromo-6-methoxyisoindoline-2-carboxylate

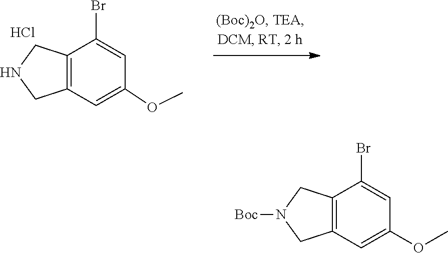 [SMILES: COc1cc(Br)c2c(c1)CNC2 ; Cl ; COc1cc(Br)c2c(c1)CN(C(=O)OC(C)(C)C)C2]

To a stirred solution of 4-bromo-6-methoxyisoindoline hydrochloride (CAS142740090-5 free base) (1.6 g, 6.0 mmol, 1 eq.) in DCM (20 mL) was added TEA (1.82 g, 18.0 mmol, 3 eq.) and the mixture was stirred at room temperature for 15 mins. Boc-anhydride (2.64 g, 12.1 mmol, 2 eq.) was added and stirring continued at room temperature for 2 h. The resulting mixture was diluted with water (50 mL) and extracted with DCM (2×30 mL). The combined organic extracts were dried over Na₂SO₄, filtered and concentrated under vacuum. The crude material was purified by chromatography on silica (product eluted at 8% EtOAc in hexane) to yield the title compound as an off-white solid (1.6 g, Yield: 80.0%).

¹H NMR (DMSO-d6, 400 MHz): δ 1.45 (s, 9H), 3.75 (s, 3H), 4.43 (d, J=10.4 Hz, 2H), 4.64 (d, J=9.6 Hz, 2H), 6.95 (d, J=4.8 Hz, 1H), 7.08 (s, 1H).

LCMS (Method A): 2.509 min, MS: ES+ 271.9 (M−56).

### Step 2: tert-Butyl (R)-6-methoxy-4-((tetrahydrofuran-3-yl)amino)isoindoline-2-carboxylate

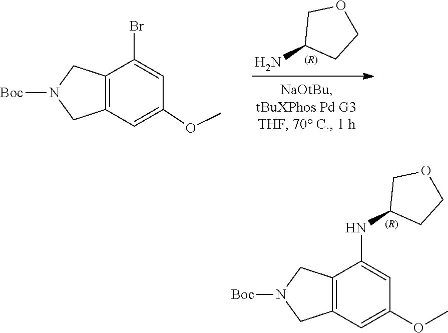

To a stirred solution of tert-butyl 4-bromo-6-methoxyisoindoline-2-carboxylate (step 1) (0.8 g, 2.44 mmol, 1 eq.) in THF (16 mL) were added (R)-tetrahydrofuran-3-amine (CAS 111769-26-7) (0.256 g, 2.93 mmol, 1.2 eq.) and NaOtBu (0.586 g, 6.1 mmol, 2.5 eq.) at room temperature. The mixture was degassed under N₂ gas for 15 min and treated with tBuXphos Pd G3 (0.116 g, 0.14 mmol, 0.06 eq.). The reaction mixture was heated to 70° C. and stirred for 1 h. The resulting mixture was diluted with water (50 mL) and extracted with EtOAc (2×30 mL). The combined organic extracts were dried over Na₂SO₄, filtered and concentrated under vacuum. The crude material was purified by chromatography on silica (product eluted at 22% EtOAc in hexane) to yield the title compound as a yellow solid (0.650 g, Yield: 80.0%).

¹H NMR (DMSO-d6, 400 MHz): δ 1.46 (s, 9H), 1.80-1.85 (m, 1H), 2.13-2.18 (m, 1H), 3.68-3.73 (m, 4H), 3.78-4.00 (m, 3H), 4.33 (d, J=5.2 Hz, 2H), 4.45 (d, J=11.2 Hz, 2H), 5.41 (q, J=6.0, 11.2 Hz, 1H), 5.96 (bs, 1H), 6.15 (d, J=6.8 Hz, 1H).

LCMS (Method A): 2.040 min, MS: ES+ 335 (M+1).

### Step 3: (R)-6-Methoxy-N-(tetrahydrofuran-3-yl)isoindolin-4-amine hydrochloride

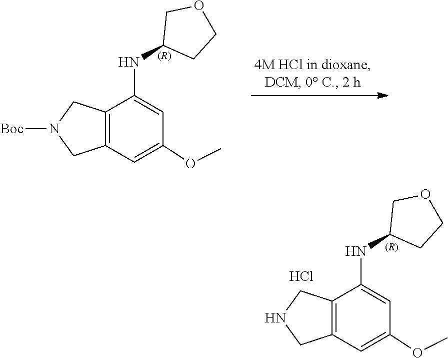

To a stirred solution of tert-butyl (R)-6-methoxy-4-((tetrahydrofuran-3-yl)amino)isoindoline-2-carboxylate (step 2) (0.650 g, 1.94 mmol, 1 eq.) in DCM (13 mL) cooled to 0° C. was added dropwise 4M HCl in dioxane (6.5 mL) and the mixture was stirred at 0° C. for 2 h. The resulting mixture was concentrated under vacuum and the crude material was triturated with diethyl ether (3×10 mL) and dried under high vacuum to yield the title compound as a brown solid (0.5 g, Yield: 95.0%).

¹H NMR (DMSO-d6, 400 MHz): δ 1.77-1.85 (m, 1H), 2.14-2.23 (m, 1H), 3.55-3.58 (m, 3H), 3.70-3.72 (m, 4H), 4.00-4.05 (m, 2H), 4.24 (t, J=4.8 Hz, 2H), 4.34 (t, J=5.2 Hz, 2H), 6.03 (d, J=1.6 Hz, 1H), 6.23 (s, 1H), 9.80 (s, 2H).

LCMS (Method A): 0.885 min, MS: ES+ 235.1 (M+1).

### Intermediate GA (S)-6-Methoxy-N-(tetrahydrofuran-3-yl)isoindolin-4-amine hydrochloride

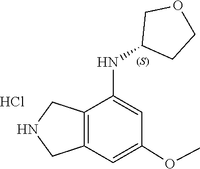

The title compound was prepared from tert-butyl 4-bromo-6-methoxyisoindoline-2-carboxylate (Intermediate G step 1) and (S)-tetrahydrofuran-3-amine analogously to Intermediate G steps 2 and 3.

¹H NMR (DMSO-d6, 400 MHz): δ 1.79-1.85 (m, 1H), 2.16-2.21 (m, 1H), 3.53-3.73 (m, 5H), 3.87-4.04 (m, 4H), 4.24 (t, J=4.8 Hz, 2H), 4.34 (t, J=5.2 Hz, 2H), 6.03 (d, J=1.6 Hz, 1H), 6.23 (s, 1H), 9.78 (s, 2H).

LCMS (Method A): 0.884 min, MS: ES+ 235.1 (M+1).

### Intermediate H (R)-6-(2-(Dimethylamino)ethoxy)-N-(tetrahydrofuran-3-yl)isoindolin-4-amine hydrochloride

### Step 1: 7-Bromoisoindolin-5-ol hydrochloride

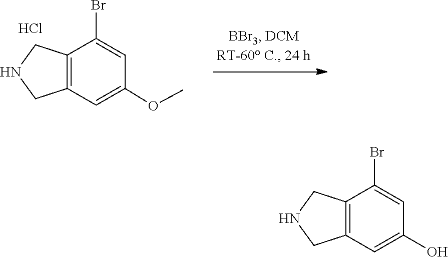

BBr₃ (1M in DCM) (40 mL) was added dropwise to 4-bromo-6-methoxyisoindoline hydrochloride (CAS142740090-5 free base) (2.0 g, 7.6 mmol, 1 eq.) at room temperature. The resulting mixture was heated to 60° C. and stirred for 16 h. The resulting mixture was concentrated under vacuum and the crude material was diluted with water (50 mL) causing a solid to precipitate. The solid was collected by filtration and dried under high vacuum to yield the title compound as an off-white solid (1.35 g, Yield: 72.0%).

¹H NMR (DMSO-d6, 400 MHz): δ 3.94 (s, 2H), 4.24 (s, 2H), 6.27-6.31 (m, 1H), 6.42-6.46 (m, 1H).

LCMS (Method A): 0.688 min, MS: ES+ 213.9 & 215.9 (M+1 & M+3).

### Step 2: tert-Butyl 4-bromo-6-hydroxyisoindoline-2-carboxylate

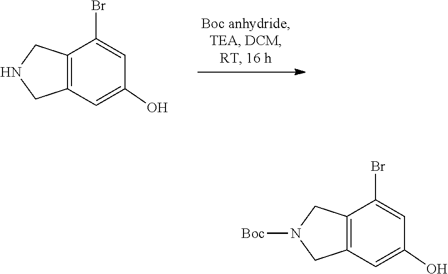 [SMILES: Oc1cc(Br)c2c(c1)CNC2 ; CC(C)(C)OC(=O)N1Cc2cc(O)cc(Br)c2C1]

To a stirred solution of 7-bromoisoindolin-5-ol hydrochloride (step 1) (1.3 g, 6.1 mmol, 1 eq.) in DCM (26 mL) was added TEA (1.85 g, 18.3 mmol, 3 eq.) and the mixture was stirred at room temperature for 15 mins. Boc-anhydride (1.33 g, 6.1 mmol, 1 eq.) was added and stirring continued at room temperature for 2 h. The resulting mixture was diluted with water (50 mL) and extracted with DCM (5×30 mL). The combined organic extracts were dried over Na₂SO₄, filtered and concentrated under vacuum. The crude material was purified by chromatography on silica (product eluted at 45% EtOAc in hexane) to yield the title compound as an off-white solid (1.150 g, Yield: 55.0%).

¹H NMR (DMSO-d6, 400 MHz): δ 1.45 (s, 9H), 4.39 (d, J=10.4 Hz, 2H), 4.59 (d, J=10.4 Hz, 2H), 6.73 (s, 1H), 6.87 (d, J=1.6 Hz, 1H), 9.91 (s, 1H).

LCMS (Method A): 2.024 min, MS: ES+ 257.8 (M−56).

### Step 3: 4-Bromo-6-(2-(dimethylamino)ethoxy)isoindoline-2-carboxylate

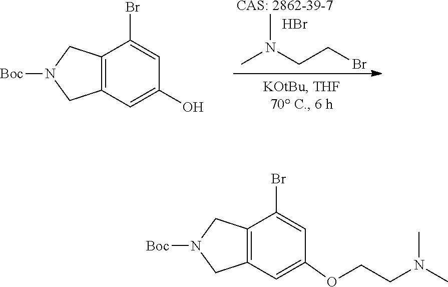

To a stirred solution of tert-butyl 4-bromo-6-hydroxyisoindoline-2-carboxylate (step 2) (0.2 g, 0.63 mmol, 1 eq.) in THF (10 mL) was added KOtBu (0.353 g, 3.15 mmol, 5 eq.) and the mixture was stirred at room temperature for 1 h. 2-Bromo-N,N-dimethylethan-1-amine hydrobromide (CAS 2862-39-7) (0.447 g, 1.91 mmol, 3 eq.) was added and the reaction mixture was heated to 70° C. for 6 h. The resulting mixture was diluted with water (100 mL) and extracted with EtOAc (4×30 mL). The combined organic extracts were dried over Na₂SO₄, filtered and concentrated under vacuum. The crude material was triturated with diethyl ether (2×15 mL) and dried under high vacuum to yield the title compound as a yellow solid (1.05 g, Yield: 63.0%).

¹H NMR (DMSO-d6, 400 MHz): δ 1.45 (s, 9H), 2.19 (s, 6H), 2.59 (d, J=5.6 Hz, 2H), 4.03-4.06 (m, 2H), 4.44 (d, J=10.4 Hz, 2H), 4.64 (d, J=6.0 Hz, 2H), 6.96 (d, J=6.8 Hz, 1H), 7.09 (bs, 1H).

LCMS (Method A): 1.461 min, MS: ES+ 384.9 & 386.9 (M+1 & M+3).

### Step 4: tert-Butyl (R)-6-(2-(dimethylamino)ethoxy)-4-((tetrahydrofuran-3-yl)amino)isoindoline-2-carboxylate

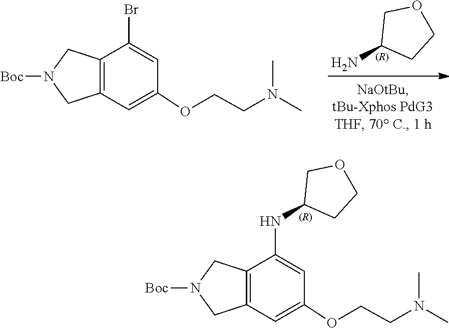

To a stirred solution of tert-butyl 4-bromo-6-(2-(dimethylamino)ethoxy)isoindoline-2-carboxylate (step 3) (0.5 g, 1.30 mmol, 1 eq.) in THF (10 mL) were added (R)-tetrahydrofuran-3-amine (CAS 111769-26-7) (0.136 g, 1.56 mmol, 1.2 eq.) and NaOtBu (0.312 g, 3.25 mmol, 2.5 eq.) and the mixture was degassed under N₂ gas at room temperature for 15 mins. tBuXphos PdG3 (0.062 g, 0.078 mmol, 0.06 eq.) was added and the mixture heated to 70° C. for 1 h. The resulting mixture was diluted with water (50 mL) and extracted with EtOAc (5×30 mL). The combined organic extracts were dried over Na₂SO₄, filtered and concentrated under vacuum. The crude material was purified by chromatography on silica (product eluted at 3.7% MeOH in DCM) to yield the title compound as a yellow solid (0.350 g, Yield: 69.0%).

¹H NMR (DMSO-d6, 400 MHz): δ 1.45 (d, J=5.2 Hz, 9H), 1.80-1.84 (m, 1H), 2.12-2.19 (m, 1H), 2.24 (s, 6H), 2.64 (t, J=5.2 Hz, 2H), 3.54-3.59 (m, 1H), 3.68-3.73 (m, 1H), 3.78-3.90 (m, 2H), 3.96-4.02 (m, 3H), 4.33 (d, J=5.6 Hz, 2H), 4.44 (d, J=11.6 Hz, 2H), 5.42 (q, J=6.4, 10.8 Hz, 1H), 5.96 (bs, 1H), 6.17 (d, J=9.2 Hz, 1H).

LCMS (Method A): 1.274 min, MS: ES+ 392.1 (M+1).

### Step 5: (R)-6-(2-(Dimethylamino)ethoxy)-N-(tetrahydrofuran-3-yl)isoindolin-4-amine hydrochloride

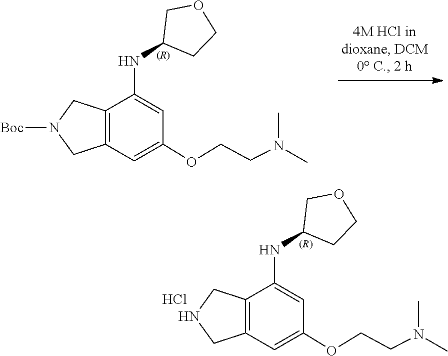

To a stirred solution of tert-butyl (R)-6-(2-(dimethylamino)ethoxy)-4-((tetrahydrofuran-3-yl)amino)isoindoline-2-carboxylate (step 4) (0.350 g, 0.89 mmol, 1 eq.) in DCM (4 mL) cooled to 0° C. was added dropwise 4M HCl in dioxane (1.7 mL) and the mixture was stirred at 0° C. for 2 h. The resulting mixture was concentrated under vacuum and the crude material was triturated with diethyl ether (3×10 mL). The resulting solid was dried under high vacuum to yield the title compound as a brown solid (0.35 g, Yield: Quantitative).

¹H NMR (DMSO-d6, 400 MHz): δ 1.82-1.85 (m, 1H), 2.17-2.22 (m, 1H), 2.81 (d, J=4.8 Hz, 6H) 3.46-3.48 (m, 2H), 3.56-3.59 (m, 2H), 3.69-3.75 (m, 1H), 3.81-3.92 (m, 2H), 4.00-4.05 (m, 1H) 4.20-4.35 (m, 6H), 6.11 (s, 1H), 6.28 (s, 1H), 6.23 (s, 1H), 9.94 (bs, 2H).

LCMS (Method A): 2.04 min, MS: ES+ 292.4 (M+1).

### Intermediate I (S)-6-(2-(Dimethylamino)ethoxy)-N-(tetrahydrofuran-3-yl)isoindolin-4-amine hydrochloride

### Step 1: (S)-6-(2-(Dimethylamino)ethoxy)-4-((tetrahydrofuran-3-yl)amino)isoindoline-2-carboxylate

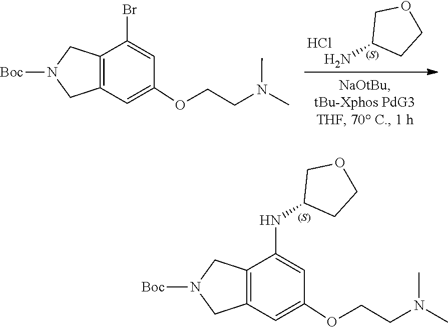

To a stirred solution of tert-butyl 4-bromo-6-(2-(dimethylamino)ethoxy)isoindoline-2-carboxylate (Intermediate H step 3) (0.5 g, 1.30 mmol, 1 eq.) in THF (10 mL) were added (S)-tetrahydrofuran-3-amine (CAS 204512-95-8) (0.192 g, 1.56 mmol, 1.2 eq.) and NaOtBu (0.312 g, 3.25 mmol, 2.5 eq.) at room temperature and the mixture was degassed under N₂ gas for 15 mins. tBuXphosPdG3 (0.062 g, 0.078 mmol, 0.06 eq.) was added and the reaction mixture was heated to 70° C. for 1 h. The resulting mixture was diluted with water (50 mL) and extracted with EtOAc (5×30 mL). The combined organic extracts were dried over Na₂SO₄, filtered and concentrated under vacuum. The crude material was purified by chromatography on silica (product eluted at 3.5% MeOH in DCM) to yield the title compound as a yellow solid (0.350 g, Yield: 69.0%).

¹H NMR (DMSO-d6, 400 MHz): δ 1.45 (d, J=5.6 Hz, 9H), 1.81-1.83 (m, 1H), 2.12-2.17 (m, 1H), 2.23 (s, 6H), 2.63-2.67 (m, 2H), 3.54-3.59 (m, 1H), 3.70-3.73 (m, 1H), 3.78-3.90 (m, 2H), 3.98-4.01 (m, 3H), 4.33 (d, J=5.6 Hz, 2H), 4.44 (d, J=11.6 Hz, 2H), 5.40-5.44 (m, 1H), 5.96 (bs, 1H), 6.16 (d, J=11.2 Hz, 1H).

LCMS (Method A): 1.279 min, MS: ES+ 392.1 (M+1).

### Step 2: (S)-6-(2-(Dimethylamino)ethoxy)-N-(tetrahydrofuran-3-yl)isoindolin-4-amine hydrochloride

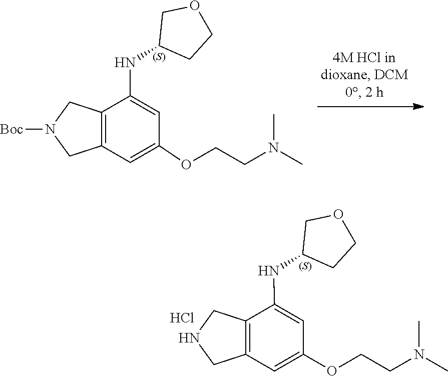

To a stirred solution of tert-butyl (S)-6-(2-(dimethylamino)ethoxy)-4-((tetrahydrofuran-3-yl)amino)isoindoline-2-carboxylate (step 1) (0.350 g, 0.89 mmol, 1 eq.) in DCM (4 mL) cooled to 0° C. was added dropwise 4M HCl in dioxane (1.7 mL). The reaction mixture was stirred at 0° C. for 2 h. The resulting mixture was concentrated under vacuum to afford a crude residue. The crude material was triturated with diethyl ether (3×10 mL) and dried under high vacuum to yield the title compound as a pink solid (0.35 g, Yield: Quantitative).

¹H NMR (DMSO-d6, 400 MHz): δ 1.82-1.84 (m, 1H), 2.17-2.20 (m, 1H), 2.82 (d, J=4.8 Hz, 6H) 3.47-3.58 (m, 5H), 3.69-3.75 (m, 2H), 4.00-4.02 (m, 2H), 4.25-4.36 (m, 5H), 6.11 (s, 1H), 6.28 (s, 1H), 9.85 (bs, 2H).

LCMS (Method A): 1.77 min, MS: ES+ 292.3 (M+1).

### Intermediate IA N-(Oxazol-4-ylmethyl)isoindolin-4-amine hydrochloride

### Step 1: tert-Butyl 4-((oxazol-4-ylmethyl)amino)isoindoline-2-carboxylate

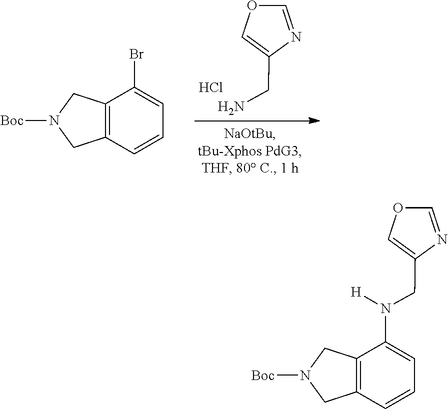 [SMILES: CC(C)(C)OC(=O)N1Cc2cccc(Br)c2C1 ; Cl ; NCc1cocn1 ; CC(C)(C)OC(=O)N1Cc2cccc(NCc3cocn3)c2C1]

The title compound was prepared from tert-butyl 4-bromoisoindoline-2-carboxylate (Intermediate N step 1) and oxazol-4-ylmethanamine hydrochloride (CAS 1072806-60-0) analogously to Intermediate I step 1.

¹H NMR (DMSO-d6, 400 MHz): δ 1.47 (d, J=5.6 Hz, 9H), 4.20-4.22 (m, 2H), 4.43 (d, J=7.6 Hz, 2H), 4.51 (d, J=10.8 Hz, 2H), 5.81-5.84 (m, 1H), 6.51 (d, J=8 Hz, 1H), 6.53 (t, J=5.6 Hz, 1H), 7.04 (t, J=7.6 Hz, 1H), 7.91 (d, J=3.2 Hz, 1H), 8.31 (s, 1H).

LCMS (Method A): 1.986 min, MS: ES+ 215.83 (M−100).

### Step 2: N-(Oxazol-4-ylmethyl)isoindolin-4-amine hydrochloride

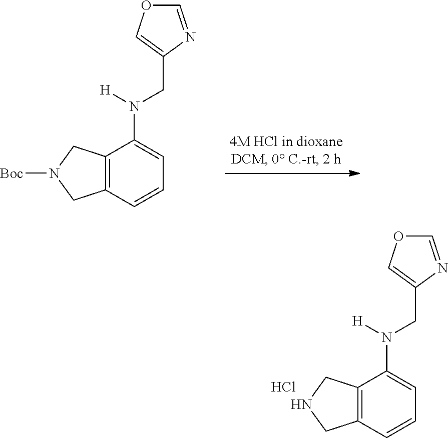 [SMILES: CC(C)(C)OC(=O)N1Cc2cccc(NCc3cocn3)c2C1 ; Cl ; c1cc2c(c(NCc3cocn3)c1)CNC2]

The title compound was prepared from tert-butyl 4-((oxazol-4-ylmethyl)amino)isoindoline-2-carboxylate (step 1) and 4M HCl in dioxane analogously to Intermediate I step 2.

¹H NMR (DMSO-d6, 400 MHz): 4.23 (s, 2H), 2.32 (t, J=5.2 Hz, 2H), 4.0 (t, J=5.2 Hz, 2H), 6.57-6.62 (m, 2H), 7.12 (t, J=7.6 Hz, 2H), 7.98 (s, 1H), 8.33 (s, 1H), 9.91 (s, 2H).

LCMS (Method A): 0.761 min, MS: ES+ 215.83 (M+1).

### Intermediate J 4-Cyano-2,6-bis(methoxymethoxy)benzoic acid

### Step 1: 4-Bromo-3,5-bis(methoxymethoxy)benzonitrile

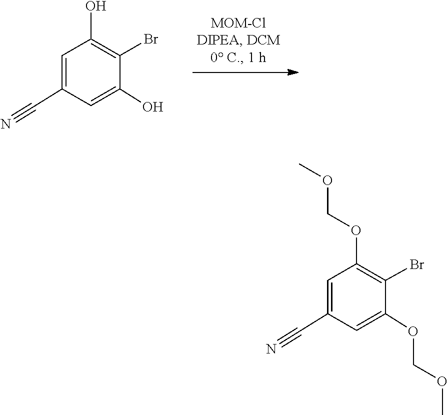 [SMILES: N#Cc1cc(O)c(Br)c(O)c1 ; COCOc1cc(C#N)cc(OCOC)c1Br]

To a stirred solution of 4-bromo-3,5-dihydroxybenzonitrile (CAS 2385736-09-2) (14 g, 65.40 mmol, 1 eq.) in DCM (200 mL) was added DIPEA (25.29 g, 196.2 mmol, 3.0 eq.) and the mixture was stirred at room temperature for 30 mins. The mixture was cooled to 0° C., treated dropwise with MOM-chloride (10.5 g, 130.8 mmol, 2.0 eq.) and stirred at 0° C. for 1 h. The resulting mixture was diluted with water (500 mL) and extracted with DCM (3×100 mL). The combined organic extracts were dried over Na₂SO₄, filtered and concentrated under vacuum. The crude material was purified by chromatography on silica (product eluted at 20% EtOAc in hexane) to yield the title compound as an off-white solid (10 g, Yield: 50.0%).

¹H NMR (DMSO-d6, 400 MHz): δ 3.41 (s, 6H), 5.37 (s, 4H), 7.34 (s, 2H).

### Step 2: 4-Formyl-3,5-bis(methoxymethoxy)benzonitrile

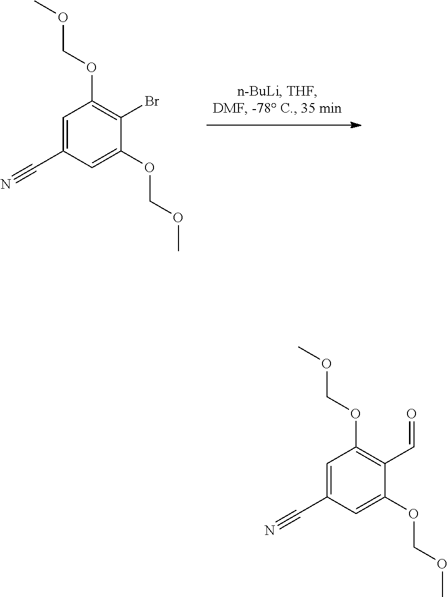 [SMILES: COCOc1cc(C#N)cc(OCOC)c1Br ; COCOc1cc(C#N)cc(OCOC)c1C=O]

A stirred solution of 4-bromo-3,5-bis(methoxymethoxy)benzonitrile (step 1) (3.0 g, 9.93 mmol, 1 eq.) in THF (30 mL) was cooled to −78° C. and treated dropwise with n-BuLi (1.6M in Hexane) (12.4 mL, 19.86 mmol, 2 eq.). After stirring at −78° C. for 30 min, DMF (6 mL, 2 eq.) was added in one portion and stirring continued at −78° C. for 5 mins. The reaction was quenched with saturated solution of NH₄Cl (500 mL) and the mixture was extracted with EtOAc (5×50 mL). The combined organic extracts were dried over Na₂SO₄, filtered and concentrated under vacuum to yield the title compound as a red liquid (15 g, Yield: quantitative). The crude material was used in the next step without further purification.

### Step 3: 4-Cyano-2,6-bis(methoxymethoxy)benzoic acid

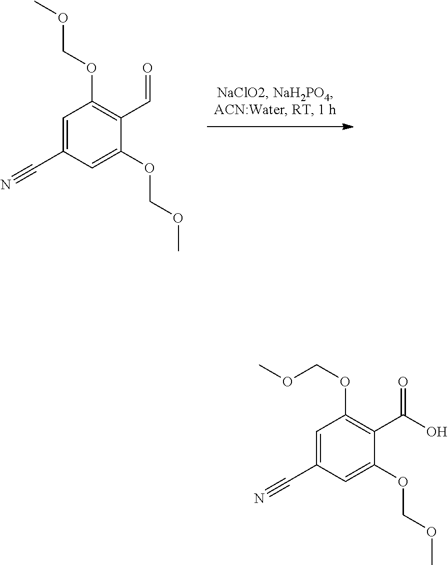 [SMILES: COCOc1cc(C#N)cc(OCOC)c1C=O ; COCOc1cc(C#N)cc(OCOC)c1C(=O)O]

To a stirred solution of 4-formyl-3,5-bis(methoxymethoxy)benzonitrile (step 2) (15 g, 59.7 mmol, 1 eq.) in MeCN (50 mL):water (50 mL) were added portionwise NaClO₂ (13.45 g, 149.4 mmol, 2.5 eq.) and NaH₂PO₄ (10.75 g, 89.5 mmol, 1.5 eq.) at 20° C. The reaction mixture was stirred at 20° C. for 1 h. The resulting mixture was diluted with water (50 mL) and extracted with DCM (3×30 mL). The aqueous phase was acidified with saturated KHSO₄ solution (30 mL) and extracted with 10% MeOH in DCM (5×50 mL). The combined organic extracts were dried over Na₂SO₄, filtered and concentrated under vacuum to yield the title compound as a red solid (4 g, Yield: 89.2%).

¹H NMR (DMSO-d6, 400 MHz): δ 3.44 (s, 6H) (merged in moisture peak), 5.30 (s, 4H), 7.32 (s, 2H), 13.39 (s, 1H).

LCMS (Method A): 1.260 min, MS: ES+ 266 (M−1).

### Intermediate K 2-(Benzyloxy)-4-cyano-6-hydroxybenzoic acid

### Step 1: 4-Cyano-2,6-dihydroxybenzoic acid

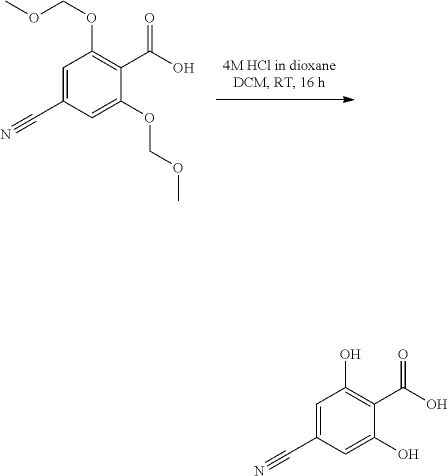 [SMILES: COCOc1cc(C#N)cc(OCOC)c1C(=O)O ; N#Cc1cc(O)c(C(=O)O)c(O)c1]

The title compound was prepared from 4-cyano-2,6-bis(methoxymethoxy)benzoic acid (Intermediate J) and 4M HCl in dioxane analogously to Example 12 step 3.

¹H NMR (DMSO-d6, 400 MHz): δ 3.57 (s, 2H), 6.50 (s, 1H).

LCMS (Method A): 1.171 min, MS: ES+ 177.9 (M−1).

### Step 2: 5-Hydroxy-2,2-dimethyl-4-oxo-4H-benzo[d][1,3]dioxine-7-carbonitrile

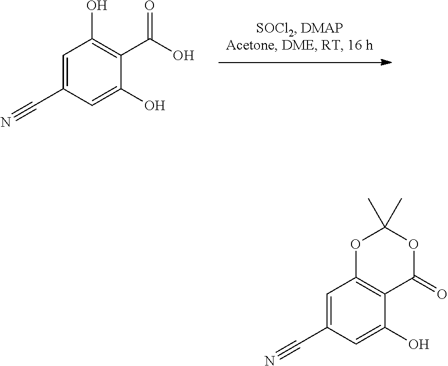 [SMILES: N#Cc1cc(O)c(C(=O)O)c(O)c1 ; CC1(C)OC(=O)c2c(O)cc(C#N)cc2O1]

The title compound was prepared from 4-cyano-2,6-dihydroxybenzoic acid (step 1) and SOCl₂ analogously to Intermediate F step 1.

¹H NMR (DMSO-d6, 400 MHz): δ 1.71 (s, 6H), 7.07-7.09 (m, 2H), 10.91 (s, 1H).

### Step 3: 5-(Benzyloxy)-2,2-dimethyl-4-oxo-4H-benzo[d][1,3]dioxine-7-carbonitrile

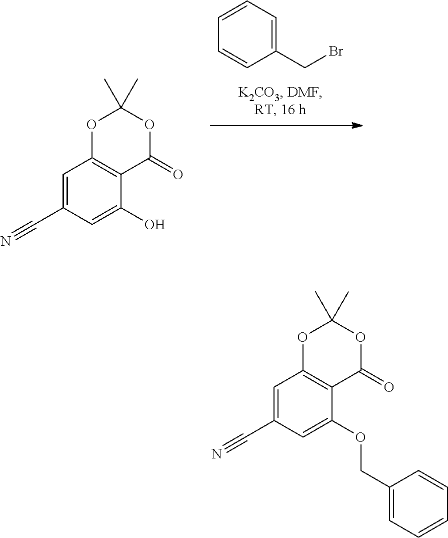 [SMILES: BrCc1ccccc1 ; CC1(C)OC(=O)c2c(O)cc(C#N)cc2O1 ; CC1(C)OC(=O)c2c(OCc3ccccc3)cc(C#N)cc2O1]

The title compound was prepared from 5-hydroxy-2,2-dimethyl-4-oxo-4H-benzo[d][1,3]dioxine-7-carbonitrile (step 2) and benzyl bromide analogously to Intermediate F step 2.

¹H NMR (DMSO-d6, 400 MHz): δ 1.69 (s, 6H), 5.33 (s, 2H), 7.27 (s, 1H), 7.35-7.36 (s, 1H), 7.40-7.44 (m, 2H), 7.48 (s, 1H), 7.53-7.55 (m, 2H).

### Step 4: 2-(Benzyloxy)-4-cyano-6-hydroxybenzoic acid

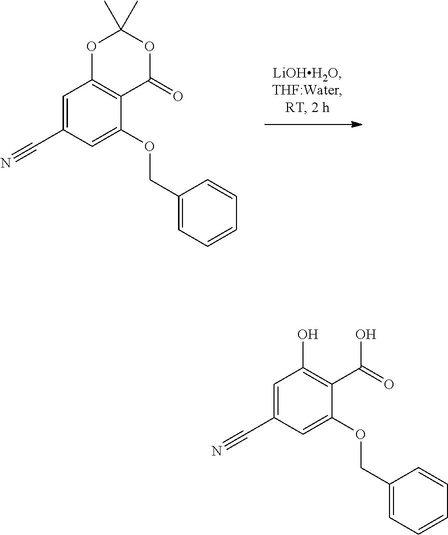 [SMILES: CC1(C)OC(=O)c2c(OCc3ccccc3)cc(C#N)cc2O1 ; N#Cc1cc(O)c(C(=O)O)c(OCc2ccccc2)c1]

The title compound was prepared from 5-(benzyloxy)-2,2-dimethyl-4-oxo-4H-benzo[d][1,3]dioxine-7-carbonitrile (step 3) and LiOH·H₂O analogously to Intermediate F step 3.

¹H NMR (DMSO-d6, 400 MHz): δ 5.18 (s, 2H), 6.86 (s, 1H), 7.11 (s, 1H), 7.32-7.41 (m, 5H), 10.88 (bs, 1H), 13.14 (bs, 1H).

### Intermediate KA: 4-Cyano-2-hydroxy-6-(pyridin-2-ylmethoxy)benzoic acid

### Step 1: 2,2-Dimethyl-4-oxo-5-(pyridin-2-ylmethoxy)-4H-benzo[d][1,3]dioxine-7-carbonitrile

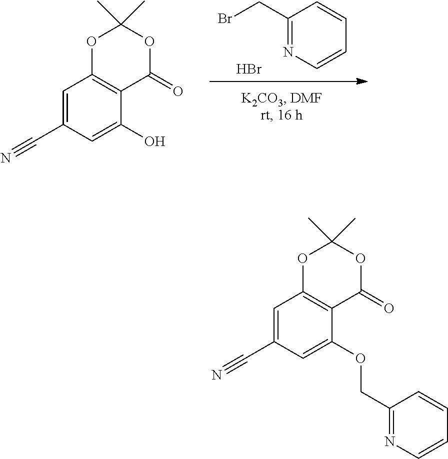 [SMILES: CC1(C)OC(=O)c2c(O)cc(C#N)cc2O1 ; BrCc1ccccn1 ; Br ; CC1(C)OC(=O)c2c(OCc3ccccn3)cc(C#N)cc2O1]

The title compound was prepared from 5-hydroxy-2,2-dimethyl-4-oxo-4H-benzo[d][1,3]dioxine-7-carbonitrile (Intermediate K step 2) and 2-(bromomethyl)pyridine hydrobromide analogously to Intermediate F step 2.

¹H NMR (DMSO-d6, 400 MHz): δ 1.70 (s, 6H), 5.37 (s, 2H), 7.30 (s, 1H), 7.37 (d, J=5.2 Hz, 1H), 7.53 (s, 1H), 7.82 (d, J=7.6 Hz, 1H), 7.90 (t, J=7.6 Hz, 1H), 8.59 (d, J=4.4 Hz, 1H).

LCMS (Method N): 1.509 min, MS: ES+ 310.9 (M−100).

### Step 2: 4-Cyano-2-hydroxy-6-(pyridin-2-ylmethoxy)benzoic acid

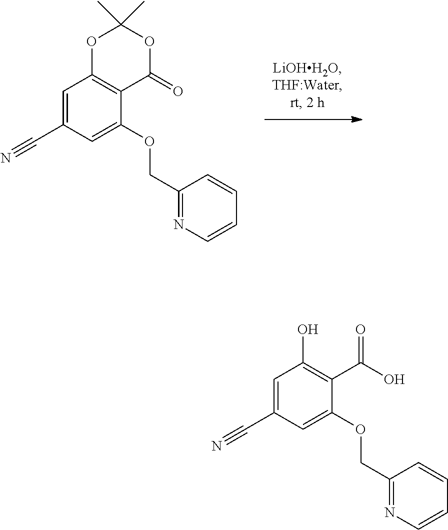 [SMILES: CC1(C)OC(=O)c2c(OCc3ccccn3)cc(C#N)cc2O1 ; N#Cc1cc(O)c(C(=O)O)c(OCc2ccccn2)c1]

The title compound was prepared from 2,2-dimethyl-4-oxo-5-(pyridin-2-ylmethoxy)-4H-benzo[d][1,3]dioxine-7-carbonitrile (step 1) and LiOH·H₂O analogously to Intermediate F step 3.

¹H NMR (DMSO-d6, 400 MHz): δ 5.25 (s, 2H), 6.87 (s, 1H), 7.10 (s, 1H), 7.36 (dd, J=5.6, 7.2 Hz, 1H), 7.51 (d, J=8.0 Hz, 1H), 7.84-7.88 (m, 1H), 8.58 (d, J=4.0 Hz, 1H), 11.22 (bs, 2H).

LCMS (Method A): 1.104 min, MS: ES+ 270.8 (M+100)

### Intermediate L: 4-(Isoindolin-4-ylamino)-1-methylpyrrolidin-2-one hydrochloride

The title compound was prepared from tert-butyl 4-bromoisoindoline-2-carboxylate (Intermediate N step 1) and 4-amino-1-methylpyrrolidin-2-one (CAS: 933744-16-2) analogously to Intermediate H steps 4 and 5.

¹H NMR (DMSO-d6, 400 MHz): δ 2.22-2.27 (m, 1H), 2.67-2.76 (m, 3H), 3.19-3.22 (m, 1H), 3.57 (s, 1H), 3.72-3.76 (m, 1H), 4.12-4.16 (m, 1H), 4.30-4.42 (m, 5H), 6.52 (d, J=8.0, Hz, 1H), 6.65 (d, J=7.6 Hz, 1H), 7.17 (t, J=7.6 Hz, 1H), 9.84-9.87 (m, 1H).

LCMS (Method A): 0.747 min, MS: ES+ 231.9 (M+1).

### Intermediate LA: (S)-4-(isoindolin-4-ylamino)-1-methylpyrrolidin-2-one or (R)-4-(isoindolin-4-ylamino)-1-methylpyrrolidin-2-one and Intermediate LB: (S)-4-(isoindolin-4-ylamino)-1-methylpyrrolidin-2-one or (R)-4-(isoindolin-4-ylamino)-1-methylpyrrolidin-2-one

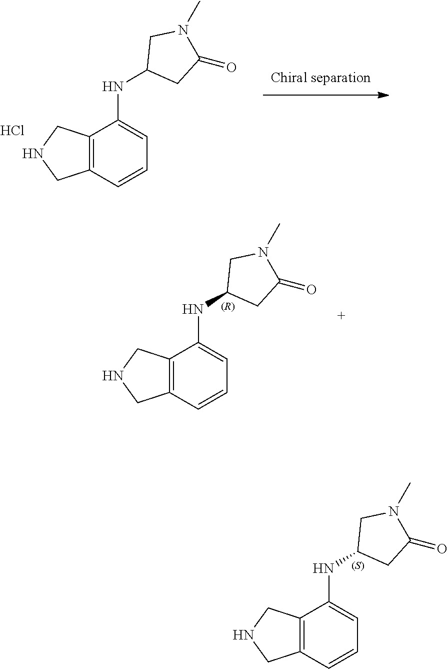

Chiral separation of 4-(Isoindolin-4-ylamino)-1-methylpyrrolidin-2-one hydrochloride (Intermediate L) under the following conditions afforded Intermediate LA and Intermediate LB:

Waters Prep SFC 350 with UV Detector. The column used was CHI RALPAK IA 250×4.6 mm Sum and the compounds were eluted with, Mobile Phase A: Liquid 002, Mobile Phase B: 0.1% Methanolic ammonia in Methanol:Acetonitrile (50:50) with an Isocratic Method of 50:50, Flow rate=4.0 ml/min; analysis time 8 mins.

### Intermediate LA: (S)-4-(Isoindolin-4-ylamino)-1-methylpyrrolidin-2-one or (R)-4-(isoindolin-4-ylamino)-1-methylpyrrolidin-2-one

¹H NMR (Fr-1) (DMSO-d6, 400 MHz): δ 2.22-2.27 (m, 1H), 2.67-2.75 (m, 4H), 3.17-3.22 (m, 2H), 3.73 (dd, J=6.8, 10 Hz, 1H), 4.13 (bs, 1H), 4.31 (s, 2H), 4.40 (s, 2H), 5.84 (d, J=6.4 Hz, 1H), 6.51 (d, J=8.0 Hz, 1H), 6.64 (d, J=7.6 Hz, 1H), 7.16 (t, J=8.0 Hz, 1H), 9.78 (bs, 1H).

SFC Retention Time=2.29 min

LCMS (Method A): 0.738 min, MS: ES+ 231.9 (M+1).

### Intermediate LB: (S)-4-(Isoindolin-4-ylamino)-1-methylpyrrolidin-2-one or (R)-4-(isoindolin-4-ylamino)-1-methylpyrrolidin-2-one

¹H NMR (Fr-2) (DMSO-d6, 400 MHz): δ 2.25 (dd, J=4.4, 16.8 Hz, 1H), 2.67 (t, J=8.8 Hz, 1H), 3.18 (dd, J=3.6, 9.6 Hz, 1H), 3.72 (dd, J=6.8, 9.6 Hz, 1H), 3.91-4.00 (m, 4H), 4.10-4.12 (m, 1H), 5.29 (d, J=6.8 Hz, 1H), 6.35 (d, J=8.0 Hz, 1H), 6.54 (d, J=7.2 Hz, 1H), 7.00 (t, J=7.6 Hz, 1H).

SFC Retention Time=3.92 min

LCMS (Method A): 0.741 min, MS: ES+ 231.8 (M+1).

### Intermediate M: (R)-6-(2-(Dimethylamino)ethoxy)-N-(tetrahydrofuran-3-yl)-1,2,3,4-tetrahydroisoquinolin-8-amine hydrochloride

### Step 1: tert-Butyl 8-bromo-6-(2-(dimethylamino)ethoxy)-3,4-dihydroisoquinoline-2(1H)-carboxylate

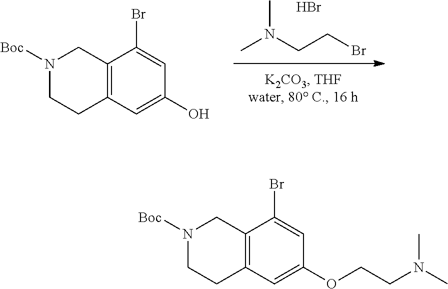

To a stirred solution of tert-butyl 8-bromo-6-hydroxy-3,4-dihydroisoquinoline-2(1H)-carboxylate (CAS 1579518-76-5) (7 g, 21.34 mmol, 1.0 eq.) in water:THF (2:1) (70 mL) was added K₂CO₃ (14.72 g, 106.70 mmol, 5.0 eq.) and the mixture was stirred at room temperature for 15 mins. 2-Bromo-N,N-dimethylethan-1-amine hydrobromide (CAS 2862-39-7) (7.4 g, 32.0 mmol, 1.5 eq.) and KI (0.35 g, 2.13 mmol, 0.1 eq.) were added and the mixture was heated to 80° C. and stirred for 16 h. The reaction mixture was poured into water (200 mL) and extracted in EtOAc (3×100 mL). The combined organic extracts were dried over Na₂SO₄, filtered and concentrated under vacuum. The crude material was purified by chromatography on silica (product eluted at 4% MeOH in DCM) to yield the title compound as a white solid (2 g, Yield: 22.5%).

¹H NMR (DMSO-d6, 400 MHz): δ 1.43 (s, 9H), 2.22 (s, 6H), 2.60-2.63 (m, 2H), 2.74-2.77 (m, 2H), 3.52 (m, 2H), 4.05 (t, J=5.6 Hz, 2H), 4.36 (s, 2H), 6.83 (s, 1H), 7.10 (s, 1H).

LCMS (Method A): LCMS (Method A): 1.489 min, MS: ES+ 400.76 (M+1)

### Step 2: tert-Butyl (R)-6-(2-(dimethylamino)ethoxy)-8-((tetrahydrofuran-3-yl)amino)-3,4-dihydroisoquinoline-2(1H)-carboxylate

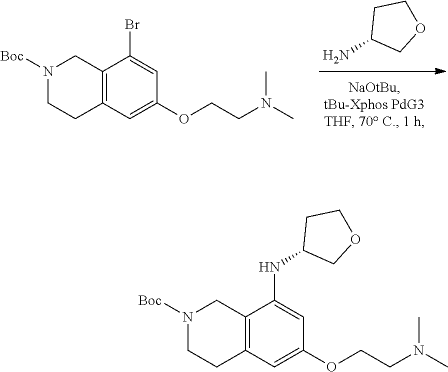 [SMILES: CN(C)CCOc1cc(Br)c2c(c1)CCN(C(=O)OC(C)(C)C)C2 ; N[C@@H]1CCOC1 ; CN(C)CCOc1cc2c(c(N[C@@H]3CCOC3)c1)CN(C(=O)OC(C)(C)C)CC2]

A stirred solution of tert-butyl 8-bromo-6-(2-(dimethylamino)ethoxy)-3,4-dihydroisoquinoline-2(1H)-carboxylate (step 1) (1 g, 2.5 mmol, 1 eq.), (R)-tetrahydrofuran-3-amine (0.327 g, 3.76 mmol, 1.5 eq.) and NaOtBu (0.72 g, 7.52 mmol, 3 eq.) in THF (10 mL) was degassed under N₂ gas at room temperature for 30 mins. tBuXphosPdG3 (0.119 g, 0.15 mmol, 0.06 eq.) was added and the reaction mixture was heated to 80° C. for 1 h. The resulting mixture was concentrated under vacuum and purification of the crude material by chromatography on silica (product eluted at 4% MeOH in DCM) yielded the title compound as sticky solid (0.2 g, Yield: 19%).

¹H NMR (DMSO-d6, 400 MHz): δ 1.43 (s, 9H), 1.85-1.86 (m, 1H), 2.12 (s, 6H), 2.60-2.65 (m, 4H), 3.49-3.53 (m, 2H), 3.58-3.61 (m, 1H), 3.71-3.75 (m, 1H), 3.80-3.84 (m, 1H), 3.89-3.93 (m, 1H), 3.96-3.98 (m, 3H), 4.20 (s, 2H), 4.87 (s, 1H), 5.99-5.06 (m, 1H).

LCMS (Method A): 1.341 min, MS: ES+ 406.01 (M+1).

### Step 3: (R)-6-(2-(Dimethylamino)ethoxy)-N-(tetrahydrofuran-3-yl)-1,2,3,4-tetrahydroisoquinolin-8-amine hydrochloride

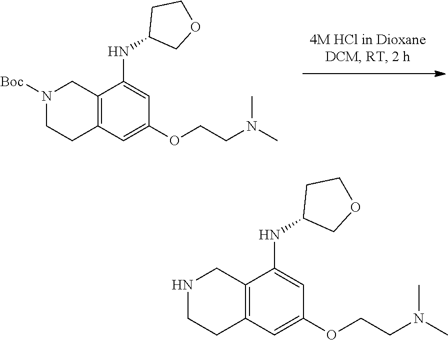 [SMILES: CN(C)CCOc1cc2c(c(N[C@@H]3CCOC3)c1)CN(C(=O)OC(C)(C)C)CC2 ; CN(C)CCOc1cc2c(c(N[C@@H]3CCOC3)c1)CNCC2]

A stirred solution of tert-butyl (R)-6-(2-(dimethylamino)ethoxy)-8-((tetrahydrofuran-3-yl)amino)-3,4-dihydroisoquinoline-2(1H)-carboxylate (0.2 g, 0.49 mmol, 1.0 eq.) in DCM (2 mL) cooled to 0° C. was treated dropwise with 4M HCl in dioxane (1 mL). The reaction mixture was allowed to warm to room temperature and stirred for 2 h. The resulting mixture was concentrated under vacuum and the crude material was triturated with diethyl ether (3×10 mL) followed by n-pentane (3×10 mL). The solid was dried under high vacuum to yield the title compound as brown solid (0.3 g, Quantitative, 0.98 mmol).

¹H NMR (DMSO-d6, 400 MHz): δ 1.60 (m, 1H), 1.85-1.88 (m, 1H), 2.17-2.25 (m, 1H), 2.81 (d, J=4.8 Hz, 6H), 2.89-2.92 (m, 2H), 3.25 (s, 2H), 3.46 (d, J=4.8 Hz, 2H), 3.70-3.76 (m, 1H), 3.81-3.87 (m, 3H), 3.91-3.95 (m, 1H), 4.31-4.33 (m, 2H), 6.13-6.19 (m, 1H), 9.74 (s, 2H), 10.75 (bs, 1H).

LCMS (Method A): 0.470 min, MS: ES+ 305.89 (M+1).

### Intermediate MA: (S)-6-(2-(Dimethylamino)ethoxy)-N-(tetrahydrofuran-3-yl)-1,2,3,4-tetrahydroisoquinolin-8-amine hydrochloride

### Step 1: (S)-6-(2-(dimethylamino)ethoxy)-8-((tetrahydrofuran-3-yl)amino)-3,4-dihydroisoquinoline-2(1H)-carboxylate

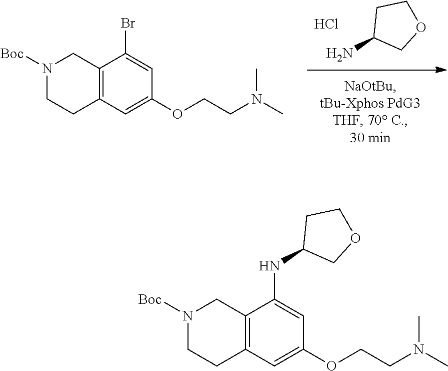 [SMILES: CN(C)CCOc1cc(Br)c2c(c1)CCN(C(=O)OC(C)(C)C)C2 ; Cl ; N[C@H]1CCOC1 ; CN(C)CCOc1cc2c(c(N[C@H]3CCOC3)c1)CN(C(=O)OC(C)(C)C)CC2]

A stirred solution of tert-butyl 8-bromo-6-(2-(dimethylamino)ethoxy)-3,4-dihydroisoquinoline-2(1H)-carboxylate (Intermediate M step 1) (1 g, 2.5 mmol, 1 eq.), (S)-tetrahydrofuran-3-amine (0.462 g, 3.76 mmol, 1.5 eq.) and NaOtBu (0.72 g, 7.52 mmol, 3 eq.) in THF (10 mL) was degassed under N₂ gas at room temperature for 30 mins. tBuXphosPdG3 (0.119 g, 0.15 mmol, 0.06 eq.) was added and the reaction mixture was heated to 80° C. for 1 h. The resulting mixture was concentrated under vacuum and purification of the crude material by chromatography on silica (product eluted at 4% MeOH in DCM) yielded the title compound as yellow liquid. (0.27 g, Yield: 26.6%).

¹H NMR (DMSO-d6, 400 MHz): δ 0.86 (s, 1H), 1.43 (s, 9H), 2.23-2.50 (m, 6H), 2.61-2.68 (m, 3H), 3.49-3.58 (m, 2H), 3.59-4.02 (m, 3H), 4.20-4.25 (m, 1H), 4.90 (bs, 1H), 6.49 (d, 1H), 6.21-6.25 (m, 1H).

LCMS (Method A): 1.320 min, MS: ES+ 406.01 (M+1)

### Step 2: (S)-6-(2-(Dimethylamino)ethoxy)-N-(tetrahydrofuran-3-yl)-1,2,3,4-tetrahydroisoquinolin-8-amine hydrochloride

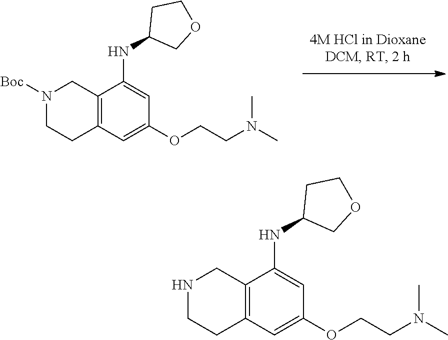 [SMILES: CN(C)CCOc1cc2c(c(N[C@H]3CCOC3)c1)CN(C(=O)OC(C)(C)C)CC2 ; CN(C)CCOc1cc2c(c(N[C@H]3CCOC3)c1)CNCC2]

A stirred solution of tert-butyl (S)-6-(2-(dimethylamino)ethoxy)-8-((tetrahydrofuran-3-yl)amino)-3,4-dihydroisoquinoline-2(1H)-carboxylate (step 1) (0.27 g, 0.67 mmol, 1.0 eq.) in DCM (2.7 mL) cooled to 0° C. was treated dropwise with 4M HCl in dioxane (1.3 mL). The reaction mixture was allowed to warm to room temperature and stirred for 2 h. The resulting mixture was concentrated under vacuum and the crude material was triturated with diethyl ether (3×10 mL) followed by n-pentane (3×10 mL). The resulting solid was dried under high vacuum to yield the title compound as brown solid (0.35 g, Quantitative, 1.15 mmol).

LCMS (Method A): 0.379 min, MS: ES+ 305.89 (M+1).

### Intermediate N: 1-(3-(Isoindolin-4-ylamino)azetidin-1-yl)ethan-1-one TFA salt

### Step 1: tert-4-bromoisoindoline-2-carboxylate

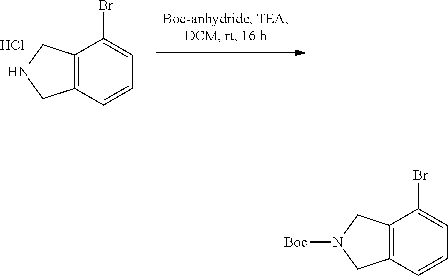 [SMILES: Cl ; Brc1cccc2c1CNC2 ; CC(C)(C)OC(=O)N1Cc2cccc(Br)c2C1]

To a mixture of 4-bromoisoindoline hydrochloride (CAS 923590-95-8) (15.0 g, 63.9 mmol, 1 eq.) in DCM (150 mL) was added TEA (26.7 mL, 191.8 mmol, 3 eq.) and the mixture was stirred at room temperature for 15 mins. The mixture was cooled to 0° C. and treated dropwise with Boc anhydride (29.4 mL, 127.8 mmol, 2 eq.). The reaction mixture was stirred at room temperature for 16 h. The resulting mixture was diluted with water (200 mL) and extracted with DCM (3×50 mL). The combined organic extracts were dried over Na₂SO₄, filtered and concentrated under vacuum. The crude material was purified by chromatography on silica (product eluted at 20% EtOAc in hexane) to yield the title compound (17 g, Yield: 87%).

¹H NMR (DMSO-d6, 400 MHz): δ 1.46 (d, J=2 Hz, 9H), 4.51-4.54 (m, 2H), 4.68-4.70 (m, 2H), 7.24-7.28 (m, 1H), 7.34-7.37 (m, 1H), 7.49 (d, J=7.6 Hz, 1H).

LCMS (Method A): 2.441 min, MS: ES+ 243.7 (M−56).

### Step 2: tert-Butyl 4-((1-acetylazetidin-3-yl)amino)isoindoline-2-carboxylate

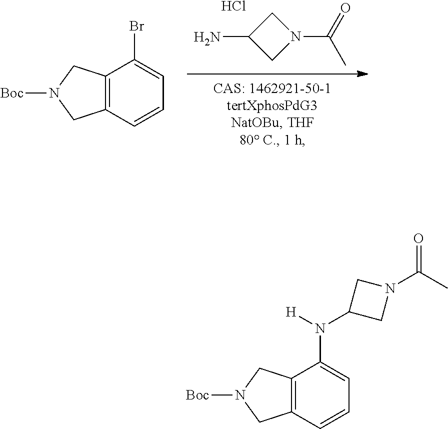

To a stirred solution of tert-butyl 4-bromoisoindoline-2-carboxylate (step 1)(0.6 g, 2.01 mmol, 1 eq.) in THF (6 mL) were added 1-(3-aminoazetidin-1-yl)ethan-1-one hydrochloride (CAS 1462921-50-1) (0.454 g, 3.01 mmol, 1.5 eq.), NaOtBu (0.58 g, 6.03 mmol, 3.0 eq.) and the reaction mixture was degassed under N₂ gas at room temperature for 20 mins. tBuXPhosPdG3 (0.096 g, 0.12 mmol, 0.06 eq.) was added and the reaction mixture was heated to 80° C. for 1 h. The resulting mixture was filtered through Celite® (filter material) washing through with DCM (200 mL). The combined organic extracts were dried over Na₂SO₄, filtered and concentrated under vacuum. The crude material was purified by chromatography on silica (product eluted at 4% MeOH in DCM) to yield the title compound as an off-white solid (0.8 g, Yield: 60.0%).

¹H NMR (DMSO-d6, 400 MHz): δ 1.45 (s, 9H), 1.99 (s, 3H), 3.71-3.74 (m, 1H), 3.89-3.92 (m, 1H), 4.13-4.21 (m, 2H), 4.42-4.53 (m, 4H), 5.89-5.94 (q, J=6 Hz, 12.8 Hz, 1H), 6.26-6.29 (q, J=4.4 Hz, 7.6 Hz, 1H), 6.61 (t, J=7.2 Hz, 1H), 7.08 (t, J=7.6 Hz, 1H).

LCMS (Method A): 1.798 min, MS: ES+ 232.2 (M−100).

### Step 3: 1-(3-(Isoindolin-4-ylamino)azetidin-1-yl)ethan-1-one TFA salt

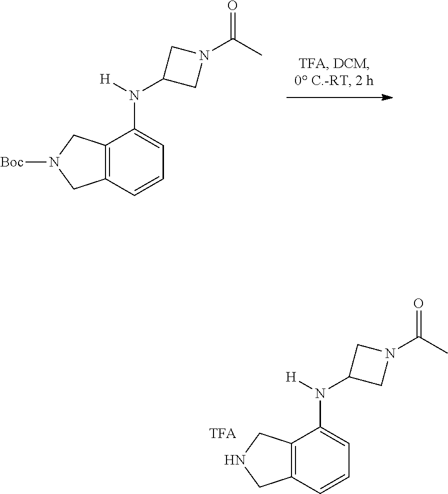 [SMILES: CC(=O)N1CC(Nc2cccc3c2CN(C(=O)OC(C)(C)C)C3)C1 ; CC(=O)N1CC(Nc2cccc3c2CNC3)C1]

To a stirred solution of tert-butyl 4-((1-acetylazetidin-3-yl)amino)isoindoline-2-carboxylate (step 2) (0.4 g, 1.20 mmol, 1.0 eq.) in DCM (4 mL) cooled to 0° C. was added dropwise TFA (2 mL) and the reaction mixture was stirred at room temperature for 2 h. The resulting mixture was concentrated under vacuum and the crude material was triturated with diethyl ether (2×5 mL). The solid material was dried under high vacuum to yield the title compound as a grey solid (0.38 g, Quantitative).

¹H NMR (DMSO-d6, 400 MHz): δ 1.77 (s, 3H), 3.85-3.89 (m, 2H), 4.17-4.23 (m, 2H), 4.34 (t, J=5.2 Hz, 2H), 4.43-4.50 (m, 2H), 6.36 (d, J=8 Hz, 1H), 6.69 (d, J=7.6 Hz, 1H), 7.18 (t, J=8 Hz, 1H), 9.33 (s, 1H).

LCMS (Method A): 0.738 min, MS: ES+ 232.1 (M+1).

### Intermediate NA N-(1-Methylazetidin-3-yl) isoindolin-4-amine TFA salt

### Step 1: tert-Butyl 4-((1-methylazetidin-3-yl) amino) isoindoline-2-carboxylate

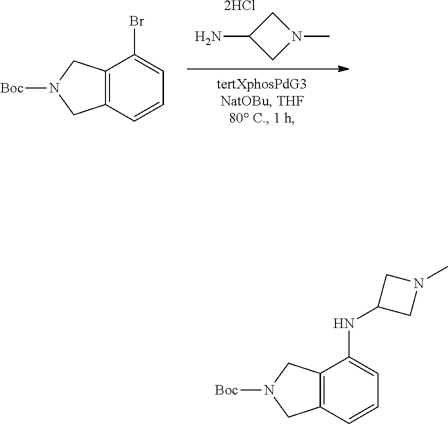

The title compound was prepared from tert-butyl 4-bromoisoindoline-2-carboxylate (Intermediate N step 1) and 3-amino-1-N-methyl-azetidine dihydrochloride (CAS 1139634-75-5) analogously to Intermediate N step 2.

¹H NMR (DMSO-d6, 400 MHz): δ 1.46-1.48 (m, 9H), 2.67 (s, 3H), 3.60-3.78 (m, 2H), 4.11-4.12 (m, 1H), 4.26-4.30 (m, 2H), 4.50-4.48 (m, 2H), 4.52-4.54 (m, 2H), 5.91-5.99 (m, 1H), 6.33 (d, J=8 Hz, 1H), 6.64 (t, J=7.6 Hz, 1H), 7.10 (t, J=7.6 Hz, 1H).

LCMS (Method A): 1.388 min, MS: ES+ 303.9 (M+1).

### Step 2: N-(1-Methylazetidin-3-yl) isoindolin-4-amine TFA salt

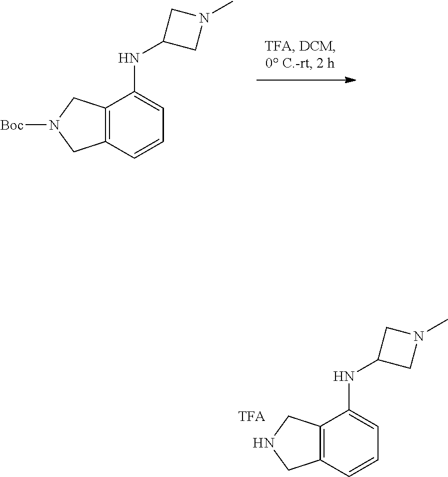 [SMILES: CN1CC(Nc2cccc3c2CN(C(=O)OC(C)(C)C)C3)C1 ; CN1CC(Nc2cccc3c2CNC3)C1]

The title compound was prepared from tert-butyl (S)-8-((tetrahydrofuran-3-yl)amino)-3,4-dihydroisoquinoline-2(1H)-carboxylate and TFA (1.75 mL) analogously to Intermediate N step 3.

¹H NMR (DMSO-d6, 400 MHz): δ 2.73-2.93 (m, 3H), 3.84-3.90 (m, 1H), 4.12-4.25 (m, 4H), 4.32 (t, J=16 Hz, 2H), 4.52-4.63 (m, 2H), 6.37 (dd, J=8 Hz, 1H), 6.72 (d, J=7.6 Hz, 1H), 7.18 (t, J=7.6 Hz, 1H), 9.63 (s, 2H).

LCMS (Method A): 0.197 min, MS: ES+ 203.9 (M+1).

### Intermediate NB: N-(Oxetan-3-yl)isoindolin-4-amine TFA salt

### Step 1: tert-Butyl 4-(oxetan-3-ylamino)isoindoline-2-carboxylate

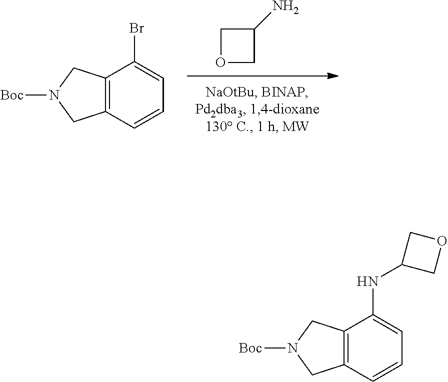 [SMILES: CC(C)(C)OC(=O)N1Cc2cccc(Br)c2C1 ; NC1COC1 ; CC(C)(C)OC(=O)N1Cc2cccc(NC3COC3)c2C1]

To a stirred solution of tert-butyl 4-bromoisoindoline-2-carboxylate (Intermediate N step 1) (1 g, 3.35 mmol, 1 eq) in 1,4-dioxane (10 mL) were added NaOtBu (0.450 g, 4.69 mmol, 1.4 eq) and oxetan-3-amine (CAS 21635-88-1) (0.490 g, 7.70 mmol, 2 eq) at room temperature. The mixture was degassed with nitrogen for 15 mins then treated with BINAP (0.125 g, 0.20 mmol, 0.06 eq) and Pd₂(dba)₃ (0.123 g, 0.13 mmol, 0.04 eq). The reaction mixture was heated to 130° C. using microwave radiation for 1.5 h. The resulting mixture was filtered through Celite® and washed with EtOAc (50 mL). The filtrate was concentrated under vacuum and purification of the crude material by flash column chromatography (eluting with 20% EtOAc in hexane) yielded the title compound as a light yellow solid (0.7 g, Yield: 71.88%).

¹H NMR (DMSO-d6, 400 MHz): δ 1.457 (d, J=7.2 Hz, 9H), 4.44-4.58 (m, 7H), 4.83 (t, J=6.0 Hz, 2H), 6.00 (t, J=6.4 Hz, 1H), 6.17 (dd, J=2.0, 8.0 Hz, 1H), 6.59 (t, J=7.2 Hz, 1H), 7.05 (t, J=7.6 Hz, 1H).

LCMS (Method N): 1.760 min, MS: ES+ 190.8 (M−100)

### Step 2: N-(Oxetan-3-yl)isoindolin-4-amine TFA salt

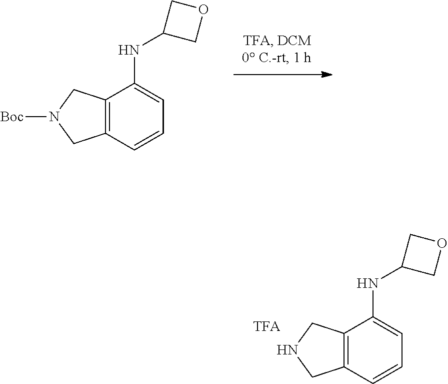 [SMILES: CC(C)(C)OC(=O)N1Cc2cccc(NC3COC3)c2C1 ; c1cc2c(c(NC3COC3)c1)CNC2]

The title compound was prepared from tert-butyl 4-(oxetan-3-ylamino)isoindoline-2-carboxylate (step 1) and TFA analogously to Intermediate N step 3.

¹H NMR (DMSO-d6, 400 MHz): δ 1.27-1.37 (m, 2H), 4.43-4.47 (m, 4H), 4.51-4.57 (m, 2H), 4.87 (t, J=6.4 Hz, 1H), 6.27 (d, J=8.0 Hz, 1H), 6.57-6.68 (m, 2H), 7.14 (t, J=8.0 Hz, 1H), 9.32 (bs, 2H).

LCMS (Method A): 0.895 min, MS: ES+ 190.9 (M+1).

### Intermediate NC: N-(Oxetan-3-yl)-1,2,3,4-tetrahydroisoquinolin-8-amine TFA salt

### Step 1: tert-Butyl 8-(oxetan-3-ylamino)-3,4-dihydroisoquinoline-2(1H)-carboxylate

The title compound was prepared from tert-butyl 8-bromo-3,4-dihydroisoquinoline-2(1H)-carboxylate and oxetan-3-amine analogously to Intermediate NB step 1.

¹H NMR (DMSO-d6, 400 MHz): δ 1.40 (s, 9H), 2.68 (t, J=5.6 Hz, 2H), 3.52 (t, J=5.6 Hz, 2H), 4.32 (s, 2H), 4.50 (d, J=2 Hz, 3H), 4.84 (s, 2H), 5.58 (bs, 1H), 6.10 (d, J=7.6 Hz, 1H), 6.48 (d, J=7.2 Hz, 1H), 6.96 (t, J=8 Hz, 1H).

LCMS (Method A): 1.97 min, MS: ES+ 248.9 (M−56).

### Step 2: N-(Oxetan-3-yl)-1,2,3,4-tetrahydroisoquinolin-8-amine TFA salt

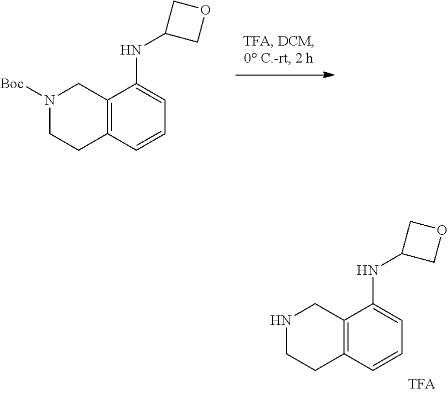 [SMILES: CC(C)(C)OC(=O)N1CCc2cccc(NC3COC3)c2C1 ; c1cc2c(c(NC3COC3)c1)CNCC2]

The title compound was prepared from tert-butyl 8-(oxetan-3-ylamino)-3,4-dihydroisoquinoline-2(1H)-carboxylate (step 1) and TFA analogously to Intermediate N step 3.

LCMS (Method A): 0.759 min, MS: ES+ 205.0 (M+1).

### Intermediate O: (S)—N-(Tetrahydrofuran-3-yl)-1,2,3,4-tetrahydroisoquinolin-8-amine hydrochloride

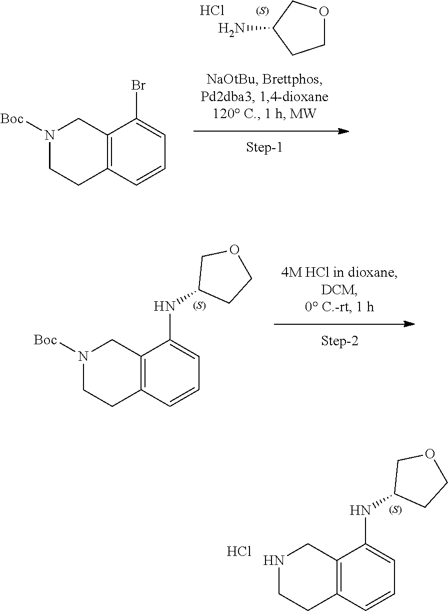

The title compound was prepared from tert-butyl 8-bromo-3,4-dihydroisoquinoline-2(1H)-carboxylate (CAS 893566-75-1) and (S)-tetrahydrofuran-3-amine analogously to Intermediate A steps 1 and 2.

¹H NMR (DMSO-d6, 400 MHz): δ 1.84-1.91 (m, 1H), 2.92-2.95 (m, 2H), 3.25-3.26 (m, 2H), 3.61-3.64 (m, 2H), 3.68-3.74 (m, 2H), 3.82-3.92 (m, 2H), 3.99-4.03 (m, 4H), 6.58-6.64 (m, 2H), 7.03-7.12 (m, 1H), 9.83 (bs, 2H).

LCMS (Method A): 0.882 min, MS: ES+ 219.14 (M+1).

### Intermediate OA: N-(Pyrimidin-5-yl)isoindolin-4-amine hydrochloride

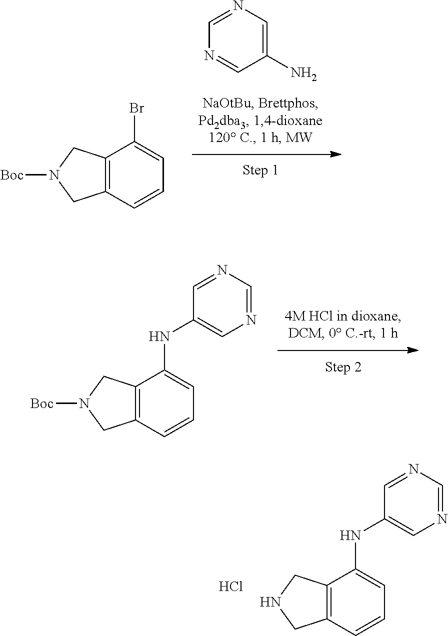 [SMILES: Nc1cncnc1 ; CC(C)(C)OC(=O)N1Cc2cccc(Br)c2C1 ; CC(C)(C)OC(=O)N1Cc2cccc(Nc3cncnc3)c2C1 ; Cl ; c1cc2c(c(Nc3cncnc3)c1)CNC2]

The title compound was prepared from tert-butyl 4-bromoisoindoline-2-carboxylate (Intermediate N step 1) and pyrimidin-5-amine (CAS 591-55-9) analogously to Intermediate A steps 1 and 2.

¹H NMR (DMSO-d6, 400 MHz): δ 4.45-4.52 (m, 4H), 7.03 (d, J=7.2 Hz, 1H), 7.23 (d, J=8 Hz, 1H), 7.31 (t, J=7.6 Hz, 15.6 Hz, 1H), 8.55 (s, 2H), 8.60 (bs, 1H), 8.71 (s, 2H), 9.93 (bs, 2H).

LCMS (Method A): 0.698 min, MS: ES+ 213.2.

### Intermediate OB: N-((Tetrahydrofuran-3-yl)methyl)isoindolin-4-amine

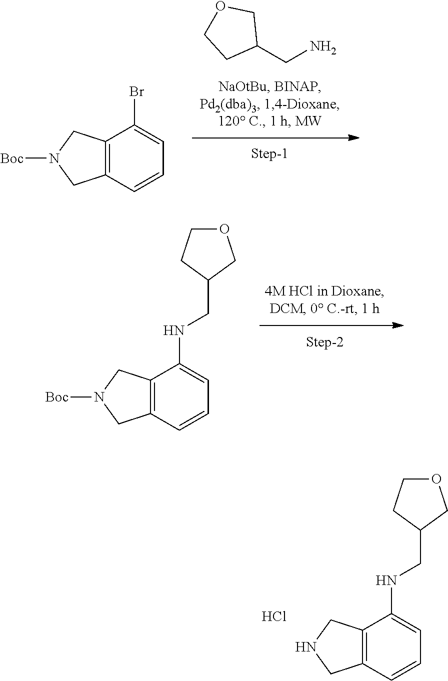 [SMILES: NCC1CCOC1 ; CC(C)(C)OC(=O)N1Cc2cccc(Br)c2C1 ; CC(C)(C)OC(=O)N1Cc2cccc(NCC3CCOC3)c2C1 ; Cl ; c1cc2c(c(NCC3CCOC3)c1)CNC2]

The title compound was prepared from tert-butyl 4-bromoisoindoline-2-carboxylate (Intermediate N step 1) and (tetrahydrofuran-3-yl)methanamine analogously to Intermediate A steps 1 and 2.

¹H NMR (DMSO-d6, 400 MHz): δ 1.24 (s, 2H) 1.58-1.64 (m, 1H), 1.94-2.03 (m, 1H), 3.05-3.07 (m, 2H), 3.47-3.50 (m, 1H), 3.60-3.69 (m, 1H), 3.71-3.78 (m, 2H), 4.30-4.42 (dd, J=10.8 Hz, 5.6 Hz, 4H), 6.56-6.61 (m, 2H), 7.15 (t, J=8 Hz, 7.6 Hz, 1H), 9.84 (s, 1H).

LCMS (Method A): 0.879 min, MS: ES+ 219.0 (M+1).

### Intermediate OC: (R)—N-(Tetrahydrofuran-3-yl)-1,2,3,4-tetrahydroisoquinolin-8-amine hydrochloride

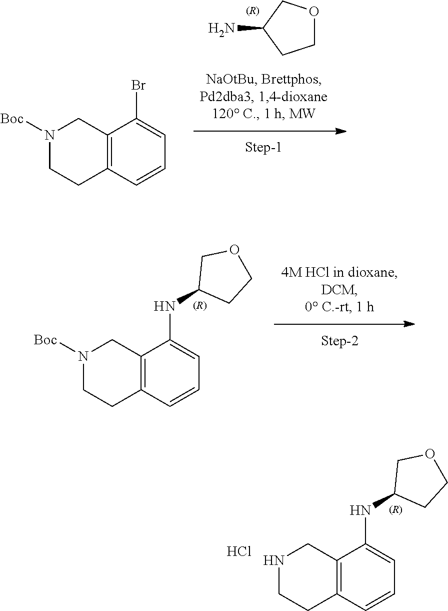

The title compound was prepared from tert-butyl 8-bromo-3,4-dihydroisoquinoline-2(1H)-carboxylate (CAS 893566-75-1) and (R)-tetrahydrofuran-3-amine hydrochloride (CAS 204512-95-8) analogously to Intermediate A steps 1 and 2.

¹H NMR (DMSO-d6, 400 MHz): δ 1.81-1.90 (m, 1H), 2.13-2.22 (m, 1H), 2.92-2.93 (m, 4H), 3.26-3.27 (m, 4H), 3.58-3.60 (m, 2H), 3.70-3.74 (m, 2H), 3.80-3.89 (m, 2H), 3.90-4.01 (m, 2H), 6.52 (d, J=8 Hz, 2H), 6.63-6.75 (m, 1H), 7.03-7.10 (m, 2H), 9.47-9.67 (bs, 2H).

LCMS (Method B): 2.11 min, MS: ES+ 219.3 (M+1)

### Intermediate Q: (R)-1-(3-(Isoindolin-4-ylamino)pyrrolidin-1-yl)ethan-1-one hydrochloride

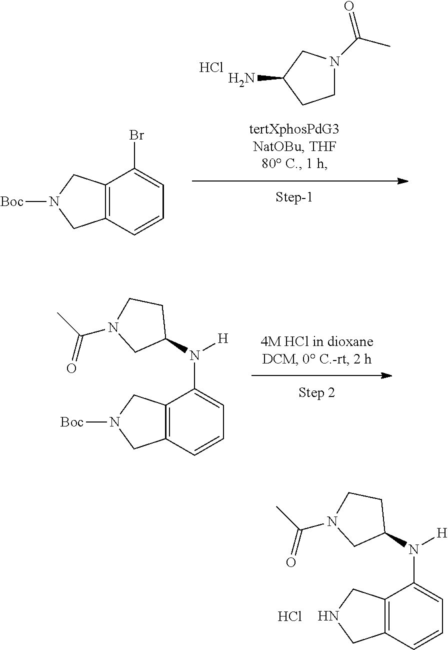

The title compound was prepared from tert-butyl 4-bromoisoindoline-2-carboxylate (Intermediate N step 1), (R)-1-(3-aminopyrrolidin-1-yl)ethan-1-one hydrochloride (CAS 1286208-55-6) and t-BuxPhosPdG3 analogously to Intermediate A steps 1 and 2.

¹H NMR (DMSO-d6, 400 MHz): δ 1.94 (d, J=4.0 Hz, 3H), 2.11-2.23 (m, 2H), 3.24-3.57 (m, 6H), 4.31-4.40 (m, 2H), 4.40-4.43 (m, 2H), 6.58-6.65 (m, 2H), 7.18 (t, J=7.6 Hz, 1H), 9.70 (s, 2H).

LCMS (Method A): 0.778 min, MS: ES+ 245.93 (M+1).

### Intermediate QA: (S)-1-(3-(Isoindolin-4-ylamino)pyrrolidin-1-yl)ethan-1-one

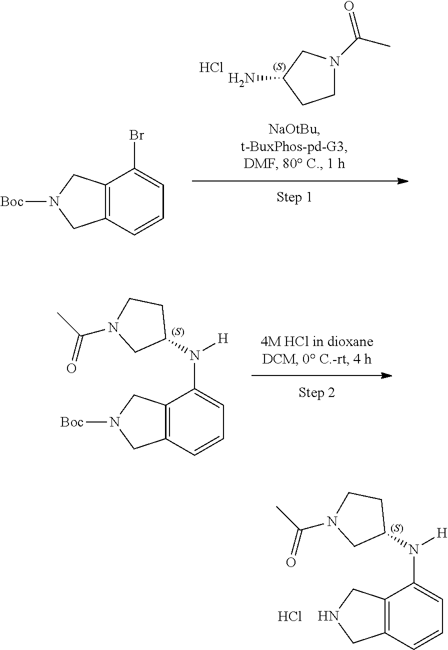

The title compound was prepared from tert-butyl 4-bromoisoindoline-2-carboxylate (Intermediate N step 1) and (S)-1-(3-aminopyrrolidin-1-yl) ethan-1-one hydrochloride (CAS 1286208-19-2) analogously to Intermediate A steps 1 and 2.

¹H NM R (DMSO-d6, 400 MHz): δ 1.86-1.98 (m, 3H), 2.10-2.21 (m, 1H), 2.73 (s, 1H), 2.89 (s, 1H), 3.16 (s, 1H), 3.56 (s, 1H), 4.02-4.13 (m, 1H), 4.33 (d, J=4 Hz, 2H), 4.41 (t, J=4 Hz, 2H), 6.60-6.67 (m, 2H), 7.15-7.20 (m, 1H), 10.06 (s, 2H).

LCMS (Method A): 0.79 min, MS: ES+ 245.8 (M+1)

### Intermediate QB: 1-(4-(Isoindolin-4-ylamino)piperidin-1-yl)ethan-1-one hydrochloride

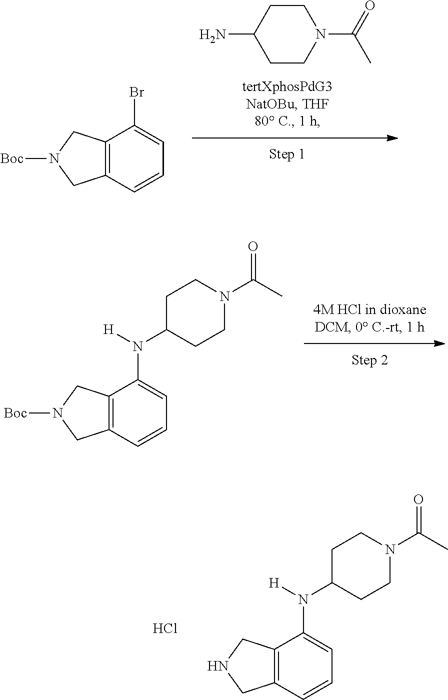

The title compound was prepared from tert-butyl 4-bromoisoindoline-2-carboxylate (Intermediate N step 1) and 1-(4-aminopiperidin-1-yl)ethan-1-one (CAS 160357-94-8) analogously to Intermediate A steps 1 and 2.

¹H NM R (DMSO-d6, 400 MHz): δ 1.37-1.49 (m, 1H), 1.59 (s, 1H), 2.64-2.69 (m, 1H), 3.10-3.15 (m, 1H), 3.57 (s, 3H), 3.82-3.85 (d, J=12 Hz, 1H), 4.32-4.44 (m, 4H), 6.81-6.87 (m, 2H), 7.22-7.26 (t, J=16 Hz, 1H), 10.03-10.04 (bs, 2H).

LCMS (Method A): 0.847 min, MS: ES+ 260.2 (M+1).

### Intermediate QC: (R)-1-(3-(Isoindolin-4-ylamino)piperidin-1-yl)ethan-1-one hydrochloride

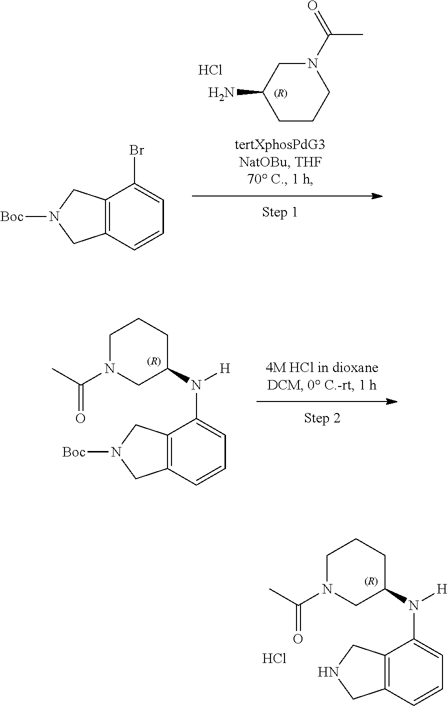

The title compound was prepared from tert-butyl 4-bromoisoindoline-2-carboxylate (Intermediate N step 1) and (R)-1-(3-aminopiperidin-1-yl)ethan-1-one hydrochloride analogously to Intermediate A steps 1 and 2.

¹H NMR (DMSO-d6, 400 MHz): δ 1.57-1.60 (m, 2H), 1.91-1.95 (m, 2H), 2.01-2.02 (m, 3H), 2.89-3.01 (m, 2H), 3.11-3.25 (m, 2H), 3.57 (s, 2H), 4.40 (m, 2H) 4.40-4.54 (m, 1H), 6.60-6.92 (m, 2H), 7.14-7.19 (m, 1H), 8.02-8.11 (m, 2H), 9.57 (s, 2H).

LCMS (Method A): 0.877 min, MS: ES+ 260.1 (M+1).

### Intermediate QD: (S)-1-(3-(Isoindolin-4-ylamino)piperidin-1-yl)ethan-1-one hydrochloride

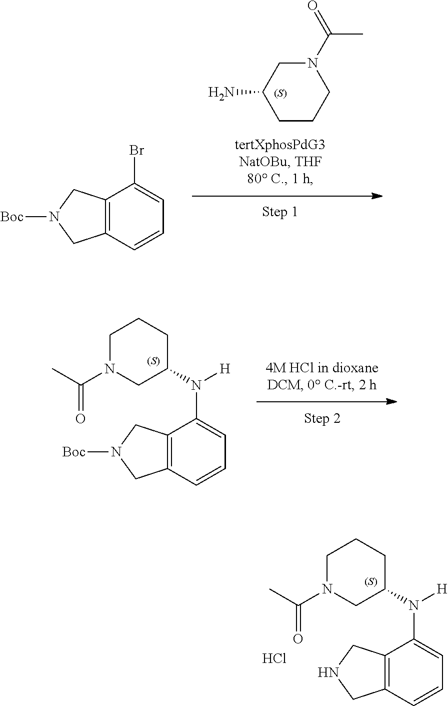

The title compound was prepared from tert-butyl 4-bromoisoindoline-2-carboxylate (Intermediate N step 1) and (S)-1-(3-aminopiperidin-1-yl)ethan-1-one hydrochloride analogously to Intermediate A steps 1 and 2.

¹H NMR (DMSO-d6, 400 MHz): δ 1.57-1.60 (m, 2H), 1.91-1.95 (m, 2H), 2.01-2.02 (m, 3H), 2.89-3.01 (m, 2H), 3.11-3.25 (m, 2H), 3.57 (s, 2H), 4.40 (m, 2H) 4.40-4.54 (m, 1H), 6.60-6.92 (m, 2H), 7.14-7.19 (m, 1H), 8.02-8.11 (m, 2H), 9.57 (s, 2H).

LCMS (Method A): 0.873 min, MS: ES+ 260.08 (M+1).

### Intermediate R: 1-(Isoindolin-5-yl)-N,N-dimethylmethanamine hydrochloride

### Step 1: 2-(tert-Butyl) 5-methyl isoindoline-2,5-dicarboxylate

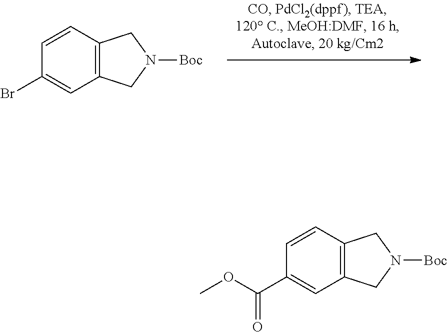 [SMILES: CC(C)(C)OC(=O)N1Cc2ccc(Br)cc2C1 ; COC(=O)c1ccc2c(c1)CN(C(=O)OC(C)(C)C)C2]

A solution of tert-butyl 5-bromoisoindoline-2-carboxylate (CAS 201940-08-1) (50 g, 167.78 mmol, 1 eq.) in MeOH:DMF (450 mL: 50 mL) in a reaction autoclave was treated with TEA (50.83 mL, 502.3 mmol, 3.0 eq.) at room temperature and degassed using N₂ for 15 mins. PdCl₂(dppf) (24.53 g, 33.55 mmol, 0.2 eq.) was added and the reaction mixture was placed under a pressure of 22 kg/cm² of carbon monoxide at room temperature. The mixture was heated to 120° C. for 16 h. The resulting mixture was cooled to room temperature and concentrated under vacuum. The crude material was diluted with EtOAc (500 mL) and washed with ice cold brine solution (3×500 mL). The combined organic extracts were dried over Na₂SO₄, filtered and concentrated under vacuum. The crude material was purified by chromatography on silica (eluting with 20% EtOAc in hexane) to yield the title compound (38 g, Yield: 82%).

¹H NMR (DMSO-d6, 400 MHz): δ 1.46 (s, 9H), 3.86 (s, 3H), 4.63-4.66 (m, 4H), 7.46-7.49 (m, 1H), 7.88-7.94 (q, J=9.2 Hz, 14 Hz, 2H).

LCMS (Method A): 2.153 min, MS: ES+ 221.8 (M−56).

### Step 2: tert-Butyl 5-(hydroxyl methyl) isoindoline-2-carboxylate

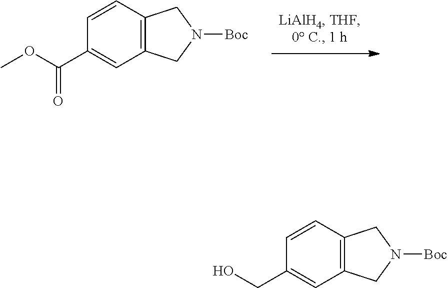 [SMILES: COC(=O)c1ccc2c(c1)CN(C(=O)OC(C)(C)C)C2 ; CC(C)(C)OC(=O)N1Cc2ccc(CO)cc2C1]

To a stirred solution of 2-(tert-butyl) 5-methyl isoindoline-2,5-dicarboxylate (step 1) (38 g, 137.18 mmol, 1 eq.) in THF (380 mL) at 0° C. was added dropwise LiAlH₄ solution (1M in THF) (140.8 mL, 1.0 eq.) and the mixture was stirred at 0° C. for 1 h. The resulting mixture was poured into cold saturated solution of NH₄Cl (400 mL) and extracted with EtOAc (3×500 mL). The combined organic extracts were dried over anhydrous Na₂SO₄, filtered and concentrated under vacuum to yield the title compound (18 g, Yield: 53%). The crude material was used in the next step without further purification.

¹H NMR (DMSO-d6, 400 MHz): δ 1.46 (s, 9H), 4.49 (d, J=5.6 Hz, 2H), 4.55-4.57 (m, 4H), 5.21 (t, J=5.6 Hz, 1H), 7.21-7.28 (m, 3H).

LCMS (Method A): 1.666 min, MS: ES+ 193.9 (M−56).

### Step 3: tert-Butyl 5-(bromomethyl)isoindoline-2-carboxylate

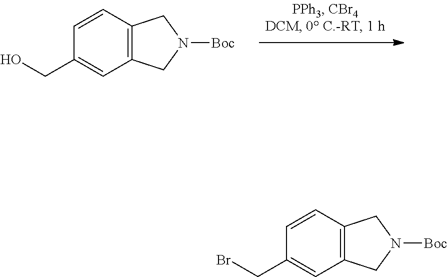 [SMILES: CC(C)(C)OC(=O)N1Cc2ccc(CO)cc2C1 ; CC(C)(C)OC(=O)N1Cc2ccc(CBr)cc2C1]

To a stirred solution of tert-butyl 5-(hydroxyl methyl) isoindoline-2-carboxylate (step 2) (18 g, 72.29 mmol, 1.0 eq.) in DCM (180 mL) were added portionwise triphenyl phosphine (22.72 g, 86.72 mmol, 1.2 eq.) and CBr₄ (28.76 g, 86.73 mmol, 1.2 eq.) at 0° C. The reaction mixture was stirred at room temperature for 1 h. The resulting mixture was concentrated under vacuum and the crude material was purified by chromatography on silica (product was eluted in 20% EtOAc in hexane) to yield the title compound as white solid. (20 g, Yield: 88%)

¹H NMR (DMSO-d6, 400 MHz): δ 1.46 (s, 9H), 4.57 (d, J=8.0 Hz, 4H), 4.73 (s, 2H), 7.30-7.33 (m, 1H), 7.36-7.41 (m, 2H).

LCMS (Method A): 2.341 min, MS: ES+ 255.8 (M−56).

### Step 4: tert-Butyl 5-((dimethylamino)methyl)isoindoline-2-carboxylate

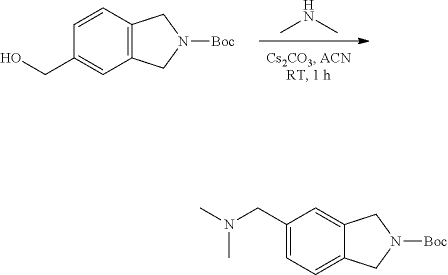

Performed in 4 parallel batches, each of 5 g scale: To a stirred solution of tert-butyl 5-(bromomethyl)isoindoline-2-carboxylate (5 g, 16.02 mmol, 1.0 eq.) in MeCN (50 mL) were added Cs₂CO₃ (15.67 g, 48.06 mmol, 3.0 eq.) and dimethylamine (1M in THF) (CAS 124-40-3) (12.60 g, 104.13 mmol, 6.5 eq.) and the mixture was stirred at room temperature for 1 h. The resulting mixture was concentrated under vacuum and purification of the crude material by flash chromatography yielded the title compound (14 g, Yield: 79%).

¹H NMR (DMSO-d6, 400 MHz): δ 1.46 (s, 9H), 2.18 (s, 6H), 3.45 (s, 2H), 4.56 (d, J=8.0 Hz, 2H), 7.2-7.29 (m, 3H).

LCMS (Method A): 1.195 min, MS: ES+ 277.0 (M+1).

### Step 5: 1-(Isoindolin-5-yl)-N,N-dimethylmethanamine hydrochloride

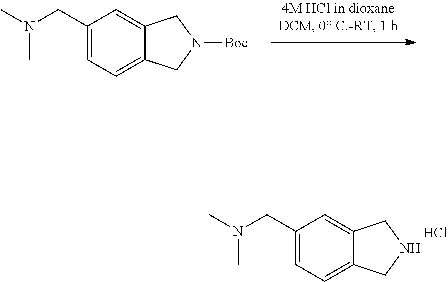 [SMILES: CN(C)Cc1ccc2c(c1)CN(C(=O)OC(C)(C)C)C2 ; CN(C)Cc1ccc2c(c1)CNC2 ; Cl]

To a stirred solution of tert-butyl 5-((dimethylamino)methyl) isoindoline-2-carboxylate (step 4) (14 g, 50.65 mmol, 1.0 eq.) in DCM (140 mL) was added dropwise 4N HCl in dioxane (70 mL) at 0° C. The reaction mixture was allowed to warm to room temperature and stirred for 1 h. The resulting mixture was concentrated under vacuum and the crude material was triturated with diethyl ether (3×40 mL), then n-pentane (3×40 mL) and dried under high vacuum to yield the title compound (12 g, Yield: Quantitative).

¹H NMR (DMSO-d6, 400 MHz): δ 2.67 (d, J=4.8 Hz, 6H), 4.29 (d, J=5.6 Hz, 2H), 4.53 (t, J=5.6 Hz, 4H), 7.59 (d, J=8.0 Hz, 1H), 7.57-7.59 (m, 2H), 10.11 (s, 2H), 10.92 (s, 1H). HCl salt.

LCMS (Method B): 1.37 min, MS: ES+ 177.2 (M+1).

### Intermediate RA: 5-((4-Methyl piperazin-1-yl)methyl)isoindoline HCl salt

### Step 1: tert-Butyl 5-formylisoindoline-2-carboxylate

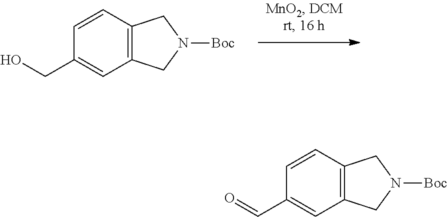 [SMILES: CC(C)(C)OC(=O)N1Cc2ccc(CO)cc2C1 ; CC(C)(C)OC(=O)N1Cc2ccc(C=O)cc2C1]

To a stirred solution of tert-butyl 5-(hydroxymethyl) isoindoline-2-carboxylate (Intermediate R step 2)) (9.0 g, 36.12 mmol, 1.0 eq) in DCM (270 mL) was added activated MnO₂ (90 g) and the reaction mixture was stirred at room temperature for 16 h. The resulting mixture was filtered through Celite® and washed through with 10% MeOH in DCM (3×100 mL). The combined filtrate was concentrated under vacuum and purification of the crude material by chromatography on silica (product was eluted at 8% EtOAc in n-hexane) yielded the title compound. (6.0 g, Yield: 67.21%).

1H NMR (DMSO-d6, 400 MHz): 1.46 (s, 9H), 4.65-4.67 (m, 4H), 4) 7.54-7.57 (m, 1H), 7.84-7.87 (m, 2H), 10.00 (s, 1H).

LCMS (Method A): 1.989 min, MS: ES+ 148.2 (M−100)

### Step 2: tert-Butyl 5-((4-methylpiperazin-1-yl) methyl) isoindoline-2-carboxylate

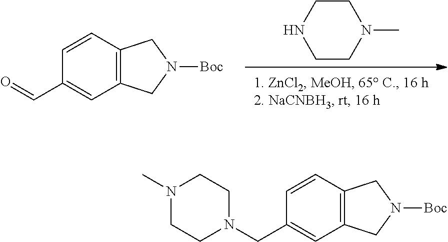

To a stirred solution of tert-butyl 5-formylisoindoline-2-carboxylate (step 1) (4.0 g, 16.17 mmol, 1.0 eq) in MeOH (100 mL) were added N-methyl piperazine (1.61 g, 16.17 mmol, 1.0 eq) and ZnCl₂ (1.04 g, 8.05 mmol, 0.5 eq) at room temperature. The reaction mixture was heated to 70° C. and stirred for 16 h. The mixture was cooled to 0° C., NaCNBH₃ (2.02 g, 32.2 mmol, 2.0 eq) was added and stirring continued at room temperature for 16 h. The resulting mixture was concentrated under vacuum and the crude material was taken up in water (100 mL). The mixture was extracted with EtOAc (3×150 mL) and the combined organic extracts were dried over Na₂SO₄, filtered and concentrated under vacuum. The crude material was purified by chromatography on silica (product was eluted in 6% MeOH in DCM) to yield the title compound (4.3 g, Yield: 80.21%).

1H NMR (DMSO-d6, 400 MHz): 1.45 (s, 9H), 2.23 (s, 3H), 2.40-2.50 (m, 8H) (piperazine proton merged), 2.45 (s, 2H), 4.54-4.56 (m, 4H), 7.19-7.26 (m, 3H).

LCMS (Method A): 0.945 min, MS: ES+ 332.32 (M+1).

### Step 3: 5-((4-Methyl piperazin-1-yl) methyl) isoindoline HCl salt

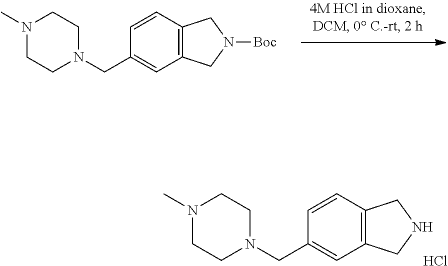 [SMILES: CN1CCN(Cc2ccc3c(c2)CN(C(=O)OC(C)(C)C)C3)CC1 ; CN1CCN(Cc2ccc3c(c2)CNC3)CC1 ; Cl]

The title compound was prepared from tert-butyl 5-((4-methyl piperazin-1-yl)methyl) isoindoline-2-carboxylate (step 2) and 4M HCl in dioxane analogously to Intermediate R step 5.

¹H NMR (DMSO-d6, 400 MHz): 2.78 (s, 3H), 3.47-3.59 (m, 2H), 3.82-3.84 (m, 4H), 4.32-4.35 (m, 2H), 4.51 (s, 4H), 7.48 (d, J=7.6 Hz, 1H), 7.64 (s, 2H), 10.15 (s, 1H)

LCMS (Method B): 1.33 min, MS: ES+ 232.37 (M+1)

### Intermediate S: 4-(Isoindolin-4-ylamino)dihydrofuran-2(3H)-one hydrochloride

### Step 1: tert-Butyl 4-((5-oxotetrahydrofuran-3-yl)amino)isoindoline-2-carboxylate

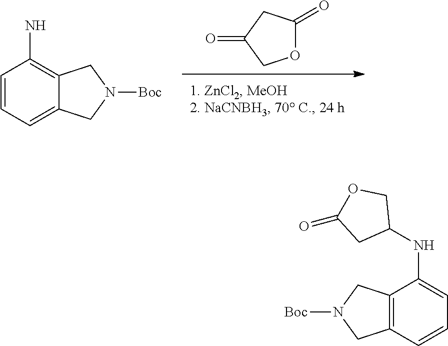 [SMILES: CC(C)(C)OC(=O)N1Cc2cccc(N)c2C1 ; O=C1COC(=O)C1 ; CC(C)(C)OC(=O)N1Cc2cccc(NC3COC(=O)C3)c2C1]

Performed in 2 parallel batches, each of 0.5 g scale: To a stirred solution of tert-butyl 4-aminoisoindoline-2-carboxylate (0.5 g, 2.13 mmol, 1.0 eq.) in MeOH (10 mL) were added furan-2,4(3H,5H)-dione (CAS 4971-56-6) (0.32 g, 3.20 mmol, 1.5 eq.) and Zinc chloride (0.87 g, 6.41 mmol, 3 eq.) and the mixture was stirred at room temperature for 16 h. NaCNBH₃ (0.67 g, 10.68 mmol, 5 eq.) was added to the reaction mixture at room temperature. The resulting mixture was heated to 70° C. and stirred for 16 h. The reaction mixture was cooled to room temperature and diluted with water (20 mL) and extracted with EtOAc (3×30 mL). The combined organic extracts were dried over Na₂SO₄, filtered and concentrated under vacuum. The crude material (1.2 g) was purified using flash chromatography (product eluted at 2% MeOH:DCM) to yield the title compound as an off-white solid (0.4 g, Yield: 29.4%).

¹H NMR (DMSO-d6, 400 MHz): δ 1.46 (d, J=8 Hz, 9H), 2.94-3.07 (m, 1H), 3.17 (d, J=4 Hz, 1H), 4.10-4.18 (m, 1H), 4.32-4.40 (m, 2H), 4.47 (s, 2H), 5.12 (d, J=8 Hz, 1H), 6.43-6.48 (m, 1H), 6.63 (t, J=8 Hz, 1H), 7.08-7.12 (m, 1H).

LCMS (Method A): 1.962 min, MS: ES-218.9 (M−100).

### Step 2: 4-(Isoindolin-4-ylamino)dihydrofuran-2(3H)-one hydrochloride

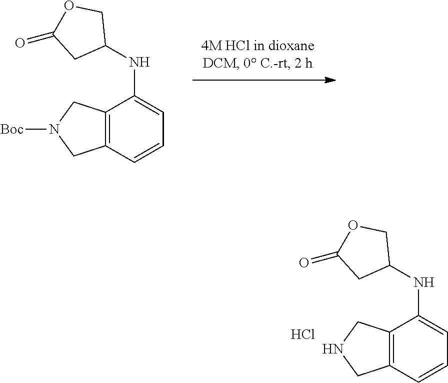 [SMILES: CC(C)(C)OC(=O)N1Cc2cccc(NC3COC(=O)C3)c2C1 ; Cl ; O=C1CC(Nc2cccc3c2CNC3)CO1]

To a stirred solution of tert-butyl 4-((5-oxotetrahydrofuran-3-yl)amino)isoindoline-2-carboxylate (0.4 g, 1.25 mmol, 1.0 eq.) in DCM (4 mL) was cooled to 0° C. 4M HCl in dioxane (2 mL) was added dropwise to reaction mixture at 0° C. The reaction mixture was stirred at room temperature for 2 h. The reaction mixture was concentrated under vacuum. The crude material was triturated with diethyl ether (3×5 mL). The solid material was dried under high vacuum to yield the title compound as a white solid (0.4 g, Quantitative).

¹H NM R (DMSO-d6, 400 MHz): δ 2.99-3.05 (m, 1H), 3.57 (s, 2H), 4.13-4.19 (m, 1H), 4.37-4.30 (m, 3H), 4.32-4.41 (m, 3H), 4.48 (d, J=4 Hz, 1H), 6.53 (d, J=8 Hz, 1H), 6.68 (d, J=8 Hz, 1H), 7.17 (d, J=8 Hz, 1H), 9.93 (s, 1H).

LCMS (Method A): 0.733 min, MS: ES+ 218.9 (M+1).

### Intermediate T: 1-(6,7-Dihydro-5H-pyrrolo[3,4-b]pyridin-2-yl)-N, N-dimethylmethanamine hydrochloride

### Step 1: tert-Butyl 2-chloro-5,7-dihydro-6H-pyrrolo[3,4-b]pyridine-6-carboxylate

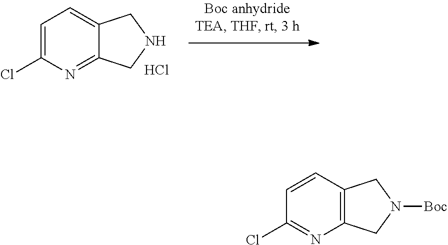 [SMILES: Clc1ccc2c(n1)CNC2 ; Cl ; CC(C)(C)OC(=O)N1Cc2ccc(Cl)nc2C1]

The title compound was prepared from 2-chloro-6,7-dihydro-5H-pyrrolo[3,4-b]pyridine hydrochloride (CAS 1841081-37-5) and Boc-anhydride analogously to Intermediate H step 2.

¹H NMR (DMSO-d6, 400 MHz): δ 1.43-1.46 (m, 9H), 4.53-4.61 (m, 4H), 7.43 (d, J=8.4 Hz, 1H), 7.83 (t, J=7.2 Hz, 1H).

LCMS (Method A): 1.937 min, MS: ES+ 254.78 (M+1).

### Step 2: 6-(tert-Butyl) 2-ethyl 5,7-dihydro-6H-pyrrolo[3,4-b]pyridine-2,6-dicarboxylate

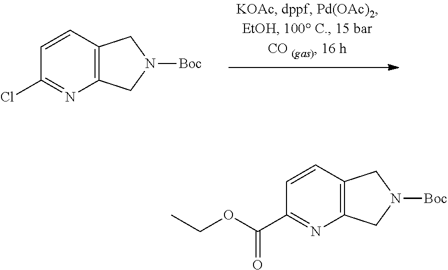 [SMILES: CC(C)(C)OC(=O)N1Cc2ccc(Cl)nc2C1 ; CCOC(=O)c1ccc2c(n1)CN(C(=O)OC(C)(C)C)C2]

To a stirred solution of tert-butyl 2-chloro-5,7-dihydro-6H-pyrrolo[3,4-b]pyridine-6-carboxylate (step 1) (4.5 g, 17.7 mmol, 1.0 eq) in EtOH (90 mL) were added DPPF (0.29 g, 0.53 mmol, 0.03 eq), KOAc (3.47 g, 35 mmol, 2.0 eq) and Pd(OAc)₂ (0.19 g, 0.88 mmol, 0.05 eq) at room temperature. The reaction mixture was heated to 100° C. under an atmosphere of CO_{(g)} (15 bar pressure) for 16 h. The resulting mixture was cooled to room temperature, filtered with Celite® and washed through with EtOAc (200 mL). The filtrate was concentrated under vacuum and the crude material was dissolved in EtOAc (200 mL) and washed with brine solution (2×50 mL). The organic layer was dried over Na₂SO₄, filtered and concentrated under vacuum. The crude material was purified by chromatography on silica (20% EtOAc in hexane) to yield the title compound (3.8 g, 73.46%).

¹H NMR (DMSO-d6, 400 MHz): δ 1.33 (t, J=7.2 Hz, 3H), 1.47 (s, 9H), 4.32-4.37 (m, 2H), 4.60-4.70 (m, 4H), 7.94-8.00 (m, 2H),

LCMS (Method A): 1.749 min, MS: ES+ 292.89 (M+1).

### Step 3: tert-Butyl 2-(hydroxymethyl)-5,7-dihydro-6H-pyrrolo[3,4-b]pyridine-6-carboxylate

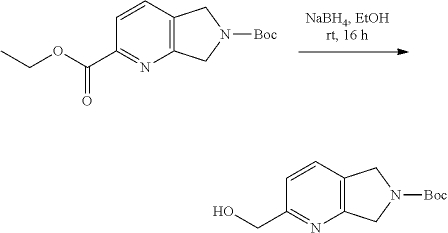 [SMILES: CCOC(=O)c1ccc2c(n1)CN(C(=O)OC(C)(C)C)C2 ; CC(C)(C)OC(=O)N1Cc2ccc(CO)nc2C1]

To a stirred solution of 6-(tert-butyl) 2-ethyl 5,7-dihydro-6H-pyrrolo[3,4-b]pyridine-2,6-dicarboxylate (step 2) (3.8 g, 13 mmol, 1.0 eq) in EtOH (76 mL) was added portion wise NaBH₄ (2.95 g, 78 mmol, 6.0 eq) and the reaction mixture was stirred at room temperature for 16 h. The resulting mixture was concentrated under vacuum, the crude material was diluted with EtOAc (200 mL) and washed with a brine solution (2×50 mL). The organic portion was dried over Na₂SO₄, filtered and concentrated under vacuum. The crude material was purified by chromatography on silica (3% MeOH in DCM) to yield the title compound (3.0 g, 92.2%).

¹H NMR (DMSO-d6, 400 MHz): δ 1.46 (s, 9H), 4.50-4.61 (m, 6H), 5.43-5.46 (m, 1H), 7.37 (d, J=8 Hz, 1H), 7.74 (t, J=7.6 Hz, 1H), 4.46-4.48 (m, 2H), 4.56-4.59 (m, 4H), 5.16-5.20 (m, 2H), 7.21-7.28 (m, 3H).

LCMS (Method A): 1.264 min, MS: ES+ 250.83 (M+1).

### Step 4: tert-Butyl 2-(bromomethyl)-5,7-dihydro-6H-pyrrolo[3,4-b]pyridine-carboxylate

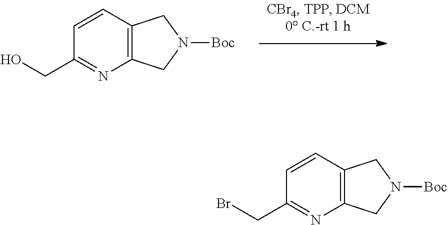 [SMILES: CC(C)(C)OC(=O)N1Cc2ccc(CO)nc2C1 ; CC(C)(C)OC(=O)N1Cc2ccc(CBr)nc2C1]

To a cooled (0° C.) stirred solution of tert-butyl 2-(hydroxymethyl)-5,7-dihydro-6H-pyrrolo[3,4-b]pyridine-6-carboxylate (step 3) (1.0 g, 4 mmol, 1.0 eq) in DCM (15 mL) was added triphenyl phosphate (1.26 g, 4.8 mmol, 1.2 eq) followed by portion wise addition of CBr₄ (1.59 g, 4.8 mmol, 1.2 eq). The reaction mixture was allowed to warm to room temperature and stirred for 1 h. The resulting mixture was concentrated under vacuum and purification of the crude material by chromatography on silica (product eluted at 20% EtOAc in hexane) yielded the title compound (0.85 g, 68%).

¹H NMR (DMSO-d6, 400 MHz): δ 1.46-1.47 (m, 9H), 4.53-4.63 (m, 4H), 4.70 (s, 2H), 7.46 (d, J=8 Hz, 1H), 7.77 (s, 1H), 4.61 (d, J=10.4 Hz, 2H), 4.83 (d, J=8 Hz, 2H), 7.56-7.90 (m, 3H), 10.06 (d, J=5.6 Hz, 1H).

LCMS (Method A): 1.838 min, MS: ES+ 312.7 (M+1).

### Step 5: tert-Butyl 2-((dimethylamino)methyl)-5,7-dihydro-6H-pyrrolo[3,4-b]pyridine-6-carboxylate

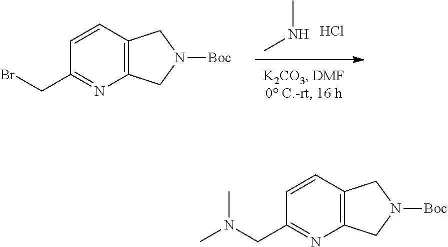

To a stirred solution of dimethyl amine hydrochloride (0.65 g, 7.9 mmol, 2.5 eq) in DMF (10 mL) was added K₂CO₃ (3.09 g, 22.4 mmol, 7.0 eq) at 0° C. and the mixture was stirred for 5 mins. tert-Butyl 2-(bromomethyl)-5,7-dihydro-6H-pyrrolo[3,4-b]pyridine-6-carboxylate (step 4) (1.0 g, 3.2 mmol, 1.0 eq) was added and the reaction mixture was allowed to warm to room temperature, stirring for 16 h. The resulting mixture was poured into water (100 mL) and extracted with EtOAc (2×100 mL). The combined organic extracts were washed with brine solution (2×50 mL), dried over Na₂SO₄, filtered and concentrated under vacuum. The crude material was purified by chromatography on silica (7% MeOH in DCM) to yield the title compound (0.3 g, 33%).

¹H NMR (DMSO-d6, 400 MHz): δ 1.46 (s, 9H), 2.18 (s, 6H), 3.50 (s, 2H), 4.51-4.61 (m, 4H), 7.33 (d, J=8 Hz, 1H), 7.72 (t, J=5.6 Hz, 1H).

LCMS (Method A): 1.042 min, MS: ES+ 278.04 (M+1).

### Step 6: 1-(6,7-Dihydro-5H-pyrrolo[3,4-b]pyridin-2-yl)-N, N-dimethylmethanamine hydrochloride

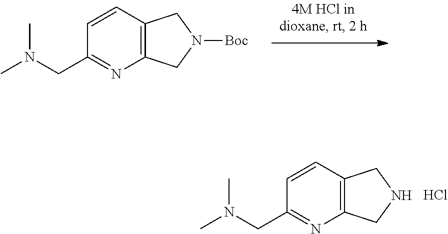 [SMILES: CN(C)Cc1ccc2c(n1)CN(C(=O)OC(C)(C)C)C2 ; CN(C)Cc1ccc2c(n1)CNC2 ; Cl]

The title compound was prepared from tert-butyl 2-((dimethylamino)methyl)-5,7-dihydro-6H-pyrrolo[3,4-b]pyridine-6-carboxylate (step 5) and 4M HCl in dioxane analogously to Intermediate S step 2.

¹H NMR (DMSO-d6, 400 MHz): δ 2.77 (d, J=4.8 Hz, 6H), 4.45 (d, J=5.2 Hz, 2H), 4.51 (t, J=5.6 Hz, 2H), 4.60 (t, J=5.6 Hz, 2H), 7.65 (d, J=8.0 Hz, 1H), 7.97 (d, J=8.0 Hz, 1H).

LCMS (Method B): 1.38 min, MS: ES+ 178.2 (M+1).

### Intermediate U: 2-((8-(Difluoromethyl)-1,2,3,4-tetrahydroisoquinolin-6-yl)oxy)-N,N-dimethylethan-1-amine hydrochloride

### Step 1: tert-Butyl 6-(2-(dimethylamino)ethoxy)-8-vinyl-3,4-dihydroisoquinoline-2(1H)-carboxylate

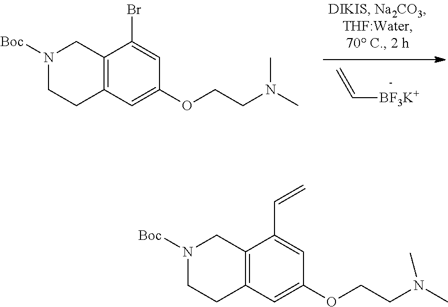

Performed in 3 parallel batches, each of 0.5 g scale: To a solution of tert-butyl 8-bromo-6-(2-(dimethylamino)ethoxy)-3,4-dihydroisoquinoline-2(1H)-carboxylate (Intermediate M step 1) (0.5 g, 1.25 mmol, 1 eq) in THF:H₂O (4:1) (5 mL) were added potassium trifluoro(vinyl)borate (0.251 g, 1.87 mmol, 1.5 eq) and Na₂CO₃ (0.93 g, 8.77 mmol, 7 eq) at room temperature. The resulting mixture was degassed with nitrogen for 10 min. Pd(PPh₃)₂Cl₂ (0.044 g, 0.063 mmol, 0.05 eq) was added and the reaction mixture was heated to 70° C. for 2 h. The resulting mixture was poured into ice cold water (100 mL) and extracted with EtOAc (3×100 mL). The combined organic extracts were dried over Na₂SO₄, filtered and concentrated under vacuum. The crude material was purified by chromatography on silica (eluting with 2% MeOH:DCM) yielding tert-butyl 6-(2-(dimethylamino)ethoxy)-8-vinyl-3,4-dihydroisoquinoline-2(1H)-carboxylate as a yellow solid (1 g, Yield: 77%).

¹H NMR (DMSO-d6, 400 MHz) (D2O Exchange): δ 1.42 (s, 9H), 2.21 (s, 6H), 2.50-2.61 (m, 2H), 2.74 (t, J=5.6 Hz, 11.2 Hz, 2H), 3.51 (t, J=5.6 Hz, 11.6 Hz, 2H), 4.04 (t, J=6.0 Hz, 11.6 Hz, 2H), 4.45 (s, 2H), 5.36 (d, J=14.0 Hz, 1H), 5.75 (d, J=17.2 Hz, 1H), 6.71 (s, 1H), 6.77-6.80 (m, 1H), 6.93 (s, 1H).

LCMS (Method A): 1.372 min, MS ES+: 347.0 (M+1).

### Step 2: tert-Butyl 6-(2-(dimethylamino)ethoxy)-8-formyl-3,4-dihydroisoquinoline-2(1H)-carboxylate

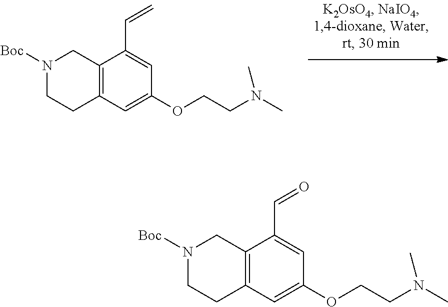 [SMILES: C=Cc1cc(OCCN(C)C)cc2c1CN(C(=O)OC(C)(C)C)CC2 ; CN(C)CCOc1cc(C=O)c2c(c1)CCN(C(=O)OC(C)(C)C)C2]

To a solution of tert-butyl 6-(2-(dimethylamino)ethoxy)-8-vinyl-3,4-dihydroisoquinoline-2(1H)-carboxylate (step 1) (1 g, 2.89 mmol, 1 eq) in 1,4-dioxane (10 mL) was added dropwise a solution of K₂OsO₄ (0.011 g, 0.030 mmol, 0.01 eq) in water (1 mL) and the reaction mixture was stirred at room temperature for 10 min. To this mixture as added dropwise a solution of NalO₄ (1.54 g, 7.19 mmol, 2.5 eq) in water (1 mL) and the mixture was stirred at room temperature for 30 min. The resulting mixture was poured into ice cold water (100 mL) and extracted with EtOAc (3×100 mL) and 10% MeOH:DCM (3×50 mL). The combined organic extracts were dried over Na₂SO₄, filtered and concentrated under vacuum. The crude material was purified by chromatography on silica (eluting with 3% MeOH:DCM) yielding tert-butyl 6-(2-(dimethylamino)ethoxy)-8-formyl-3,4-dihydroisoquinoline-2(1H)-carboxylate as yellow oil (0.7 g, Yield: 70%).

¹H NMR (DMSO-d6, 400 MHz) (D2O Exchange): δ 1.42 (s, 9H), 2.24 (s, 6H), 2.67 (t, J=4.8 Hz, 10.4 Hz, 2H), 2.83 (t, J=6.0 Hz, 11.6 Hz, 2H), 3.55 (t, J=4.8 Hz, 10.4 Hz, 2H), 4.12 (t, J=6.0 Hz, 11.6 Hz, 2H), 4.82 (s, 2H), 7.11 (s, 1H), 7.43 (d, J=2.4 Hz, 1H), 10.05 (s, 1H).

LCMS (Method N): 1.811 min, MS ES+: 348.9 (M+1).

### Step 3: tert-Butyl 8-(difluoromethyl)-6-(2-(dimethylamino)ethoxy)-3,4-dihydroisoquinoline-2(1H)-carboxylate

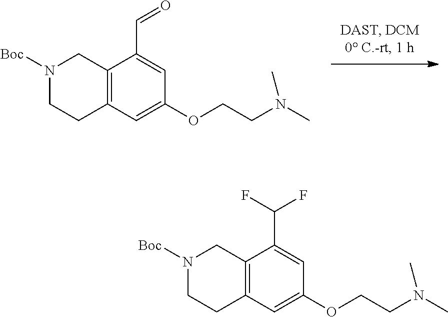 [SMILES: CN(C)CCOc1cc(C=O)c2c(c1)CCN(C(=O)OC(C)(C)C)C2 ; CN(C)CCOc1cc2c(c(C(F)F)c1)CN(C(=O)OC(C)(C)C)CC2]

To a stirred solution of tert-butyl 6-(2-(dimethylamino)ethoxy)-8-formyl-3,4-dihydroisoquinoline-2(1H)-carboxylate (step 2) (0.7 g, 2.01 mmol, 1 eq) in DCM (7 mL) was added dropwise DAST (1.62 g, 10.06 mmol, 5 eq) at 0° C. The reaction mixture was stirred at room temperature for 16 h. The resulting mixture was poured into ice cold water (30 mL) and neutralized into NaHCO₃ solution. The aqueous layer was extracted with DCM (3×30 mL) and the combined organic extracts were dried over Na₂SO₄, filtered and concentrated under vacuum. The crude material was purified by chromatography on silica (eluting with 5% MeOH in DCM) yielding tert-butyl 8-(difluoromethyl)-6-(2-(dimethylamino)ethoxy)-3,4-dihydroisoquinoline-2(1H)-carboxylate as brown oil (0.4 g, Yield: 54%).

¹H NMR (DMSO-d6, 400 MHz): δ 1.42 (s, 9H), 2.26 (s, 6H), 2.69 (t, J=5.6 Hz, 11.2 Hz, 2H), 2.81 (t, J=5.6 Hz, 11.6 Hz, 2H), 3.54 (t, J=5.6 Hz, 11.2 Hz, 2H), 4.08 (t, J=5.6 Hz, 11.2 Hz, 2H), 4.58 (s, 2H), 5.77 (s, 1H), 6.95-7.07 (m, 3H).

### Step 4: 2-((8-(Difluoromethyl)-1,2,3,4-tetrahydroisoquinolin-6-yl)oxy)-N,N-dimethylethan-1-amine hydrochloride

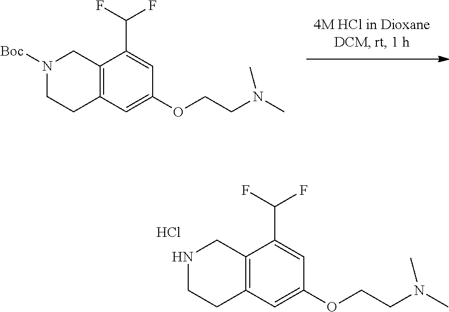 [SMILES: CN(C)CCOc1cc2c(c(C(F)F)c1)CN(C(=O)OC(C)(C)C)CC2 ; Cl ; CN(C)CCOc1cc2c(c(C(F)F)c1)CNCC2]

To a stirred solution of tert-butyl 8-(difluoromethyl)-6-(2-(dimethylamino)ethoxy)-3,4-dihydroisoquinoline-2(1H)-carboxylate (step 3) (0.4 g, 1.08 mmol, 1 eq) in DCM (4 mL) was added dropwise 4M HCl in dioxane (4 mL) at 0° C. The reaction mixture allowed to stir at room temperature for 1 h. The resulting mixture was concentrated under reduced pressure. The crude material was triturated with diethyl ether (10 mL) and n-pentane (10 mL) followed by high vacuum drying yielding 2-((8-(difluoromethyl)-1,2,3,4-tetrahydroisoquinolin-6-yl)oxy)-N,N-dimethylethan-1-amine hydrochloride as brown solid (0.4 g, Yield: Quantitative, 1.30 mmol).

¹H NMR (DMSO-d6, 400 MHz): δ 2.83 (d, J=4.8 Hz, 6H), 3.07-3.08 (m, 2H), 3.50-3.55 (m, 4H), 4.20-4.29 (m, 2H), 4.39-4.42 (m, 2H), 7.04-7.17 (m, 3H). HCl salt

### Intermediate V: 5-((1-Methylpiperidin-4-yl)oxy)isoindoline hydrochloride

### Step 1: tert-Butyl 5-(4,4,5,5-tetramethyl-1,3,2-dioxaborolan-2-yl)isoindoline-2-carboxylate

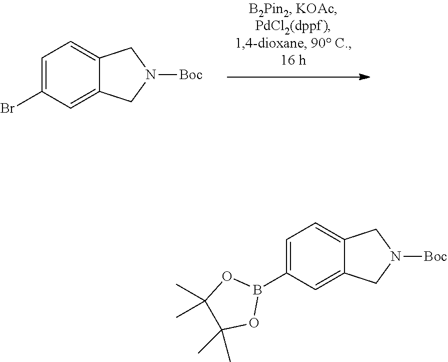 [SMILES: CC(C)(C)OC(=O)N1Cc2ccc(Br)cc2C1 ; CC(C)(C)OC(=O)N1Cc2ccc(B3OC(C)(C)C(C)(C)O3)cc2C1]

To a stirred solution of tert-butyl 5-bromoisoindoline-2-carboxylate (CAS 201940-08-1) (10 g, 33.67 mmol, 1 eq) in 1,4-dioxane (100 mL) were added KOAc (6.59 g, 67.24 mmol, 2.0 eq) and bispinacoleto diborane (9.66 g, 38.03 mmol, 1.13 eq) at room temperature. The reaction mixture was degassed with nitrogen for 30 mins, PdCl₂(dppf) (4.9 g, 6.70 mmol, 0.2 eq) was added and the reaction mixture was heated to 90° C. for 16 h. The resulting mixture poured into ice cold water (200 mL) and extracted with EtOAc (4×100 mL). The combined organic extracts were dried over Na₂SO₄, filtered, and concentrated under vacuum. The crude material was purified by chromatography on silica (eluting with 3.5% EtOAc in hexane) to yield the title compound as a yellow solid (9.0 g, Yield: 77%).

¹H NMR (DMSO-d6, 400 MHz): δ 1.29-1.33 (m, 12H), 1.46 (s, 9H), 4.58-4.61 (m, 4H), 7.34 (d, J=6.8 Hz, 13.6 Hz, 1H), 7.57-7.64 (m, 2H).

LCMS (Method A): 2.578 min, MS ES+: 289.8 (M−56).

### Step 2: tert-Butyl 5-hydroxyisoindoline-2-carboxylate

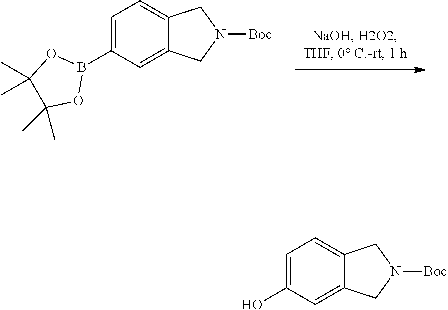 [SMILES: CC(C)(C)OC(=O)N1Cc2ccc(B3OC(C)(C)C(C)(C)O3)cc2C1 ; CC(C)(C)OC(=O)N1Cc2ccc(O)cc2C1]

To a stirred solution of tert-butyl 5-(4,4,5,5-tetramethyl-1,3,2-dioxaborolan-2-yl)isoindoline-2-carboxylate (step 1) (7.5 g, 21.73 mmol, 1 eq) in THF (75 mL), cooled to 0° C., was added NaOH (1.74 g, 43.5 mmol, 2 eq) followed by dropwise addition of H₂O₂ (30% solution, 10.34 g, 304.12 mmol, 4.2 eq). The reaction mixture was allowed to warm to room temperature and stirred for 1 h. The resulting mixture was poured into ice cold water (200 mL) and extracted with EtOAc (3×500 mL). The combined organic extracts were dried over Na₂SO₄, filtered and concentrated under vacuum. The crude material was purified by chromatography on silica (eluting with 25% EtOAc in Hexane) to yield the title compound as an off white solid (5 g, Yield: 98%).

¹H NMR (DMSO-d6, 400 MHz): δ 1.45 (s, 9H), 4.47 (t, J=13.6 Hz, 25.6 Hz, 4H), 6.66-6.69 (m, 2H), 7.08-7.11 (m, 1H), 9.39 (s, 1H).

LCMS (Method A): 1.661 min, MS ES+: 179.8 (M−56).

### Step 3: tert-Butyl 5-((1-methylpiperidin-4-yl)oxy)isoindoline-2-carboxylate

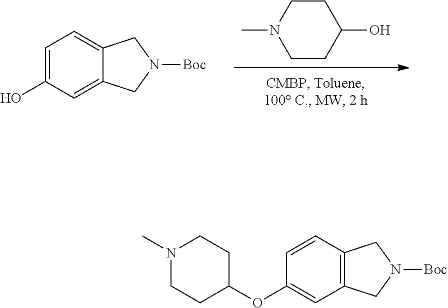

To a stirred solution of tert-butyl 5-hydroxyisoindoline-2-carboxylate (step 2) (0.2 g, 0.85 mmol, 1 eq) and 1-methylpiperidin-4-ol (CAS: 106.82-5) (0.098 g, 0.85 mmol, 1 eq) in toluene (2 mL) was added cyanomethylene)tributylphosphorane (0.41 g, 1.70 mmol, 2 eq) and the reaction mixture was was heated to 100° C. using microwave radiation for 2 h. The resulting mixture was concentrated under vacuum and the crude material was purified twice by chromatography on silica (eluting with 20% MeOH in DCM) to yield the title compound as a brown oil (0.3 g, Yield: quantitative).

¹H NMR (DMSO-d6, 400 MHz): δ 1.44-1.45 (m, 9H), 1.90-1.95 (m, 3H), 2.20-2.30 (m, 5H), 2.60-2.70 (m, 2H), 3.40-3.45 (m, 1H), 4.34 (s, 1H), 4.48-4.54 (m, 4H), 6.85-6.94 (m, 2H), 7.18-7.22 (m, 1H).

LCMS (Method A): 1.344 min, MS ES+: 333.2 (M+1).

### Step 4: 5-((1-Methylpiperidin-4-yl)oxy)isoindoline hydrochloride

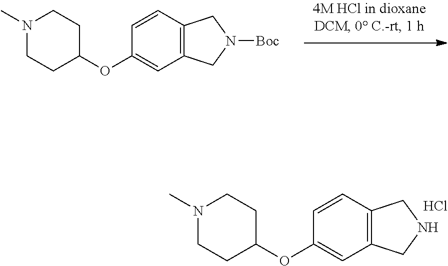 [SMILES: CN1CCC(Oc2ccc3c(c2)CN(C(=O)OC(C)(C)C)C3)CC1 ; CN1CCC(Oc2ccc3c(c2)CNC3)CC1 ; Cl]

To a stirred solution of tert-butyl 5-((1-methylpiperidin-4-yl)oxy)isoindoline-2-carboxylate (step 3) (0.25 g, 0.75 mmol, 1 eq) in DCM (2.5 mL), cooled to 0° C., was added dropwise 4M HCl in dioxane (2.5 mL). The reaction mixture was allowed to warm to room temperature and stirred for 1 h. The resulting mixture was concentrated under vacuum and the crude material was triturated with diethyl ether (10 mL) and n-pentane (10 mL) followed by high vacuum drying to yield the title compound as a yellow oil (0.25 g, Yield: 82%).

¹H NMR (DMSO-d6, 400 MHz): δ 2.10-2.25 (m, 2H), 2.72-2.75 (m, 3H), 3.04-3.18 (m, 2H), 3.20-3.30 (m, 1H), 3.40-3.50 (m, 1H), 3.58 (s, 2H), 4.39-4.44 (m, 3H), 4.50-4.60 (m, 1H), 4.74 (s, 1H), 6.96-7.09 (m, 2H), 7.32 (t, J=8 Hz, 15.6 Hz, 1H), 10.12 (s, 2H).

LCMS (Method A): 0.175 min, MS ES+: 233.0 (M+1).

### Intermediate VA: (R)-5-((1-Methylpyrrolidin-3-yl)oxy)isoindoline hydrochloride

### Step 1: tert-Butyl (R)-5-((1-methylpyrrolidin-3-yl)oxy)isoindoline-2-carboxylate

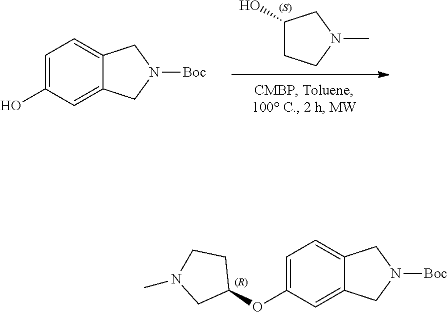

The title compound was prepared from tert-butyl 5-hydroxyisoindoline-2-carboxylate (Intermediate V step 2) and (S)-1-methylpyrrolidin-3-ol (CAS: 104641-59-3) analogously to Intermediate V step 3.

¹H NMR (DMSO-d6, 400 MHz): δ 1.45 (s, 9H), 1.71-1.77 (m, 1H), 2.23-2.39 (m, 5H), 2.57-2.68 (m, 2H), 2.75-2.79 (m, 1H), 4.47-4.57 (m, 4H), 4.82-4.83 (m, 1H), 6.78-6.80 (m, 1H), 6.85 (s, 1H), 7.18-7.22 (m, 1H).

LCMS (Method N): 1.23 min, MS: ES+ 319.1 (M+1)

### Step 2: (R)-5-((1-Methylpyrrolidin-3-yl)oxy)isoindoline hydrochloride

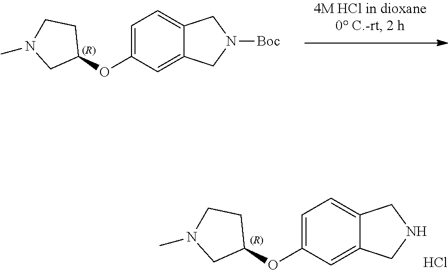

The title compound was prepared from tert-Butyl (R)-5-((1-methylpyrrolidin-3-yl)oxy)isoindoline-2-carboxylate (step 1) and 4M HCl in dioxane analogously to Intermediate V step 4.

1H NMR (DMSO-d6, 400 MHz): δ 2.54-2.59 (m, 1H), 2.83-2.87 (dd, J=4.8 Hz, J=4.8 Hz, 3H), 3.12-3.20 (m, 1H), 3.38-3.40 (m, 1H), 3.57 (s, 1H), 3.61-3.69 (m, 2H), 3.91-4.02 (m, 1H), 4.44-4.46 (m, 4H), 5.11-5.16 (m, 1H), 6.96 (d, J=8.4 Hz, 1H), 7.03-7.04 (m, 1H), 7.33 (d, J=8.4 Hz, 1H), 10.09 (s, 1H).

LCMS (Method N): 1.37 min, MS ES+: 219.3 (M+1).

### Intermediate VB: (S)-5-((1-Methylpyrrolidin-3-yl)oxy)isoindoline hydrochloride

### Step 1: tert-Butyl (S)-5-((1-methylpyrrolidin-3-yl)oxy)isoindoline-2-carboxylate

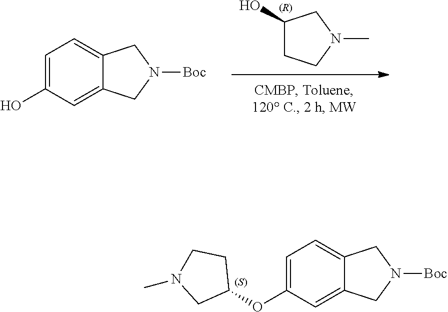

The title compound was prepared from tert-butyl 5-hydroxyisoindoline-2-carboxylate (Intermediate V step 2) and (R)-1-methylpyrrolidin-3-ol (CAS: 104641-60-3) analogously to Intermediate V step 3.

LCMS (Method N): 1.259 min, MS: ES+ 319.1 (M+1).

### Step 2: (S)-5-((1-Methylpyrrolidin-3-yl)oxy)isoindoline hydrochloride

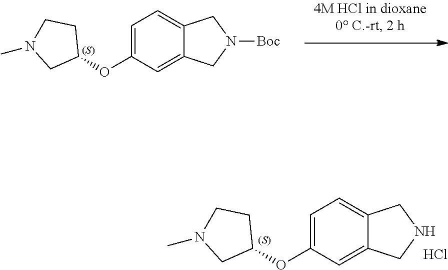

The title compound was prepared from tert-butyl (S)-5-((1-methylpyrrolidin-3-yl)oxy)isoindoline-2-carboxylate (step 1) and 4M HCl in dioxane analogously to Intermediate V step 4.

LCMS (Method N): 1.416 min, MS: ES+ 219.3 (M+1).

### Intermediate VC: 5-((1-Methylazetidin-3-yl)oxy)isoindoline TFA salt

### Step 1: tert-Butyl 5-((1-methylazetidin-3-yl)oxy)isoindoline-2-carboxylate

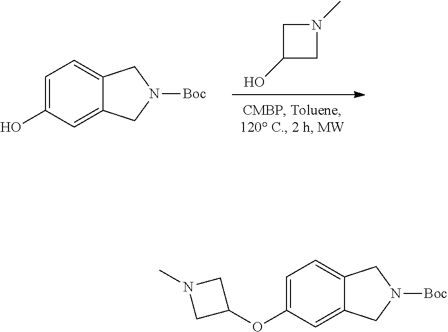

The title compound was prepared from tert-butyl 5-hydroxyisoindoline-2-carboxylate (Intermediate V step 2) and (S)-1-methylpyrrolidin-3-ol (CAS-111043-48-2) analogously to Intermediate V step 3.

¹H NMR (DMSO-d6, 400 MHz): 1H NMR (DMSO-d6, 400 MHz): δ 1.45 (s, 9H), 2.28 (s, 3H), 2.91-2.95 (m, 2H), 3.71-3.75 (m, 2H), 4.47-4.54 (m, 4H), 4.68-4.72 (m, 1H), 6.73-6.79 (m, 2H), 7.19-7.22 (m, 1H).

LCMS (Method N): 1.209 min, MS: ES+ 305.14 (M+1)

### Step 2: 5-((1-Methylazetidin-3-yl)oxy)isoindoline TFA salt

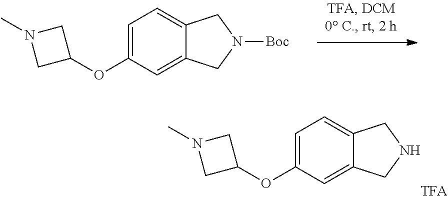 [SMILES: CN1CC(Oc2ccc3c(c2)CN(C(=O)OC(C)(C)C)C3)C1 ; CN1CC(Oc2ccc3c(c2)CNC3)C1]

The title compound was prepared from tert-butyl 5-((1-methylazetidin-3-yl)oxy)isoindoline-2-carboxylate (step 1) and TFA analogously to Intermediate N step 3.

¹H NMR (DMSO-d6, 400 MHz): δ 2.90-2.93 (m, 3H), 4.07 (s, 1H), 4.25 (s, 1H), 4.38-4.50 (m, 5H), 4.73 (s, 1H), 4.97-5.13 (m, 1H), 6.86-6.94 (m, 2H), 7.34-7.37 (m, 1H).

LCMS (Method N): 1.377 min, MS ES+: 205.3 (M+1).

### Intermediate VD: 5-(2-Methoxyethoxy)isoindoline hydrochloride

### Step 1: tert-Butyl 5-(2-methoxyethoxy)isoindoline-2-carboxylate

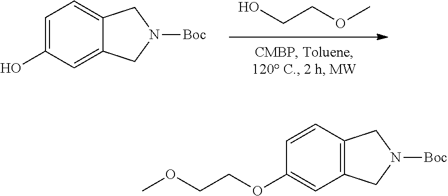

The title compound was prepared from tert-butyl 5-hydroxyisoindoline-2-carboxylate (Intermediate V step 2) and 2-methoxyethan-1-ol (CAS 109-86-4) analogously to Intermediate V step 3.

¹H NMR (DMSO-d6, 400 MHz): δ 1.45 (s, 9H), 3.30 (s, 3H), 3.64 (t, J=4.4 Hz, 4.8 Hz, 2H), 4.05-4.06 (s, 2H), 4.51 (t, J=12 Hz, 4H), 6.84-6.92 (m, 2H), 7.19-7.23 (m, 1H).

LCMS (Method A): 1.783 min, MS: ES+ 237.9 (M−56).

### Step 2: 5-(2-Methoxyethoxy)isoindoline hydrochloride

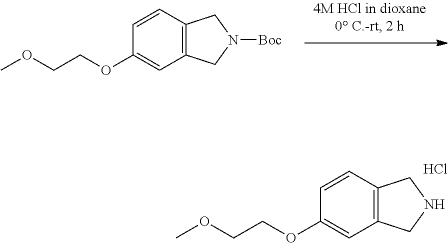 [SMILES: COCCOc1ccc2c(c1)CN(C(=O)OC(C)(C)C)C2 ; Cl ; COCCOc1ccc2c(c1)CNC2]

The title compound was prepared from tert-butyl 5-(2-methoxyethoxy)isoindoline-2-carboxylate (step 1) and 4M HCl in dioxane analogously to Intermediate V step 4.

¹H NMR (DMSO-d6, 400 MHz): δ 3.30 (s, 3H), 3.65 (t, d=4.4 Hz, 2H), 4.09 (t, J=4.4 Hz, 2H), 4.42 (d, J=14.8 Hz, 4H), 6.93 (d, J=8.4 Hz, 1H), 6.99 (s, 1H), 7.29 (d, J=8.4 Hz, 1H), 9.79 (s, 2H).

LCMS (Method A): 0.907 min, MS: ES+ 193.9 (M+1).

### Intermediate VE: 5-((Tetrahydro-2H-pyran-4-yl)oxy)isoindoline hydrochloride

### Step 1: tert-Butyl 5-((tetrahydro-2H-pyran-4-yl)oxy)isoindoline-2-carboxylate

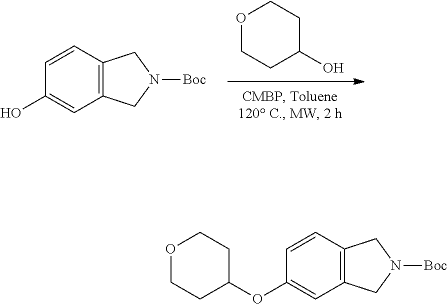 [SMILES: CC(C)(C)OC(=O)N1Cc2ccc(O)cc2C1 ; OC1CCOCC1 ; CC(C)(C)OC(=O)N1Cc2ccc(OC3CCOCC3)cc2C1]

The title compound was prepared from tert-butyl 5-hydroxyisoindoline-2-carboxylate (Intermediate V step 2) and tetrahydro-2H-pyran-4-ol analogously to Intermediate V step 3.

¹H NMR (CDCl₃, 400 MHz): δ 1.53 (s, 9H), 1.77-1.85 (m, 2H), 2.02-2.06 (m, 2H), 3.62 (t, J=4 Hz, 2H), 3.98-4.03 (m, 2H), 4.44-4.51 (m, 1H), 4.64 (t, J=14.8 Hz, 4H), 6.80-6.90 (m, 2H), 7.12-7.20 (dd, J=8 Hz, J=20.4 Hz, 1H).

LCMS (Method N): 1.873 min, MS ES+: 263.9 (M−56).

### Step 2: 5-((Tetrahydro-2H-pyran-4-yl)oxy)isoindoline hydrochloride

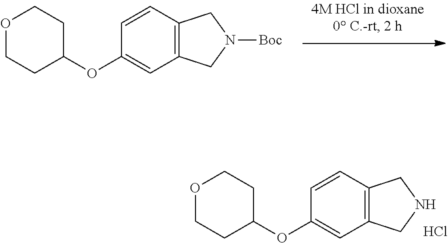 [SMILES: CC(C)(C)OC(=O)N1Cc2ccc(OC3CCOCC3)cc2C1 ; c1cc2c(cc1OC1CCOCC1)CNC2 ; Cl]

The title compound was prepared from [00443] tert-butyl 5-((tetrahydro-2H-pyran-4-yl)oxy)isoindoline-2-carboxylate (step 1) and 4M HCl in dioxane analogously to Intermediate V step 4.

¹H NMR (DMSO-d6, 400 MHz): δ 1.52-1.61 (m, 2H), 1.95-1.99 (m, 2H), 3.49 (t, J=9.6 Hz, 2H), 3.83-3.88 (m, 2H), 4.36-4.44 (m, 4H), 4.53-4.59 (m, 1H), 6.96 (d, J=8.4 Hz, 1H), 7.05 (s, 1H), 7.29 (d, J=8.4 Hz, 1H), 9.75 (bs, 2H).

LCMS (Method N): 1.426 min, MS ES+: 220.29 (M+1).

### Intermediate VF: N,N-Dimethyl-2-((1,2,3,4-tetrahydroisoquinolin-6-yl)oxy)ethan-1-amine hydrochloride

### Step 1: tert-Butyl 6-(4,4,5,5-tetramethyl-1,3,2-dioxaborolan-2-yl)-3,4-dihydroisoquinoline-2(1H)-carboxylate

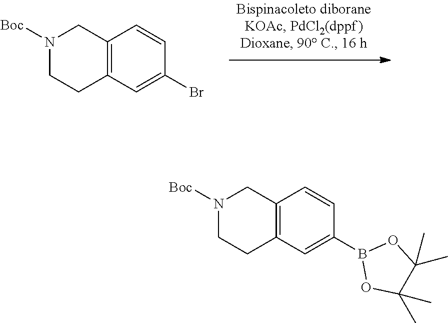 [SMILES: CC(C)(C)OC(=O)N1CCc2cc(Br)ccc2C1 ; CC(C)(C)OC(=O)N1CCc2cc(B3OC(C)(C)C(C)(C)O3)ccc2C1]

The title compound was prepared from tert-butyl 6-bromo-3,4-dihydroisoquinoline-2(1H)-carboxylate (CAS: 893566-74-0) and bis pinacolato diborane analogously to Intermediate V step 1.

¹H NMR (DMSO-d6, 400 MHz): δ 1.28 (s, 12H), 1.42 (s, 9H), 2.78 (t, J=6 Hz, 2H), 3.53 (d, J=6 Hz, 2H), 4.50 (s, 2H), 7.17 (s, 1H), 7.46 (d, J=7.6 Hz, 2H).

LCMS (Method A): 2.981 min, MS ES: 259.8 (M−100).

### Step 2: tert-Butyl 6-hydroxy-3,4-dihydroisoquinoline-2(1H)-carboxylate

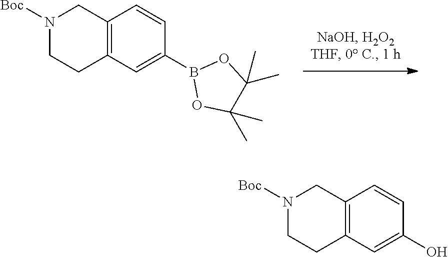

The title compound was prepared from tert-butyl 6-(4,4,5,5-tetramethyl-1,3,2-dioxaborolan-2-yl)-3,4-dihydroisoquinoline-2(1H)-carboxylate (step 1) and NaOH/H₂O₂ analogously to Intermediate V step 2.

¹H NMR (DMSO-d6, 400 MHz): δ 1.42 (s, 9H), 2.67 (t, J=6 Hz, 2H), 3.49 (t, J=6 Hz, 2H), 4.36 (s, 2H), 6.54-6.60 (m, 2H), 6.93 (d, J=8 Hz, 1H), 9.22 (s, 1H).

LCMS (Method A): 2.142 min, MS ES: 194 (M−56).

### Step 3: tert-Butyl 6-(2-(dimethylamino)ethoxy)-3,4-dihydroisoquinoline-2(1H)-carboxylate

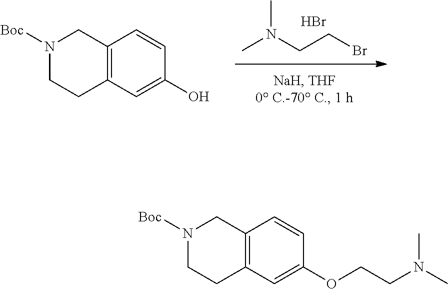

To a stirred solution of tert-butyl 6-hydroxy-3,4-dihydroisoquinoline-2(1H)-carboxylate (step 2) (0.7 g, 2.81 mmol, 1 eq.) in THF (7 mL) at 0° C. was added portionwise NaH (0.33 g, 14.05 mmol, 5.0 eq) and the mixture was stirred for 20 mins. 2-Bromo-N,N-dimethylethan-1-amine (CAS: 2862-39-7) (1.3 g, 5.62 mmol, 2.0 eq) was added dropwise at 0° C. and the reaction mixture was heated to 70° C. for 1 h. The resulting mixture diluted with ice cold water (50 mL) and extracted with EtOAc (3×50 mL). The combined organic extracts were dried over Na₂SO₄, filtered and concentrated under vacuum. The crude material was triturated with n-pentane to yield the title compound (0.6 g, Yield: 72%).

1H NMR (DMSO-d6, 400 MHz): δ 1.42 (s, 9H), 2.19 (s, 6H), 2.58 (t, J=5.6 Hz, 2H), 2.73 (t, J=6 Hz, 2H), 3.51 (t, J=6 Hz, 2H), 4.00 (t, J=5.6 Hz, 2H), 4.40 (s, 2H), 6.75 (d, J=8.8 Hz, 2H), 7.05 (d, J=8 Hz, 1H).

LCMS (Method A): 1.786 min, MS ES+: 321 (M+1).

### Step 4: N,N-Dimethyl-2-((1,2,3,4-tetrahydroisoquinolin-6-yl)oxy)ethan-1-amine hydrochloride

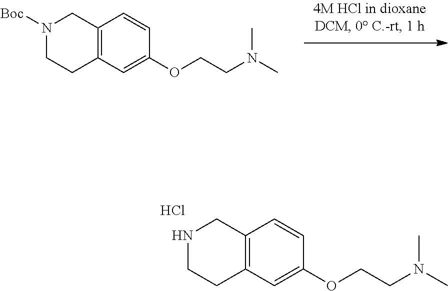 [SMILES: CN(C)CCOc1ccc2c(c1)CCN(C(=O)OC(C)(C)C)C2 ; Cl ; CN(C)CCOc1ccc2c(c1)CCNC2]

The title compound was prepared from tert-butyl 6-(2-(dimethylamino)ethoxy)-3,4-dihydroisoquinoline-2(1H)-carboxylate (step 3) and 4M HCl in dioxane analogously to Intermediate V step 4.

¹H NMR (DMSO-d6, 400 MHz): δ 2.81 (d, J=4.8 Hz, 6H), 2.99 (t, J=6 Hz, 2H), 3.13 (s, 2H), 3.46-3.56 (m, 3H), 4.15 (d, J=4.4 Hz, 2H), 4.35 (t, J=4.8 Hz, 2H), 6.88-6.93 (m, 2H), 7.16 (d, J=8.4 Hz, 1H), 10.84 (s, 1H).

LCMS (Method A): 1.345 min, MS ES+: 221 (M+1).

### Intermediate W: (S)-3-(Isoindolin-4-ylamino)-1-methylpyrrolidin-2-one hydrochloride

### Step 1: (S)-3-Aminopyrrolidin-2-one

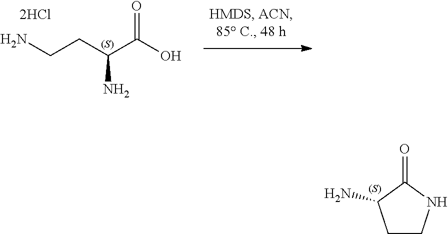

Performed in 2 parallel batches, each of 4.5 g scale: To a stirred solution (S)-2,4-diaminobutanoic acid (CAS: 1883-09-6) (4.5 g, 23.5 mmol, 1 eq.) in acetonitrile (90 mL) was added 1,1,1,3,3,3-hexamethyldisilazane (CAS: 999-97-3) (58.5 mL) and the reaction mixture was heated to 95° C. for 16 h. The resulting mixture was cooled to 0° C., MeOH (25 mL) was added causing a solid to precipitate. The mixture was filtered, and the solids were washed with CHCl₃ (3×200 mL). The filtrate was concentrated under vacuum and the crude material was triturated with n-pentane (2×120 mL) followed by high vacuum drying to yield the title compound as an off white solid (4.3 g, Yield: 91.26%).

¹H NMR (CDCl3, 400 MHz): δ 1.80-1.89 (m, 2H), 2.46-2.53 (m, 1H), 3.27-3.38 (m, 2H), 3.51 (t, J=8.0 Hz, 1H), 6.81 (bs, 1H).

LCMS (Method F): 0.62 min, MS: ES+ 101 (M+1).

### Step 2: (S)-2-(2-Oxopyrrolidin-3-yl) isoindoline-1, 3-dione

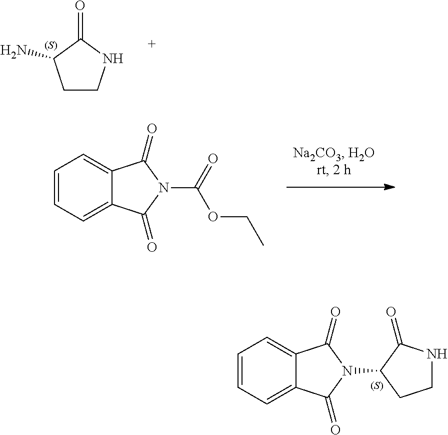

To a stirred solution (S)-3-aminopyrrolidin-2-one_(step 1) (1.7 g, 17.0 mmol, 1 eq.) and Na₂CO₃ (1.07 g, 10.2 mmol, 0.6 eq) in H₂O (17 mL) was added ethyl 1,3-dioxoisoindoline-2-carboxylate (CAS: 22509-74-6) (3.72 g, 17.0 mmol, 1.0 eq) over 10 mins and the reaction mixture was stirred at room temperature for 2 h. The resulting mixture was cooled to 0° C. and acidified to pH 4 with 6N HCl. The precipitated solid was collected by filtration, washed with water (2×100 mL) and dried under vacuum. The crude material was triturated with n-pentane (2×120 mL) and dried under high vacuum to yield the title compound as a white solid (2.3 g, Yield: 91.26%).

¹H NMR (CDCl₃, 400 MHz): δ 2.49-2.65 (m, 2H), 3.47-3.53 (m, 2H), 3.64 (t, J=8.4 Hz, 1H), 4.92-5.00 (m, 1H), 6.37 (bs, 1H), 7.54-7.76 (m, 2H), 7.84-8.00 (m, 2H).

LCMS (Method F): 1.338 min, MS: ES+ 231.25 (M+1).

### Step 3: (S)-2-(1-Methyl-2-oxopyrrolidin-3-yl) isoindoline-1,3-dione

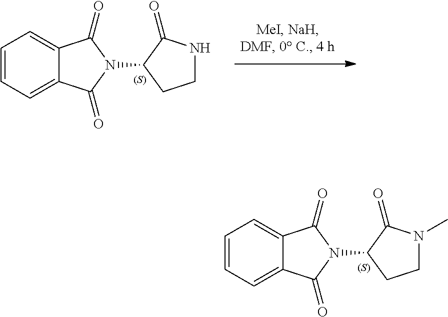

To a stirred solution of (S)-2-(2-oxopyrrolidin-3-yl)isoindoline-1,3-dione (step 2) (2.3 g, 10.0 mmol, 1.0 eq.) in DMF (23 mL) under a nitrogen atmosphere was added dropwise methyl iodide (3.10 g, 22.0 mmol, 2.2 eq.) at room temperature. NaH (60% in mineral oil) (0.520 g, 13.0 mmol, 1.3 eq) was added portion wise to the reaction mixture over 30 mins at 0° C. under a N₂ atmosphere. The reaction mixture was stirred at room temperature for 4 h. The resulting mixture was acidified with acetic acid (2 mL), diluted with ice cold water (50 mL) and extracted with EtOAc (5×50 mL). The combined organic extracts were washed with cold brine solution (3×100 mL), dried over Na₂SO₄, filtered, and concentrated under vacuum. The crude material was purified by chromatography on silica (product eluted at 2.2% MeOH in DCM) to yield the title compound (0.8 g, Yield: 37%).

¹H NMR (CDCl3, 400 MHz): δ 2.37-2.51 (m, 2H), 2.98 (s, 3H), 3.45-3.51 (m, 1H), 3.56-3.62 (m, 1H), 4.97 (t, J=8 Hz, 1H), 7.72-7.76 (m, 2H), 7.84-7.89 (m, 2H).

LCMS (Method A): 1.130 min, MS: ES+ 244.9 (M+1).

### Step 4: (S)-3-Amino-1-methylpyrrolidin-2-one

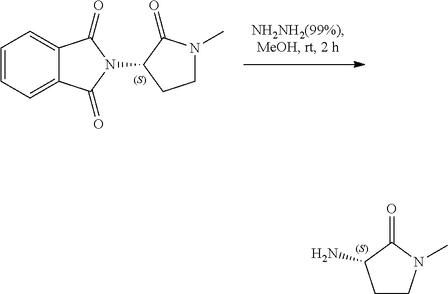

To a stirred solution of (S)-2-(1-methyl-2-oxopyrrolidin-3-yl)isoindoline-1,3-dione (step 3) (0.75 g, 3.07 mmol, 1.0 eq.) in MeOH (7.5 ml) was added dropwise hydrazine hydrate (99%) (3.75 mL) and the mixture was stirred at room temperature for 1.5 h. The resulting mixture was concentrated under vacuum and the crude material was diluted with DCM (5 mL) causing precipitation of a solid. The solid material was removed by filtration and washed with DCM (3×50 mL). The filtrate was dried over Na₂SO₄, filtered, and concentrated under vacuum. The crude material was triturated with n-pentane (20 mL) and dried under high vacuum to yield the title compound as a colourless oil (0.330 g, 3.07 mmol)

¹H NMR (DMSO-d6, 400 MHz): δ 1.55-1.63 (m, 1H), 2.17-2.25 (m, 1H), 2.72 (s, 3H), 2.98 (bs, 3H), 3.18-3.25 (m, 2H), 3.25-3.31 (m, 1H).

LCMS (Method F): 1.05 min, MS ES+: 115.2 (M+1).

### Step 5: tert-Butyl (S)-4-((1-methyl-2-oxopyrrolidin-3-yl)amino)isoindoline-2-carboxylate

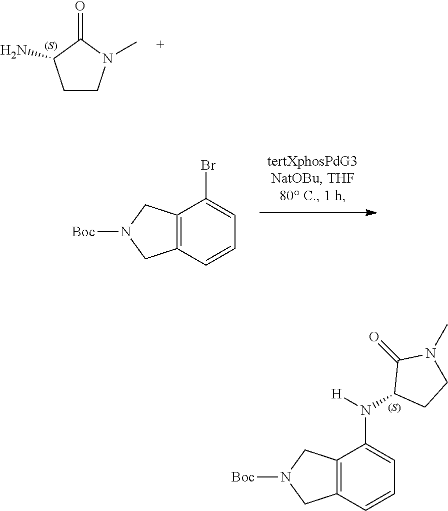

The title compound was prepared from tert-butyl 4-bromoisoindoline-2-carboxylate (Intermediate N step 1) and (S)-3-amino-1-methylpyrrolidin-2-one (step 5) analogously to Intermediate I step 1.

¹H NMR (DMSO-d6, 400 MHz): δ 1.46 (bs, 9H), 1.76-1.82 (m, 1H), 2.40-2.47 (m, 1H), 2.78 (bs, 3H), 3.18 (d, J=12 Hz, 4H), 3.34 (m, 2H), 4.09-4.18 (m, 3H), 4.38-4.42 (m, 2H), 4.52 (d, J=12 Hz, 2H), 5.35-5.40 (m, 1H), 6.56 (d, J=2.8 Hz, 2H), 7.04-7.08 (t, J=8 Hz, 1H).

LCMS (Method A): 1.847 min, MS: ES+ 231.9 (M−100).

### Step 6: (S)-3-(Isoindolin-4-ylamino)-1-methylpyrrolidin-2-one hydrochloride

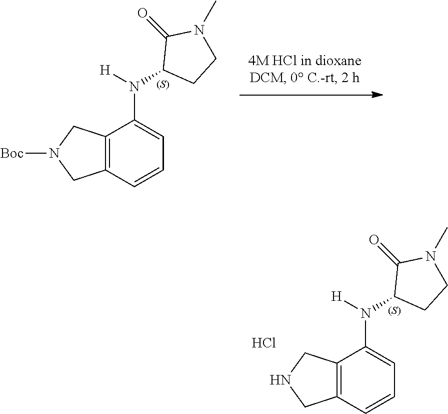

The title compound was prepared from tert-butyl (S)-4-((1-methyl-2-oxopyrrolidin-3-yl)amino)isoindoline-2-carboxylate (step 5) and 4M HCl in dioxane analogously to Intermediate I step 2.

¹H NMR (DMSO-d6, 400 MHz): δ 1.78-1.84 (m, 1H), 2.50-2.51 (m, 1H), 2.78 (s, 3H), 3.10-3.35 (m, 2H), 3.57 (s, 1H), 4.23 (t, J=8 Hz, 1H), 4.31 (t, J=5.2 Hz, 2H), 4.42 (t, J=5.3 Hz, 2H), 6.60-6.65 (m, 2H), 7.13 (t, J=8 Hz, 1H), 9.66 (bs, 2H). HCl salt.

LCMS (Method F): 1.47 min, MS: ES+ 232.3 (M+1).

### Intermediate X: (R)-3-(Isoindolin-4-ylamino)-1-methylpyrrolidin-2-one hydrochloride

### Step 1: (R)-2-(2-Oxopyrrolidin-3-yl)isoindoline-1,3-dione

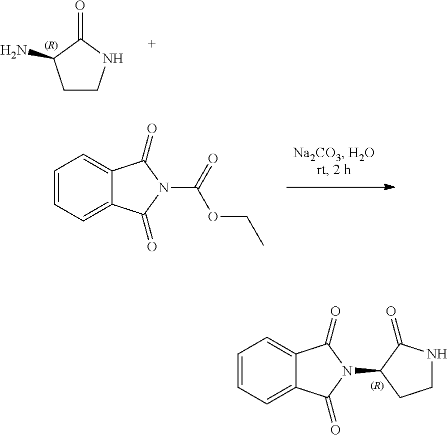

The title compound was prepared from (R)-3-aminopyrrolidin-2-one (CAS: 223407-19-0) and ethyl 1,3-dioxoisoindoline-2-carboxylate (CAS 22509-74-6) analogously to Intermediate W step 2.

¹H NMR (CDCl3, 400 MHz): δ 2.33-2.41 (m, 2H), 3.31-3.38 (m, 2H), 4.81 (t, J=9.6 Hz, 1H), 7.87-7.92 (m, 4H), 8.11 (s, 1H).

LCMS (Method B): 1.341 min, MS: ES+ 231.00 (M+1).

### Step 2: (R)-2-(1-methyl-2-oxopyrrolidin-3-yl) isoindoline-1,3-dione

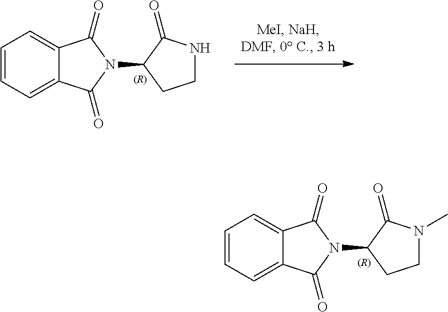

The title compound was prepared from (R)-2-(2-oxopyrrolidin-3-yl)isoindoline-1,3-dione (step 1) and methyl iodide analogously to Intermediate W step 3.

¹H NMR (CDCl3, 400 MHz): δ 2.26-2.35 (m, 2H), 2.80 (s, 3H), 3.38-3.47 (m, 2H), 4.86-4.91 (t, J=9.6 Hz, 1H), 7.89 (bs, 4H).

LCMS (Method A): 1.115 min. MS: ES+ 244.8 (M+1).

### Step 3: (R)-3-Amino-1-methylpyrrolidin-2-one

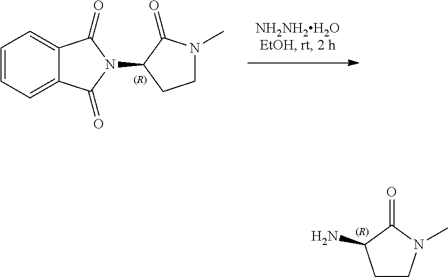

The title compound was prepared from (R)-2-(1-methyl-2-oxopyrrolidin-3-yl)isoindoline-1,3-dione (step 2) and hydrazine hydrate analogously to Intermediate W step 4.

¹H NMR (DMSO-d6, 400 MHz): δ 1.52-1.62 (m, 1H), 1.80 (bs, 1H), 2.14-2.25 (m, 1H), 2.72 (bs, 3H), 3.15-3.20 (m, 2H), 3.22-3.35 (m, 1H).

LCMS (Method F): 0.75 min, MS ES+: 115.2 (M+1).

### Step 4: tert-Butyl (R)-4-((1-methyl-2-oxopyrrolidin-3-yl)amino)isoindoline-2-carboxylate

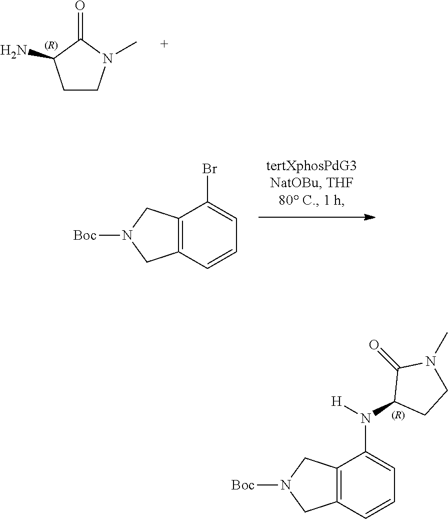

The title compound was prepared from tert-butyl 4-bromoisoindoline-2-carboxylate (Intermediate N step 1) and tert-butyl 4-bromoisoindoline-2-carboxylate (step 3) analogously to Intermediate I step 1.

¹H NMR (DMSO-d6, 400 MHz): δ 1.47 (bs, 9H), 1.76-1.85 (m, 1H), 2.41-2.51 (m, 1H), 2.75-2.78 (m, 3H), 3.26-3.36 (m, 2H), 4.20 (bs, 1H), 4.34-4.44 (m, 2H), 4.46-4.53 (m, 2H), 5.37-5.42 (m, 1H), 6.54-6.57 (m, 2H), 7.04-7.08 (m, 1H).

LCMS (Method A): 1.791 min, MS: ES+ 232 (M−100).

### Step 5: (R)-3-(Isoindolin-4-ylamino)-1-methylpyrrolidin-2-one hydrochloride (analogous to Int W step 6)

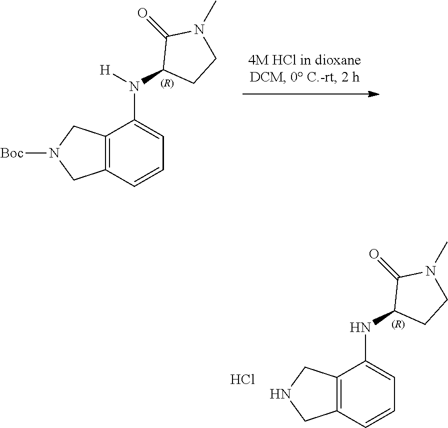

The title compound was prepared from tert-butyl (R)-4-((1-methyl-2-oxopyrrolidin-3-yl)amino)isoindoline-2-carboxylate (step 4) and 4M HCl in dioxane analogously to Intermediate I step 2.

¹H NMR (DMSO-d6, 400 MHz): δ 1.77-1.87 (m, 1H), 2.40-2.51 (m, 1H), 2.78 (bs, 3H), 3.28-3.38 (m, 2H), 3.56 (s, 2H), 4.21 (t, J=8.8 Hz, 3H), 4.29-4.42 (m, 2H), 5.77 (s, 2H), 6.60-6.65 (m, 2H), 7.13 (t, J=7.6 Hz, 1H), 6.51 (d, J=7.6 Hz, 1H), 6.63 (d, J=8 Hz, 1H), 7.06 (t, J=8 Hz, 1H), 9.98 (bs, 2H) HCl salt.

LCMS (Method F): 1.44 min, MS: ES+ 232.3 (M+1).

### Intermediate YA: N,N-Dimethyl-2-((1,2,3,4-tetrahydroisoquinolin-7-yl)oxy)ethan-1-amine hydrochloride

### Step 1: tert-Butyl 7-(4,4,5,5-tetramethyl-1,3,2-dioxaborolan-2-yl)-3,4-dihydroisoquinoline-2(1H)-carboxylate

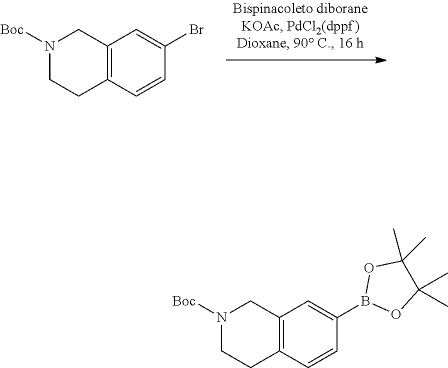 [SMILES: CC(C)(C)OC(=O)N1CCc2ccc(Br)cc2C1 ; CC(C)(C)OC(=O)N1CCc2ccc(B3OC(C)(C)C(C)(C)O3)cc2C1]

To a stirred solution of tert-butyl 7-bromo-3,4-dihydroisoquinoline-2(1H)-carboxylate (5 g, 16.02 mmol, 1 eq) (CAS: 258515-65-0) in 1-4 dioxane (50 mL) were added Bispinacolato diborane (4.86 g, 19.23 mmol, 1.2 eq) and potassium acetate (3.14 g, 32.05 mmol, 2.0 eq) at room temperature. The mixture was purged with N₂ for 30 min. PdCl₂(dppf) (2.34 g, 3.20 mmol, 0.2 eq) was added and the reaction mixture was heated to 90° C. for 16 h. The resulting mixture was diluted with water (100 mL) and extracted with EtOAc (3×100 mL). The combined organic extracts were dried over Na₂SO₄, filtered and concentrated under vacuum. The crude material was purified by chromatography on silica (eluting with 9% EtOAc in hexane) yielding tert-butyl 7-(4,4,5,5-tetramethyl-1,3,2-dioxaborolan-2-yl)-3,4-dihydroisoquinoline-2(1H)-carboxylate (4 g, Yield: 78%).

¹H NMR (CDCl3, 400 MHz): δ 1.36 (s, 12H), 1.50 (s, 9H), 2.87 (bs, 2H), 3.66 (bs, 2H), 4.60 (s, 2H), 7.17 (d, J=7.6 Hz, 1H), 7.61 (t, J=7.6 Hz, 2H).

LCMS (Method A): 2.641 min, MS ES−: 259.8 (M−100).

### Step 2: tert-Butyl 7-hydroxy-3,4-dihydroisoquinoline-2(1H)-carboxylate

To a stirred solution of tert-butyl 7-(4,4,5,5-tetramethyl-1,3,2-dioxaborolan-2-yl)-3,4-dihydroisoquinoline-2(1H)-carboxylate (step 1) (4.0 g, 11.14 mmol, 1 eq.) in THE (40 ml) was added NaOH (0.89 g, 22.28 mmol, 2.0 eq) at 0° C. followed by dropwise addition of H₂O₂ (30% in water) (5.30 g, 46.79 mmol, 4.2 eq). The reaction mixture was allowed to stir at 0° C. for 1 h. The resulting mixture was diluted with water (100 mL) and extracted with EtOAc (3×50 mL). The combined organic extracts were dried over Na₂SO₄, filtered and concentrated under vacuum. The crude material was purified by chromatography on silica (product eluted at 4% MeOH in DCM) yielding tert-butyl 7-hydroxy-3,4-dihydroisoquinoline-2(1H)-carboxylate (3.0 g, Yield: 96%).

¹H NMR (DMSO-d⁶, 400 MHz): δ 1.42 (s, 9H), 2.64 (t, J=5.6 Hz, 2H), 3.50 (t, J=6 Hz, 2H), 4.39 (bs, 2H), 6.52 (bs, 1H), 6.57 (d, J=8.4 Hz, 1H), 6.93 (d, J=8.4 Hz, 1H), 9.23 (s, 1H).

LCMS (Method A): 1.763 min, MS ES−: 193.88 (M−56).

### Step 3: tert-Butyl 7-(2-(dimethylamino)ethoxy)-3,4-dihydroisoquinoline-2(1H)-carboxylate

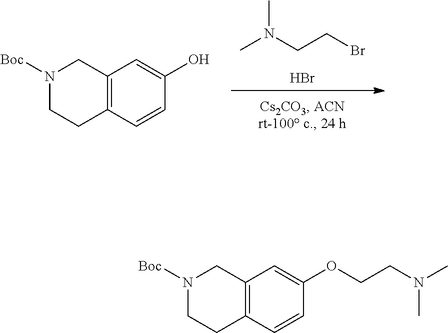

To a stirred solution of tert-butyl 7-hydroxy-3,4-dihydroisoquinoline-2(1H)-carboxylate (step 2) (0.5 g, 20.00 mmol, 1 eq.) in acetonitrile (5 mL) were added CS₂CO₃ (1.7 g, 52.00 mmol, 2.6 eq) and 2-bromo-N,N-dimethylethan-1-amine HBr (CAS: 2862-39-7) (0.559 g, 24.00 mmol, 1.2 eq) at room temperature. The reaction mixture was heated to 100° C. temperature for 16 h. The resulting mixture was diluted with water (50 mL) and extracted with EtOAc (3×20 mL). The combined organic extracts were dried over Na₂SO₄, filtered and concentrated under vacuum. The crude material was purified by chromatography on silica (product eluted at 6% MeOH in DCM) yielding tert-butyl 7-(2-(dimethylamino)ethoxy)-3,4-dihydroisoquinoline-2(1H)-carboxylate (0.150 g, Yield: 23%).

¹H NMR (CDCl3, 400 MHz): δ 1.50 (s, 9H), 2.29 (s, 6H), 2.78 (t, J=5.2 Hz, 4H), 3.64 (bs, 2H), 4.09 (t, J=5.6 Hz, 2H), 4.55 (s, 2H), 6.68 (bs, 1H), 6.78-6.76 (dd, J=2.4 Hz, J=2.8 Hz, 1H), 7.05 (d, J=8.4 Hz, 1H).

LCMS (Method D): 2.95 min, MS ES+ 321 (M+1).

### Step 4: N,N-Dimethyl-2-((1,2,3,4-tetrahydroisoquinolin-7-yl)oxy)ethan-1-amine hydrochloride

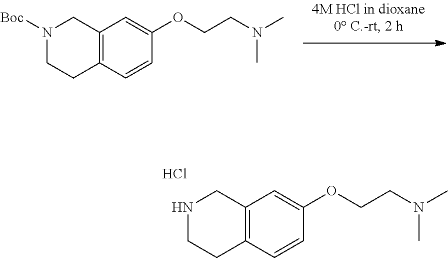 [SMILES: CN(C)CCOc1ccc2c(c1)CN(C(=O)OC(C)(C)C)CC2 ; Cl ; CN(C)CCOc1ccc2c(c1)CNCC2]

To a stirred solution of tert-butyl 7-(2-(dimethylamino)ethoxy)-3,4-dihydroisoquinoline-2(1H)-carboxylate (0.150 g, 0.46 mmol, 1.0 eq.) in DCM (1.5 mL) was added dropwise 4M HCl in dioxane (1.5 mL) at 0° C. The reaction mixture was allowed to stir at room temperature for 2 h. The resulting mixture was concentrated under vacuum. The crude material was triturated with diethyl ether (2×5 mL) followed by high vacuum drying yielding N,N-dimethyl-2-((1,2,3,4-tetrahydroisoquinolin-7-yl)oxy)ethan-1-amine hydrochloride (0.090 g, Yield: 74%).

¹H NMR (DMSO-d6, 400 MHz): δ 2.81 (d, J=4.8 Hz, 6H), 2.99 (t, J=6 Hz, 2H), 3.25 (bs, 2H), 3.46-3.54 (m, 2H), 3.57 (s, 1H), 4.15 (d, J=4 Hz, 2H), 4.35 (t, J=4.8 Hz, 2H), 6.88-6.94 (m, 2H), 7.17 (d, J=8.4 Hz, 1H), 9.64 (bs, 2H), 10.84 (bs, 1H).

LCMS (Method Q): 1.345 min, MS ES+: 221 (M+1).

### Intermediate YB: N,N-Dimethyl-1-(1,2,3,4-tetrahydroisoquinolin-7-yl)methanamine

### Step 1: 2-(tert-Butyl) 7-methyl 3,4-dihydroisoquinoline-2,7(1H)-dicarboxylate

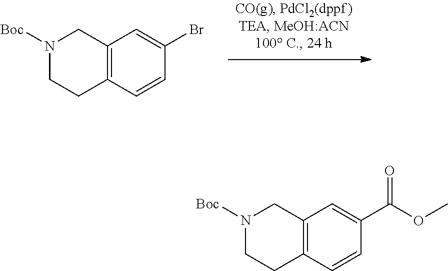 [SMILES: CC(C)(C)OC(=O)N1CCc2ccc(Br)cc2C1 ; COC(=O)c1ccc2c(c1)CN(C(=O)OC(C)(C)C)CC2]

To a stirred solution of tert-butyl 7-bromo-3,4-dihydroisoquinoline-2(1H)-carboxylate (5.0 g, 16.0 mmol, 1.0 eq) in MeOH (66 mL) and acetonitrile (16 mL) in an autoclave was added TEA (3.23 g, 3.20 mmol, 2.0 eq) at room temperature. The mixture was degassed under nitrogen gas for 15 min. PdCl₂(dppf) (0.93 g, 1.28 mmol, 0.08 eq) was added and the mixture was heated to 110° C. under a pressure of 25 kg/cm² CO gas for 16 h. The reaction mixture was concentrated under vacuum. The crude material was purified by chromatography on silica eluting with 2.5% EtOAc in hexane to yield 2-(tert-butyl) 7-methyl 3,4-dihydroisoquinoline-2,7(1H)-dicarboxylate (3.5 g, Yield: 75%).

¹H NMR (DMSO-d6, 400 MHz): δ 1.43 (s, 9H), 2.84 (t, J=6 Hz, 2H), 3.56 (t, J=5.6 Hz, 2H), 3.81 (s, 3H), 4.56 (s, 2H), 7.31 (d, J=8 Hz, 1H), 7.74 (s, 1H), 7.76 (m, 1H).

LCMS (Method A): 2.111 min, MS: ES+ 192.18 (M−100).

### Step 2: 2-(tert-Butoxycarbonyl)-1,2,3,4-tetrahydroisoquinoline-7-carboxylic acid

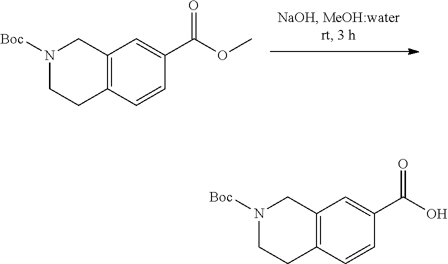 [SMILES: COC(=O)c1ccc2c(c1)CN(C(=O)OC(C)(C)C)CC2 ; CC(C)(C)OC(=O)N1CCc2ccc(C(=O)O)cc2C1]

To a stirred solution of 2-(tert-butyl) 7-methyl 3,4-dihydroisoquinoline-2,7(1H)-dicarboxylate (step 1) (3.5 g, 12.02 mmol, 1.0 eq.) in MeOH:water (9:1) (50 mL) was added NaOH (2.40 g, 60.1 mmol, 5.0 eq.) and the mixture was stirred at room temperature for 3 h. The resulting mixture was poured into water (60 mL) and the pH was adjusted to pH1-2 with dilute HCl solution. The mixture was extracted with EtOAc (3×90 mL) and the combined organic extracts were dried over Na₂SO₄, filtered and concentrated under vacuum. The crude material was purified by trituration with n-pentane (250 mL) yielding 2-(tert-butoxycarbonyl)-1,2,3,4-tetrahydroisoquinoline-7-carboxylic acid brown oil (3.5 g, Yield: Quantitative, 12.63 mmol).

1H NMR (DMSO-d6, 400 MHz): δ 1.42 (s, 9H), 2.75 (t, J=5.6 Hz, 2H), 3.52-3.55 (m, 2H), 4.48 (bs, 2H), 7.05 (d, J=7.6 Hz, 1H), 7.66-7.694 (m, 2H). —COOH proton not observed.

LCMS (Method A): 1.783 min, MS: ES+ 178.17 (M−100)

### Step 3: tert-Butyl 7-(dimethylcarbamoyl)-3,4-dihydroisoquinoline-2(1H)-carboxylate

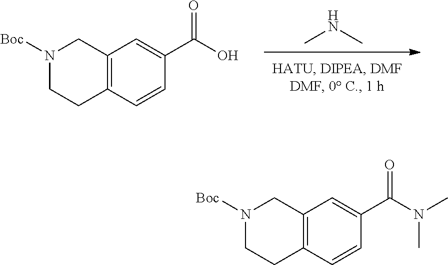

To a stirred solution of 2-(tert-butoxycarbonyl)-1,2,3,4-tetrahydroisoquinoline-7-carboxylic acid (step 2) (2.5 g, 9.2 mmol, 1.0 eq) in DMF (25 mL) were added HATU (5.14 g, 13.53 mmol, 1.5 eq) and DIPEA (2.32 g, 18.04 mmol, 2.0 eq) at 0° C. Dimethyl amine (2M in THF) (0.44 g, 99.23 mmol, 1.1 eq.) was added dropwise to the reaction mixture at 0-5° C. The mixture was allowed to warm to room temperature and stirred for 1 h. The resulting mixture was diluted with EtOAc (45 mL) and washed with cold brine solution (4×50 mL). The combined organic extracts were dried over Na₂SO₄, filtered and concentrated under vacuum. The crude material was purified by chromatography on silica (product eluted at 4% methanol in DCM) yielding tert-butyl 7-(dimethylcarbamoyl)-3,4-dihydroisoquinoline-2(1H)-carboxylate as an off-white solid (2.1 g, Yield: 76%).

¹H NMR (DMSO-d6, 400 MHz): 1H NMR (DMSO-d6, 400 MHz): δ 1.44 (s, 9H), 2.80 (t, J=6 Hz, 2H), 2.95 (d, J=19.2 Hz, 6H), 3.57 (t, J=6 Hz, 2H), 4.52 (s, 2H), 7.21-7.22 (m, 3H).

LCMS (Method A): 1.687 min, MS: ES+ 249.05 (M−56).

### Step 4: N,N-Dimethyl-1,2,3,4-tetrahydroisoquinoline-7-carboxamide

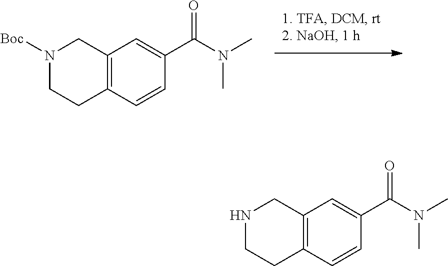 [SMILES: CN(C)C(=O)c1ccc2c(c1)CN(C(=O)OC(C)(C)C)CC2 ; CN(C)C(=O)c1ccc2c(c1)CNCC2]

To a stirred solution of tert-butyl 7-(dimethylcarbamoyl)-3,4-dihydroisoquinoline-2(1H)-carboxylate (step 3) (2.1 g, 6.90 mmol, 1.0 eq.) in DCM (21 mL) was added dropwise TFA (2.1 mL) at 0° C. The reaction mixture was stirred at room temperature for 1 h then concentrated under vacuum. The residue was poured into water (60 mL) and the pH was adjusted to pH 9-10 with 1M NaOH solution. The mixture was extracted with DCM (5×60 mL) and the combined organic extracts were dried over Na₂SO₄, filtered and concentrated under vacuum yielding N,N-dimethyl-1,2,3,4-tetrahydroisoquinoline-7-carboxamide as an oil (1.8 g, 8.81 mmol, Yield: 78%). LCMS (Method B): 0.463 min, MS: ES+ 205.25 (M+1).

### Step 5: N,N-Dimethyl-1-(1,2,3,4-tetrahydroisoquinolin-7-yl) methenamine

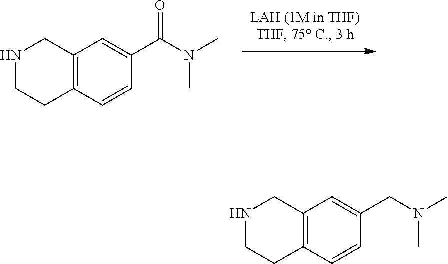

To a stirred solution of N, N-dimethyl-1,2,3,4-tetrahydroisoquinoline-7-carboxamide (step 4) (0.2 g, 0.97 mmol, 1.0 eq.) in THF (2 mL) was added dropwise LAH (1M in THF) (2.84 mL, 2.9 eq) at 0° C. The resulting mixture was heated to 80° C. and stirred for 2 h. The resulting mixture was allowed to cool room temperature and diluted with water (10 mL). The pH was adjusted to pH 10-12 with 1M NaOH solution and the mixture was extracted with EtOAc (3×25 mL). The combined organic extracts were dried over Na₂SO₄, filtered and concentrated under vacuum yielding N, N-dimethyl-1-(1,2,3,4-tetrahydroisoquinolin-7-yl) methenamine as yellow oil (0.18 g, 0.94 mmol, Yield: 97%).

LCMS (Method B): 1.52 min, MS: ES+ 191.28 (M+1).

### Intermediate YC: N,N-Dimethyl-1-(1,2,3,4-tetrahydroisoquinolin-6-yl)methanamine

### Step 1: 2-(tert-Butyl) 6-methyl 3,4-dihydroisoquinoline-2,6(1H)-dicarboxylate

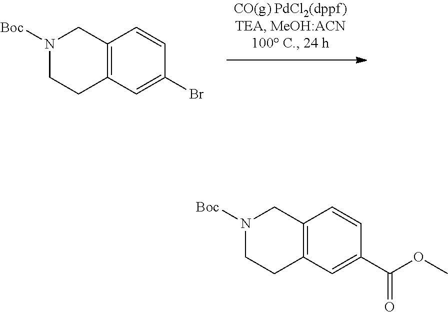 [SMILES: CC(C)(C)OC(=O)N1CCc2cc(Br)ccc2C1 ; COC(=O)c1ccc2c(c1)CCN(C(=O)OC(C)(C)C)C2]

The title compound was prepared from tert-butyl 6-bromo-3,4-dihydroisoquinoline-2(1H)-carboxylate, PdCl₂(dppf) and CO gas analogously to Intermediate YB step 1.

¹H NMR (DMSO-d6, 400 MHz): δ 1.43 (s, 9H), 2.85 (t, J=6.0 Hz, 2H), 3.57 (t, J=6.0 Hz, 2H), 3.84 (s, 3H), 4.57 (s, 2H), 7.32 (d, J=8.0 Hz, 1H), 7.76 (d, J=8.0 Hz, 2H).

LCMS (Method A): 2.233 min, MS: ES+ 192.18 (M+1).

### Step 2: 2-(tert-Butoxycarbonyl)-1,2,3,4-tetrahydroisoquinoline-6-carboxylic acid

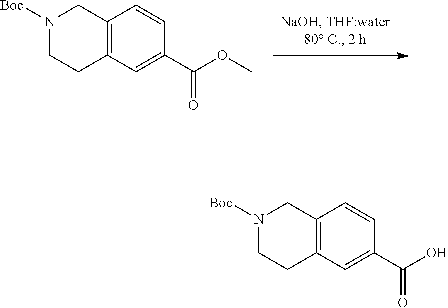 [SMILES: COC(=O)c1ccc2c(c1)CCN(C(=O)OC(C)(C)C)C2 ; CC(C)(C)OC(=O)N1CCc2cc(C(=O)O)ccc2C1]

The title compound was prepared from 2-(tert-butyl) 6-methyl 3,4-dihydroisoquinoline-2,6(1H)-dicarboxylate (step 1) and NaOH analogously to Intermediate YB step 2.

¹H NMR (DMSO-d6, 400 MHz): δ 1.43 (s, 9H), 2.84 (t, J=5.6 Hz, 2H), 3.57 (t, J=5.6 Hz, 2H), 4.56 (s, 2H), 7.29 (d, J=8.0 Hz, 1H), 7.74 (d, J=7.6 Hz, 1H), 12.90 (s, 1H).

LCMS (Method A): 1.837 min, MS: ES, 178.17 (M−100).

### Step 3: tert-Butyl 6-(dimethylcarbamoyl)-3,4-dihydroisoquinoline-2(1H)-carboxylate

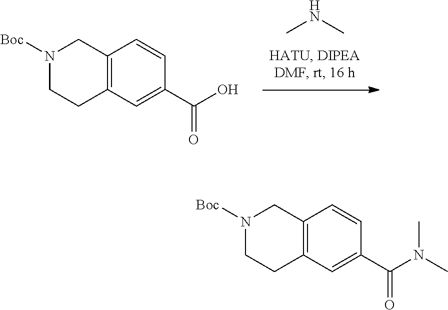

The title compound was prepared from 2-(tert-butoxycarbonyl)-1,2,3,4-tetrahydroisoquinoline-6-carboxylic acid (step 2) and dimethyl amine analogously to Intermediate YB step 3.

¹H NMR (DMSO-d6, 400 MHz): δ 1.43 (s, 9H), 2.79 (t, J=6.0 Hz, 2H), 2.96-2.99 (s, 6H), 3.56 (t, J=6.0 Hz, 2H), 4.52 (s, 2H), 7.20-7.24 (m, 3H).

LCMS (Method A): 1.775 min, MS: ES+ 249.05 (M−56).

### Step 4: N, N-Dimethyl-1,2,3,4-tetrahydroisoquinoline-6-carboxamide

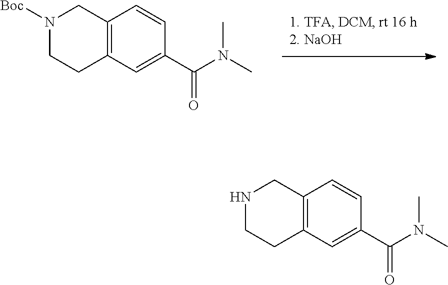 [SMILES: CN(C)C(=O)c1ccc2c(c1)CCN(C(=O)OC(C)(C)C)C2 ; CN(C)C(=O)c1ccc2c(c1)CCNC2]

The title compound was prepared from tert-butyl 6-(dimethyl carbamoyl)-3,4-dihydroisoquinoline-2(1H)-carboxylate (step 3) and TFA analogously to Intermediate YB step 4.

¹H NMR (DMSO-d6, 400 MHz): δ 2.70 (t, J=5.2 Hz, 2H), 2.91-2.96 (m, 8H), 3.87 (s, 2H), 7.04-7.13 (m, 3H).

LCMS (Method B): 1.40 min, MS: ES+ 205.25 (M+1).

### Step 5: N,N-Dimethyl-1-(1,2,3,4-tetrahydroisoquinolin-6-yl) methanamine

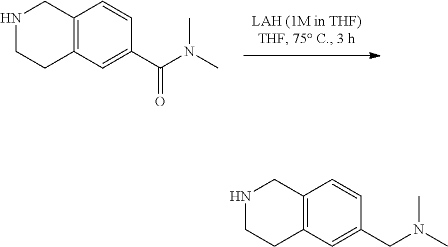

The title compound was prepared from N, N-dimethyl-1,2,3,4-tetrahydroisoquinoline-6-carboxamide (step 4) and LAH (2M in THF) analogously to Intermediate YB step 5

¹H NMR (DMSO-d6, 400 MHz): δ 2.09-2.11 (m, 6H), 2.64-2.65 (m, 2H), 2.90-2.91 (m, 2H), 3.27-3.29 (m, 2H), 3.78-3.80 (m, 2H), 6.93-6.99 (m, 1H).

LCMS (Method B): 1.39 min, MS: ES+ 191.28 (M+1).

### Intermediate YD: 7-((1-Methylpiperidin-4-yl)oxy)-1,2,3,4-tetrahydroisoquinoline hydrochloride

### Step 1: tert-Butyl 7-((1-methylpiperidin-4-yl)oxy)-3,4-dihydroisoquinoline-2(1H)-carboxylate

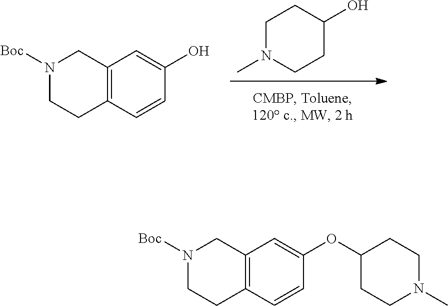

To a stirred solution of tert-butyl 7-hydroxy-3,4-dihydroisoquinoline-2(1H)-carboxylate (Intermediate YA step 2) (0.22 g, 0.88 mmol, 1 eq), 1-m ethylpiperidin-4-ol (0.152 g, 1.30 mmol, 1.5 eq) (CAS: 106-52-5) in toluene (2.2 mL) was added (tributylphosphoranylidene)acetonitrile solution (1M in toluene) (CMBP) (0.425 g, 1.76 mmol, 2 eq) at room temperature. The reaction mixture was heated to 120° C. using microwave radiation for 2 h. The resulting mixture was concentrated under vacuum and purification of the crude material by chromatography on silica, (eluting with 4.5% MeOH in DCM) yielding tert-butyl 7-((1-methylpiperidin-4-yl)oxy)-3,4-dihydroisoquinoline-2(1H)-carboxylate (0.110 g, Yield: 36%).

¹H NMR (DMSO-d6, 400 MHz): δ 1.43 (s, 9H), 1.60-1.69 (m, 3H), 1.89-1.91 (m, 2H), 2.22 (bs, 3H), 2.67 (t, J=4.4 Hz, 3H), 3.52 (t, J=5.2 Hz, 2H), 4.43 (bs, 1H), 4.45 (bs, 2H), 6.76 (t, J=8.4 Hz, 2H), 7.04 (d, J=8.4 Hz, 1H).

LCMS (Method A): 1.474 min, MS ES+: 347.1 (M+1).

### Step 2: 7-((1-Methylpiperidin-4-yl)oxy)-1,2,3,4-tetrahydroisoquinoline hydrochloride

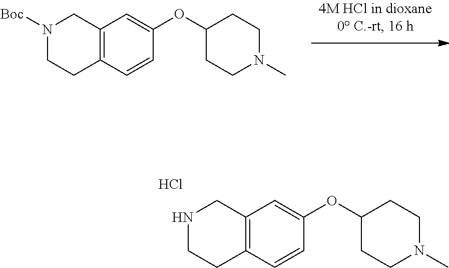 [SMILES: CN1CCC(Oc2ccc3c(c2)CN(C(=O)OC(C)(C)C)CC3)CC1 ; Cl ; CN1CCC(Oc2ccc3c(c2)CNCC3)CC1]

To a stirred solution of tert-butyl 7-((1-methylpiperidin-4-yl)oxy-3,4-dihydroisoquinoline-2(1H)-carboxylateas (step 1)(0.110 g, 0.32 mmol, 1 eq) in DCM (1.1 mL), cooled to 0° C., was added dropwise 4M HCl in dioxane (1.1 mL) and the mixture was stirred at room temperature for 16 h. The resulting mixture was concentrated under vacuum and the crude material was triturated with n-pentane (10 mL) followed by high vacuum drying to yield 7-((1-methylpiperidin-4-yl)oxy)-1,2,3,4-tetrahydroisoquinoline hydrochloride as off-white solid (0.090 g, Yield: 100%).

¹H NMR (DMSO-d6, 400 MHz): δ 1.84-1.90 (m, 1H), 1.93-1.98 (m, 1H), 2.02-2.21 (m, 2H), 2.73-2.76 (m, 3H), 2.93 (bs, 2H), 3.01-3.15 (m, 2H), 3.25-3.33 (m, 2H), 3.45 (d, J=11.2 Hz, 1H), 3.65 (bs, 4H), 4.19 (bs, 2H), 4.70 (bs, 1H), 6.90-6.97 (m, 2H), 7.15 (t, J=8.4 Hz, 1H), 10.87 (bs, 1H).

LCMS (Method N): 1.717 min, MS ES+: 247.27 (M+1).

### Intermediate YE: 6-((1-Methylpiperidin-4-yl)oxy)-1,2,3,4-tetrahydroisoquinoline hydrochloride

### Step 1: tert-Butyl 6-((1-methylpiperidin-4-yl)oxy)-3,4-dihydroisoquinoline-2(1H)-carboxylate

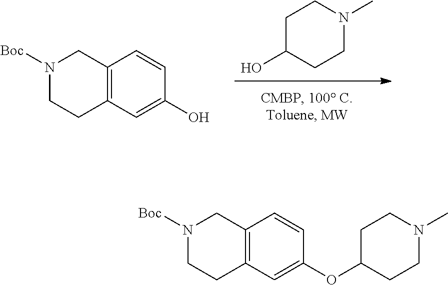

The title compound was prepared from 1-methylpiperidin-4-ol and tert-butyl 6-hydroxy-3,4-dihydroisoquinoline-2(1H)-carboxylate (Intermediate VF step 2) analogously to Intermediate YD step 1.

¹H NMR (DMSO-d6, 400 MHz): δ 0.90-0.94 (m, 1H), 1.24-1.25 (m, 2H), 1.42 (s, 9H), 1.60 (d, J=8 Hz, 2H), 1.89 (d, J=12 Hz, 2H), 2.18 (s, 4H), 2.72 (t, J=8 Hz, 12 Hz, 2H), 3.50 (t, J=8 Hz, 12 Hz, 2H), 4.31 (bs, 1H), 4.40 (s, 1H), 6.74-6.78 (m, 2H), 7.04 (d, J=12 Hz, 1H),

LCMS (Method A): 1.420 min, MS: ES+ 347.2 (M+1).

### Step 2: 6-((1-Methylpiperidin-4-yl)oxy)-1,2,3,4-tetrahydroisoquinoline hydrochloride

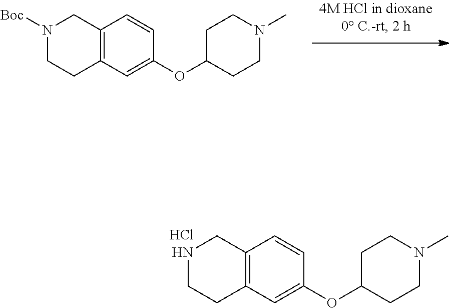 [SMILES: CN1CCC(Oc2ccc3c(c2)CCN(C(=O)OC(C)(C)C)C3)CC1 ; Cl ; CN1CCC(Oc2ccc3c(c2)CCNC3)CC1]

The title compound was prepared from tert-butyl 6-((1-methyl piperidin-4-yl)oxy)-3,4-dihydroisoquinoline-2(1H)-carboxylate (step 1) and 4M HCl in dioxane analogously to Intermediate YD step 1.

LCMS (Method N): 0.249 min, MS: ES+ 247.2 (M+1).

### Intermediate YF: 7-((4-Methyl piperazin-1-yl)methyl)-1,2,3,4-tetrahydroisoquinoline hydrochloride

### Step 1: tert-Butyl 7-(hydroxymethyl)-3,4-dihydroisoquinoline-2(1H)-carboxylate

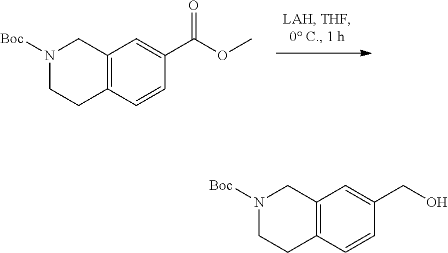 [SMILES: COC(=O)c1ccc2c(c1)CN(C(=O)OC(C)(C)C)CC2 ; CC(C)(C)OC(=O)N1CCc2ccc(CO)cc2C1]

To a stirred solution of 2-(tert-butyl) 7-methyl 3,4-dihydroisoquinoline-2,7(1H)-dicarboxylate (Intermediate YB step 1) (5 g, 17.18 mmol, 1 eq) in THF (50 mL) was added dropwise LAH solution (1M THF) (10.30 mL, 10.30 mmol, 0.6 eq) and the reaction mixture was stirred at 0° C. for 1 h. The resulting mixture was diluted with water (30 mL), quenched with saturated NH₄Cl solution (50 mL) and extracted with EtOAc (3×150 mL). The combined organic extracts were dried over Na₂SO₄, filtered, and concentrated under vacuum. The crude material was purified by trituration with n-pentane (3×50 mL) followed by high vacuum drying yielding tert-butyl 7-(hydroxymethyl)-3,4-dihydroisoquinoline-2(1H)-carboxylate (4.3 g, Yield: 88.85%).

¹H NMR (DMSO-d6, 400 MHz): δ 1.36-1.45 (m, 9H), 2.74 (t, J=6 Hz, 2H), 3.53 (t, J=6 Hz, 2H), 4.45 (t, J=5.6 Hz, 4H), 5.13 (t, J=6 Hz, 1H), 7.09 (d, J=6.8 Hz, 3H).

LCMS (Method A): 1.697 min, MS: ES+ 163.8 (M−100).

### Step 2: tert-Butyl 7-(bromomethyl)-3,4-dihydroisoquinoline-2(1H)-carboxylate

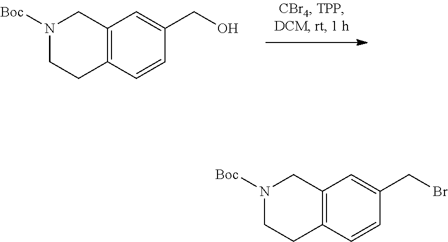 [SMILES: CC(C)(C)OC(=O)N1CCc2ccc(CO)cc2C1 ; CC(C)(C)OC(=O)N1CCc2ccc(CBr)cc2C1]

The title compound was prepared from tert-butyl 7-(hydroxymethyl)-3,4-dihydroisoquinoline-2(1H)-carboxylate (step 1), CBr₄ and triphenyl phosphine analogously to Intermediate R step 3.

¹H NMR (DMSO-d6, 400 MHz): δ 1.43 (s, 9H), 2.49-2.51 (m, 2H), 3.54 (t, J=6 Hz, 2H), 4.48 (s, 2H), 4.67 (s, 2H), 7.15 (d, J=8.4 Hz, 1H), 7.25 (d, J=2 Hz, 2H).

LCMS (Method A): 2.339 min, MS: ES− 269.7 (M−56)

### Step 3: tert-Butyl 7-((4-methylpiperazin-1-yl)methyl)-3,4-dihydroisoquinoline-2(1H)-carboxylate

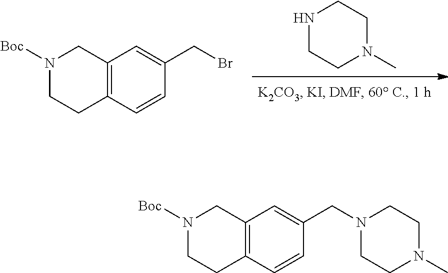

To a stirred solution of 1-methylpiperazine (0.36 g, 3.68 mmol, 1.5 eq.) (CAS: 109 01-3) was added K₂CO₃ (1.01 g, 7.36 mmol, 3.0 eq) and the mixture was stirred at room temperature for 20 mins. tert-Butyl 7-(hydroxymethyl)-3,4-dihydroisoquinoline-2(1H)-carboxylate (step 2) (0.8 g, 2.45 mmol, 1.0 eq) and KI (0.04 g, 0.24 mmol, 0.1 eq) were added and the mixture was heated to 60° C. for 1 h. The resulting mixture was diluted with ice cold water (50 mL) and extracted with EtOAc (3×30 mL). The combined organic extracts were dried over Na₂SO₄, filtered and concentrated under vacuum. The crude material was purified by chromatography on silica (eluting with 4.5% methanol in DCM) yielding tert-butyl 7-((4-methyl piperazin-1-yl)methyl)-3,4-dihydroisoquinoline-2(1H)-carboxylate (0.6 g, Yield: 70.9%).

¹H NMR (DMSO-d6, 400 MHz): δ 1.42 (s, 9H), 2.19 (s, 3H), 2.26-2.50 (m, 6H), 2.74 (t, J=6 Hz 2H), 3.34-3.41 (m, 2H), 3.53 (t, J=5.6 Hz, 2H), 4.47 (s, 2H), 7.10 (t, J=8 Hz, 3H).

LCMS (Method A): 1.165 min, MS: ES+ 346 (M+1)

### Step 4: 7-((4-Methyl piperazin-1-yl)methyl)-1,2,3,4-tetrahydroisoquinoline hydrochloride

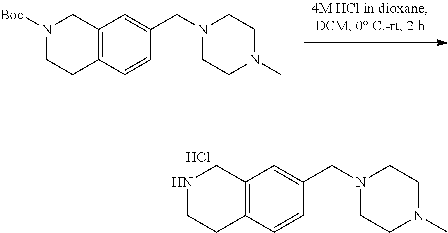 [SMILES: CN1CCN(Cc2ccc3c(c2)CN(C(=O)OC(C)(C)C)CC3)CC1 ; Cl ; CN1CCN(Cc2ccc3c(c2)CNCC3)CC1]

The title compound was prepared from tert-butyl 7-((4-methylpiperazin-1-yl)methyl)-3,4-dihydroisoquinoline-2(1H)-carboxylate (step 3) and 4M HCl in dioxane analogously to Intermediate R step 5.

¹H NMR (DMSO-d6, 400 MHz): δ 1.37-1.43 (m, 2H), 2.79 (s, 3H), 3.03 (t, J=5.6 Hz, 1H), 3.34-3.73 (m, 9H), 4.24-4.50 (m, 4H), 7.23-7.56 (m, 3H), 9.73 (s, 1H), 11.98-12.05 (m, 1H). HCl salt

LCMS (Method B): 1.43 min, MS: ES+ 246.4 (M+1).

### Intermediate YG: 6-((4-Methyl piperazin-1-yl)methyl)-1,2,3,4-tetrahydroisoquinoline hydrochloride

### Step 1: tert-Butyl 6-(hydroxymethyl)-3,4-dihydroisoquinoline-2(1H)-carboxylate

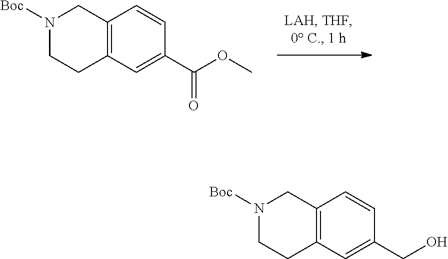 [SMILES: COC(=O)c1ccc2c(c1)CCN(C(=O)OC(C)(C)C)C2 ; CC(C)(C)OC(=O)N1CCc2cc(CO)ccc2C1]

The title compound was prepared from 2-(tert-butyl) 6-methyl 3,4-dihydroisoquinoline-2,6(1H)-dicarboxylate (Intermediate YC step 1) and LAH solution (1M in THF) analogously to Intermediate YF step 1.

¹H NMR (DMSO-d6, 400 MHz): δ 1.42 (s, 9H), 2.74-2.79 (m, 2H), 3.53 (t, J=5.6 Hz, 2H), 4.41-4.46 (m, 4H), 5.13 (t, J=5.6 Hz, 1H), 6.98-7.03 (m, 1H), 7.10 (d, J=3.6 Hz, 2H).

LCMS (Method A): 1.678 min, MS ES+: 163.8 (M−100).

### Step 2: tert-Butyl 6-(bromomethyl)-3,4-dihydroisoquinoline-2(1H)-carboxylate

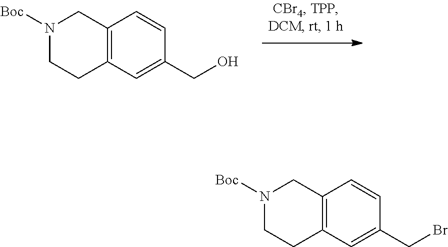 [SMILES: CC(C)(C)OC(=O)N1CCc2cc(CO)ccc2C1 ; CC(C)(C)OC(=O)N1CCc2cc(CBr)ccc2C1]

The title compound was prepared from tert-butyl 6-(hydroxymethyl)-3,4-dihydroisoquinoline-2(1H)-carboxylate (step 1), CBr₄ and triphenyl phosphine analogously to Intermediate R step 3.

¹H NMR (DMSO-d6, 400 MHz): δ 1.42 (s, 9H), 2.74-2.80 (m, 2H), 3.53-3.57 (m, 2H), 4.48-4.52 (m, 2H), 4.67 (s, 2H), 7.16 (d, J=8 Hz, 1H), 7.24-7.29 (m, 2H).

### Step 3: tert-Butyl 6-((4-methylpiperazin-1-yl)methyl)-3,4-dihydroisoquinoline-2(1H)-carboxylate

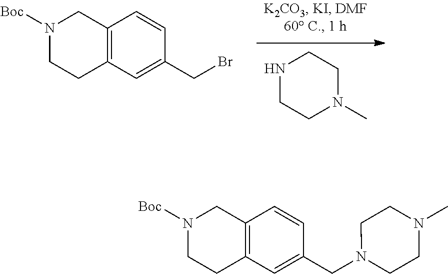

The title compound was prepared from tert-butyl 6-(bromomethyl)-3,4-dihydroisoquinoline-2(1H)-carboxylate (step 2) and 1-methylpiperazine analogously to Intermediate YF step 3

¹H NMR (DMSO-d6, 400 MHz): δ 1.42 (s, 9H), 2.13 (s, 3H), 2.33 (s, 6H), 2.73-2.76 (m, 2H), 3.38 (s, 2H), 3.53 (t, J=5.6 Hz, 2H), 4.46 (s, 2H), 7.06-7.09 (m, 3H),

LCMS (Method A): 1.143 min, MS ES+: 346.0 (M+1).

### Step 4: 6-((4-Methyl piperazin-1-yl)methyl)-1,2,3,4-tetrahydroisoquinoline hydrochloride

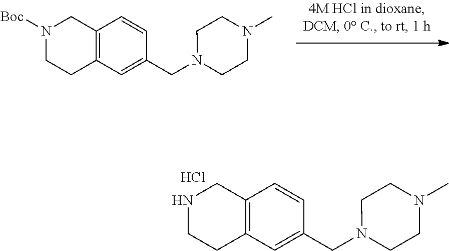 [SMILES: CN1CCN(Cc2ccc3c(c2)CCN(C(=O)OC(C)(C)C)C3)CC1 ; Cl ; CN1CCN(Cc2ccc3c(c2)CCNC3)CC1]

The title compound was prepared from tert-butyl 6-((4-methylpiperazin-1-yl)methyl)-3,4-dihydroisoquinoline-2(1H)-carboxylate (step 3) and 4M HCl in dioxane analogously to Intermediate R step 5.

¹H NMR (DMSO-d6, 400 MHz): δ 2.67-2.85 (bs, 2H), 3.01 (t, J=6 Hz, 2H), 3.35 (bs, 3H), 4.27 (s, 2H), 7.27-7.43 (m, 3H), 9.50 (s, 1H).

LCMS (Method A): 1.59 min, MS ES+: 246.4 (M+1).

### Intermediate YH: 7-((1H-Imidazol-1-yl)methyl)-1,2,3,4-tetrahydroisoquinoline hydrochloride

### Step 1: tert-Butyl 7-((1H-imidazol-1-yl)methyl)-3,4-dihydroisoquinoline-2(1H)-carboxylate

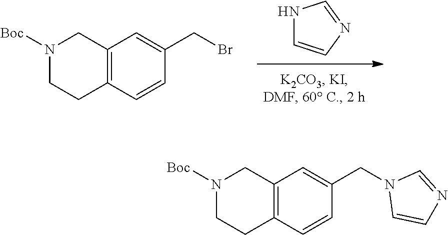 [SMILES: CC(C)(C)OC(=O)N1CCc2ccc(CBr)cc2C1 ; c1c[nH]cn1 ; CC(C)(C)OC(=O)N1CCc2ccc(Cn3ccnc3)cc2C1]

The title compound was prepared from tert-butyl 7-(bromomethyl)-3,4-dihydroisoquinoline-2(1H)-carboxylate (Intermediate YF step 2) and 1H-imidazole analogously to Intermediate YF step 3.

¹H NMR (DMSO-d6, 400 MHz): δ 1.42 (s, 9H), 2.50 (t, J=2 Hz, 2H), 3.52 (t, J=6 Hz, 2H), 4.46 (s, 2H), 5.12 (s, 2H), 6.89 (s, 1H), 7.02-7.17 (m, 4H), 7.74 (s, 1H),

LCMS (Method A): 1.277 min, MS: ES+ 314 (M+1).

### Step 2: 7-((1H-Imidazol-1-yl)methyl)-1,2,3,4-tetrahydroisoquinoline hydrochloride

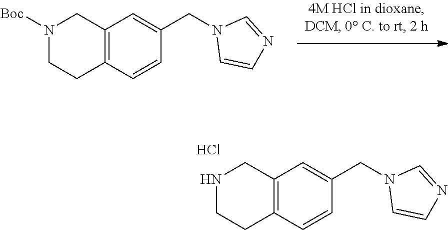 [SMILES: CC(C)(C)OC(=O)N1CCc2ccc(Cn3ccnc3)cc2C1 ; Cl ; c1cn(Cc2ccc3c(c2)CNCC3)cn1]

The title compound was prepared from tert-butyl 7-((1H-imidazol-1-yl)methyl)-3,4-dihydroisoquinoline-2(1H)-carboxylate (step 1) and 4M HCl in dioxane analogously to Intermediate R step 5.

¹H NMR (DMSO-d6, 400 MHz): δ 3.00 (t, J=6 Hz, 2H), 3.30 (t, J=12.4 Hz, 2H), 4.20 (d, J=4.4 Hz, 2H), 5.42 (s, 2H), 7.18-7.34 (m, 3H), 7.70 (t, J=1.6 Hz, 1H), 7.80 (t, J=1.6 Hz, 1H), 9.38 (s, 1H), 9.86 (s, 1H).

LCMS (Method A): 0.180 min, MS: ES+ 213.9 (M+1).

### Intermediate YI: 6-((1H-Imidazol-1-yl)methyl)-1,2,3,4-tetrahydroisoquinoline hydrochloride

### Step 1: tert-Butyl 6-((1H-imidazol-1-yl)methyl)-3,4-dihydroisoquinoline-2(1H)-carboxylate

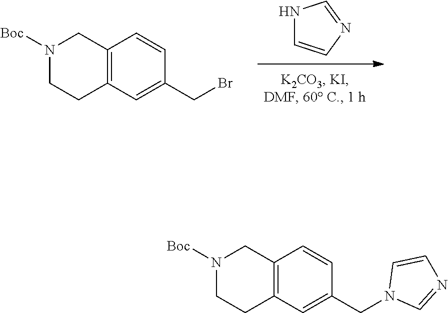 [SMILES: CC(C)(C)OC(=O)N1CCc2cc(CBr)ccc2C1 ; c1c[nH]cn1 ; CC(C)(C)OC(=O)N1CCc2cc(Cn3ccnc3)ccc2C1]

The title compound was prepared from tert-butyl 6-(bromomethyl)-3,4-dihydroisoquinoline-2(1H)-carboxylate (Intermediate YG step 2) and 1H-imidazole analogously to Intermediate YF step 3.

¹H NMR (DMSO-d6, 400 MHz): δ 1.41 (s, 9H), 3.73 (t, J=6 Hz, 2H), 3.52 (t, J=5.6 Hz, 2H), 4.46 (s, 2H), 5.13 (s, 2H), 6.88 (s, 1H), 7.07 (d, J=9.6 Hz, 2H), 7.15 (d, J=8 Hz, 2H), 7.73 (s, 1H), 11.55 (s, 1H), 11.92 (s, 1H), 13.77 (s, 1H).

LCMS (Method A): 1.256 min, MS: ES+ 314.0 (M+1).

### Step 2: 6-((1H-Imidazol-1-yl)methyl)-1,2,3,4-tetrahydroisoquinoline hydrochloride

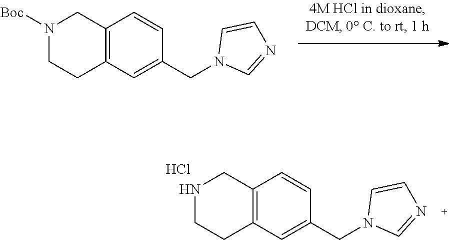 [SMILES: CC(C)(C)OC(=O)N1CCc2cc(Cn3ccnc3)ccc2C1 ; Cl ; c1cn(Cc2ccc3c(c2)CCNC3)cn1]

The title compound was prepared from tert-butyl 6-((1H-imidazol-1-yl)methyl)-3,4-dihydroisoquinoline-2(1H)-carboxylate (step 1) and 4M HCl in dioxane analogously to Intermediate R step 5.

¹H NMR (DMSO-d6, 400 MHz): δ 2.99 (t, J=6.4 Hz, 2H), 3.33 (d, J=4 Hz, 2H), 4.24 (t, J=4.4 Hz, 2H), 5.41 (s, 2H), 7.25-7.31 (m, 3H), 7.69-7.70 (m, 1H), 7.79 (s, 1H), 9.33 (s, 1H), 9.56 (bs, 2H).

LCMS (Method A): 1.53 min, MS: ES+ 214.3 (M+1).

### Intermediate YJ: 5-((1H-Imidazol-1-yl)methyl)isoindoline hydrochloride

### Step 1: tert-Butyl 5-((1H-imidazol-1-yl)methyl)isoindoline-2-carboxylate

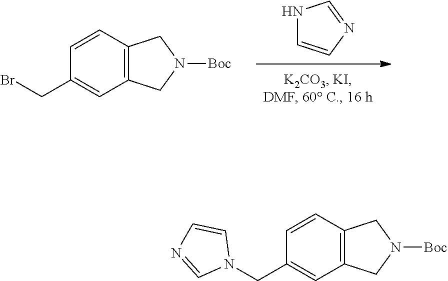 [SMILES: CC(C)(C)OC(=O)N1Cc2ccc(CBr)cc2C1 ; c1c[nH]cn1 ; CC(C)(C)OC(=O)N1Cc2ccc(Cn3ccnc3)cc2C1]

The title compound was prepared from tert-butyl 5-(bromomethyl)isoindoline-2 carboxylate (Intermediate R step 3) and 1H-imidazole analogously to Intermediate YF step 3.

¹H NMR (DMSO-d6, 400 MHz): δ 1.45 (s, 9H), 4.55 (d, J=8.8 Hz, 4H), 5.18 (s, 2H), 6.89 (s, 1H), 7.17-7.24 (m, 3H), 7.29-7.31 (m, 1H), 7.74 (s, 1H).

LCMS (Method A): 1.212 min, MS ES+: 299.9 (M+1).

### Step 2: 5-((1H-Imidazol-1-yl)methyl)isoindoline hydrochloride

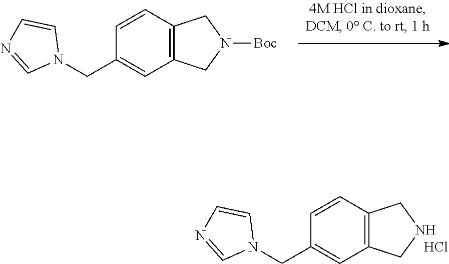 [SMILES: CC(C)(C)OC(=O)N1Cc2ccc(Cn3ccnc3)cc2C1 ; c1cn(Cc2ccc3c(c2)CNC3)cn1 ; Cl]

The title compound was prepared from tert-butyl 5-((1H-imidazol-1-yl)methyl)isoindoline-2-carboxylate (step 1) and 4M HCl in dioxane analogously to Intermediate R step 5.

¹H NMR (DMSO-d6, 400 MHz): δ 4.48 (t, J=5.2 Hz, 10.8 Hz, 4H), 5.48 (s, 2H), 7.42-4.47 (m, 3H), 7.71-7.80 (m, 2H), 9.34 (s, 1H), 10.11 (s, 2H).

LCMS (Method A): 0.180 min, MS ES+: 199.9 (M+1).

### Intermediate YK: 1-(6,7-Dihydro-5H-pyrrolo[3,4-b]pyridin-3-yl)-N,N-dimethylmethanamine hydrochloride

### Step 1a: Dimethyl 5-bromopyridine-2,3-dicarboxylate

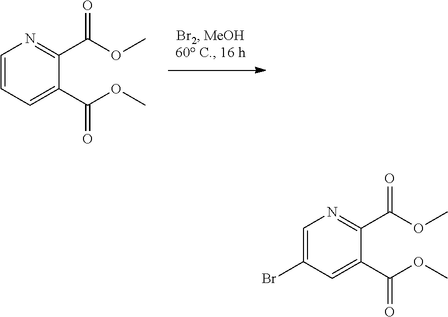

To a stirred solution of dimethyl pyridine-2,3-dicarboxylate (CAS: 605-38-9) (50 g, 256 mmol, 1 eq) in MeOH (500 mL) was added dropwise Br₂ (172 g, 1075 mmol, 4.2 eq) at 0° C. The reaction mixture was allowed to stir at 60° C. for 16 h. The resulting mixture poured into saturated ice-cold solution of thiosulphate (1000 mL) and extracted with EtOAc (3×300 mL). The combined organic extracts were dried over Na₂SO₄, filtered and concentrated under vacuum. The crude material was purified by chromatography on silica, eluting with 25% EtOAc in hexane yielding dimethyl 5-bromopyridine-2,3-dicarboxylate (45 g, Yield: 64.0%).

¹H NMR (DMSO-d6, 400 MHz): δ 3.87 (d, J=2.8 Hz, 6H), 8.53 (d, J=2.0 Hz, 1H), 8.99 (d, J=2.0 Hz, 1H).

LCMS (Method A): 1.480 min, MS: ES+ 273.7 & 275.7 (M+1 & M+3)

### Step 1b: (5-Bromopyridine-2,3-diyl)dimethanol

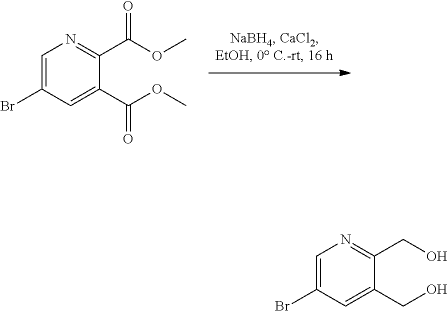

To a cooled (0° C.) solution of dimethyl 5-bromopyridine-2,3-dicarboxylate (step 1a) (30 g, 109.8 mmol, 1 eq) in EtOH (750 mL) was added dropwise NaBH₄ (24.4 g, 659.3 mmol, 6 eq) followed by a solution of CaCl₂ (36.56 g, 329.4 mmol, 3 eq) in EtOH. The reaction mixture was allowed to warm to room temperature and stirred for 16 h. The resulting mixture filtered and the filtrate was concentrated under vacuum. The residue was diluted with water (500 mL) and extracted with EtOAc (7×200 mL). The combined organic extracts were dried over Na₂SO₄, filtered and concentrated under vacuum yielding (5-bromopyridine-2,3-diyl)dimethanol. (22 g, Yield: 46.0%).

LCMS (Method A): 0.691 min, 0.806 min.

### Step 1c: 5-Bromo-2,3-bis(chloromethyl)pyridine

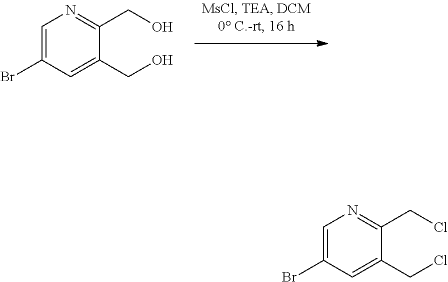 [SMILES: OCc1cc(Br)cnc1CO ; ClCc1cc(Br)cnc1CCl]

To a stirred solution of (5-bromopyridine-2,3-diyl)methanol (step 1 b) (22 g, 101.38 mmol, 1 eq.) in DCM (330 mL) was added TEA (25.6 g, 253.4 mmol, 2.5 eq) and the mixture was stirred at room temperature for 15 mins. Mesyl chloride (34.98 g, 304.14 mmol, 3.0 eq) was added dropwise to the reaction mixture at 0° C. The reaction mixture was stirred at room temperature for 16 h. The resulting mixture was diluted with water (400 mL) and extracted with DCM (2×100 mL). The combined organic extracts were dried over Na₂SO₄, filtered, and concentrated under vacuum. The crude material was purified by chromatography on silica, eluting with 2% EtOAc in hexane yielding to 5-bromo-2,3-bis(chloromethyl)pyridine (10 g, Yield: 39.00%).

¹H NMR (DMSO-d6, 400 MHz): δ 4.89 (s, 2H), 4.93 (s, 2H), 8.25 (d, J=2.4 Hz, 1H), 8.72 (d, J=2.4 Hz, 1H).

LCMS (Method A): 1.867 min, MS: ES+ 253.7 & 253.7 (M+1 & M+3)

### Step 1 d: 3-Bromo-6-(4-methoxybenzyl)-6,7-dihydro-5H-pyrrolo[3,4-b]pyridine

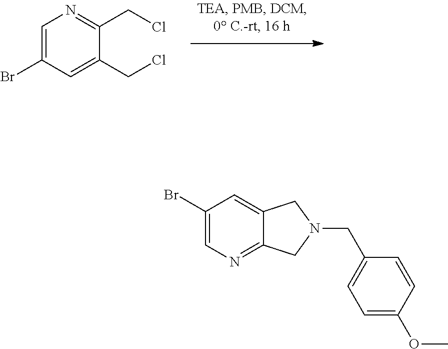 [SMILES: ClCc1cc(Br)cnc1CCl ; COc1ccc(CN2Cc3cc(Br)cnc3C2)cc1]

To a stirred solution of 5-bromo-2,3-bis(chloromethyl)pyridine (step 1c) (6 g, 23.7 mmol, 1 eq.) in DCM (90 mL) was added TEA (7.18 g, 71.1 mmol, 3 eq) and stirring continued at room temperature for 30 min. PMB-Cl (8.12 g, 59.2 mmol, 2.5 eq) was added dropwise to the reaction mixture at 0° C. and the mixture was allowed to stir at room temperature for 16 h. The resulting mixture was diluted with water (100 mL) and extracted with DCM (2×50 mL). The combined organic extracts were dried over Na₂SO₄, filtered and concentrated under vacuum. The crude material was purified by chromatography on silica, eluting with 9% EtOAc in hexane yielding 3-bromo-6-(4-methoxybenzyl)-6,7-dihydro-5H-pyrrolo[3,4-b]pyridine (3.6 g, Yield: 49.0%).

¹H NMR (DMSO-d6, 400 MHz): δ 3.75 (s, 3H), 3.80 (s, 4H), 3.83 (s, 2H), 6.91 (d, J=8.4 Hz, 2H), 7.29 (d, J=8.4 Hz, 2H), 7.92 (d, J=1.6 Hz, 1H), 8.46 (d, J=2.0 Hz, 1H).

LCMS (Method A): 1.107 min, MS: ES+ 318.8 & 320.7 (M+1 & M+3)

### Step 1e: 4-Bromo-6-methoxyisoindoline hydrochloride

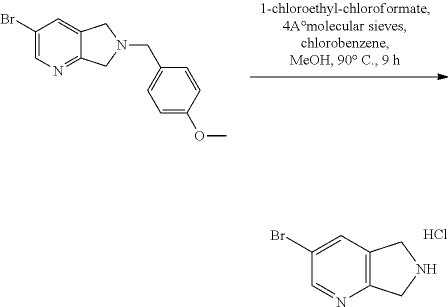 [SMILES: COc1ccc(CN2Cc3cc(Br)cnc3C2)cc1 ; Cl ; Brc1cnc2c(c1)CNC2]

To a stirred solution of 3-bromo-6-(4-methoxybenzyl)-6,7-dihydro-5H-pyrrolo[3,4-b]pyridine (step 1d) (3.5 g, 11 mmol, 1 eq) in chlorobenzene (35 mL) was added 4A° molecule sieves (1.75 g, 50% w\w) and the mixture was stirred at room temperature for 45 min. 1-Chloroethyl chloroformate (3.15 g, 22 mmol, 2 eq) was added dropwise and the reaction mixture was heated to 90° C. for 6 h. After cooling to room temperature, the mixture was filtered and the filter bed washed with MeOH (35 mL). The filtrate was heated to 70° C. for 2 h. The resulting mixture was diluted with water (70 mL) and washed with DCM (3×50 mL). The aqueous portion was concentrated under vacuum yielding 4-bromo-6-methoxyisoindoline hydrochloride as a brown solid (3 g, Yield: Quantitative, 12.82 mmol)

¹H NMR (DMSO-d6, 400 MHz): δ 4.44 (t, J=5.6 Hz, 2H), 4.57 (t, J=5.2 Hz, 2H), 8.14 (d, J=1.2 Hz, 1H), 8.66 (d, J=1.2 Hz, 1H), 10.29 (bs, 2H).

LCMS (Method A): 0.456 min, MS: ES+ 199 & 201 (M+1 & M+3).

### Step 1f: tert-Butyl 3-bromo-5,7-dihydro-6H-pyrrolo[3,4-b]pyridine-6-carboxylate

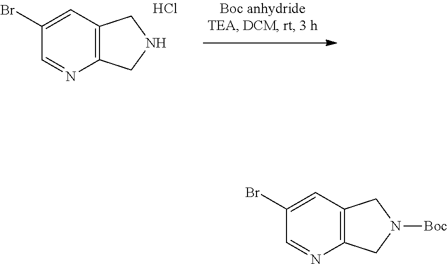 [SMILES: Cl ; Brc1cnc2c(c1)CNC2 ; CC(C)(C)OC(=O)N1Cc2cc(Br)cnc2C1]

To a stirred solution of 4-bromo-6-methoxyisoindoline hydrochloride (step 1e) (6 g, 25.6 mmol, 1 eq) in DCM (120 mL) was added TEA (7.76 g, 76.8 mmol, 3 eq) at room temperature and the mixture was stirred for 15 min. Boc-anhydride (8.38 g, 38.4 mmol, 1.5 eq) was added to the reaction mixture at 0° C. The reaction mixture was allowed to stir at room temperature for 2 h. The resulting mixture was diluted with water (100 mL) and extracted with DCM (5×70 mL). The combined organic extracts were dried over Na₂SO₄, filtered and concentrated under vacuum. The crude material was purified by chromatography on silica, eluting with 12% EtOAc in hexane yielding tert-butyl 3-bromo-5,7-dihydro-6H-pyrrolo[3,4-b]pyridine-6-carboxylate (3.6 g, Yield: 78.0%).

¹H NMR (DMSO-d6, 400 MHz): δ 1.47 (s, 9H), 4.52 (d, J=10.4 Hz, 2H), 4.61 (d, J=12 Hz, 2H), 8.06 (d, J=8 Hz, 1H), 8.58 (d, J=1.6 Hz, 1H).

LCMS (Method A): 1.909 min, MS: ES+ 298.8 & 300.7 (M+1 & M+3).

### Step 1: 6-(tert-Butyl) 3-ethyl 5,7-dihydro-6H-pyrrolo[3,4-b]pyridine-3,6-dicarboxylate

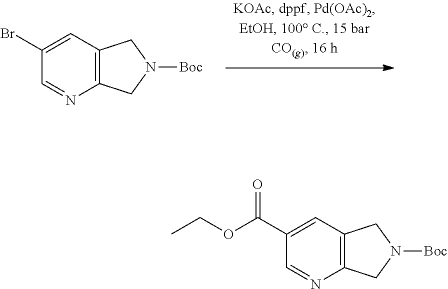 [SMILES: CC(C)(C)OC(=O)N1Cc2cc(Br)cnc2C1 ; CCOC(=O)c1cnc2c(c1)CN(C(=O)OC(C)(C)C)C2]

The title compound was prepared from tert-butyl 3-bromo-5,7-dihydro-6H-pyrrolo[3,4-b]pyridine-6-carboxylate (step 1f), DPPF and Pd(OAc)₂ analogously to Intermediate T step 2.

1H NMR (DMSO-d6, 400 MHz): δ 1.34 (t, J=7.2 Hz, 3H), 1.47 (s, 9H), 4.33-4.38 (m, 2H), 4.62-4.69 (m, 4H), 6.27 (d, J=7.6 Hz, 1H), 8.98 (d, J=1.6 Hz, 1H).

LCMS (Method A): 1.858 min, MS: ES+ 292.9 (M+1)

### Step 2: 3-(Hydroxymethyl)-5,7-dihydro-6H-pyrrolo[3,4-b]pyridine-6-carboxylate

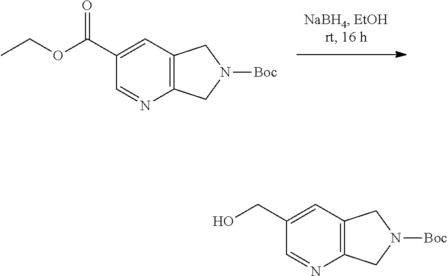 [SMILES: CCOC(=O)c1cnc2c(c1)CN(C(=O)OC(C)(C)C)C2 ; CC(C)(C)OC(=O)N1Cc2cc(CO)cnc2C1]

The title compound was prepared from 6-(tert-butyl) 3-ethyl 5,7-dihydro-6H-pyrrolo[3,4-b]pyridine-3,6-dicarboxylate 9 step 1) and NaBH₄ analogously to Intermediate T step 3.

¹H NMR (DMSO-d6, 400 MHz): δ 1.46 (s, 9H), 4.53 (d, J=6.8 Hz, 4H), 4.61 (d, J=11.2 Hz, 2H), 5.35 (t, J=5.6 Hz, 1H), 7.69 (d, J=8.4 Hz, 1H).

LCMS (Method A): 1.204 min, MS: ES+ 250.9 (M+1).

### Step 3: tert-Butyl 3-formyl-5,7-dihydro-6H-pyrrolo[3,4-b]pyridine-6-carboxylate

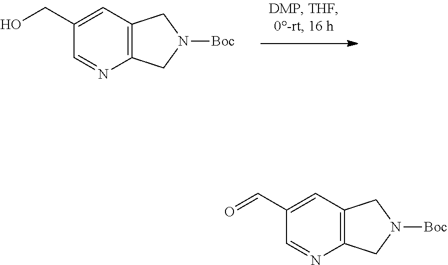 [SMILES: CC(C)(C)OC(=O)N1Cc2cc(CO)cnc2C1 ; CC(C)(C)OC(=O)N1Cc2cc(C=O)cnc2C1]

To a stirred solution of tert-butyl 3-(hydroxymethyl)-5,7-dihydro-6H-pyrrolo[3,4-b]pyridine-6-carboxylate (0.6 g, 2.40 mmol, 1 eq) in THF (20 mL) was added portion wise Dess-Martin periodinane (4.070 g, 9.60 mmol, 4 eq) at 0° C. The reaction mixture was allowed to stir at room temperature for 16 h. The resulting mixture was filtered and the filtrate was diluted with water (50 mL) and extracted with EtOAc (3×30 mL). The combined organic extracts were dried over Na₂SO₄, filtered and concentrated under vacuum. The crude material was purified by chromatography on silica, eluting with 25% EtOAc in hexane yielding tert-butyl 3-formyl-5,7-dihydro-6H-pyrrolo[3,4-b]pyridine-6-carboxylate as an off white solid (0.6 g, Yield: Quantitative, 2.41 mmol)

¹H NMR (DMSO-d6, 400 MHz): δ 1.47 (s, 9H), 4.64-4.71 (m, 4H), 8.20 (d, J=8.0 Hz, 1H), 8.99 (s, 1H), 10.10 (s, 1H).

LCMS (Method A): 1.522 min, MS: ES+ 248.9 (M+1)

### Step 4: tert-Butyl 3-((dimethylamino)methyl)-5,7-dihydro-6H-pyrrolo[3,4-b]pyridine-6-carboxylate

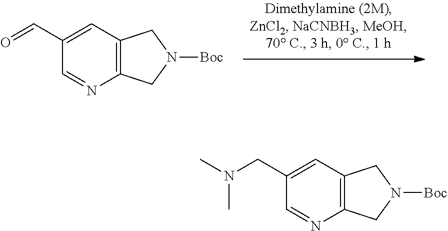 [SMILES: CC(C)(C)OC(=O)N1Cc2cc(C=O)cnc2C1 ; CN(C)Cc1cnc2c(c1)CN(C(=O)OC(C)(C)C)C2]

The title compound was prepared from tert-butyl 3-((dimethylamino)methyl)-5,7-dihydro-6H-pyrrolo[3,4-b]pyridine-6-carboxylate (step 3) and dimethyl amine solution (2M in THF) analogously to Intermediate RA step 2.

¹H NMR (DMSO-d6, 400 MHz): δ 1.46 (s, 9H), 2.14 (s, 6H), 3.42 (s, 2H), 4.52-4.62 (m, 4H), 7.67 (d, J=10 Hz, 1H), 8.34 (s, 1H).

LCMS (Method N): 1.362 min, MS: ES+ 278.3 (M+1).

### Step 5: 1-(6,7-Dihydro-5H-pyrrolo[3,4-b]pyridin-3-yl)-N,N-dimethylmethanamine hydrochloride

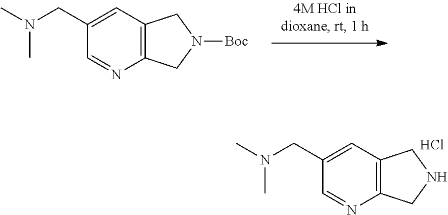 [SMILES: CN(C)Cc1cnc2c(c1)CN(C(=O)OC(C)(C)C)C2 ; CN(C)Cc1cnc2c(c1)CNC2 ; Cl]

The title compound was prepared from tert-butyl 3-((dimethylamino)methyl)-5,7-dihydro-6H-pyrrolo[3,4-b]pyridine-6-carboxylate (step 4) and 4M HCl in dioxane analogously to Intermediate RA step 3.

¹H NMR (DMSO-d6, 400 MHz): δ 2.69 (d, J=4.8 Hz, 6H), 4.36 (d, J=5.2 Hz, 2H), 4.51 (t, J=6.0 Hz, 2H), 4.61 (t, J=5.2 Hz, 2H), 8.08 (d, J=1.6 Hz, 1H), 8.70 (d, J=1.6 Hz, 1H), 10.32 (bs, 2H).

LCMS (Method N): 0.900 min, MS: ES+ 178.3 (M+1).

### Intermediate YL: 2-(Cyclopropylmethoxy)-4-(difluoromethyl)-6-hydroxybenzoic acid

### Step 1: 5-(Cyclopropylmethoxy)-7-(difluoromethyl)-2,2-dimethyl-4H-benzo[d][1,3]dioxin-4-one

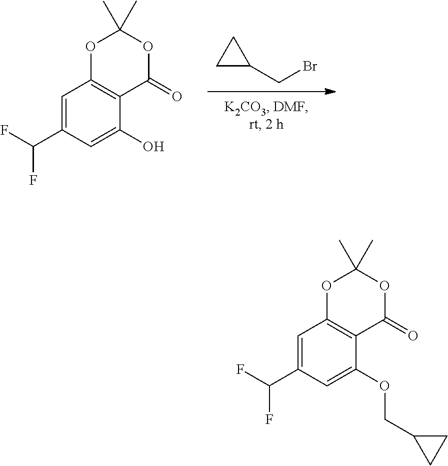 [SMILES: CC1(C)OC(=O)c2c(O)cc(C(F)F)cc2O1 ; BrCC1CC1 ; CC1(C)OC(=O)c2c(OCC3CC3)cc(C(F)F)cc2O1]

The title compound was prepared from 7-(difluoromethyl)-5-hydroxy-2,2-dimethyl-4H-benzo[d][1,3]dioxin-4-one (Intermediate D step 8) and (bromomethyl)cyclopropane (CAS: 7051-34-5) analogously to Intermediate D step 9.

¹H NMR (DMSO-d6, 400 MHz): δ 0.39-0.415 (m, 2H), 0.56-0.60 (m, 2H), 1.24-1.26 (m, 1H), 1.67 (s, 6H), 4.01 (d, J=6.4 Hz, 2H), 6.81 (s, 1H), 6.97 (s, 1H), 7.01 (s, 1H).

LCMS (Method A): 1.80 min, MS: ES+ 220 (M+1).

### Step 2: 2-(Cyclopropylmethoxy)-4-(difluoromethyl)-6-hydroxybenzoic acid

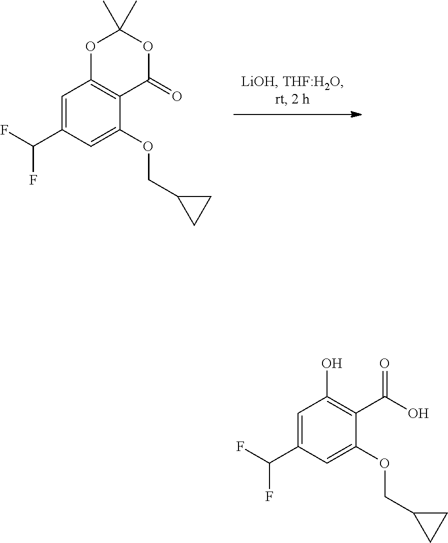 [SMILES: CC1(C)OC(=O)c2c(OCC3CC3)cc(C(F)F)cc2O1 ; O=C(O)c1c(O)cc(C(F)F)cc1OCC1CC1]

The title compound was prepared from 5-(cyclopropylmethoxy)-7-(difluoromethyl)-2,2-dimethyl-4H-benzo[d][1,3]dioxin-4-one (step 1) and LiOH·H₂O analogously to Intermediate D step 10.

¹H NMR (DMSO-d6, 400 MHz): δ 0.39-0.41 (m, 2H), 0.53-0.56 (m, 2H), 1.13-1.23 (m, 1H), 3.86 (d, J=6.8 Hz, 2H), 6.65-7.05 (m, 3H), 10.38 (s, 1H), 12.85 (bs, 1H).

LCMS (Method A): 1.69 min, MS: ES+ 256.8 (M−1).

### Intermediate YM: 5-(Pyridin-4-yloxy)isoindoline hydrochloride

### Step 1: tert-Butyl 5-(pyridin-4-yloxy)isoindoline-2-carboxylate

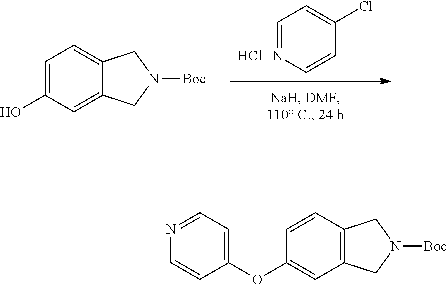 [SMILES: CC(C)(C)OC(=O)N1Cc2ccc(O)cc2C1 ; Cl ; Clc1ccncc1 ; CC(C)(C)OC(=O)N1Cc2ccc(Oc3ccncc3)cc2C1]

To a stirred solution of tert-butyl 5-hydroxyisoindoline-2-carboxylate (Intermediate V step 2) (0.5 g, 2.13 mmol, 1 eq) in DMF (5 mL), cooled to 0° C. was added portion wise NaH (0.17 g, 7.08 mmol, 2 eq) and the mixture was stirred for 30 min. 4-Chloropyridine hydrochloride (0.351 g, 2.34 mmol, 1.1 eq) (CAS: 7379-35-3) was added at 0° C. and the reaction mixture was heated to 110° C. for 24 h. The resulting mixture was diluted using EtOAc (200 mL) and washed with ice cold water (3×100 mL). The combined organic extracts were dried over Na₂SO₄, filtered and concentrated under vacuum. The crude material was purified by chromatography on silica (eluting with 40% EtOAc in hexane) yielding tert-butyl 5-(pyridin-4-yloxy)isoindoline-2-carboxylate as a brown solid (0.1 g, Yield: 15%).

¹H NMR (DMSO-d6, 400 MHz): δ 1.39-1.40 (m, 9H), 4.61 (d, J=9.6 Hz, 4H), 6.93 (d, J=6.0 Hz, 2H), 7.11 (d, J=8.0 Hz, 1H), 7.20 (s, 1H), 7.43-7.46 (m, 1H), 8.47-8.48 (m, 2H).

LCMS (Method A): 1.318 min, MS ES+: 313.0 (M+1).

### Step 2: 5-(Pyridin-4-yloxy)isoindoline hydrochloride

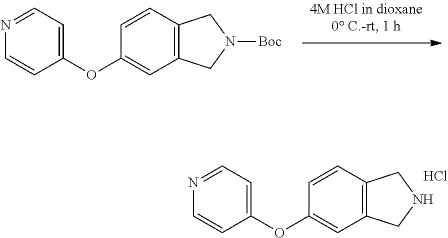 [SMILES: CC(C)(C)OC(=O)N1Cc2ccc(Oc3ccncc3)cc2C1 ; c1cc(Oc2ccc3c(c2)CNC3)ccn1 ; Cl]

To a stirred solution of tert-butyl 5-(pyridin-4-yloxy)isoindoline-2-carboxylate (step 1) (0.1 g, 0.32 mmol, 1 eq) in DCM (1 mL) cooled to 0° C. was added dropwise 4M HCl in dioxane (1.0 mL). The reaction mixture was stirred at room temperature for 1 h. The resulting mixture was concentrated under vacuum. The crude material was triturated with diethyl ether (10 mL) and n-pentane (10 mL) followed by high vacuum drying to yield 5-(pyridin-4-yloxy)isoindoline hydrochloride as a brown solid (0.1 g, Yield: quantitative).

¹H NMR (DMSO-d6, 400 MHz): δ 4.36-4.66 (m, 4H), 6.76-6.79 (m, 1H), 7.32-7.44 (m, 3H), 7.60 (d, J=8.4 Hz, 1H), 8.78 (d, J=6.8 Hz, 2H), 10.00 (s, 1H).

### Intermediate YN: 5-(Pyrimidin-4-yloxy)isoindoline hydrochloride

### Step 1: tert-Butyl 5-(pyrimidin-4-yloxy)isoindoline-2-carboxylate

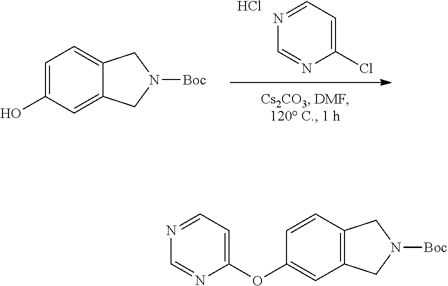 [SMILES: CC(C)(C)OC(=O)N1Cc2ccc(O)cc2C1 ; Cl ; Clc1ccncn1 ; CC(C)(C)OC(=O)N1Cc2ccc(Oc3ccncn3)cc2C1]

To a stirred solution of tert-butyl 5-hydroxyisoindoline-2-carboxylate (Intermediate V step 2) (0.3 g, 1.275 mmol, 1.0 eq.) in DMF (3 mL) were added 4-chloropyrimidine hydrochloride (0.21 g, 1.40 mmol, 1.1 eq.) (CAS: 179051-78-6) and Cs₂CO₃ (1.24 g, 3.82 mmol, 3.0 eq.) at room temperature. The reaction mixture was heated to 120° C. and stirred for 1 h. The resulting mixture was diluted with water (25 mL) and extracted with ethyl acetate (2×30 mL). The combined organic extracts was dried over Na₂SO₄, filtered and concentrated under vacuum yielding tert-butyl 5-(pyrimidin-4-yloxy)isoindoline-2-carboxylate as a brown gum (0.4 g, Yield: 100%).

¹H NMR (DMSO-d6, 400 MHz): δ 1.46 (s, 9H), 4.59-4.61 (m, 4H), 7.09-7.16 (m, 2H), 7.21 (s, 1H), 7.39-7.42 (m, 1H), 8.68 (d, J=6 Hz, 1H), 8.75 (s, 1H).

LCMS (Method A): 1.63 min, MS: ES+313.9 (M+1).

### Step 2: 5-(Pyrimidin-4-yloxy)isoindoline HCl

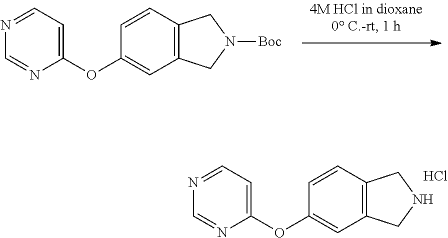 [SMILES: CC(C)(C)OC(=O)N1Cc2ccc(Oc3ccncn3)cc2C1 ; c1cc(Oc2ccc3c(c2)CNC3)ncn1 ; Cl]

The title compound was prepared from tert-butyl 5-(pyrimidin-4-yloxy)isoindoline-2-carboxylate (step 1) and 4M HCl in dioxane analogously to Intermediate YM step 2.

¹H NMR (DMSO-d6, 400 MHz): δ 4.51 (t, J=5.6 Hz, J=11.2 Hz, 4H), 7.21-7.50 (m, 3H), 7.49 (d, J=8 Hz, 1H), 8.74 (d, J=8.7 Hz, 1H), 8.82 (s, 1H), 10.16 (s, 1H).

LCMS (Method B): 1.39 min, MS: ES+214.2 (M+1).

### Intermediate YO: (S)-6-((1-Methylpiperidin-4-yl)oxy)-N-(tetrahydrofuran-3-yl)isoindolin-4-amine hydrochloride

### Step 1: tert-Butyl 4-bromo-6-((1-methylpiperidin-4-yl)oxy)isoindoline-2-carboxylate

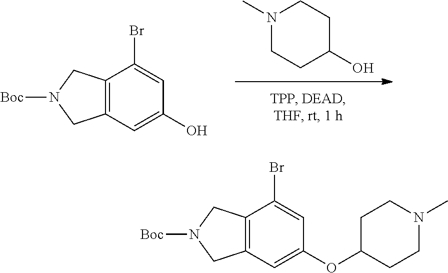

To a stirred solution of tert-butyl 4-bromo-6-hydroxyisoindoline-2-carboxylate (Intermediate H step 2) (1 g, 3.18 mmol, 1 eq) in THF (10 mL) were added 1-methylpiperidin-4-ol (1.09 g, 9.54 mmol, 3 eq) (CAS: 106-52-5), TPP (1.67 g, 6.37 mmol, 2 eq) followed by dropwise addition of DEAD (40% in toluene) (1.28 g, 6.33 mmol, 2 eq) and the reaction mixture was stirred at room temperature for 1 h. The resulting mixture was concentrated under vacuum and the crude material was diluted with water (100 mL) and extracted with EtOAc (3×100 mL). The combined organic extracts were dried over Na₂SO₄, filtered and concentrated under vacuum. The crude material was purified by chromatography on silica (eluting with 10% MeOH in DCM) yielding tert-butyl 4-bromo-6-((1-methylpiperidin-4-yl)oxy)isoindoline-2-carboxylate as yellow oil (0.75 g, Yield: 57%).

¹H NMR (DMSO-d6, 400 MHz): δ 1.46 (s, 9H), 1.59-1.64 (m, 2H), 1.89-1.91 (m, 2H), 2.57-2.58 (m, 2H), 2.17 (s, 5H), 3.17 (d, J=4.8 Hz, 1H), 4.09-4.13 (m, 1H), 4.37-4.45 (m, 3H), 4.63 (d, J=10.4 Hz, 2H), 6.98 (d, J=5.6 Hz, 1H), 7.09 (s, 1H).

LCMS (Method A): 1.435 min, MS ES+: 412.9 (M+2).

### Step 2: tert-Butyl (S)-6-((1-m ethylpiperidin-4-yl)oxy)-4-((tetrahydrofuran-3-yl)amino)isoindoline-2-carboxylate

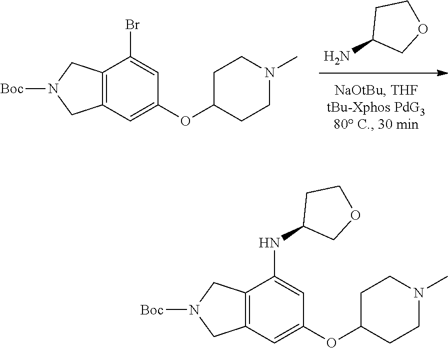 [SMILES: CN1CCC(Oc2cc(Br)c3c(c2)CN(C(=O)OC(C)(C)C)C3)CC1 ; N[C@H]1CCOC1 ; CN1CCC(Oc2cc3c(c(N[C@H]4CCOC4)c2)CN(C(=O)OC(C)(C)C)C3)CC1]

The title compound was prepared from tert-butyl 4-bromo-6-((1-m ethylpiperidin-4-yl)oxy)isoindoline-2-carboxylate (step 1) and (S)-tetrahydrofuran-3-amine analogously to Intermediate B1-1 step 1.

¹H NMR (DMSO-d6, 400 MHz): δ 1.4-1.46 (m, 9H), 1.71-1.72 (m, 2H), 1.82-1.84 (m, 1H), 1.91 (s, 1H), 1.95-1.96 (m, 2H), 2.13-2.18 (m, 1H), 2.43 (s, 3H), 2.88-2.89 (m, 2H), 3.17 (s, 1H), 3.54-3.58 (m, 1H), 3.69-3.74 (m, 2H), 3.78-3.84 (m, 1H), 3.85-3.93 (m, 1H), 3.95-4.05 (m, 1H), 4.32-4.36 (m, 3H), 4.44 (d, J=11.6 Hz, 2H), 5.41-5.45 (m, 1H), 5.99 (s, 1H), 6.20 (d, J=8 Hz, 1H).

LCMS (Method A): 1.249 min, MS ES+: 418.2 (M+1).

### Step 3: (S)-6-((1-Methylpiperidin-4-yl)oxy)-N-(tetrahydrofuran-3-yl)isoindolin-4-amine hydrochloride

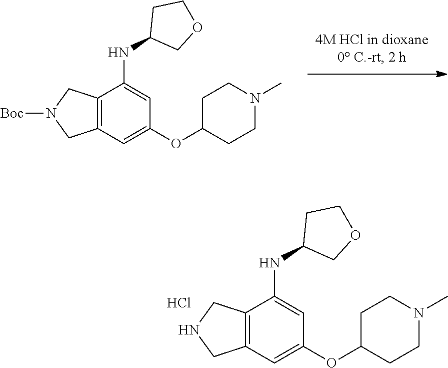 [SMILES: CN1CCC(Oc2cc3c(c(N[C@H]4CCOC4)c2)CN(C(=O)OC(C)(C)C)C3)CC1 ; Cl ; CN1CCC(Oc2cc3c(c(N[C@H]4CCOC4)c2)CNC3)CC1]

The title compound was prepared from tert-butyl (S)-6-((1-methylpiperidin-4-yl)oxy)-4-((tetrahydrofuran-3-yl)amino)isoindoline-2-carboxylate (step 2) and 4M HCl in dioxane analogously to Intermediate YM step 2.

¹H NMR (DMSO-d6, 400 MHz): δ 1.78-1.85 (m, 2H), 2.02-2.03 (m, 2H), 2.16-2.21 (m, 2H), 2.72-2.76 (m, 3H), 3.00-3.10 (m, 2H), 3.20-3.30 (m, 1H), 3.40-3.50 (m, 1H), 3.54-3.57 (m, 5H), 3.70-3.74 (m, 2H), 3.82-3.92 (m, 2H), 4.00-4.08 (m, 1H), 4.20-4.30 (m, 2H), 4.31-4.40 (m, 2H), 6.05-6.16 (m, 1H), 6.31 (d, J=8.8 Hz, 1H).

LCMS (Method A): 1.456 min, MS ES+: 318.35 (M+1)

### Intermediate YP: (R)-6-((1-Methylpiperidin-4-yl)oxy)-N-(tetrahydrofuran-3-yl)isoindolin-4-amine hydrochloride

### Step 1: tert-Butyl (R)-6-((1-methylpiperidin-4-yl)oxy)-4-((tetrahydrofuran-3-yl)amino)isoindoline-2-carboxylate

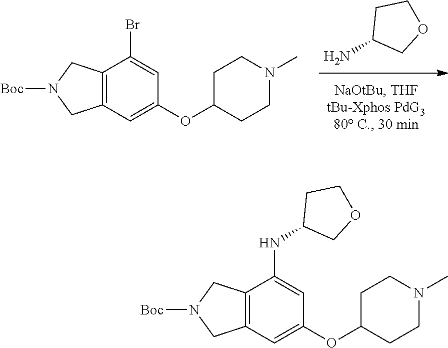 [SMILES: CN1CCC(Oc2cc(Br)c3c(c2)CN(C(=O)OC(C)(C)C)C3)CC1 ; N[C@@H]1CCOC1 ; CN1CCC(Oc2cc3c(c(N[C@@H]4CCOC4)c2)CN(C(=O)OC(C)(C)C)C3)CC1]

The title compound was prepared from tert-butyl 4-bromo-6-((1-m ethylpiperidin-4-yl)oxy)isoindoline-2-carboxylate (Intermediate YO step 1) and (R)-tetrahydrofuran-3-amine (CAS: 111769-26-7) analogously to Intermediate B1-1 step 1.

¹H NMR (DMSO-d6, 400 MHz) (D2O Exchange): δ 1.40 (s, 9H), 1.70-1.90 (m, 3H), 1.91 (s, 1H), 1.95-2.10 (m, 2H), 2.12-2.30 (m, 2H), 2.60-2.90 (m, 1H), 2.95-3.10 (m, 2H), 3.30-3.40 (m, 1H), 3.30-3.40 (m, 1H), 3.65-3.70 (m, 2H), 3.70-3.80 (m, 1H), 3.85-3.86 (m, 1H), 3.90-4.01 (m, 1H), 4.30-4.40 (m, 2H), 4.50-4.60 (m, 3H), 5.99 (s, 1H), 6.22 (d, J=10.4 Hz, 1H).

LCMS (Method A): 1.253 min, MS ES+: 418.2 (M+1).

### Step 2: (R)-6-((1-Methylpiperidin-4-yl)oxy)-N-(tetrahydrofuran-3-yl)isoindolin-4-amine hydrochloride

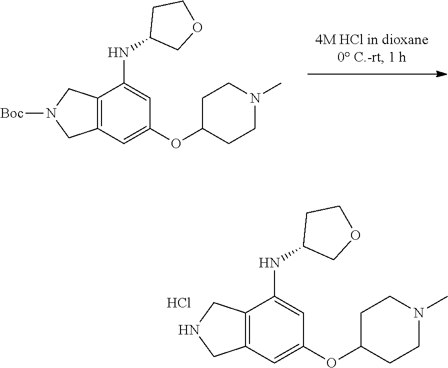 [SMILES: CN1CCC(Oc2cc3c(c(N[C@@H]4CCOC4)c2)CN(C(=O)OC(C)(C)C)C3)CC1 ; Cl ; CN1CCC(Oc2cc3c(c(N[C@@H]4CCOC4)c2)CNC3)CC1]

The title compound was prepared from tert-butyl (R)-6-((1-methylpiperidin-4-yl)oxy)-4-((tetrahydrofuran-3-yl)amino)isoindoline-2-carboxylate (step 1) and 4M HCl in dioxane analogously to Intermediate YM step 2.

¹H NMR (DMSO-d6, 400 MHz) δ1.55 (s, 1H), 1.70-1.90 (m, 3H), 1.93-2.10 (m, 3H), 2.15-2.30 (m, 3H), 2.77 (d, J=9.2 Hz, 4H), 3.00-3.20 (m, 3H), 3.25-3.33 (m, 1H), 3.36-3.41 (m, 1H), 3.45-3.51 (m, 2H), 3.55 (s, 6H), 3.80-3.90 (m, 3H), 4.00-4.05 (m, 1H), 4.10-4.15 (m, 1H), 4.25 (s, 2H), 4.35-4.40 (m, 2H), 4.45-4.60 (m, 3H), 4.60-4.65 (m, 1H), 5.99-6.10 (m, 1H), 6.30-6.34 (m, 1H). 1H NM R looks complex in aliphatic region.

LCMS (Method A): 1.453 min, MS ES+: 318.3 (M+1).

### Intermediate YQ: (S)-6-((1-Methylpyrrolidin-3-yl)oxy)-1,2,3,4-tetrahydroisoquinoline hydrochloride

### Step 1: tert-Butyl (S)-6-((1-methylpyrrolidin-3-yl)oxy)-3,4-dihydroisoquinoline-2(1H)-carboxylate

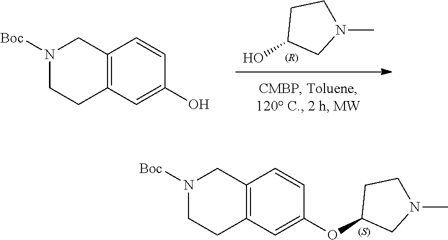

The title compound was prepared from tert-butyl 6-hydroxy-3,4-dihydroisoquinoline-2(1H)-carboxylate (Intermediate VF step 2) and (R)-1-methylpyrrolidin-3-ol analogously to Intermediate YD step 1.

¹H NMR (DMSO-d6, 400 MHz): δ 1.42 (s, 9H), 1.71-1.76 (m, 1H), 2.23-2.29 (m, 4H), 2.33-2.38 (m, 1H), 2.50-2.78 (m, 5H), 3.50 (t, J=5.6 Hz, 2H), 4.40 (s, 2H), 4.80-4.84 (m, 1H), 6.66-6.71 (m, 2H), 7.05 (d, J=8.4 Hz, 1H).

LCMS (Method N): 1.359 min, MS: ES+ 333.1 (M+1)

### Step 2: (S)-6-((1-Methylpyrrolidin-3-yl)oxy)-1,2,3,4-tetrahydroisoquinoline hydrochloride

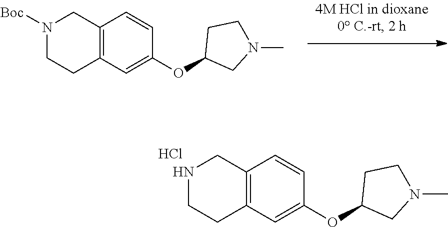 [SMILES: CN1CC[C@H](Oc2ccc3c(c2)CCN(C(=O)OC(C)(C)C)C3)C1 ; Cl ; CN1CC[C@H](Oc2ccc3c(c2)CCNC3)C1]

The title compound was prepared from tert-butyl (S)-6-((1-methylpyrrolidin-3-yl)oxy)-3,4-dihydroisoquinoline-2(1H)-carboxylate (step 1) and 4M HCl in dioxane analogously to Intermediate YD step 2.

LCMS (Method 0): 1.608 min, MS ES+: 233.2 (M+1).

### Intermediate YR: (R)-6-((1-Methylpyrrolidin-3-yl)oxy)-1,2,3,4-tetrahydroisoquinoline hydrochloride

### Step 1: tert-Butyl (R)-6-((1-methylpyrrolidin-3-yl)oxy)-3,4-dihydroisoquinoline-2(1H)-carboxylate

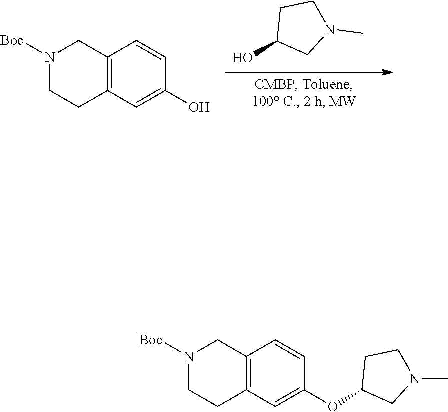

The title compound was prepared from tert-butyl 6-hydroxy-3,4-dihydroisoquinoline-2(1H)-carboxylate (Intermediate VF step 2) and (S)-1-methylpyrrolidin-3-ol analogously to Intermediate YD step 1.

¹H NMR (DMSO-d6, 400 MHz): δ 1.42 (s, 9H), 1.71-1.75 (m, 1H), 2.22-2.28 (m, 4H), 2.33-2.38 (m, 1H), 2.58 (dd J=2.8 Hz, J=2.4 Hz, 1H), 2.61-2.78 (m, 4H), 3.50 (t, J=5.6 Hz, 2H), 4.40 (s, 2H), 4.80-4.83 (m, 1H), 6.62-6.71 (m, 2H), 7.05 (d, J=8.4 Hz, 1H).

LCMS (Method A): 1.276 min, MS: ES+ 333.1 (M+1)

### Step 2: (R)-6-((1-Methylpyrrolidin-3-yl)oxy)-1,2,3,4-tetrahydroisoquinoline hydrochloride

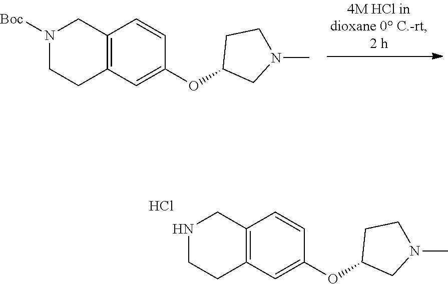 [SMILES: CN1CC[C@@H](Oc2ccc3c(c2)CCN(C(=O)OC(C)(C)C)C3)C1 ; Cl ; CN1CC[C@@H](Oc2ccc3c(c2)CCNC3)C1]

The title compound was prepared from tert-butyl (R)-6-((1-methylpyrrolidin-3-yl)oxy)-3,4-dihydroisoquinoline-2(1H)-carboxylate (step 1) and 4M HCl in dioxane analogously to Intermediate YD step 2.

LCMS (Method O): 1.610 min, MS ES+: 233.2 (M+1).

### Intermediate YS: 6-((1-Methylazetidin-3-yl)oxy)-1,2,3,4-tetrahydroisoquinoline TFA salt

### Step 1: tert-Butyl 6-((1-methylazetidin-3-yl)oxy)-3,4-dihydroisoquinoline-2(1H)-carboxylate

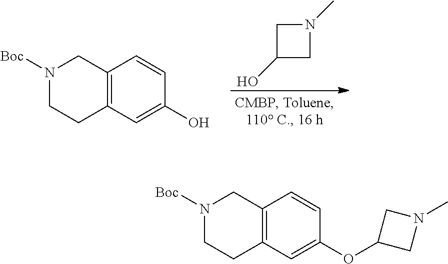

The title compound was prepared from tert-butyl 6-hydroxy-3,4-dihydroisoquinoline-2(1H)-carboxylate (Intermediate VF step 2) and 1-methylazetidin-3-ol analogously to Intermediate YD step 1.

¹H NMR (DMSO-d6, 400 MHz): δ 1.42 (s, 9H), 2.27 (s, 3H), 2.72 (t, J=6.0 Hz, 2H), 2.91-2.94 (m, 2H), 3.50 (t, J=5.6 Hz, 2H), 3.70-3.74 (m, 2H), 4.40 (bs, 2H), 4.68-4.71 (m, 5.6), 6.60-6.66 (m, 2H), 7.06 (d, J=8.4 Hz, 1H).

LCMS (Method A): 1.295 min, MS: ES+ 319.1 (M+1).

### Step 2: 6-((1-Methylazetidin-3-yl)oxy)-1,2,3,4-tetrahydroisoquinoline TFA salt

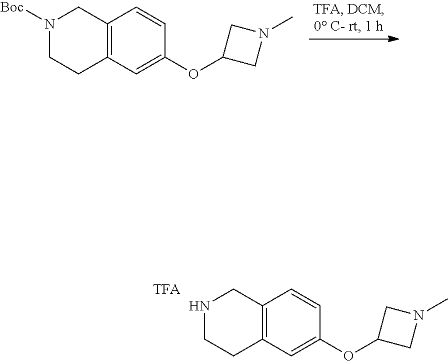 [SMILES: CN1CC(Oc2ccc3c(c2)CCN(C(=O)OC(C)(C)C)C3)C1 ; CN1CC(Oc2ccc3c(c2)CCNC3)C1 ; "TFA"]

The title compound was prepared from tert-butyl (S)-6-((1-methylpyrrolidin-3-yl)oxy)-3,4-dihydroisoquinoline-2(1H)-carboxylate (step 1) and TFA analogously to Intermediate YD step 2.

### Intermediate YT: (R)-7-((1-Methylpyrrolidin-3-yl)oxy)-1,2,3,4-tetrahydroisoquinoline hydrochloride

### Step 1: tert-Butyl (R)-7-((1-methylpyrrolidin-3-yl)oxy)-3,4-dihydroisoquinoline-2(1H)-carboxylate

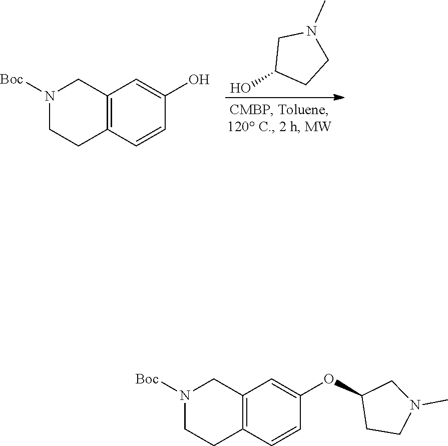

The title compound was prepared from tert-butyl 7-hydroxy-3,4 dihydroisoquinoline-2(1H)-carboxylate (Intermediate YA step 2) and (S)-1-methylpyrrolidin-3-ol analogously to Intermediate YD step 1.

¹H NMR (DMSO-d6, 400 MHz): δ 1.43 (s, 9H), 1.60-1.69 (m, 3H), 1.89-1.91 (m, 2H), 2.22 (bs, 3H), 2.67 (t, J=4.4 Hz, 3H), 3.52 (t, J=5.2 Hz, 2H), 4.43 (bs, 1H), 4.45 (bs, 2H), 6.76 (t, J=8.4 Hz, 2H), 7.04 (d, J=8.4 Hz, 1H). ¹H NMR has trapped impurities of CMBP reagent.

LCMS (Method N): 1.313 min, MS ES+: 333 (M+1).

### Step 2: (R)-7-((1-Methylpyrrolidin-3-yl)oxy)-1,2,3,4-tetrahydroisoquinoline hydrochloride

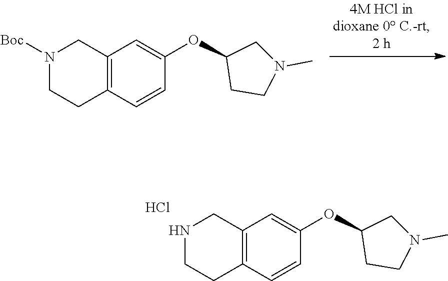 [SMILES: CN1CC[C@@H](Oc2ccc3c(c2)CN(C(=O)OC(C)(C)C)CC3)C1 ; Cl ; CN1CC[C@@H](Oc2ccc3c(c2)CNCC3)C1]

The title compound was prepared from tert-butyl (R)-7-((1-methylpyrrolidin-3-yl)oxy)-3,4-dihydroisoquinoline-2(1H)-carboxylate (step 1) and 4M HCl in dioxane analogously to Intermediate YD step 2.

¹H NMR (DMSO-d6, 400 MHz): δ 1.84-1.90 (m, 1H), 1.93-1.98 (d, J=24 Hz, 1H), 2.02-2.21 (m, 2H), 2.73-2.76 (m, 3H), 2.93 (bs, 2H), 3.01-3.15 (m, 2H), 3.25-3.33 (m, 2H), 3.45 (d, J=11.2 Hz, 1H), 3.65 (bs, 4H), 4.19 (bs, 2H), 4.70 (bs, 1H), 6.90-6.97 (m, 2H), 7.15 (t, J=8.4 Hz, 1H), 10.87 (bs, 1H).

### Intermediate YU: (S)-7-((1-Methylpyrrolidin-3-yl) oxy)-1,2,3,4-tetrahydroisoquinoline hydrochloride

### Step 1: tert-Butyl (S)-7-((1-methylpyrrolidin-3-yl)oxy)-3,4-dihydroisoquinoline-2(1H)-carboxylate

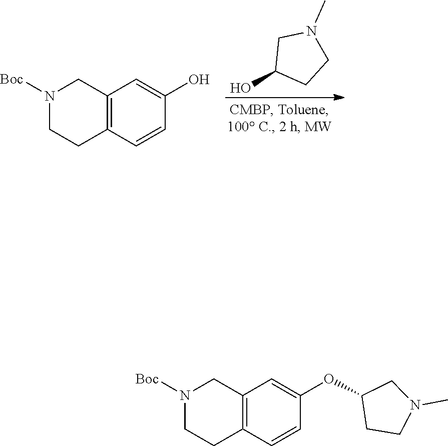

The title compound was prepared from tert-butyl 7-hydroxy-3,4-dihydroisoquinoline-2(1H)-carboxylate (Intermediate YA step 2) and (R)-1-methylpyrrolidin-3-ol analogously to Intermediate YD step 1.

¹H NMR (DMSO-d6, 400 MHz): δ 1.42 (s, 9H), 1.71-1.74 (m, 1H), 2.23-2.38 (m, 5H), 2.54-2.68 (m, 4H), 2.74-2.78 (m, 1H), 3.51 (t, J=5.6 Hz, J=6 Hz, 2H), 4.44 (s, 2H), 4.80-4.83 (m, 1H), 5.76 (d, J=1.6 Hz, 1H), 6.68 (d, J=5.6 Hz, 2H), 7.03 (d, J=8.8 Hz, 1H)

LCMS (Method A): 1.29 min, MS: ES+333.1 (M+1).

### Step 2: (S)-7-((1-Methylpyrrolidin-3-yl) oxy)-1,2,3,4-tetrahydroisoquinoline hydrochloride

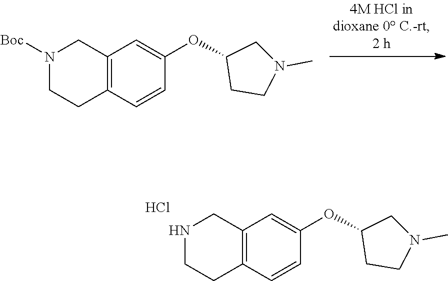 [SMILES: CN1CC[C@H](Oc2ccc3c(c2)CN(C(=O)OC(C)(C)C)CC3)C1 ; Cl ; CN1CC[C@H](Oc2ccc3c(c2)CNCC3)C1]

The title compound was prepared from tert-butyl (S)-7-((1-methylpyrrolidin-3-yl) oxy)-3,4-dihydroisoquinoline-2(1H)-carboxylate (step 1) and 4M HCl in dioxane analogously to Intermediate YD step 2.

LCMS (Method A): 0.37 min, MS: ES+ 232.9 (M+1).

### Intermediate YV: 7-((1-Methylazetidin-3-yl)oxy)-1,2,3,4-tetrahydroisoquinoline hydrochloride

### Step 1: tert-Butyl 7-((1-methylazetidin-3-yl)oxy)-3,4-dihydroisoquinoline-2(1H)-carboxylate

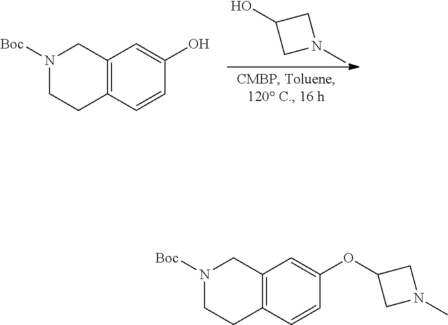

The title compound was prepared from tert-butyl 7-hydroxy-3,4-dihydroisoquinoline-2(1H)-carboxylate (Intermediate YA step 2) and 1-methylazetidin-3-ol analogously to Intermediate YD step 1.

¹H NMR (DMSO-d6, 400 MHz): δ 1.45 (s, 9H), 2.28 (s, 3H), 2.67 (t, J=5.6 Hz, J=11.2 Hz, 2H), 2.91-2.95 (m, 2H), 3.51 (t, J=5.6 Hz, J=11.6 Hz, 2H), 3.73 (t, J=6 Hz, J=8.4 Hz, 2H), 4.44 (s, 2H), 4.68-4.71 (m, 1H), 6.63-6.65 (m, 2H), 7.04 (d, J=8 Hz, 1H).

LCMS (Method A): 1.33 min, MS: ES+ 319.04 (M+1).

### Step 2: 7-((1-Methylazetidin-3-yl)oxy)-1,2,3,4-tetrahydroisoquinoline hydrochloride

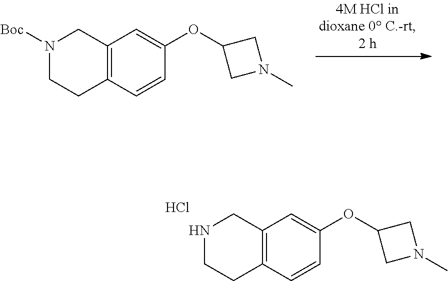 [SMILES: CN1CC(Oc2ccc3c(c2)CN(C(=O)OC(C)(C)C)CC3)C1 ; Cl ; CN1CC(Oc2ccc3c(c2)CNCC3)C1]

The title compound was prepared from tert-butyl 7-((1-methylazetidin-3-yl)oxy)-3,4-dihydroisoquinoline-2(1H)-carboxylate (step 1) and 4M HCl in dioxane analogously to Intermediate YD step 2.

¹H NMR (DMSO-d6, 400 MHz): δ 2.89-2.95 (m, 4H), 3.34 (d, J=5.6 Hz, 2H), 3.58 (s, 2H), 3.98-4.04 (m, 1H), 4.21-4.22 (m, 3H), 4.34-4.39 (m, 1H), 4.69-4.71 (m, 1H), 9.44 (s, 1H).

### Intermediate YW: 4-(2-(Isoindolin-5-yloxy) ethyl) morpholine hydrochloride

### Step 1: tert-Butyl 5-(2-morpholinoethoxy) isoindoline-2-carboxylate

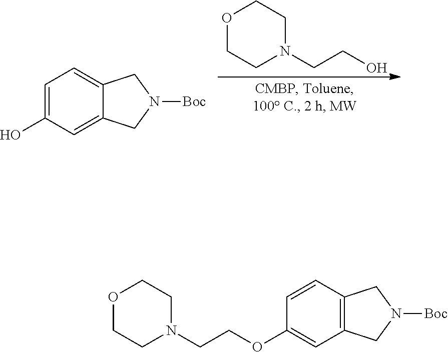 [SMILES: OCCN1CCOCC1 ; CC(C)(C)OC(=O)N1Cc2ccc(O)cc2C1 ; CC(C)(C)OC(=O)N1Cc2ccc(OCCN3CCOCC3)cc2C1]

The title compound was prepared from tert-butyl 5-hydroxyisoindoline-2-carboxylate (Intermediate V step 2) and 2-morpholinoethan-1-ol (CAS; 622-40-2) analogously to Intermediate YD step 1.

¹H NMR (DMSO-d6, 400 MHz): δ 1.31 (s, 9H), 2.45-2.51 (m, 4H), 2.67 (t, J=6.0 Hz, J=11.6 Hz, 2H), 3.57 (t, J=4.4 Hz, J=9.2 Hz, 4H), 4.06 (t, J=4.4 Hz, J=10.0 Hz, 2H), 4.51 (t, J=11.6 Hz, J=25.2 Hz, 4H), 6.85 (d, J=8.0 Hz, 1H), 6.91 (d, J=7.2 Hz, 1H), 7.19-7.21 (dd, J=5.6 Hz, J=8.4 Hz, 1H).

LCMS (Method N): 1.308 min, MS: ES+ 349.20 (M+1).

### Step 2: 4-(2-(Isoindolin-5-yloxy) ethyl) morpholine hydrochloride

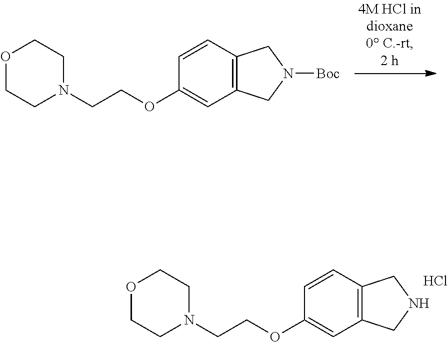 [SMILES: CC(C)(C)OC(=O)N1Cc2ccc(OCCN3CCOCC3)cc2C1 ; c1cc2c(cc1OCCN1CCOCC1)CNC2 ; Cl]

The title compound was prepared from tert-butyl 5-(2-morpholinoethoxy) isoindoline-2-carboxylate (step 1) and 4M HCl in dioxane analogously to Intermediate YD step 2.

¹H NMR (DMSO-d6, 400 MHz): δ 1.33-1.63 (m, 4H), 3.56 (d, J=8.0 Hz, 2H), 3.83 (t, J=12.0 Hz, J=23.2 Hz, 2H), 3.95 (d, J=11.6 Hz, 2H), 4.41-4.56 (m, 6H), 7.06 (d, J=2.0 Hz, 1H), 6.92-7.01 (m, 1H), 7.24-7.35 (m, 1H), 9.94 (s, 1H).

LCMS (Method N): 1.554 min, MS: ES+ 249.23 (M+1).

### Intermediate YX: 5-(2-(4-Methylpiperazin-1-yl) ethoxy) isoindoline hydrochloride

### Step 1: tert-Butyl 5-(2-(4-methyl piperazin-1-yl) ethoxy) isoindoline-2-carboxylate

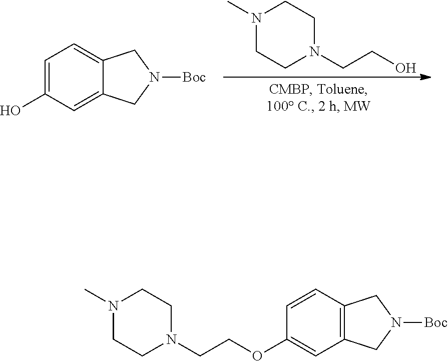

The title compound was prepared from tert-butyl 5-hydroxyisoindoline-2-carboxylate (Intermediate V step 2) and 2-(4-methylpiperazin-1-yl) ethan-1-ol analogously to Intermediate YD step 1.

¹H NMR (DMSO-d6, 400 MHz): δ 1.31 (s, 9H), 1.55-1.62 (m, 4H), 2.14 (s, 3H), 2.31-2.51 (m, 3H), 2.66 (t, J=6.0 Hz, J=11.6 Hz, 3H), 4.04 (t, J=4.4 Hz, J=10.0 Hz, 2H), 4.51 (t, J=12.4 Hz, 4H), 6.84 (d, J=8.4 Hz, 1H), 6.91 (d, J=7.6 Hz, 1H), 7.19-7.22 (m, 1H),

LCMS (Method A): 1.276 min, MS: ES+ 362.1 (M+1).

### Step 2: 5-(2-(4-Methyl piperazin-1-yl) ethoxy) isoindoline hydrochloride

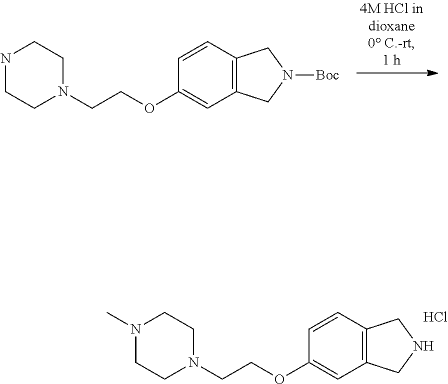 [SMILES: CC(C)(C)OC(=O)N1Cc2ccc(OCCN3CCNCC3)cc2C1 ; CN1CCN(CCOc2ccc3c(c2)CNC3)CC1 ; Cl]

The title compound was prepared from tert-butyl 5-(2-(4-methyl piperazin-1-yl) ethoxy) isoindoline-2-carboxylate carboxylate (step 1) and 4M HCl in dioxane analogously to Intermediate YD step 2.

¹H NMR (DMSO-d6, 400 MHz): δ 2.82 (s, 3H), 3.50-3.75 (m, 8H), 4.41-4.47 (m, 6H), 6.99-7.02 (m, 1H), 7.07 (s, 1H), 7.33 (d, J=8.4 Hz, 1H), 10.08 (s, 1H).

LCMS (Method A): 0.309 min, MS: ES+ 261.98 (M+1).

### Intermediate YY: N-Cyclopentyl-6-(2-(dimethyl amino) ethoxy)-1,2,3,4-tetrahydroisoquinolin-8-amine hydrochloride

### Step 1: tert-Butyl 8-(cyclopentyl amino)-6-(2-(dimethyl amino) ethoxy)-3,4-dihydroisoquinoline-2(1H)-carboxylate

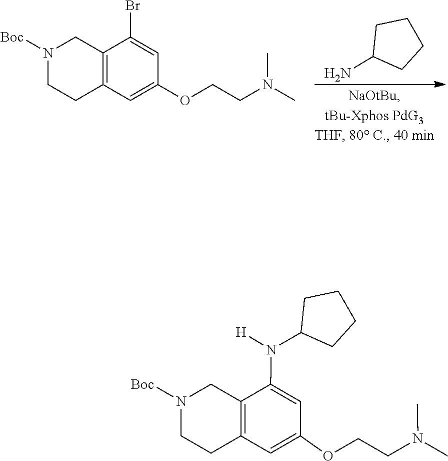 [SMILES: CN(C)CCOc1cc(Br)c2c(c1)CCN(C(=O)OC(C)(C)C)C2 ; NC1CCCC1 ; CN(C)CCOc1cc2c(c(NC3CCCC3)c1)CN(C(=O)OC(C)(C)C)CC2]

The title compound was prepared from tert-butyl 8-bromo-6-(2-(dimethylamino)ethoxy)-3,4-dihydroisoquinoline-2(1H)-carboxylate (Intermediate M step 1) and cyclopentamine (CAS: 1003-03-8) analogously to Intermediate M step 2.

¹H NMR (DMSO-d6, 400 MHz): δ 1.43 (s, 9H), 1.66-1.68 (m, 2H), 1.89-1.95 (m, 2H), 2.24 (s, 6H), 2.63-2.67 (m, 4H), 3.17 (d, J=4.4 Hz, 2H), 3.48 (t, J=4 Hz, 2H), 3.70-3.73 (m, 1H), 3.97 (t, J=5.6 Hz, 2H), 4.11-4.18 (m, 3H), 4.61 (bs, 1H).

LCMS (Method N): 1.451 min, MS: ES+ 404.2 (M+1).

### Step 2: N-Cyclopentyl-6-(2-(dimethyl amino) ethoxy)-1,2,3,4-tetrahydroisoquinolin-8-amine hydrochloride

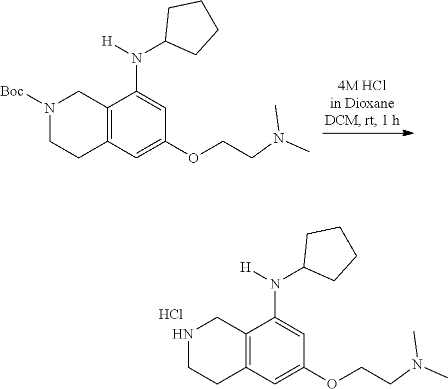 [SMILES: CN(C)CCOc1cc2c(c(NC3CCCC3)c1)CN(C(=O)OC(C)(C)C)CC2 ; Cl ; CN(C)CCOc1cc2c(c(NC3CCCC3)c1)CNCC2]

The title compound was prepared from tert-butyl 8-(cyclopentyl amino)-6-(2-(dimethyl amino) ethoxy)-3,4-dihydroisoquinoline-2(1H)-carboxylate (step 1) and 4M HCl in dioxane analogously to Intermediate M step 3.

¹H NMR (DMSO-d6, 400 MHz): δ 1.92 (d, J=4.4 Hz, 2H), 2.81 (s, 6H), 2.88-2.91 (m, 2H), 3.25-3.25 (s, 2H), 3.45 (d, J=4 Hz, 2H), 3.57 (s, 5H), 3.72-3.93 (m, 3H), 4.29-4.31 (m, 2H), 6.19 (s, 2H), 9.61 (s, 2H), 10.52 (s, 1H).

LCMS (Method N): 1.738 min, MS: ES+ 304.4 (M+1).

### Intermediate YZ: 6-(2-(Dimethylamino)ethoxy)-N-methyl-1,2,3,4-tetrahydroisoquinolin-8-amine hydrochloride

### Step 1: tert-Butyl 6-(2-(dimethylamino)ethoxy)-8-(methylamino)-3,4-dihydroisoquinoline-2(1H)-carboxylate

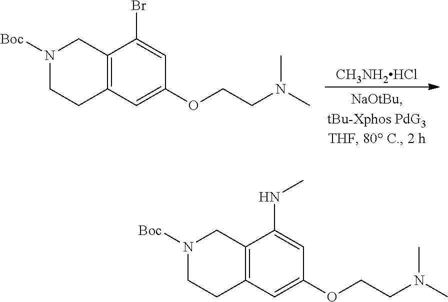

The title compound was prepared from tert-butyl 8-bromo-6-(2-(dimethylamino)ethoxy)-3,4-dihydroisoquinoline-2(1H)-carboxylate (Intermediate M step 1) and methylamine HCl (CAS: 593-51-1) analogously to Intermediate M step 2.

¹H NMR (DMSO-d6, 400 MHz): δ 1.43 (s, 9H), 2.16 (s, 6H), 2.59-2.68 (m, 5H), 2.73 (t, J=5.2 Hz, J=11.2 Hz, 1H), 2.89 (s, 1H), 3.47-3.52 (m, 2H), 3.96-4.16 (m, 2H), 4.15 (s, 1H), 4.41 (s, 1H), 5.14 (s, 1H), 5.91 (d, J=1.6 Hz, 1H), 5.99 (s, 1H).

LCMS (Method D): 6.55 min, MS: ES+ 350.3 (M+1).

### Step 2: 6-(2-(Dimethylamino)ethoxy)-N-methyl-1,2,3,4-tetrahydroisoquinolin-8-amine hydrochloride

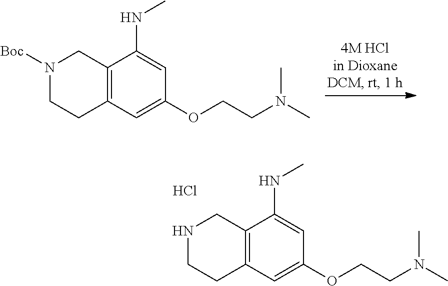

The title compound was prepared from tert-butyl 6-(2-(dimethylamino)ethoxy)-8-(methylamino)-3,4-dihydroisoquinoline-2(1H)-carboxylate (step 1) and 4M HCl in dioxane analogously to Intermediate M step 3.

LCMS (Method B): 1.454 min, MS: 250.3 (M+1)

### Intermediate ZA: 2, 6-Dihydroxy-4-(trifluoromethyl) benzoic acid

### Step 1: 3-Amino-5-(trifluoromethyl) phenol

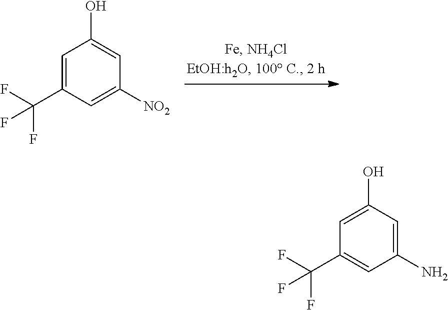 [SMILES: O=[N+]([O-])c1cc(O)cc(C(F)(F)F)c1 ; Nc1cc(O)cc(C(F)(F)F)c1]

To a stirred solution of 3-nitro-5-(trifluoromethyl)phenol (10.0 g, 48.3 mmol, 1.0 eq.) in EtOH (100 mL) were added Fe powder (13.28 g, 241.5 mmol, 5.0 eq.) and NH₄Cl (29.12 g, 724.5 mmol, 15.0 eq) at room temperature. The reaction mixture was heated to 90° C. temperature for 2 h. The resulting mixture was filtered through Celite® and the filtrate was distilled. The resulting material was diluted with water (200 mL) and extracted with EtOAc (2×100 mL). The combined organic extracts were dried over Na₂SO₄, filtered, and concentrated under vacuum. The crude material was triturated by n-pentane (3×100 mL) followed by high vacuum drying yielding 3-amino-5-(trifluoromethyl) phenol as a brown solid (8.0 g, Yield: 93.56%).

¹H NMR (DMSO-d6, 400 MHz): δ 3.86 (bs, 2H), 4.96 (bs, 1H), 6.31 (s, 1H), 6.49 (d, J=16 Hz, 2H).

LCMS (Method N): 1.325 min, MS: ES+ 178.02 (M+1).

### Step 2: 5-(Trifluoromethyl) benzene-1, 3-diol

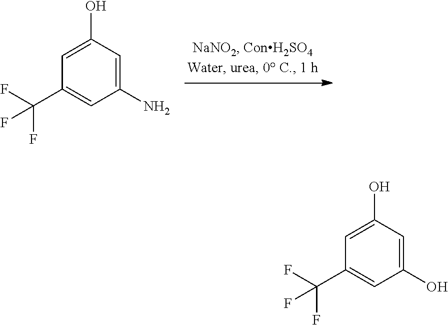 [SMILES: Nc1cc(O)cc(C(F)(F)F)c1 ; Oc1cc(O)cc(C(F)(F)F)c1]

To a stirred solution of 3-amino-5-(trifluoromethyl) phenol (step 1) (8.0 g, 4.51 mmol, 1 eq.) in water (28 mL) was added dropwise conc. H₂SO₄ (32 mL) at 0° C. A solution of sodium nitrite (3.0 g) in water (16 mL) was slowly added to the mixture at 0° C. After 15 minutes, the excess nitrous acid was removed by portion wise addition of urea. The cooled solution was added to a refluxing saturated solution of copper sulphate (200 mL) and then allowed to cool to room temperature. The mixture was extracted with ether (4×50 mL) and the combined organic extracts were dried over MgSO4, filtered, and concentrated under vacuum. The crude material was purified by chromatography on silica (eluting with 20% EtOAc in Hexane) and dried material under high vacuum yielding 5-(trifluoromethyl) benzene-1, 3-diol as a red sticky solid (3.2 g, Yield: 39.8%)

¹H NMR (DMSO-d6, 400 MHz): δ 5.25 (bs, 2H), 6.68 (t, J=2 Hz, J=4.4 Hz, 1H), 6.68 (d, J=2 Hz, 2H).

### Step 3: 2, 6-Dihydroxy-4-(trifluoromethyl) benzoic acid

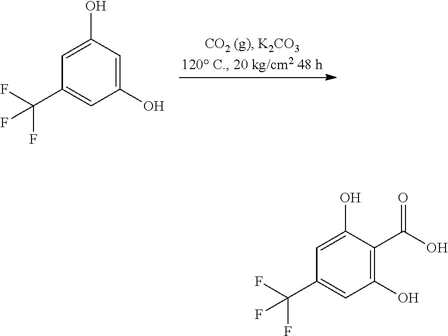 [SMILES: Oc1cc(O)cc(C(F)(F)F)c1 ; O=C(O)c1c(O)cc(C(F)(F)F)cc1O]

5-(Trifluoromethyl) benzene-1, 3-diol (step 1) (1.0 g, 5.61 mmol, 1 eq.) and K₂CO₃ (5.32 g, 5.61 mmol, 7.0 eq.) were placed under a pressure of 20 kg/cm² of CO₂ (g) in an Autoclave at room temperature. The reaction mixture was heated to 120° C. for 7 days. The resulting mixture was diluted with ice cold water (100 mL), acidified with 2N HCl (pH~6) and extracted with EtOAc (3×100 mL). The combined organic extracts were dried over Na₂SO₄, filtered and concentrated under vacuum. The crude material was triturated with n-pentane (3×100 mL) followed by high vacuum drying yielding 2, 6-dihydroxy-4-(trifluoromethyl) benzoic acid (0.8 g, Yield: 48.11%).

¹H NMR (DMSO-d6, 400 MHz): δ 6.40 (s, 2H).

LCMS (Method N): 1.369 min, MS: ES+ 220.88 (M−1).

### Intermediate ZB: (S)-6-((Dimethylamino)methyl)-N-(tetrahydrofuran-3-yl)isoindolin-4-amine hydrochloride

### Step 1: 3-Bromo-4,5-dimethylbenzoic acid

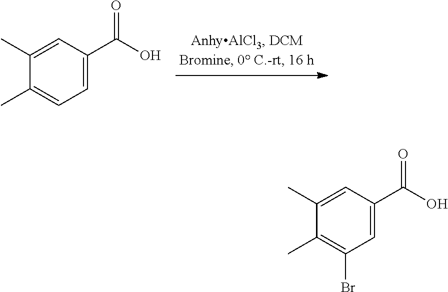

To a stirred solution of 3-bromo-4,5-dimethylphenol (5 g, 33.33 mmol, 1.0 eq.) (CAS: 619-04-5) in DCM (50 mL) was added portion wise AlCl₃ (6.64 g, 49.99 mmol, 1.5 eq.) and the mixture was stirred at room temperature for 30 min. The mixture was cooled to 0° C. and treated dropwise with bromine (25 mL). The reaction mixture was stirred at room temperature for 16 h. The resulting mixture was poured into cold water (200 mL) and extracted with DCM (2×500 mL). The combined organic extracts were dried over Na₂SO₄, filtered, and concentrated under vacuum yielding 3-bromo-4,5-dimethylbenzoic acid as a light orange solid (7.0 g, Yield: 91.7%).

¹H NMR (DMSO-d6, 400 MHz): δ 2.37 (s, 6H), 7.74 (s, 1H), 7.91 (s, 1H), 13.12 (s, 1H).

LCMS (Method A): 1.864 min, MS: ES+ 227.0 (M−1).

### Step 2: Methyl 3-bromo-4,5-dim ethyl benzoate

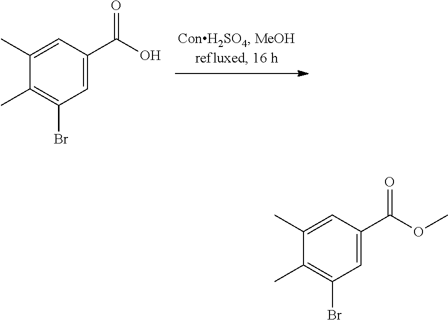

Performed in 2 parallel batches, each of 7 g scale: To a stirred solution of 3-bromo-4,5-dimethylbenzoic acid (step 1) (7.0 g, 30.56 mmol, 1.0 eq.) in MeOH (70 mL) was added dropwise conc. H₂SO₄ (3.89 g, 39.73 mmol, 1.3 eq.) and the reaction mixture was heated to 70° C. and stirred for 16 h. The resulting mixture was diluted with water (200 mL) and extracted with EtOAc (2×125 mL). The combined organic extracts were dried over Na₂SO₄, filtered, and concentrated under vacuum. The crude material was purified by chromatography on silica (eluting with 14% EtOAc in hexane) to yield methyl 3-bromo-4,5-dimethylbenzoate (10.0 g, Yield: 67.34%).

¹H NMR (DMSO-d6, 400 MHz): δ 2.35 (s, 6H), 3.83 (s, 3H), 7.72 (s, 1H), 7.89 (s, 1H).

### Step 3: Methyl 3-bromo-4,5-bis(bromomethyl)benzoate

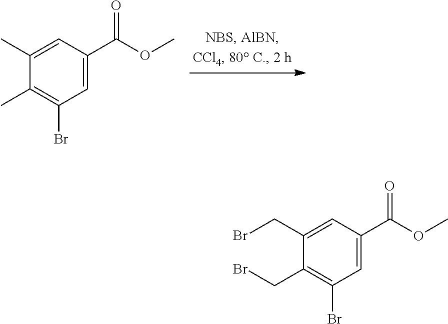

To a stirred solution of methyl 3-bromo-4,5-dim ethyl benzoate (step 2) (10.0 g, 41.15 mmol, 1.0 eq.) in CCl₄ (100 mL) were added NBS (14.65 g, 82.30 mmol, 2.0 eq.) and AIBN (0.67 g, 4.115 mmol, 0.1 eq.) at room temperature. The reaction mixture was heated to 80° C. for 2 h. The resulting mixture was diluted with water (200 mL) and extracted with EtOAc (2×125 mL). The combined organic extracts were dried over Na₂SO₄, filtered, and concentrated under vacuum. The crude material was purified by chromatography on silica (eluting with 2.5% EtOAc in hexane) yielding methyl 3-bromo-4,5-bis(bromomethyl)benzoate (10.0 g, Yield: 60.75%)

¹H NMR (DMSO-d6, 400 MHz): δ 3.89 (s, 3H), 4.89 (s, 2H), 4.95 (s, 2H), 8.11 (d, J=2.4 Hz, 2H).

### Step 4: 4-Bromo-6-methoxy-2-(4-methoxybenzyl)isoindoline

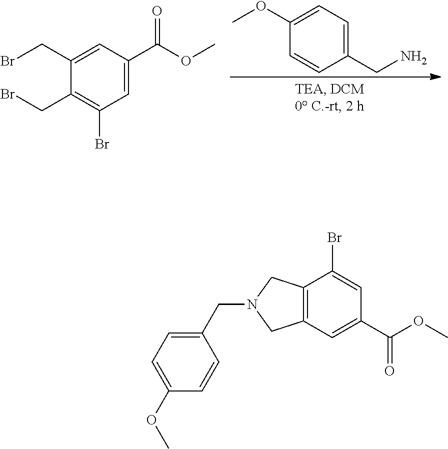

To a stirred solution of methyl 3-bromo-4,5-bis(bromomethyl)benzoate (step 3) (10.0 g, 25.00 mmol, 1.0 eq.) in DCM (100 mL) was added TEA (7.57 g, 75.00 mmol, 3.0 eq.) at room temperature and stirred for 15 min. 4-Methoxy benzyl amine (6.85 g, 50 mmol, 2.0 eq.) was added dropwise at 0° C. and the reaction mixture was stirred for 2 h. The resulting mixture was diluted with water (250 mL) and extracted with DCM (2×125 mL). The combined organic extracts were dried over Na₂SO₄, filtered and concentrated under vacuum. The crude material was purified by chromatography on silica (eluting with 14% EtOAc in hexane) yielding 4-bromo-6-methoxy-2-(4-methoxybenzyl)isoindoline (3.0 g, Yield: 42.55%).

¹H NMR (DMSO-d6, 400 MHz): δ 3.73-3.75 (m, 3H), 3.80 (s, 2H), 3.85 (s, 5H), 3.98 (s, 2H), 6.91 (d, J=8.8 Hz, 2H), 7.28 (d, J=8.4 Hz, 2H), 7.81 (s, 1H), 7.90 (s, 1H)

LCMS (Method A): 1.417 min, MS: ES+ 375.9, 377.8 (M & M+2)

### Step 5: Methyl 7-bromoisoindoline-5-carboxylate hydrochloride

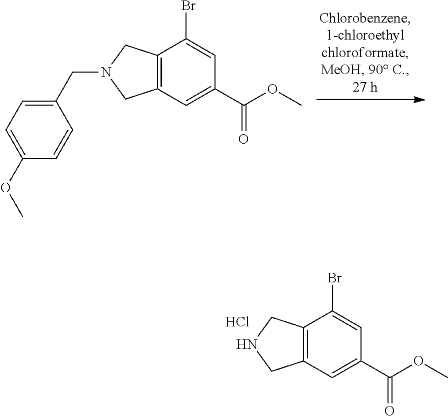 [SMILES: COC(=O)c1cc(Br)c2c(c1)CN(Cc1ccc(OC)cc1)C2 ; Cl ; COC(=O)c1cc(Br)c2c(c1)CNC2]

The title compound was prepared from 4-bromo-6-methoxy-2-(4-methoxybenzyl) isoindoline (step 4) and 1-chloroethyl chloroformate analogously to Intermediate YK step 1e.

¹H NMR (DMSO-d6, 400 MHz): δ 3.87 (s, 3H), 4.52-4.66 (m, 4H), 8.02 (d, J=12.8 Hz, 2H), 10.6 (s, 1H).

LCMS (Method A): 0.865 min, MS: ES+ 255.7 & 257.7 (M & M+2).

### Step 6: 2-(tert-Butyl) 5-methyl 7-bromoisoindoline-2,5-dicarboxylate

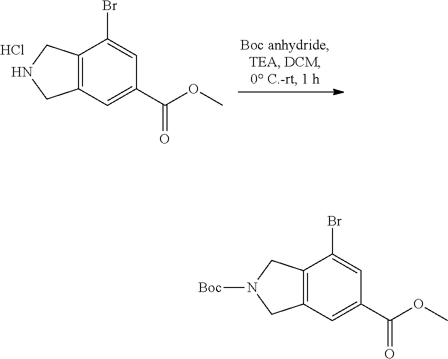 [SMILES: Cl ; COC(=O)c1cc(Br)c2c(c1)CNC2 ; COC(=O)c1cc(Br)c2c(c1)CN(C(=O)OC(C)(C)C)C2]

The title compound was prepared from methyl 7-bromoisoindoline-5-carboxylate hydrochloride (step 5) and Boc-anhydride analogously to Intermediate YK step 1f.

¹H NMR (DMSO-d6, 400 MHz): δ 1.46 (s, 9H), 3.87 (s, 3H), 4.58 (d, J=9.2 Hz, 2H), 4.74 (d, J=9.2 Hz, 2H), 7.93 (d, J=8.8 Hz, 1H), 7.99 (s, 1H).

LCMS (Method A): 2.413 min, MS: ES− 299.6 (M−56).

### Step 7: 2-(tert-Butyl) 5-methyl (S)-7-((tetrahydrofuran-3-yl)amino)isoindoline-2,5-dicarboxylate

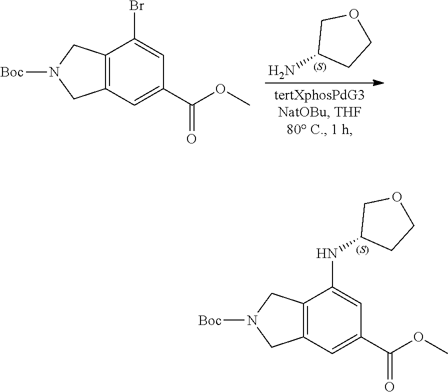

The title compound was prepared from 2-(tert-butyl) 5-methyl 7-bromoisoindoline-2,5-dicarboxylate (step 1) and (S)-tetrahydrofuran-3-amine (CAS: 104530-79-2) analogously to Intermediate B1-1 step 1.

¹H NMR (DMSO-d6, 400 MHz): δ 1.45-1.47 (m, 9H), 1.52 (s, 1H), 1.84-1.86 (m, 1H), 2.15-2.17 (m, 1H), 3.59-3.64 (m, 1H), 3.71-3.76 (m, 1H), 3.81-3.92 (m, 4H), 4.06 (s, 1H), 4.45 (s, 2H), 4.55 (d, J=10.4 Hz, 2H), 5.66-5.73 (m, 1H), 6.92-6.99 (m, 1H), 7.10-7.19 (m, 1H).

LCMS (Method A): 2.022 min, MS: ES+ 306.9 (M−56).

### Step 8: tert-Butyl (S)-6-(hydroxymethyl)-4-((tetrahydrofuran-3-yl)amino)isoindoline-2-carboxylate

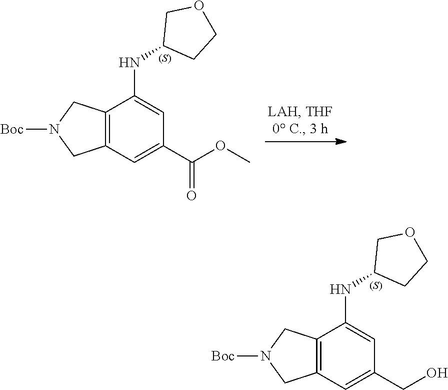

The title compound was prepared from 2-(tert-butyl) 5-methyl (S)-7-((tetrahydrofuran-3-yl)amino)isoindoline-2,5-dicarboxylate (step 7) and LAH (1M in THF) analogously to Intermediate YF step 1.

¹H NMR (DMSO-d6, 400 MHz): δ 1.42-1.47 (m, 9H), 1.82-1.86 (m, 1H), 2.14-2.19 (m, 1H), 3.54-3.59 (m, 1H), 3.71-3.74 (m, 1H), 3.80-3.83 (m, 1H), 3.90 (t, J=7.2 Hz, 1H), 4.00-4.02 (m, 1H), 4.38-4.41 (m, 4H), 4.48 (d, J=10.4 Hz, 2H), 5.07 (t, J=6 Hz, 1H), 5.32-5.33 (m, 1H), 6.41 (s, 1H), 6.51 (d, J=11.6 Hz, 1H).

LCMS (Method A): 1.557 min, MS: ES− 278.9 (M−56).

### Step 9: tert-Butyl (S)-6-formyl-4-((tetrahydrofuran-3-yl)amino)isoindoline-2-carboxylate

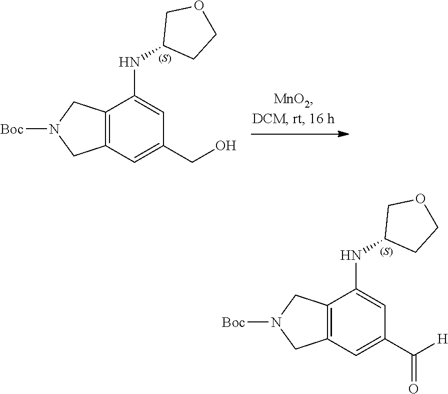

To a stirred solution of tert-butyl (S)-6-(hydroxymethyl)-4-((tetrahydrofuran-3-yl)amino)isoindoline-2-carboxylate (step 8) (0.18 g, 0.538 mmol, 1.0 eq) in DCM (1.8 mL) was added activated MnO₂ (0.9 g, 200% w/w) (Sigma Aldrich) at room temperature. The reaction mixture was allowed to stir at room temperature for 16 h. The resulting mixture was filtered through Celite® (filter material), washing through with DCM (30 mL). The filtrate was concentrated under high vacuum yielding tert-butyl (S)-6-formyl-4-((tetrahydrofuran-3-yl)amino)isoindoline-2-carboxylate (0.16 g, Yield: 64.51%).

¹H NMR (DMSO-d6, 400 MHz): δ 1.43-1.48 (m, 9H), 1.84-1.88 (m, 1H), 2.18-2.24 (m, 1H), 3.60-3.64 (m, 1H), 3.72-3.77 (m, 1H), 3.82-3.87 (m, 1H), 3.92-3.95 (m, 1H), 4.11-4.12 (m, 1H), 4.47-4.48 (m, 2H), 4.58 (d, J=10.8 Hz, 2H), 5.76-5.80 (m, 1H), 6.95 (m, 1H), 7.12 (d, J=10.4 Hz, 1H), 9.89 (d, J=1.6 Hz, 1H).

LCMS (Method A): 1.863 min, MS: ES− 276.8 (M−56).

### Step 10: tert-Butyl (S)-6-((dimethylamino)methyl)-4-((tetrahydrofuran-3-yl)amino)isoindoline-2-carboxylate

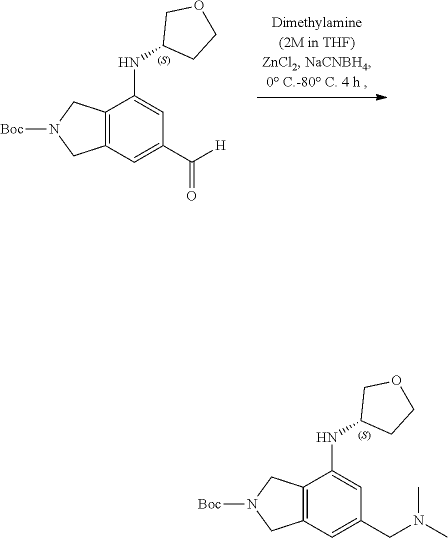

The title compound was prepared from tert-butyl (S)-6-formyl-4-((tetrahydrofuran-3-yl)amino)isoindoline-2-carboxylate (step 10) and dimethyl amine (2M in THF) analogously to Intermediate RA step 2.

LCMS (Method A): 1.222 min, MS: ES+ 362.1 (M+1).

### Step 11: (S)-6-((Dimethylamino)methyl)-N-(tetrahydrofuran-3-yl)isoindolin-4-amine hydrochloride

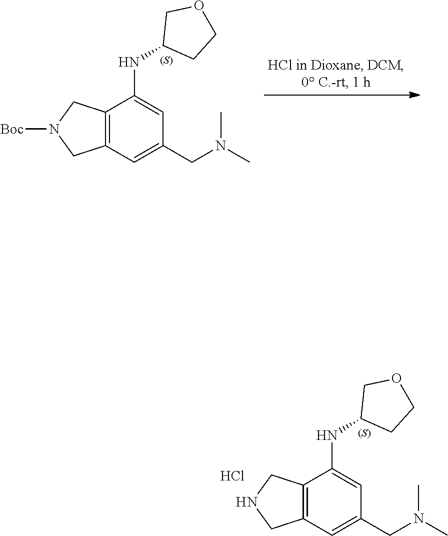

The title compound was prepared from tert-butyl (S)-6-((dimethylamino)methyl)-4-((tetrahydrofuran-3-yl)amino)isoindoline-2-carboxylate (step 10) and 4M HCl in dioxane analogously to Intermediate R step 5.

¹H NMR (DMSO-d6, 400 MHz): δ 1.81-1.89 (m, 1H), 2.22-2.29 (m, 1H), 2.66-2.67 (m, 6H), 3.56-3.59 (m, 2H), 3.70-3.75 (m, 1H), 3.82-3.88 (m, 1H), 3.95-3.99 (m, 1H), 4.06-4.08 (m, 1H), 4.18 (d, J=5.6 Hz, 2H), 4.33 (s, 2H), 4.42 (s, 2H), 6.72-6.87 (m, 2H), 10.11 (s, 1H).

LCMS (Method F): 1.48 min, MS ES+: 262.4 (M+1).

### Intermediate ZC: (S)-1-Methyl-3-((1,2,3,4-tetrahydroisoquinolin-8-yl)amino)pyrrolidin-2-one hydrochloride

### Step 1: tert-Butyl(S)-8-((1-methyl-2-oxopyrrolidin-3-yl)amino)-3,4-dihydroisoquinoline-2(1H)-carboxylate

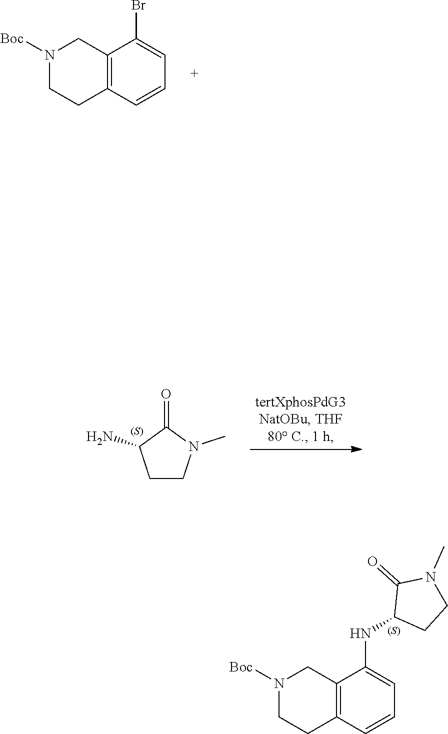

The title compound was prepared from tert-butyl 8-bromo-3,4-dihydroisoquinoline-2(1H)-carboxylate (CAS: 893566-75-1) and (S)-3-amino-1-methylpyrrolidin-2-one (Intermediate W step 4) analogously to Intermediate B1-1 step 1.

¹H NMR (DMSO-d6, 400 MHz): δ 1.47 (bs, 9H), 1.75-1.85 (m, 1H), 2.69 (t, J=5.2 Hz, 2H), 2.80 (s, 3H), 3.34 (s, 1H), 3.45-4.09 (m, 2H), 4.09-4.13 (m, 1H), 4.26 (bs, 2H), 4.90 (m, 1H), 6.47 (d, J=7.6 Hz, 1H), 6.55 (d, J=8 Hz, 1H), 6.99 (t, J=7.6 Hz, 1H).

LCMS (Method A): 1.906 min, MS: ES+ 246 (M−56).

### Step 2: (S)-1-Methyl-3-((1,2,3,4-tetrahydroisoquinolin-8-yl)amino)pyrrolidin-2-one hydrochloride

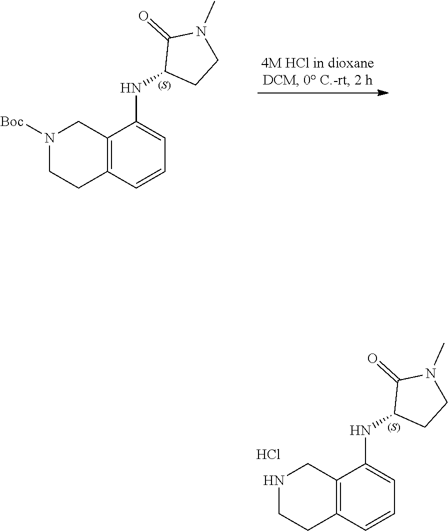

The title compound was prepared from tert-butyl (S)-8-((1-methyl-2-oxopyrrolidin-3-yl)amino)-3,4-dihydroisoquinoline-2(1H)-carboxylate carboxylate (step 1) and 4M HCl in dioxane analogously to Intermediate YM step 2.

¹H NMR (DMSO-d6, 400 MHz): δ 1.76-1.92 (m, 1H), 2.79 (s, 3H), 2.93 (t, J=6 Hz, 2H), 3.27-3.35 (m, 4H), 3.57 (s, 3H), 3.95-3.96 (bs, 2H), 4.19 (t, J=8.8 Hz, 1H), 5.77 (s, 1H), 6.32 (bs, 2H), 6.51 (d, J=7.6 Hz, 1H), 6.63 (d, J=8 Hz, 1H), 7.06 (t, J=8 Hz, 1H), 9.65 (bs, 2H)·HCl salt.

LCMS (Method F): 1.56 min, MS: ES+ 246 (M+1).

### Intermediate ZD: (R)-1-Methyl-3-((1,2,3,4-tetrahydroisoquinolin-8-yl)amino)pyrrolidin-2-one hydrochloride

### Step 1: tert-Butyl(R)-8-((1-methyl-2-oxopyrrolidin-3-yl)amino)-3,4-dihydroisoquinoline-2(1H)-carboxylate

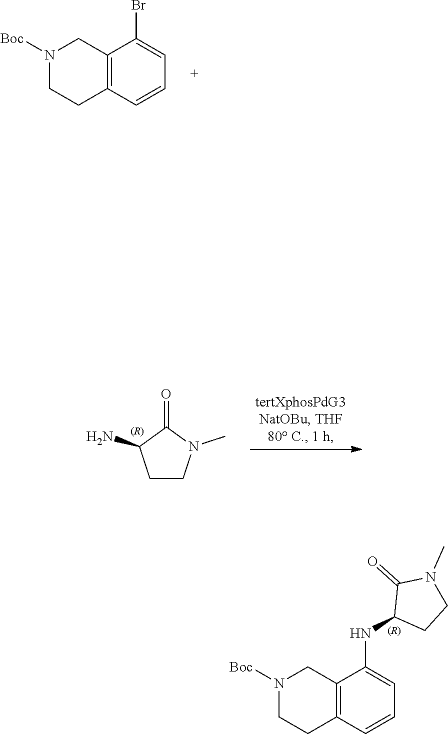

The title compound was prepared from tert-butyl 8-bromo-3,4-dihydroisoquinoline-2(1H)-carboxylate (CAS: 893566-75-1) and (R)-3-amino-1-methylpyrrolidin-2-one (Intermediate X step 3) analogously to Intermediate B1-1 step 1.

¹H NMR (DMSO-d6, 400 MHz): δ 1.44 (bs, 9H), 1.77-1.85 (m, 1H), 2.70 (t, J=5.6 Hz, 2H), 2.80 (s, 3H), 3.35 (s, 1H), 3.49-3.56 (m, 2H), 4.07-4.13 (m, 1H), 4.26 (bs, 2H), 4.90 (m, 1H), 6.47 (d, J=7.6 Hz, 1H), 6.55 (d, J=8 Hz, 1H), 6.99 (t, J=8 Hz, 1H).

LCMS (Method A): 1.901 min, MS: ES+ 246 (M−100).

### Step 2: (R)-1-Methyl-3-((1,2,3,4-tetrahydroisoquinolin-8-yl)amino)pyrrolidin-2-one hydrochloride

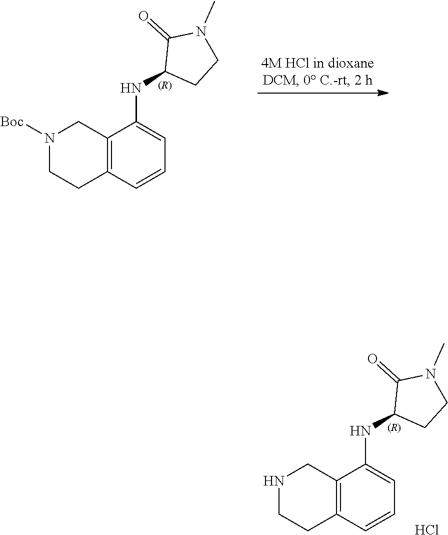

The title compound was prepared from tert-butyl (R)-8-((1-methyl-2-oxopyrrolidin-3-yl)amino)-3,4-dihydroisoquinoline-2(1H)-carboxylate (step 1) and 4M HCl in dioxane analogously to Intermediate YM step 2.

¹H NMR (DMSO-d6, 400 MHz): δ 1.74-1.84 (m, 2H), 2.79 (s, 3H), 2.93 (t, J=5.6 Hz, 2H), 3.27-3.35 (m, 4H), 3.57 (s, 2H), 3.95-3.96 (t, J=4.8 Hz, 2H), 4.17 (t, J=8.8 Hz, 1H), 6.51 (d, J=7.6 Hz, 1H), 6.62 (d, J=8.4 Hz, 1H), 6.93 (bs, 2H), 7.06 (t, J=7.6 Hz, 1H), 9.65 (bs, 2H),

LCMS (Method F): 1.56 min, MS: ES+ 246 (M+1).

### Intermediate ZE: 3-(Isoindolin-4-ylamino)dihydrofuran-2(3H)-one hydrochloride

### Step 1: tert-Butyl 4-((2-oxotetrahydrofuran-3-yl)amino)isoindoline-2-carboxylate

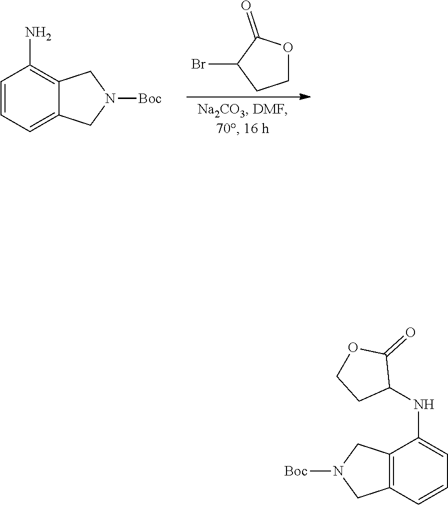 [SMILES: CC(C)(C)OC(=O)N1Cc2cccc(N)c2C1 ; O=C1OCCC1Br ; CC(C)(C)OC(=O)N1Cc2cccc(NC3CCOC3=O)c2C1]

To a stirred solution of tert-butyl 4-aminoisoindoline-2-carboxylate (0.5 g, 2.13 mmol, 1.0 eq) in DMF (5 mL) were added 3-bromodihydrofuran-2(3H)-one (CAS: 5061-21-2) (0.2 mL, 2.13 mmol, 1.0 eq) and Na₂CO₃ (1.1 g, 10.68 mmol, 5 eq) at room temperature and the reaction mixture was heated to 70° C. for 16 h. The resulting mixture was cooled to room temperature, diluted with water (20 mL) and extracted with EtOAc (3×30 mL). The combined organic extracts were dried over Na₂SO₄ and concentrated under vacuum. The crude material was purified using flash column chromatography (eluting product in 29% EtOAc:hexane) yielding tert-butyl 4-((2-oxotetrahydrofuran-3-yl)amino)isoindoline-2-carboxylate as a light yellow solid (0.3 g, Yield: 44.2%).

¹H NMR (DMSO-d6, 400 MHz): δ 1.46 (d, J=5.6 Hz, 9H), 2.21-2.13 (m, 1H), 3.34 (s, 1H), 4.27-4.20 (m, 1H), 4.41-4.37 (m, 3H), 4.52 (d, J=11 Hz, 2H), 4.54-4.65 (m, 1H), 5.66-5.61 (m, 1H), 6.59 (t, J=4.4 Hz, 2H), 7.08 (t, J=8 Hz, 2H).

LCMS (Method A): 1.860 min, MS: ES-218.9 (M−100).

### Step 2: 3-(Isoindolin-4-ylamino)dihydrofuran-2(3H)-one hydrochloride

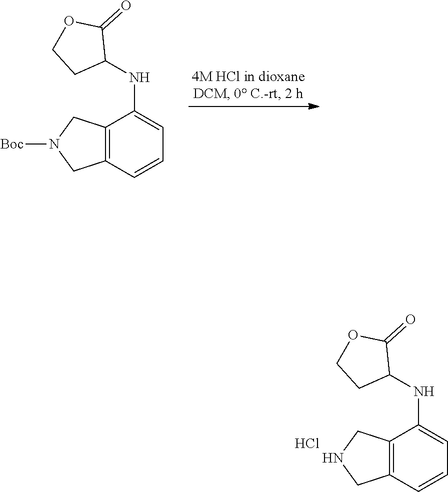 [SMILES: CC(C)(C)OC(=O)N1Cc2cccc(NC3CCOC3=O)c2C1 ; Cl ; O=C1OCCC1Nc1cccc2c1CNC2]

The title compound was prepared from tert-butyl 4-((2-oxotetrahydrofuran-3-yl)amino)isoindoline-2-carboxylate (step 1) and 4M HCl in dioxane analogously to Intermediate YM step 2.

¹H NMR (DMSO-d6, 400 MHz): δ 2.21-2.15 (m, 1H), 2.62-2.52 (m, 1H), 3.27 (s, 1H), 4.26-4.22 (m, 1H), 4.32 (t, J=5.2 Hz, 2H), 4.41 (t, J=5.6 Hz, 3H), 4.68-4.63 (m, 1H), 6.67 (t, J=8.4 Hz, 2H), 7.16 (t, J=8 Hz, 1H), 9.81 (d, J=21 Hz, 1H).

LCMS (Method B): 1.45 min, MS: ES+ 219.2 (M+1).

### Intermediate ZF: 3-((1,2,3,4-Tetrahydroisoquinolin-8-yl) amino) dihydrofuran-2(3H)-one hydrochloride

### Step 1: 1,2,3,4-Tetrahydroisoquinolin-8-amine

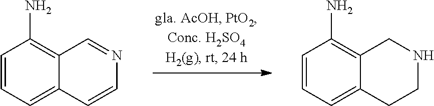 [SMILES: Nc1cccc2ccncc12 ; Nc1cccc2c1CNCC2]

To a stirred solution of isoquinolin-8-amine (CAS 23687-27-6) (5.0 g, 34.67 mmol, 1 eq.) in glacial acetic acid (75 mL) were added conc. H₂SO₄ (0.5 mL) and PtO₂ (0.5 g, 1% w/w) at room temperature. The reaction reaction was placed under H₂ pressure (25 kg/cm³) in an autoclave and stirred at room temperature for 24 h. The resulting mixture was concentrated under vacuum. The residue was treated with K₂CO₃ solution (200 mL) and extracted with 10% MeOH in DCM (5×100 mL). The combined organic extracts were dried over Na₂SO₄, filtered, and concentrated under vacuum. The crude material was triturated with diethyl ether (100 mL) and concentrated under reduced pressure yielding 1,2,3,4-tetrahydroisoquinolin-8-amine as a yellow liquid (3.6 g, Yield: 70.05%).

¹H NMR (DMSO-d6, 400 MHz): δ 2.58 (t, J=5.6 Hz, 2H), 2.87 (t, J=5.6 Hz, 2H), 3.57 (s, 2H), 4.68 (s, 2H), 6.28 (d, J=7.6 Hz, 1H), 6.41 (d, J=8 Hz, 1H), 6.79 (t, J=7.6 Hz, 1H).

LCMS (Method A): 0.194 min, MS: ES+ 149.2 (M+1).

### Step 2: tert-Butyl 8-amino-3,4-dihydroisoquinoline-2(1H)-carboxylate

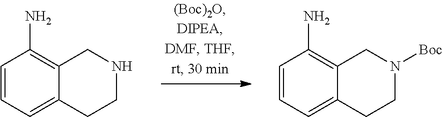 [SMILES: Nc1cccc2c1CNCC2 ; CC(C)(C)OC(=O)N1CCc2cccc(N)c2C1]

The title compound was prepared from 1,2,3,4-tetrahydroisoquinolin-8-amine (step 1), Boc anhydride and DIPEA analogously to Intermediate N step 1.

¹H NMR (DMSO-d6, 400 MHz): δ 1.43 (s, 9H), 2.65 (t, J=5.6 Hz, 2H), 3.50 (t, J=6 Hz, 2H), 4.22 (s, 2H), 4.87 (s, 2H), 6.35 (d, J=7.2 Hz, 1H), 6.49 (d, J=8 Hz, 1H), 6.85 (t, J=7.6 Hz, 1H).

LCMS (Method A): 1.746 min, MS: ES+ 192.93 (M−56)

### Step 3: tert-Butyl 8-((2-oxotetrahydrofuran-3-yl) amino)-3,4-dihydroisoquinoline-2(1H)-carboxylate

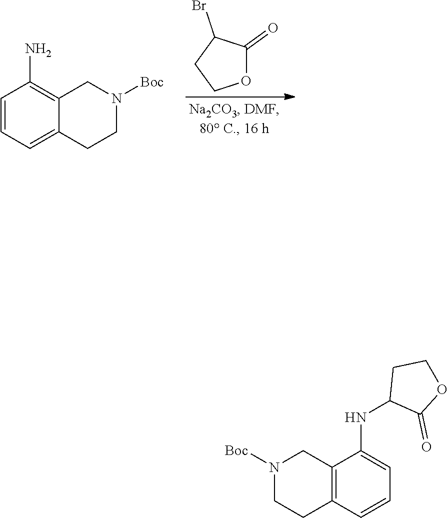 [SMILES: CC(C)(C)OC(=O)N1CCc2cccc(N)c2C1 ; O=C1OCCC1Br ; CC(C)(C)OC(=O)N1CCc2cccc(NC3CCOC3=O)c2C1]

The title compound was prepared from tert-butyl 8-amino-3,4-dihydroisoquinoline-2(1H)-carboxylate (step 2) and 3-bromodihydrofuran-2(3H)-one (CAS 5061-21-2) analogously to Intermediate ZE step 1.

¹H NMR (DMSO-d6, 400 MHz): δ 1.45 (s, 9H), 2.16-2.22 (m, 1H), 2.55-2.60 (m, 1H), 2.69 (t, J=5.6 Hz, 2H), 3.17 (d, J=5.2 Hz, 1H), 3.50-3.57 (m, 2H), 4.21-4.28 (m, 3H), 4.37-4.41 (m, 1H), 4.58 (t, J=2 Hz, 1H), 5.28 (bs, 1H), 6.48 (d, J=7.6 Hz, 1H), 6.59 (d, J=8.4 Hz, 1H), 6.99 (t, J=7.6 Hz, 1H),

LCMS (Method A): 2.008 min, MS: ES+ 232.98 (M+1).

### Step 4: 3-((1,2,3,4-Tetrahydroisoquinolin-8-yl) amino) dihydrofuran-2(3H)-one hydrochloride

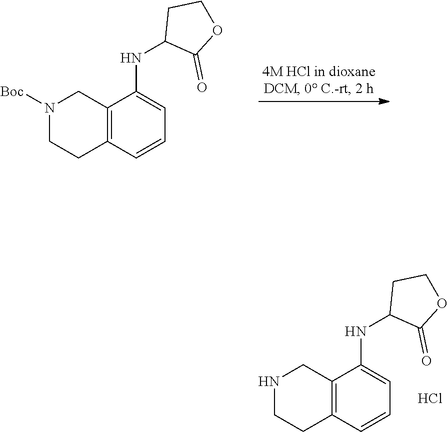 [SMILES: CC(C)(C)OC(=O)N1CCc2cccc(NC3CCOC3=O)c2C1 ; O=C1OCCC1Nc1cccc2c1CNCC2 ; Cl]

The title compound was prepared from tert-butyl (2-(2-(1,3-dioxoisoindolin-2-yl) ethoxy) ethyl) (methyl)carbamate (step 3) and 4M HCl in dioxane analogously to Intermediate YM step 2.

¹H NMR (DMSO-d6, 400 MHz): δ 2.13-2.22 (m, 1H), 2.56-2.63 (m, 1H), 2.93 (t, J=6.4 Hz, 2H), 3.30 (bs, 2H), 3.57 (s, 1H), 3.96 (s, 2H), 4.22-4.28 (m, 1H), 4.38-4.42 (m, 1H), 4.59-4.64 (m, 2H), 6.54 (d, J=7.2 Hz, 1H), 6.66 (d, J=8 Hz, 1H), 7.08 (t, J=8 Hz, 1H), 9.52 (bs, 2H).

LCMS (Method A): 0.767 min, MS: ES+ 232.9 (M+1).

### Intermediate ZG: (S)-6-((1H-Imidazol-1-yl)methyl)-N-(tetrahydrofuran-3-yl)isoindolin-4-amine hydrochloride

### Step 1: tert-Butyl 4-bromo-6-(hydroxymethyl)isoindoline-2-carboxylate

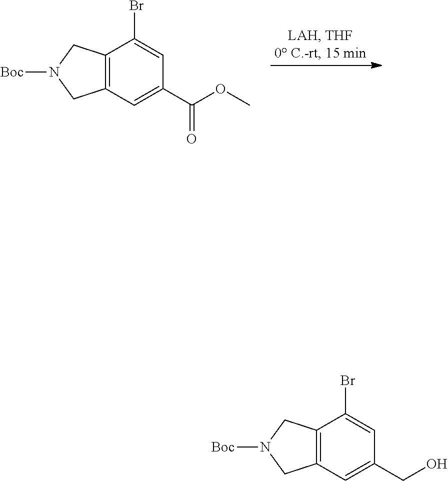 [SMILES: COC(=O)c1cc(Br)c2c(c1)CN(C(=O)OC(C)(C)C)C2 ; CC(C)(C)OC(=O)N1Cc2cc(CO)cc(Br)c2C1]

The title compound was prepared from 2-(tert-butyl) 5-methyl 7-bromoisoindoline-2,5-dicarboxylate (Intermediate ZB step 6) and LAH solution (1M in THF) analogously to Intermediate YF step 1.

¹H NMR (DMSO-d6, 400 MHz): δ 1.46 (d, J=1.2 Hz, 9H), 4.50 (t, J=5.6 Hz, 4H), 4.67 (d, J=8.4 Hz, 2H), 5.36 (t, J=6 Hz, 1H), 7.27 (d, J=9.6 Hz, 1H), 7.42 (s, 1H).

LCMS (Method A): 1.911 min, MS: ES+ 271.8 & 273.7 (M−56)

### Step 2: tert-Butyl 4-bromo-6-(bromomethyl)isoindoline-2-carboxylate

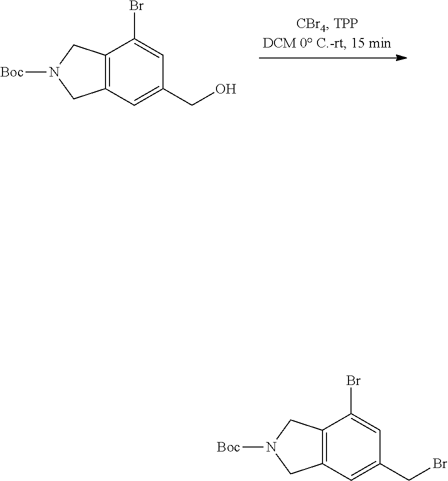 [SMILES: CC(C)(C)OC(=O)N1Cc2cc(CO)cc(Br)c2C1 ; CC(C)(C)OC(=O)N1Cc2cc(CBr)cc(Br)c2C1]

The title compound was prepared from tert-butyl 4-bromo-6-(hydroxymethyl)isoindoline-2-carboxylate (step 1), CBr₄ and triphenyl phosphine analogously to Intermediate R step 3.

¹H NMR (DMSO-d6, 400 MHz): δ 1.46 (s, 9H), 4.53 (m, 2H), 4.69 (t, J=10.8 Hz, 3H), 7.42 (d, J=8 Hz, 1H), 7.64 (s, 1H).

LCMS (Method A): 2.525 min, MS: ES+ 333.5 & 335.7 (M−56)

### Step 3: tert-Butyl 6-((1H-imidazol-1-yl) methyl)-4-bromoisoindoline-2-carboxylate

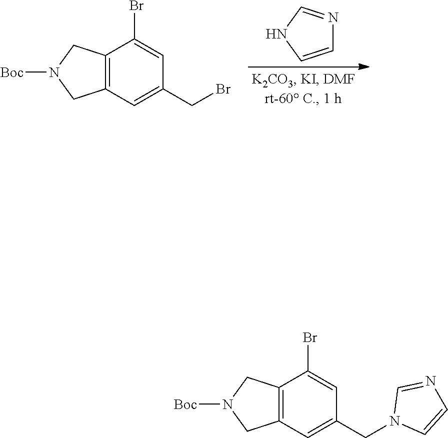 [SMILES: CC(C)(C)OC(=O)N1Cc2cc(CBr)cc(Br)c2C1 ; c1c[nH]cn1 ; CC(C)(C)OC(=O)N1Cc2cc(Cn3ccnc3)cc(Br)c2C1]

The title compound was prepared from tert-butyl 4-bromo-6-(bromomethyl)isoindoline-2-carboxylate (step 2) and 1H-imidazole analogously to Intermediate YF step 3

¹H NMR (DMSO-d6, 400 MHz): δ 1.45 (d, J=3.2 Hz, 9H), 4.49 (d, J=9.6 Hz, 2H), 4.66 (d, J=10 Hz, 2H), 5.11 (s, 2H), 6.91 (s, 1H), 7.20-7.26 (m, 2H), 7.43 (s, 1H), 7.76 (d, J=3.2 Hz, 1H).

LCMS (Method A): 1.421 min, MS: ES+ 377.8 & 379.6 (M+1)

### Step 4: tert-Butyl (S)-6-((1H-imidazol-1-yl) methyl)-4-((tetrahydrofuran-3-yl)amino)isoindoline-2-carboxylate

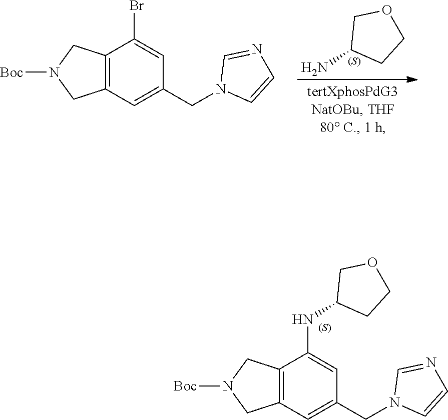

The title compound was prepared from tert-butyl 6-((1H-imidazol-1-yl) methyl)-4-bromoisoindoline-2-carboxylate (step 3) and (S)-tetrahydrofuran-3-amine analogously to Intermediate B1-1 step 1.

¹H NMR (DMSO-d6, 400 MHz): δ 1.44 (t, J=4.4 Hz, 9H), 1.80-1.83 (m, 1H), 2.13-2.16 (m, 1H), 3.17 (d, J=5.2 Hz, 1H), 3.50-3.51 (m, 1H), 3.70-3.82 (m, 2H), 3.87-3.92 (m, 2H), 4.36-4.46 (m, 4H), 5.07 (s, 2H), 5.48-5.52 (m, 1H), 6.37 (dd, J=7.6 Hz, 2H), 6.88 (s, 1H), 7.19 (s, 1H), 7.72 (s, 1H).

LCMS (Method A): 1.281 min, MS: ES+ 384.9 (M+1).

### Step 5: (S)-6-((1H-Imidazol-1-yl)methyl)-N-(tetrahydrofuran-3-yl)isoindolin-4-amine hydrochloride

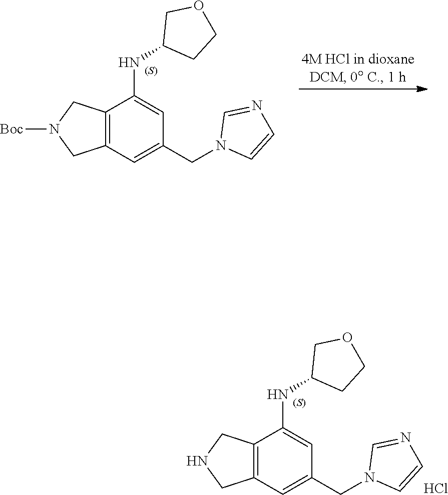

The title compound was prepared from tert-butyl (S)-6-((1H-imidazol-1-yl)methyl)-4-((tetrahydrofuran-3-yl)amino)isoindoline-2-carboxylate (step 4) and 4M HCl in dioxane analogously to Intermediate YM step 2.

LCMS (Method N): 1.703 min, MS: ES+ 285.3 (M+1).

### Intermediate ZH: (S)-6-Methoxy-N-(tetrahydrofuran-3-yl)-1,2,3,4-tetrahydroisoquinolin-8-amine hydrochloride

### Step 1a: 1-Bromo-3-(bromomethyl)-5-methoxybenzene

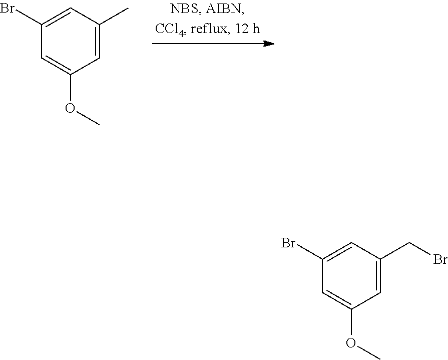

The title compound was prepared from 1-bromo-3-methoxy-5-methylbenzene (CAS: 29578-83-4) and NBS analogously to Intermediate ZB step 3.

¹H NMR (DMSO-d6, 400 MHz): δ 3.82 (s, 9H), 3.87 (s, 3H), 4.40 (s, 2H), 6.87 (t, J=1.6 Hz, J=3.6 Hz, 1H), 6.94-7.06 (m, 1H), 7.14 (t, J=1.2 Hz, J=2.8 Hz, 1H).

### Step 1b: 2-(3-Bromo-5-methoxyphenyl)acetonitrile

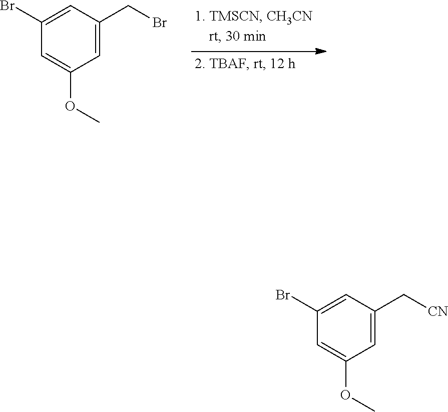

To a stirred solution of 1-bromo-3-(bromomethyl)-5-methoxybenzene (step 1a) (100 g, 358.4 mmol, 1.0 eq.) in ACN (1000 mL) was added TMSCN (35.5 g, 358.4 mmol, 1 eq.) and the mixture was stirred at room temperature for 30 mins. TBAF (93.55 g, 358.4 mmol, 1 eq.) was added and the reaction mixture was stirred at room temperature for 16 h. The mixture was dried over Na₂SO₄, filtered, and concentrated under vacuum. The crude material was purified by chromatography on silica eluting with EtOAc in hexane. The pure fractions were distilled to yield 2-(3-bromo-5-methoxyphenyl)acetonitrile (100 g, Yield: 66%).

¹H NMR (CDCl3, 400 MHz): δ 3.72 (s, 2H), 3.83 (s, 3H), 6.82 (t, J=1.2 Hz, J=2 Hz, 1H), 7.03 (t, J=2 Hz, J=4 Hz, 1H), 7.08 (t, J=0.8 Hz, J=1.6 Hz, 1H).

### Step 1c: 2-(3-Bromo-5-methoxyphenyl)ethan-1-amine

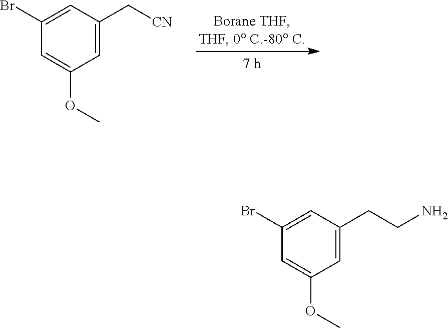

To a stirred solution of 2-(3-bromo-5-methoxyphenyl)acetonitrile (step 1 b) (60 g, 265.4 mmol, 1.0 eq.) in THF (600 mL) was cooled to 0° C. BH₃ (1M in THF) (1327 mL, 1327 mmol, 5 eq.) was added dropwise and the reaction mixture was heated to 80° C. for 6 h. The reaction was quenched with MeOH (2.5 L) and the mixture was concentrated under vacuum yielding 2-(3-bromo-5-methoxyphenyl)ethan-1-amine (180 g, Yield: 90%).

LCMS (Method A): 1.06 min, MS: ES+ 229.7 (M+1).

### Step 1 d: 8-Bromo-6-methoxy-1,2,3,4-tetrahydroisoquinoline

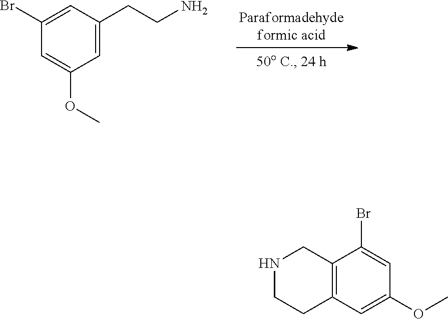

To a stirred solution of 2-(3-bromo-5-methoxyphenyl)ethan-1-amine (step 1c) (60 g, 262.0 mmol, 1.0 eq.) in formic acid (420 mL) was added paraformaldehyde (8.78 g) at room temperature. The reaction mixture was heated to 50° C. and stirred for 16 h. The resulting mixture was poured into ice and the pH adjusted to ~7-8 with saturated NaOH solution. The mixture was extracted with DCM (3×800 mL) and the combined organic extracts were dried over Na₂SO₄, filtered, and concentrated under vacuum. The crude material was purified by chromatography on silica (product eluted, 5% MeOH in DCM) yielding 8-bromo-6-methoxy-1,2,3,4-tetrahydroisoquinoline (77 g, Yield: 55%).

¹H NMR (DMSO, 400 MHz): δ 2.74-2.51 (m, 2H), 2.98-3.03 (m, 2H), 3.73-3.77 (m, 3H), 3.80-3.81 (m, 2H), 6.77 (d, J=2.4 Hz, 1H), 7.05 (m, 1H), 8.28 (s, 1H).

LCMS (Method A): 0.93 min, MS: ES+ 243.7 (M+1).

### Step 1: tert-Butyl 8-bromo-6-methoxy-3,4-dihydroisoquinoline-2(1H)-carboxylate

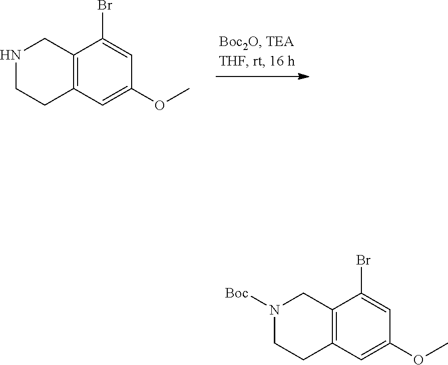 [SMILES: COc1cc(Br)c2c(c1)CCNC2 ; COc1cc(Br)c2c(c1)CCN(C(=O)OC(C)(C)C)C2]

The title compound was prepared from 8-bromo-6-methoxy-1,2,3,4-tetrahydroisoquinoline (step 1d), Boc anhydride and TEA analogously to Intermediate N step 1.

¹H NMR (DMSO-d6, 400 MHz): δ 1.43 (s, 9H), 1.47 (s, 2H), 2.76 (t, J=5.6 Hz, 2H), 3.75 (s, 3H), 4.37 (bs, 2H), 6.81 (s, 1H), 7.09 (s, 1H).

LCMS (Method A): 2.498 min, MS: ES+ 286 (M−56).

### Step 2: tert-butyl (S)-6-methoxy-8-((tetrahydrofuran-3-yl)amino)-3,4-dihydroisoquinoline-2(1H)-carboxylate

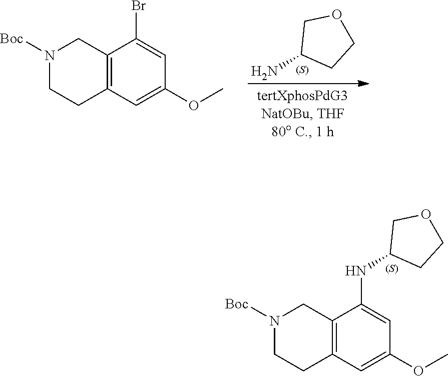

The title compound was prepared from tert-butyl 8-bromo-6-methoxy-3,4-dihydroisoquinoline-2(1H)-carboxylate (step 1) and (S)-tetrahydrofuran-3-amine analogously to Intermediate B1-1 step 1.

¹H NMR (DMSO-d6, 400 MHz): δ 1.25 (bs, 1H), 1.43 (s, 9H), 1.85-1.86 (m, 1H), 2.15-2.20 (m, 1H), 2.65 (t, J=5.6 Hz, 2H), 3.48 (bs, 2H), 3.57-3.82 (m, 5H), 3.84-3.99 (m, 5H), 4.20 (s, 2H), 4.95 (bs, 1H), 5.99-6.05 (m, 2H).

LCMS (Method A): 2.077 min, MS: ES+ 349 (M+1), MS: ES+ 292.9 (M−56).

### Step 3: (S)-6-Methoxy-N-(tetrahydrofuran-3-yl)-1,2,3,4-tetrahydroisoquinolin-8-amine hydrochloride

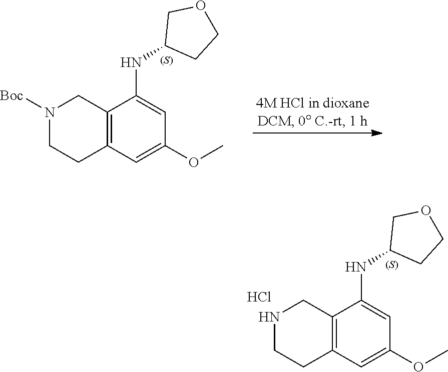

The title compound was prepared from tert-butyl (S)-6-methoxy-8-((tetrahydrofuran-3-yl)amino)-3,4-dihydroisoquinoline-2(1H)-carboxylate (step 2) and 4M HCl in dioxane analogously to Intermediate YM step 2.

¹H NMR (DMSO-d6, 400 MHz): δ 1.84-1.88 (m, 1H), 2.14-2.21 (m, 1H), 2.89 (t, J=5.7 Hz, 2H), 3.27-3.28 (m, 2H), 3.57 (s, 8H), 3.40-3.75 (m, 4H), 3.80-3.89 (m, 1H), 3.91 (t, J=2 Hz, 3H), 3.93-4.01 (m, 1H), 4.44 (bs, 4H), 6.04 (dd, J=2.4 Hz, 2H), 9.43 (bs, 2H).

LCMS (Method F): 1.66 min, MS: ES+ 249.3 (M+1).

### Intermediate ZI: (S)-6-((Dimethylamino)methyl)-N-(tetrahydrofuran-3-yl)-1,2,3,4-tetrahydroisoquinolin-8-amine hydrochloride

### Step 1: 8-Bromo-6-methoxy-1,2,3,4-tetrahydroisoquinoline hydrochloride

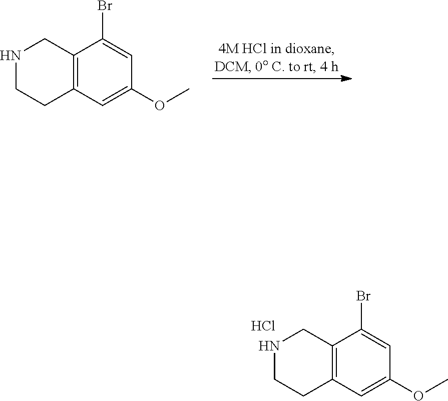

To a stirred solution 8-bromo-6-methoxy-1,2,3,4-tetrahydroisoquinoline (Intermediate ZH step 1d) (70 g, 289.2 mmol, 1.0 eq.) in DCM (700 mL) was added dropwise 4M HCl in dioxane (200 mL) at 0° C. and the reaction mixture was stirred at room temperature for 4 h. The resulting mixture was filtered and concentrated under vacuum. The crude material was triturated with n-pentane (2×100 mL) followed by high vacuum drying yielding 8-bromo-6-methoxy-1,2,3,4-tetrahydroisoquinoline hydrochloride (76 g, 313.89 mmol).

¹H NMR (DMSO, 400 MHz): δ 2.96-3.02 (m, 2H), 3.25-3.35 (m, 2H), 3.79 (m, 3H), 4.07 (s, 2H), 6.89 (d, J=2.4 Hz, 1H), 7.17 (d, J=2.4 Hz, 1H), 9.69 (s, 1H).

LCMS (Method A): 0.88 min, MS: ES+ 243.7 (M+1).

### Step 2: 8-Bromo-1,2,3,4-tetrahydroisoquinolin-6-ol

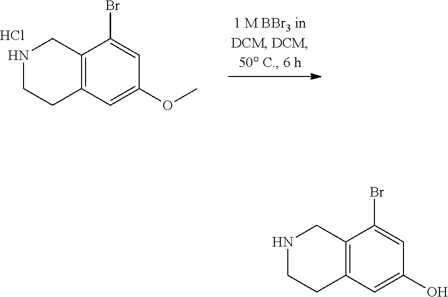

To a stirred solution 8-bromo-6-methoxy-1,2,3,4-tetrahydroisoquinoline hydrochloride (step 1) (33 g, 136.2 mmol, 1.0 eq.) in DCM (330 mL) was added dropwise BBr₃ (1M in DCM) (681.4 mL, 681.4 mmol) at 0° C. The reaction mixture was heated to 50° C. for 6 h. The resulting mixture was allowed to cool to room temperature, poured in ice cold water (3 L) and extracted with DCM (2×1000 mL). The aqueous layer was basified with aqueous NaOH (pH-7-8) causing precipitation of a white solid. The solid was collected by filtration, dried under vacuum, and triturated with n-pentane (2×200 mL) to yield 8-bromo-1,2,3,4-tetrahydroisoquinolin-6-ol as a white solid (45 g, Yield: 79%).

¹H NMR (DMSO, 400 MHz): δ 2.59 (t, J=5.6 Hz, J=11.2 Hz, 2H), 2.83 (t, J=6 Hz, J=11.6 Hz, 2H), 3.63 (s, 3H), 6.51 (d, J=1.6 Hz, 1H), 6.80 (d, J=2 Hz, 1H), 9.54 (s, 1H).

LCMS (Method A): 0.65 min, MS: ES+ 227.9 (M+1).

### Step 3: tert-Butyl 8-bromo-6-((tert-butoxycarbonyl)oxy)-3,4-dihydroisoquinoline-2(1H)-carboxylate

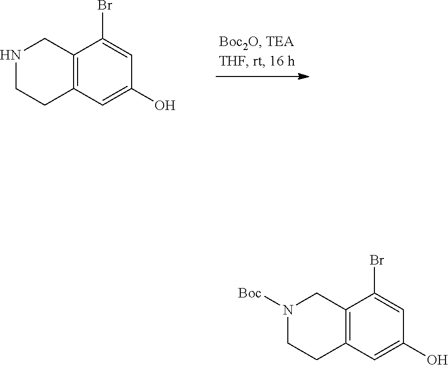 [SMILES: Oc1cc(Br)c2c(c1)CCNC2 ; CC(C)(C)OC(=O)N1CCc2cc(O)cc(Br)c2C1]

The title compound was prepared from 8-bromo-1,2,3,4-tetrahydroisoquinolin-6-ol (step 2), boc anhydride and TEA analogously to Intermediate N step 1.

¹H NMR (DMSO, 400 MHz): δ 1.43 (s, 9H), 1.50 (s, 9H), 2.80 (t, J=6 Hz, J=11.6 Hz, 2H), 3.54 (t, J=5.6 Hz, J=10.8 Hz, 2H), 4.43 (s, 2H), 7.11 (d, J=2.4 Hz, 1H), 7.44 (d, J=2.4 Hz, 1H).

LCMS (Method A): 2.76 min, MS: ES+ 273 (M+1).

### Step 4: tert-Butyl 8-bromo-6-(((trifluoromethyl) sulfonyl) oxy)-3,4-dihydroisoquinoline-2(1H)-carboxylate

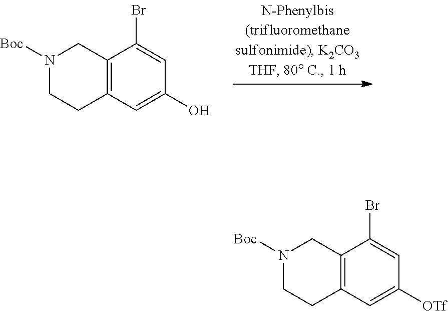 [SMILES: CC(C)(C)OC(=O)N1CCc2cc(O)cc(Br)c2C1 ; CC(C)(C)OC(=O)N1CCc2cc(OS(=O)(=O)C(F)(F)F)cc(Br)c2C1]

Performed in 2 parallel batches, each of 10 g scale: To a stirred solution of tert-butyl 8-bromo-6-hydroxy-3,4-dihydroisoquinoline-2(1H)-carboxylate (step 3) (10 g, 30.46 mmol, 1.0 eq.) in THE was added K₂CO₃ (8.41 g, 60.92 mmol, 2 eq.) and the mixture was stirred for 10 mins. N-Phenylbis(trifluoromethanesulfonimide) (CAS: 37595-74-7) (13.06 g, 36.56 mmol, 1.2 eq.) was added and the reaction mixture was heated to 80° C. for 1 h. The resulting mixture was poured into water (200 mL) and extracted with EtOAc (2×200 mL). The combined organic extracts were dried over Na₂SO₄, filtered, and concentrated under vacuum. The crude material was purified by chromatography on silica (product was eluted at 10% EtOAc in hexane) yielding tert-butyl 8-bromo-6-(((trifluoromethyl) sulfonyl) oxy)-3,4-dihydroisoquinoline-2(1H)-carboxylate (23 g, Yield: 82%).

¹H NMR (DMSO-d6, 400 MHz): δ 1.47 (s, 9H), 2.86 (t, J=5.6 Hz, 2H), 3.56 (t, J=5.2 Hz, 2H), 4.46 (s, 2H), 7.25-7.30 (m, 1H), 7.79 (d, J=2.4 Hz, 1H).

### Step 5: 2-(tert-Butyl) 6-methyl 8-bromo-3,4-dihydroisoquinoline-2,6(1H)-dicarboxylate

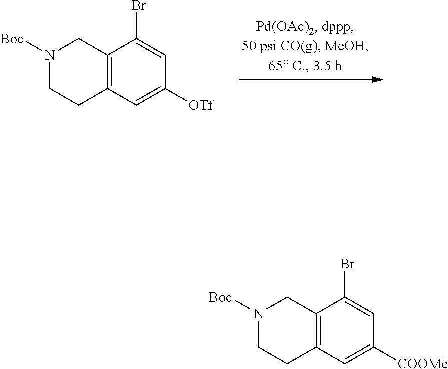 [SMILES: CC(C)(C)OC(=O)N1CCc2cc(OS(=O)(=O)C(F)(F)F)cc(Br)c2C1 ; COC(=O)c1cc(Br)c2c(c1)CCN(C(=O)OC(C)(C)C)C2]

The title compound was prepared from tert-butyl-8-bromo-6-(((trifluoromethyl)sulfonyl) oxy)-3,4-dihydroisoquinoline-2(1H)-carboxylate (step 4), DPPF and Pd(OAc)₂ analogously to Intermediate T step 2.

¹H NMR (DMSO-d6, 400 MHz): δ 1.43 (s, 9H), 2.88 (t, J=5.6 Hz, 2H), 3.57 (t, J=5.6, 2H), 3.86 (s, 3H), 4.50 (s, 2H), 7.80 (s, 1H), 7.97 (d, J=1.2 Hz, 1H).

LCMS (Method A): 2.44 min, MS: ES+ 315.6 (M−56).

### Step 6: tert-Butyl 8-bromo-6-(hydroxymethyl)-3,4-dihydroisoquinoline-2(1H)-carboxylate

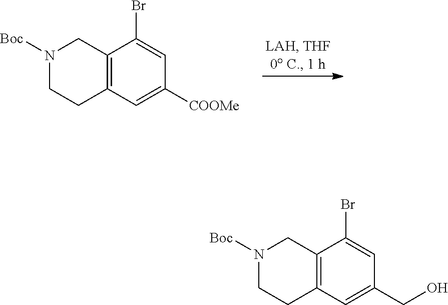 [SMILES: COC(=O)c1cc(Br)c2c(c1)CCN(C(=O)OC(C)(C)C)C2 ; CC(C)(C)OC(=O)N1CCc2cc(CO)cc(Br)c2C1]

To a cooled (0° C.) solution of 2-(tert-butyl) 6-methyl 8-bromo-3, 4-dihydroisoquinoline-2, 6(1H)-dicarboxylate (step 5) (4 g, 10.81 mmol, 1.0 eq) in THF was added dropwise lithium aluminum hydride solution (1.0 M in THF) (10.8 mL, 10.81 mmol, 1.0 eq) and the reaction mixture was stirred at 0° C. for 1 h. The reaction was quenched with NH₄Cl solution and filtered. The aqueous portion was extracted with EtOAc (2×100 mL) and the combined organic extracts were dried over Na₂SO₄, filtered and concentrated under vacuum yielding tert-butyl 8-bromo-6-(hydroxymethyl)-3,4-dihydroisoquinoline-2(1H)-carboxylate as a light brown sticky material (3.5 g, Yield: 90.2%).

¹H NMR (DMSO-d6, 400 MHz): δ 1.43 (s, 9H), 2.78 (t, J=5.6 Hz, 2H), 3.54 (t, J=5.6 Hz, 2H), 4.42-4.46 (m, 4H), 5.30 (t, J=6 Hz, 1H), 7.13 (s, 1H), 7.42 (s, 1H)

LCMS (Method A): 1.95 min, MS: ES+ 285.69 (M−56).

### Step 7: tert-Butyl 8-bromo-6-formyl-3, 4-dihydroisoquinoline-2(1H)-carboxylate

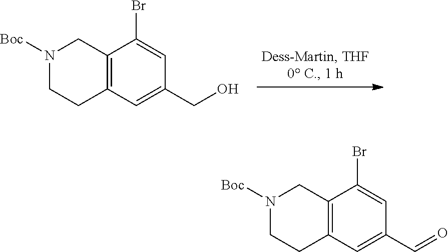 [SMILES: CC(C)(C)OC(=O)N1CCc2cc(CO)cc(Br)c2C1 ; CC(C)(C)OC(=O)N1CCc2cc(C=O)cc(Br)c2C1]

The title compound was prepared from tert-butyl 8-bromo-6-(hydroxymethyl)-3, 4-dihydroisoquinoline-2(1H)-carboxylate (step 6) and Dess martin periodinane analogously to Intermediate ZK step 3.

¹H NMR (DMSO-d6, 400 MHz): δ 1.42 (s, 9H), 2.90 (t, J=5.6 Hz, 2H), 3.59 (t, J=5.2 Hz, 2H), 4.52 (s, 2H), 7.70-7.75 (m, 1H), 7.98-8.00 (m, 1H), 9.94 (s, 1H).

### Step 8: tert-Butyl 8-bromo-6-((dimethylamino)methyl)-3,4-dihydroisoquinoline-2(1H)-carboxylate

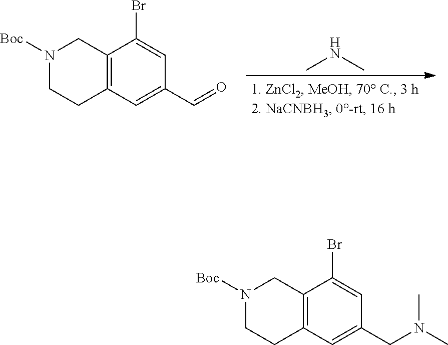

The title compound was prepared from tert-butyl 8-bromo-6-formyl-3,4-dihydroisoquinoline-2(1H)-carboxylate (step 7) and Dimethyl amine (2.0 M in THF) analogously to Intermediate YB step 3.

¹H NMR (DMSO-d6, 400 MHz): 1.43 (s, 9H), 2.32 (s, 3H), 2.78 (t, J=5.6 Hz, 2H), 3.17 (s, 2H), 3.34 (s, 3H), 3.55 (t, J=5.6 Hz, 2H), 4.47 (s, 2H), 7.19 (s, 1H), 7.48 (s, 1H)

LCMS (Method A): 1.410 min, MS: ES+ 370.9 (M+1).

### Step 9: tert-Butyl (S)-6-((dimethylamino)methyl)-8-((tetrahydrofuran-3-yl) amino)-3,4-dihydroisoquinoline-2(1H)-carboxylate

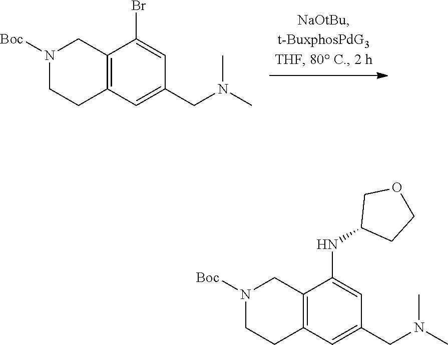 [SMILES: CN(C)Cc1cc(Br)c2c(c1)CCN(C(=O)OC(C)(C)C)C2 ; CN(C)Cc1cc2c(c(N[C@H]3CCOC3)c1)CN(C(=O)OC(C)(C)C)CC2]

The title compound was prepared from tert-butyl 8-bromo-6-((dimethylamino)methyl)-3,4-dihydroisoquinoline-2(1H)-carboxylate (step 1) and (S)-tetrahydrofuran-3-amine analogously to Intermediate B1-1 step 1.

LCMS (Method A): 1.306 min, MS: ES+ 376.1 (M+1).

### Step 10: (S)-6-((Dimethylamino)methyl)-N-(tetrahydrofuran-3-yl)-1,2,3,4-tetrahydroisoquinolin-8-amine hydrochloride

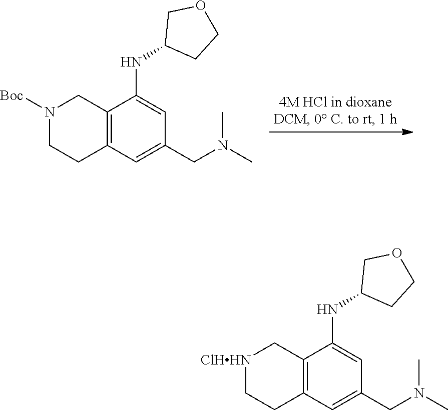

The title compound was prepared from tert-butyl (S)-6-((dimethylamino)methyl)-8-((tetrahydrofuran-3-yl) amino)-3,4-dihydroisoquinoline-2(1H)-carboxylate (step 9) and 4M HCl in dioxane analogously to Intermediate YM step 2.

LCMS (Method B): 1.58 min, MS: ES+ 276.3 (M+1).

### Intermediate ZJ: (S)-6-((1H-Imidazol-1-yl)methyl)-N-(tetrahydrofuran-3-yl)-1,2,3,4-tetrahydroisoquinolin-8-amine hydrochloride

### Step 1: tert-Butyl 8-bromo-6-(bromomethyl)-3,4-dihydroisoquinoline-2(1H)-carboxylate

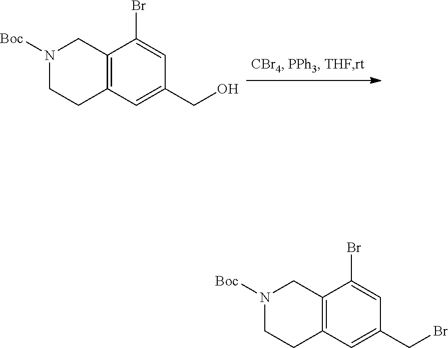 [SMILES: CC(C)(C)OC(=O)N1CCc2cc(CO)cc(Br)c2C1 ; CC(C)(C)OC(=O)N1CCc2cc(CBr)cc(Br)c2C1]

The title compound was prepared from tert-butyl 8-bromo-6-(hydroxymethyl)-3,4-dihydroisoquinoline-2(1H)-carboxylate (Intermediate ZI step 6), CBr₄ and triphenyl phosphine analogously to Intermediate R step 3.

¹H NMR (DMSO-d6, 400 MHz): δ 1.43 (s, 9H), 2.78 (t, J=5.6H, 2H), 3.55 (t, J=5.6H, 2H), 4.43 (s, 2H), 4.73 (s, 2H), 7.26 (s, 1H), 7.65 (s, 1H).

LCMS (Method A): 2.598 min, MS: ES+ 349.6 (M−56).

### Step 2: tert-Butyl 6-((1H-imidazol-1-yl)methyl)-8-bromo-3,4-dihydroisoquinoline-2(1H)-carboxylate

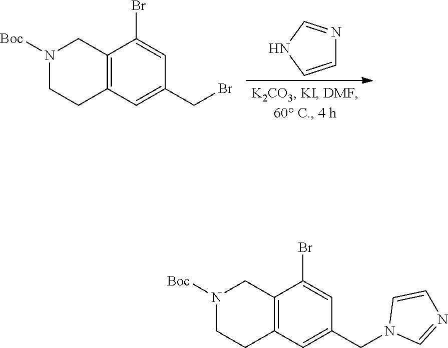 [SMILES: CC(C)(C)OC(=O)N1CCc2cc(CBr)cc(Br)c2C1 ; c1c[nH]cn1 ; CC(C)(C)OC(=O)N1CCc2cc(Cn3ccnc3)cc(Br)c2C1]

The title compound was prepared from tert-butyl 8-bromo-6-(bromomethyl)-3,4-dihydroisoquinoline-2(1H)-carboxylate (step 1) and 1H-imidazole analogously to Intermediate YF step 3.

¹H NMR (DMSO-d6, 400 MHz): δ 1.42 (s, 9H), 2.73-2.77 (m, 3H), 2.89 (s, 1H), 3.53 (t, J=5.6 Hz, 2H), 5.13 (s, 2H), 6.90 (s, 1H), 7.11 (s, 1H), 7.20 (s, 1H), 7.41 (s, 1H), 7.76 (s, 1H).

LCMS (Method A): 1.455 min, MS: ES+ 393.9 (M+1).

### Step 3: tert-Butyl (S)-6-((1H-imidazol-1-yl)methyl)-8-((tetrahydrofuran-3-yl)amino)-3,4-dihydroisoquinoline-2(1H)-carboxylate

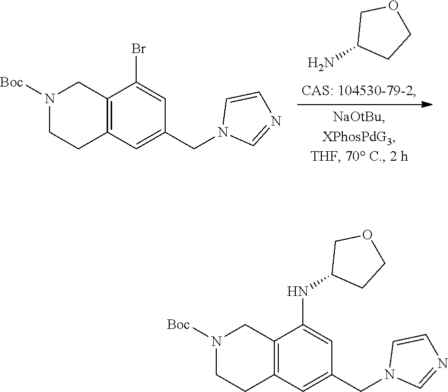 [SMILES: CC(C)(C)OC(=O)N1CCc2cc(Cn3ccnc3)cc(Br)c2C1 ; N[C@H]1CCOC1 ; CC(C)(C)OC(=O)N1CCc2cc(Cn3ccnc3)cc(N[C@H]3CCOC3)c2C1]

The title compound was prepared from tert-butyl 6-((1H-imidazol-1-yl)methyl)-8-bromo-3,4-dihydroisoquinoline-2(1H)-carboxylate (step 2) and (S)-tetrahydrofuran-3-amine analogously to Intermediate B1-1 step 1.

¹H NMR (DMSO-d6, 400 MHz): δ 1.42 (s, 9H), 1.80-1.83 (m, 1H), 2.14-2.19 (m, 1H), 2.65 (t, J=10H, 2H), 3.48 (t, J=5.2H, 2H), 3.52-3.55 (m, 1H), 3.71-3.79 (m, 1H), 3.81-3.89 (m, 1H), 3.91-3.96 (m, 2H), 4.24 (s, 1H), 5.04 (s, 2H), 6.35-6.37 (m, 2H), 6.88 (s, 1H), 7.16 (s, 1H), 7.72 (s, 1H), 9.90 (s, 2H).

LCMS (Method A): 1.337 min, MS: ES+ 399.2 (M+1).

### Step 4: (S)-6-((1H-imidazol-1-yl)methyl)-N-(tetrahydrofuran-3-yl)-1,2,3,4-tetrahydroisoquinolin-8-amine hydrochloride

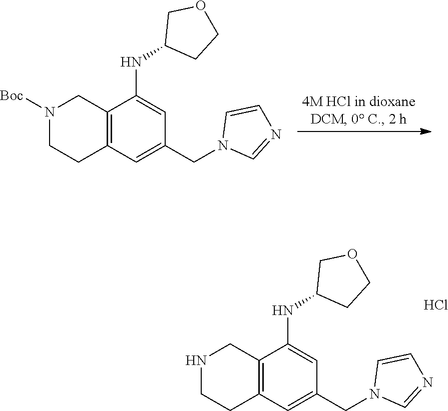 [SMILES: CC(C)(C)OC(=O)N1CCc2cc(Cn3ccnc3)cc(N[C@H]3CCOC3)c2C1 ; c1cn(Cc2cc3c(c(N[C@H]4CCOC4)c2)CNCC3)cn1 ; Cl]

The title compound was prepared from tert-butyl (S)-6-((1H-imidazol-1-yl)methyl)-8-((tetrahydrofuran-3-yl)amino)-3,4-dihydroisoquinoline-2(1H)-carboxylate (step 3) and 4M HCl in dioxane analogously to Intermediate YM step 2.

¹H NMR (DMSO-d6, 400 MHz): δ 1.84-1.86 (m, 1H), 2.19-2.24 (m, 1H), 2.14-2.19 (m, 1H), 2.90 (t, J=5.6 Hz, 2H), 3.22 (d, J=4 Hz, 2H), 3.53-3.57 (m, 1H), 3.71-3.76 (m, 1H), 3.81-3.86 (m, 1H), 3.94-3.98 (m, 3H), 4.00-4.03 (m, 1H), 5.31 (s, 2H), 6.36 (d, J=9.6 Hz, 2H), 6.87 (s, 1H), 7.16 (s, 1H), 7.72 (s, 1H).

LCMS (Method A): 1.77 min, MS: ES+ 299.3 (M+1).

### Intermediate ZK: (S)-7-Methoxy-N-(tetrahydrofuran-3-yl)-1,2,3,4-tetrahydroisoquinolin-8-amine hydrochloride

### Step 1: 8-7-methoxyisoquinoline

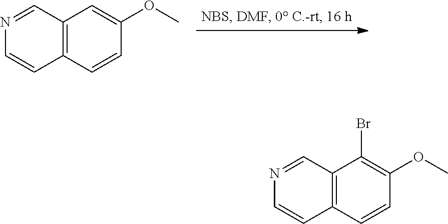

Performed in 4 parallel batches, each of 1 g scale: To a cooled (0° C.) solution of 7-methoxyisoquinoline (CAS 39989-34-4) (1 g, 6.28 mmol, 1 eq.) in DMF (10 mL) was added portion wise NBS (1.12 g, 6.28 mmol, 1.0 eq.) and the reaction mixture was stirred at room temperature for 16 h. The resulting mixture was diluted with water (50 mL) and extracted with EtOAc (3×50 mL). The combined organic extracts were dried over Na₂SO₄, filtered and concentrated under vacuum. The crude material was purified by chromatography on silica (eluting with 32% EtOAc in hexane) yielding 8-bromo-7-methoxyisoquinoline as an off-white solid (1.2 g, Yield: 20.04%)

¹H NMR (DMSO-d6, 400 MHz): δ 4.08 (s, 3H), 7.50 (d, J=8.8 Hz, 1H), 7.60 (dd, J=0.4 Hz, 1H), 7.84 (d, J=8.8 Hz, 1H), 8.51 (d, J=6 Hz, 1H), 9.64 (s, 1H).

LCMS (Method A): 0.911 min, MS: ES+ 239.6 (M+1)

### Step 2: 8-Bromo-7-methoxy-1,2,3,4-tetrahydroisoquinoline

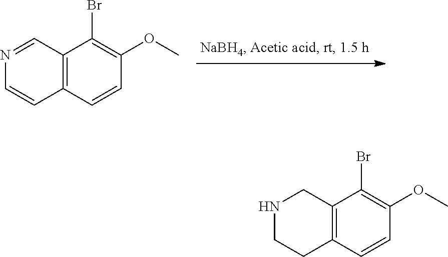

To a stirred solution of 8-bromo-7-methoxyisoquinoline (step 1)(1.1 g, 4.62 mmol, 1 eq.) in acetic acid (25.3 mL) was added portion wise NaBH₄ (0.61 g, 16.17 mmol, 3.5 eq.) and the mixture was stirred at room temperature for 1.5 h. The resulting mixture was diluted with water (30 mL), basified with saturated NaHCO₃ solution, and extracted with DCM (3×50 mL). The combined organic extracts were dried over Na₂SO₄, filtered, and concentrated under vacuum. The crude material was purified by trituration with n-pentane (3×20 mL) followed by high vacuum drying to yield 8-bromo-7-methoxy-1,2,3,4-tetrahydroisoquinoline as a yellow solid (1.5 g, Yield: Quantitative, 6.19 mmol).

¹H NMR (DMSO-d6, 400 MHz): δ 2.55 (d, J=7.2 Hz, 2H), 2.90 (t, J=5.2 Hz, 2H), 3.17 (s, 1H), 3.79 (d, J=8 Hz, 5H), 6.91 (dd, J=2.8 Hz, 1H), 7.10 (t, J=8.8 Hz, 1H).

LCMS (Method A): 0.781 min, MS: ES+ 243.7 (M+1)

### Step 3: tert-Butyl 8-bromo-7-methoxy-3,4-dihydroisoquinoline-2(1H)-carboxylate

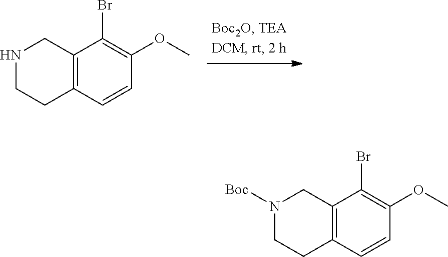 [SMILES: COc1ccc2c(c1Br)CNCC2 ; COc1ccc2c(c1Br)CN(C(=O)OC(C)(C)C)CC2]

The title compound was prepared from 8-bromo-7-methoxy-1,2,3,4-tetrahydroisoquinoline (step 2), boc anhydride and DIPEA analogously to Intermediate N step 1.

¹H NMR (DMSO-d6, 400 MHz): δ 1.43 (s, 9H), 2.73 (t, J=5.6 Hz, 2H), 3.53 (t, J=5.6 Hz, 2H), 3.83 (s, 3H), 4.44 (s, 2H), 6.98 (d, J=8.4 Hz, 1H), 7.17 (d, J=8.8 Hz, 1H).

LCMS (Method A): 2.358 min, MS: ES+ 287.6 (M−56).

### Step 4: tert-Butyl (S)-7-methoxy-8-((tetrahydrofuran-3-yl)amino)-3,4-dihydroisoquinoline-2(1H)-carboxylate

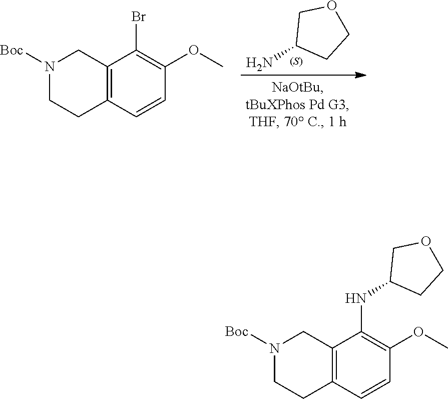

The title compound was prepared from tert-butyl 8-bromo-7-methoxy-3,4-dihydroisoquinoline-2(1H)-carboxylate (step 3) and (S)-tetrahydrofuran-3-amine analogously to Intermediate B1-1 step 1.

¹H NMR (DMSO-d6, 400 MHz): δ 1.48 (s, 9H), 1.72 (s, 1H), 1.94-1.99 (m, 1H), 2.70 (t, J=6 Hz, 2H), 3.47-3.89 (m, 9H), 4.36-4.44 (m, 2H), 6.48 (dd, J=6 Hz, 1H), 6.72 (d, J=8.4 Hz, 1H), 6.82 (d, J=8.4 Hz, 1H).

LCMS (Method A): 1.932 min, MS: ES+ 348.8 (M+1).

### Step 5: (S)-7-Methoxy-N-(tetrahydrofuran-3-yl)-1,2,3,4-tetrahydroisoquinolin-8-amine hydrochloride

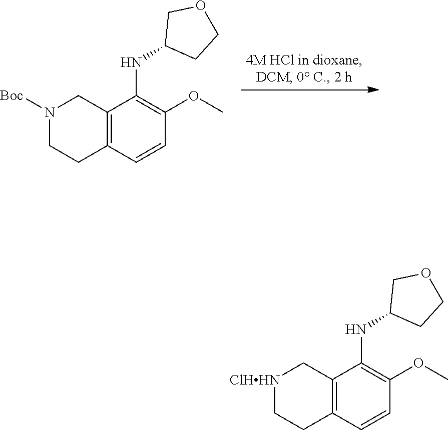

The title compound was prepared from tert-butyl (S)-7-methoxy-8-((tetrahydrofuran-3-yl)amino)-3,4-dihydroisoquinoline-2(1H)-carboxylate (step 4) and 4M HCl in dioxane analogously to Intermediate YM step 2.

¹H NMR (DMSO-d6, 400 MHz): δ 1.86 (t, J=4 Hz, 1H), 2.00-2.05 (m, 1H), 2.95 (t, J=6 Hz, 2H), 3.31 (d, J=4 Hz, 2H), 3.57-4.25 (m, 11H), 7.03 (dd, J=8.4 Hz, 2H), 9.52 (s, 1H).

LCMS (Method A): 0.805 min, MS: ES+ 249 (M+1).

### Intermediate ZL: (S)-6-(1-Methyl-1H-imidazol-5-yl)-N-(tetrahydrofuran-3-yl)isoindolin-4-amine hydrochloride

### Step 1: tert-Butyl (S)-6-hydroxy-4-((tetrahydrofuran-3-yl)amino)isoindoline-2-carboxylate

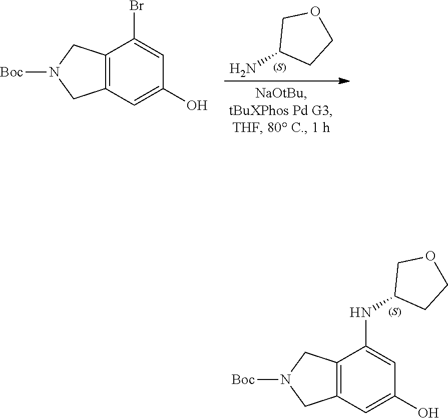

The title compound was prepared from tert-butyl 4-bromo-6-hydroxyisoindoline-2-carboxylate (Intermediate H step 2) and (S)-tetrahydrofuran-3-amine analogously to Intermediate B1-1 step 1.

¹H NMR (DMSO-d6, 400 MHz): δ 1.44-1.45 (m, 9H), 1.83-1.84 (m, 1H), 2.09-2.17 (m, 1H), 3.57 (t, J=4.4 Hz, 1H), 3.68-3.90 (m, 4H), 4.29-4.38 (m, 4H), 5.27-5.31 (m, 1H), 5.86 (s, 1H), 5.96 (d, J=5.2 Hz, 1H), 9.04 (d, J=3.6 Hz, 1H).

LCMS (Method A): 1.601 min, MS: ES+ 265.0 (M−56).

### Step 2: tert-Butyl (S)-4-((tetrahydrofuran-3-yl)amino)-6-(((trifluoromethyl)sulfonyl) oxy)isoindoline-2-carboxylate

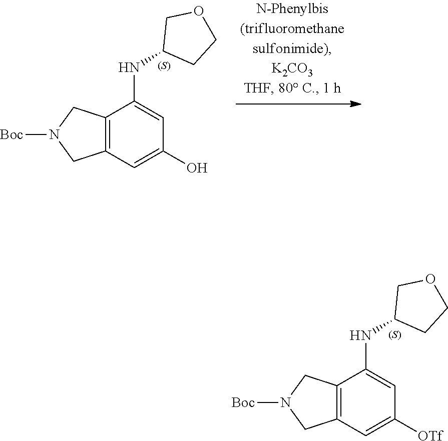

The title compound was prepared from tert-butyl (S)-6-hydroxy-4-((tetrahydrofuran-3-yl)amino)isoindoline-2-carboxylate (step 1) and N-phenylbis(trifluoromethanesulfonimide) analogously to Intermediate ZI step 4.

¹H NMR (DMSO-d6, 400 MHz): δ 1.40-1.47 (m, 9H), 1.81-1.83 (m, 1H), 2.20-2.33 (m, 1H), 3.57-3.62 (m, 1H), 3.69-4.06 (m, 4H), 4.41 (s, 2H), 4.51-4.55 (m, 2H), 5.95-6.02 (m, 1H), 6.46 (s, 1H), 6.63 (d, J=4.4 Hz, 1H).

LCMS (Method A): 2.393 min, MS: ES+ 396.9 (M−56).

### Step 3: tert-Butyl (S)-6-(1-methyl-1H-imidazol-5-yl)-4-((tetrahydrofuran-3-yl)amino)isoindoline-2-carboxylate

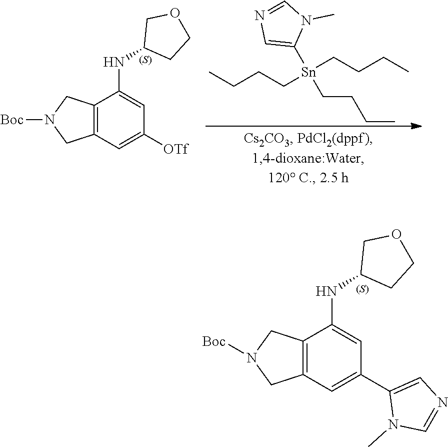

To a stirred solution of tert-butyl (S)-4-((tetrahydrofuran-3-yl)amino)-6-(((trifluoromethyl)sulfonyl)oxy)isoindoline-2-carboxylate (step 2) (0.3 g, 0.663 mmol, 1.0 eq) in 1,4-dioxane:water (3 mL) were added Cs₂CO₃ (0.648 g, 1.98 mmol, 3.0 eq) and 1-methyl-5-(tributylstannyl)-1H-imidazole (CAS: 147716-03-8) (0.369 g, 0.994 mmol, 1.5 eq) at room temperature. The reaction mixture was purged with nitrogen gas for 15 mins. PdCl₂(dppf) (0.048 g, 0.066 mmol, 0.1 eq) was added and the reaction mixture was heated to 120° C. for 2.5 h. The resulting mixture was concentrated under vacuum and purification of the crude material by chromatography on silica (3.9% MeOH in DCM) yielded tert-butyl (S)-6-(1-methyl-1H-imidazol-5-yl)-4-((tetrahydrofuran-3-yl)amino)isoindoline-2-carboxylate as yellow solid (0.2 g, 81.8%).

¹H NMR (DMSO-d6, 400 MHz): δ 1.46-1.51 (m, 9H), 1.85-1.86 (m, 1H), 2.15-2.20 (m, 1H), 3.59-3.66 (m, 4H), 3.69-3.74 (m, 1H), 3.82-4.11 (m, 2H), 4.44 (d, J=4.4 Hz, 1H), 4.53-4.56 (m, 1H), 5.53-5.57 (m, 1H), 6.46 (d, J=3.6 Hz, 1H), 6.66 (d, J=5.2 Hz, 1H), 6.97 (s, 1H), 7.66 (s, 1H).

LCMS (Method A): 1.285 min, MS: ES+ 385.0 (M+1).

### Step 4: (S)-6-(1-Methyl-1H-imidazol-5-yl)-N-(tetrahydrofuran-3-yl)isoindolin-4-amine hydrochloride

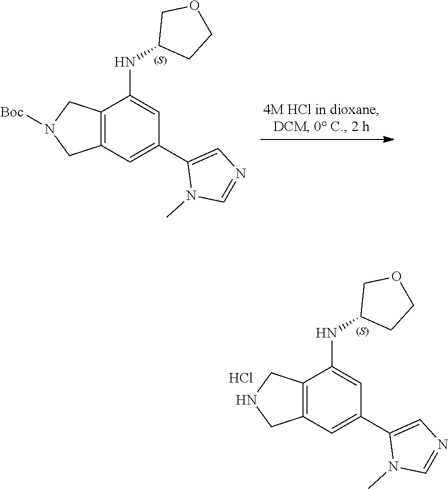

The title compound was prepared from tert-butyl (S)-6-(1-methyl-1H-imidazol-5-yl)-4-((tetrahydrofuran-3-yl)amino)isoindoline-2-carboxylate (step 3) and 4M HCl in dioxane analogously to Intermediate YD step 2.

¹H NMR (DMSO-d6, 400 MHz): δ 1.86-1.87 (m, 1H), 2.19-2.24 (m, 1H), 3.56-3.63 (m, 1H), 3.69-3.75 (m, 1H), 3.83 (s, 3H), 3.84-3.94 (m, 2H), 4.11-4.12 (m, 1H), 4.38-4.39 (m, 2H), 4.44-4.45 (m, 2H), 5.76 (s, 1H), 6.67 (s, 1H), 6.82 (s, 1H), 7.84 (s, 1H), 9.26 (s, 1H), 10.40 (s, 2H).

LCMS (Method B): 1.673 min, MS: ES+ 285.3 (M+1).

### Intermediate ZM: (S)-6-(1-Methyl-1H-imidazol-4-yl)-N-(tetrahydrofuran-3-yl)isoindolin-4-amine hydrochloride

### Step 1: tert-Butyl (S)-6-(1-methyl-1H-imidazol-4-yl)-4-((tetrahydrofuran-3-yl)amino)isoindoline-2-carboxylate

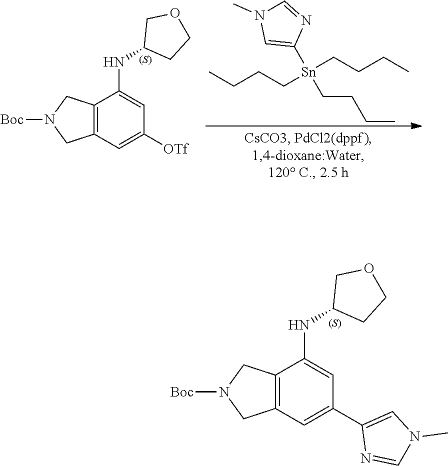

The title compound was prepared from tert-butyl (S)-4-((tetrahydrofuran-3-yl)amino)-6-(((trifluoromethyl)sulfonyl)oxy)isoindoline-2-carboxylate (Intermediate ZL step 2) and 1-methyl-4-(tributylstannyl)-1H-imidazole (CAS 446285-73-0) analogously to Intermediate ZL step 3.

¹H NMR (DMSO-d6, 400 MHz): δ 1.46-1.47 (m, 9H), 1.84-1.88 (m, 1H), 2.17-2.23 (m, 1H), 3.57-3.60 (m, 1H), 3.68 (s, 3H), 3.73-3.77 (m, 1H), 3.81-3.85 (m, 1H), 3.94 (t, J=8.4 Hz, 1H), 4.08-4.09 (m, 1H), 4.41 (d, J=5.2 Hz, 2H), 4.49-4.52 (m, 2H), 6.87 (d, J=5.2 Hz, 1H), 6.92-6.96 (m, 1H), 7.52-7.54 (m, 1H), 7.60 (s, 1H).

LCMS (Method A): 1.282 min, MS: ES+ 385.0 (M+1).

### Step 2: (S)-6-(1-Methyl-1H-imidazol-4-yl)-N-(tetrahydrofuran-3-yl)isoindolin-4-amine hydrochloride

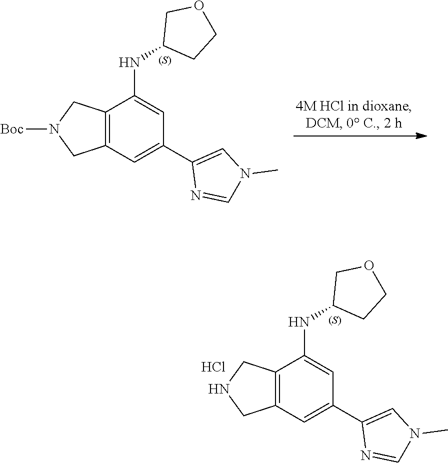

The title compound was prepared from tert-butyl (S)-6-(1-methyl-1H-imidazol-4-yl)-4-((tetrahydrofuran-3-yl)amino)isoindoline-2-carboxylate (step 1) and 4M HCl in dioxane analogously to Intermediate YD step 2.

¹H NMR D₂O exchange (DMSO-d6, 400 MHz): δ 1.84-1.88 (m, 1H), 2.32-2.33 (m, 1H), 3.57-3.60 (m, 1H), 3.62 (s, 3H), 3.72-3.78 (m, 1H), 3.84-3.89 (m, 1H), 3.98-4.02 (m, 1H), 4.14-4.17 (m, 2H), 4.36 (s, 2H), 4.47 (s, 2H), 6.95 (s, 1H), 7.02 (s, 1H), 8.16 (s, 1H), 9.18 (s, 1H).

LCMS (Method A): 1.719 min, MS: ES+ 285.3 (M+1).

### Intermediate ZN: (S)-6-(1-Methyl-1H-imidazol-5-yl)-N-(tetrahydrofuran-3-yl)-1,2,3,4-tetrahydroisoquinolin-8-amine hydrochloride

### Step 1: tert-Butyl (S)-6-hydroxy-8-((tetrahydrofuran-3-yl)amino)-3,4-dihydroisoquinoline-2(1H)-carboxylate

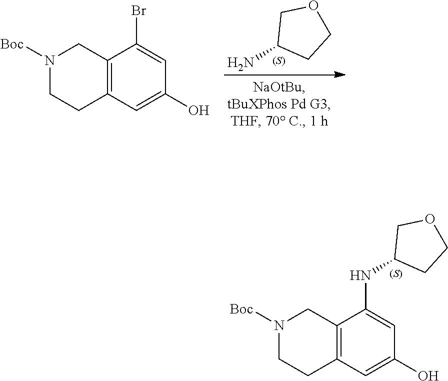

The title compound was prepared from tert-Butyl 8-bromo-6-((tert-butoxycarbonyl)oxy)-3,4-dihydroisoquinoline-2(1H)-carboxylate (Intermediate ZI step 3) and (S)-tetrahydrofuran-3-amine analogously to Intermediate B1-1 step 1.

¹H NMR (DMSO-d6, 400 MHz): δ 1.43 (s, 9H), 1.85-1.87 (m, 1H), 2.10-2.17 (m, 1H), 2.57 (t, J=4 Hz, J=12 Hz, 2H), 3.46 (s, 2H), 3.56-3.61 (m, 1H), 3.69-3.74 (m, 1H), 3.79-3.85 (m, 1H), 3.87-3.90 (m, 2H), 4.17 (s, 2H), 4.68-4.76 (bs, 1H) (s, 1H), 6.51 (s, 1H).

LCMS (Method A): 1.662 min, MS: ES+ 278.9 (M−56).

### Step 2: tert-Butyl (S)-8-((tetrahydrofuran-3-yl) amino)-6-(((trifluoromethyl) sulfonyl) oxy)-3,4-dihydroisoquinoline-2(1H)-carboxylate

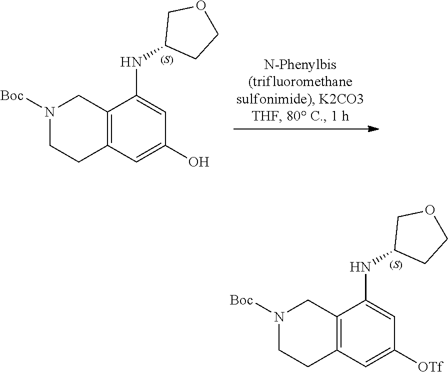

The title compound was prepared from tert-butyl (S)-6-hydroxy-8-((tetrahydrofuran-3-yl)amino)-3,4-dihydroisoquinoline-2(1H)-carboxylate (step 1) and N-phenylbis(trifluoromethanesulfonimide) analogously to Intermediate ZI step 4.

¹H NMR (DMSO-d6, 400 MHz): δ 1.43 (s, 9H), 1.82-1.90 (m, 1H), 2.15-2.23 (m, 1H), 2.71-2.73 (m, 2H), 3.52 (s, 2H), 3.61-3.64 (m, 1H), 3.70-3.76 (m, 1H), 3.81-3.87 (m, 1H), 3.89-3.93 (m, 1H), 4.04 (s, 1H), 4.28 (s, 2H) 6.47 (s, 1H), 6.51 (s, 1H).

LCMS (Method A): 2.41 min, MS: ES+ 410.9 & 336.9 (M−56 & M−100).

### Step 3: tert-Butyl (S)-6-(1-methyl-1H-imidazol-5-yl)-8-((tetrahydrofuran-3-yl) amino)-3,4-dihydroisoquinoline-2(1H)-carboxylate

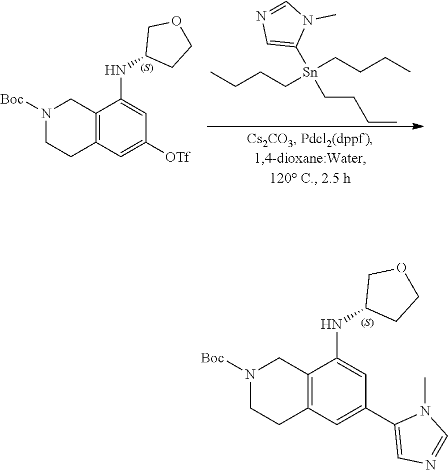

The title compound was prepared from tert-butyl (S)-8-((tetrahydrofuran-3-yl) amino)-6-(((trifluoromethyl) sulfonyl) oxy)-3,4-dihydroisoquinoline-2(1H)-carboxylate (step 2) and 1-methyl-5-(tributylstannyl)-1H-imidazole (CAS: 147716-03-8) analogously to Intermediate ZL step 3.

¹H NMR (DMSO-d6, 400 MHz): δ 0.87 (t, J=8 Hz, J=4 Hz, 1H), 1.11-1.25 (m, 1H), 1.28-1.34 (m, 1H), 1.44 (s, 9H), 1.60 (s, 1H), 1.88 (m, 1H), 2.17-2.22 (m, 1H), 2.73 (t, J=8 Hz, J=12 Hz, 2H), 3.54 (s, 2H), 3.62-3.66 (m, 1H), 3.70-3.75 (m, 1H), 3.82-3.85 (m, 1H), 3.92-3.95 (m, 1H), 4.10-4.12 (m, 1H), 4.31 (s, 2H), 5.0 (s, 1H), 6.49 (s, 1H), 6.56 (s, 1H), 6.96 (s, 1H), 7.65 (s, 1H).

LCMS (Method A): 1.354 min, MS: ES+ 399.1 & 400.2 (M+1 & M+2).

### Step 4: (S)-6-(1-Methyl-1H-imidazol-5-yl)-N-(tetrahydrofuran-3-yl)-1,2,3,4-tetrahydroisoquinolin-8-amine hydrochloride

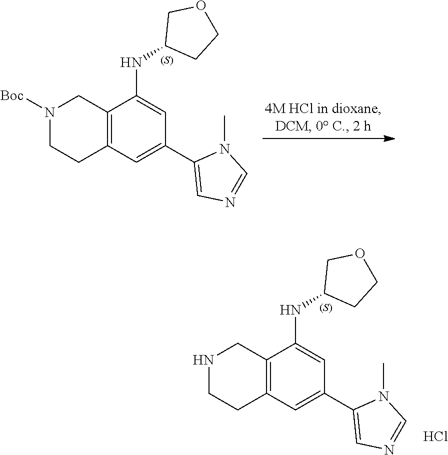

The title compound was prepared from tert-butyl (S)-6-(1-methyl-1H-imidazol-5-yl)-8-((tetrahydrofuran-3-yl) amino)-3,4-dihydroisoquinoline-2(1H)-carboxylate (step 3) and 4M HCl in dioxane analogously to Intermediate YD step 2.

¹H NMR (DMSO-d6, 400 MHz): δ 1.90-1.93 (m, 1H), 2.17-2.26 (m, 1H), 2.98-3.01 (m, 2H), 3.29 (s, 2H), 3.62 (dd, J=4 Hz, 1H), 3.70-03.74 (m, 1H), 3.87 (t, J=4 Hz, J=8 Hz, 4H), 3.93-3.97 (m, 1H), 4.03 (s, 2H), 4.08-4.11 (m, 1H), 6.66-6.72 (m, 2H), 7.83 (s, 1H), 9.25 (s, 1H), 10.05 (s, 2H).

LCMS (Method F): 1.53 min, MS: ES+ 299.4 (M+1).

### Intermediate ZO: (S)-6-(1-Methyl-1H-imidazol-4-yl)-N-(tetrahydrofuran-3-yl)-1,2,3,4-tetrahydroisoquinolin-8-amine hydrochloride

### Step 1: tert-Butyl (S)-6-(1-methyl-1H-imidazol-4-yl)-8-((tetrahydrofuran-3-yl) amino)-3,4-dihydroisoquinoline-2(1H)-carboxylate

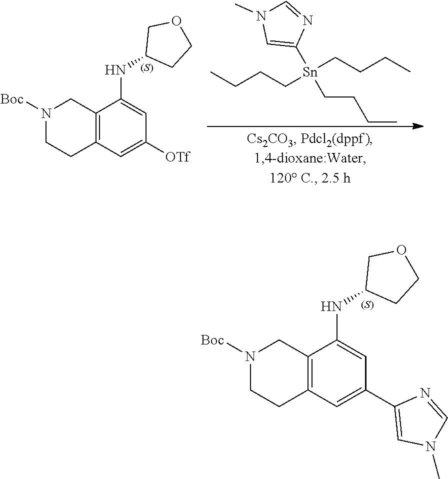

The title compound was prepared from tert-butyl (S)-8-((tetrahydrofuran-3-yl) amino)-6-(((trifluoromethyl) sulfonyl) oxy)-3,4-dihydroisoquinoline-2(1H)-carboxylate (Intermediate ZN step 2) and 1-methyl-4-(tributylstannyl)-1H-imidazole (CAS: 446285-73-0) analogously to Intermediate ZL step 3.

¹H NMR (DMSO-d6, 400 MHz): δ 1.24-1.30 (m, 1H), 1.44 (s, 9H), 1.88-1.89 (m, 1H), 2.20-2.25 (m, 1H), 2.70 (t, J=4 Hz, J=11.2 Hz, 2H), 3.52 (s, 2H), 3.59-3.62 (m, 1H), 3.66 (s, 3H), 3.72-3.78 (m, 1H), 3.83-3.86 (m, 1H), 3.95-3.98 (m, 1H), 4.07-4.09 (m, 1H), 4.28 (s, 2H), 6.87 (s, 1H), 7.53 (s, 1H), 7.57 (s, 1H).

LCMS (Method A): 1.361 min, MS: ES+ 399.1 & 400.2 (M+1 & M+2).

### Step 2: (S)-6-(1-Methyl-1H-imidazol-4-yl)-N-(tetrahydrofuran-3-yl)-1,2,3,4-tetrahydroisoquinolin-8-amine hydrochloride

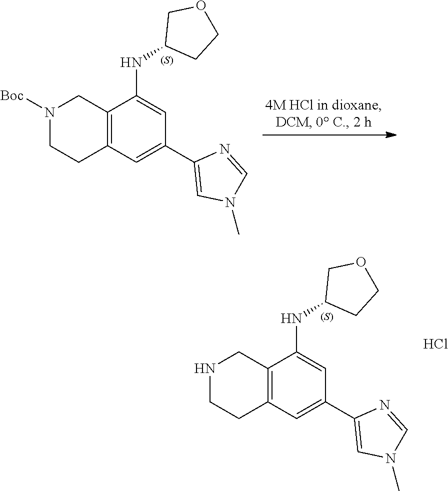

The title compound was prepared from tert-butyl (S)-6-(1-methyl-1H-imidazol-4-yl)-8-((tetrahydrofuran-3-yl) amino)-3,4-dihydroisoquinoline-2(1H)-carboxylate (step 1) and 4M HCl in dioxane analogously to Intermediate YD step 2.

LCMS (Method Z): 1.54 min, MS: ES+ 299.4 & M+1.

### Intermediate ZP: N-(2-Methoxyethyl)isoindolin-4-amine hydrochloride

### Step 1: tert-Butyl 4-((2-methoxyethyl)amino)isoindoline-2-carboxylate

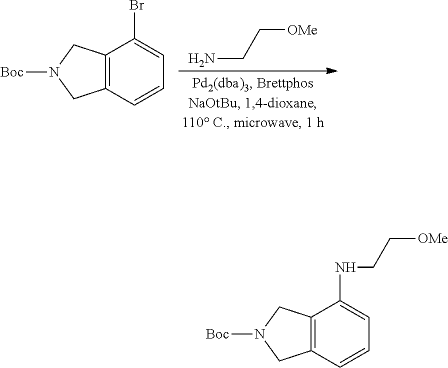

The title compound was prepared from tert-butyl 4-bromoisoindoline-2-carboxylate (Intermediate N step 1) and 2-methoxyethan-1-amine analogously to Intermediate B step 1.

¹H NMR (DMSO-d6, 400 MHz): δ 1.45 (s, 9H), 3.21-3.26 (m, 2H), 3.34 (s, 3H), 3.44-3.50 (m, 2H), 4.38 (d, J=8 Hz, 2H), 4.50 (d, J=11.2 Hz, 2H), 5.25-5.29 (m, 1H), 6.46 (d, J=8.4 Hz, 1H), 6.52 (t, J=6.8 Hz, 7.2 Hz, 1H), 7.07 (t, J=7.6 Hz, 1H).

LCMS (Method A): 2.082 min, MS: ES+292.9 (M+1).

### Step 2: N-(2-Methoxyethyl)isoindolin-4-amine hydrochloride

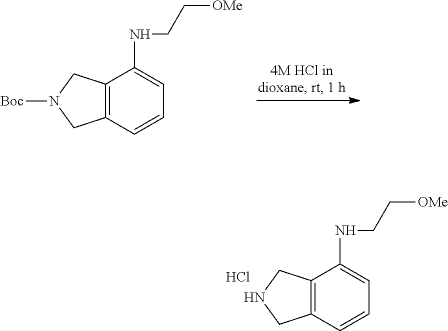 [SMILES: COCCNc1cccc2c1CN(C(=O)OC(C)(C)C)C2 ; Cl ; COCCNc1cccc2c1CNC2]

The title compound was prepared from tert-butyl 4-((2-methoxyethyl)amino)isoindoline-2-carboxylate (step 1) and 4M HCl in dioxane analogously to Intermediate B step 2.

¹H NMR (DMSO-d6, 400 MHz): δ 3.27 (t, J=6.4 Hz, 5H), 3.49 (t, J=5.6 Hz, 2H), 4.31 (t, J=5.2 Hz, 2H), 4.40 (t, J=5.6 Hz, 2H), 6.57-6.63 (m, 2H), 7.16 (t, J=7.6 Hz, 1H), 9.83 (s, 2H).

LCMS (Method A): 0.783 min, MS: ES+192.9 (M+1).

### Intermediate ZQ: 2-(Isoindolin-4-ylamino)-N, N-dimethylacetamide hydrochloride

### Step 1: tert-Butyl 4-((2-(dimethyl amino)-2-oxoethyl) amino) isoindoline-2-carboxylate

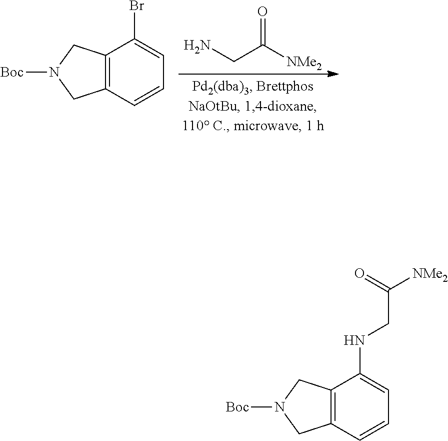

The title compound was prepared from tert-butyl 4-bromoisoindoline-2-carboxylate (Intermediate N step 1) and 2-amino-N, N-dimethylacetamide (CAS: 1857-19-8) analogously to Intermediate B step 1.

¹H NMR (DMSO-d6, 400 MHz): δ 1.38 (s, 9H), 3.02 (s, 3H), 2.86 (s, 3H), 3.92 (d, J=5.2 Hz, 2H), 4.66-4.54 (m, 4H), 5.13-5.19 (m, 1H), 6.45-6.42 (m, 1H), 6.57 (t, J=7.2 Hz, 1H), 7.08 (t, J=6.4 Hz, 1H).

LCMS (Method A): 1.802 min, MS: ES+219.93 (M−100).

### Step 2: 2-(Isoindolin-4-ylamino)-N, N-dimethylacetamide hydrochloride

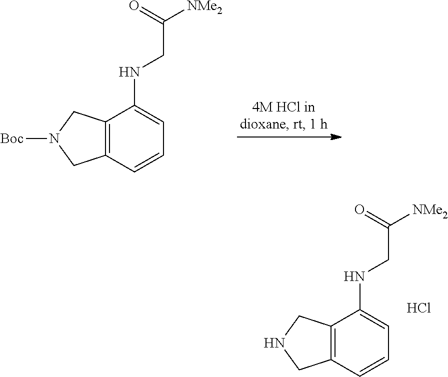 [SMILES: CN(C)C(=O)CNc1cccc2c1CN(C(=O)OC(C)(C)C)C2 ; CN(C)C(=O)CNc1cccc2c1CNC2 ; Cl]

The title compound was prepared from tert-butyl 5-(2-morpholinoethoxy) isoindoline-2-carboxylate (step 1) and 4M HCl in dioxane analogously to Intermediate B step 2.

¹H NMR (DMSO-d6, 400 MHz): δ 2.86 (s, 3H), 3.03 (s, 3H), 3.97 (s, 2H), 6.45-6.42 (m, 4H), 6.48 (d, J=7.6 Hz, 1H), 6.62 (d, J=7.2 Hz, 1H), 7.14 (t, J=8.0 Hz, 1H), 9.38 (s, 2H).

LCMS (Method F): 1.52 min, MS: ES+ 220.3 (M+1).

### Intermediate ZR: 2-(Isoindolin-5-yloxy)-N,N-dimethylethan-1-amine hydrochloride

### Step 1: 5-(2-(Dimethylamino)ethoxy)isoindoline-2-carboxylate

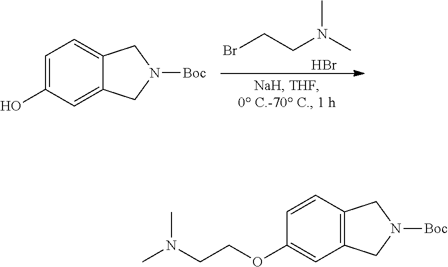

The title compound was prepared from tert-butyl 5-hydroxyisoindoline-2-carboxylate (Intermediate V step 2), 2-bromo-N,N-dimethylethan-1-amine hydrobromide (CAS: 2862-39-7) and NaH analogously to Intermediate M step 1.

1H NMR (DMSO-d6, 400 MHz): δ 1.45 (s, 9H), 2.23 (s, 6H), 2.64 (t, J=5.6 Hz, 2H), 4.01-4.04 (m, 2H), 4.51 (dd, J=10.8, 14 Hz, 4H), 6.85 (d, J=8.4 Hz, 1H), 6.91 (d, J=6.8 Hz, 1H), 7.20 (dd, J=5.6, 8 Hz, 1H).

### Step 2: 2-(Isoindolin-5-yloxy)-N,N-dimethylethan-1-amine hydrochloride

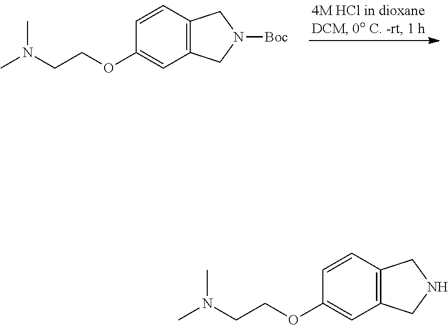 [SMILES: CN(C)CCOc1ccc2c(c1)CN(C(=O)OC(C)(C)C)C2 ; CN(C)CCOc1ccc2c(c1)CNC2]

The title compound was prepared from tert-butyl 5-(2-(dimethylamino)ethoxy)isoindoline-2-carboxylate and 4M HCl in dioxane analogously to Intermediate M step 3.

¹H NMR (DMSO-d6, 400 MHz): δ 2.81 (d, J=4.8 Hz, 6H), 3.47-3.51 (m, 2H), 4.32-4.46 (m, 6H), 7.00 (dd, J=2, 8.4 Hz, 1H), 7.06 (t, J=7.2 Hz, 1H), 7.33 (d, J=8.4 Hz, 1H), 10.27 (bs, 2H).

LCMS (Method N): 1.067 min, MS: ES+ 207.3 (M+1).

### Intermediate ZS: N-Methylisoindolin-4-amine hydrochloride

### Step 1: tert-Butyl 4-(methylamino)isoindoline-2-carboxylate

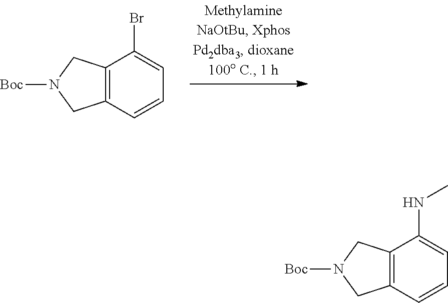 [SMILES: CC(C)(C)OC(=O)N1Cc2cccc(Br)c2C1 ; CNc1cccc2c1CN(C(=O)OC(C)(C)C)C2]

The title compound was prepared from tert-butyl 4-bromoisoindoline-2-carboxylate and methyl amine (2M in THF) analogously to Intermediate B1-1 step 1.

LCMS (Method A): 1.837 min, MS: ES+ 192.9 (M−56).

¹H NMR (DMSO-d6, 400 MHz): δ 1.46 (d, J=4 Hz, 9H), 2.68-2.70 (q, J=3.2 Hz, 4.4 Hz, 3H), 4.37 (d, J=10 Hz, 2H), 4.50 (d, J=10 Hz, 2H), 5.39-5.42 (q, J=4.8 Hz, 8.8 Hz, 1H), 6.37 (d, J=8 Hz, 1H), 6.51 (t, J=7.2 Hz, 1H), 7.09 (t, J=7.8 Hz, 1H).

### Step 2: N-Methylisoindolin-4-amine hydrochloride

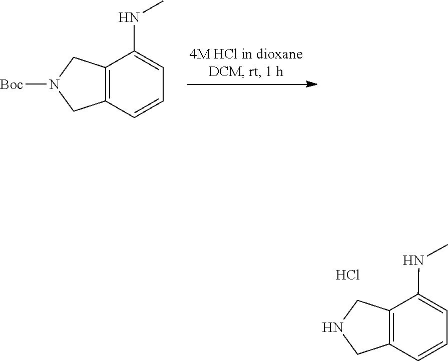 [SMILES: CNc1cccc2c1CN(C(=O)OC(C)(C)C)C2 ; CNc1cccc2c1CNC2 ; Cl]

The title compound was prepared from tert-butyl 4-(methylamino)isoindoline-2-carboxylate (step 1) and 4M HCl in dioxane analogously to Intermediate YM step 2.

LCMS (Method A): 0.858 min, MS: ES+148.87 (M+1).

¹H NMR (DMSO-d6, 400 MHz): δ 2.76 (s, 3H), 4.44 (t, J=5.4 Hz, 4H), 6.17 (bs, 3H), 6.76-6.85 (m, 2H), 7.27 (t, J=7.8 Hz, 1H), 10.09 (s, 2H).

### Intermediate ZT: N-Methyl-1,2,3,4-tetrahydroisoquinolin-8-amine hydrochloride

### Step 1: tert-Butyl 8-(methylamino)-3,4-dihydroisoquinoline-2(1H)-carboxylate

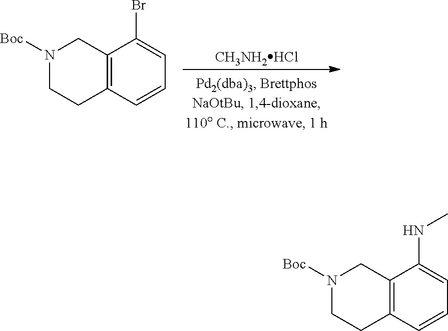

The title compound was prepared from tert-butyl 8-bromo-3,4-dihydroisoquinoline-2(1H)-carboxylate (CAS: 893566-75-1) and methylamine·HCl analogously to Intermediate A step 1

¹H NMR (DMSO-d6, 400 MHz): δ 1.42 (s, 9H), 2.65-2.69 (m, 5H), 3.48-3.51 (m, 2H), 4.21 (s, 2H), 5.07 (s, 1H), 6.35-6.40 (m, 2H), 6.99 (t, J=7.6 Hz, 1H)

LCMS (Method A): 2.109 min, MS: ES+207.13 (M−56).

### Step 2: N-Methyl-1,2,3,4-tetrahydroisoquinolin-8-amine hydrochloride

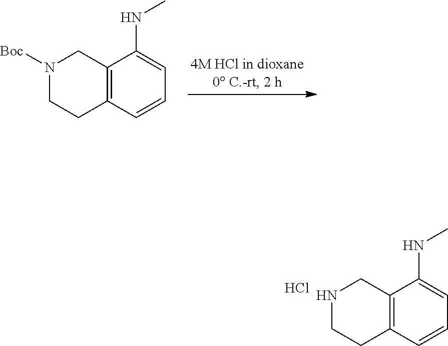 [SMILES: CNc1cccc2c1CN(C(=O)OC(C)(C)C)CC2 ; Cl ; CNc1cccc2c1CNCC2]

The title compound was prepared from tert-butyl 8-(methylamino)-3,4-dihydroisoquinoline-2(1H)-carboxylate (step 1) and 4M HCl in 1,4-dioxane analogously to Intermediate A step 2.

¹H NMR, D₂O exchange (DMSO-d6, 400 MHz): δ 2.74 (s, 3H), 2.93-2.96 (m, 2H), 3.28-3.31 (m, 2H), 4.05 (s, 2H), 6.68 (d, J=7.6 Hz, 2H), 7.18 (t, J=8 Hz, 1H)

LCMS (Method A): 0.767 min, MS: ES+163.17 (M+1), 254 nm

### Intermediate ZU: N-(2-Methoxyethyl)-1,2,3,4-tetrahydroisoquinolin-8-amine hydrochloride

### Step 1: tert-Butyl 8-((2-methoxyethyl)amino)-3,4-dihydroisoquinoline-2(1H)-carboxylate

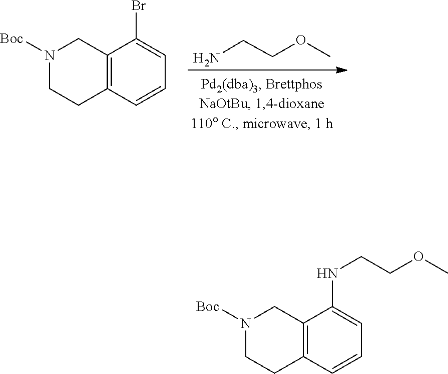

The title compound was prepared from tert-butyl 8-bromo-3,4-dihydroisoquinoline-2(1H)-carboxylate (CAS: 893566-75-1) and 2-methoxyethan-1-amine (CAS: 109-85-3) analogously to Intermediate B step 1.

¹H NMR (DMSO-d6, 400 MHz): δ 1.43 (s, 9H), 2.67 (t, J=1.6 Hz, 2H), 3.24 (q, J=6 Hz, 2H), 3.34 (s, 3H), 3.51 (m, 4H), 3.81 (s, 2H), 4.78 (s, 1H), 6.45 (m, 2H), 6.98 (t, J=8 Hz, 1H).

LCMS (Method A): 2.149 min, MS: ES+307.0 (M+1).

### Step 2: N-(2-Methoxyethyl)-1,2,3,4-tetrahydroisoquinolin-8-amine hydrochloride

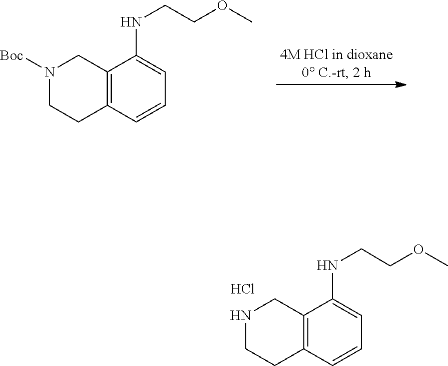 [SMILES: COCCNc1cccc2c1CN(C(=O)OC(C)(C)C)CC2 ; Cl ; COCCNc1cccc2c1CNCC2]

The title compound was prepared from tert-butyl 8-((2-methoxyethyl)amino)-3,4-dihydroisoquinoline-2(1H)-carboxylate (step 1) and 4M HCl in dioxane analogously to Intermediate B step 2.

¹H NMR (DMSO-d6, 400 MHz): δ 2.93 (t, J=5.6 Hz, 2H), 3.27 (m, 7H), 3.52 (t, J=5.6 Hz, 2H), 3.99 (s, 2H), 6.59 (m, 2H), 7.10 (t, J=7.6 Hz, 1H), 9.64 (s, 2H).

LCMS (Method A): 1.76 min, MS: ES+ 207.2 (M+1).

### Intermediate ZV: 4-((Tetrahydrofuran-3-yl)oxy)isoindoline hydrochloride

### Step 1a: tert-Butyl 4-(4,4,5,5-tetramethyl-1,3,2-dioxaborolan-2-yl)isoindolidine-2-carboxylate

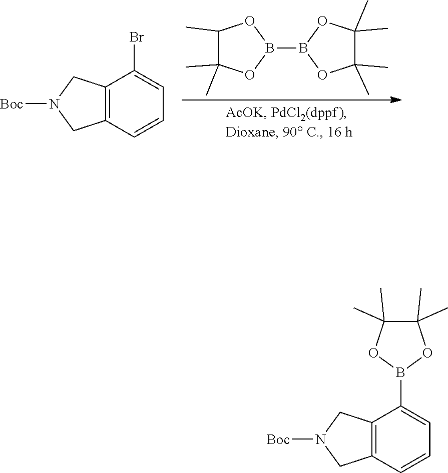

The title compound was prepared from tert-butyl 4-bromoisoindoline-2-carboxylate (Intermediate N step 1) and bis pinacolato diborane analogously to Intermediate V step 1.

¹H NMR (DMSO-d6, 400 MHz): δ 1.30 (s, 12H), 1.46 (s, 9H), 4.55-4.58 (m, 2H), 4.65-4.67 (m, 2H), 7.31-7.28 (m, 1H), 7.45 (d, J=7.2 Hz, 1H), 7.58 (d, J=6.4 Hz, 1H),

LCMS (Method A): 2.821 min, MS: ES+246.1 (M−100).

### Step 1 b: tert-Butyl 4-hydroxyisoindoline-2-carboxylate

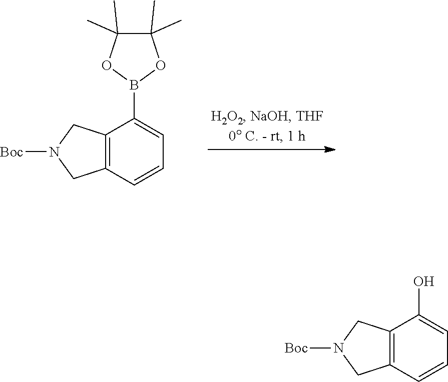 [SMILES: CC(C)(C)OC(=O)N1Cc2cccc(B3OC(C)(C)C(C)(C)O3)c2C1 ; CC(C)(C)OC(=O)N1Cc2cccc(O)c2C1]

The title compound was prepared from tert-butyl 4-(4,4,5,5-tetramethyl-1,3,2-dioxaborolan-2-yl)isoindoline-2-carboxylate (step 1a) and NaOH/H₂O₂ analogously to Intermediate V step 2.

¹H NMR (CDCl₃, 400 MHz): δ 1.54 (s, 9H), 4.65-4.70 (m, 3H), 4.81 (s, 1H), 6.78 (d, J=7.6 Hz, 1H), 7.14-7.19 (m, 1H), 6.78 (d, J=8 Hz, 1H), 7.19-7.14 (m, 1H).

LCMS (Method A): 1.999 min, MS: ES+136.1 (M−100)

### Step 1: tert-Butyl 4-((tetrahydrofuran-3-yl)oxy)isoindoline-2-carboxylate

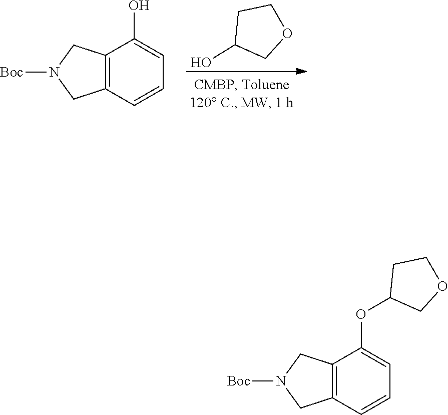

The title compound was prepared from tert-butyl 4-hydroxyisoindoline-2-carboxylate (step 1b), CMBP and tetrahydrofuran-3-ol (CAS: 453-20-3) analogously to Intermediate YD step 1.

¹H NMR (DMSO-d6, 400 MHz): δ 1.45 (d, J=2.8 Hz, 9H), 1.94-1.99 (m, 1H), 2.18-2.25 (m, 1H), 3.73-3.89 (m, 4H), 4.44 (d, J=8.4 Hz, 2H), 4.57 (d, J=10.8 Hz, 2H), 5.08 (s, 1H), 6.86 (m, 2H), 7.25 (t, J=8 Hz, J=15.6 Hz, 1H).

LCMS (Method N): 1.84 min, MS: ES+ 205.9 (M−100).

### Step 2: 4-((Tetrahydrofuran-3-yl)oxy)isoindoline hydrochloride

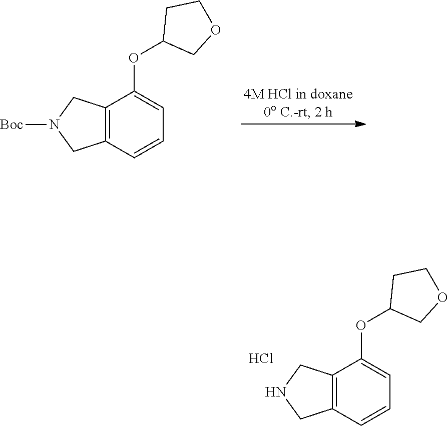 [SMILES: CC(C)(C)OC(=O)N1Cc2cccc(OC3CCOC3)c2C1 ; Cl ; c1cc2c(c(OC3CCOC3)c1)CNC2]

The title compound was prepared from tert-butyl 4-((tetrahydrofuran-3-yl)oxy)isoindoline-2-carboxylate (step 1) and 4M HCl in dioxane analogously to Intermediate YD step 2.

¹H NMR (DMSO-d6, 400 MHz): δ: 1.93-1.99 (m, 1H), 2.20-2.29 (m, 1H), 3.74-3.92 (m, 4H), 4.36 (s, 2H), 4.48 (s, 2H), 5.10 (s, 1H), 6.97 (t, J=17.2 Hz, J=17.2 Hz, 2H), 7.34 (t, J=7.6 Hz, J=15.6 Hz, 1H), 10.06 (s, 1H).

LCMS (Method N): 0.91 min, MS: ES+ 205.9 (M+1).

### Intermediate ZW: 8-((Tetrahydrofuran-3-yl)oxy)-1,2,3,4-tetrahydroisoquinoline hydrochloride

### Step 1: 1,2,3,4-Tetrahydroisoquinolin-8-ol

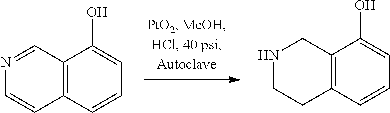 [SMILES: Oc1cccc2ccncc12 ; Oc1cccc2c1CNCC2]

To a stirred solution of isoquinolin-8-ol (CAS: 2439-04-5) (1 g, 6.88 mmol, 1.0 eq.) in MeOH (20 mL) were added PtO₂ (0.15 g, 0.62 mmol, 0.09 eq.) and HCl (0.5 mL) at room temperature. The reaction mixture was placed in an Autoclave under a pressure of 40 psi pressure of hydrogen gas at room temperature for 24 h. The resulting mixture was filtered through Celite® (filter material) and the filtrate was concentrated under vacuum yielding 1,2,3,4-tetrahydroisoquinolin-8-ol as a brown solid (0.75 g, Yield: 64%).

¹H NMR (DMSO-d6, 400 MHz): δ 2.56 (t, J=6 Hz, J=3.6 Hz, 2H), 3.05 (t, J=6 Hz, J=12 Hz, 2H), 3.89 (s, 2H), 5.76 (s, 1H), 6.50 (d, J=7.6 Hz, 1H), 6.64 (d, J=8 Hz, 1H), 6.93 (t, J=7.6 Hz, J=15.6 Hz, 1H), 9.36 (s, 1H).

LCMS (Method N): 0.40 min, MS: ES+ 149.7 (M+1).

### Step 2: tert-Butyl 8-hydroxy-3,4-dihydroisoquinoline-2(1H)-carboxylate

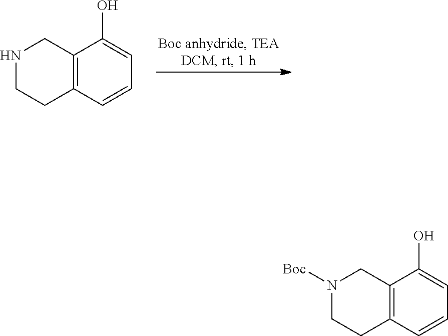 [SMILES: Oc1cccc2c1CNCC2 ; CC(C)(C)OC(=O)N1CCc2cccc(O)c2C1]

The title compound was prepared from 1,2,3,4-tetrahydroisoquinolin-8-ol (step 1), boc anhydride and TEA analogously to Intermediate N step 1.

¹H NMR (DMSO-d6, 400 MHz): δ 1.42 (s, 9H), 2.59 (t, J=5.6 Hz, J=11.6 Hz, 2H), 3.53 (t, J=5.6 Hz, J=11.6 Hz, 2H), 4.43 (s, 2H), 6.58 (d, J=7.6 Hz, 1H), 6.64 (d, J=8 Hz, 1H), 6.97 (t, J=7.6 Hz, J=15.6 Hz, 1H), 9.42 (s, 1H).

LCMS (Method N): 1.62 min, MS: ES+ 149.8 (M−100).

### Step 3: tert-Butyl 8-((tetrahydrofuran-3-yl)oxy)-3,4-dihydroisoquinoline-2(1H)-carboxylate

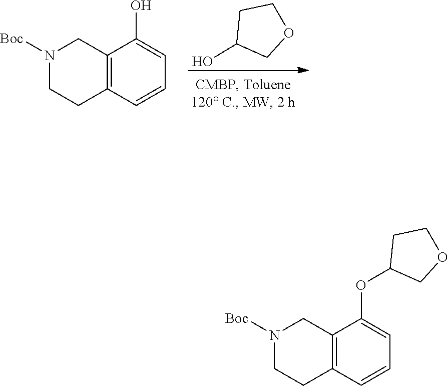

The title compound was prepared from tert-butyl 8-hydroxy-3,4-dihydroisoquinoline-2(1H)-carboxylate (step 2), CMBP and tetrahydrofuran-3-ol (CAS: 453-20-3) analogously to Intermediate YD step 1.

¹H NMR (DMSO-d6, 400 MHz): δ 1.42 (m, 9H), 1.55-1.62 (m, 2H), 2.60 (t, J=6 Hz, J=11.6 Hz, 2H), 3.53 (s, 2H), 3.74-3.92 (m, 4H), 4.46 (s, 2H), 5.02 (t, J=6 Hz, J=10.8 Hz, 1H), 6.74-6.80 (m, 2H), 7.13 (t, J=7.6 Hz, J=15.6 Hz, 1H).

LCMS (Method N): 1.91 min, MS: ES+ 219.9 (M−100).

### Step 4: 8-((Tetrahydrofuran-3-yl)oxy)-1,2,3,4-tetrahydroisoquinoline hydrochloride

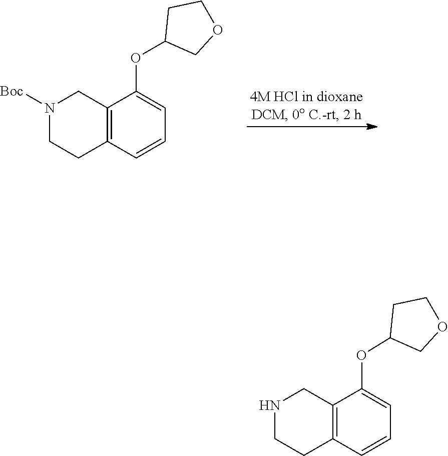 [SMILES: CC(C)(C)OC(=O)N1CCc2cccc(OC3CCOC3)c2C1 ; c1cc2c(c(OC3CCOC3)c1)CNCC2]

The title compound was prepared from tert-butyl 8-((tetrahydrofuran-3-yl)oxy)-3,4-dihydroisoquinoline-2(1H)-carboxylate (step 3) and 4M HCl in dioxane analogously to Intermediate YD step 2.

LCMS (Method O): 1.45 min, MS: ES+220.2 (M+1).

### Assay Protocols

### Fluorescence Polarisation Assay for MLH1

Test compounds, as 10 mM DMSO stocks, were dispensed into a 384-well Low Flange Black Flat Bottom Polystyrene Non-binding Surface plate (Corning®, item number 3575) using a Labcyte Echo acoustic liquid handler. Test compounds were added to wells in columns 1-22 whilst DMSO was added to wells in columns 23 and 24 to normalise the plate. 20 μL of a 2× solution (200 nM) of recombinant N-terminal MLH1 (residues 15-340) in assay buffer (25 mM HEPES, pH 7.5, 250 mM NaCl, 10 mM MgCl₂, 0.01% Triton X-100, 5 mM Dithiothreitol) was added to all wells in columns 1-23 and 20 μL assay buffer was added to all wells in column 24 using an E1-ClipTip pipette (ThermoFisher). Plates were centrifuged for 1 minute at 250 xg and were incubated at room temperature for 30 minutes prior to the addition of 20 μL of 2×(10 nM) of 5-((5-(4-((2-(2-(Benzyloxy)-4,6-dihydroxybenzoyl)isoindolin-5-yl)methyl)piperazin-1-yl)pentyl)carbamoyl)-2-(6-(dimethylamino)-3-(dimethyliminio)-3H-xanthen-9-yl)benzoate (referred to hereinafter as “probe compound”) in assay buffer (prepared from a 1 mM DMSO stock) with an E1-ClipTip pipette (ThermoFisher). The final concentration of N-terminal MLH1 was 100 nM and the final concentration of the probe compound was 5 nM. Compound plates were centrifuged for 1 minute at 250 xg for 1 minute and immediately read on a PheraStar FSX (fitted with 384-well aperture spoon and 590 675 675 FP optic module). The gain and focus were adjusted before each plate was read so that the polarisation of a no enzyme control (column 24) was equal to 35 mP. Data were normalised against the no inhibitor controls (column 23) and no enzyme controls (column 24).

### Fluorescence Polarisation Assay for PMS2

Test compounds, as 10 mM DMSO stocks, were dispensed into a Black Fluotrac 200 384 well medium binding plate (Greiner Bio-One, item number 781076) using a Labcyte Echo acoustic liquid handler. For single point screening, test compounds were added to wells in columns 1-22 whilst DMSO was added to wells in columns 23 and 24 to normalise the plate. For potency determination, serial dilutions of test compounds were added to wells in columns 3-22 and DMSO volume was normalised across the plate. 20 μL of a 2× solution (20 nM) of recombinant N-terminal PMS2 (residues 1-365) in assay buffer (25 mM HEPES, pH 7.5, 250 mM NaCl, 10 mM MgCl₂, 0.01% Triton X-100, 5 mM Dithiothreitol) was added to all wells in columns 2-23 for potency determination or columns 1-23 for single point screening. 20 μL assay buffer was added to all wells in columns 1 and 24 (column 24 only for single point screening) using a MultiDrop Combi (ThermoFisher). Plates were centrifuged for 1 minute at 250 xg and were incubated at room temperature for 30 minutes prior to the addition of 20 μL of 2×(20 nM) of 5-((5-(4-((2-(2,4-dihydroxy-5-isopropylbenzoyl) isoindolin-5-yl) methyl)piperazin-1-yl) pentyl)carbamoyl)-2-(6-(dimethylamino)-3-(dimethyliminio)-3H-xanthen-9-yl)benzoate (referred to hereinafter as “probe compound”) in assay buffer (prepared from a 100 μM DMSO stock) with a MultiDrop Combi (ThermoFisher). The final concentration of N-terminal PMS2 was 10 nM and the final concentration of probe compound was 5 nM. Compound plates were centrifuged for 1 minute at 250 xg for 1 minute and were incubated at room temperature for 1 hour before being read on a PheraStar FSX (fitted with 384-well aperture spoon and 540 590 590 FP optic module). The gain and focus were adjusted before each plate was read so that the polarisation of a no enzyme control (column 24) was equal to 35 mP. Data were normalised against the no inhibitor controls (column 23) and no enzyme controls (column 24). Compounds demonstrated IC₅₀>100 uM in this assay unless otherwise stated

### Fluorescence Polarisation Assay for HSP90

Test compounds, solubilised to a concentration of 10 mM in DMSO, were dispensed into a Black Fluotrac 200 384 well medium binding plates (Greiner Bio-One, item number 781076) using a Labcyte Echo acoustic liquid handler. For single point screening, test compounds were added to wells in columns 1-22 whilst for potency determination, serial dilutions of test compounds were added to wells in columns 3-22; in all cases, DMSO volume was normalised across the entire plate. All subsequent liquid handling steps were conducted using a Multidrop Combi (Thermo Scientific) or Matrix electronic pipettors (Thermo Scientific).

20 μL of a 2× solution (60 nM) of recombinant HSP90α (full length with an N-terminal His₆-tag, BPS Bioscience) in assay buffer (20 mM HEPES, pH 7.5, 100 mM NaCl, 10 mM MgCl₂, 0.01% Triton X-100, 1 mM Dithiothreitol) was added to all wells in columns 2-23 for potency determination or columns 1-23 for single point screening. 20 μL assay buffer was added to all wells in columns 1 and 24 (column 24 only for single point screening). Plates were centrifuged for 1 minute at 250 xg and were incubated at room temperature for 30 minutes prior to the addition of 20 μL of 2×(10 nM) of FITC-Geldanymycin in assay buffer (Abcam ab141589, prepared from a 100 μM DMSO stock). The final concentration of HSP90α was 30 nM and the final concentration of FITC-Geldanymycin was 5 nM.

Compound plates were centrifuged for 1 minute at 250 xg and were incubated at room temperature for 3 hours before being read on a PheraStar FSX (fitted with 384-well aperture spoon and 485 520 520 FP optic module). The gain and focus were adjusted before each plate was read so that the polarisation of a no enzyme control (column 24) was equal to 35 mP. Data were normalised against the no inhibitor controls (column 23) and no enzyme controls (column 24). Compounds demonstrated IC₅₀>100 uM in this assay unless otherwise stated.

### Assay Data Table

Data obtained in the above assays is shown in the Table below:
*1-10 μM
**0.1-1 μM
***less than 0.1 μM
PMS2 and HSP90 activity is >10 uM unless otherwise stated.

| Example | | MLH1 | PMS2 | HSP90 |
|---|---|---|---|---|
| no | Name | IC₅₀ | IC₅₀ | IC₅₀ |
| 1 | (S)-(2-(Benzyloxy)-6-hydroxy-4-methoxyphenyl)(4- | ** | | |
| | ((tetrahydrofuran-3-yl)amino)isoindolin-2-yl)methanone | | | |
| 2 | (R)-(2-(Benzyloxy)-6-hydroxy-4-methoxyphenyl)(4- | ** | | |
| | ((tetrahydrofuran-3-yl)amino)isoindolin-2-yl)methanone | | | |
| 3 | (R)-(2-(Benzyloxy)-4-(difluoromethyl)-6-hydroxyphenyl)(4- | ** | | |
| | ((tetrahydrofuran-3-yl)amino)isoindolin-2-yl)methanone | | | |
| 4 | (S)-(2-(Benzyloxy)-4-(difluoromethyl)-6-hydroxyphenyl)(4- | ** | | |
| | ((tetrahydrofuran-3-yl)amino)isoindolin-2-yl)methanone | | | |
| 4.1 | (S)-(4-(Difluoromethyl)-2-hydroxy-6-((3- | ** | | |
| | methoxybenzyl)oxy)phenyl)(4-((tetrahydrofuran-3- | | | |
| | yl)amino)isoindolin-2-yl)methanone | | | |
| 4.2 | (S)-(2-(Benzyloxy)-4-(difluoromethyl)-6-hydroxyphenyl)(6- | ** | | |
| | ((dimethylamino)methyl)-4-((tetrahydrofuran-3- | | | |
| | yl)amino)isoindolin-2-yl)methanone | | | |
| 4.3 | 1-(3-((2-(2-(Benzyloxy)-4-(difluoromethyl)-6- | ** | | |
| | hydroxybenzoyl)isoindolin-4-yl)amino)azetidin-1-yl)ethan-1- | | | |
| | one | | | |
| 4.4 | (2-(Benzyloxy)-4-(difluoromethyl)-6-hydroxyphenyl)(4- | ** | | |
| | (oxetan-3-ylamino)isoindolin-2-yl)methanone | | | |
| 4.5 | (4-(Difluoro methyl)-2-hydroxy-6-(pyridin-2-ylmethoxy) | ** | | |
| | phenyl) (4-(oxetan-3-ylamino) isoindolin-2-yl) methanone | | | |
| 4.6 | (S)-(2-(Benzyloxy)-4-(difluoromethyl)-6-hydroxyphenyl)(8- | ** | | |
| | ((tetrahydrofuran-3-yl)amino)-3,4-dihydroisoquinolin-2(1H)- | | | |
| | yl)methanone | | | |
| 4.7 | (2-(Benzyloxy)-4-(difluoromethyl)-6-hydroxyphenyl)(8- | ** | | |
| | (oxetan-3-ylamino)-3,4-dihydroisoquinolin-2(1H)-yl) | | | |
| | methanone | | | |
| 4.8 | (S)-(4-(Difluoromethyl)-2-hydroxy-6-((4- | ** | | |
| | methoxybenzyl)oxy)phenyl)(4-((tetrahydrofuran-3- | | | |
| | yl)amino)isoindolin-2-yl)methanone | | | |
| 4.9 | (S)-(4-(Difluoro methyl)-2-hydroxy-6-(pyridin-2-ylmethoxy) | ** | | |
| | phenyl) (4-((tetrahydrofuran-3-yl) amino) isoindolin-2-yl) | | | |
| | methanone | | | |
| 4.10 | (S)-(4-(Difluoromethyl)-2-hydroxy-6-(pyridin-2- | ** | | |
| | ylmethoxy)phenyl)(8-((tetrahydrofuran-3-yl)amino)-3,4- | | | |
| | dihydroisoquinolin-2(1H)-yl)methanone | | | |
| 4.11 | (S)-(4-(Difluoromethyl)-2-hydroxy-6-((4- | ** | | |
| | methoxybenzyl)oxy)phenyl)(8-((tetrahydrofuran-3-yl)amino)- | | | |
| | 3,4-dihydroisoquinolin-2(1H)-yl)methanone | | | |
| 4.12 | (S)-3-((2-(2-(Benzyloxy)-4-(difluoromethyl)-6- | ** | | |
| | hydroxybenzoyl)isoindolin-4-yl)amino)-1-methylpyrrolidin-2- | | | |
| | one | | | |
| 4.13 | (S)-3-((2-(2-(benzyloxy)-4-(difluoromethyl)-6- | ** | | |
| | hydroxybenzoyl)-1,2,3,4-tetrahydroisoquinolin-8-yl)amino)-1- | | | |
| | methylpyrrolidin-2-one | | | |
| 4.14 | (R)-3-((2-(2-(Benzyloxy)-4-(difluoromethyl)-6- | ** | | |
| | hydroxybenzoyl)isoindolin-4-yl)amino)-1-methylpyrrolidin-2- | | | |
| | one | | | |
| 4.15 | (R)-3-((2-(2-(Benzyloxy)-4-(difluoromethyl)-6- | ** | | |
| | hydroxybenzoyl)-1,2,3,4-tetrahydroisoquinolin-8-yl)amino)-1- | | | |
| | methylpyrrolidin-2-one | | | |
| 4.16 | 3-((2-(2-(Benzyloxy)-4-(difluoromethyl)-6- | ** | | |
| | hydroxybenzoyl)isoindolin-4-yl)amino)dihydrofuran-2(3H)- | | | |
| | one | | | |
| 4.17 | 3-((2-(2-(Benzyloxy)-4-(difluoromethyl)-6-hydroxybenzoyl)- | ** | | |
| | 1,2,3,4-tetrahydroisoquinolin-8-yl) amino) dihydrofuran- | | | |
| | 2(3H)-one | | | |
| 4.18 | (S)-(2-(Benzyloxy)-4-(difluoromethyl)-6-hydroxyphenyl)(6- | ** | | |
| | methoxy-4-((tetrahydrofuran-3-yl)amino)isoindolin-2- | | | |
| | yl)methanone | | | |
| 4.19 | (S)-(6-((1H-Imidazol-1-yl) methyl)-4-((tetrahydrofuran-3-yl) | ** | | |
| | amino) isoindolin-2-yl) (2-(benzyloxy)-4-(difluoromethyl)-6- | | | |
| | hydroxyphenyl)methanone | | | |
| 4.20 | (S)-(2-(Benzyloxy)-4-(difluoromethyl)-6-hydroxyphenyl)(6- | ** | | |
| | methoxy-8-((tetrahydrofuran-3-yl)amino)-3,4- | | | |
| | dihydroisoquinolin-2(1H)-yl)methanone | | | |
| 4.21 | (S)-(2-(Benzyloxy)-4-(difluoromethyl)-6-hydroxyphenyl)(6- | *** | | |
| | ((dimethylamino)methyl)-8-((tetrahydrofuran-3-yl)amino)-3,4- | | | |
| | dihydroisoquinolin-2(1H)-yl)methanone | | | |
| 4.22 | (S)-(6-((1H-Imidazol-1-yl)methyl)-8-((tetrahydrofuran-3- | *** | | |
| | yl)amino)-3,4-dihydroisoquinolin-2(1H)-yl)(2-(benzyloxy)-4- | | | |
| | (difluoromethyl)-6-hydroxyphenyl)methanone | | | |
| 4.23 | (S)-(2-(Benzyloxy)-4-(difluoromethyl)-6-hydroxyphenyl)(7- | * | | |
| | methoxy-8-((tetrahydrofuran-3-yl)amino)-3,4- | | | |
| | dihydroisoquinolin-2(1H)-yl)methanone | | | |
| 4.24 | (S)-(2-(Benzyloxy)-4-(difluoromethyl)-6-hydroxyphenyl)(6-(1- | ** | | |
| | methyl-1H-imidazol-5-yl)-4-((tetrahydrofuran-3- | | | |
| | yl)amino)isoindolin-2-yl)methanone | | | |
| 4.25 | (S)-(2-(Benzyloxy)-4-(difluoromethyl)-6-hydroxyphenyl)(6- | ** | | |
| | (1-methyl-1H-imidazol-4-yl)-4-((tetrahydrofuran-3- | | | |
| | yl)amino)isoindolin-2-yl)methanone | | | |
| 4.26 | (S)-(2-(Benzyloxy)-4-(difluoromethyl)-6-hydroxyphenyl) (6- | ** | | |
| | (1-methyl-1H-imidazol-5-yl)-8-((tetrahydrofuran-3-yl) amino)- | | | |
| | 3,4-dihydroisoquinolin-2(1H)-yl)methanone | | | |
| 4.27 | (S)-(2-(Benzyloxy)-4-(difluoromethyl)-6-hydroxyphenyl) (6- | ** | | |
| | (1-methyl-1H-imidazol-4-yl)-8-((tetrahydrofuran-3-yl) amino)- | | | |
| | 3,4-dihydroisoquinolin-2(1H)-yl)methanone | | | |
| 4.28 | 2-(Benzyloxy)-4-(difluoromethyl)-6-hydroxyphenyl)(4-((2- | ** | | |
| | methoxyethyl)amino)isoindolin-2-yl)methanone | | | |
| 4.29 | 2-((2-(2-(Benzyloxy)-4-(difluoro methyl)-6-hydroxybenzoyl) | ** | | |
| | isoindolin-4-yl) amino)-N, N-dimethylacetamide | | | |
| 4.30 | (2-(Benzyloxy)-4-(difluoromethyl)-6-hydroxyphenyl) (5-((4- | ** | | |
| | methylpiperazin-1-yl) methyl) isoindolin-2-yl) methanone | | | |
| 4.31 | (2-(Benzyloxy)-4-(difluoromethyl)-6-hydroxyphenyl)(5-(2- | ** | | |
| | (dimethylamino)ethoxy)isoindolin-2-yl)methanone | | | |
| 4.32 | (2-(Benzyloxy)-4-(difluoromethyl)-6-hydroxyphenyl)(5-((1- | *** | * | * |
| | methylpiperidin-4-yl)oxy)isoindolin-2-yl)methanone | | | |
| 4.33 | ((2-(benzyloxy)-4-(difluoromethyl)-6-hydroxyphenyl)(4-((1- | ** | | |
| | methylazetidin-3-yl)amino)isoindolin-2-yl)methanone | | | |
| 4.34 | (2-(Benzyloxy)-4-(difluoro methyl)-6-hydroxyphenyl) (4- | * | | |
| | (methylamino) isoindolin-2-yl) methanone | | | |
| 4.35 | (2-(Benzyloxy)-4-(difluoromethyl)-6-hydroxyphenyl)(3,4- | * | | |
| | dihydroisoquinolin-2(1H)-yl)methanone | | | |
| 4.36 | (2-(Benzyloxy)-4-(difluoromethyl)-6-hydroxyphenyl)(8- | * | | |
| | (methylamino)-3,4-dihydroisoquinolin-2(1H)-yl)methanone | | | |
| 4.37 | (2-(Benzyloxy)-4-(difluoromethyl)-6-hydroxyphenyl)(8-((2- | * | | |
| | methoxyethyl)amino)-3,4-dihydroisoquinolin-2(1H)- | | | |
| | yl)methanone | | | |
| 4.38 | (2-(Benzyloxy)-4-(difluoromethyl)-6-hydroxyphenyl)(7- | * | | |
| | methoxy-3,4-dihydroisoquinolin-2(1H)-yl)methanone | | | |
| 4.39 | (2-(Benzyloxy)-4-(difluoromethyl)-6-hydroxyphenyl)(6- | * | | |
| | methoxy-3,4-dihydroisoquinolin-2(1H)-yl)methanone | | | |
| 4.40 | (2-(Benzyloxy)-4-(difluoromethyl)-6-hydroxyphenyl)(7-(2- | ** | | |
| | (dimethylamino)ethoxy)-3,4-dihydroisoquinolin-2(1H)- | | | |
| | yl)methanone | | | |
| 4.41 | (2-(Benzyloxy)-4-s(difluoromethyl)-6-hydroxyphenyl)(6-(2- | ** | ** | |
| | (dimethylamino)ethoxy)-3,4-dihydroisoquinolin-2(1H)- | | | |
| | yl)methanone | | | |
| 4.42 | (2-(Benzyloxy)-4-(difluoromethyl)-6-hydroxyphenyl)(7- | ** | | |
| | ((dimethylamino)methyl)-3,4-dihydroisoquinolin-2(1H)- | | | |
| | yl)methanone | | | |
| 4.43 | (2-(Benzyloxy)-4-(difluoromethyl)-6-hydroxyphenyl)(6- | ** | | |
| | ((dimethylamino)methyl)-3,4-dihydroisoquinolin-2(1H)- | | | |
| | yl)methanone | | | |
| 4.44 | (2-(Benzyloxy)-4-(difluoromethyl)-6-hydroxyphenyl)(7-((1- | ** | | |
| | methylpiperidin-4-yl)oxy)-3,4-dihydroisoquinolin-2(1H)- | | | |
| | yl)methanone | | | |
| 4.45 | (2-(Benzyloxy)-4-(difluoromethyl)-6-hydroxyphenyl)(6-((1- | ** | | |
| | methylpiperidin-4-yl)oxy)-3,4-dihydroisoquinolin-2(1H)- | | | |
| | yl)methanone | | | |
| 4.46 | (2-(Benzyloxy)-4-(difluoromethyl)-6-hydroxyphenyl)(7-((4- | ** | | |
| | methylpiperazin-1-yl)methyl)-3,4-dihydroisoquinolin-2(1H)- | | | |
| | yl)methanone | | | |
| 4.47 | (2-(Benzyloxy)-4-(difluoromethyl)-6-hydroxyphenyl)(6-((4- | ** | | |
| | methylpiperazin-1-yl)methyl)-3,4-dihydroisoquinolin-2(1H)- | | | |
| | yl)methanone | | | |
| 4.48 | (7-((1H-Imidazol-1-yl)methyl)-3,4-dihydroisoquinolin-2(1H)- | ** | | |
| | yl)(2-(benzyloxy)-4-(difluoromethyl)-6- | | | |
| | hydroxyphenyl)methanone | | | |
| 4.49 | (6-((1H-Imidazol-1-yl)methyl)-3,4-dihydroisoquinolin-2(1H)- | ** | | |
| | yl)(2-(benzyloxy)-4-(difluoromethyl)-6- | | | |
| | hydroxyphenyl)methanone | | | |
| 4.50 | (5-((1H-Imidazol-1-yl)methyl)isoindolin-2-yl)(2-(benzyloxy)-4- | ** | | |
| | (difluoromethyl)-6-hydroxyphenyl)methanone | | | |
| 4.51 | (2-(Benzyloxy)-4-(difluoromethyl)-6-hydroxyphenyl)(5- | ** | | |
| | ((dimethylamino)methyl)isoindolin-2-yl)methanone | | | |
| 4.52 | (2-(Benzyloxy)-4-(difluoromethyl)-6-hydroxyphenyl)(2- | ** | | |
| | ((dimethylamino)methyl)-5,7-dihydro-6H-pyrrolo[3,4- | | | |
| | b]pyridin-6-yl)methanone | | | |
| 4.53 | (2-(Benzyloxy)-4-(difluoromethyl)-6-hydroxyphenyl)(3- | * | | |
| | ((dimethylamino)methyl)-5,7-dihydro-6H-pyrrolo[3,4- | | | |
| | b]pyridin-6-yl)methanone | | | |
| 4.54 | (2-(Benzyloxy)-4-(difluoromethyl)-6- | * | | |
| | hydroxyphenyl)(isoindolin-2-yl)methanone | | | |
| 4.55 | (R)-(2-(Benzyloxy)-4-(difluoromethyl)-6-hydroxyphenyl)(5- | *** | | |
| | ((1-methylpyrrolidin-3-yl)oxy)isoindolin-2-yl)methanone | | | |
| 4.56 | (S)-(2-(Benzyloxy)-4-(difluoromethyl)-6-hydroxyphenyl)(5- | *** | * | |
| | ((1-methylpyrrolidin-3-yl)oxy)isoindolin-2-yl)methanone | | | |
| 4.57 | 2-(Benzyloxy)-4-(difluoromethyl)-6-hydroxyphenyl)(5-((1- | *** | | |
| | methylazetidin-3-yl)oxy)isoindolin-2-yl)methanone | | | |
| 4.58 | (4-(Difluoromethyl)-2-hydroxy-6-(pyridin-2- | ** | | |
| | ylmethoxy)phenyl)(5-((1-methylpiperidin-4-yl)oxy)isoindolin- | | | |
| | 2-yl)methanone | | | |
| 4.59 | (2-(Cyclopropylmethoxy)-4-(difluoromethyl)-6- | ** | | * |
| | hydroxyphenyl)(5-((1-methylpiperidin-4-yl)oxy)isoindolin-2- | | | |
| | yl)methanone | | | |
| 4.60 | (2-(Benzyloxy)-4-(difluoromethyl)-6-hydroxyphenyl)(5- | ** | | |
| | (pyridin-4-yloxy)isoindolin-2-yl)methanone | | | |
| 4.61 | (2-(Cyclopropylmethoxy)-4-(difluoromethyl)-6- | ** | | |
| | hydroxyphenyl)(5-((1-methylpiperidin-4-yl)oxy)isoindolin-2- | | | |
| | yl)methanone | | | |
| 4.62 | (S)-(2-(Benzyloxy)-4-(difluoromethyl)-6-hydroxyphenyl)(6- | *** | | |
| | ((1-methylpiperidin-4-yl)oxy)-4-((tetrahydrofuran-3- | | | |
| | yl)amino)isoindolin-2-yl)methanone | | | |
| 4.63 | (R)-(2-(Benzyloxy)-4-(difluoromethyl)-6-hydroxyphenyl)(6- | ** | | |
| | ((1-methylpiperidin-4-yl)oxy)-4-((tetrahydrofuran-3- | | | |
| | yl)amino)isoindolin-2-yl)methanone | | | |
| 4.64 | (S)-(2-(Benzyloxy)-4-(difluoromethyl)-6-hydroxyphenyl)(6- | ** | | |
| | ((1-methylpyrrolidin-3-yl)oxy)-3,4-dihydroisoquinolin-2(1H) | | | |
| | yl)methanone | | | |
| 4.65 | (R)-(2-(Benzyloxy)-4-(difluoromethyl)-6-hydroxyphenyl)(6- | ** | | |
| | ((1-methylpyrrolidin-3-yl)oxy)-3,4-dihydroisoquinolin-2(1H) | | | |
| | yl)methanone | | | |
| 4.66 | (2-(Benzyloxy)-4-(difluoromethyl)-6-hydroxyphenyl)(6-((1- | ** | | |
| | methylazetidin-3-yl)oxy)-3,4-dihydroisoquinolin-2(1H)- | | | |
| | yl)methanone | | | |
| 4.67 | (R)-(2-(Benzyloxy)-4-(difluoromethyl)-6-hydroxyphenyl)(7- | ** | | |
| | ((1-methylpyrrolidin-3-yl)oxy)-3,4-dihydroisoquinolin-2(1H)- | | | |
| | yl)methanone | | | |
| 4.68 | (S)-(2-(Benzyloxy)-4-(difluoromethyl)-6-hydroxyphenyl)(7- | ** | | |
| | ((1-methylpyrrolidin-3-yl)oxy)-3,4-dihydroisoquinolin-2(1H)- | | | |
| | yl)methanone | | | |
| 4.69 | (2-(Benzyloxy)-4-(difluoromethyl)-6-hydroxyphenyl)(7-((1- | ** | | |
| | methylazetidin-3-yl)oxy)-3,4-dihydroisoquinolin-2(1H)- | | | |
| | yl)methanone | | | |
| 4.70 | (2-(Benzyloxy)-4-(difluoro methyl)-6-hydroxyphenyl) (5-(2- | ** | | |
| | morpholinoethoxy) isoindolin-2-yl) methanone | | | |
| 4.71 | (2-(Benzyloxy)-4-(difluoromethyl)-6-hydroxyphenyl) (5-(2-(4- | ** | | |
| | methylpiperazin-1-yl) ethoxy) isoindolin-2-yl) methanone | | | |
| 4.72 | (2-(Benzyloxy)-4-(difluoromethyl)-6-hydroxyphenyl)(8- | *** | | |
| | (cyclopentylamino)-6-(2-(dimethylamino)ethoxy)-3,4- | | | |
| | dihydroisoquinolin-2(1H)-yl)methanone | | | |
| 4.73 | (2-(Benzyloxy)-4-(difluoromethyl)-6-hydroxyphenyl)(6-(2- | ** | | |
| | (dimethylamino)ethoxy)-8-(methylamino)-3,4- | | | |
| | dihydroisoquinolin-2(1H)-yl)methanone | | | |
| 4.74 | (2-(Benzyloxy)-4-(difluoromethyl)-6-hydroxyphenyl)(8- | ** | | |
| | (difluoromethyl)-6-(2-(dimethylamino)ethoxy)-3,4- | | | |
| | dihydroisoquinolin-2(1H)-yl)methanone | | | |
| 4.75 | (R)-(2-(Benzyloxy)-4-(difluoromethyl)-6-hydroxyphenyl)(6-(2- | *** | | |
| | (dimethylamino)ethoxy)-4-((tetrahydrofuran-3- | | | |
| | yl)amino)isoindolin-2-yl)methanone | | | |
| 4.76 | ((S)-(2-(Benzyloxy)-4-(difluoromethyl)-6-hydroxyphenyl)(6- | *** | | |
| | (2-(dimethylamino)ethoxy)-4-((tetrahydrofuran-3- | | | |
| | yl)amino)isoindolin-2-yl)methanone | | | |
| 4.77 | (2-(Benzyloxy)-4-(difluoromethyl)-6-hydroxyphenyl)(5-(2- | ** | | |
| | methoxyethoxy)isoindolin-2-yl)methanone | | | |
| 4.78 | (2-(Benzyloxy)-4-(difluoromethyl)-6-hydroxyphenyl)(5- | ** | | |
| | ((tetrahydro-2H-pyran-4-yl)oxy)isoindolin-2-yl)methanone | | | |
| 4.79 | (2-(Benzyloxy)-4-(difluoromethyl)-6-hydroxyphenyl)(4- | * | | |
| | ((tetrahydrofuran-3-yl)oxy)isoindolin-2-yl)methanone | | | |
| 4.80 | 2-(Benzyloxy)-4-(difluoromethyl)-6-hydroxyphenyl)(8- | * | | |
| | ((tetrahydrofuran-3-yl)oxy)-3,4-dihydroisoquinolin-2(1H)- | | | |
| | yl)methanone | | | |
| 5 | (S)-(2-(Benzyloxy)-4-fluoro-6-hydroxyphenyl)(4- | ** | | |
| | ((tetrahydrofuran-3-yl)amino)isoindolin-2-yl)methanone | | | |
| 6 | (R)-(2-(Benzyloxy)-4-fluoro-6-hydroxyphenyl)(4- | * | | |
| | ((tetrahydrofuran-3-yl)amino)isoindolin-2-yl)methanone | | | |
| 7 | (R)-(2-(Benzyloxy)-4-chloro-6-hydroxyphenyl)(4- | * | | |
| | ((tetrahydrofuran-3-yl)amino)isoindolin-2-yl)methanone | | | |
| 8 | (S)-(2-(Benzyloxy)-4-chloro-6-hydroxyphenyl)(4- | ** | | |
| | ((tetrahydrofuran-3-yl)amino)isoindolin-2-yl)methanone | | | |
| 9 | (R)-(2-(Benzyloxy)-4-chloro-6-hydroxyphenyl)(6-(2- | ** | | |
| | (dimethylamino)ethoxy)-4-((tetrahydrofuran-3- | | | |
| | yl)amino)isoindolin-2-yl)methanone | | | |
| 10 | (S)-(2-(benzyloxy)-4-chloro-6-hydroxyphenyl)(6-(2- | *** | | |
| | (dimethylamino)ethoxy)-4-((tetrahydrofuran-3- | | | |
| | yl)amino)isoindolin-2-yl)methanone | | | |
| 10.1 | (R)-(2-(Benzyloxy)-4-chloro-6-hydroxyphenyl)(6-methoxy-4- | * | | |
| | ((tetrahydrofuran-3-yl)amino)isoindolin-2-yl)methanone | | | |
| 10.2 | (S)-(2-(benzyloxy)-4-chloro-6-hydroxyphenyl)(6-methoxy-4- | ** | | |
| | ((tetrahydrofuran-3-yl)amino)isoindolin-2-yl)methanone | | | |
| 10.3 | 3-(Benzyloxy)-5-hydroxy-4-(4-((1-methyl-5-oxopyrrolidin-3- | ** | | |
| | yl)amino)isoindoline-2-carbonyl)benzonitrile | | | |
| 10.4 | 3-(Benzyloxy)-5-hydroxy-4-(4-(pyrimidin-5- | * | | |
| | ylamino)isoindoline-2-carbonyl)benzonitrile | | | |
| 10.5 | (R)-4-(4-((1-Acetylpyrrolidin-3-yl)amino)isoindoline-2- | ** | | |
| | carbonyl)-3-(benzyloxy)-5-hydroxybenzonitrile | | | |
| 10.6 | (S)-4-(4-((1-acetylpyrrolidin-3-yl)amino)isoindoline-2- | ** | | |
| | carbonyl)-3-(benzyloxy)-5-hydroxybenzonitrile | | | |
| 10.7 | (R)-3-(Benzyloxy)-5-hydroxy-4-(4-((1-methyl-5-oxopyrrolidin- | ** | | |
| | 3-yl)amino)isoindoline-2-carbonyl)benzonitrile or (S)-3- | | | |
| | (benzyloxy)-5-hydroxy-4-(4-((1-methyl-5-oxopyrrolidin-3- | | | |
| | yl)amino)isoindoline-2-carbonyl)benzonitrile | | | |
| 10.8 | (R)-3-(Benzyloxy)-5-hydroxy-4-(4-((1-methyl-5-oxopyrrolidin- | ** | | |
| | 3-yl)amino)isoindoline-2-carbonyl)benzonitrile or (S)-3- | | | |
| | (benzyloxy)-5-hydroxy-4-(4-((1-methyl-5-oxopyrrolidin-3- | | | |
| | yl)amino)isoindoline-2-carbonyl)benzonitrile | | | |
| 10.9 | 3-(Benzyloxy)-5-hydroxy-4-(4-((oxazol-4- | ** | | |
| | ylmethyl)amino)isoindoline-2-carbonyl)benzonitrile | | | |
| 10.10 | 3-(Benzyloxy)-5-hydroxy-4-(4-(((tetrahydrofuran-3- | * | | |
| | yl)methyl)amino)isoindoline-2-carbonyl)benzonitrile | | | |
| 10.11 | 3-(Benzyloxy)-5-hydroxy-4-(4-methoxyisoindoline-2- | * | | |
| | carbonyl)benzonitrile | | | |
| 10.12 | (R)-3-(Benzyloxy)-5-hydroxy-4-(8-((tetrahydrofuran-3- | * | | |
| | yl)amino)-1,2,3,4-tetrahydroisoquinoline-2- | | | |
| | carbonyl)benzonitrile | | | |
| 10.13 | (R)-3-(benzyloxy)-5-hydroxy-4-(6-methoxy-4- | ** | | |
| | ((tetrahydrofuran-3-yl)amino)isoindoline-2- | | | |
| | carbonyl)benzonitrile | | | |
| 10.14 | (S)-3-(Benzyloxy)-5-hydroxy-4-(6-methoxy-4- | ** | | |
| | ((tetrahydrofuran-3-yl)amino)isoindoline-2- | | | |
| | carbonyl)benzonitrile | | | |
| 10.15 | 3-(Benzyloxy)-4-(5-((dimethylamino)methyl)isoindoline-2- | * | | |
| | carbonyl)-5-hydroxybenzonitrile | | | |
| 10.16 | 4-(4-((1-Acetylpiperidin-4-yl)amino)isoindoline-2-carbonyl)-3- | ** | | |
| | (benzyloxy)-5-hydroxybenzonitrile | | | |
| 10.17 | (R)-4-(4-((1-Acetylpiperidin-3-yl)amino)isoindoline-2- | * | | |
| | carbonyl)-3-(benzyloxy)-5-hydroxybenzonitrile | | | |
| 10.18 | (S)-4-(4-((1-Acetylpiperidin-3-yl)amino)isoindoline-2- | ** | | |
| | carbonyl)-3-(benzyloxy)-5-hydroxybenzonitrile | | | |
| 10.19 | 3-(Benzyloxy)-5-hydroxy-4-(4-((5-oxotetrahydrofuran-3- | ** | | |
| | yl)amino)isoindoline-2-carbonyl)benzonitrile | | | |
| 10.20 | (2-(Benzyloxy)-4-chloro-6-hydroxyphenyl)(4-((1- | ** | | |
| | methylazetidin-3-yl)amino)isoindolin-2-yl)methanone | | | |
| 10.21 | 2-(Benzyloxy)-4-chloro-6-hydroxyphenyl)(2- | * | | |
| | ((dimethylamino)methyl)-5,7-dihydro-6H-pyrrolo[3,4- | | | |
| | b]pyridin-6-yl)methanone | | | |
| 10.22 | (2-(Benzyloxy)-4-chloro-6-hydroxyphenyl)(6-(2- | * | | |
| | (dimethylamino)ethoxy)-3,4-dihydroisoquinolin-2(1H)- | | | |
| | yl)methanone | | | |
| 10.23 | 1-(3-((2-(2-(Benzyloxy)-4-fluoro-6-hydroxybenzoyl)isoindolin- | ** | | |
| | 4-yl)amino)azetidin-1-yl)ethan-1-one | | | |
| 10.24 | (2-(Benzyloxy)-4-fluoro-6-hydroxyphenyl)(6-(2- | * | | |
| | (dimethylamino)ethoxy)-3,4-dihydroisoquinolin-2(1H)- | | | |
| | yl)methanone | | | |
| 10.25 | (2-(Benzyloxy)-4-fluoro-6-hydroxyphenyl) (5-((1- | ** | * | |
| | methylpiperidin-4-yl) oxy) isoindolin-2-yl) methadone | | | |
| 10.26 | 3-(Benzyloxy)-5-hydroxy-4-(4-(oxetan-3-ylamino)isoindoline- | ** | | |
| | 2-carbonyl)benzonitrile | | | |
| 10.27 | (S)-3-(Benzyloxy)-5-hydroxy-4-(4-((1-methyl-2-oxopyrrolidin- | ** | | |
| | 3-yl)amino)isoindoline-2-carbonyl)benzonitrile | | | |
| 10.28 | (R)-3-(benzyloxy)-5-hydroxy-4-(4-((1-methyl-2-oxopyrrolidin- | ** | | |
| | 3-yl)amino)isoindoline-2-carbonyl)benzonitrile | | | |
| 10.29 | 3-(Benzyloxy)-4-(6-(2-(dimethyl amino) ethoxy)-1,2,3,4- | * | | |
| | tetrahydroisoquinoline-2-carbonyl)-5-hydroxybenzonitrile | | | |
| 10.30 | (R)-3-(Benzyloxy)-5-hydroxy-4-(5-((1-methylpyrrolidin-3- | ** | | |
| | yl)oxy)isoindoline-2-carbonyl)benzonitrile | | | |
| 10.31 | (S)-3-(Benzyloxy)-5-hydroxy-4-(5-((1-methylpyrrolidin-3- | ** | | |
| | yl)oxy)isoindoline-2-carbonyl)benzonitrile | | | |
| 10.32 | 3-(Benzyloxy)-5-hydroxy-4-(5-((1-methylazetidin-3- | ** | | |
| | yl)oxy)isoindoline-2-carbonyl)benzonitrile | | | |
| 10.33 | 3-(Benzyloxy)-5-hydroxy-4-(5-((1-methylpiperidin-4- | ** | | |
| | yl)oxy)isoindoline-2-carbonyl)benzonitrile | | | |
| 10.34 | 3-(Benzyloxy)-5-hydroxy-4-(5-((4-methylpiperazin-1- | ** | | |
| | yl)methyl)isoindoline-2-carbonyl)benzonitrile | | | |
| 10.35 | 3-(Benzyloxy)-5-hydroxy-4-(5-(2-methoxyethoxy)isoindoline- | ** | | |
| | 2-carbonyl)benzonitrile | | | |
| 10.36 | 3-(Benzyloxy)-5-hydroxy-4-(5-((tetrahydro-2H-pyran-4- | ** | | |
| | yl)oxy)isoindoline-2-carbonyl)benzonitrile | | | |
| 10.37 | 3-Hydroxy-4-(5-((1-methylazetidin-3-yl)oxy)isoindoline-2- | * | | |
| | carbonyl)-5-(pyridin-2-ylmethoxy)benzonitrile | | | |
| 10.38 | 3-Hydroxy-4-(4-(oxetan-3-ylamino)isoindoline-2-carbonyl)-5- | * | | |
| | (pyridin-2-ylmethoxy)benzonitrile | | | |
| 10.39 | (S)-3-Hydroxy-5-(pyridin-2-ylmethoxy)-4-(4- | ** | | |
| | ((tetrahydrofuran-3-yl)amino)isoindoline-2- | | | |
| | carbonyl)benzonitrile | | | |
| 12 | (S)-3-(Benzyloxy)-5-hydroxy-4-(4-((tetrahydrofuran-3- | ** | | |
| | yl)amino)isoindoline-2-carbonyl)benzonitrile | | | |
| 12.1 | (S)-3-Hydroxy-5-methoxy-4-(4-((tetrahydrofuran-3- | * | | |
| | yl)amino)isoindoline-2-carbonyl)benzonitrile | | | |
| 12.2 | (S)-3-(Cyclopropylmethoxy)-5-hydroxy-4-(4- | * | | |
| | ((tetrahydrofuran-3-yl)amino)isoindoline-2- | | | |
| | carbonyl)benzonitrile | | | |
| 12.3 | (R)-3-(Benzyloxy)-5-hydroxy-4-(4-((tetrahydrofuran-3- | * | | |
| | yl)amino)isoindoline-2-carbonyl)benzonitrile | | | |
| 12.4 | 3-(Benzyloxy)-5-hydroxy-4-(isoindoline-2- | * | | |
| | carbonyl)benzonitrile | | | |
| 13 | 3-(Benzyloxy)-5-hydroxy-4-(5-methoxyisoindoline-2- | * | | |
| | carbonyl)benzonitrile | | | |
| 14 | (R)-3-(Benzyloxy)-4-(6-(2-(dimethylamino)ethoxy)-8- | ** | | |
| | ((tetrahydrofuran-3-yl)amino)-1,2,3,4-tetrahydroisoquinoline- | | | |
| | 2-carbonyl)-5-hydroxybenzonitrile | | | |
| 15 | (S)-3-(Benzyloxy)-4-(6-(2-(dimethylamino)ethoxy)-8- | *** | | |
| | ((tetrahydrofuran-3-yl)amino)-1,2,3,4-tetrahydroisoquinoline- | | | |
| | 2-carbonyl)-5-hydroxybenzonitrile | | | |
| 16 | 4-(4-((1-Acetylazetidin-3-yl)amino)isoindoline-2-carbonyl)-3- | ** | | |
| | (benzyloxy)-5-hydroxybenzonitrile | | | |
| 17 | (S)-3-(Benzyloxy)-5-hydroxy-4-(8-((tetrahydrofuran-3- | ** | | |
| | yl)amino)-1,2,3,4-tetrahydroisoquinoline-2- | | | |
| | carbonyl)benzonitrile | | | |
| 18 | (R)-3-(Benzyloxy)-4-(6-(2-(dimethylamino)ethoxy)-4- | ** | | |
| | ((tetrahydrofuran-3-yl)amino)isoindoline-2-carbonyl)-5- | | | |
| | hydroxybenzonitrile | | | |
| 19 | (S)-3-(Benzyloxy)-4-(6-(2-(dimethylamino)ethoxy)-4- | ** | | |
| | ((tetrahydrofuran-3-yl)amino)isoindoline-2-carbonyl)-5- | | | |
| | hydroxybenzonitrile | | | |
| 21 | (R)-(2-(Benzyloxy)-4-chloro-6-hydroxyphenyl)(6-(2- | *** | | |
| | (dimethylamino)ethoxy)-8-((tetrahydrofuran-3-yl)amino)-3,4- | | | |
| | dihydroisoquinolin-2(1H)-yl)methanone | | | |
| 22 | (S)-(2-(Benzyloxy)-4-chloro-6-hydroxyphenyl)(6-(2- | *** | | |
| | (dimethylamino)ethoxy)-8-((tetrahydrofuran-3-yl)amino)-3,4- | | | |
| | dihydroisoquinolin-2(1H)-yl)methanone | | | |
| 23 | (S)-(2-(Benzyloxy)-6-hydroxy-4-(trifluoromethyl) phenyl) (4- | ** | | |
| | ((tetrahydrofuran-3-yl) amino) isoindolin-2-yl) methanone | | | |
| 24 | (S)-(2-(Benzyloxy)-4-(difluoromethyl)-6-hydroxyphenyl)(6-(2- | *** | | |
| | (dimethylamino)ethoxy)-8-((tetrahydrofuran-3-yl)amino)-3,4- | | | |
| | dihydroisoquinolin-2(1H)-yl)methanone | | | |
| 25 | (R)-(2-(Benzyloxy)-4-(difluoromethyl)-6-hydroxyphenyl)(6-(2- | *** | | |
| | (dimethylamino)ethoxy)-8-((tetrahydrofuran-3-yl)amino)-3,4- | | | |
| | dihydroisoquinolin-2(1H)-yl)methanone | | | |
| 26 | (2-(Benzyloxy)-4-(difluoromethyl)-6-hydroxyphenyl)(5- | *** | * | |
| | (piperidin-4-yloxy)isoindolin-2-yl)methanone | | | |
| 27 | 3-(Benzyloxy)-4-(6-((dimethylamino)methyl)-2,3-dihydro-1H- | * | | |
| | pyrrolo[3,4-c]pyridine-2-carbonyl)-5-hydroxybenzonitrile | | | |
| 27.1 | 3-(Benzyloxy)-5-hydroxy-4-(6-(4-methylpiperazin-1-yl)-2,3- | * | | |
| | dihydro-1H-pyrrolo[3,4-c]pyridine-2-carbonyl)benzonitrile | | | |
| 27.2 | (2-(Benzyloxy)-4-(difluoromethyl)-6-hydroxyphenyl)(6- | ** | | |
| | ((tetrahydrofuran-3-yl)amino)indolin-1-yl)methanone | | | |
| 27.2a | (S or R)-(2-(Benzyloxy)-4-(difluoromethyl)-6- | ** | | |
| | hydroxyphenyl)(6-((tetrahydrofuran-3-yl)amino)indolin-1- | | | |
| | yl)methanone | | | |
| 27.2b | (S or R)-(2-(Benzyloxy)-4-(difluoromethyl)-6- | ** | | |
| | hydroxyphenyl)(6-((tetrahydrofuran-3-yl)amino)indolin-1- | | | |
| | yl)methanone or | | | |
| 27.3 | 3-(Benzyloxy)-5-hydroxy-4-(6-((tetrahydrofuran-3- | * | | |
| | yl)amino)indoline-1-carbonyl)benzonitrile | | | |
| 27.4 | 3-(Benzyloxy)-5-hydroxy-4-(5-((tetrahydrofuran-3- | * | | |
| | yl)amino)indoline-1-carbonyl)benzonitrile | | | |
| 27.5 | (2-(Benzyloxy)-4-(difluoromethyl)-6-hydroxyphenyl)(5- | * | | |
| | ((tetrahydrofuran-3-yl)amino)indolin-1-yl)methanone | | | |
| 27.5a | (S or R)-(2-(Benzyloxy)-4-(difluoromethyl)-6- | ** | | |
| | hydroxyphenyl)(5-((tetrahydrofuran-3-yl)amino)indolin-1- | | | |
| | yl)methanone | | | |
| 27.5b | (S or R)-(2-(Benzyloxy)-4-(difluoromethyl)-6- | ** | | |
| | hydroxyphenyl)(5-((tetrahydrofuran-3-yl)amino)indolin-1- | | | |
| | yl)methanone | | | |
| 27.6 | 3-(Benzyloxy)-5-hydroxy-4-(7-((1-methylpiperidin-4-yl)oxy)- | * | | |
| | 2,3,4,5-tetrahydro-1H-benzo[d]azepine-3- | | | |
| | carbonyl)benzonitrile hydroformate | | | |
| 27.7 | (2-(Benzyloxy)-4-(difluoromethyl)-6-hydroxyphenyl)(7-((1- | ** | | |
| | methylpiperidin-4-yl)oxy)-1,2,4,5-tetrahydro-3H- | | | |
| | benzo[d]azepin-3-yl)methanone | | | |
| 27.8 | 3-(Benzyloxy)-5-hydroxy-4-(6-morpholino-2,3-dihydro-1H- | * | | |
| | pyrrolo[3,4-c]pyridine-2-carbonyl)benzonitrile | | | |
| 27.9 | (2-(Benzyloxy)-4-(difluoromethyl)-6-hydroxyphenyl)(6- | ** | | |
| | morpholino-1,3-dihydro-2H-pyrrolo[3,4-c]pyridin-2- | | | |
| | yl)methanone | | | |
| 27.10 | 4-(6-Amino-2,3-dihydro-1H-pyrrolo[3,4-c]pyridine-2- | * | | |
| | carbonyl)-3-(benzyloxy)-5-hydroxybenzonitrile hydroformate | | | |
| 27.11 | (6-Amino-1,3-dihydro-2H-pyrrolo[3,4-c]pyridin-2-yl)(2- | ** | | |
| | (benzyloxy)-4-(difluoromethyl)-6-hydroxyphenyl)methanone | | | |
| | hydroformate | | | |
| 27.12 | 3-(Benzyloxy)-4-(4-(2-(dimethylamino)ethoxy)indoline-1- | * | | |
| | carbonyl)-5-hydroxybenzonitrile | | | |
| 27.13 | (2-(Benzyloxy)-4-(difluoromethyl)-6-hydroxyphenyl)(4-(2- | ** | | |
| | (dimethylamino)ethoxy)indolin-1-yl)methanone | | | |
| 27.14 | 3-(Benzyloxy)-4-(2,3-dihydro-1H-pyrrolo[2,3-b]pyridine-1- | * | | |
| | carbonyl)-5-hydroxybenzonitrile | | | |
| 27.15 | (2-(Benzyloxy)-4-(difluoromethyl)-6-hydroxyphenyl)(2,3- | * | | |
| | dihydro-1H-pyrrolo[2,3-b]pyridin-1-yl)methanone | | | |
| 27.16 | 3-(Benzyloxy)-4-(2-(2-(dimethylamino)ethoxy)-4- | ** | | |
| | ((tetrahydrofuran-3-yl)amino)-5,6,7,8-tetrahydropyrido[4,3- | | | |
| | d]pyrimidine-6-carbonyl)-5-hydroxybenzonitrile | | | |
| 27.17 | (2-(Benzyloxy)-4-(difluoromethyl)-6-hydroxyphenyl)(2-(2- | ** | | |
| | (dimethylamino)ethoxy)-4-((tetrahydrofuran-3-yl)amino)-7,8- | | | |
| | dihydropyrido[4,3-d]pyrimidin-6(5H)-yl)methanone | | | |
| 27.18 | 3-(Benzyloxy)-5-hydroxy-4-(6-((tetrahydrofuran-3-yl)amino)- | * | | |
| | 1,2,3,4-tetrahydroquinoline-1-carbonyl)benzonitrile | | | |
| 27.19 | (2-(Benzyloxy)-4-(difluoromethyl)-6-hydroxyphenyl)(6- | * | | |
| | ((tetrahydrofuran-3-yl)amino)-3,4-dihydroquinolin-1(2H)- | | | |
| | yl)methanone | | | |
| 27.20 | (2-(Benzyloxy)-4-(difluoromethyl)-6-hydroxyphenyl)(4- | ** | | |
| | ((tetrahydrofuran-3-yl)amino)-7,8-dihydropyrido[4,3- | | | |
| | d]pyrimidin-6(5H)-yl)methanone | | | |
| 27.21 | (2-(Benzyloxy)-4-(difluoromethyl)-6-hydroxyphenyl)(2,3- | * | | |
| | dihydro-1H-pyrrolo[3,2-c]pyridin-1-yl)methanone | | | |
| 27.22 | (2-(Benzyloxy)-4-(difluoromethyl)-6-hydroxyphenyl)(2,3- | * | | |
| | dihydro-1H-pyrrolo[3,2-b]pyridin-1-yl)methanone | | | |
| 27.23 | (2-(Benzyloxy)-4-(difluoromethyl)-6-hydroxyphenyl)(2,3- | * | | |
| | dihydro-1H-pyrrolo[2,3-c]pyridin-1-yl)methanone | | | |
| 27.24 | (2-(Benzyloxy)-4-(difluoromethyl)-6-hydroxyphenyl)(4- | * | | |
| | ((tetrahydrofuran-3-yl)amino)indolin-1-yl)methanone | | | |
| 28 | (S)-(4-(Difluoromethyl)-2-hydroxy-6-((1-methyl-1H-indazol-6- | *** | | |
| | yl)methoxy)phenyl)(8-((tetrahydrofuran-3-yl)amino)-3,4- | | | |
| | dihydroisoquinolin-2(1H)-yl)methanone | | | |
| 28.1 | (S)-(2-((2-Oxabicyclo[2.1.1]hexan-1-yl)methoxy)-4- | * | | |
| | (difluoromethyl)-6-hydroxyphenyl)(8-((tetrahydrofuran-3- | | | |
| | yl)amino)-3,4-dihydroisoquinolin-2(1H)-yl)methanone | | | |
| 28.2 | (S)-(4-(Difluoromethyl)-2-hydroxy-6-(isoquinolin-6- | *** | | |
| | ylmethoxy)phenyl)(8-((tetrahydrofuran-3-yl)amino)-3,4- | | | |
| | dihydroisoquinolin-2(1H)-yl)methanone | | | |
| 28.3 | (S)-(4-(Difluoromethyl)-2-hydroxy-6-(isoquinolin-7- | ** | | |
| | ylmethoxy)phenyl)(8-((tetrahydrofuran-3-yl)amino)-3,4- | | | |
| | dihydroisoquinolin-2(1H)-yl)methanone | | | |
| 28.4 | (S)-(2-((1H-Benzo[d]imidazol-2-yl)methoxy)-4- | * | | |
| | (difluoromethyl)-6-hydroxyphenyl)(8-((tetrahydrofuran-3- | | | |
| | yl)amino)-3,4-dihydroisoquinolin-2(1H)-yl)methanone | | | |
| 29 | 1-(4-((2-(2-(Benzyloxy)-4-(difluoro methyl)-6- | *** | | |
| | hydroxybenzoyl) isoindolin-5-yl) oxy) piperidin-1-yl) ethan-1- | | | |
| | one | | | |

* 1-10 μM
** 0.1-1 μM
*** less than 0.1 μM
PMS2 and HSP90 activity is >10 uM unless otherwise stated.

While specific embodiments of the invention have been described herein for the purpose of reference and illustration, various modifications will be apparent to a person skilled in the art without departing from the scope of the invention as defined by the appended claims.

1. A compound, or a pharmaceutically acceptable salt thereof, having the structural Formula (I) shown below: 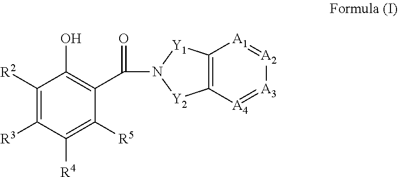
wherein
R² is hydrogen or fluoro;
R³ is selected from halo, cyano, (1-2C)alkyl, (1-2C)haloalkyl, (1-2C)hydroxyalkyl, (1-2C)alkoxy, (1-2C)haloalkoxy;
R⁴ is selected from hydrogen, halo or cyano;
or R³ and R⁴ are linked to form:
a fused 5- or 6-membered carbocyclic or heterocyclic ring;
a fused phenyl ring; or
a 5- or 6-membered heteroaryl ring;
each of which is optionally further substituted by halo, hydroxy, cyano, (1-2C)alkyl, (1-2C)haloalkyl, (1-2C)alkoxy or (1-2C)haloalkoxy;
R⁵ is R⁶ or —OR⁶;
wherein R⁶ is (1-6C)alkyl, (3-8C)cycloalkyl, a 4- to 7-membered heterocyclyl ring comprising one heteroatom selected from N, O or S, phenyl, or a 5- or 6-membered heteroaryl; each of which is optionally substituted by one or more substituents independently selected from the group consisting of hydroxy, cyano, halogen, (1-3C)alkyl, (1-3C)alkoxy, (1-3C)haloalkyl, or (1-3C)haloalkoxy;
or a group having a structure according to formula (A) shown below: 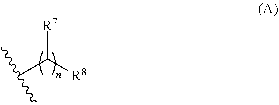
wherein
R⁷ is hydrogen or (1-3C)alkyl;
n is 1 or 2;
R⁸ is (3-8C)cycloalkyl, aryl, heterocyclyl, or heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy;
Y₁ is a bond, —CH₂—, —CH₂—CH₂— or —C(═O)—;
Y₂ is —CH₂—, —C(═O)—, —CHR_{y2a}—, —CH₂—CH₂—, —CH₂—CHR_{y2b}—, or —CHR_{y2a}—CH₂—; where R_{y2a} is selected from halo, cyano, methyl, methoxy, CF₃, —OCF₃ or hydroxymethyl, and R_{y2b} is selected from halo, cyano, hydroxy, methyl, methoxy, CF₃, —OCF₃ or hydroxymethyl, provided that when Y₁ is a bond, Y₂ is —CH₂—CH₂—, —CH₂—CHR_{y2b}—, or —CHR_{y2a}—CH₂—;
A₁ is selected from N, CH or CR¹¹;
A₂ is selected from N, CH or CR¹²;
A₃ is selected from N, CH or CR¹³;
A₄ is selected from N, CH or CR¹⁴;
with the proviso that only one or two of A₁, A₂, A₃ or A₄ can be N;
R¹¹ is cyano, halo, (1-2C)alkyl, (1-2C)alkoxy, wherein any (1-2C)alkyl or (1-2C)alkoxy is optionally substituted by one or more halo or (1-2C)alkoxy;
or R¹¹ is —(CHR_{z})ₐ—Z¹¹,
wherein:
R_{z} is hydrogen or methyl;
a is 0 or 1; and
Z¹¹ is —OR¹⁵, R^{15a}, —NR¹⁶R¹⁷, —C(O)NR¹⁶R¹⁷ or —NR¹⁸C(O)R¹⁹;
wherein
R¹⁵ is (1-4C)alkyl, (3-7C)cycloalkyl, phenyl, a carbon-linked 4 to 8-membered heterocyclyl, a 5 or 6 membered heteroaryl, —(CHR_{y})_{b}-(3-7C)cycloalkyl, —(CHR_{y})_{b}-phenyl, —(CHR_{y})_{b}-[4 to 8-membered heterocyclyl] or —(CHR_{y})_{b}-[5 or 6 membered heteroaryl], wherein R_{y} is hydrogen or methyl and b is 0, 1 or 2;
R^{15a} is (1-4C)alkyl, (3-7C)cycloalkyl, phenyl, 4 to 8-membered heterocyclyl, a 5 or 6 membered heteroaryl, —(CHR_{y})_{b}-(3-7C)cycloalkyl, —(CHR_{y})_{b}-phenyl, —(CHR_{y})_{b}-[4 to 8-membered heterocyclyl] or —(CHR_{y})_{b}-[5 or 6 membered heteroaryl], wherein R_{y} is hydrogen or methyl and b is 0, 1 or 2;
R¹⁶ and R¹⁷ are each independently selected from hydrogen, (1-6C)alkyl, (3-7C)cycloalkyl, phenyl, a carbon-linked 4 to 8-membered heterocyclyl, a 5 or 6 membered heteroaryl, —(CHR_{w})_{c}-(3-7C)cycloalkyl, —(CHR_{w})_{c}-phenyl, —(CHR_{w})_{c}-[4 to 8-membered heterocyclyl] or —(CHR_{w})_{c}-[5 or 6 membered heteroaryl], wherein R_{y} is hydrogen or methyl and c is 0, 1 or 2;
or R¹⁶ and R¹⁷ are linked, such that, together with the nitrogen atom to which they are attached, they form a 4-8 membered heterocyclic ring;
R¹⁸ is hydrogen or (1-2C)alkyl;
R¹⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₓ)_{d}-(3-7C)cycloalkyl, —(CHRₓ)_{d}-phenyl, —(CHRₓ)_{d}-[4 to 8-membered heterocyclyl] or —(CHRₓ)_{d}-[5 or 6 membered heteroaryl];
wherein R is hydrogen or methyl and d is 0 or 1;
wherein each of R¹¹, R¹⁵, R^{15a}, R¹⁶, R¹⁷, R¹⁸ or R¹⁹, or any ring formed when R¹⁶ and R¹⁷ are linked, is optionally substituted with one or more R^{a};
R¹² is selected from one of the following options:
(i) when R¹¹ is a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
(ii) when R¹¹ is not a group —(CHR_{z})ₐ—Z¹¹ then R¹² is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹² is —(CHRᵥ)_{d}—Z¹²,
wherein:
Rᵥ is hydrogen or methyl;
d is 0 or 1; and
Z¹² is —OR²⁰, R^{20a}, —NR²¹R²², —C(O)NR²¹R²² or —NR²³C(O)R²⁴;
wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rᵤ is hydrogen or methyl and e is 0, 1, 2 or 3;
R^{20a} is (1-4C)alkyl, (3-7C)cycloalkyl, phenyl, 4 to 8-membered heterocyclyl, a 5 or 6 membered heteroaryl, —(CHRᵤ)ₑ-(3-7C)cycloalkyl, (CHRᵤ)ₑ-phenyl, —(CHRᵤ)ₑ-[4 to 8-membered heterocyclyl] or —(CHRᵤ)ₑ-[5 or 6 membered heteroaryl], wherein Rᵤ is hydrogen or methyl and e is 0, 1, 2 or 3;
R²¹ and R²² are each independently selected from hydrogen, (1-6C)alkyl, (2-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[4 to 8-membered heterocyclyl] or —(CHR_{q})_{f}-[5 or 6 membered heteroaryl], wherein R_{q} is hydrogen or methyl and f is 0 or 1;
or R²¹ and R²² are linked, such that, together with the nitrogen atom to which they are attached, they form a 4-8 membered heterocyclic ring;
R²³ is hydrogen or (1-2C)alkyl;
R²⁴ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
wherein each of R¹², R²⁰, R^{20a}, R²¹, R²², R²³ or R²⁴, or any ring formed when R²¹ and R²² are linked, is optionally substituted with one or more R^{a};
R¹³ is selected from cyano, halo, (1-2C)alkyl, (1-2C)alkoxy, wherein any (1-2C)alkyl moiety is optionally substituted by one or more halo or (1-2C)alkoxy, or
R¹³ is —(CHRₒ)ₕ—Z¹³,
wherein:
Rₒ is hydrogen or methyl;
h is 0 or 1; and
Z¹³ is —OR²⁵, R^{25a}, —NR²⁶R²⁷, —C(O)NR²⁶R²⁷ or —NR²⁸C(O)R²⁹;
wherein R²⁵ is (1-4C)alkyl, (3-7C)cycloalkyl, phenyl, 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ—[4 to 8-membered heterocyclyl] or —(CHRₙ)ᵢ-[5 or 6 membered heteroaryl], wherein Rₙ is hydrogen or methyl and i is 0, 1 or 2;
R^{25a} is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ—[5 or 6 membered heteroaryl], wherein Rₙ and i are as defined above;
R²⁶ and R²⁷ are each independently selected from hydrogen, (1-6C)alkyl, (2-6C)alkanoyl, (3-7C)cycloalkyl, phenyl a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
or R²⁶ and R²⁷ are linked, such that, together with the nitrogen atom to which they are attached, they form a 4-8 membered heterocyclic ring;
R²⁸ is hydrogen or (1-2C)alkyl;
R²⁹ is (1-6C)alkyl, (2-6C)alkynyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
wherein each of R¹³, R²⁵, R^{25a}, R²⁶, R²⁷, R²⁸ or R²⁹ or any ring formed when R²⁶ and R²⁷ are linked, is optionally substituted with one or more R^{a};
R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹⁴ is —(CHRₖ)ₘ—Z¹⁴,
wherein Rₖ is hydrogen or methyl;
wherein m is 0 or 1; and
Z¹⁴ is —OR³⁰, R^{30a}, —NR³¹R³², —C(O)NR³¹R³² or —NR³³C(O)R³⁴;
wherein R³⁰ is (1-4C)alkyl, (3-7C)cycloalkyl, a carbon-linked 4 to 8-membered heterocyclyl, a 5 or 6 membered heteroaryl, —(CHRⱼ)ₒ-(3-7C)cycloalkyl, —(CHRⱼ)ₒ-[4 to 8-membered heterocyclyl] or —(CHRⱼ)ₒ-[5 or 6 membered heteroaryl], wherein R; is hydrogen or methyl and o is 0 or 1;
R^{30a} is (1-4C)alkyl, (3-7C)cycloalkyl, 4 to 8-membered heterocyclyl, a 5 or 6 membered heteroaryl, —(CHRⱼ)ₒ-(3-7C)cycloalkyl, —(CHRⱼ)ₒ-[4 to 8-membered heterocyclyl] or —(CHRⱼ)ₒ-[5 or 6 membered heteroaryl], wherein Rⱼ is hydrogen or methyl and o is 0 or 1;
R³¹ and R³² are each independently selected from hydrogen, (1-6C)alkyl, (2-6C)alkanoyl, (3-7C)cycloalkyl, a carbon-linked 4 to 8-membered heterocyclyl, a 5 or 6 membered heteroaryl, —(CHRⱼ)ₚ-(3-7C)cycloalkyl, —(CHRⱼ)ₚ-[4 to 8-membered heterocyclyl] or —(CHRⱼ)ₚ-[5 or 6 membered heteroaryl], wherein R; is hydrogen or methyl and p is 0 or 1
or R³¹ and R³² are linked, such that, together with the nitrogen atom to which they are attached, they form a 4-8 membered heterocyclic ring;
R³³ is hydrogen or (1-2C)alkyl;
R³⁴ is (1-6C)alkyl, (2-6C)alkynyl, (3-7C)cycloalkyl, a carbon-linked 4 to 8-membered heterocyclyl, a 5 or 6 membered heteroaryl, —(CHRₕ)_{q}-(3-7C)cycloalkyl, —(CHRₕ)_{q}-[4 to 8-membered heterocyclyl] or —(CHRₕ)_{q}-[5 or 6 membered heteroaryl], wherein Rₕ is hydrogen or methyl and q is 0 or 1;
wherein R¹⁴, R³⁰, R^{30a}, R³¹, R³², R³³ or R³⁴, or any ring formed when R³¹ and R³² are linked, is optionally substituted with one or more R^{a};
and wherein each R^{a} is independently selected from the group consisting of oxo, halogen, cyano, hydroxy, (1-4C)alkyl, or a group 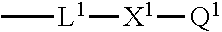
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —N(R¹⁴)—C(O)—NR¹⁴—, —SO₂N(R¹⁴)—, or —N(R¹⁴)SO₂—, where R¹⁴ is hydrogen or (1-2C)alkyl;
Q¹ is selected from the group consisting of hydrogen, (1-4C)alkyl, (2-4C)alkenyl, (2-4C)alkynyl, or (3-6C)cycloalkyl;
wherein when Q¹ is (1-4C)alkyl, (2-4C)alkenyl, (2-4C)alkynyl, or (3-6C)cycloalkyl, Q¹ is optionally substituted with one or more Q^{a}, where each Q^{a} is independently selected from the group consisting of hydroxy, cyano, halogen, (1-3C)alkyl, (1-3C)alkoxy, (1-3C)haloalkyl, or (1-3C)haloalkoxy;
and wherein one of R¹² or R¹³ is optionally a group: 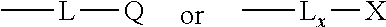
wherein L is a linker;
Lₓ is a linker;
X is a functional group; and
Q is a detection moiety.

2. A compound according to claim 1 or pharmaceutically acceptable salt, hydrate or solvate thereof, wherein R² is hydrogen.

3. A compound according to claim 1 or claim 2, wherein the compound is a compound having one of the formulae (I-I) to (I-XLV) shown below, or pharmaceutically acceptable salt thereof: 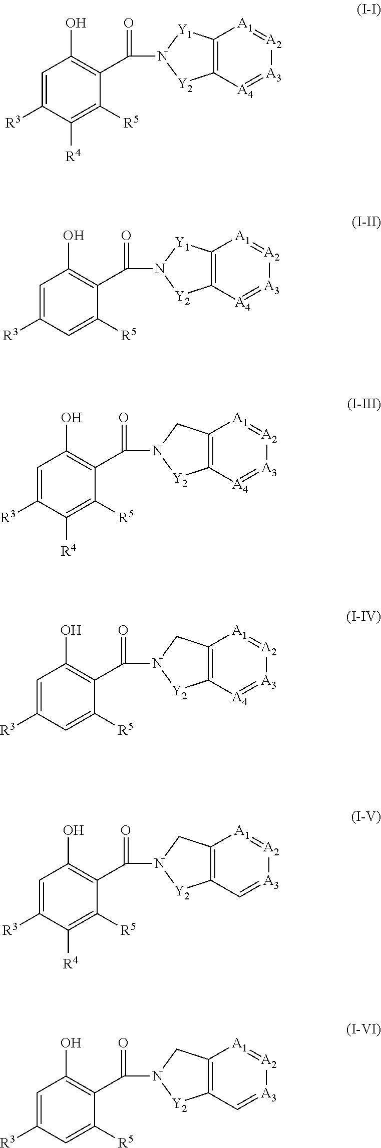 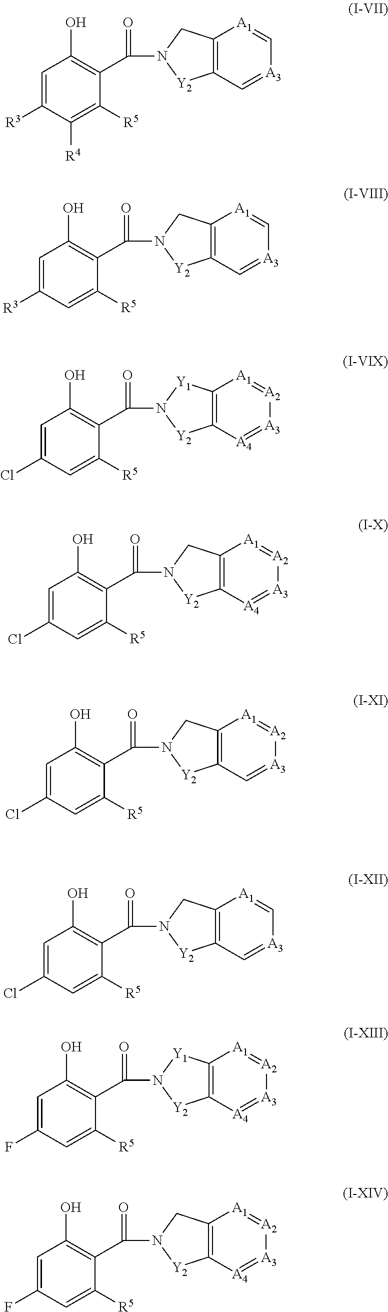 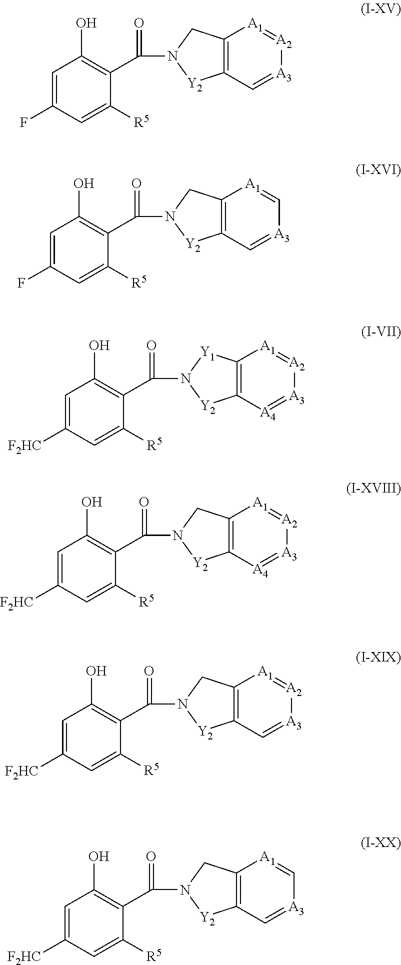 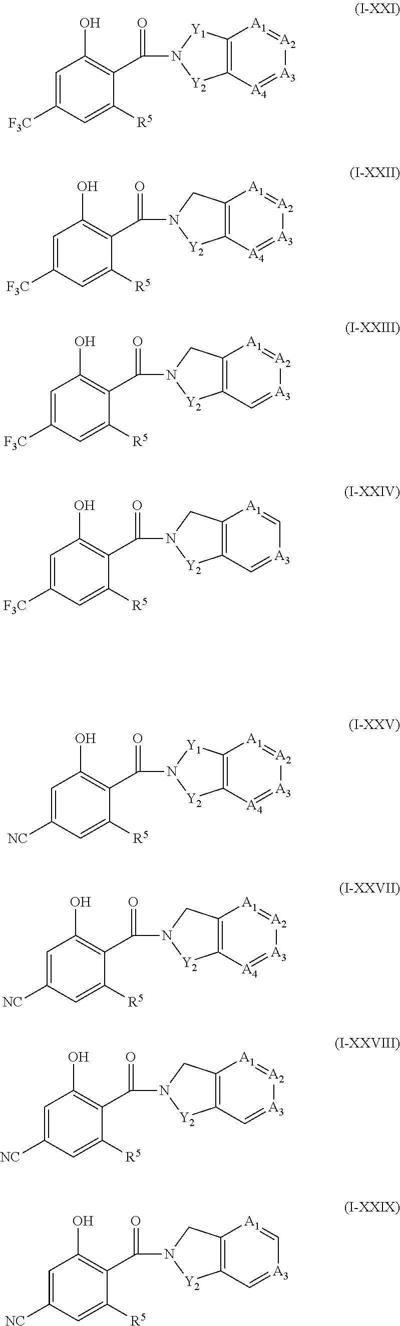 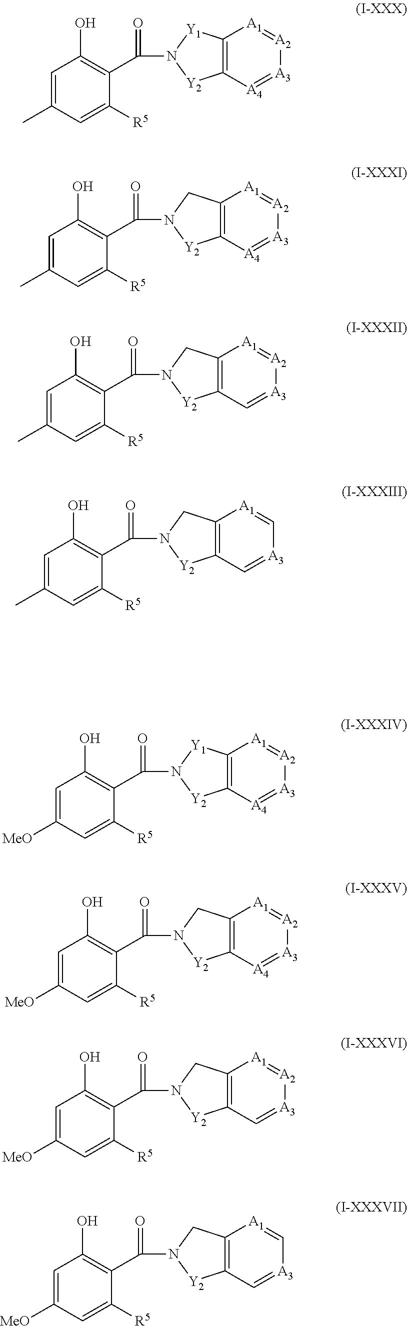 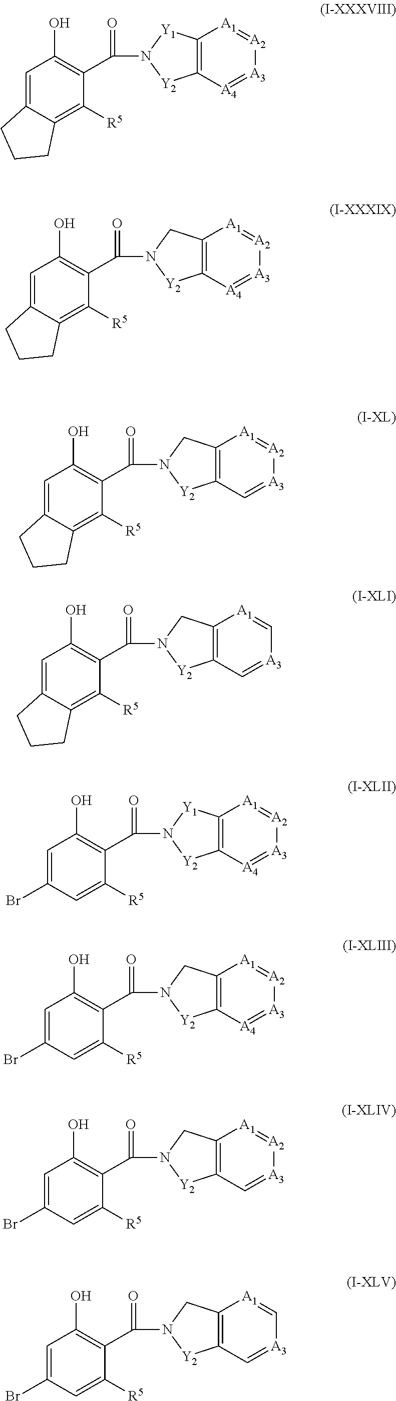
wherein R³, R⁴, R⁵, Y₁, Y₂, A₁, A₂, A₃ and A₄ are each as defined in claim 1.

4. A compound according to any one of claims 1 to 3, or pharmaceutically acceptable salt thereof, wherein:
R⁴ is hydrogen and R³ is selected from halo, cyano, (1-2C)alkyl, (1-2C)haloalkyl, (1-2C)hydroxyalkyl, (1-2C)alkoxy, (1-2C)haloalkoxy;
or R³ and R⁴ are linked to form a fused 5- or 6-membered carbocyclic or heterocyclic ring, which is optionally further substituted by halo, hydroxy, cyano, (1-2C)alkyl, (1-2C)haloalkyl, (1-2C)alkoxy or (1-2C)haloalkoxy.

5. A compound according to any one of claims 1 to 4, or pharmaceutically acceptable salt thereof, wherein:
R⁴ is hydrogen and R³ is selected from halo, cyano, (1-2C)alkyl, (1-2C)fluoroalkyl, (1-2C)alkoxy, (1-2C)fluoroalkoxy;
or R³ and R⁴ are linked to form a fused 5- or 6-membered carbocyclic or heterocyclic ring, which is optionally further substituted by halo, hydroxy, cyano, (1-2C)alkyl, (1-2C)fluoroalkyl, (1-2C)alkoxy or (1-2C)fluoroalkoxy.

6. A compound according to any one of claims 1 to 5, or pharmaceutically acceptable salt thereof, wherein:
R⁴ is hydrogen and R³ is selected from halo, cyano, methyl, fluoromethyl, di-fluoromethyl, trifluoromethyl, methoxy, di-fluoromethoxy or trifluoromethoxy;
or R³ and R⁴ are linked to form a fused 5- or 6-membered carbocyclic or heterocyclic ring, which is optionally further substituted by halo, cyano, methyl, fluoromethyl, di-fluoromethyl, trifluoromethyl, methoxy, di-fluoromethoxy or trifluoromethoxy.

7. A compound according to any one of claims 1 to 6, or pharmaceutically acceptable salt thereof, wherein:
R⁴ is hydrogen and R³ is selected from halo, cyano, methyl, di-fluoromethyl, trifluoromethyl, or methoxy;
or R³ and R⁴ are linked to form a fused 5- or 6-membered carbocyclic or heterocyclic ring, which is optionally further substituted by halo, cyano, methyl, di-fluoromethyl, trifluoromethyl or methoxy.

8. A compound according to any one of claims 1 to 7, or pharmaceutically acceptable salt thereof, wherein:
R⁴ is hydrogen and R³ is selected from halo, cyano, methyl, di-fluoromethyl, trifluoromethyl, or methoxy;
or R³ and R⁴ are linked to form a fused 5- or 6-membered carbocyclic or heterocyclic ring.

9. A compound according to any one of claims 1 to 8, or pharmaceutically acceptable salt thereof, wherein:
R⁴ is hydrogen and R³ is selected from halo, cyano, methyl, di-fluoromethyl, trifluoromethyl, or methoxy;
or R³ and R⁴ are linked to form a fused 5- or 6-membered cycloalkyl ring.

10. A compound according to any one of claims 1 to 9, or pharmaceutically acceptable salt thereof, wherein R⁵ is —OR⁶.

11. A compound according to any one of claims 1 to 10, or pharmaceutically acceptable salt thereof, wherein R⁵ is —OR⁶, wherein:
(i) R⁶ is (1-6C)alkyl, (3-6C)cycloalkyl, or a 4- to 6-membered heterocyclyl ring comprising one heteroatom selected from N, O or S,
or a group having a structure according to formula (A) shown below: 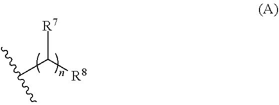
wherein
R⁷ is hydrogen or (1-3C)alkyl;
n is 1 or 2;
R⁸ is (3-8C)cycloalkyl, phenyl, 4- to 6-membered heterocyclyl, 5- or 6-membered heteroaryl, or 9- or 10-membered fused bicyclic heteroaryl, each of which is optionally substituted with one or more R⁹; where each R⁹ is independently selected from the group consisting of hydroxy, cyano, halogen, (1-3C)alkyl, (1-3C)alkoxy, (1-3C)haloalkyl, or (1-3C)haloalkoxy, and wherein any 4- to 6-membered heterocyclyl optionally comprises 1 or 2 additional bridging atoms;
(ii) R⁶ is (1-4C)alkyl or a group having a structure according to formula (A) shown below: 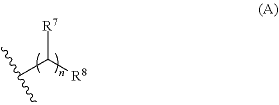
wherein
R⁷ is hydrogen or (1-2C)alkyl;
n is 1 or 2;
R⁸ is (3-6C)cycloalkyl, phenyl, 4- to 6-membered heterocyclyl, 5- or 6-membered heteroaryl, or 9- or 10-membered fused bicyclic heteroaryl, each of which is optionally substituted with one or more R⁹; where each R⁹ is independently selected from the group consisting of hydroxy, cyano, halogen, (1-2C)alkyl, (1-2C)alkoxy, (1-2C)haloalkyl, or (1-2C)haloalkoxy, and wherein any 4- to 6-membered heterocyclyl optionally comprises 1 additional bridging atom;
(iii) R⁶ is (1-3C)alkyl or a group having a structure according to formula (A) shown below: 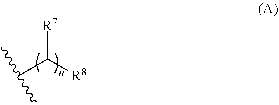
wherein
R⁷ is hydrogen or methyl;
n is 1;
R⁸ is (3-6C)cycloalkyl, phenyl, 6-membered heterocyclyl, 6-membered heteroaryl, or 9- or 10-membered fused bicyclic heteroaryl, each of which is optionally substituted with one or more R⁹; where each R⁹ is independently selected from the group consisting of halogen, methyl, methoxy, trifluoromethyl, or trifluoromethoxy;
(iv) R⁶ is methyl, ethyl or a group having a structure according to formula (A) shown below: 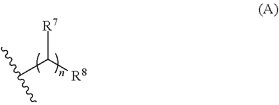
wherein
R⁷ is hydrogen or methyl;
n is 1;
R⁸ is cyclopropyl, cyclohexyl, phenyl, 6-membered heterocyclyl, pyridyl, benzimidazole or quinoline, each of which is optionally substituted with one or more R⁹; where each R⁹ is independently selected from the group consisting of halogen, methyl, methoxy, trifluoromethyl, or trifluoromethoxy; or
(v) R⁶ is methyl or a group having a structure according to formula (A) shown below: 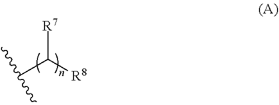
wherein
R⁷ is hydrogen;
n is 1;
R⁸ is cyclopropyl or phenyl.
(vi) R⁶ is a group having a structure according to formula (A) shown below: 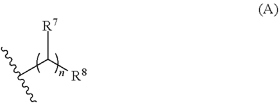
wherein
R⁷ is hydrogen;
n is 1;
R⁸ is phenyl.

12. A compound according to any one of claims 1 to 11, or pharmaceutically acceptable salt, hydrate or solvate thereof, wherein:
(i) Y₁ is a bond, —CH₂— or —CH₂—CH₂—; and/or
(ii) Y₂ is —CH₂—, —CHR_{y2a}—, —CH₂—CH₂—, —CH₂—CHR_{y2b}—, or —CHR_{y2a}—CH₂—; where R_{y2a} is selected from halo, methyl or hydroxymethyl, and R_{y2b} is selected from halo, cyano, hydroxy, methyl, methoxy, CF₃, —OCF₃ or hydroxymethyl, provided that when Y₁ is a bond, Y₂ is —CH₂—CH₂—, —CH₂—CHR_{y2b}—, or —CHR_{y2a}—CH₂—; or
(iii) Y₂ is —CH₂— or —CH₂—CH₂—, provided that when Y₁ is a bond, Y₂ is —CH₂—CH₂—.

13. A compound according to any one of claims 1 to 12, or pharmaceutically acceptable salt, hydrate or solvate thereof, wherein A₁, A₂, A₃ or A₄ are selected from one of the following options:
(i) A₁ is selected from N, CH or OR¹¹;
A₂ is selected from N, CH or CR¹²;
A₃ is selected from N, CH or CR¹³;
A₄ is selected from N, CH or CR¹⁴;
with the proviso that only one of A₁, A₂, A₃ or A₄ can be N.
(ii) A₁ is selected from CH or OR¹¹;
A₂ is selected from N, CH or CR¹²;
A₃ is selected from N, CH or CR¹³;
A₄ is selected from N, CH or CR¹⁴;
with the proviso that only one of A₂, A₃ or A₄ can be N;
(iii) A₁ is CR¹¹;
A₂ is selected from N, CH or CR¹²;
A₃ is selected from N, CH or CR¹³;
A₄ is selected from N, CH or CR¹⁴;
with the proviso that only one of A₂, A₃ or A₄ can be N;
(iv) A₁ is OR¹¹;
A₂ is selected from N or CH;
A₃ is selected from N, CH or CR¹³;
A₄ is selected from N or CH;
with the proviso that only one of A₂, A₃ or A₄ can be N;
(v) A₁ is CR¹¹;
A₂ is CH;
A₃ is CH or CR¹³;
A₄ is CH;
(vi) A₁ is CR¹¹;
A₂ is CH;
A₃ is CH;
A₄ is CH;
(vii) A₁ is CH;
A₂ is CR¹²;
A₃ is CH;
A₄ is CH; or
(viii) A₁ is CH;
A₂ is CH;
A₃ is CR¹³;
A₄ is CH.

14. A compound according to any one of claims 1 to 11, or pharmaceutically acceptable salt thereof, wherein A₁, A₂, A₃ or A₄ are selected from one of the following options:
(i) A₁ is CH or CR¹¹;
A₂ is CH;
A₃ is CH or CR¹³;
A₄ is CH;
(ii) A₁ is CR¹¹;
A₂ is CH;
A₃ is CH;
A₄ is CH;
(iii) A₁ is CH;
A₂ is CR¹²;
A₃ is CH;
A₄ is CH; or
(iv) A₁ is CH;
A₂ is CH;
A₃ is CR¹³;
A₄ is CH.

15. A compound according to any one of claims 1 to 11, or pharmaceutically acceptable salt thereof, wherein
A₁ is CH or CR¹¹;
A₂ is CH;
A₃ is CH or CR¹³; and
A₄ is CH.

16. A compound according to any one of claims 1 to 15, or pharmaceutically acceptable salt, hydrate or solvate thereof, wherein:
(A) R¹¹ is cyano, halo or (1-2C)alkyl optionally substituted by one or more R^{a};
or —(CHR_{z})ₐ—Z¹¹,
wherein R_{z} is hydrogen or methyl;
wherein a is 0 or 1; and
Z¹¹ is —OR¹⁵, —NR¹⁶R¹⁷, —C(O)NR¹⁶R¹⁷ or —NR¹⁸C(O)R¹⁹;
wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
or R¹⁶ and R¹⁷ are linked, such that, together with the nitrogen atom to which they are attached, they form a 4-6 membered heterocyclic ring;
R¹⁸ is hydrogen or (1-2C)alkyl;
R¹⁹ is (1-6C)alkyl, (3-6C)cycloalkyl, phenyl, a carbon-linked 4 to 6-membered heterocyclyl, a 5 or 6 membered heteroaryl, —(CHRₓ)_{d}-(3-6C)cycloalkyl, —(CHRₓ)_{d}-phenyl, —(CHRₓ)_{d}-[4 to 6-membered heterocyclyl] or —(CHRₓ)_{d}-[5 or 6 membered heteroaryl];
wherein Rₓ is hydrogen or methyl and d is 0 or 1;
wherein each of R¹⁵, R¹⁶, R¹⁷, R¹⁸ or R¹⁹, or any ring formed when R¹⁶ and R¹⁷ are linked, is optionally substituted with one or more R^{a};
wherein R^{a} is as defined in claim 1;
(B) R¹¹ is cyano or —(CHR_{z})ₐ—Z¹¹,
wherein R_{z} is hydrogen or methyl;
wherein a is 0 or 1; and
Z¹¹ is —OR¹⁵, —NR¹⁶R¹⁷ or —NR¹⁸C(O)R¹⁹;
wherein R¹⁵ is (1-4C)alkyl, (3-6C)cycloalkyl, phenyl, a carbon-linked 4 to 6-membered heterocyclyl, a 5 or 6 membered heteroaryl, —(CHR_{y})_{b}-(3-6C)cycloalkyl, —(CHR_{y})_{b}-phenyl, —(CHR_{y})_{b}-[4 to 6-membered heterocyclyl] or —(CHR_{y})_{b}-[5 or 6 membered heteroaryl], wherein R_{y} is hydrogen or methyl and b is 0 or 1;
R¹⁶ and R¹⁷ are each independently selected from hydrogen, (1-6C)alkyl, (3-6C)cycloalkyl, phenyl, a carbon-linked 4 to 6-membered heterocyclyl, a 5 or 6 membered heteroaryl, —(CHR_{w})_{c}-(3-6C)cycloalkyl, —(CHR_{w})_{c}-phenyl, —(CHR_{w})_{c}-[4 to 6-membered heterocyclyl] or —(CHR_{w})_{c}-[5 or 6 membered heteroaryl], wherein R_{w} is hydrogen or methyl and c is 0 or 1
or R¹⁶ and R¹⁷ are linked, such that, together with the nitrogen atom to which they are attached, they form a 4-6 membered heterocyclic ring;
R¹⁸ is hydrogen or (1-2C)alkyl;
R¹⁹ is (1-6C)alkyl, (3-6C)cycloalkyl, phenyl, a carbon-linked 4 to 6-membered heterocyclyl, a 5 or 6 membered heteroaryl, or —(CHRₓ)_{d}-(3-6C)cycloalkyl;
wherein Rₓ is hydrogen or methyl and d is 0 or 1;
wherein each of R¹⁵, R¹⁶, R¹⁷, R¹⁸ or R¹⁹, or any ring formed when R¹⁶ and R¹⁷ are linked, is optionally substituted with one or more R^{a};
wherein R^{a} is as defined in claim 1;
(C) R¹¹ is —(CHR_{z})ₐ—Z¹¹,
wherein R_{z} is hydrogen;
wherein a is 0 or 1; and
Z¹¹ is: —OR¹⁵, wherein R¹⁵ is (1-4C)alkyl; or
—NHR¹⁷, wherein R¹⁷ is selected from:
(i) a group of the formula (A), (B), (C) or (D): 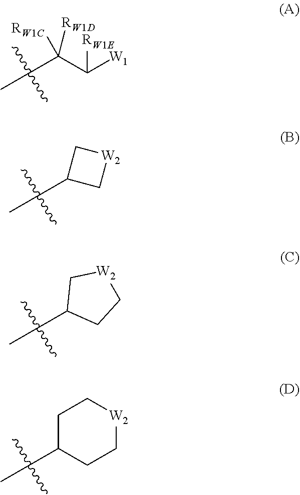
wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen or methyl, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2}, S or S(O)₂ where R_{w2} is selected from hydrogen, (1-4C)alkyl or (2-4C)alkanoyl;
and each of formulae (B), (C) and (D) is optionally substituted by one or more R^{a};
wherein R^{a} is as defined in claim 1;
(ii) a bridged version of formulae (C) and (D) above, optionally selected from one of formulae (E) to (J) below: 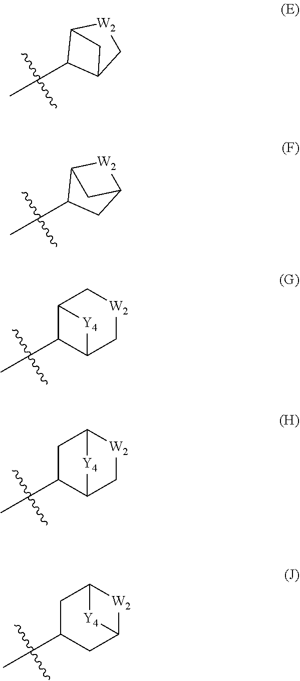
wherein Y₄ is selected from —CH₂—, —CH₂CH₂—,
and each of formulae (E), (F), (G), (H) and (J) is optionally substituted by one or more R^{a};
wherein R^{a} is as defined in claim 1;
(iii) a group of the formula (K), (L) or (M) below: 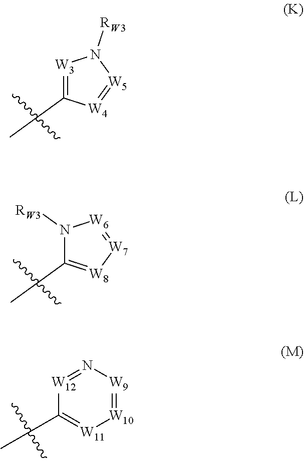
wherein:
R_{W3} is selected from hydrogen or (1-2C)alkyl;
W₃, W₄ and W₅ are selected from N or CR_{w4} where R_{w4} is hydrogen, halo, (1-2C)alkyl or (1-2C)alkoxy;
one of W₆ and W₇ is N and the other is CR_{w5} and W₈ is N or CR_{w5}, where R_{w5} is hydrogen, halo, (1-2C)alkyl or (1-2C)alkoxy;
W₉, W₁₀, W₁₁ and W₁₂ are selected from N or CR_{w6} where R_{w6} is hydrogen, halo, (1-2C)alkyl or (1-2C)alkoxy; with the proviso that only one or two of W₉, W₁₀, W₁₁ and W₁₂ are N;
(D) R¹¹ is —(CHR_{z})ₐ—Z¹¹,
wherein R_{z} is hydrogen;
wherein a is 0 or 1; and
Z¹¹ is: —OR¹⁵, wherein R¹⁵ is (1-3C)alkyl; or
—NHR¹¹, wherein R¹⁷ is selected from:
(i) a group of the formula (A), (B), (C) or (D): 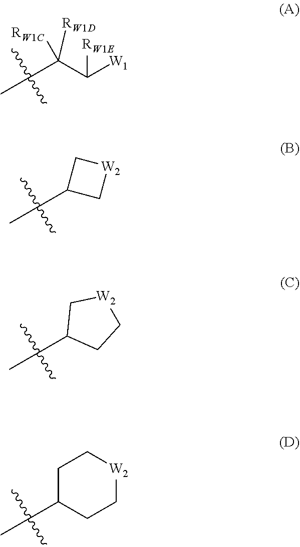
wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from 0, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined in claim 1;
(ii) a bridged version of formulae (C) and (D) above, optionally selected from one of formulae (E) to (J) below: 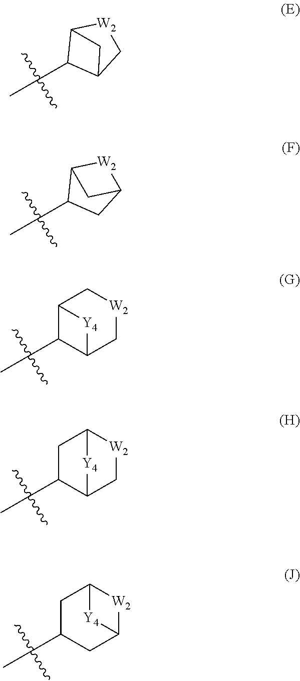
wherein Y₄ is selected from —CH₂—, —CH₂CH₂—,
and each of formulae (E), (F), (G), (H) and (J) is optionally substituted by one or more R^{a};
wherein R^{a} is as defined in claim 1;
(iii) a group of the formula (K), (L) or (M) below: 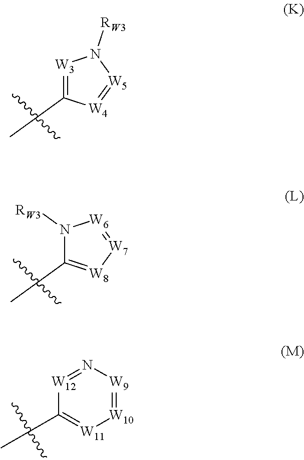
wherein:
R_{W3} is selected from hydrogen or (1-2C)alkyl;
W₃, W₄ and W₅ are selected from N or CH;
one of W₆ and W₇ is N and the other is CH and W₈ is N or CH;
W₉, W₁₀, W₁₁ and W₁₂ are selected from N or CR_{w6} where R_{w6} is hydrogen, halo, (1-2C)alkyl or (1-2C)alkoxy; with the proviso that only one of W₉, W₁₀, W₁₁ and W₁₂ is N.
(E) R¹¹ is —(CHR_{z})ₐ—Z¹¹,
wherein R_{z} is hydrogen;
wherein a is 0 or 1; and
Z¹¹ is: —OR¹⁵, wherein R¹⁵ is (1-2C)alkyl; or
—NHR¹⁷, wherein R¹⁷ is a group of the formula (A), (B), (C) or (D): 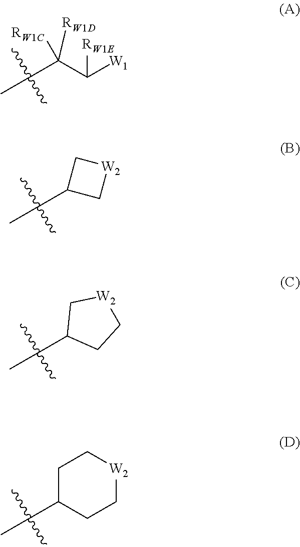
wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined in claim 1; or
a group of the formula (K), (L) or (M) below: 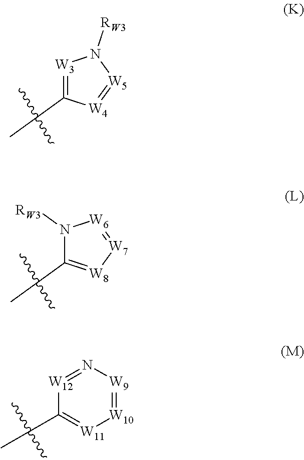
wherein:
R_{W3} is selected from hydrogen or methyl;
W₃, W₄ and W₅ are selected from N or CH;
one of W₆ and W₇ is N and the other is CH and W₈ is N or CH;
W₉, W₁₀, W₁₁ and W₁₂ are selected from N or CR_{w6} where R_{w6} is hydrogen, halo, (1-2C)alkyl or (1-2C)alkoxy; with the proviso that only one of W₉, W₁₀, W₁₁ and W₁₂ is N; or
(F) R¹¹ is Z¹¹,
Z¹¹ is: —OR¹⁵, wherein R¹⁵ is (1-2C)alkyl; or
—NHR¹⁷, wherein R¹⁷ is selected from:
a group of the formula (A), (B), (C), (D) or (M): 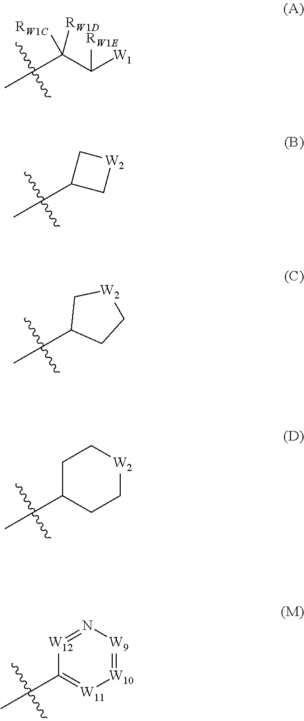
wherein
W₁ is selected from OR_{w1a} or NR_{w1a}R_{w1b}, where R_{w1a} and R_{w1b} are each independently selected from hydrogen or (1-2C)alkyl;
R_{W1C}, R_{W1D} and R_{W1E} are selected from hydrogen, or R_{W1C} and R_{W1D} or R_{W1D} and R_{W1E} are linked such that, together with the carbon atoms to which they are attached, they form a (4-6C)cycloalkyl ring or 5 or 6 membered heterocyclyl ring, each of which is optionally substituted by one or more R^{a};
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by one or more R^{a};
wherein R^{a} is as defined in claim 1;
W₉, W₁₀, W₁₁ and W₁₂ are selected from N or CR_{w6} where R_{w6} is hydrogen, halo, (1-2C)alkyl or (1-2C)alkoxy; with the proviso that only one of W₉, W₁₀, W₁₁ and W₁₂ is N;
(G) R¹¹ is Z¹¹,
Z¹¹ is: —OR¹⁵, wherein R¹⁵ is methyl; or
—NHR¹⁷, wherein R¹⁷ is selected from:
a group of the formula (B), (C), (D) or (M): 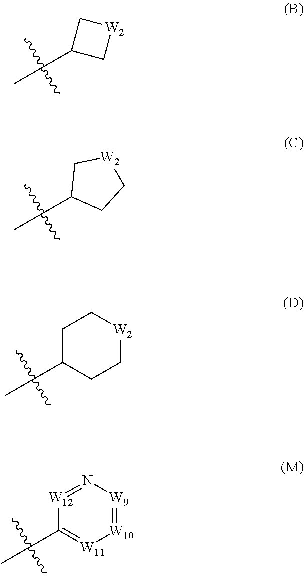
wherein
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by oxo (═O);
W₉, W₁₀, W₁₁ and W₁₂ are selected from N or CH; with the proviso that only one of W₉, W₁₀, W₁₁ and W₁₂ is N.
(H) R¹¹ is Z¹¹,
Z¹¹ is: —OR¹⁵, wherein R¹⁵ is methyl; or
—NHR¹⁷, wherein R¹⁷ is selected from:
a group of the formula (B), (C) or (D): 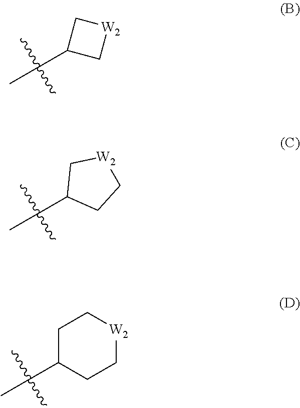
wherein
W₂ is selected from O, NR_{w2} or S(O)₂ where R_{w2} is selected from hydrogen, (1-2C)alkyl or (2C)alkanoyl;
and each of formulae (B), (C) and (D) is optionally substituted by oxo (═O).
(I) R¹¹ is Z¹¹,
Z¹¹ is —NHR¹⁷, wherein R¹⁷ is selected from:
a group of the formula (B), (C) or (D): 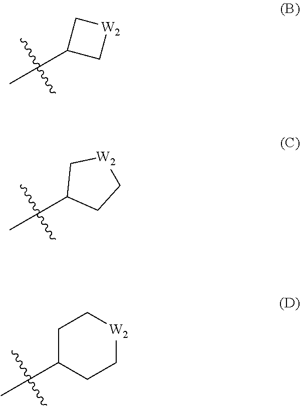
wherein
W₂ is O;
and each of formulae (B), (C) and (D) is optionally substituted by oxo (═O);
(J) R¹¹ is Z¹¹,
Z¹¹ is —NHR¹⁷, wherein R¹⁷ is selected from:
a group of the formula (B): 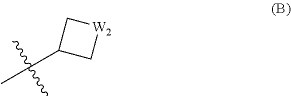
wherein
W₂ is O.

17. A compound according to any one of claims 1 to 16, or pharmaceutically acceptable salt, hydrate or solvate thereof, wherein:
(A) R¹² is selected from one of the following options:
(i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
(ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
R¹² is —(CHRᵥ)_{d}—Z¹²
wherein Rᵥ is hydrogen or methyl;
wherein d is 0 or 1; and
Z¹² is —OR²⁰, —NR²¹R²², —C(O)NR²¹R²² or —NR²¹C(O)R²⁴;
wherein R²⁰ is (1-4C)alkyl, (3-7C)cycloalkyl, phenyl, a carbon-linked 4 to 8-membered heterocyclyl, a 5 or 6 membered heteroaryl, —(CHRᵤ)ₑ-(3-7C)cycloalkyl, —(CHRᵤ)ₑ-phenyl, —(CHRᵤ)ₑ-[4 to 8-membered heterocyclyl] or —(CHRᵤ)ₑ-[5 or 6 membered heteroaryl], wherein Rᵤ is hydrogen and e is 0, 1 or 2;
R²¹ and R²² are each independently selected from hydrogen, (1-6C)alkyl, (3-6C)alkanoyl, (3-7C)cycloalkyl, phenyl, a carbon-linked 4 to 8-membered heterocyclyl, a 5 or 6 membered heteroaryl, —(CHR_{q})_{f}-(3-7C)cycloalkyl, —(CHR_{q})_{f}-phenyl, —(CHR_{q})_{f}-[4 to 8-membered heterocyclyl] or —(CHR_{q})_{f}-[5 or 6 membered heteroaryl], wherein R_{q} is hydrogen or methyl and f is 0 or 1;
or R²¹ and R²² are linked, such that, together with the nitrogen atom to which they are attached, they form a 4-8 membered heterocyclic ring;
R²³ is hydrogen or methyl;
R²⁴ is (1-6C)alkyl, (3-7C)cycloalkyl, phenyl, a carbon-linked 4 to 8-membered heterocyclyl, a 5 or 6 membered heteroaryl, —(CHRₚ)_{g}-(3-7C)cycloalkyl, —(CHRₚ)_{g}-phenyl, —(CHRₚ)_{g}-[4 to 8-membered heterocyclyl] or —(CHRₚ)_{g}-[5 or 6 membered heteroaryl], wherein Rₚ is hydrogen or methyl and g is 0 or 1;
wherein each of R²⁰, R²¹, R²², R²³ or R²⁴, or any ring formed when R²¹ and R²² are linked, is optionally substituted with one or more R^{a};
wherein R^{a} is as defined in claim 1;
(B) R¹² is selected from one of the following options:
(i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
(ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
R¹² is —(CHRᵥ)_{d}—Z¹²
wherein R¹¹ is hydrogen;
wherein d is 0 or 1; and
Z¹² is —OR²⁰, —NR²¹R²² or —C(O)NR²¹R²²;
wherein R²⁰ is (1-4C)alkyl;
R²¹ and R²² are each independently selected from hydrogen, (1-6C)alkyl, (3-7C)cycloalkyl, phenyl, a carbon-linked 4 to 8-membered heterocyclyl, or a 5 or 6 membered heteroaryl;
or R²¹ and R²² are linked, such that, together with the nitrogen atom to which they are attached, they form a 4-8 membered heterocyclic ring;
wherein each of R²⁰, R²¹ or R²², or any ring formed when R²¹ and R²² are linked, is optionally substituted with one or more R^{a};
wherein R^{a} is as defined in claim 1;
(C) R¹² is selected from one of the following options:
(i) when R¹¹ is a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy, or
(ii) when R¹¹ is not a group —(CHR_{z})ₐ—Z¹¹ then R¹² is selected from halo, (1-2C)alkyl, (1-2C)alkoxy, wherein any (1-2C)alkyl or (1-2C)alkoxy is optionally substituted by one or more halo or (1-2C)alkoxy;
(D) R¹² is selected from fluoro, chloro, (1-2C)alkyl or (1-2C)alkoxy; or
(E) R¹² is fluoro, chloro or (1-2C)alkoxy.

18. A compound according to any one of claims 1 to 17, or pharmaceutically acceptable salt, hydrate or solvate thereof, wherein:
(A) R¹³ is selected from cyano, halo, (1-2C)alkyl, (1-2C)alkoxy, wherein any (1-2C)alkyl moiety is optionally substituted by one or more halo or (1-2C)alkoxy, or
R¹³ is —(CHRₒ)ₕ—Z¹³,
wherein R₀ is hydrogen or methyl;
wherein h is 0 or 1; and
Z¹³ is —OR²⁵, R^{25a}, —NR²⁶R²⁷, —C(O)NR²⁶R²⁷ or —NR²⁸C(O)R²⁹;
wherein R²⁵ is (1-4C)alkyl, (3-7C)cycloalkyl, phenyl, a carbon-linked 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ is hydrogen or methyl and i is 0 or 1;
R^{25a} is (1-4C)alkyl, (3-7C)cycloalkyl, phenyl, 4 to 8-membered heterocyclyl, a 5 or 6 membered heteroaryl, —(CHRₙ)ᵢ-(3-7C)cycloalkyl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ and i are as defined above;
R²⁶ and R²⁷ are each independently selected from hydrogen, (1-6C)alkyl, (2-6C)alkanoyl, (3-7C)cycloalkyl, phenyl, a carbon-linked 4 to 8-membered heterocyclyl, a 5 or 6 membered heteroaryl, —(CHRₘ)ⱼ-(3-7C)cycloalkyl, —(CHRₘ)ⱼ-phenyl, —(CHRₘ)ⱼ-[4 to 8-membered heterocyclyl] or —(CHRₘ)ⱼ-[5 or 6 membered heteroaryl], wherein Rₘ is hydrogen or methyl and j is 0 or 1;
or R²⁶ and R²⁷ are linked, such that, together with the nitrogen atom to which they are attached, they form a 4-8 membered heterocyclic ring;
R²⁸ is hydrogen or methyl;
R²⁹ is (1-6C)alkyl, (3-7C)cycloalkyl, phenyl, a carbon-linked 4 to 8-membered heterocyclyl, a 5 or 6 membered heteroaryl, —(CHRₗ)ₖ-(3-7C)cycloalkyl, —(CHRₗ)ₖ-phenyl, —(CHRₗ)ₖ-[4 to 8-membered heterocyclyl] or —(CHRₗ)ₖ-[5 or 6 membered heteroaryl], wherein Rₗ is hydrogen or methyl and k is 0 or 1;
wherein each of R²⁵, R^{25a}, R²⁶, R²⁷ or R²⁹ or any ring formed when R²⁶ and R²⁷ are linked, is optionally substituted with one or more R^{a};
wherein R^{a} is as defined in claim 1; or
(B) R¹³ is selected from halo, (1-2C)alkyl, (1-2C)alkoxy, wherein any (1-2C)alkyl moiety is optionally substituted by one or more halo or (1-2C)alkoxy, or
R¹³ is —(CHRₒ)ₕ—Z¹³
wherein Rₒ is hydrogen or methyl;
wherein h is 0 or 1; and
Z¹³ is —OR²⁵, R^{25a}, —NR²⁵R²⁷, or —C(O)NR²⁶R²⁷;
wherein R²⁵ is (1-4C)alkyl, a carbon-linked 4 to 8-membered heterocyclyl, or —(CHRₙ)ᵢ-[4 to 8-membered heterocyclyl], wherein Rₙ is hydrogen or methyl and i is 0 or 1;
R^{25a} is (1-4C)alkyl, phenyl, 4 to 8-membered heterocyclyl, a 5 or 6 membered heteroaryl, —(CHRₙ)ᵢ-phenyl, —(CHRₙ)ᵢ-[4 to 8-membered heterocyclyl] or —(CHRₙ)ᵢ-[5 or 6 membered heteroaryl], wherein Rₙ and i are as defined above;
R²⁶ and R²⁷ are each independently selected from hydrogen, (1-6C)alkyl, a carbon-linked 4 to 8-membered heterocyclyl, or —(CHRₘ)ⱼ-[4 to 8-membered heterocyclyl], wherein Rₘ is hydrogen or methyl and j is 0 or 1;
or R²⁶ and R²⁷ are linked, such that, together with the nitrogen atom to which they are attached, they form a 4-8 membered heterocyclic ring;
wherein each of R²⁵, R^{25a}, R²⁶, R²⁷ or any ring formed when R²⁶ and R²⁷ are linked, is optionally substituted with one or more R^{a};
wherein R^{a} is as defined in claim 1;
(C) R¹³ is —(CHRₒ)ₕ—Z¹³,
wherein Rₒ is hydrogen or methyl;
wherein h is 0 or 1; and
Z¹³ is —OR²⁵, R^{25a}, or —NR²⁶R²⁷;
wherein R²⁵ is (1-4C)alkyl;
R^{25a} is 4 to 8-membered heterocyclyl, a 5 or 6 membered heteroaryl, —(CHRₙ)ᵢ-[4 to 8-membered heterocyclyl] or —(CHRₙ)ᵢ-[5 or 6 membered heteroaryl], wherein Rₙ is hydrogen and i is 1 or 2;
R²⁶ and R²⁷ are each independently selected from hydrogen or (1-6C)alkyl;
or R²⁶ and R²⁷ are linked, such that, together with the nitrogen atom to which they are attached, they form a 4-8 membered heterocyclic ring;
wherein each of R²⁵, R^{25a}, R²⁶, R²⁷ or any ring formed when R²⁶ and R²⁷ are linked, is optionally substituted with one or more R^{a};
wherein R^{a} is as defined in claim 1;
(D) R¹³ is —(CHR₀)ₕ—Z¹³,
wherein Rₒ is hydrogen or methyl;
wherein h is 0 or 1; and
Z¹³ is —OR²⁵ or —NR²⁶R²⁷;
wherein R²⁵ is (1-4C)alkyl;
R²⁶ and R²⁷ are each independently selected from hydrogen or (1-6C)alkyl;
or R²⁶ and R²⁷ are linked, such that, together with the nitrogen atom to which they are attached, they form a 4-8 membered heterocyclic ring;
wherein each of R²⁵, R²⁶, R²⁷ or any ring formed when R²⁶ and R²⁷ are linked, is optionally substituted with one or more R^{a};
wherein R^{a} is as defined in claim 1;
(E) R¹³ is —(CHR₀)ₕ—Z¹³,
wherein Rₒ is hydrogen;
wherein h is 0 or 1; and
Z¹³ is —OR²⁵ or —NR²⁶R²⁷;
wherein R²⁵ is (1-2C)alkyl;
R²⁶ and R²⁷ are each independently selected from hydrogen or (1-2C)alkyl;
wherein each of R²⁵, R²⁶, R²⁷ or any ring formed when R²⁶ and R²⁷ are linked, is optionally substituted with one or more R^{a};
wherein R^{a} is as defined in claim 1.

19. A compound according to any one of claims 1 to 18, or pharmaceutically acceptable salt, hydrate or solvate thereof, wherein:
(i) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy, or
R¹⁴ is —(CHRₖ)ₘ—Z¹⁴,
wherein Rₖ is hydrogen;
wherein m is 0 or 1; and
Z¹⁴ is —OR³⁰, —NR³¹R³², —C(O)NR³¹R³² or —NR³³C(O)R³⁴;
wherein R³⁰ is (1-4C)alkyl, (3-7C)cycloalkyl, a carbon-linked 4 to 8-membered heterocyclyl, —(CHRⱼ)ₒ-(3-7C)cycloalkyl, or —(CHRⱼ)ₒ-[4 to 8-membered heterocyclyl], wherein Rⱼ is hydrogen or methyl and o is 0 or 1;
R³¹ and R³² are each independently selected from hydrogen, (1-6C)alkyl, (2-6C)alkanoyl, (3-7C)cycloalkyl, a carbon-linked 4 to 8-membered heterocyclyl, —(CHRᵢ)ₚ-(3-7C)cycloalkyl, or —(CHRᵢ)ₚ-[4 to 8-membered heterocyclyl], wherein Rᵢ is hydrogen or methyl and p is 0 or 1
or R³¹ and R³² are linked, such that, together with the nitrogen atom to which they are attached, they form a 4-8 membered heterocyclic ring;
R³³ is hydrogen or methyl;
R³⁴ is (1-6C)alkyl, (3-7C)cycloalkyl, a carbon-linked 4 to 8-membered heterocyclyl, —(CHRₕ)_{q}-(3-7C)cycloalkyl, or —(CHRₕ)_{q}-[4 to 8-membered heterocyclyl], wherein Rₕ is hydrogen or methyl and q is 0 or 1;
wherein R³⁰, R³¹, R³² or R³⁴, or any ring formed when R³¹ and R³² are linked, is optionally substituted with one or more R^{a};
wherein R^{a} is as defined in claim 1; or
(ii) R¹⁴ is selected from cyano, halo, (1-2C)alkyl, (1-2C)alkoxy, wherein any (1-2C)alkyl or (1-2C)alkoxy is optionally substituted by one or more halo or (1-2C)alkoxy.

20. A compound according to any one of claims 1 to 19, or pharmaceutically acceptable salt, hydrate or solvate thereof, wherein:
(i) each R^{a} is independently selected from the group consisting of oxo, halogen, cyano, hydroxy, (1-4C)alkyl, or a group 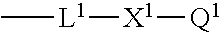
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
Q¹ is selected from the group consisting of hydrogen, (1-4C)alkyl, or (3-6C)cycloalkyl;
wherein when Q¹ is (1-4C)alkyl, or (3-6C)cycloalkyl, Q¹ is optionally substituted with one or more Q^{a}, where each Q^{a} is independently selected from the group consisting of hydroxy, cyano, halogen, (1-3C)alkyl, (1-3C)alkoxy, (1-3C)haloalkyl, or (1-3C)haloalkoxy;
(ii) each R^{a} is independently selected from the group consisting of oxo, halogen, cyano, hydroxy, (1-2C)alkyl, or a group 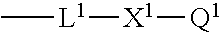
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, —NR¹⁴—, —SO₂N(R¹⁴)—, or —N(R¹⁴)SO₂—, where R¹⁴ is hydrogen or (1-2C)alkyl;
Q¹ is selected from the group consisting of hydrogen, (1-2C)alkyl, or (3-6C)cycloalkyl;
wherein when Q¹ is (1-2C)alkyl, or (3-6C)cycloalkyl, Q¹ is optionally substituted with one or more Q^{a}, where each Q^{a} is independently selected from the group consisting of hydroxy, halogen, (1-2C)alkyl, (1-2C)alkoxy, (1-2C)haloalkyl, or (1-2C)haloalkoxy;
(iii) each R^{a} is independently selected from the group consisting of oxo, halogen, cyano, hydroxy, (1-2C)alkyl, or a group 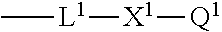
wherein:
L¹ is absent or (1-2C)alkylene;
X¹ is absent or is selected from the group consisting of —O—, —C(O)—, —C(O)—O—, —O—C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
Q¹ is selected from the group consisting of hydrogen or (1-2C)alkyl; or
wherein when Q¹ is (1-2C)alkyl, Q¹ is optionally substituted with one or more Q^{a}, where each Q^{a} is independently selected from the group consisting of hydroxy, halogen, or (1-2C)alkoxy;
(iv) each R^{a} is independently selected from the group consisting of oxo, halogen, cyano, hydroxy, (1-2C)alkyl, or a group 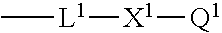
wherein:
L¹ is absent;
X¹ is absent or is selected from the group consisting of —O—, —C(O)—, —S(O)₀₋₂—, —C(O)—N(R¹⁴)—, —N(R¹⁴)—C(O)—, or —NR¹⁴—, where R¹⁴ is hydrogen or (1-2C)alkyl;
Q¹ is selected from the group consisting of hydrogen or (1-2C)alkyl;
wherein when Q¹ is (1-2C)alkyl, Q¹ is optionally substituted with one or more Q^{a}, where each Q^{a} is methoxy;
(v) each R^{a} is independently selected from the group consisting of oxo, halogen, cyano, hydroxy, (1-2C)alkyl, or a group 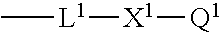
wherein:
L¹ is absent;
X¹ is absent or is selected from the group consisting of —O—, —C(O)— or —NR¹⁴— where R¹⁴ is hydrogen or (1-2C)alkyl;
Q¹ is selected from the group consisting of hydrogen or (1-2C)alkyl;
wherein when Q¹ is (1-2C)alkyl, Q¹ is optionally substituted with one or more Q^{a}, where each Q^{a} is methoxy; or
(vi) each R^{a} is independently selected from the group consisting of oxo, or a group 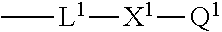
wherein:
L¹ is absent;
X¹ is absent or is selected from the group consisting of —O—, —C(O)— or —NR¹⁴— where R¹⁴ is hydrogen or (1-2C)alkyl;
Q¹ is selected from the group consisting of hydrogen or (1-2C)alkyl;
wherein when Q¹ is (1-2C)alkyl, Q¹ is optionally substituted with one or more Q^{a}, where each Q^{a} is methoxy.

21. The compound, or pharmaceutically acceptable salts thereof, according to any preceding claim, wherein the compound is selected from any one of the following:
(S)-(2-(Benzyloxy)-6-hydroxy-4-methoxyphenyl)(4-((tetrahydrofuran-3-yl)amino)isoindolin-2-yl)methanone
(R)-(2-(Benzyloxy)-6-hydroxy-4-methoxyphenyl)(4-((tetrahydrofuran-3-yl)amino)isoindolin-2-yl)methanone
(R)-(2-(Benzyloxy)-4-(difluoromethyl)-6-hydroxyphenyl)(4-((tetrahydrofuran-3-yl)amino)isoindolin-2-yl)methanone
(S)-(2-(Benzyloxy)-4-(difluoromethyl)-6-hydroxyphenyl)(4-((tetrahydrofuran-3-yl)amino)isoindolin-2-yl)methanone
(S)-(4-(Difluoromethyl)-2-hydroxy-6-((3-methoxybenzyl)oxy)phenyl)(4-((tetrahydrofuran-3-yl)amino)isoindolin-2-yl)methanone
(S)-(2-(Benzyloxy)-4-(difluoromethyl)-6-hydroxyphenyl)(6-((dimethylamino)methyl)-4-((tetrahydrofuran-3-yl)amino)isoindolin-2-yl)methanone
1-(3-((2-(2-(Benzyloxy)-4-(difluoromethyl)-6-hydroxybenzoyl)isoindolin-4-yl)amino)azetidin-1-yl)ethan-1-one
(2-(Benzyloxy)-4-(difluoromethyl)-6-hydroxyphenyl)(4-(oxetan-3-ylamino)isoindolin-2-yl)methanone
(4-(Difluoro methyl)-2-hydroxy-6-(pyridin-2-ylmethoxy) phenyl) (4-(oxetan-3-ylamino) isoindolin-2-yl) methanone
(S)-(2-(Benzyloxy)-4-(difluoromethyl)-6-hydroxyphenyl) (8-((tetrahydrofuran-3-yl)amino)-3,4-dihydroisoquinolin-2(1H)-yl)methanone
(2-(Benzyloxy)-4-(difluoromethyl)-6-hydroxyphenyl)(8-(oxetan-3-ylamino)-3,4-dihydroisoquinolin-2(1H)-yl) methanone
(S)-(4-(Difluoromethyl)-2-hydroxy-6-((4-methoxybenzyl)oxy)phenyl)(4-((tetrahydrofuran-3-yl)amino)isoindolin-2-yl)methanone
(S)-(4-(Difluoro methyl)-2-hydroxy-6-(pyridin-2-ylmethoxy) phenyl) (4-((tetrahydrofuran-3-yl) amino) isoindolin-2-yl) methanone
(S)-(4-(Difluoromethyl)-2-hydroxy-6-(pyridin-2-ylmethoxy)phenyl)(8-((tetrahydrofuran-3-yl)amino)-3,4-dihydroisoquinolin-2(1H)-yl)methanone
(S)-(4-(Difluoromethyl)-2-hydroxy-6-((4-methoxybenzyl)oxy)phenyl)(8-((tetrahydrofuran-3-yl)amino)-3,4-dihydroisoquinolin-2(1H)-yl)methanone
(S)-3-((2-(2-(Benzyloxy)-4-(difluoromethyl)-6-hydroxybenzoyl)isoindolin-4-yl)amino)-1-methylpyrrolidin-2-one
(S)-3-((2-(2-(benzyloxy)-4-(difluoromethyl)-6-hydroxybenzoyl)-1,2,3,4-tetrahydroisoquinolin-8-yl)amino)-1-methylpyrrolidin-2-one
(R)-3-((2-(2-(Benzyloxy)-4-(difluoromethyl)-6-hydroxybenzoyl)isoindolin-4-yl)amino)-1-methylpyrrolidin-2-one
(R)-3-((2-(2-(Benzyloxy)-4-(difluoromethyl)-6-hydroxybenzoyl)-1,2,3,4-tetrahydroisoquinolin-8-yl)amino)-1-methylpyrrolidin-2-one
3-((2-(2-(Benzyloxy)-4-(difluoromethyl)-6-hydroxybenzoyl) isoindolin-4-yl)amino)dihydrofuran-2(3H)-one
3-((2-(2-(Benzyloxy)-4-(difluoromethyl)-6-hydroxybenzoyl)-1,2,3,4-tetrahydroisoquinolin-8-yl) amino) dihydrofuran-2(3H)-one
(S)-(2-(Benzyloxy)-4-(difluoromethyl)-6-hydroxyphenyl) (6-methoxy-4-((tetrahydrofuran-3-yl)amino)isoindolin-2-yl)methanone
(S)-(6-((1H-Imidazol-1-yl) methyl)-4-((tetrahydrofuran-3-yl) amino) isoindolin-2-yl) (2-(benzyloxy)-4-(difluoromethyl)-6-hydroxyphenyl)methanone
(S)-(2-(Benzyloxy)-4-(difluoromethyl)-6-hydroxyphenyl) (6-methoxy-8-((tetrahydrofuran-3-yl)amino)-3,4-dihydroisoquinolin-2(1H)-yl)methanone
(S)-(2-(Benzyloxy)-4-(difluoromethyl)-6-hydroxyphenyl) (6-((dimethylamino)methyl)-8-((tetrahydrofuran-3-yl)amino)-3,4-dihydroisoquinolin-2(1H)-yl)methanone
(S)-(6-((1H-Imidazol-1-yl)methyl)-8-((tetrahydrofuran-3-yl)amino)-3,4-dihydroisoquinolin-2(1H)-yl)(2-(benzyloxy)-4-(difluoromethyl)-6-hydroxyphenyl)methanone
(S)-(2-(Benzyloxy)-4-(difluoromethyl)-6-hydroxyphenyl) (7-methoxy-8-((tetrahydrofuran-3-yl)amino)-3,4-dihydroisoquinolin-2(1H)-yl)methanone
(S)-(2-(Benzyloxy)-4-(difluoromethyl)-6-hydroxyphenyl)(6-(1-methyl-1H-imidazol-5-yl)-4-((tetrahydrofuran-3-yl)amino)isoindolin-2-yl)methanone
(S)-(2-(Benzyloxy)-4-(difluoromethyl)-6-hydroxyphenyl)(6-(1-methyl-1H-imidazol-4-yl)-4-((tetrahydrofuran-3-yl)amino)isoindolin-2-yl)methanone
(S)-(2-(Benzyloxy)-4-(difluoromethyl)-6-hydroxyphenyl) (6-(1-methyl-1H-imidazol-5-yl)-8-((tetrahydrofuran-3-yl) amino)-3,4-dihydroisoquinolin-2(1H)-yl)methanone
(S)-(2-(Benzyloxy)-4-(difluoromethyl)-6-hydroxyphenyl) (6-(1-methyl-1H-imidazol-4-yl)-8-((tetrahydrofuran-3-yl) amino)-3,4-dihydroisoquinolin-2(1H)-yl)methanone
2-(Benzyloxy)-4-(difluoromethyl)-6-hydroxyphenyl)(4-((2-methoxyethyl)amino)isoindolin-2-yl)methanone
2-((2-(2-(Benzyloxy)-4-(difluoro methyl)-6-hydroxybenzoyl) isoindolin-4-yl) amino)-N, N-dimethylacetamide
(2-(Benzyloxy)-4-(difluoromethyl)-6-hydroxyphenyl) (5-((4-methylpiperazin-1-yl) methyl) isoindolin-2-yl) methanone
(2-(Benzyloxy)-4-(difluoromethyl)-6-hydroxyphenyl)(5-(2-(dimethylamino)ethoxy)isoindolin-2-yl)methanone
(2-(Benzyloxy)-4-(difluoromethyl)-6-hydroxyphenyl)(5-((1-methylpiperidin-4-yl)oxy)isoindolin-2-yl)methanone
((2-(benzyloxy)-4-(difluoromethyl)-6-hydroxyphenyl)(4-((1-methylazetidin-3-yl)amino)isoindolin-2-yl)methanone
(2-(Benzyloxy)-4-(difluoro methyl)-6-hydroxyphenyl) (4-(methylamino) isoindolin-2-yl) methanone
(2-(Benzyloxy)-4-(difluoromethyl)-6-hydroxyphenyl)(3,4-dihydroisoquinolin-2(1H)-yl)methanone
(2-(Benzyloxy)-4-(difluoromethyl)-6-hydroxyphenyl)(8-(methylamino)-3,4-dihydroisoquinolin-2(1H)-yl)methanone
(2-(Benzyloxy)-4-(difluoromethyl)-6-hydroxyphenyl)(8-((2-methoxyethyl)amino)-3,4-dihydroisoquinolin-2(1H)-yl)methanone
(2-(Benzyloxy)-4-(difluoromethyl)-6-hydroxyphenyl)(7-methoxy-3,4-dihydroisoquinolin-2(1H)-yl)methanone
(2-(Benzyloxy)-4-(difluoromethyl)-6-hydroxyphenyl)(6-methoxy-3,4-dihydroisoquinolin-2(1H)-yl)methanone
(2-(Benzyloxy)-4-(difluoromethyl)-6-hydroxyphenyl)(7-(2-(dimethylamino)ethoxy)-3,4-dihydroisoquinolin-2(1H)-yl)methanone
(2-(Benzyloxy)-4-(difluoromethyl)-6-hydroxyphenyl) (6-(2-(dimethylamino)ethoxy)-3,4-dihydroisoquinolin-2(1H)-yl)methanone
(2-(Benzyloxy)-4-(difluoromethyl)-6-hydroxyphenyl)(7-((dimethylamino)methyl)-3,4-dihydroisoquinolin-2(1H)-yl)methanone
(2-(Benzyloxy)-4-(difluoromethyl)-6-hydroxyphenyl)(6-((dimethylamino)methyl)-3,4-dihydroisoquinolin-2(1H)-yl)methanone
(2-(Benzyloxy)-4-(difluoromethyl)-6-hydroxyphenyl)(7-((1-methyl piperidin-4-yl)oxy)-3,4-dihydroisoquinolin-2(1H)-yl)methanone
(2-(Benzyloxy)-4-(difluoromethyl)-6-hydroxyphenyl)(6-((1-methyl piperidin-4-yl)oxy)-3,4-dihydroisoquinolin-2(1H)-yl)methanone
(2-(Benzyloxy)-4-(difluoromethyl)-6-hydroxyphenyl)(7-((4-methyl piperazin-1-yl)methyl)-3,4-dihydroisoquinolin-2(1H)-yl)methanone
(2-(Benzyloxy)-4-(difluoromethyl)-6-hydroxyphenyl)(6-((4-methyl piperazin-1-yl)methyl)-3,4-dihydroisoquinolin-2(1H)-yl)methanone
(7-((1H-Imidazol-1-yl)methyl)-3,4-dihydroisoquinolin-2(1H)-yl)(2-(benzyloxy)-4-(difluoromethyl)-6-hydroxyphenyl)methanone
(6-((1H-Imidazol-1-yl)methyl)-3,4-dihydroisoquinolin-2(1H)-yl)(2-(benzyloxy)-4-(difluoromethyl)-6-hydroxyphenyl)methanone
(5-((1H-Imidazol-1-yl)methyl)isoindolin-2-yl)(2-(benzyloxy)-4-(difluoromethyl)-6-hydroxyphenyl)methanone
(2-(Benzyloxy)-4-(difluoromethyl)-6-hydroxyphenyl)(5-((dimethylamino)methyl)isoindolin-2-yl)methanone
(2-(Benzyloxy)-4-(difluoromethyl)-6-hydroxyphenyl)(2-((dimethylamino)methyl)-5,7-dihydro-6H-pyrrolo[3,4-b]pyridin-6-yl)methanone
(2-(Benzyloxy)-4-(difluoromethyl)-6-hydroxyphenyl)(3-((dimethylamino)methyl)-5,7-dihydro-6H-pyrrolo[3,4-b]pyridin-6-yl)methanone
(2-(Benzyloxy)-4-(difluoromethyl)-6-hydroxyphenyl)(isoindolin-2-yl)methanone
(R)-(2-(Benzyloxy)-4-(difluoromethyl)-6-hydroxyphenyl)(5-((1-methylpyrrolidin-3-yl)oxy)isoindolin-2-yl)methanone
(S)-(2-(Benzyloxy)-4-(difluoromethyl)-6-hydroxyphenyl) (5-((1-methylpyrrolidin-3-yl)oxy)isoindolin-2-yl)methanone
2-(Benzyloxy)-4-(difluoromethyl)-6-hydroxyphenyl) (5-((1-methylazetidin-3-yl)oxy)isoindolin-2-yl) methanone
(4-(Difluoromethyl)-2-hydroxy-6-(pyridin-2-ylmethoxy)phenyl)(5-((1-methylpiperidin-4-yl)oxy)isoindolin-2-yl)methanone
(2-(Cyclopropylmethoxy)-4-(difluoromethyl)-6-hydroxyphenyl)(5-((1-methylpiperidin-4-yl)oxy)isoindolin-2-yl)methanone
(2-(Benzyloxy)-4-(difluoromethyl)-6-hydroxyphenyl)(5-(pyridin-4-yloxy)isoindolin-2-yl)methanone
(2-(Cyclopropylmethoxy)-4-(difluoromethyl)-6-hydroxyphenyl)(5-((1-methylpiperidin-4-yl)oxy)isoindolin-2-yl)methanone
(S)-(2-(Benzyloxy)-4-(difluoromethyl)-6-hydroxyphenyl) (6-((1-methyl piperidin-4-yl)oxy)-4-((tetrahydrofuran-3-yl)amino)isoindolin-2-yl)methanone
(R)-(2-(Benzyloxy)-4-(difluoromethyl)-6-hydroxyphenyl)(6-((1-methylpiperidin-4-yl)oxy)-4-((tetrahydrofuran-3-yl)amino)isoindolin-2-yl)methanone
(S)-(2-(Benzyloxy)-4-(difluoromethyl)-6-hydroxyphenyl) (6-((1-methylpyrrolidin-3-yl)oxy)-3,4-dihydroisoquinolin-2(1H) yl)methanone
(R)-(2-(Benzyloxy)-4-(difluoromethyl)-6-hydroxyphenyl)(6-((1-methylpyrrolidin-3-yl)oxy)-3,4-dihydroisoquinolin-2(1H) yl)methanone
(2-(Benzyloxy)-4-(difluoromethyl)-6-hydroxyphenyl)(6-((1-methylazetidin-3-yl)oxy)-3,4-dihydroisoquinolin-2(1H)-yl)methanone
(R)-(2-(Benzyloxy)-4-(difluoromethyl)-6-hydroxyphenyl)(7-((1-methylpyrrolidin-3-yl)oxy)-3,4-dihydroisoquinolin-2(1H)-yl)methanone
(S)-(2-(Benzyloxy)-4-(difluoromethyl)-6-hydroxyphenyl) (7-((1-methylpyrrolidin-3-yl)oxy)-3,4-dihydroisoquinolin-2(1H)-yl)methanone
(2-(Benzyloxy)-4-(difluoromethyl)-6-hydroxyphenyl)(7-((1-methylazetidin-3-yl)oxy)-3,4-dihydroisoquinolin-2(1H)-yl)methanone
(2-(Benzyloxy)-4-(difluoro methyl)-6-hydroxyphenyl) (5-(2-morpholinoethoxy) isoindolin-2-yl) methanone
(2-(Benzyloxy)-4-(difluoromethyl)-6-hydroxyphenyl) (5-(2-(4-methyl piperazin-1-yl) ethoxy) isoindolin-2-yl) methanone
(2-(Benzyloxy)-4-(difluoromethyl)-6-hydroxyphenyl)(8-(cyclopentylamino)-6-(2-(dimethylamino)ethoxy)-3,4-dihydroisoquinolin-2(1H)-yl)methanone
(2-(Benzyloxy)-4-(difluoromethyl)-6-hydroxyphenyl)(6-(2-(dimethylamino)ethoxy)-8-(methylamino)-3,4-dihydroisoquinolin-2(1H)-yl)methanone
(2-(Benzyloxy)-4-(difluoromethyl)-6-hydroxyphenyl)(8-(difluoromethyl)-6-(2-(dimethylamino)ethoxy)-3,4-dihydroisoquinolin-2(1H)-yl)methanone
(R)-(2-(Benzyloxy)-4-(difluoromethyl)-6-hydroxyphenyl)(6-(2-(dimethylamino)ethoxy)-4-((tetrahydrofuran-3-yl)amino)isoindolin-2-yl)methanone
((S)-(2-(Benzyloxy)-4-(difluoromethyl)-6-hydroxyphenyl)(6-(2-(dimethylamino)ethoxy)-4-((tetrahydrofuran-3-yl)amino)isoindolin-2-yl)methanone
(2-(Benzyloxy)-4-(difluoromethyl)-6-hydroxyphenyl)(5-(2-methoxyethoxy)isoindolin-2-yl)methanone
(2-(Benzyloxy)-4-(difluoromethyl)-6-hydroxyphenyl)(5-((tetrahydro-2H-pyran-4-yl)oxy)isoindolin-2-yl)methanone
(2-(Benzyloxy)-4-(difluoromethyl)-6-hydroxyphenyl)(4-((tetrahydrofuran-3-yl)oxy)isoindolin-2-yl)methanone
2-(Benzyloxy)-4-(difluoromethyl)-6-hydroxyphenyl) (8-((tetrahydrofuran-3-yl)oxy)-3,4-dihydroisoquinolin-2(1H)-yl)methanone
(S)-(2-(Benzyloxy)-4-fluoro-6-hydroxyphenyl)(4-((tetrahydrofuran-3-yl)amino)isoindolin-2-yl)methanone
(R)-(2-(Benzyloxy)-4-fluoro-6-hydroxyphenyl) (4-((tetrahydrofuran-3-yl)amino) isoindolin-2-yl)methanone
(R)-(2-(Benzyloxy)-4-chloro-6-hydroxyphenyl) (4-((tetrahydrofuran-3-yl)amino) isoindolin-2-yl)methanone
(S)-(2-(Benzyloxy)-4-chloro-6-hydroxyphenyl)(4-((tetrahydrofuran-3-yl)amino)isoindolin-2-yl)methanone
(R)-(2-(Benzyloxy)-4-chloro-6-hydroxyphenyl) (6-(2-(dimethylamino)ethoxy)-4-((tetrahydrofuran-3-yl)amino)isoindolin-2-yl)methanone
(S)-(2-(Benzyloxy)-4-chloro-6-hydroxyphenyl)(6-(2-(dimethylamino)ethoxy)-4-((tetrahydrofuran-3-yl)amino)isoindolin-2-yl)methanone
(R)-(2-(Benzyloxy)-4-chloro-6-hydroxyphenyl) (6-methoxy-4-((tetrahydrofuran-3-yl)amino)isoindolin-2-yl)methanone
(S)-(2-(benzyloxy)-4-chloro-6-hydroxyphenyl)(6-methoxy-4-((tetrahydrofuran-3-yl)amino)isoindolin-2-yl)methanone
3-(Benzyloxy)-5-hydroxy-4-(4-((1-methyl-5-oxopyrrolidin-3-yl)amino)isoindoline-2-carbonyl)benzonitrile
3-(Benzyloxy)-5-hydroxy-4-(4-(pyrimidin-5-ylamino)isoindoline-2-carbonyl)benzonitrile
(R)-4-(4-((1-Acetylpyrrolidin-3-yl)amino)isoindoline-2-carbonyl)-3-(benzyloxy)-5-hydroxybenzonitrile
(S)-4-(4-((1-Acetylpyrrolidin-3-yl)amino)isoindoline-2-carbonyl)-3-(benzyloxy)-5-hydroxybenzonitrile
(R)-3-(Benzyloxy)-5-hydroxy-4-(4-((1-methyl-5-oxopyrrolidin-3-yl)amino)isoindoline-2-carbonyl)benzonitrile or (S)-3-(benzyloxy)-5-hydroxy-4-(4-((1-methyl-5-oxopyrrolidin-3-yl)amino)isoindoline-2-carbonyl)benzonitrile
3-(Benzyloxy)-5-hydroxy-4-(4-((oxazol-4-ylmethyl)amino)isoindoline-2-carbonyl)benzonitrile
3-(Benzyloxy)-5-hydroxy-4-(4-(((tetrahydrofuran-3-yl)methyl)amino) isoindoline-2-carbonyl)benzonitrile
3-(Benzyloxy)-5-hydroxy-4-(4-methoxyisoindoline-2-carbonyl) benzonitrile
(R)-3-(Benzyloxy)-5-hydroxy-4-(8-((tetrahydrofuran-3-yl)amino)-1,2,3,4-tetrahydroisoquinoline-2-carbonyl)benzonitrile
(R)-3-(benzyloxy)-5-hydroxy-4-(6-methoxy-4-((tetrahydrofuran-3-yl)amino)isoindoline-2-carbonyl)benzonitrile
(S)-3-(Benzyloxy)-5-hydroxy-4-(6-methoxy-4-((tetrahydrofuran-3-yl)amino) isoindoline-2-carbonyl)benzonitrile
3-(Benzyloxy)-4-(5-((dimethylamino)methyl)isoindoline-2-carbonyl)-5-hydroxybenzonitrile
4-(4-((1-Acetylpiperidin-4-yl)amino)isoindoline-2-carbonyl)-3-(benzyloxy)-5-hydroxybenzonitrile
(R)-4-(4-((1-Acetylpiperidin-3-yl)amino)isoindoline-2-carbonyl)-3-(benzyloxy)-5-hydroxybenzonitrile
(S)-4-(4-((1-Acetylpiperidin-3-yl)amino) isoindoline-2-carbonyl)-3-(benzyloxy)-5-hydroxybenzonitrile
3-(Benzyloxy)-5-hydroxy-4-(4-((5-oxotetrahydrofuran-3-yl)amino) isoindoline-2-carbonyl)benzonitrile
(2-(Benzyloxy)-4-chloro-6-hydroxyphenyl) (4-((1-methylazetidin-3-yl)amino)isoindolin-2-yl) methanone
2-(Benzyloxy)-4-chloro-6-hydroxyphenyl) (2-((dimethylamino)methyl)-5,7-dihydro-6H-pyrrolo[3,4-b]pyridin-6-yl)methanone
(2-(Benzyloxy)-4-chloro-6-hydroxyphenyl) (6-(2-(dimethylamino)ethoxy)-3,4-dihydroisoquinolin-2(1H)-yl)methanone
1-(3-((2-(2-(Benzyloxy)-4-fluoro-6-hydroxybenzoyl) isoindolin-4-yl)amino)azetidin-1-yl)ethan-1-one
(2-(Benzyloxy)-4-fluoro-6-hydroxyphenyl)(6-(2-(dimethylamino)ethoxy)-3,4-dihydroisoquinolin-2(1H)-yl)methanone
(2-(Benzyloxy)-4-fluoro-6-hydroxyphenyl) (5-((1-methylpiperidin-4-yl) oxy) isoindolin-2-yl) methadone
3-(Benzyloxy)-5-hydroxy-4-(4-(oxetan-3-ylamino)isoindoline-2-carbonyl)benzonitrile
(S)-3-(Benzyloxy)-5-hydroxy-4-(4-((1-methyl-2-oxopyrrolidin-3-yl)amino)isoindoline-2-carbonyl)benzonitrile
(R)-3-(benzyloxy)-5-hydroxy-4-(4-((1-methyl-2-oxopyrrolidin-3-yl)amino) isoindoline-2-carbonyl)benzonitrile
3-(Benzyloxy)-4-(6-(2-(dimethyl amino) ethoxy)-1,2,3,4-tetrahydroisoquinoline-2-carbonyl)-5-hydroxybenzonitrile
(R)-3-(Benzyloxy)-5-hydroxy-4-(5-((1-methylpyrrolidin-3-yl)oxy)isoindoline-2-carbonyl)benzonitrile
(S)-3-(Benzyloxy)-5-hydroxy-4-(5-((1-methylpyrrolidin-3-yl)oxy)isoindoline-2-carbonyl)benzonitrile
3-(Benzyloxy)-5-hydroxy-4-(5-((1-methylazetidin-3-yl)oxy)isoindoline-2-carbonyl)benzonitrile
3-(Benzyloxy)-5-hydroxy-4-(5-((1-methyl piperidin-4-yl)oxy)isoindoline-2-carbonyl)benzonitrile
3-(Benzyloxy)-5-hydroxy-4-(5-((4-methyl piperazin-1-yl)methyl)isoindoline-2-carbonyl)benzonitrile
3-(Benzyloxy)-5-hydroxy-4-(5-(2-methoxyethoxy) isoindoline-2-carbonyl)benzonitrile
3-(Benzyloxy)-5-hydroxy-4-(5-((tetrahydro-2H-pyran-4-yl)oxy) isoindoline-2-carbonyl)benzonitrile
3-Hydroxy-4-(5-((1-methylazetidin-3-yl)oxy)isoindoline-2-carbonyl)-5-(pyridin-2-ylmethoxy)benzonitrile
3-Hydroxy-4-(4-(oxetan-3-ylamino) isoindoline-2-carbonyl)-5-(pyridin-2-ylmethoxy)benzonitrile
(S)-3-Hydroxy-5-(pyridin-2-ylmethoxy)-4-(4-((tetrahydrofuran-3-yl)amino) isoindoline-2-carbonyl)benzonitrile
(S)-3-(Benzyloxy)-5-hydroxy-4-(4-((tetrahydrofuran-3-yl)amino) isoindoline-2-carbonyl)benzonitrile
(S)-3-Hydroxy-5-methoxy-4-(4-((tetrahydrofuran-3-yl)amino)isoindoline-2-carbonyl)benzonitrile
(S)-3-(Cyclopropylmethoxy)-5-hydroxy-4-(4-((tetrahydrofuran-3-yl)amino)isoindoline-2-carbonyl)benzonitrile
(R)-3-(Benzyloxy)-5-hydroxy-4-(4-((tetrahydrofuran-3-yl)amino) isoindoline-2-carbonyl)benzonitrile
3-(Benzyloxy)-5-hydroxy-4-(isoindoline-2-carbonyl)benzonitrile
3-(Benzyloxy)-5-hydroxy-4-(5-methoxyisoindoline-2-carbonyl)benzonitrile
(R)-3-(Benzyloxy)-4-(6-(2-(dimethylamino)ethoxy)-8-((tetrahydrofuran-3-yl)amino)-1,2,3,4-tetrahydroisoquinoline-2-carbonyl)-5-hydroxybenzonitrile
(S)-3-(Benzyloxy)-4-(6-(2-(dimethylamino)ethoxy)-8-((tetrahydrofuran-3-yl)amino)-1,2,3,4-tetrahydroisoquinoline-2-carbonyl)-5-hydroxybenzonitrile
4-(4-((1-Acetylazetidin-3-yl)amino)isoindoline-2-carbonyl)-3-(benzyloxy)-5-hydroxybenzonitrile
(S)-3-(Benzyloxy)-5-hydroxy-4-(8-((tetrahydrofuran-3-yl)amino)-1,2,3,4-tetrahydroisoquinoline-2-carbonyl)benzonitrile
(R)-3-(Benzyloxy)-4-(6-(2-(dimethylamino)ethoxy)-4-((tetrahydrofuran-3-yl)amino) isoindoline-2-carbonyl)-5-hydroxybenzonitrile
(S)-3-(Benzyloxy)-4-(6-(2-(dimethylamino)ethoxy)-4-((tetrahydrofuran-3-yl)amino)isoindoline-2-carbonyl)-5-hydroxybenzonitrile
(R)-(2-(Benzyloxy)-4-chloro-6-hydroxyphenyl) (6-(2-(dimethylamino)ethoxy)-8-((tetrahydrofuran-3-yl)amino)-3,4-dihydroisoquinolin-2(1H)-yl)methanone
(S)-(2-(Benzyloxy)-4-chloro-6-hydroxyphenyl)(6-(2-(dimethylamino)ethoxy)-8-((tetrahydrofuran-3-yl)amino)-3,4-dihydroisoquinolin-2(1H)-yl)methanone
(S)-(2-(Benzyloxy)-6-hydroxy-4-(trifluoromethyl) phenyl) (4-((tetrahydrofuran-3-yl) amino) isoindolin-2-yl) methanone
(S)-(2-(Benzyloxy)-4-(difluoromethyl)-6-hydroxyphenyl)(6-(2-(dimethylamino)ethoxy)-8-((tetrahydrofuran-3-yl)amino)-3,4-dihydroisoquinolin-2(1H)-yl)methanone
(R)-(2-(Benzyloxy)-4-(difluoromethyl)-6-hydroxyphenyl)(6-(2-(dimethylamino)ethoxy)-8-((tetrahydrofuran-3-yl)amino)-3,4-dihydroisoquinolin-2(1H)-yl)methanone
(2-(Benzyloxy)-4-(difluoromethyl)-6-hydroxyphenyl)(5-(piperidin-4-yloxy)isoindolin-2-yl)methanone
3-(Benzyloxy)-4-(6-((dimethylamino)methyl)-2,3-dihydro-1H-pyrrolo[3,4-c]pyridine-2-carbonyl)-5-hydroxybenzonitrile
3-(Benzyloxy)-5-hydroxy-4-(6-(4-methylpiperazin-1-yl)-2,3-dihydro-1H-pyrrolo[3,4-c]pyridine-2-carbonyl)benzonitrile
(2-(Benzyloxy)-4-(difluoromethyl)-6-hydroxyphenyl)(6-((tetrahydrofuran-3-yl)amino)indolin-1-yl)methanone
(S or R)-(2-(Benzyloxy)-4-(difluoromethyl)-6-hydroxyphenyl)(6-((tetrahydrofuran-3-yl)amino)indolin-1-yl)methanone
3-(Benzyloxy)-5-hydroxy-4-(6-((tetrahydrofuran-3-yl)amino)indoline-1-carbonyl)benzonitrile
3-(Benzyloxy)-5-hydroxy-4-(5-((tetrahydrofuran-3-yl)amino)indoline-1-carbonyl)benzonitrile
(2-(Benzyloxy)-4-(difluoromethyl)-6-hydroxyphenyl)(5-((tetrahydrofuran-3-yl)amino)indolin-1-yl)methanone
(S or R)-(2-(Benzyloxy)-4-(difluoromethyl)-6-hydroxyphenyl)(5-((tetrahydrofuran-3-yl)amino)indolin-1-yl)methanone
3-(Benzyloxy)-5-hydroxy-4-(7-((1-methyl piperidin-4-yl)oxy)-2,3,4,5-tetrahydro-1H-benzo[d]azepine-3-carbonyl)benzonitrile hydroformate
(2-(Benzyloxy)-4-(difluoromethyl)-6-hydroxyphenyl)(7-((1-methyl piperidin-4-yl)oxy)-1,2,4,5-tetrahydro-3H-benzo[d]azepin-3-yl)methanone
3-(Benzyloxy)-5-hydroxy-4-(6-morpholino-2,3-dihydro-1H-pyrrolo[3,4-c]pyridine-2-carbonyl)benzonitrile
(2-(Benzyloxy)-4-(difluoromethyl)-6-hydroxyphenyl)(6-morpholino-1,3-dihydro-2H-pyrrolo[3,4-c]pyridin-2-yl)methanone
4-(6-Amino-2,3-dihydro-1H-pyrrolo[3,4-c]pyridine-2-carbonyl)-3-(benzyloxy)-5-hydroxybenzonitrile hydroformate
(6-Amino-1,3-dihydro-2H-pyrrolo[3,4-c]pyridin-2-yl)(2-(benzyloxy)-4-(difluoromethyl)-6-hydroxyphenyl)methanone hydroformate
3-(Benzyloxy)-4-(4-(2-(dimethylamino)ethoxy)indoline-1-carbonyl)-5-hydroxybenzonitrile
(2-(Benzyloxy)-4-(difluoromethyl)-6-hydroxyphenyl)(4-(2-(dimethylamino)ethoxy)indolin-1-yl)methanone
3-(Benzyloxy)-4-(2,3-dihydro-1H-pyrrolo[2,3-b]pyridine-1-carbonyl)-5-hydroxybenzonitrile
(2-(Benzyloxy)-4-(difluoromethyl)-6-hydroxyphenyl)(2,3-dihydro-1H-pyrrolo[2,3-b]pyridin-1-yl)methanone
3-(Benzyloxy)-4-(2-(2-(dimethylamino)ethoxy)-4-((tetrahydrofuran-3-yl)amino)-5,6,7,8-tetrahydropyrido[4,3-d]pyrimidine-6-carbonyl)-5-hydroxybenzonitrile
(2-(Benzyloxy)-4-(difluoromethyl)-6-hydroxyphenyl)(2-(2-(dimethylamino)ethoxy)-4-((tetrahydrofuran-3-yl)amino)-7,8-dihydropyrido[4,3-d]pyrimidin-6(5H)-yl)methanone
3-(Benzyloxy)-5-hydroxy-4-(6-((tetrahydrofuran-3-yl)amino)-1,2,3,4-tetrahydroquinoline-1-carbonyl)benzonitrile
(2-(Benzyloxy)-4-(difluoromethyl)-6-hydroxyphenyl)(6-((tetrahydrofuran-3-yl)amino)-3,4-dihydroquinolin-1(2H)-yl)methanone
(2-(Benzyloxy)-4-(difluoromethyl)-6-hydroxyphenyl)(4-((tetrahydrofuran-3-yl)amino)-7,8-dihydropyrido[4,3-d]pyrimidin-6(5H)-yl)methanone
(2-(Benzyloxy)-4-(difluoromethyl)-6-hydroxyphenyl)(2,3-dihydro-1H-pyrrolo[3,2-c]pyridin-1-yl)methanone
(2-(Benzyloxy)-4-(difluoromethyl)-6-hydroxyphenyl)(2,3-dihydro-1H-pyrrolo[3,2-b]pyridin-1-yl)methanone
(2-(Benzyloxy)-4-(difluoromethyl)-6-hydroxyphenyl)(2,3-dihydro-1H-pyrrolo[2,3-c]pyridin-1-yl)methanone
(2-(Benzyloxy)-4-(difluoromethyl)-6-hydroxyphenyl)(4-((tetrahydrofuran-3-yl)amino)indolin-1-yl)methanone
(S)-(4-(Difluoromethyl)-2-hydroxy-6-((1-methyl-1H-indazol-6-yl)methoxy)phenyl)(8-((tetrahydrofuran-3-yl)amino)-3,4-dihydroisoquinolin-2(1H)-yl)methanone
(S)-(2-((2-Oxabicyclo[2.1.1]hexan-1-yl)methoxy)-4-(difluoromethyl)-6-hydroxyphenyl) (8-((tetrahydrofuran-3-yl)amino)-3,4-dihydroisoquinolin-2(1H)-yl)methanone
(S)-(4-(Difluoromethyl)-2-hydroxy-6-(isoquinolin-6-ylmethoxy)phenyl)(8-((tetrahydrofuran-3-yl)amino)-3,4-dihydroisoquinolin-2(1H)-yl)methanone
(S)-(4-(Difluoromethyl)-2-hydroxy-6-(isoquinolin-7-ylmethoxy)phenyl)(8-((tetrahydrofuran-3-yl)amino)-3,4-dihydroisoquinolin-2(1H)-yl)methanone
(S)-(2-((1H-Benzo[d]imidazol-2-yl)methoxy)-4-(difluoromethyl)-6-hydroxyphenyl)(8-((tetrahydrofuran-3-yl)amino)-3,4-dihydroisoquinolin-2(1H)-yl)methanone.

22. A pharmaceutical composition comprising a compound as defined in any one of the preceding claims, or a pharmaceutically acceptable salt thereof, in admixture with a pharmaceutically acceptable diluent or carrier.

23. A compound, or a pharmaceutically acceptable salt thereof, as defined in any one of claims 1-21, or a pharmaceutical composition as defined in claim 22:
(i) for use in therapy;
(ii) the treatment of cancer;
(iii) the treatment of cancer, wherein the compound or pharmaceutical composition is administered in combination with another anticancer agent (e.g. a chemotherapeutic agent, an immune checkpoint inhibitor, an immune stimulator or DNA damage repair modulator);
(iv) the treatment of a triplet repeat disorder.

24. A method of treating cancer or a triplet repeat disorder in a patient in need of such treatment, the method involving administering a therapeutically effective amount of a compound, or a pharmaceutically acceptable salt thereof, as defined in any one of claims 1-21, or a pharmaceutical composition as defined in claim 22.

25. A method of treating cancer according to claim 24, wherein the method comprises administering a therapeutically effective amount of a compound, or a pharmaceutically acceptable salt thereof, as defined in any one of claims 1-21, or a pharmaceutical composition as defined in claim 22, in combination with another anticancer agent (e.g. a chemotherapeutic agent, an immune checkpoint inhibitor, an immune stimulator or DNA damage repair modulator).
